(19) United States
(12) Patent Application Publication
(10) Pub. No.: US 2021/0300936 A1
(43) Pub. Date: Sep. 30, 2021

(54) PYRAZOLOTRIAZOLOPYRIMIDINE DERIVATIVES AS A2A RECEPTOR ANTAGONIST

(71) Applicant: BEIGENE, LTD., Camana Bay, Grand Cayman (KY)

(72) Inventors: Guoliang ZHANG, Beijing (CN); Hanzi SUN, Beijing (CN); Changyou ZHOU, Princeton, NJ (US)

(21) Appl. No.: 17/261,359

(22) PCT Filed: Jul. 22, 2019
(86) PCT No.: PCT/CN2019/097083

§ 371 Date: Jan. 19, 2021

(30) Foreign Application Priority Data
Jul. 23, 2018 (CN) PCT/CN2018/096633

(51) Int. Cl. C07D 487/14

(52) U.S. Cl. CPC C07D 487/14

(57) ABSTRACT

Disclosed herein is a pyrazolotriazolopyrimidine derivative or a stereoisomer thereof, or a pharmaceutically acceptable salt thereof useful as A2A receptor antagonist, and a pharmaceutical composition comprising the same. Also disclosed herein is a method of treating cancer using the pyrazolotriazolopyrimidine derivative or a stereoisomer thereof, or a pharmaceutically acceptable salt thereof as A2A receptor antagonist.

### CROSS-REFERENCE TO RELATED APPLICATIONS

This application claims the benefit of International Patent Application No. PCT/CN2018/096633 filed on Jul. 23, 2018, the disclosures of which are hereby incorporated by reference in its entirety for all purposes.

### FIELD OF THE INVENTION

Disclosed herein is a pyrazolotriazolopyrimidine derivative or a stereoisomer thereof, or a pharmaceutically acceptable salt thereof useful as A2A receptor antagonist, and a pharmaceutical composition comprising the same. Also disclosed herein is a method of treating cancer using the pyrazolotriazolopyrimidine derivative or a stereoisomer thereof, or a pharmaceutically acceptable salt thereof as A2A receptor antagonist.

### BACKGROUND OF THE INVENTION

Extracellular adenosine is a key endogenous modulator of a number of physiological activities. It exerts its regulatory function by interacting with four adenosine receptors, A1, A2A, A2B and A3. All the four receptors belong to G-protein-coupled receptor superfamily but have different ligand affinities, tissue distributions and effector responses. By coupling with different G proteins, they can either stimulate (A2A and A2B) or inhibit (A1 and A3) downstream adenylyl cyclase activity, and also involved in regulating other pathways, such as phospholipase C (PLC), Ca²⁺ and mitogen-activated protein kinases (MAPKs).

The immune system is not only responsible for defending its host against microbial invasion, but also can remove the changed host component from an organism, where an anti-tumor immune mechanism exists. When the immune surveillance function is weakened due to the immune system per se or tumor cells, favorable conditions are provided for the development and progression of tumors. Adenosine-A2A receptor signaling emerges as a novel metabolic immune checkpoint pathway that participates in creation of an immune-tolerant tumor microenvironment. It was demonstrated that the hypoxia in tumor tissue would induce the accumulation of higher concentrations of adenosine (˜10 μM versus ˜20 nM at physiologically level). Hypoxia-mediated adenosine production was caused by upregulation of CD39 and CD73 ectonucleotidase in both non-hematopoietic and hematopoietic cellular subsets, which sequentially catalyzed the conversion of extracellular ATP to adenosine.

Adenosine signaling through A2A (high affinity) and A2B (low affinity) receptors-major adenosine receptors in immune cell subsets-plays an important role in protecting cancerous tissues from the attack of the immune system. Activated A2A receptors on T effector cells increase intracellular cAMP, which in turn suppresses TCR-triggered signaling and anti-tumor effector function, including reduced T cell expansion, IFN-γ releasing, and increased expression of immunosuppressive PD-1, LAG3, IL-10 and TGF-β. Increased cAMP in T cells also promotes cAMP-response element (CRE)-mediated transcription, such as FoxP3, which drives regulatory T cell phenotype. Besides, adenosine also inhibits anti-tumor immune response by disabling the cytotoxic effector function of natural killer (NK) cells, regulating immunosuppressive M2 macrophage polarization and myeloid-derived suppressor cells (MDSC) expansion. Thus, immune cell expressing A2A receptors were investigated as a potential target to disrupt adenosine-mediated immunosuppression in the tumor microenvironment. It was demonstrated that genetic deletion or pharmacological antagonism of A2AR could enhance endogenous antitumor immunity and effectively inhibited tumor growth or metastasis in established immunogenic mouse tumors.

WO0192264 disclosed the 5-amino-pyrazolo-[4,3-e]-1,2,4-triazolo[1,5-c]pyrimidine adenosine A2a receptor antagonists for the treatment of central nervous system diseases, in particular Parkinson's disease, which was proved to have a high Blood-brain Barrier Permeability.

However, there is a need of small molecule antagonists of the A2A receptor as immune modulators for anticancer therapy (Robert D. Leone, Ying-Chun Lo, Jonathan D. Powell, Mini Review, A2aR antagonists: Next generation checkpoint blockade for cancer immunotherapy, Computational and Structural Biotechnology Journal 13 (2015) 265-272).

### SUMMARY OF THE INVENTION

Unexpectedly and surprisingly, the pyrazolotriazolopyrimidine derivative disclosed herein were found to have immune modulating efficacy in anticancer therapy. The inventors have found that the substitution of R¹ and R² in formula (Ia) or (Ib) has significantly improved the activity of the compounds as A2A receptor antagonist compared with the compound wherein R¹ and R² are both hydrogen.

In the first embodiment, disclosed herein are pyrazolotriazolopyrimidine derivatives of Formula (Ia) or (Ib). The first embodiment comprises the following aspects:

Aspect 1. A compound of Formula (Ia) or Formula (Ib)

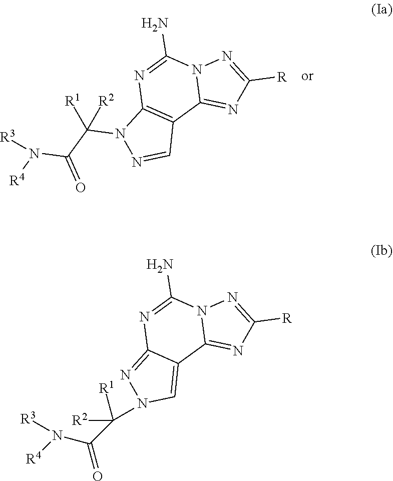

or a stereoisomer thereof, or a pharmaceutically acceptable salt thereof,
wherein:
R is a aryl group or a 5 or 6-membered heteroaryl group containing 1 or 2 heteroatoms independently selected from nitrogen, oxygen or optionally oxidized sulfur as ring member(s), and said ring is optionally substituted with at least one substituent R⁸; R¹ and R², which may be the same or different, are each independently selected from hydrogen, —C₁₋₆alkyl, —C₂₋₆alkenyl, —C₂₋₆alkynyl, —C₃₋₈cycloalkyl, heterocyclyl, aryl, or heteroaryl, wherein said C₁₋₆alkyl, —C₂₋₆alkenyl, —C₂₋₆alkynyl, —C₃₋₈cycloalkyl, heterocyclyl, aryl, or heteroaryl are each independently optionally substituted with at least one substituent R⁸, provided that at least one of R¹ and R² is not hydrogen; or
R¹ and R², together with the carbon atom to which they are attached, form a 3- to 12-membered saturated, partially or fully unsaturated ring comprising 0, 1 or 2 heteroatoms independently selected from nitrogen, oxygen or optionally oxidized sulfur as ring member(s), and said ring is optionally substituted with at least one substituent R⁸;
R³ and R⁴, together with the nitrogen atom to they are attached, form a 3- to 12-membered ring, said ring comprising 0, 1 or 2 additional heteroatoms independently selected from nitrogen, oxygen or optionally oxidized sulfur as ring member(s), said ring is optionally substituted with one or two or three substituents R⁵;
   R⁵ is independently hydrogen, halogen, —C₁₋₆alkyl, —C₂₋₆alkenyl, —C₂₋₆alkynyl, C₃₋₈cycloalkyl, heterocyclyl, aryl, heteroaryl, —CN, —NO₂, oxo, —OR^{5a}, —SO₂R^{5a}, —COR^{5a}, —CO₂R^{5a}, —CONR^{5a}R^{5b}, —C(═NR^{5a})NR^{5b}R^{5c}, —NR^{5a}R^{5b}, —NR^{5a}COR^{5b}, —NR^{5a}CONR^{5b}R^{5c}, —NR^{5a}CO₂R^{5b}, —NR^{5a}SONR^{5b}R^{5c}, —NR^{5a}SO₂NR^{5b}R^{5c}, or —NR^{5a}SO₂R^{5b}, wherein, as R⁵, each of said —C₁₋₆alkyl, —C₂₋₆alkenyl, —C₂₋₆alkynyl, C₃₋₈cycloalkyl, heterocyclyl, aryl, or heteroaryl is independently and optionally substituted with one or two or three substituents R⁶
      R^{5a}, R^{5b}, and R^{5c}, which may be the same or different, are each independently hydrogen, —C₁₋₆alkyl, —C₂₋₆alkenyl, —C₂₋₆alkynyl, cycloalkyl, heterocyclyl, aryl, or heteroaryl, wherein said —C₁₋₆alkyl, —C₂₋₆alkenyl, —C₂₋₆alkynyl, cycloalkyl, heterocyclyl, aryl, or heteroaryl are each independently optionally substituted with one or two substituent R^{5d};
         R^{5d} is independently hydrogen, halogen, cyano, —C₁₋₆alkyl, —C₂₋₆alkenyl, —C₂₋₆alkynyl, haloC₁₋₆alkyl, haloC₂₋₆alkenyl, haloC₂₋₆alkynyl, —C₁₋₆alkoxy, C₁₋₆alkoxy-C₁₋₆alkoxy-, C₂₋₆alkenyloxy-, C₂₋₆alkynyloxyl-, haloC₁₋₆alkoxy-, haloC₂₋₆alkenyloxy-, haloC₂₋₆alkynyloxy-, C₃₋₈cycloalkyloxy-, cycloalkyl, heterocyclyl, heterocyclyloxy-, aryl, aryloxy-, heteroaryl or heteroaryloxy-;
      R⁶ is independently hydrogen, halogen, —C₁₋₆alkyl, —C₂₋₆alkenyl, —C₂₋₆alkynyl, C₃₋₈cycloalkyl, heterocyclyl, aryl, heteroaryl, —CN, —NO₂, oxo, —OR^{6a}, —SO₂R^{6a}, —COR^{6a}, —CO₂R^{6a}, —CONR^{6a}R^{6b}, —C(═NR^{6a})NR^{6b}R^{6c}, —NR^{6a}R^{6b}, —NR^{6a}COR^{6b}, —NR^{6a}CONR^{6b}R^{6c}, —NR^{6a}CO₂R^{6b}, —NR^{6a}SONR^{6b}R^{6c}, —NR^{6a}SO₂NR^{6b}R^{6c}, or —NR^{6a}SO₂R^{6b};
         R^{6a}, R^{6b}, and R^{6c}, which may be the same or different, are each independently hydrogen, halogen, —C₁₋₆alkyl, —C₂₋₆alkenyl, —C₂₋₆alkynyl, —C₃₋₈cycloalkyl, heterocyclyl, aryl, or heteroaryl, wherein said C₁₋₆alkyl, —C₂₋₆alkenyl, —C₂₋₆alkynyl, cycloalkyl, heterocyclyl, aryl, or heteroaryl are each independently optionally substituted with one or two or three substituent R⁷; or
      (R^{6a} and R^{6b}), and/or (R^{6b} and R^{6c}), and/or (R^{6c} and R^{6a}), together with the atom(s) to which they are attached, form a 3- to 12-membered saturated, partially or fully unsaturated ring comprising 0, 1 or 2 additional heteroatoms independently selected from nitrogen, oxygen or optionally oxidized sulfur as ring member(s), and said ring is optionally substituted with at least one substituent R⁸; R⁷ is independently hydrogen, halogen, —C₁₋₆alkyl, —C₂₋₆alkenyl, —C₂₋₆alkynyl, C₃₋₈cycloalkyl, heterocyclyl, aryl, heteroaryl, —CN, —NO₂, oxo, —OR^{7a}, —SO₂R^{7a}, —COR^{7a}, —CO₂R^{7a}, —CONR^{7a}R^{7b}, —C(═NR^{7a})NR^{7b}R^{7c}, —NR^{7a}R^{7b}, —NR^{7a}COR^{7b}, —NR^{7a}CONR^{7b}R^{7c}, —NR^{7a}CO₂R^{7b}, —NR^{7a}SONR^{7b}R^{7c}, —NR^{7a}SO₂NR^{7b}R^{7c}, or —NR^{7a}SO₂R^{7b}, wherein said —C₁₋₆alkyl, —C₂₋₆alkenyl, —C₂₋₆alkynyl, C₃₋₈cycloalkyl, heterocyclyl, aryl, or heteroaryl are each independently optionally substituted with one or two substituents selected from halogen, hydroxy, —C₁₋₆alkyl, —C₁₋₆alkoxy, oxo, cyano, and amino;
         R^{7a}, R^{7b}, and R^{7c} each is independently hydrogen, —C₁₋₆alkyl, C₁₋₆alkoxy-C₁₋₆alkyl-, —C₂₋₆alkenyl, —C₂₋₆alkynyl, C₃₋₈cycloalkyl, heterocyclyl, aryl, or heteroaryl; and R₈ is independently hydrogen, halogen, cyano, oxo, amino, —C₁₋₆alkyl, —C₂₋₆alkenyl, —C₂₋₆alkynyl, haloC₁₋₆alkyl, haloC₂₋₆alkenyl, haloC₂₋₆alkynyl, —C₁₋₆alkoxy, C₃₋₈cycloalkyloxy, cycloalkyl, heterocyclyl, aryl, or heteroaryl.

Aspect 2: The compound according to Aspect 1, wherein R is a C-linked 5 or 6-membered heteroaryl group containing 1 or 2 heteroatoms independently selected from nitrogen, oxygen or optionally oxidized sulfur as ring member(s).

Aspect 3: The compound according to Aspect 5, wherein R is furanyl, pyrazinyl or thiazolyl; preferably, furan-2-yl, 3-methylpyrazin-2-yl or thiazol-2-yl.

Aspect 4: The compound according to Aspect 1, wherein R1 is hydrogen or C1-6alkyl, preferably hydrogen, methyl, ethyl; more preferably hydrogen; and R2 is C1-6alkyl (preferably methyl, isopropyl, ethyl, propyl, butyl, or isobutyl) optionally substituted with phenyl or —C1-6alkoxy (preferably methoxy); aryl (i.e., phenyl or naphthyl) optionally substituted with halogen or C1-6alkoxy (e.g., phenyl, 2-fluorophenyl, 3-fluorophenyl, 4-fluorophenyl, 4-methoxylphenyl, 2-methoxylphenyl, 3-methoxylphenyl, 4-trifluoromethylphenyl, 3,4-difluorophenyl); —C3-8cycloalkyl (preferably cyclopropyl), or heterocyclyl (preferably, 4- to 7-membered monocyclic saturated heterocyclyl comprising one heteroatom selected from oxygen, nitrogen and optionally oxidized sulfur as ring member).

Aspect 5: The compound according to any one of Aspects 1-4, wherein R3 and R4, together with the nitrogen atom to they are attached, form a 3-, 4-, 5-, 6-, 7-, 8-, or 9-membered monocyclic ring comprising 0, 1 or 2 additional heteroatoms independently selected from nitrogen, oxygen or optionally oxidized sulfur as ring member(s), said ring is optionally substituted with one or two or three substituents R5.

Aspect 6: The compound according to Aspect 5, wherein R3 and R4, together with the nitrogen atom to they are attached, form a 3-, 4-, 5-, 6-, 7-, 8-, or 9-membered monocyclic ring comprising 0 additional heteroatom independently selected from nitrogen, oxygen or optionally oxidized sulfur as ring member(s), said ring is optionally substituted with one or two or three substituents R5.

Aspect 7: The compound according to Aspect 5, wherein R3 and R4, together with the nitrogen atom to they are attached, form a 3-, 4-, 5-, 6-, 7-, 8-, or 9-membered monocyclic ring comprising 1 additional heteroatom independently selected from nitrogen, oxygen or optionally oxidized sulfur as ring member(s), said ring is optionally substituted with one or two or three substituents R5.

Aspect 8: The compound according to Aspect 7, wherein R3 and R4, together with the nitrogen atom to they are attached, form a 3-, 4-, 5-, 6-, 7-, 8-, or 9-membered monocyclic ring comprising one additional nitrogen heteroatom as ring member, said ring is optionally substituted with one or two or three substituents R5.

Aspect 9: The compound according to any one of Aspects 5-9, wherein said ring is saturated.

Aspect 10: The compound according to Aspect 5, wherein R3 and R4, together with the nitrogen atom to they are attached, form an azetidinyl

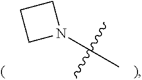 [SMILES: *N1CCC1]

pyrrolidinyl

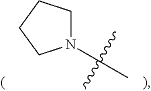 [SMILES: *N1CCCC1]

(or piperidinyl

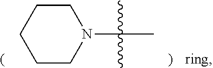 [SMILES: *N1CCCCC1]

each of which is optionally substituted with R5 as defined with Formula (Ia) or (Ib).

Aspect 11: The compound according to Aspect 5, wherein R3 and R4, together with the nitrogen atom to they are attached, form a piperazinyl ring optionally substituted with R5 as defined with Formula (Ia) or (Ib)

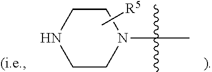

Aspect 12: The compound according to any one of Aspects 1-4, wherein R³ and R⁴, together with the nitrogen atom to they are attached, form a 7-, 8-, 9-, 10-, 11-, or 12-membered bicyclic ring comprising 0, 1 or 2 additional heteroatoms independently selected from nitrogen, oxygen or optionally oxidized sulfur as ring member(s), said ring is optionally substituted with one or two or three substituents R⁵.

Aspect 13: The compound according to Aspect 12, wherein R³ and R⁴, together with the nitrogen atom to they are attached, form a 7-, 8-, 9-, 10-, 11-, or 12-membered bicyclic fused ring comprising 0, 1 or 2 additional heteroatoms independently selected from nitrogen, oxygen or optionally oxidized sulfur as ring member(s), said ring is optionally substituted with one or two or three substituents R⁵.

Aspect 14: The compound according to Aspect 12, wherein R³ and R⁴, together with the nitrogen atom to they are attached, form a 7-, 8-, 9-, 10-, 11-, or 12-membered bicyclic fused ring comprising 0 or 1 additional heteroatom independently selected from nitrogen, oxygen or optionally oxidized sulfur as ring member(s), said ring is optionally substituted with one or two or three substituents R⁵.

Aspect 15: The compound according to Aspect 12, wherein R³ and R⁴, together with the nitrogen atom to they are attached, form a 10-membered bicyclic fused ring comprising 0 or 1 additional nitrogen heteroatom as ring member, said ring is optionally substituted with one or two or three substituents R⁵.

Aspect 16: The compound according to Aspect 15, wherein R³ and R⁴, together with the nitrogen atom to they are attached, form a

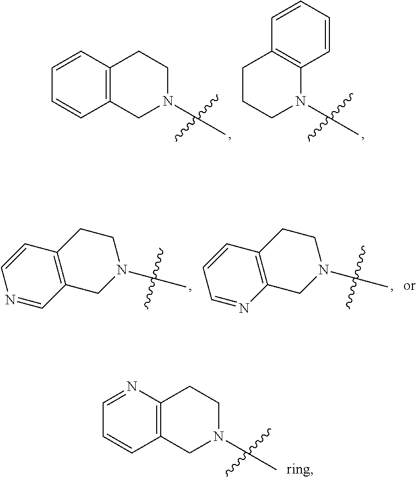 [SMILES: *N1CCc2ccccc2C1 ; *N1CCCc2ccccc21 ; *N1CCc2ccncc2C1 ; *N1CCc2cccnc2C1 ; *N1CCc2ncccc2C1]

each of said ring is optionally substituted with one or two or three substituents R⁵.

Aspect 17: The compound according to Aspect 12, wherein R³ and R⁴, together with the nitrogen atom to they are attached, form a 7-, 8-, 9-, 10-, 11-, or 12-membered bicyclic spiro ring comprising 0, 1 or 2 additional heteroatoms independently selected from nitrogen, oxygen or optionally oxidized sulfur as ring member(s), said ring is optionally substituted with one or two or three substituents R⁵.

Aspect 18: The compound according to Aspect 17, wherein R³ and R⁴, together with the nitrogen atom to they are attached, form azaspiro[3.3]heptane, azaspiro[3.5]nonane, azaspiro[3.4]octane, azaspiro[5.5]undecane, or azaspiro[4.5]decane, each of which comprises 0 or 1 additional nitrogen or oxygen atom as ring member, and said ring is optionally substituted with one or two or three substituents R⁵.

Aspect 19: The compound according to Aspect 18, wherein R³ and R⁴, together with the nitrogen atom to they are attached, form a

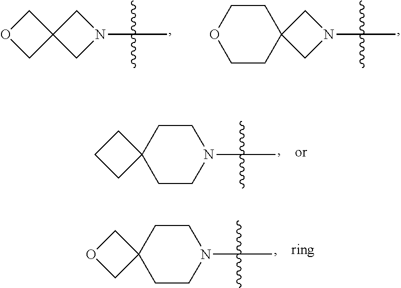

and said ring is optionally substituted with one or two or three substituents R⁵.

Aspect 20: The compound according to any one of Aspects 5-19, wherein said ring is substituted with one R⁵.

Aspect 21: The compound according to any one of Aspects 5-19, wherein said ring is substituted with two R⁵.

Aspect 22: The compound according to Aspect 21, wherein said ring is substituted with one C₁₋₆alkyl (preferably methyl) and further substituted with one R⁵.

Aspect 23: The compound according to any one of Aspects 20-22, wherein R⁵ is halogen, —C₁₋₆alkyl, aryl, —OR^{5a}, or —CO₂R^{5a}, wherein R^{5a} is as defined with Formula (Ia) or (Ib).

Aspect 24: The compound according to Aspect 23, wherein R⁵ is —CO₂R^{5a}, wherein R^{5a} is —C₁₋₆alkyl, preferably methyl.

Aspect 25: The compound according to Aspect 23, wherein R⁵ is —OR^{5a}, wherein R^{5a} is —C₁₋₆alkyl, optionally substituted with one R^{5d}, wherein R^{5d} is hydrogen, halogen (preferably fluoro), or —C₁₋₆alkoxy.

Aspect 26: The compound according to Aspect 23, wherein R⁵ is —OR^{5a}, wherein R^{5a} is trifluoromethoxy, methoxy, methoxyethoxy, or hydroxy.

Aspect 27: The compound according to Aspect 23, wherein R⁵ is phenyl, optionally substituted with one or two or three substituents R⁶, wherein R⁶ is defined as with Formula (Ia) or (Ib).

Aspect 28: The compound according to Aspect 27, wherein R⁵ is phenyl, optionally substituted with one or two or three substituents R⁶, wherein R⁶ is independently halogen (preferably fluoro), —OR^{6a}, or NR^{6a}R^{6b}C(O)—, wherein R^{6a} and R^{6b} are defined as with Formula (Ia) or (Ib).

Aspect 29: The compound according to Aspect 28, wherein R⁵ is phenyl, optionally substituted with one substituent R⁶, wherein
R⁶ is NR^{6a}R^{6b}C(O)—, wherein
   R^{6a} and R^{6b} are each independently hydrogen, —C₁₋₆alkyl, or —C₃₋₈cycloalkyl, said —C₁₋₆alkyl and —C₃₋₈cycloalkyl are each optionally substituted with one R⁷, wherein
      R⁷ is heterocyclyl (preferably 3- to 8-membered heterocyclic comprising one or two heteroatoms independently selected from nitrogen, oxygen or optionally oxidized sulfur as ring member(s), optionally substituted with hydroxy, —C₁₋₆alkyl, or —C₁₋₆alkoxy.

Aspect 30: The compound according to Aspect 28, wherein R⁵ is phenyl, optionally substituted with one substituent R⁶, wherein
R⁶ is NR^{6a}R^{6b}C(O)—, wherein
   R^{6a} and R^{6b} are each independently hydrogen, —C₁₋₆alkyl, or —C₃₋₈cycloalkyl, said —C₁₋₆alkyl is optionally substituted with one R⁷, wherein
      R⁷ is 3- to 8-membered saturated monocyclic heterocyclic comprising one heteroatom selected from nitrogen or oxygen as ring member (preferably oxetanyl), optionally substituted with hydroxy.

Aspect 31: The compound according to Aspect 28, wherein R⁵ is phenyl, optionally substituted with one substituent R⁶, wherein
R⁶ is NR^{6a}R^{6b}C(O)—, wherein
   R^{6a} and R^{6b}, together with the nitrogen atom to which they are attached, form a 3- to 12-membered saturated ring comprising 0, 1 or 2 additional heteroatoms independently selected from nitrogen, oxygen or optionally oxidized sulfur as ring member(s).

Aspect 32: The compound according to Aspect 31, wherein R^{6a} and R^{6b}, together with the nitrogen atom to which they are attached, form a 3- to 8-membered saturated monocyclic ring comprising 0 or 1 additional heteroatom independently selected from nitrogen or oxygen as ring member.

Aspect 33: The compound according to Aspect 32, wherein R^{6a} and R^{6b}, together with the nitrogen atom to which they are attached, form a 6-membered saturated monocyclic ring comprising 0 or 1 additional nitrogen heteroatom as ring member.

Aspect 34: The compound according to Aspect 31, wherein R^{6a} and R^{6b}, together with the nitrogen atom to which they are attached, form a piperidinyl ring.

Aspect 35: The compound according to Aspect 28, wherein R⁵ is phenyl, optionally substituted with one or two halogen and further optionally substituted with one substituent R⁶, wherein
R⁶ is —OR^{6a}, wherein
   R^{6a} is —C₁₋₆alkyl optionally substituted with one R⁷, wherein
      R⁷ is heterocyclyl, —OR^{7a}, or —NR^{7a}R^{7b}, wherein
         R^{7a} and R^{b} are each independently hydrogen, —C₁₋₆alkyl, C₁₋₆alkoxy-C₁₋₆alkyl-; and
         Said heterocyclyl is optionally substituted with halogen, hydroxy, or —C₁₋₆alkyl.

Aspect 36: The compound according to Aspect 35, wherein said heterocyclyl as R⁷ is a 4-, 5-, 6-, 7- or 8-membered heterocyclyl comprising one or two heteroatoms independently selected from nitrogen, oxygen or optionally oxidized sulfur as ring member(s), optionally substituted with —C₁₋₆alkyl; preferably 5- or 6-membered heterocyclyl comprising one or two heteroatoms independently selected from nitrogen, oxygen or optionally oxidized sulfur as ring member(s).

Aspect 37: The compound according to Aspect 36, wherein R⁷ is morpholinyl, morpholino, pyrrolidinyl, pyrrolidino, 4-methylpiperizinyl, or piperidinyl.

Aspect 38: The compound according to Aspect 35, wherein R⁶ is —OR^{6a}, wherein R^{6a} is —C₁₋₆alkyl optionally substituted with one R⁷, wherein R⁷ is —OR^{7a}, wherein R^{7a} is hydrogen, —C₁₋₆alkyl, C₁₋₆alkoxy-C₁₋₆alkyl-.

Aspect 39: The compound according to Aspect 35, wherein R⁶ is —OR^{6a}, wherein R^{6a} is —C₁₋₆alkyl optionally substituted with one R⁷, wherein R⁷ is —NR^{7a}R^{7b}, wherein R^{7a} and R^{7b} are hydrogen, or —C₁₋₆alkyl.

Aspect 40: The compound according to Aspect 35, wherein R⁶ is methoxyethoxy-, methoxyethoxyethoxy-, 2-hydroxyethoxy, 2-hydroxypropoxy-, aminoethoxy-, N,N-dimethylaminoethoxy-, or N-methylaminoethoxy-.

Aspect 41: The compound of Aspect 1, which is Compound Nos. A1, A2, A3, A4, A5, A6, A7, A8, A9, A10, A11, A12, A13, A14, A15, A16, A17, A18, A19, A20, A21, A22, A23, A24, A25, A26, A27, A28, A29, A30, A31, A32, A33, A34, A35, A36, A37, A38, A39, A40, A41, A42, A43, A44, A45, A46, A47, A48, A49, A50, A51, A52, A53, A54, A55, A56, A57, A58, A59, A60, A61, A62, A63, A64, A65, A66, A67, A68, A69, A70, A71, A72, A73, A74, A75, A76, A77, A78, A79, A80, A81, A82, A83, A84, A85, A86, A87, A88, A89, A90, A91, A92, A93, A94, A95, A96, A97, A98, A99, A100, A101, A102, A103, A104, A105, A106, A107, A108, B1, B2, B3, B4, B5, B6, B7, B8, B9, B10, B11, B12, B13, B14, B15, B16, B17, B18, B19, B20, B21, B22, B23, B24, B25, B26, B27, B28, B29, B30, B31, B32, B33, B34, C1, C2, C3, C4, C5, C6, C7, C8, C9, C10, C11, C12, C13, C14, C15, C16, C17, C18, C19, C20, C21, C22, C23, C24, C25, D1, or D2.

In the second embodiment, disclosed herein is a pharmaceutical composition comprising the compound of any of Aspects 1-41 or a stereoisomer thereof, or a pharmaceutically acceptable salt thereof and a pharmaceutically acceptable excipient.

In the third embodiment, disclosed herein is a method of treating cancer, comprising administering a subject in need thereof the compound of any of Aspects 1-41 or a stereoisomer thereof, or a pharmaceutically acceptable salt thereof.

### DETAILED DESCRIPTION OF THE INVENTION

The following terms have the indicated meanings throughout the specification:

As used herein, including the appended claims, the singular forms of words such as “a”, “an”, and “the”, include their corresponding plural references unless the context clearly dictates otherwise.

The term “or” is used to mean, and is used interchangeably with, the term “and/or” unless the context clearly dictates otherwise.

The term “alkyl” herein refers to a hydrocarbon group selected from linear and branched saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “halogen” herein refers to fluoro (F), chloro (Cl), bromo (Br) and iodo (I).

The term “haloalkyl” herein refers to an alkyl group in which one or more hydrogen is/are replaced by one or more halogen atoms such as fluoro, chloro, bromo, and iodo. Examples of the haloalkyl include haloC₁₋₈alkyl, haloC₁₋₆alkyl or halo C₁₋₄alkyl, but not limited to —CF₃, —CH₂Cl, —CH₂CF₃, —CCl₂, CF₃, and the like.

The term “alkenyl” herein refers to a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkynyl” herein refers to a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “alkyloxy” or “alkoxy” herein refers to an alkyl group as defined above attached to the parent molecular moiety through an oxygen atom. Examples of an alkyloxy, e.g., C₁₋₆alkyloxy or C₁₋₄ alkyloxy includes, but not limited to, methoxy, ethoxy, isopropoxy, propoxy, n-butoxy, tert-butoxy, pentoxy and hexoxy and the like.

The term “alkoxy-alkyl-” refers to an alkyl group as defined above further substituted with an alkoxy as defined above. Examples of an alkoxy-alkyl-, e.g., C₁₋₈alkoxy-C₁₋₈alkyl- or C₁₋₆alkoxy-C₁₋₆alkyl- includes, but not limited to, methoxymethyl, ethoxymethyl, ethoxyethyl, isopropoxymethyl, or propoxymethyl and the like.

The term “cycloalkyl” herein refers to a hydrocarbon group selected from saturated and partially unsaturated cyclic hydrocarbon groups, comprising monocyclic and polycyclic (e.g., bicyclic and tricyclic) groups. For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. In particular, Examples of the saturated monocyclic cycloalkyl group, e.g., C₃₋₈ cycloalkyl, include, but not limited to cyclopropyl, cyclobutyl, cyclopentyl, cyclohexyl, cycloheptyl, and cyclooctyl groups. In a preferred embedment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems, such as

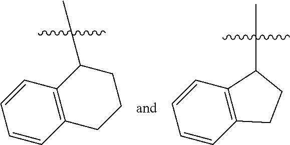 [SMILES: *C1CCCc2ccccc21 ; *C1CCc2ccccc21]

wherein the wavy lines indicate the points of attachment. The ring may be saturated or have at least one double bond (i.e. partially unsaturated), but is not fully conjugated, and is not aromatic, as aromatic is defined herein.

The term “aryl” used alone or in combination with other terms refers to a group selected from:
5- and 6-membered carbocyclic aromatic rings, e.g., phenyl;
bicyclic ring systems such as 7 to 12 membered bicyclic ring systems, wherein at least one ring is carbocyclic and aromatic, e.g., naphthyl and indanyl; and,
tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeable throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring includes, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

The term “heteroaryl” herein refers to a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
8- to 12-membered bicyclic rings comprising at least one heteroatom, for example, from 1 to 4, or, in some embodiments, from 1 to 3, or, in other embodiments, 1 or 2, heteroatoms, selected from nitrogen, oxygen or optionally oxidized sulfur as ring member(s), with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from nitrogen, oxygen or optionally oxidized sulfur as ring member(s), with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

The term “C-linked heteroaryl” as used herein means that the heteroaryl group is connected to the core molecule by a bond from a C-atom of the heteroaryl ring.

The term “optionally oxidized sulfur” used herein refer to S, SO or SO₂.

The terms “aromatic heterocyclic ring” and “heteroaryl” are used interchangeable throughout the disclosure herein. In some embodiments, a monocyclic or bicyclic aromatic heterocyclic ring has 5-, 6-, 7-, 8-, 9- or 10-ring forming members with 1, 2, 3, or 4 heteroatom ring members independently selected from nitrogen (N), sulfur (S) and oxygen (O) and the remaining ring members being carbon. In some embodiments, the monocyclic or bicyclic aromatic heterocyclic ring is a monocyclic or bicyclic ring comprising 1 or 2 heteroatom ring members independently selected from nitrogen (N), sulfur (S) and oxygen (O). In some embodiments, the monocyclic or bicyclic aromatic heterocyclic ring is a 5- to 6-membered heteroaryl ring, which is monocyclic and which has 1 or 2 heteroatom ring members independently selected from nitrogen (N), sulfur (S) and oxygen (O). In some embodiments, the monocyclic or bicyclic aromatic heterocyclic ring is a 8- to 10-membered heteroaryl ring, which is bicyclic and which has 1 or 2 heteroatom ring members independently selected from nitrogen, sulfur and oxygen.

Examples of the heteroaryl group or the monocyclic or bicyclic aromatic heterocyclic ring include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl (such as 1,2,3-thiadiazolyl, 1,2,4-thiadiazolyl, or 1,3,4-thiadiazolyl), tetrazolyl, thienyl (such as thien-2-yl, thien-3-yl), triazinyl, benzothienyl, furyl or furanyl, benzofuryl, benzoimidazolyl, indolyl, isoindolyl, indolinyl, oxadiazolyl (such as 1,2,3-oxadiazolyl, 1,2,4-oxadiazolyl, or 1,3,4-oxadiazolyl), phthalazinyl, pyrazinyl, pyridazinyl, pyrrolyl, triazolyl (such as 1,2,3-triazolyl, 1,2,4-triazolyl, or 1,3,4-triazolyl), quinolinyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl (such as furazan-2-yl, furazan-3-yl), benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinoline.

“Heterocyclyl”, “heterocycle” or “heterocyclic” are interchangeable and refer to a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups. The term “optionally oxidized sulfur” used herein refer to S, SO or SO₂.

The term “monocyclic heterocyclyl” refers to monocyclic groups in which at least one ring member is a heteroatom selected from nitrogen, oxygen or optionally oxidized sulfur. A heterocycle may be saturated or partially saturated.

Exemplary monocyclic 4 to 9-membered heterocyclyl groups include, but not limited to, (as numbered from the linkage position assigned priority 1) pyrrolidin-1-yl, pyrrolidin-2-yl, pyrrolidin-3-yl, imidazolidin-2-yl, imidazolidin-4-yl, pyrazolidin-2-yl, pyrazolidin-3-yl, piperidin-1-yl, piperidin-2-yl, piperidin-3-yl, piperidin-4-yl, 2,5-piperazinyl, pyranyl, morpholinyl, morpholino, morpholin-2-yl, morpholin-3-yl, oxiranyl, aziridin-1-yl, aziridin-2-yl, azocan-1-yl, azocan-2-yl, azocan-3-yl, azocan-4-yl, azocan-5-yl, thiiranyl, azetidin-1-yl, azetidin-2-yl, azetidin-3-yl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepan-1-yl, azepan-2-yl, azepan-3-yl, azepan-4-yl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepanyl, 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinyl, imidazolinyl, pyrimidinonyl, or 1,1-dioxo-thiomorpholinyl.

The term “spiro heterocyclyl” refers to a 5 to 20-membered polycyclic heterocyclyl with rings connected through one common carbon atom (called a spiro atom), comprising one or more heteroatoms selected from nitrogen, oxygen or optionally oxidized sulfur as ring members, with the remaining ring members being carbon. One or more rings of a spiro heterocyclyl group may contain one or more double bonds, but none of the rings has a completely conjugated pi-electron system. Preferably a spiro heterocyclyl is 6 to 14-membered, and more preferably 7 to 12-membered. According to the number of common spiro atoms, a spiro heterocyclyl is divided into mono-spiro heterocyclyl, di-spiro heterocyclyl, or poly-spiro heterocyclyl, and preferably refers to mono-spiro heterocyclyl or di-spiro heterocyclyl, and more preferably 4-membered/4-membered, 3-membered/5-membered, 4-membered/5-membered, 4-membered/6-membered, 5-membered/5-membered, or 5-membered/6-membered mono-spiro heterocyclyl. Representative examples of spiro heterocyclyls include, but not limited to the following groups: 2,3-dihydrospiro[indene-1,2′-pyrrolidine] (e.g., 2,3-dihydrospiro[indene-1,2′-pyrrolidine]-1′-yl), 1,3-dihydrospiro[indene-2,2′-pyrrolidine] (e.g., 1,3-dihydrospiro[indene-2,2′-pyrrolidine]-1′-yl), azaspiro[2.4]heptane (e.g., 5-azaspiro[2.4]heptane-5-yl), azaspiro[3.4]octane (e.g., 6-azaspiro[3.4]octane-6-yl), 2-oxa-6-azaspiro[3.4]octane (e.g., 2-oxa-6-azaspiro[3.4]octane-6-yl), azaspiro[3.4]octane (e.g., 6-azaspiro[3.4]octan-6-yl), azaspiro[3.4]octane (e.g., 6-azaspiro[3.4]octan-6-yl), 1,7-dioxaspiro[4.5]decane, 2-oxa-7-aza-spiro[4.4]nonane (e.g., 2-oxa-7-aza-spiro[4.4]non-7-yl), 7-oxa-spiro[3.5]nonyl and 5-oxa-spiro[2.4]heptyl.

The term “fused heterocyclic group” refers to a 5 to 20-membered polycyclic heterocyclyl group, wherein each ring in the system shares an adjacent pair of atoms (carbon and carbon atoms or carbon and nitrogen atoms) with another ring, comprising one or more heteroatoms selected from nitrogen, oxygen or optionally oxidized sulfur as ring members, with the remaining ring members being carbon. One or more rings of a fused heterocyclic group may contain one or more double bonds, but none of the rings has a completely conjugated pi-electron system. Preferably, a fused heterocyclyl is 6 to 14-membered, and more preferably 7 to 10-membered. According to the number of membered rings, a fused heterocyclyl is divided into bicyclic, tricyclic, tetracyclic, or polycyclic fused heterocyclyl, preferably refers to bicyclic or tricyclic fused heterocyclyl, and more preferably 5-membered/5-membered, or 5-membered/6-membered bicyclic fused heterocyclyl. Representative examples of fused heterocycles include, but not limited to, the following groups octahydrocyclopenta[c]pyrrole (e.g., octahydrocyclopenta[c]pyrrol-2-yl), octahydropyrrolo[3,4-c]pyrrolyl, octahydroisoindolyl, isoindolinyl (e.g., isoindoline-2-yl), octahydro-benzo[b][1,4]dioxin.

The term “bridged heterocyclyl” refers to a 5 to 14-membered polycyclic heterocyclic alkyl group, wherein every two rings in the system share two disconnected atoms, comprising one or more heteroatoms selected from nitrogen, oxygen or optionally oxidized sulfur as ring members, with the remaining ring members being carbon. One or more rings of a bridged heterocyclyl group may contain one or more double bonds, but none of the rings has a completely conjugated pi-electron system. Preferably, a bridged heterocyclyl is 6 to 14-membered, and more preferably 7 to 10-membered. According to the number of membered rings, a bridged heterocyclyl is divided into bicyclic, tricyclic, tetracyclic or polycyclic bridged heterocyclyl, and preferably refers to bicyclic, tricyclic or tetracyclic bridged heterocyclyl, and more preferably bicyclic or tricyclic bridged heterocyclyl. Representative examples of bridged heterocyclyls include, but not limited to, the following groups: 2-azabicyclo[2.2.1]heptyl, azabicyclo[3.1.0]hexyl, 2-azabicyclo[2.2.2]octyl and 2-azabicyclo[3.3.2]decyl.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

The term “substantially pure” as used herein means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

When compounds disclosed herein contain olefinic double bonds, unless specified otherwise, such double bonds are meant to include both E and Z geometric isomers.

When compounds disclosed herein contain a di-substituted cyclohexyl or cyclobutyl group, substituents found on cyclohexyl or cyclobutyl ring may adopt cis and trans formations. Cis formation means that both substituents are found on the upper side of the 2 substituent placements on the carbon, while trans would mean that they were on opposing sides.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

“Diastereomers” refers to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

“Pharmaceutically acceptable salts” refers to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “a pharmaceutically acceptable salt thereof” include salts of at least one compound of Formula (Ia) or (Ib), and salts of the stereoisomers of the compound of Formula (Ia) or (Ib), such as salts of enantiomers, and/or salts of diastereomers.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer thereof, and/or at least one pharmaceutically acceptable salt thereof disclosed herein effective to “treat” as defined above, a disease or disorder in a subject. In the case of combination therapy, the “therapeutically effective amount” refers to the total amount of the combination objects for the effective treatment of a disease, a disorder or a condition.

The pharmaceutical composition comprising the compound disclosed herein can be administrated via oral, inhalation, rectal, parenteral or topical administration to a subject in need thereof. For oral administration, the pharmaceutical composition may be a regular solid Formulation such as tablets, powder, granule, capsules and the like, a liquid Formulation such as water or oil suspension or other liquid Formulation such as syrup, solution, suspension or the like; for parenteral administration, the pharmaceutical composition may be solution, water solution, oil suspension concentrate, lyophilized powder or the like. Preferably, the Formulation of the pharmaceutical composition is selected from tablet, coated tablet, capsule, suppository, nasal spray or injection, more preferably tablet or capsule. The pharmaceutical composition can be a single unit administration with an accurate dosage. In addition, the pharmaceutical composition may further comprise additional active ingredients.

All Formulations of the pharmaceutical composition disclosed herein can be produced by conventional methods in the pharmaceutical field. For example, the active ingredient can be mixed with one or more excipients, then to make the desired Formulation. A “pharmaceutically acceptable excipient” refers to conventional pharmaceutical carriers suitable for the desired pharmaceutical Formulation, for example: a diluent, a vehicle such as water, various organic solvents, etc, a filler such as starch, sucrose, etc a binder such as cellulose derivatives, alginates, gelatin and polyvinylpyrrolidone (PVP); a wetting agent such as glycerol; a disintegrating agent such as agar, calcium carbonate and sodium bicarbonate; an absorption enhancer such as quaternary ammonium compound; a surfactant such as hexadecanol; an absorption carrier such as Kaolin and soap clay; a lubricant such as talc, calcium stearate, magnesium stearate, polyethylene glycol, etc. In addition, the pharmaceutical composition further comprises other pharmaceutically acceptable excipients such as a decentralized agent, a stabilizer, a thickener, a complexing agent, a buffering agent, a permeation enhancer, a polymer, aromatics, a sweetener, and a dye.

The term “disease” refers to any disease, discomfort, illness, symptoms or indications, and can be interchangeable with the term “disorder” or “condition”.

Throughout this specification and the claims which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cₙ₋ₘ” indicates a range which includes the endpoints, wherein n and m are integers and indicate the number of carbons. Examples include C₁₋₈, C₁₋₆, and the like.

Unless specifically defined elsewhere in this document, all other technical and scientific terms used herein have the meaning commonly understood by one of ordinary skill in the art to which this invention belongs.

### General Synthesis

Compounds disclosed herein, including salts thereof, can be prepared using known organic synthesis techniques and can be synthesized according to any of numerous possible synthetic routes.

The reaction for preparing compounds disclosed herein can be carried out in suitable solvents which can be readily selected by one of skill in the art of organic synthesis. Suitable solvents can be substantially non-reactive with the starting materials, the intermediates, or products at the temperatures at which the reactions are carried out, e.g., temperatures which can range from room temperature to the solvent's boiling temperature. A given reaction can be carried out in one solvent or mixture of solvents.

The selection of appropriate protecting group, can be readily determined by one skilled in the art.

Reactions can be monitored according to any suitable method known in the art, such as NMR, UV, HPLC, LC-MS and TLC. Compounds can be purified by a variety of methods, including HPLC and normal phase silica chromatography.

Chiral analytic HPLC was used for the retention time analysis of different chiral examples, the conditions were divided into the methods as below according to the column, mobile phase, solvent ration used.

The compounds disclosed herein can be prepared by following Scheme I and Scheme II.

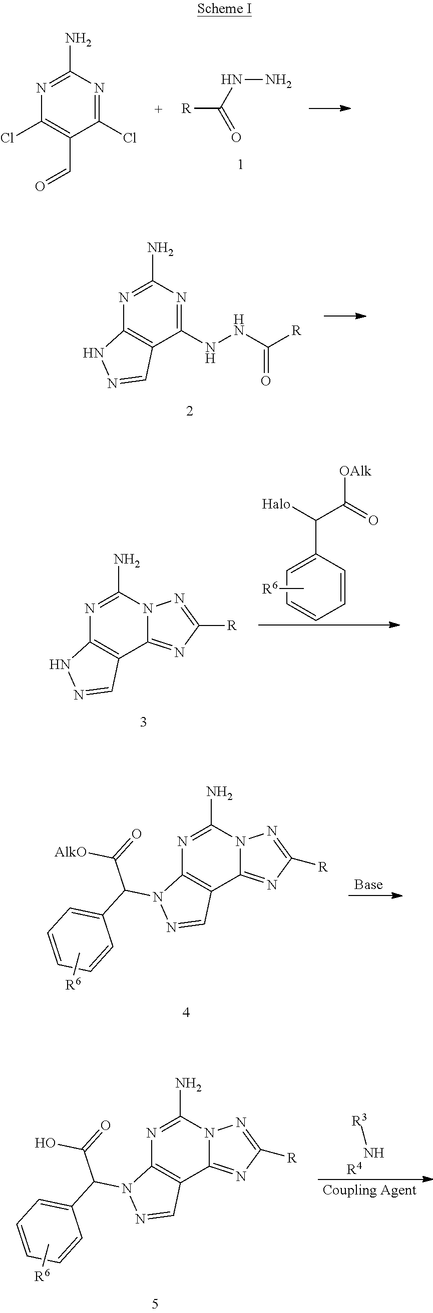

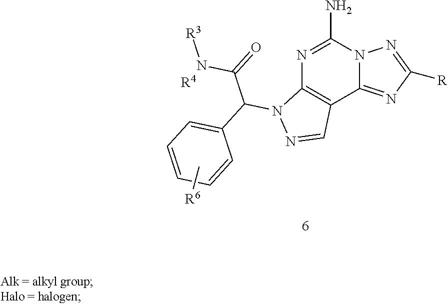

wherein R and R⁶ are as defined with formula (Ia) or (Ib).

In Scheme I, a commercially available aldehyde is reacted with carbohydrazide 1 (such as furan-2-carbohydrazide) to form formula 2, which is rearranged in the presence of acidic condition to give formula 3. Then formula 3 is reacted with 2-halo-2-phenylacetate ester (such as methyl 2-bromo-2-phenylacetate) to form formula 4 which subsequently is hydrolyzed into the free acid of formula 5 by using a base such as sodium hydroxide or potassium tert-butoxide. The further coupling of the acid 5 is accomplished under standard conditions known in the art to provide a compound of Formula 6.

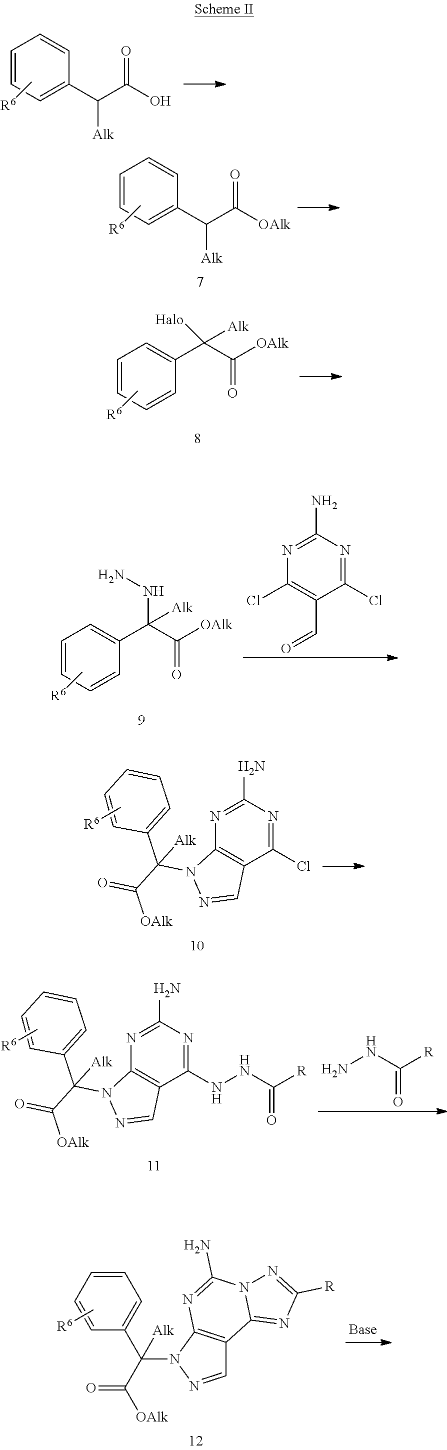

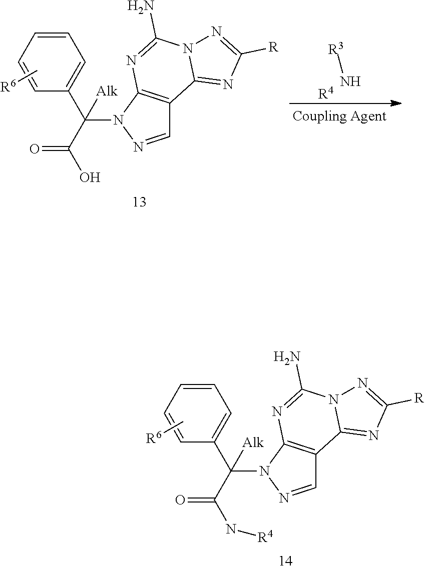

wherein R, R⁶ and R³ and R⁴ are as defined with formula (Ia) or (Ib).

In Scheme II, a commercially available acid (such as 2-phenylpropanoic acid) is esterified under standard conditions known in the art to afford one compound of formula 7 which is reacted with halogenation reagent such as N-bromosuccinimide to introduce one halo atom at the alpha position. A compound of formula 8 is reacted with hydrazine hydrate to form formula 9. Formula 9 is reacted with a commercially available 2-amino-4,6-dichloropyrimidine-5-carbaldehyde to afford formula 10, which is reacted with carbohydrazide (such as furan-2-carbohydrazide) and further rearranged in the presence of acidic condition to give formula 12. The ester 12 subsequently is hydrolyzed into the free acid of formula 13 by using a base such as sodium hydroxide or potassium tert-butoxide. The further coupling of the acid 13 is accomplished under standard conditions known in the art to provide a compound of Formula 14.

### EXAMPLES

The examples below are intended to be purely exemplary and should not be considered to be limiting in any way. Unless otherwise specified, the experimental methods in the Examples described below are conventional methods. Unless otherwise specified, the reagents and materials are all commercially available. All solvents and chemicals employed are of analytical grade or chemical purity. Solvents are all redistilled before use. Anhydrous solvents are all prepared according to standard methods or reference methods. Silica gel (100-200 meshes) for column chromatography and silica gel (GF254) for thin-layer chromatography (TLC) are commercially available from Tsingdao Haiyang Chemical Co., Ltd. or Yantai Chemical Co., Ltd. of China; all are eluted with petroleum ether (60-90° C.)/ethyl acetate (v/v), and visualized by iodine or the solution of molybdphosphoric acid in ethanol unless otherwise specified. All extraction solvents, unless otherwise specified, are dried over anhydrous Na₂SO₄. ¹H NMR spectra are recorded on Bruck-400 nuclear magnetic resonance spectrometer with TMS (tetramethylsilane) as the internal standard. LC/MS data are recorded by using Agilent1100 High Performance Liquid Chromatography-Ion Trap Mass Spectrometer (LC-MSD Trap) equipped with a diode array detector (DAD) detected at 214 nm and 254 nm, and an ion trap (ESI source). All compound names except the reagents were generated by ChemDraw® version 14.0.

In the following examples, the following abbreviations are used:
AcOH Acetic acid
Aq Aqueous
Brine Saturated aqueous sodium chloride solution
Bn Benzyl
BnBr Benzyl Bromide
BPO Benzoyl peroxide
BSA N,O-Bis(trimethylsilyl)acetamide
CH₂Cl₂ or DCM Dichloromethane
DMF N,N-Dimethylformamide
Dppf 1,1′-bis(diphenylphosphino)ferrocene
DBU 1,8-diazabicyclo[5.4.0]undec-7-ene
DIEA or DIPEA N,N-diisopropylethylamine
DMAP 4-N,N-dimethylaminopyridine
DMF N,N-dimethylformamide
DMSO Dimethyl sulfoxide
EtOAc Ethyl acetate
EtOH Ethanol
Et₂O or ether Diethyl ether
g Grams
h or hr Hour
HATU O-(7-Azabenzotriazol-1-yl)-N,N,N′,N′-tetramethyluronium hexafluorophosphate
Hex Hexane
HCl Hydrochloric acid
HMDS Hexamethyldisilazane
HPLC High-performance liquid chromatography
IPA Isopropyl alcohol
i-PrOH Isopropyl alcohol
LCMS Liquid chromatography-mass spectrometry
mg milligrams
mL milliliters
mmol millimole
MeCN Acetonitrile
MeOH Methanol
Min minutes
ms or MS Mass spectrum
Na₂SO₄ Sodium sulfate
NBS N-Bromosuccinimide
PE petroleum ether
prep preparative
Rt Retention time
Rt or rt Room temperature
TBAF Tetra-butyl ammonium fluoride
TBSCl tert-Butyldimethylsilyl chloride
TFA Trifluoroacetic acid
THF tetrahydrofuran
TLC thin layer chromatography
μL microliters

### Compound A1: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)propan-1-one

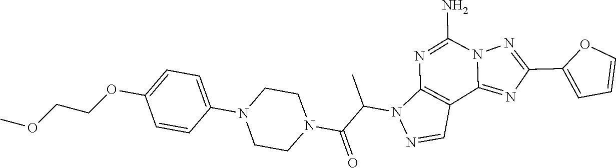 [SMILES: COCCOc1ccc(N2CCN(C(=O)C(C)n3ncc4c3nc(N)n3nc(-c5ccco5)nc43)CC2)cc1]

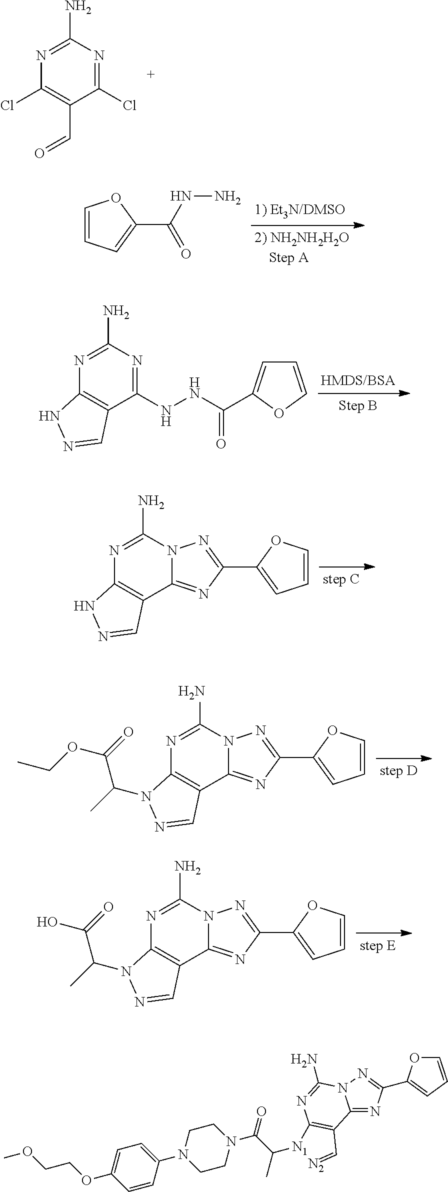

### Step A: N′-(6-amino-1H-pyrazolo[3,4-d]pyrimidin-4-yl)furan-2-carbohydrazide

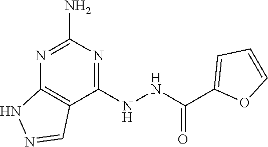 [SMILES: Nc1nc(NNC(=O)c2ccco2)c2cn[nH]c2n1]

To a stirred solution of 2-amino-4,6-dichloropyrimidine-5-carbaldehyde (50 g, 0.26 mol) and Et₃N (28.9 g, 0.28 mol) in DMSO (600 mL) was added furan-2-carbohydrazide (32.8 g, 0.26 mol) in portionwise. Then the reaction mixture was stirred at rt overnight. LCMS showed the starting materials were converted into the intermediate. NH₂NH₂.H₂O (14 mL, 0.31 mol) was added and the solution was stirred at 70° C. for 3 hrs. After evaporated under reduced pressure (oil pump at 65° C.), the residue was added with water (500 mL), slurried and filtered. The cake was once more slurried with water (300 mL), filtered and dried to give the product as a yellow solid. MS: M/e 260 (M+1)⁺

### Step B: 2-(furan-2-yl)-7H-pyrazolo[4,3-e][1,2,4]triazolo[1,5-c]pyrimidin-5-amine

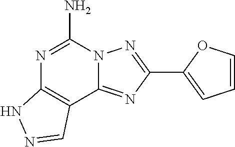 [SMILES: Nc1nc2[nH]ncc2c2nc(-c3ccco3)nn12]

A solution of N′-(6-amino-1H-pyrazolo[3,4-d]pyrimidin-4-yl)furan-2-carbohydrazide (17 g, 65 mmol) in BSA (136 mL) and HMDS (160 mL) was heated at 110° C. overnight. The solution was concentrated at 60° C. under reduced pressure. The residue was added with water (200 mL) and slurried for 1 hr. The solid was filtered, washed with water and dried to obtain the desired product as a brown solid (13.1 g, 83%). MS: M/e 242 (M+1)⁺

### Step C: ethyl 2-(5-amino-2-(furan-2-yl)-7H-pyrazolo[4,3-e][1,2,4]triazolo[1,5-c]pyrimidin-7-yl)propanoate

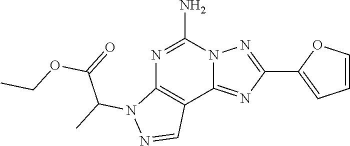 [SMILES: CCOC(=O)C(C)n1ncc2c1nc(N)n1nc(-c3ccco3)nc21]

To a stirred solution of 2-(furan-2-yl)-7H-pyrazolo[4,3-e][1,2,4]triazolo[1,5-c]pyrimidin-5-amine (500 mg, 2.07 mmol) in DMF (20 mL) and K₂CO₃ (344 mg, 2.49 mmol) was added ethyl 2-chloropropanoate (339 mg, 2.49 mmol) at RT. The mixture was stirred at rt overnight. The mixture was diluted with water (20 mL) and extracted with EA (20 mL×2). The residue (a mixture of N1 and N2 position compounds) was used into next step directly. MS: M/e 342 (M+1)⁺.

### Step D: 2-(5-amino-2-(furan-2-yl)-7H-pyrazolo[4,3-e][1,2,4]triazolo[1,5-c]pyrimidin-7-yl)propanoic Acid

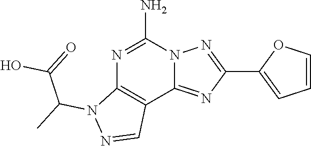 [SMILES: CC(C(=O)O)n1ncc2c1nc(N)n1nc(-c3ccco3)nc21]

To a stirred solution of the product of Step C (707 mg, crude, 2.07 mmol) in MeOH (12 mL) was added aq.NaOH (2.0 M, 4 mL) at RT. After the addition, the mixture was stirred at 60° C. for 3 hours. The reaction was concentrated under reduced pressure. The residue was dissolved into water (20 mL) and neutralized by HCl (2M) to pH=3˜4. The water phase was concentrated under reduced pressure. The residue (a mixture of N1 and N2 position compounds, contained NaCl) was used into next step directly. MS: M/e 314 (M+1)⁺.

### Step E: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)propan-1-one

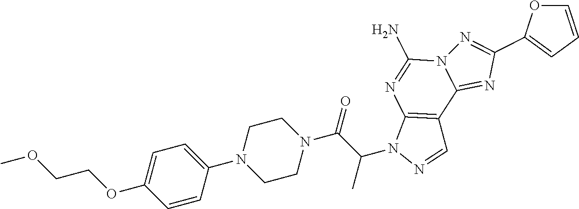 [SMILES: COCCOc1ccc(N2CCN(C(=O)C(C)n3ncc4c3nc(N)n3nc(-c5ccco5)nc43)CC2)cc1]

A mixture of the product of step D (200 mg, 0.64 mmol), 1-(4-(2-methoxyethoxy)phenyl)piperazine (190 mg, 0.7 mmol), HATU (267 mg, 0.7 mmol) and Et₃N (322 mg, 3.19 mmol) in DMF (10 mL) was stirred at RT overnight. The reaction mixture was poured into H₂O (20 mL) and extracted with EA (15 mL×3). The combined organic layers were washed with brine, dried over Na₂SO₄ and concentrated under reduced pressure. The residue was purified by prep-HPLC to afford the title compound. ¹H NMR (400 MHz, DMSO-d6) δ 8.20 (s, 1H), 8.18-8.13 (m, 2H), 7.99-7.90 (m, 1H), 7.26-7.21 (m, 1H), 6.96 (d, J=8.0 Hz, 2H), 6.85 (d, J=8.0 Hz, 2H), 6.77-6.71 (m, 1H), 5.81-5.68 (m, 1H), 4.04-3.96 (m, 2H), 3.60-3.53 (m, 4H), 3.52-3.37 (m, 2H), 3.28 (s, 3H), 3.20-2.93 (m, 4H), 1.61 (d, J=8.0 Hz, 3H) ppm. MS: M/e 532 (M+1)⁺.

Compound A1 was separated into two enantiomeric stereoisomers (Compound A1a, earlier peak, and Compound A1b, later peak) by chiral prep-HPLC. The chiral separation conditions are shown below.

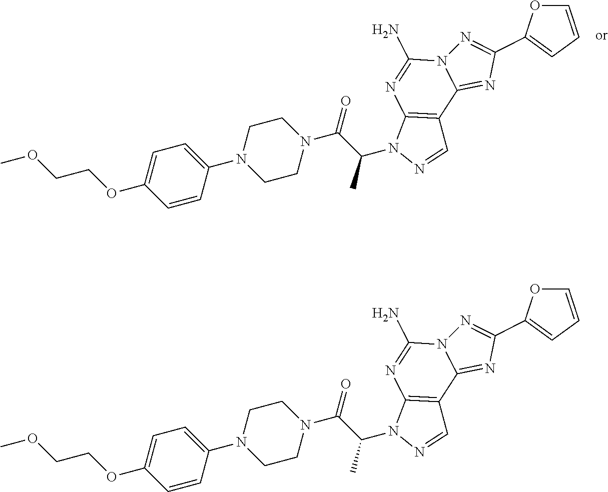 [SMILES: COCCOc1ccc(N2CCN(C(=O)[C@H](C)n3ncc4c3nc(N)n3nc(-c5ccco5)nc43)CC2)cc1 ; COCCOc1ccc(N2CCN(C(=O)[C@@H](C)n3ncc4c3nc(N)n3nc(-c5ccco5)nc43)CC2)cc1]

| Column | CHIRALPAK SA |
|---|---|
| Column size | 2 cm × 25 cm, 5 um |
| Injection | 1 mL |
| Mobile phase | CO₂:IPA(2 mM NH₃—MeOH) = 65:35 |
| Flow rate | 55 g/min |
| Wavelength | UV 220 nm |
| Temperature | 25° C. |
| Sample solution | 18.8 mg/ml in EtOH:DCM = 3:1 |
| Prep-SFC equipment | Prep-SFC-80 |

### Compound A2: 2-(5-amino-2-(furan-2-yl)-7H-pyrazolo[4,3-e][1,2,4]Triazolo[1,5-c]pyrimidin-7-yl)-1-(4-(4-(2-methoxyethoxy)phenyl)piperazin-1-yl)-3-methylbutan-1-one

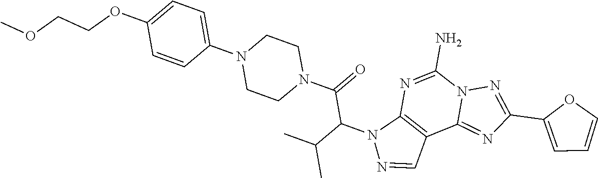 [SMILES: COCCOc1ccc(N2CCN(C(=O)C(C(C)C)n3ncc4c3nc(N)n3nc(-c5ccco5)nc43)CC2)cc1]

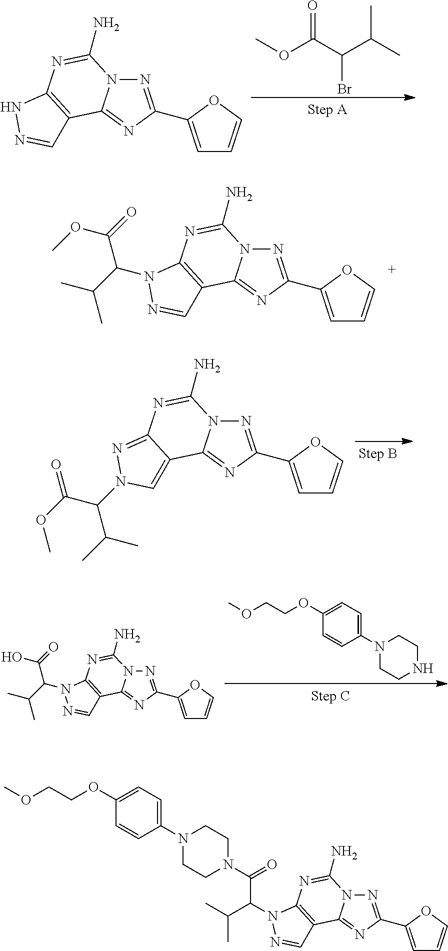

### Step A: methyl 2-(5-amino-2-(furan-2-yl)-7H-pyrazolo[4,3-e][1,2,4]triazolo[1,5-c]pyrimidin-7-yl)-3-methylbutanoate and methyl 2-(5-amino-2-(furan-2-yl)-8H-pyrazolo[4,3-e][1,2,4]triazolo[1,5-c]pyrimidin-8-yl)-3-methylbutanoate

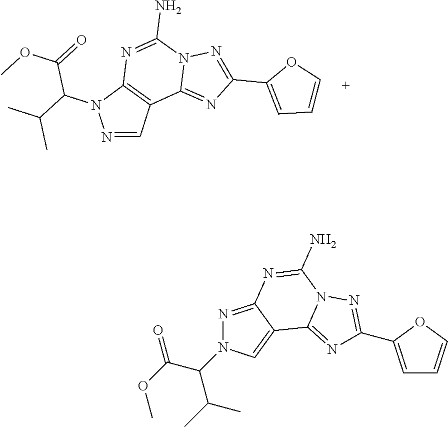 [SMILES: COC(=O)C(C(C)C)n1ncc2c1nc(N)n1nc(-c3ccco3)nc21 ; COC(=O)C(C(C)C)n1cc2c(nc(N)n3nc(-c4ccco4)nc23)n1]

A mixture of 2-(furan-2-yl)-7H-pyrazolo[4,3-e][1,2,4]triazolo[1,5-c]pyrimidin-5-amine (330 mg, 1.37 mmol), methyl 2-bromo-3-methylbutanoate (330 mg, 1.70 mmol) and K₂CO₃ (400 mg, 2.90 mmol) in DMF (7 mL) was heated at 50° C. for 16 hrs. The mixture was diluted with EA (30 mL) and the suspension was filtered. The filtrate was washed with brine (15 mL×3), dried over Na₂SO₄ and concentrated. The resulting residue was purified by prep-TLC to give methyl 2-(5-amino-2-(furan-2-yl)-7H-pyrazolo[4,3-e][1,2,4]triazolo[1,5-c]pyrimidin-7-yl)-3-methylbutanoate (70 mg) as a white solid, MS: M/e 356 (M+1)⁺, and methyl 2-(5-amino-2-(furan-2-yl)-8H-pyrazolo[4,3-e][1,2,4]triazolo[1,5-c]pyrimidin-8-yl)-3-methylbutanoate (170 mg) as a white solid, MS: M/e 356 (M+1)⁺.

### Step B: 2-(5-amino-2-(furan-2-yl)-7H-pyrazolo[4,3-e][1,2,4]triazolo[1,5-c]pyrimidin-7-yl)-3-methylbutanoic Acid

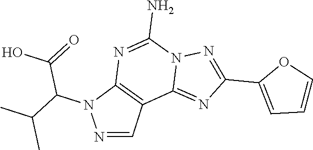 [SMILES: CC(C)C(C(=O)O)n1ncc2c1nc(N)n1nc(-c3ccco3)nc21]

To a stirred solution of the product from Step A (60 mg, 0.17 mmol) in MeOH (2 mL) was added aqueous solution of NaOH (2 M, 1 mL) at rt and the resulting mixture was stirred for 2 hrs. The mixture was neutralized by HCl (1 M) and concentrated to dryness. 5 mL of a mixed solvent (CH₂Cl₂/MeOH=3:1) was added and stirred for 10 min. The suspension was filtered and the filtrate was concentrated to give the title product (52 mg, 90%) as a white solid. MS: M/e 342 (M+1)⁺.

### Step C: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-3-methylbutan-1-one

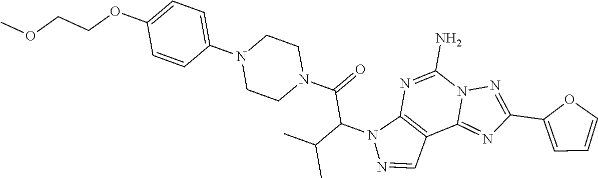 [SMILES: COCCOc1ccc(N2CCN(C(=O)C(C(C)C)n3ncc4c3nc(N)n3nc(-c5ccco5)nc43)CC2)cc1]

To a mixture of the product from step B (52 mg, 0.15 mmol), 1-(4-(2-methoxyethoxy)phenyl)piperazine (52 mg, 0.22 mmol), DIEA (100 mg, 0.78 mmol) in DMF (2 mL) was added HATU (70 mg, 0.18 mmol) at rt and the mixture was stirred at rt for 2 hrs. 20 mL of EA was added and the mixture was washed with brine (10 mL×3), dried over Na₂SO₄ and concentrated. The resulting residue was purified by prep-TLC (EA/MeOH=20:1) to give the title product (46.0 mg, yield: 55%). ¹H NMR (400 MHz, DMSO-d6) δ 8.40-8.10 (m, 3H), 7.95 (s, 1H), 7.23 (d, J=3.6 Hz, 1H), 6.82-6.71 (m, 5H), 5.20 (d, J=9.6 Hz, 1H), 4.00-3.92 (m, 2H), 3.80-3.71 (m, 1H), 3.69-3.62 (m, 2H), 3.61-3.56 (m, 2H), 3.53-3.45 (m, 1H), 3.27 (s, 3H), 3.06-2.97 (m, 1H), 2.97-2.90 (m, 1H), 2.84-2.69 (m, 2H), 2.32-2.23 (m, 1H), 1.04 (d, J=6.8 Hz, 3H), 0.60 (d, J=6.8 Hz, 3H). MS: M/e 560 (M+1)⁺.

### Compound A3: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-methylpropan-1-one

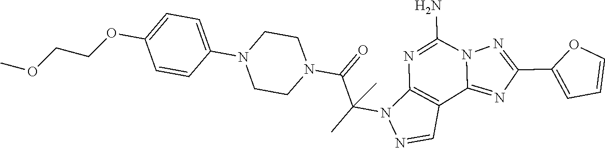 [SMILES: COCCOc1ccc(N2CCN(C(=O)C(C)(C)n3ncc4c3nc(N)n3nc(-c5ccco5)nc43)CC2)cc1]

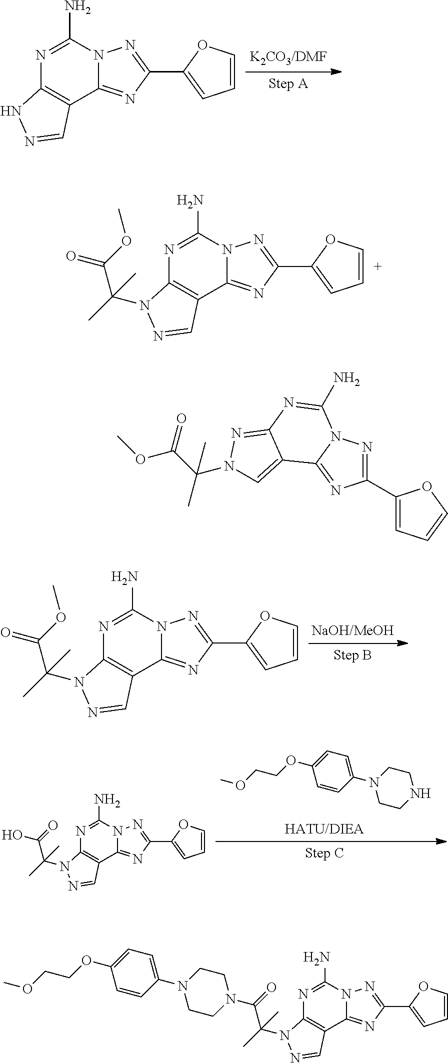

### Step A: methyl 2-(5-amino-2-(furan-2-yl)-7H-pyrazolo[4,3-e][1,2,4]triazolo[1,5-c]pyrimidin-7-yl)-2-methylpropanoate and methyl 2-(5-amino-2-(furan-2-yl)-8H-pyrazolo[4,3-e][1,2,4]triazolo[1,5-c]pyrimidin-8-yl)-2-methylpropanoate

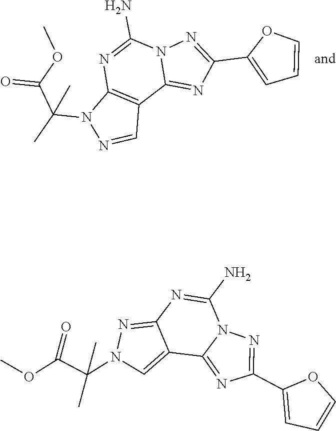 [SMILES: COC(=O)C(C)(C)n1ncc2c1nc(N)n1nc(-c3ccco3)nc21 ; COC(=O)C(C)(C)n1cc2c(nc(N)n3nc(-c4ccco4)nc23)n1]

A mixture of 2-(furan-2-yl)-7H-pyrazolo[4,3-e][1,2,4]triazolo[1,5-c]pyrimidin-5-amine (1.5 g, 6.2 mmol), methyl 2-bromo-2-methylpropanoate (1.3 g, 7.4 mmol) and K₂CO₃ (1.7 g, 12.4 mmol) in DMF (80 mL) was stirred at rt overnight. The solution was added with water (50 mL), extracted with ethyl aceate (50 mL) and washed with brine (50 mL). The organic layer was dried over Na₂SO₄, concentrated and purified by column chromatography (PE:EA=3:1 to 1:1) to get methyl 2-(5-amino-2-(furan-2-yl)-7H-pyrazolo[4,3-e][1,2,4]triazolo[1,5-c]pyrimidin-7-yl)-2-methylpropanoate (65 mg, 3%). ¹H NMR (400 MHz, DMSO-d6) δ 8.19 (s, 1H), 8.06 (br.s, 2H), 7.95 (d, J=4.0 Hz, 1H), 7.23 (d, J=4.0 Hz, 1H), 6.73 (t, J=4.0 Hz, 1H), 3.67 (s, 3H), 1.87 (s, 6H) ppm. MS: M/e 342 (M+1)⁺ and methyl 2-(5-amino-2-(furan-2-yl)-8H-pyrazolo[4,3-e][1,2,4]triazolo[1,5-c]pyrimidin-8-yl)-2-methylpropanoate (1.2 g, 57%). ¹H NMR (400 MHz, DMSO-d6) δ 8.87 (s, 1H), 7.95 (s, 1H), 7.67 (br.s, 1H), 7.18 (s, 1H), 6.74 (t, J=4.0 Hz, 1H), 3.64 (s, 3H), 1.87 (s, 6H) ppm. MS: M/e 342 (M+1)⁺

### Step B: 2-(5-amino-2-(furan-2-yl)-7H-pyrazolo[4,3-e][1,2,4]triazolo[1,5-c]pyrimidin-7-yl)-2-methylpropanoic Acid

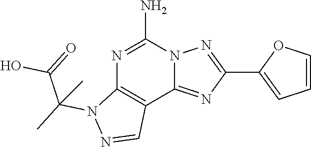 [SMILES: CC(C)(C(=O)O)n1ncc2c1nc(N)n1nc(-c3ccco3)nc21]

NaOH solution (30 mg, in 2 mL of water) was added to a solution of methyl 2-(5-amino-2-(furan-2-yl)-7H-pyrazolo[4,3-e][1,2,4]triazolo[1,5-c]pyrimidin-7-yl)-2-methylpropanoate (65 mg, 0.19 mmol) in methanol (5 mL). The reaction mixture was stirred at rt overnight. The solution was concentrated, added with water (5 mL) and acidified with 1N HCl solution to pH=5. The precipitated solid was filtered and dried to get the desired product as a white solid (53 mg, 85%). MS: M/e 328 (M+1)⁺

### Step C: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-methylpropan-1-one

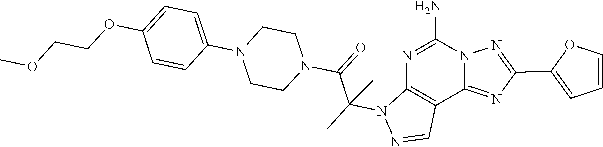 [SMILES: COCCOc1ccc(N2CCN(C(=O)C(C)(C)n3ncc4c3nc(N)n3nc(-c5ccco5)nc43)CC2)cc1]

A mixture of 2-(5-amino-2-(furan-2-yl)-7H-pyrazolo[4,3-e][1,2,4]triazolo[1,5-c]pyrimidin-7-yl)-2-methylpropanoic acid (53 mg, 0.16 mmol), 1-(4-(2-methoxyethoxy)phenyl)piperazine (38 mg, 0.16 mmol), HATU (73 mg, 0.19 mmol) and DIEA (42 mg, 0.32 mmol) in DMF (5 mL) was stirred at rt for 2 hrs. The solution was added with water (5 mL), extracted with ethyl acetate (10 mL) and washed with brine (10 mL). The organic layer was dried, concentrated and purified by column chromatography (PE:EA=2:1 to EA) to get the desired product (34 mg, 39%). ¹H NMR (400 MHz, DMSO-d6) δ 8.29 (s, 1H), 8.07 (br.s, 2H), 7.94 (s, 1H), 7.21 (d, J=4.0 Hz, 1H), 6.73-6.66 (m, 5H), 3.93 (d, J=4.0 Hz, 2H), 3.63 (br.s, 2H), 3.56 (d, J=4.0 Hz, 2H), 3.31 (s, 3H), 2.89 (br.s, 4H), 2.42 (br.s, 2H), 1.83 (s, 6H) ppm. MS: M/e 546 (M+1)⁺

### Compound A4: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)butan-1-one

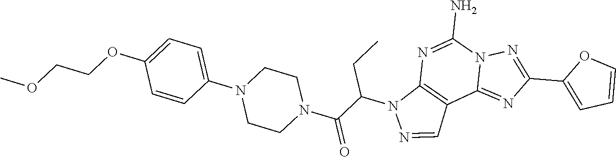 [SMILES: CCC(C(=O)N1CCN(c2ccc(OCCOC)cc2)CC1)n1ncc2c1nc(N)n1nc(-c3ccco3)nc21]

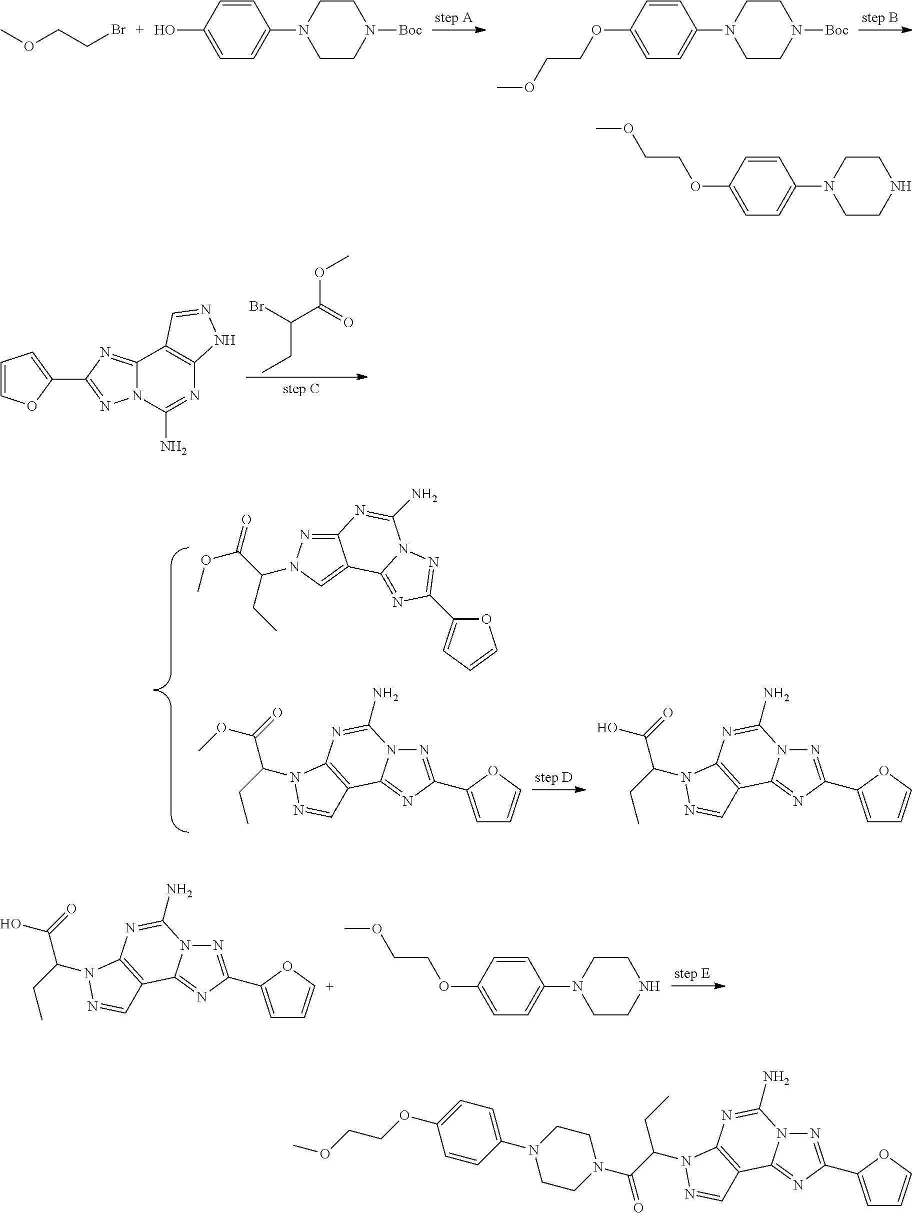

### Step A: tert-butyl 4-(4-(2-methoxyethoxy)phenyl)piperazine-1-carboxylate

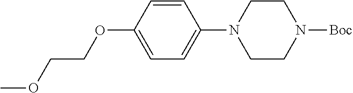 [SMILES: COCCOc1ccc(N2CCN(C(=O)OC(C)(C)C)CC2)cc1]

To a stirred solution of tert-butyl 4-(4-hydroxyphenyl)piperazine-1-carboxylate (10.0 g, 36 mmol) in CH₃CN (250 mL) was added NaH (3.0 g, 125 mmol), the reaction mixture was stirred for 30 min at 50° C. Then 1-bromo-2-methoxyethane (6.0 g, 43.5 mmol) was added into the reaction. After the addition, the reaction mixture was stirred overnight at 60° C. Most of solvent was removed and this residue was dissolved with H₂O (50 mL), then acidified to pH=6˜7 with aq.HCl and extracted with EtOAc (200 mL×3). The combined organic layers were washed with brine, dried over Na₂SO₄, concentrated to give tert-butyl 4-(4-(2-methoxyethoxy)phenyl)piperazine-1-carboxylate (16 g, crude) as yellow oil. MS: M/e 337 (M+1)⁺.

### Step B: 1-(4-(2-methoxyethoxy)phenyl)piperazine

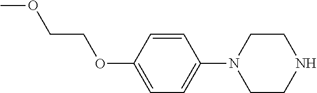 [SMILES: COCCOc1ccc(N2CCNCC2)cc1]

To a stirred mixture of tert-butyl 4-(4-(2-methoxyethoxy)phenyl)piperazine-1-carboxylate (16.0 g, crude) in DCM (100 mL) was added TFA (100 mL). After the addition, the reaction mixture was stirred for 3 hours at rt Most of solvent was removed and this residue was dissolved in H₂O (50 mL), extracted with EtOAc (50 mL), then adjusted to pH=9˜10 with aq. NaOH and extracted with EtOAc (200 mL×3). The combined organic layers were washed with brine, dried over Na₂SO₄, concentrated to give 1-(4-(2-methoxyethoxy)phenyl)piperazine (10 g, 88.8%) as a gray solid. MS: M/e 237 (M+1)⁺

### Step C: methyl 2-(5-amino-2-(furan-2-yl)-7H-pyrazolo[4,3-e][1,2,4]triazolo[1,5-c]pyrimidin-7-yl)butanoate and methyl 2-(5-amino-2-(furan-2-yl)-8H-pyrazolo[4,3-e][1,2,4]triazolo[1,5-c]pyrimidin-8-yl)butanoate

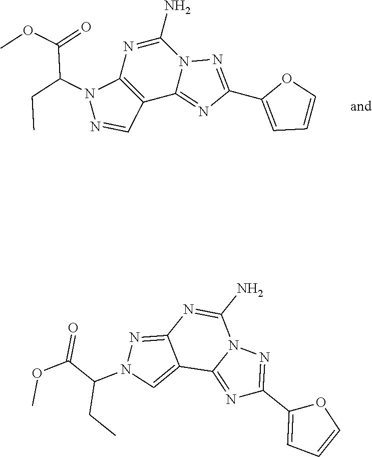 [SMILES: CCC(C(=O)OC)n1ncc2c1nc(N)n1nc(-c3ccco3)nc21 ; CCC(C(=O)OC)n1cc2c(nc(N)n3nc(-c4ccco4)nc23)n1]

To a stirred solution of 2-(furan-2-yl)-7H-pyrazolo[4,3-e][1,2,4]triazolo[1,5-c]pyrimidin-5-amine (300 mg, 1.25 mmol) in DMF (30 mL) was added K₂CO₃ (345 mg, 2.5 mmol) and methyl 2-bromo-butanoate (270 mg, 1.5 mmol). After the addition, the reaction mixture was stirred overnight. The reaction mixture was poured into H₂O (50 mL) and extracted with EtOAc (50 mL×3). The combined organic layers were washed with brine, dried over Na₂SO₄, concentrated and purified by column chromatography (petroleum ether/EtOAc=3:1˜1:1) to give methyl 2-(5-amino-2-(furan-2-yl)-7H-pyrazolo[4,3-e][1,2,4]triazolo[1,5-c]pyrimidin-7-yl)butanoate (120 mg, 56.1%) and methyl 2-(5-amino-2-(furan-2-yl)-8H-pyrazolo[4,3-e][1,2,4]triazolo[1,5-c]pyrimidin-8-yl)butanoate (100 mg, 46.8%) as white solids. MS: M/e 342 (M+1)⁺.

### Step D: 2-(5-amino-2-(furan-2-yl)-7H-pyrazolo[4,3-e][1,2,4]triazolo[1,5-c]pyrimidin-7-yl)butanoic acid

To a stirred mixture of methyl 2-(5-amino-2-(furan-2-yl)-7H-pyrazolo[4,3-e][1,2,4]triazolo[1,5-c]pyrimidin-7-yl)butanoate (120 mg, 0.35 mmol) in MeOH/H₂O (3 mL/1 mL) was added aq.NaOH (2.0 M, 1 mL). After the addition, the reaction mixture was stirred for 5 hours at RT. Most of the solvent was removed to give the aqueous layer, then acidified to pH=3˜4 with aq.HCl and filtered, the cake was collected, dried to give the target compound (100 mg, 87.1%) as a white solid. MS: M/e 328 (M+1)⁺.

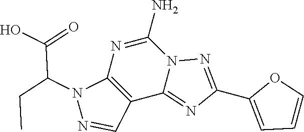 [SMILES: CCC(C(=O)O)n1ncc2c1nc(N)n1nc(-c3ccco3)nc21]

### Step E: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)butan-1-one

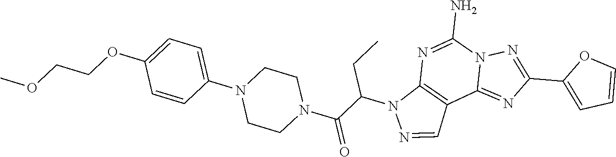 [SMILES: CCC(C(=O)N1CCN(c2ccc(OCCOC)cc2)CC1)n1ncc2c1nc(N)n1nc(-c3ccco3)nc21]

A mixture of the product of step D (80 mg, 0.25 mmol), 1-(4-(2-methoxyethoxy)phenyl)piperazine (80 mg, 0.34 mmol), HATU (100 mg, 0.25 mmol) and TEA (80 mg, 0.79 mmol) in CH₃CN (20 mL) was stirred overnight. The reaction mixture was poured into H₂O (20 mL) and extracted with EtOAc (40 mL×3). The combined organic layers were washed with brine, dried over Na₂SO₄, concentrated and purified by column chromatography (petroleum ether/EtOAc=1:1˜100% EtOAc) to give the target compound (30 mg, 22.0%). ¹H NMR (400 MHz, DMSO-d6) δ 8.20 (s, 1H), 8.18 (br, 2H), 7.95 (s, 1H), 7.24 (d, J=2.7 Hz, 1H), 6.83-6.76 (m, 4H), 6.74 (d, J=1.5 Hz, 1H), 5.56-5.48 (m, 1H), 3.98 (d, J=3.7 Hz, 2H), 3.69 (s, 1H), 3.63-3.46 (m, 5H), 3.27 (s, 3H), 3.06-2.91 (m, 2H), 2.80 (s, 1H), 2.44 (s, 1H), 2.21-2.09 (m, 2H), 0.79 (t, J=7.1 Hz, 3H) ppm. MS: M/e 546 (M+1)⁺.

### Compound A5: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)pentan-1-one

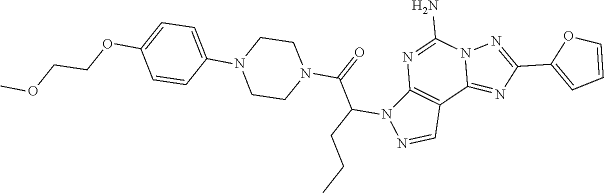 [SMILES: CCCC(C(=O)N1CCN(c2ccc(OCCOC)cc2)CC1)n1ncc2c1nc(N)n1nc(-c3ccco3)nc21]

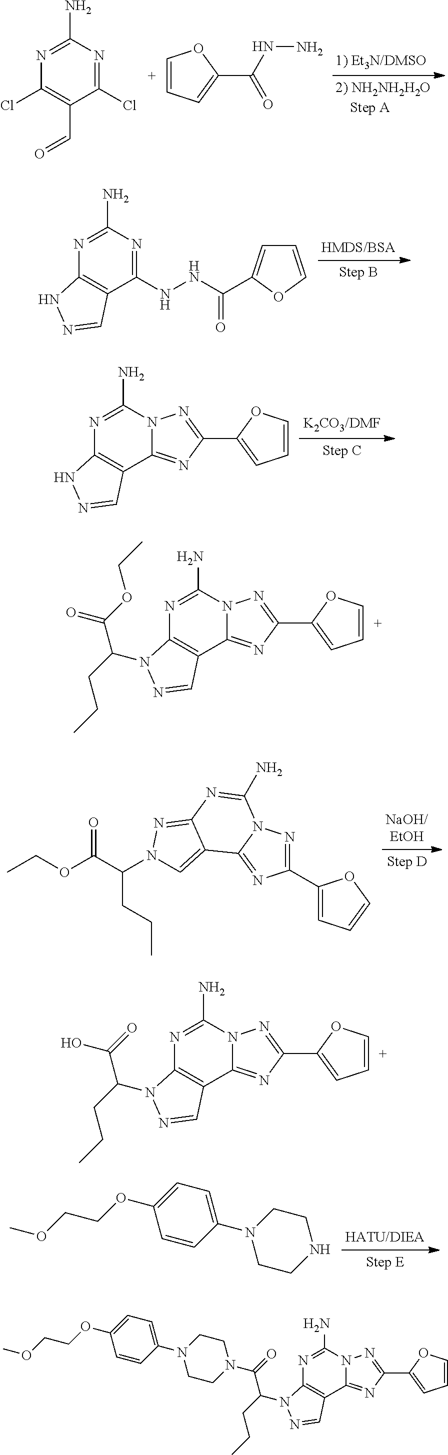

### Step A: N′-(6-amino-1H-pyrazolo[3,4-d]pyrimidin-4-yl)furan-2-carbohydrazide

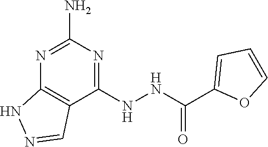 [SMILES: Nc1nc(NNC(=O)c2ccco2)c2cn[nH]c2n1]

To a stirred solution of 2-amino-4,6-dichloropyrimidine-5-carbaldehyde (50 g, 0.26 mol) and Et₃N (28.9 g, 0.28 mol) in DMSO (600 mL) was added with furan-2-carbohydrazide (32.8 g, 0.26 mol) in portionwise. Then the reaction mixture was stirred at rt overnight. LCMS showed the starting materials were converted into the intermediate. NH₂NH₂.H₂O (14 mL, 0.31 mol) was added and the solution was stirred at 70° C. for 3 hrs. After evaporation under reduced pressure (oil pump at 65° C.), the residue was added with water (500 mL), slurried and filtered. The cake was once more slurried with water (300 mL), filtered and dried to give the product as a yellow solid. MS: M/e 260 (M+1)⁺

### Step B: 2-(furan-2-yl)-7H-pyrazolo[4,3-e][1,2,4]triazolo[1,5-c]pyrimidin-5-amine

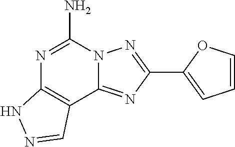 [SMILES: Nc1nc2[nH]ncc2c2nc(-c3ccco3)nn12]

A solution of N′-(6-amino-1H-pyrazolo[3,4-d]pyrimidin-4-yl)furan-2-carbohydrazide (17 g, 65 mmol) in BSA (136 mL) and HMDS (160 mL) was heated at 110° C. overnight. The solution was concentrated at 60° C. under reduced pressure. The residue was added with water (200 mL) and slurried for 1 hr. The solid was filtered, washed with water and dried to obtain the desired product as a brown solid (13.1 g, 83%). MS: M/e 242 (M+1)⁺

### Step C: ethyl 2-(5-amino-2-(furan-2-yl)-7H-pyrazolo[4,3-e][1,2,4]triazolo[1,5-c]pyrimidin-7-yl)pentanoate and ethyl 2-(5-amino-2-(furan-2-yl)-8H-pyrazolo[4,3-e][1,2,4]triazolo[1,5-c]pyrimidin-8-yl)pentanoate

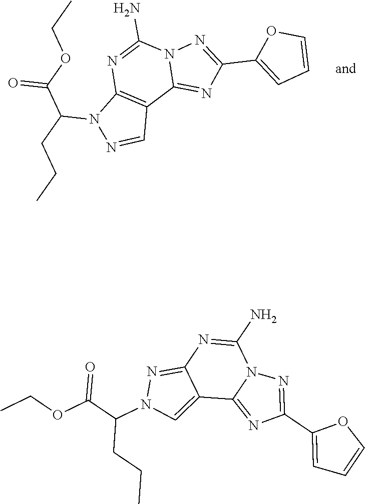 [SMILES: CCCC(C(=O)OCC)n1ncc2c1nc(N)n1nc(-c3ccco3)nc21 ; CCCC(C(=O)OCC)n1cc2c(nc(N)n3nc(-c4ccco4)nc23)n1]

A mixture of 2-(furan-2-yl)-7H-pyrazolo[4,3-e][1,2,4]triazolo[1,5-c]pyrimidin-5-amine (2 g, 8.3 mmol), ethyl 2-bromopentanoate (2.1 g, 9.9 mmol) and K₂CO₃ (2.3 g, 16.6 mmol) in DMF (50 mL) was stirred at rt overnight. The solution was added with water (30 mL), extracted with ethyl acetate (50 mL) and washed with brine (50 mL). The organic layer was dried over Na₂SO₄, concentrated and purified by column chromatography (PE:EA=4:1 to 2:1) to get ethyl 2-(5-amino-2-(furan-2-yl)-7H-pyrazolo[4,3-e][1,2,4]triazolo[1,5-c]pyrimidin-7-yl)pentanoate (600 mg, 20%) and ethyl 2-(5-amino-2-(furan-2-yl)-8H-pyrazolo[4,3-e][1,2,4]triazolo[1,5-c]pyrimidin-8-yl)pentanoate (810 mg, 25%). MS: M/e 370 (M+1)⁺

### Step D: 2-(5-amino-2-(furan-2-yl)-7H-pyrazolo[4,3-e][1,2,4]triazolo[1,5-c]pyrimidin-7-yl)pentanoic Acid

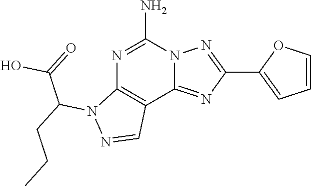 [SMILES: CCCC(C(=O)O)n1ncc2c1nc(N)n1nc(-c3ccco3)nc21]

NaOH solution (324 mg, in 2 mL of water) was added to a solution of ethyl 2-(5-amino-2-(furan-2-yl)-7H-pyrazolo[4,3-e][1,2,4]triazolo[1,5-c]pyrimidin-7-yl)pentanoate (600 mg, 1.6 mmol) in ethanol (10 mL). The reaction mixture was stirred at rt for 3 hrs. The solution was concentrated, added with water (10 mL) and acidified with 1N HCl solution to pH=5. The precipitated solid was filtered and dried to get the desired product as a white solid (475 mg, 86%). MS: M/e 342 (M+1)⁺

### Step E: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)pentan-1-one

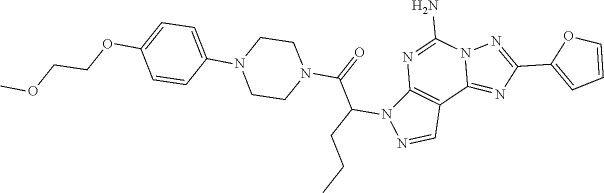 [SMILES: CCCC(C(=O)N1CCN(c2ccc(OCCOC)cc2)CC1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)pentanoic acid (350 mg, 1 mmol), 1-(4-(2-methoxyethoxy)phenyl)piperazine (241 mg, 1 mmol), HATU (456 mg, 1.2 mmol) and DIEA (258 mg, 2 mmol) in DMF (20 mL) was stirred at rt for 2 hrs. The solution was added with water (15 mL), extracted with ethyl acetate (20 mL) and washed with brine (20 mL). The organic layer was dried, concentrated and purified by column chromatography (PE:EA=1:1 to EA) to get the desired product (330 mg, 57%). ¹H NMR (400 MHz, DMSO-d6) δ 8.20 (s, 1H), 8.17 (br.s, 2H), 7.95 (s, 1H), 7.23 (d, J=4.0 Hz, 1H), 6.79-6.73 (m, 5H), 5.58 (dd, J=8.0 Hz, 4.0 Hz 1H), 3.98-3.95 (m, 2H), 3.69-3.52 (m, 6H), 3.27 (s, 3H), 3.00-2.79 (m, 3H), 2.16-2.02 (m, 3H), 1.23-1.09 (m, 2H), 0.87 (t, J=8.0 Hz, 3H) ppm. MS: M/e 560 (M+1)⁺

Compound A5 was separated into two enantiomeric stereoisomers (Compound A5a, earlier peak, and Compound A5b, later peak) by chiral prep-HPLC. The chiral separation conditions are shown below.

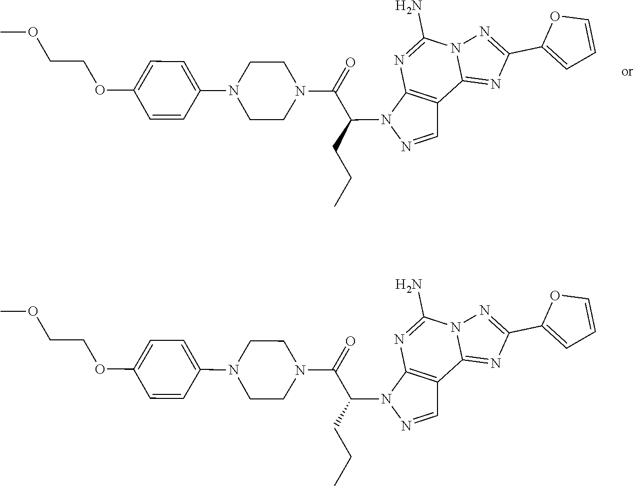 [SMILES: CCC[C@@H](C(=O)N1CCN(c2ccc(OCCOC)cc2)CC1)n1ncc2c1nc(N)n1nc(-c3ccco3)nc21 ; CCC[C@H](C(=O)N1CCN(c2ccc(OCCOC)cc2)CC1)n1ncc2c1nc(N)n1nc(-c3ccco3)nc21]

| Column | CHIRALPAK IC |
|---|---|
| Column size | 2 cm × 25 cm, 5 um |
| Injection | 0.8 mL |
| Mobile phase | (Hex:DCM = 1:1):EtOH = 50:50 |
| Flow rate | 18 ml/min |
| Wavelength | UV 220 nm |
| Temperature | 25° C. |
| Sample solution | 27.8 mg/ml in EtOH:DCM = 1:1 |
| Prep-HPLC equipment | Prep-Gilson-HPLC |

### Compound A6: 2-(5-amino-2-(furan-2-yl)-7H-pyrazolo[4,3-e][1,2,4]triazolo[1,5-c]pyrimidin-7-yl)-1-(4-(4-(2-(2-methoxyethoxy)ethoxy)phenyl)piperazin-1-yl)pentan-1-one

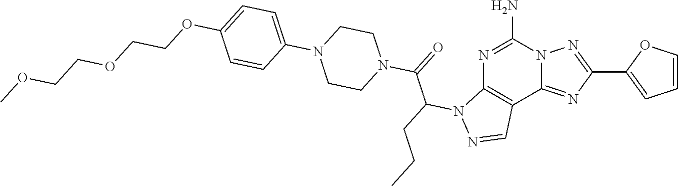 [SMILES: CCCC(C(=O)N1CCN(c2ccc(OCCOCCOC)cc2)CC1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)pentanoic acid (375 mg, 1.1 mmol), 1-(4-(2-(2-methoxyethoxy)ethoxy)phenyl)piperazine (338 mg, 1.2 mmol), HATU (502 mg, 1.3 mmol) and DIEA (284 mg, 2.2 mmol) in DMF (10 mL) was stirred at rt for 2 hrs. The solution was added with water (10 mL), extracted with ethyl acetate (10 mL) and washed with brine (10 mL). The organic layer was dried, concentrated and purified by column chromatography (PE:EA=1:1 to EA) to get the desired product (480 mg, 73%). ¹H NMR (400 MHz, DMSO-d6) δ 8.20 (s, 1H), 8.16 (br.s, 2H), 7.95 (s, 1H), 7.23 (s, 1H), 6.82-6.73 (m, 5H), 5.60-5.56 (m, 1H), 4.04-3.96 (m, 2H), 3.60-3.42 (m, 10H), 3.23 (s, 3H), 3.05-2.94 (m, 2H), 2.67 (br.s, 1H), 2.33 (br.s, 1H), 2.06-1.99 (m, 2H), 1.24-1.10 (m, 1H), 0.92-0.85 (m, 3H) ppm. MS: M/e 604 (M+1)⁺

Compound A6 was separated into two enantiomeric stereoisomers (Compound A6a, earlier peak, and Compound A6b, later peak) by chiral prep-HPLC. The chiral separation conditions are shown below.

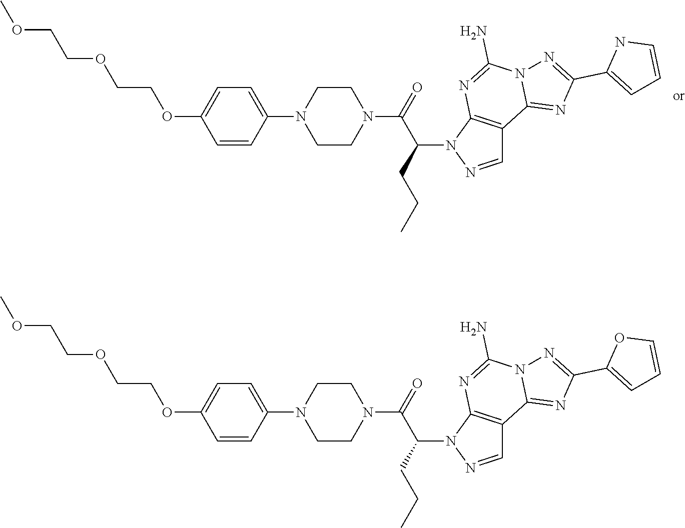 [SMILES: CCC[C@@H](C(=O)N1CCN(c2ccc(OCCOCCOC)cc2)CC1)n1ncc2c1nc(N)n1nc(-c3ccc[nH]3)nc21 ; CCC[C@H](C(=O)N1CCN(c2ccc(OCCOCCOC)cc2)CC1)n1ncc2c1nc(N)n1nc(-c3ccco3)nc21]

| Column | CHIRALPAK Cellulose-IC |
|---|---|
| Column size | 2 cm × 25 cm, 5 um |
| Injection | 0.8 mL |
| Mobile phase | (Hex:DCM = 50:50):EtOH = 50:50 |
| Flow rate | 15 ml/min |
| Wavelength | UV 220 nm |
| Temperature | 25° C. |
| Sample solution | 48.67 mg/ml in DCM:EtOH = 1:1 |
| Prep-HPLC equipment | Prep-Gilson-HPLC |

### Compound A7: 2-(5-amino-2-(furan-2-yl)-7H-pyrazolo[4,3-e][1,2,4]triazolo[1,5-c]pyrimidin-7-yl)-1-(4-(4-(2-aminoethoxy)phenyl)piperazin-1-yl)propan-1-one

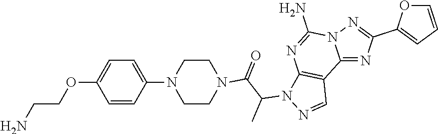 [SMILES: CC(C(=O)N1CCN(c2ccc(OCCN)cc2)CC1)n1ncc2c1nc(N)n1nc(-c3ccco3)nc21]

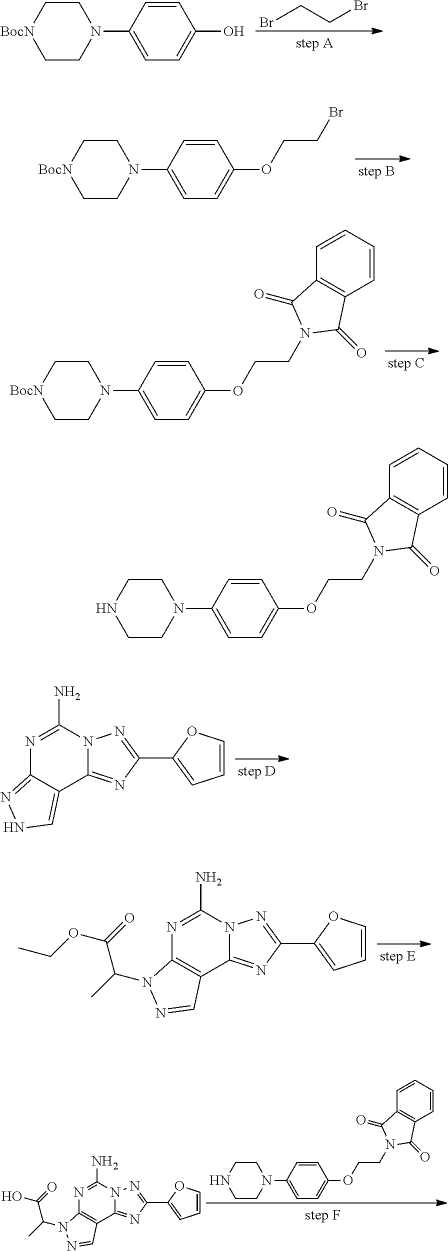

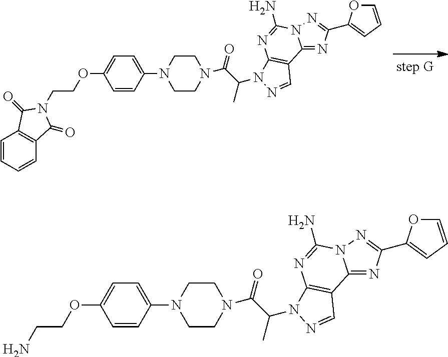 [SMILES: CC(C(=O)N1CCN(c2ccc(OCCN3C(=O)c4ccccc4C3=O)cc2)CC1)n1ncc2c1nc(N)n1nc(-c3ccco3)nc21 ; CC(C(=O)N1CCN(c2ccc(OCCN)cc2)CC1)n1ncc2c1nc(N)n1nc(-c3ccco3)nc21]

### Step A: tert-butyl 4-(4-(2-bromoethoxy)phenyl)piperazine-1-carboxylate

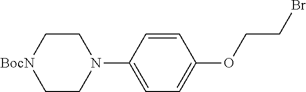 [SMILES: CC(C)(C)OC(=O)N1CCN(c2ccc(OCCBr)cc2)CC1]

The mixture of tert-butyl 4-(4-hydroxyphenyl)piperazine-1-carboxylate, 1,2-dibromoethane and K₂CO₃ in Acetone was stirred at rt overnight. The mixture was diluted with water (300 mL) and extracted with EA (300 mL×3). The combined organic phase was washed with brine, dried over Na₂SO₄ and concentrated under reduced pressure. The residue was purified by silica gel chromatography (eluted with EA:PE 1:5) to afford the title compound (3.2 g, yield: 32.3%) as yellow oil. MS: M/e 385 (M+1)⁺.

### Step B: tert-butyl 4-(4-(2-(1,3-dioxoisoindolin-2-yl)ethoxy)phenyl)piperazine-1-carboxylate

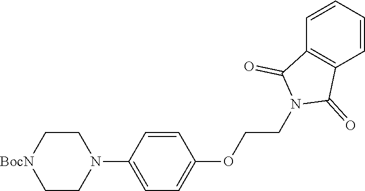 [SMILES: CC(C)(C)OC(=O)N1CCN(c2ccc(OCCN3C(=O)c4ccccc4C3=O)cc2)CC1]

A mixture of the product of step A (6.2 g, 16.1 mmol) and isoindoline-1,3-dione (2.98 g, 16.1 mmol) in DMF (100 mL) was stirred at 80° C. for 2 hours. The reaction was quenched by water (200 mL) and the precipitate was formed from the system. The mixture was filtered and the solid was collected. The yellow solid was dried in air and used into next step directly. ¹H NMR (400 MHz, DMSO-d6) δ 7.97-7.68 (m, 4H), 6.94-6.70 (m, 4H), 4.30-4.06 (m, 2H), 3.99-3.84 (m, 2H), 3.46-3.37 (m, 4H), 2.97-2.84 (m, 4H), 1.41 (s, 9H) ppm. MS: M/e 452 (M+1)⁺.

### Step C: 2-(2-(4-(piperazin-1-yl)phenoxy)ethyl)isoindoline-1,3-dione

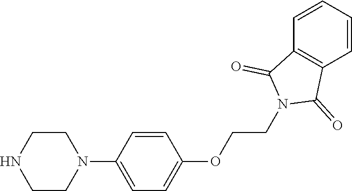 [SMILES: O=C1c2ccccc2C(=O)N1CCOc1ccc(N2CCNCC2)cc1]

A mixture of the product of step B (6.2 g, 16.1 mmol) in HCl/1,4-dioxane (100 mL) was stirred at RT for 3 hours. The solid was formed from the system. The mixture was filtered and solid was washed with EA (50 mL). The white solid (3.7 g, yield: 93.7%) was dried in air and used into next step directly. MS: M/e 352 (M+1)⁺.

### Step D: ethyl 2-(5-amino-2-(furan-2-yl)-7H-pyrazolo[4,3-e][1,2,4]triazolo[1,5-c]pyrimidin-7-yl)propanoate

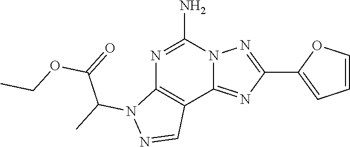 [SMILES: CCOC(=O)C(C)n1ncc2c1nc(N)n1nc(-c3ccco3)nc21]

To a stirred solution of 2-(furan-2-yl)-7H-pyrazolo[4,3-e][1,2,4]triazolo[1,5-c]pyrimidin-5-amine (3 g, 12.4 mmol) in DMF (50 mL) was added K₂CO₃ (2.06 mg, 14.9 mmol) and ethyl 2-chloropropanoate (2.03 mg, 14.9 mmol). The reaction was quenched with water (50 mL) and extracted with EA (50 mL×3). The combined organic phase was washed with brine, dried over Na₂SO₄ and concentrated under reduced pressure. The residue was purified by silica gel chromatography (petroleum ether/EA=1:1) to afford the title compound (1 g, 47.2%) as white solids. MS: M/e 342 (M+1)⁺.

### Step E: 2-(5-amino-2-(furan-2-yl)-7H-pyrazolo[4,3-e][1,2,4]triazolo[1,5-c]pyrimidin-7-yl)propanoic Acid

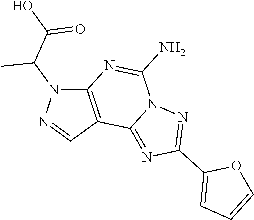 [SMILES: CC(C(=O)O)n1ncc2c1nc(N)n1nc(-c3ccco3)nc21]

To a stirred solution of the product of Step D (1 g, 2.9 mmol) in MeOH (24 mL) was added aq.NaOH (2.0 M, 8 mL) at rt. The mixture was stirred at rt overnight. The solvents were removed and the residue was dissolved into water (20 mL). The mixture was acidified to pH=3˜4 with aq.HCl (2 M). The solid was precipitated from the system. The mixture was filtered and the solid was collected. The white solid (620 mg, 93.8%) was dried in air and used into next step directly. MS: M/e 314 (M+1)⁺.

### Step F: 2-(2-(4-(4-(2-(5-amino-2-(furan-2-yl)-7H-pyrazolo[4,3-e][1,2,4]triazolo[1,5-c]pyrimidin-7-yl)propanoyl)piperazin-1-yl)phenoxy)ethyl)isoindoline-1,3-dione

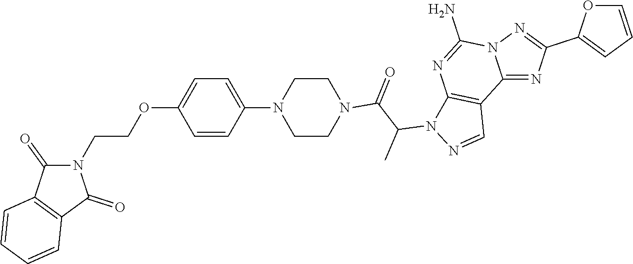 [SMILES: CC(C(=O)N1CCN(c2ccc(OCCN3C(=O)c4ccccc4C3=O)cc2)CC1)n1ncc2c1nc(N)n1nc(-c3ccco3)nc21]

A mixture of the product of Step C (564 mg, 1.6 mmol), 2-(5-amino-2-(furan-2-yl)-7H-pyrazolo[4,3-e][1,2,4]triazolo[1,5-c]pyrimidin-7-yl)propanoic acid (the product of Step E, 500 mg, 1.6 mmol), HATU (668 mg, 1.76 mmol) and Et₃N (1 mL, excess) in DMF (20 mL) was stirred at RT overnight. The reaction mixture was poured into water (40 mL) and the solid was precipitated from the system. The solid was filtered and dried in air. The yellow solid (510 mg, yield: 49.1%) was used into next step without further purification. MS: M/e 647 (M+1)⁺.

### Step G: 2-(5-amino-2-(furan-2-yl)-7H-pyrazolo[4,3-e][1,2,4]triazolo[1,5-c]pyrimidin-7-yl)-1-(4-(4-(2-aminoethoxy)phenyl)piperazin-1-yl)propan-1-one

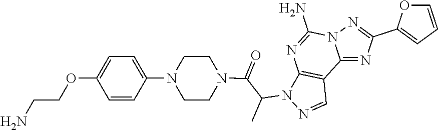 [SMILES: CC(C(=O)N1CCN(c2ccc(OCCN)cc2)CC1)n1ncc2c1nc(N)n1nc(-c3ccco3)nc21]

To a stirred solution of the product of Step F in EtOH (10 mL) was added hydrazine hydrate (1 mL) at rt. The mixture was stirred at 70° C. for 3 hours. The reaction was cooled to RT and filtered. The filtrate was concentrated under reduced pressure. The residue was purified by Prep-HPLC to afford the title compound (120 mg, yield: 29.5%). ¹H NMR (400 MHz, DMSO-d6) δ 8.19 (s, 1H), 7.95 (s, 1H), 7.24 (d, J=4 Hz, 1H), 6.85-6.76 (m, 4H), 6.75-6.72 (m, 1H), 5.75 (q, J=6.7 Hz, 1H), 3.82 (t, J=6 Hz, 2H), 3.75-3.36 (m, 4H), 3.08-2.78 (m, 6H), 1.60 (d, J=8 Hz, 3H) ppm. MS: M/e 517 (M+1)⁺.

### Compound A8: 2-(5-amino-2-(furan-2-yl)-7H-pyrazolo[4,3-e][1,2,4]triazolo[1,5-c]pyrimidin-7-yl)-1-(4-(4-(2-(2-methoxyethoxy)ethoxy)phenyl)piperazin-1-yl)butan-1-one

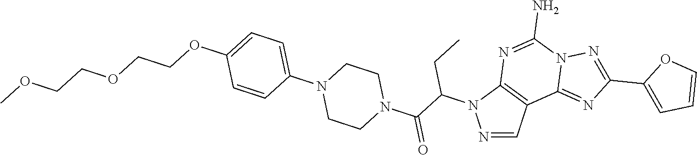 [SMILES: CCC(C(=O)N1CCN(c2ccc(OCCOCCOC)cc2)CC1)n1ncc2c1nc(N)n1nc(-c3ccco3)nc21]

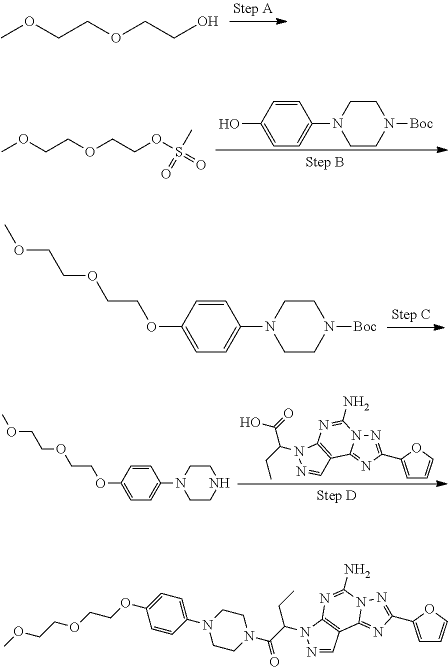

### Step A: 2-(2-methoxyethoxy)ethyl Methanesulfonate

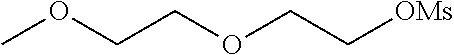

To a stirred mixture of 2-(2-methoxyethoxy)ethan-1-ol (10 g, 83.3 mmol) and TEA (26 g, 257 mmol) in DCM (300 mL) was added MsCl (14 g, 122.8 mmol). After the addition, the reaction mixture was stirred overnight. Most of solvent was removed to give the aqueous layer, washed with aq.NaHCO3 and extracted with EtOAc (200 mL×3). The combined organic layers were washed with brine, dried over Na₂SO₄, concentrated to give the target compound (11 g, 66.3%) as yellow oil. MS: M/e 199 (M+1)⁺.

### Step B: tert-butyl 4-(4-(2-(2-methoxyethoxy)ethoxy)phenyl)piperazine-1-carboxylate

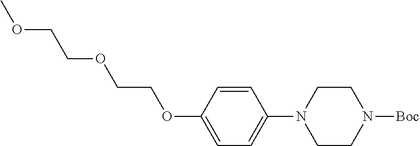 [SMILES: COCCOCCOc1ccc(N2CCN(C(=O)OC(C)(C)C)CC2)cc1]

To a stirred solution of tert-butyl 4-(4-hydroxyphenyl)piperazine-1-carboxylate (10.0 g, 36 mmol) in CH₃CN (200 mL) was added NaH (3.0 g, 125 mmol), the reaction mixture was stirred for 30 min at 50° C. Then 2-(2-methoxyethoxy)ethyl methanesulfonate (8.5 g, 43 mmol) was added into the reaction. After the addition, the reaction mixture was stirred overnight at 60° C. Most of solvent was removed and this residue was dissolved by H₂O (50 mL), then acidified to pH=6˜7 with aq.HCl and extracted with EtOAc (200 mL×3). The combined organic layers were washed with brine, dried over Na₂SO₄, concentrated and purified by column chromatography (petroleum ether/EtOAc=30% EtOAc) to give the target compound (13 g, 94.7%) as a yellow oil. MS: M/e 381 (M+1)⁺.

### Step C: 1-(4-(2-(2-methoxyethoxy)ethoxy)phenyl)piperazine

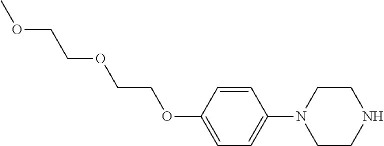 [SMILES: COCCOCCOc1ccc(N2CCNCC2)cc1]

To a stirred mixture of tert-butyl 4-(4-(2-(2-methoxyethoxy)ethoxy)phenyl)piperazine-1-carboxylate (13.0 g, 34.1 mmol) in DCM (100 mL) was added TFA (100 mL). After the addition, the reaction mixture was stirred for 3 hours at rt Most of solvent was removed and this residue was dissolved by H₂O (50 mL), then adjusted to pH=9˜10 with aq. NaOH and extracted with EtOAc (200 mL×3). The combined organic layers were washed with brine, dried over Na₂SO₄, concentrated to give 1-(4-(2-(2-methoxyethoxy)ethoxy)phenyl)piperazine (8 g, 83.5%) as a gray solid. MS: M/e 281 (M+1)⁺

### Step D: 2-(5-amino-2-(furan-2-yl)-7H-pyrazolo[4,3-e][1,2,4]triazolo[1,5-c]pyrimidin-7-yl)-1-(4-(4-(2-(2-methoxyethoxy)ethoxy)phenyl)piperazin-1-yl)butan-1-one

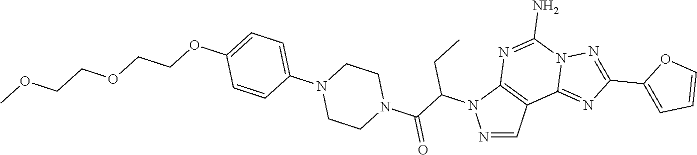 [SMILES: CCC(C(=O)N1CCN(c2ccc(OCCOCCOC)cc2)CC1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)butanoic acid (50 mg, 0.15 mmol), 1-(4-(2-(2-methoxyethoxy)ethoxy)phenyl)piperazine (65 mg, 0.23 mmol), HATU (86 mg, 0.23 mmol) and TEA (30 mg, 0.30 mmol) in DMF (5 mL) was stirred overnight. The reaction mixture was poured into H₂O (20 mL) and extracted with EtOAc (20 mL×3). The combined organic layers were washed with brine, dried over Na₂SO₄, concentrated and purified by column chromatography (petroleum ether/EtOAc=1:1˜100% EtOAc) to give the target compound (41 mg, 46.3%). ¹H NMR (400 MHz, DMSO-d6) δ 8.21 (s, 1H), 8.18 (br., 2H), 7.95 (s, 1H), 7.24 (d, J=3.4 Hz, 1H), 6.83-6.76 (m, 4H), 6.74 (dd, J=3.2, 1.7 Hz, 1H), 5.51 (dd, J=8.4, 6.0 Hz, 1H), 3.99-3.95 (m, 2H), 3.69-3.65 (m, 2H), 3.60-3.53 (m, 4H), 3.47-3.41 (m, 3H), 3.26-3.20 (m, 4H), 3.06-2.91 (m, 2H), 2.81 (s, 1H), 2.42 (s, 1H), 2.24-2.06 (m, 2H), 0.79 (t, J=7.3 Hz, 3H) ppm. MS: M/e 590 (M+1)⁺.

### Compound A9: 2-(5-amino-2-(furan-2-yl)-7H-pyrazolo[4,3-e][1,2,4]triazolo[1,5-c]pyrimidin-7-yl)-1-(4-(4-(2-(2-methoxyethoxy)ethoxy)phenyl)piperazin-1-yl)propan-1-one

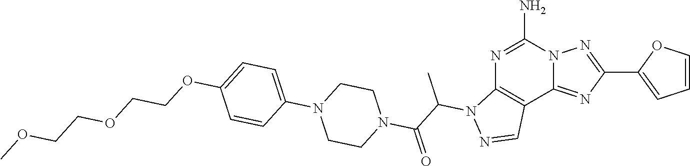 [SMILES: COCCOCCOc1ccc(N2CCN(C(=O)C(C)n3ncc4c3nc(N)n3nc(-c5ccco5)nc43)CC2)cc1]

A mixture of 1-(4-(2-(2-methoxyethoxy)ethoxy)phenyl)piperazine (30.8 mg, 0.11 mmol), 2-(5-amino-2-(furan-2-yl)-7H-pyrazolo[4,3-e][1,2,4]triazolo[1,5-c]pyrimidin-7-yl)propanoic acid (31 mg, 0.1 mmol), HATU (42 mg, 0.11 mmol) and Et₃N (0.2 mL, excess) in DMF (5 mL) was stirred at RT overnight. The reaction mixture was poured into water (15 mL) and extracted with EA (15 mL×3). The combined organic layers were washed with brine, dried over Na₂SO₄ and concentrated under reduced pressure. The residue was purified by prep-TLC (EA: 100%) to afford the title compound (8 mg, yield: 13.9%). ¹H NMR (400 MHz, DMSO-d6) δ 8.26-8.14 (m, 3H), 7.96 (s, 1H), 7.25 (d, J=4 Hz, 1H), 6.86-6.77 (m, 4H), 6.76-6.71 (m, 1H), 5.80-5.72 (m, 1H), 4.03-3.93 (m, 2H), 3.73-3.64 (m, 3H), 3.60-3.52 (m, 4H), 3.48-3.41 (m, 3H), 3.24 (s, 3H), 3.07-2.81 (m, 4H), 1.61 (d, J=8 Hz, 3H) ppm. MS: M/e 576 (M+1)⁺.

### Compound A10: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)hexan-1-one

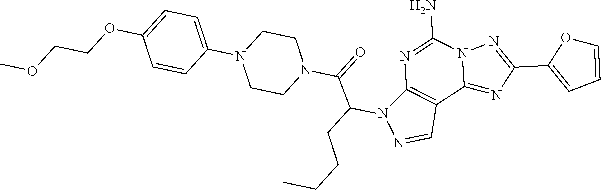 [SMILES: CCCCC(C(=O)N1CCN(c2ccc(OCCOC)cc2)CC1)n1ncc2c1nc(N)n1nc(-c3ccco3)nc21]

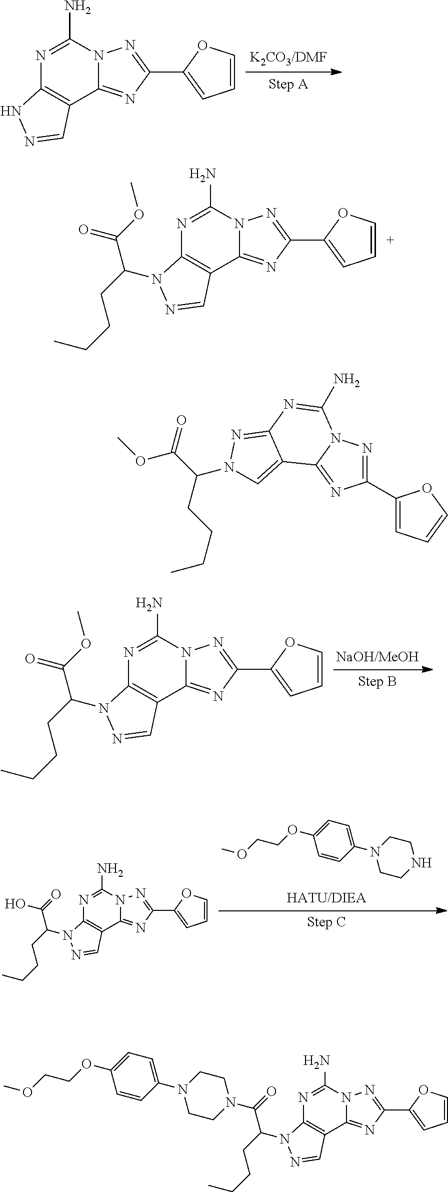

### Step A: methyl 2-(5-amino-2-(furan-2-yl)-7H-pyrazolo[4,3-e][1,2,4]triazolo[1,5-c]pyrimidin-7-yl)hexanoate and methyl 2-(5-amino-2-(furan-2-yl)-8H-pyrazolo[4,3-e][1,2,4]triazolo[1,5-c]pyrimidin-8-yl)hexanoate

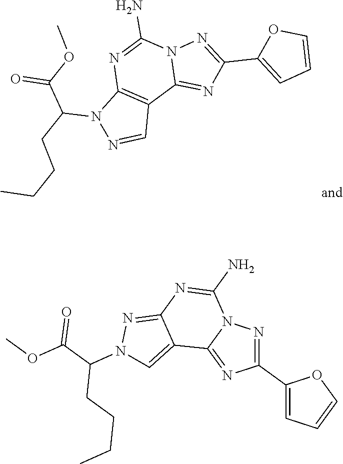 [SMILES: CCCCC(C(=O)OC)n1ncc2c1nc(N)n1nc(-c3ccco3)nc21 ; CCCCC(C(=O)OC)n1cc2c(nc(N)n3nc(-c4ccco4)nc23)n1]

A mixture of 2-(furan-2-yl)-7H-pyrazolo[4,3-e][1,2,4]triazolo[1,5-c]pyrimidin-5-amine (1.5 g, 6.2 mmol), methyl 2-bromohexanoate (1.5 g, 7.4 mmol) and K₂CO₃ (1.7 g, 12.4 mmol) in DMF (50 mL) was stirred at rt overnight. The solution was added with water (20 mL), extracted with ethyl aceate (40 mL) and washed with brine (40 mL). The organic layer was dried over Na₂SO₄, concentrated and purified by column chromatography (PE:EA=3:1 to 1:1) to get methyl 2-(5-amino-2-(furan-2-yl)-7H-pyrazolo[4,3-e][1,2,4]triazolo[1,5-c]pyrimidin-7-yl)hexanoate (380 mg, 16%). ¹H NMR (400 MHz, DMSO-d6) δ 8.24 (s, 1H), 8.16 (br.s, 2H), 7.95 (d, J=4.0 Hz, 1H), 7.24 (d, J=4.0 Hz, 1H), 6.74 (d, J=4.0 Hz, 1H), 5.38-5.34 (dd, J=12.0 Hz, 4.0 Hz 1H), 3.64 (s, 3H), 2.72-2.16 (m, 2H), 1.29-1.00 (m, 4H), 0.82-0.79 (m, 3H) ppm. MS: M/e 370 (M+1)⁺ and methyl 2-(5-amino-2-(furan-2-yl)-8H-pyrazolo[4,3-e][1,2,4]triazolo[1,5-c]pyrimidin-8-yl)hexanoate (310 mg, 13%). ¹H NMR (400 MHz, DMSO-d6) δ 8.77 (s, 1H), 7.95 (s, 1H), 7.68 (br.s, 2H), 7.20 (d, J=4.0 Hz, 1H), 6.75-6.73 (m, 1H), 5.31 (dd, J=12.0 Hz, 4.0 Hz 1H), 3.64 (s, 3H), 2.33-2.15 (m, 2H), 1.28-1.02 (m, 4H), 0.84-0.80 (m, 3H) ppm. MS: M/e 370 (M+1)⁺

### Step B: 2-(5-amino-2-(furan-2-yl)-7H-pyrazolo[4,3-e][1,2,4]triazolo[1,5-c]pyrimidin-7-yl)hexanoic Acid

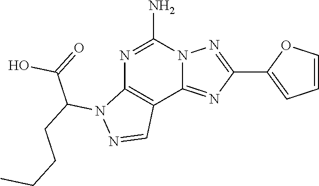 [SMILES: CCCCC(C(=O)O)n1ncc2c1nc(N)n1nc(-c3ccco3)nc21]

NaOH solution (160 mg, in 2 mL of water) was added to a solution of methyl 2-(5-amino-2-(furan-2-yl)-7H-pyrazolo[4,3-e][1,2,4]triazolo[1,5-c]pyrimidin-7-yl)hexanoate (380 mg, 1 mmol) in methanol (10 mL). The reaction mixture was stirred at rt overnight. The solution was concentrated, added with water (10 mL) and acidified with 1N HCl solution to pH=5. The precipitated solid was filtered and dried to get the desired product as a white solid (355 mg, 97%). MS: M/e 356 (M+1)⁺

### Step C: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)hexan-1-one

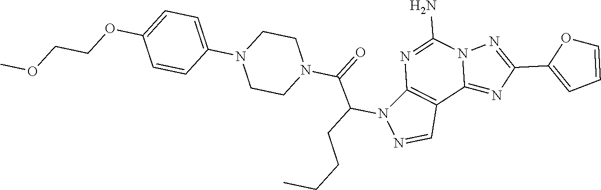 [SMILES: CCCCC(C(=O)N1CCN(c2ccc(OCCOC)cc2)CC1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)hexanoic acid (50 mg, 0.14 mmol), 1-(4-(2-methoxyethoxy)phenyl)piperazine (33 mg, 0.14 mmol), HATU (64 mg, 0.17 mmol) and DIEA (36 mg, 0.28 mmol) in DMF (5 mL) was stirred at rt for 2 hrs. The solution was added with water (10 mL), extracted with ethyl acetate (10 mL) and washed with brine (10 mL). The organic layer was dried, concentrated and purified by column chromatography (PE:EA=1:1 to EA) to get the desired product (40 mg, 49%). ¹H NMR (400 MHz, DMSO-d6) δ 8.20 (s, 1H), 8.17 (br.s, 2H), 7.95 (s, 1H), 7.23 (d, J=4.0 Hz, 1H), 6.80-6.73 (m, 5H), 5.56 (dd, J=8.0 Hz, 4.0 Hz 1H), 3.98-3.96 (m, 2H), 3.69-3.48 (m, 6H), 3.27 (s, 3H), 3.00-2.79 (m, 3H), 2.41-2.07 (m, 3H), 1.28-1.02 (m, 4H), 0.82 (t, J=8.0 Hz, 3H) ppm. MS: M/e 574 (M+1)⁺

Compound A10 was separated into two enantiomeric stereoisomers (Compound A10a, earlier peak, and Compound A10b, later peak) by chiral prep-HPLC. The chiral separation conditions are shown below.

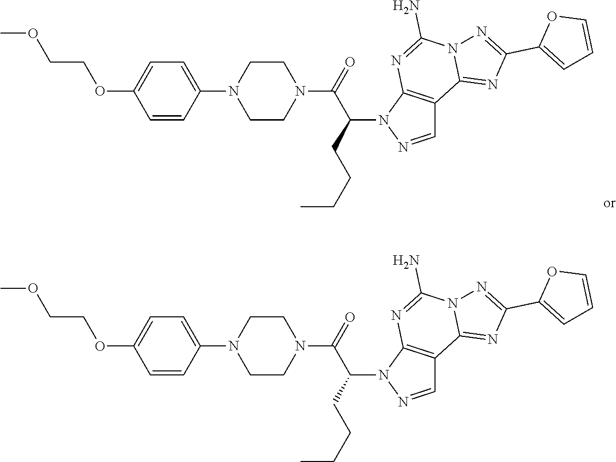 [SMILES: CCCC[C@@H](C(=O)N1CCN(c2ccc(OCCOC)cc2)CC1)n1ncc2c1nc(N)n1nc(-c3ccco3)nc21 ; CCCC[C@H](C(=O)N1CCN(c2ccc(OCCOC)cc2)CC1)n1ncc2c1nc(N)n1nc(-c3ccco3)nc21]

| Column | CHIRALPAK Cellulose-IC |
|---|---|
| Column size | 2 cm × 25 cm, 5 um |
| Injection | 0.5 mL |
| Mobile phase | (Hex:DCM = 50:50):EtOH = 50:50 |
| Flow rate | 16 ml/min |
| Wavelength | UV 220 nm |
| Temperature | 25° C. |
| Sample solution | 95.6 mg/ml in DCM:EtOH = 1:1 |
| Prep-HPLC equipment | Prep-Gilson-HPLC |

### Compound A11: 2-(5-amino-2-(furan-2-yl)-7H-pyrazolo[4,3-e][1,2,4]triazolo[1,5-c]pyrimidin-7-yl)-1-(4-(4-(2-(2-methoxyethoxy)ethoxy)phenyl)piperazin-1-yl)hexan-1-one

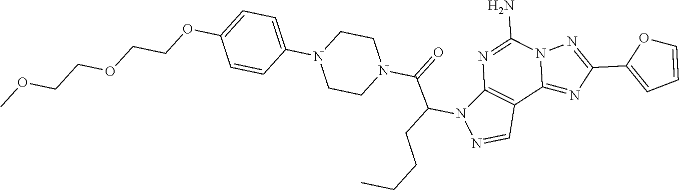 [SMILES: CCCCC(C(=O)N1CCN(c2ccc(OCCOCCOC)cc2)CC1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)hexanoic acid (320 mg, 0.9 mmol), 1-(4-(2-(2-methoxyethoxy)ethoxy)phenyl)piperazine (277 mg, 1.0 mmol), HATU (410 mg, 1.1 mmol) and DIEA (233 mg, 1.8 mmol) in DMF (10 mL) was stirred at rt overnight. The solution was added with water (10 mL), extracted with ethyl acetate (20 mL) and washed with brine (20 mL). The organic layer was dried, concentrated and purified by column chromatography (PE:EA=1:1 to EA) to get the desired product (350 mg, 63%). ¹H NMR (400 MHz, DMSO-d6) δ 8.20 (s, 1H), 8.17 (br.s, 2H), 7.95 (s, 1H), 7.23 (d, J=4.0 Hz, 1H), 6.80-6.73 (m, 5H), 5.56 (dd, J=8.0 Hz, 4.0 Hz, 1H), 3.97 (t, J=4.0 Hz, 2H), 3.68-3.42 (m, 13H), 3.00-2.73 (m, 3H), 2.41-2.07 (m, 3H), 1.35-1.19 (m, 3H), 1.03-1.02 (m, 1H), 0.81 (t, J=4.0 Hz, 3H) ppm. MS: M/e 618 (M+1)⁺

Compound A11 was separated into two enantiomeric stereoisomers (Compound A11a, earlier peak, and Compound A11b, later peak) by chiral prep-HPLC. The chiral separation conditions are shown below.

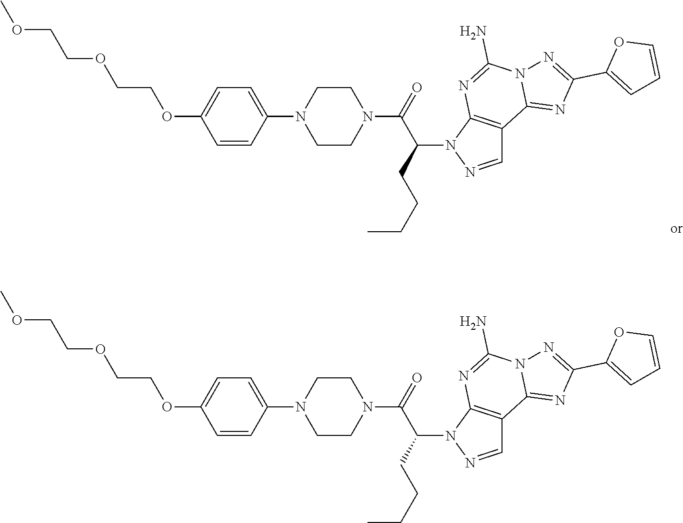 [SMILES: CCCC[C@@H](C(=O)N1CCN(c2ccc(OCCOCCOC)cc2)CC1)n1ncc2c1nc(N)n1nc(-c3ccco3)nc21 ; CCCC[C@H](C(=O)N1CCN(c2ccc(OCCOCCOC)cc2)CC1)n1ncc2c1nc(N)n1nc(-c3ccco3)nc21]

| Column | CHIRALPAK IC |
|---|---|
| Column size | 2 cm × 25 cm, 5 um |
| Injection | 1.2 mL |
| Mobile phase | (Hex:DCM = 3:1):EtOH = 50:50 |
| Flow rate | 16 ml/min |
| Wavelength | UV 220 nm |
| Temperature | 25° C. |
| Sample solution | 50.6 mg/ml in DCM:EtOH = 1:1 |
| Prep-HPLC equipment | Prep-Gilson-HPLC |

### Compound A12: 2-(5-amino-2-(furan-2-yl)-7H-pyrazolo[4,3-e][1,2,4]triazolo[1,5-c]pyrimidin-7-yl)-1-(4-(4-(2-(2-methoxyethoxy)ethoxy)phenyl)piperazin-1-yl)-3-methylbutan-1-one

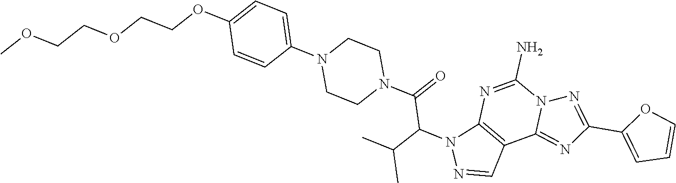 [SMILES: COCCOCCOc1ccc(N2CCN(C(=O)C(C(C)C)n3ncc4c3nc(N)n3nc(-c5ccco5)nc43)CC2)cc1]

To a mixture of 2-(5-amino-2-(furan-2-yl)-7H-pyrazolo[4,3-e][1,2,4]triazolo[1,5-c]pyrimidin-7-yl)-3-methylbutanoic acid (85 mg, 0.25 mmol), 1-(4-(2-(2-methoxyethoxy)ethoxy)phenyl)piperazine (77 mg, 0.27 mmol), DIEA (150 mg, 1.16 mmol) in DMF (3 mL) was added HATU (105 mg, 0.27 mmol) at rt and the mixture was stirred at rt for 4 hrs. 20 mL of EA was added and the mixture was washed with brine (10 mL×3), dried over Na₂SO₄ and concentrated. The resulting residue was purified by prep-TLC (EA/MeOH=20:1) to give the title product (66.0 mg, yield: 44%). ¹H NMR (400 MHz, DMSO-d6) δ 8.35-8.05 (m, 3H), 7.95 (s, 1H), 7.23 (d, J=3.2 Hz, 1H), 6.85-6.70 (m, 5H), 5.20 (d, J=10.0 Hz, 1H), 4.00-3.91 (m, 2H), 3.80-3.70 (m, 1H), 3.70-3.60 (m, 3H), 3.58-3.52 (m, 2H), 3.52-3.45 (m, 1H), 3.45-3.40 (m, 2H), 3.23 (s, 3H), 3.05-2.90 (m, 2H), 2.85-2.70 (m, 2H), 2.35-2.21 (m, 1H), 1.04 (d, J=6.4 Hz, 3H), 0.61 (d, J=6.8 Hz, 3H). MS: M/e 604 (M+1)⁺.

### Compound A13: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-phenylethan-1-one

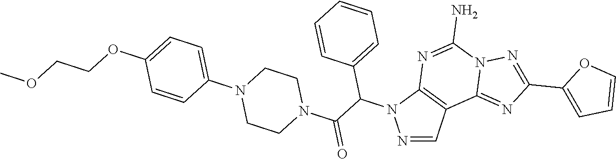 [SMILES: COCCOc1ccc(N2CCN(C(=O)C(c3ccccc3)n3ncc4c3nc(N)n3nc(-c5ccco5)nc43)CC2)cc1]

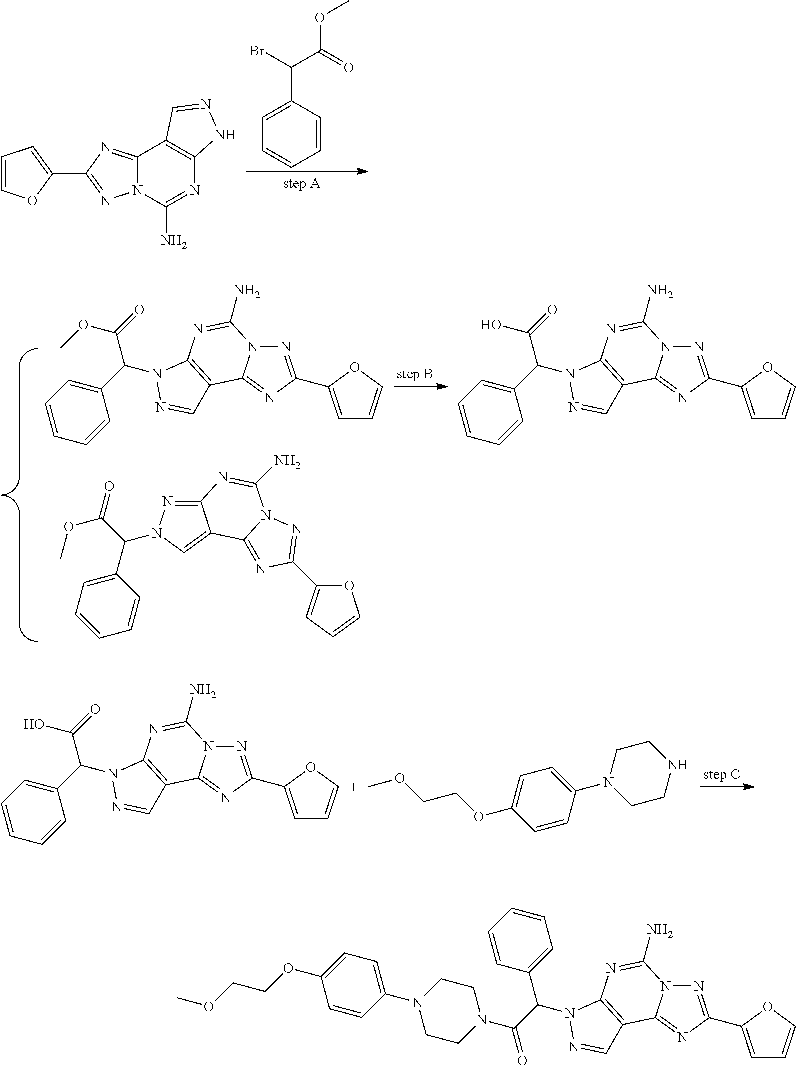

### Step A: methyl 2-(5-amino-2-(furan-2-yl)-7H-pyrazolo[4,3-e][1,2,4]triazolo[1,5-c]pyrimidin-7-yl)-2-phenylacetate and methyl 2-(5-amino-2-(furan-2-yl)-8H-pyrazolo[4,3-e][1,2,4]triazolo[1,5-c]pyrimidin-8-yl)-2-phenylacetate

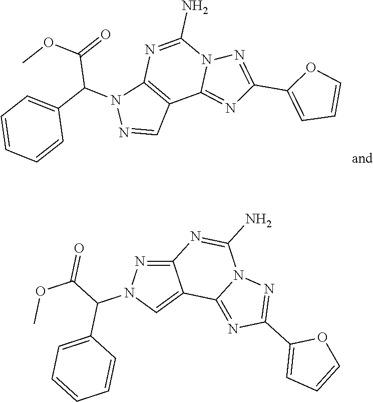 [SMILES: COC(=O)C(c1ccccc1)n1ncc2c1nc(N)n1nc(-c3ccco3)nc21 ; COC(=O)C(c1ccccc1)n1cc2c(nc(N)n3nc(-c4ccco4)nc23)n1]

To a stirred solution of 2-(furan-2-yl)-7H-pyrazolo[4,3-e][1,2,4]triazolo[1,5-c]pyrimidin-5-amine (1.0 g, 4.2 mmol) in DMF (50 mL) was added K₂CO₃ (1.3 g, 9.4 mmol) and methyl 2-bromo-2-phenylacetate (1.0 g, 4.4 mmol). After the addition, the reaction mixture was stirred overnight. The reaction mixture was poured into H₂O (50 mL) and extracted with EtOAc (100 mL×3). The combined organic layers were washed with brine, dried over Na₂SO₄, concentrated and purified by column chromatography (petroleum ether/EtOAc=2:1˜1:3) to give methyl 2-(5-amino-2-(furan-2-yl)-7H-pyrazolo[4,3-e][1,2,4]triazolo[1,5-c]pyrimidin-7-yl)-2-phenylacetate (200 mg, 24.5%) and methyl 2-(5-amino-2-(furan-2-yl)-8H-pyrazolo[4,3-e][1,2,4]triazolo[1,5-c]pyrimidin-8-yl)-2-phenylacetate (360 mg, 44.0%) as white solids. MS: M/e 390 (M+1)⁺.

### Step B: 2-(5-amino-2-(furan-2-yl)-7H-pyrazolo[4,3-e][1,2,4]triazolo[1,5-c]pyrimidin-7-yl)-2-phenylacetic Acid

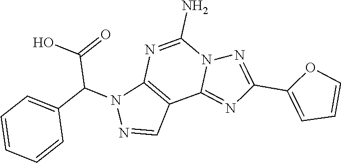 [SMILES: Nc1nc2c(cnn2C(C(=O)O)c2ccccc2)c2nc(-c3ccco3)nn12]

To a stirred mixture of methyl 2-(5-amino-2-(furan-2-yl)-7H-pyrazolo[4,3-e][1,2,4]triazolo[1,5-c]pyrimidin-7-yl)-2-phenylacetate (200 mg, 0.51 mmol) in MeOH/H₂O (3 mL/1 mL) was added aq.NaOH (2.0 M, 2 mL). After the addition, the reaction mixture was stirred overnight. Most of solvent was removed to give the aqueous layer, then acidified to pH=3˜4 with aq.HCl and filtered, the cake was collected, dried to give the target compound (150 mg, 78.2%) as a white solid. MS: M/e 376 (M+1)⁺.

### Step C: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-phenylethan-1-one

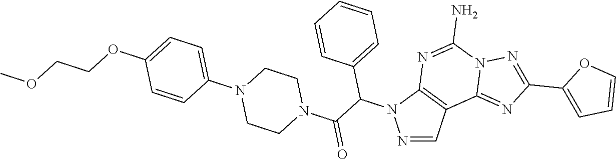 [SMILES: COCCOc1ccc(N2CCN(C(=O)C(c3ccccc3)n3ncc4c3nc(N)n3nc(-c5ccco5)nc43)CC2)cc1]

A mixture of the product of step B (50 mg, 0.13 mmol), 1-(4-(2-methoxyethoxy)phenyl)piperazine (55 mg, 0.23 mmol), HATU (75 mg, 0.20 mmol) and TEA (40 mg, 0.39 mmol) in DMF (10 mL) was stirred for 5 hours at RT. The reaction mixture was poured into H₂O (20 mL) and extracted with EtOAc (50 mL×3). The combined organic layers were washed with brine, dried over Na₂SO₄, concentrated and purified by column chromatography (petroleum ether/EtOAc=1:1˜100% EtOAc) to give the target compound (42 mg, 54.4%). ¹H NMR (400 MHz, DMSO-d6) δ 8.17 (s, 1H), 7.92 (s, 1H), 7.70 (br., 2H), 7.59-7.43 (m, 5H), 7.15 (d, J=3.4 Hz, 1H), 7.11 (s, 1H), 6.86-6.77 (m, 4H), 6.75-6.69 (m, 1H), 4.03-3.95 (m, 2H), 3.80-3.71 (m, 1H), 3.71-3.57 (m, 3H), 3.50-3.40 (m, 1H), 3.34 (s, 2H), 3.28 (s, 3H), 3.10-3.01 (m, 2H), 2.98-2.87 (m, 2H) ppm. MS: M/e 594 (M+1)⁺.

Compound A13 was separated into two enantiomeric stereoisomers (Compound A13a, earlier peak, and Compound A13b, later peak) by chiral prep-HPLC. The chiral separation conditions are shown below.

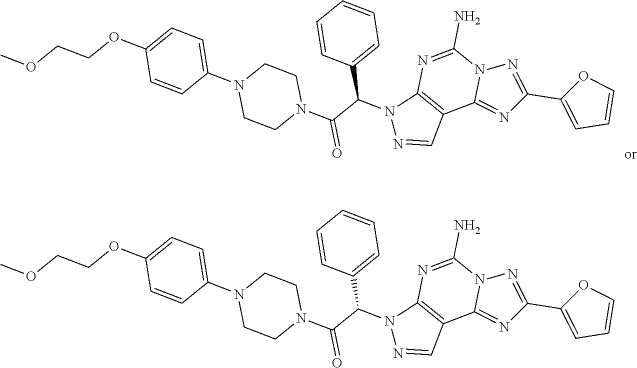 [SMILES: COCCOc1ccc(N2CCN(C(=O)[C@@H](c3ccccc3)n3ncc4c3nc(N)n3nc(-c5ccco5)nc43)CC2)cc1 ; COCCOc1ccc(N2CCN(C(=O)[C@H](c3ccccc3)n3ncc4c3nc(N)n3nc(-c5ccco5)nc43)CC2)cc1]

| Column | CHIRALPAK IC |
|---|---|
| Column size | 2 cm × 25 cm, 5 um |
| Injection | 4.8 ml |
| Mobile phase | CO₂:(MeOH:DCM = 3:1) = 50:50 |
| Flow rate | 40 ml/min |
| Wavelength | UV 220 nm |
| Temperature | 25° C. |
| Sample solution | 6.9 mg/ml in MeOH:DCM = 1:1 |
| Prep-SFC equipment | Prep-SFC-80 |

### Compound A14: 2-(5-amino-2-(furan-2-yl)-7H-pyrazolo[4,3-e][1,2,4]triazolo[1,5-c]pyrimidin-7-yl)-1-(4-(4-(2-(2-methoxyethoxy)ethoxy)phenyl)piperazin-1-yl)-2-phenylethan-1-one

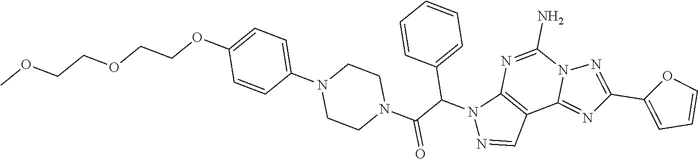 [SMILES: COCCOCCOc1ccc(N2CCN(C(=O)C(c3ccccc3)n3ncc4c3nc(N)n3nc(-c5ccco5)nc43)CC2)cc1]

A mixture of the product of step B (50 mg, 0.13 mmol), 1-(4-(2-(2-methoxyethoxy)ethoxy)phenyl)piperazine (40 mg, 0.14 mmol), HATU (84 mg, 0.22 mmol) and TEA (40 mg, 0.39 mmol) in DMF (10 mL) was stirred for 4 hours at RT. The reaction mixture was poured into H₂O (20 mL) and extracted with EtOAc (50 mL×3). The combined organic layers were washed with brine, dried over Na₂SO₄, concentrated and purified by column chromatography (petroleum ether/EtOAc=1:1˜100% EtOAc) to give the target compound (33 mg, 39.9%). ¹H NMR (400 MHz, DMSO-d6) δ 8.20 (s, 1H), 8.16 (br., 2H), 7.95 (s, 1H), 7.43-7.31 (m, 5H), 7.24 (d, J=2.9 Hz, 1H), 6.86 (s, 1H), 6.81-6.78 (m, 4H), 6.74 (dd, J=3.4, 1.8 Hz, 1H), 4.01-3.94 (m, 2H), 3.70-3.65 (m, 2H), 3.55 (dd, J=5.7, 3.8 Hz, 2H), 3.44 (dd, J=5.7, 3.7 Hz, 3H), 3.35 (s, 3H), 3.23 (s, 3H), 3.05-2.87 (m, 2H), 2.71-2.54 (m, 2H) ppm. MS: M/e 638 (M+1)⁺.

### Compound A15: 2-(5-amino-2-(furan-2-yl)-7H-pyrazolo[4,3-e][1,2,4]triazolo[1,5-c]pyrimidin-7-yl)-2-cyclopropyl-1-(4-(4-(2-methoxyethoxy)phenyl)piperazin-1-yl)ethan-1-one

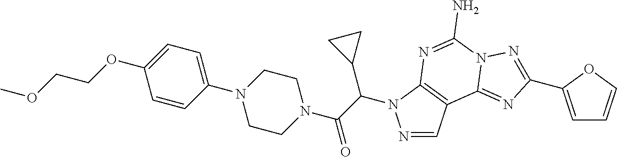 [SMILES: COCCOc1ccc(N2CCN(C(=O)C(C3CC3)n3ncc4c3nc(N)n3nc(-c5ccco5)nc43)CC2)cc1]

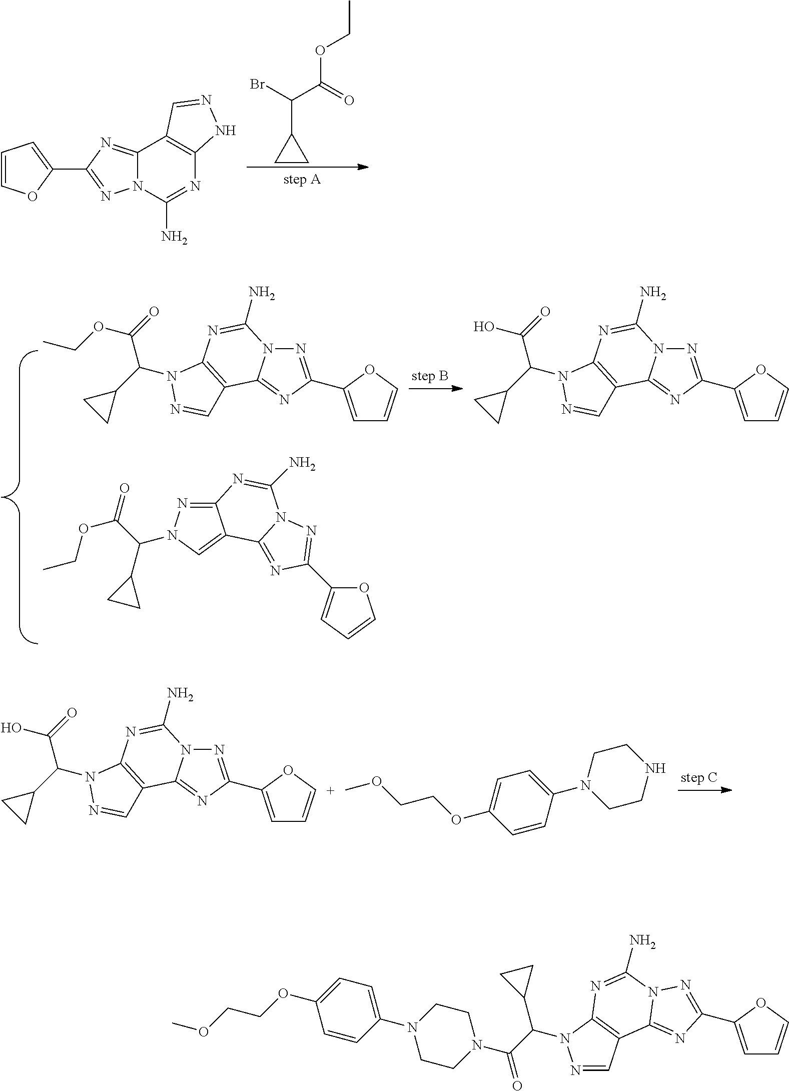

### Step A: ethyl 2-(5-amino-2-(furan-2-yl)-7H-pyrazolo[4,3-e][1,2,4]triazolo[1,5-c]pyrimidin-7-yl)-2-cyclopropylacetate

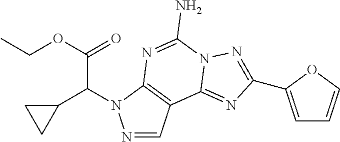 [SMILES: CCOC(=O)C(C1CC1)n1ncc2c1nc(N)n1nc(-c3ccco3)nc21]

To a stirred solution of 2-(furan-2-yl)-7H-pyrazolo[4,3-e][1,2,4]triazolo[1,5-c]pyrimidin-5-amine (1.0 g, 4.2 mmol) in DMF (50 mL) was added K₂CO₃ (1.3 g, 9.4 mmol) and ethyl 2-bromo-2-cyclopropylacetate (1.2 g, 5.8 mmol). After the addition, the reaction mixture was stirred overnight at 60° C. The reaction mixture was poured into H₂O (50 mL) and extracted with EtOAc (100 mL×3). The combined organic layers were washed with brine, dried over Na₂SO₄, concentrated and purified by column chromatography (petroleum ether/EtOAc=2:1˜1:2) to give ethyl 2-(5-amino-2-(furan-2-yl)-7H-pyrazolo[4,3-e][1,2,4]triazolo[1,5-c]pyrimidin-7-yl)-2-cyclopropylacetate (400 mg, 51.4%) and ethyl 2-(5-amino-2-(furan-2-yl)-8H-pyrazolo[4,3-e][1,2,4]triazolo[1,5-c]pyrimidin-8-yl)-2-cyclopropylacetate (500 mg, 64.7%) as white solids. MS: M/e 368 (M+1)⁺.

### Step B: 2-(5-amino-2-(furan-2-yl)-7H-pyrazolo[4,3-e][1,2,4]triazolo[1,5-c]pyrimidin-7-yl)-2-cyclopropylacetic Acid

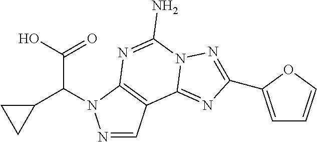 [SMILES: Nc1nc2c(cnn2C(C(=O)O)C2CC2)c2nc(-c3ccco3)nn12]

To a stirred mixture of ethyl 2-(5-amino-2-(furan-2-yl)-7H-pyrazolo[4,3-e][1,2,4]triazolo[1,5-c]pyrimidin-7-yl)-2-cyclopropylacetate (400 mg, 1.1 mmol) in MeOH/H₂O (9 mL/2 mL) was added aq.NaOH (4.0 M, 2 mL). After the addition, the reaction mixture was stirred for 3 hours at RT. Most of solvent was removed to give the aqueous layer, then acidified to pH=3˜4 with aq.HCl and filtered, the cake was collected, dried to give the target compound (200 mg, 53.5%) as a white solid. MS: M/e 340 (M+1)⁺.

### Step C: 2-(5-amino-2-(furan-2-yl)-7H-pyrazolo[4,3-e][1,2,4]triazolo[1,5-c]pyrimidin-7-yl)-2-cyclopropyl-1-(4-(4-(2-methoxyethoxy)phenyl)piperazin-1-vl ethan-1-one

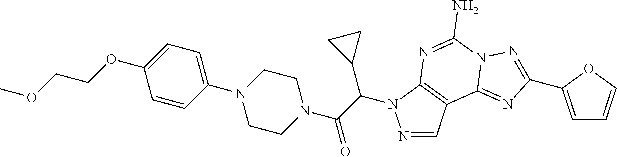 [SMILES: COCCOc1ccc(N2CCN(C(=O)C(C3CC3)n3ncc4c3nc(N)n3nc(-c5ccco5)nc43)CC2)cc1]

A mixture of the product of step B (50 mg, 0.15 mmol), 1-(4-(2-methoxyethoxy)phenyl)piperazine (45 mg, 0.19 mmol), HATU (84 mg, 0.22 mmol) and TEA (30 mg, 0.30 mmol) in DMF (10 mL) was stirred overnight at RT. The reaction mixture was poured into H₂O (20 mL) and extracted with EtOAc (50 mL×3). The combined organic layers were washed with brine, dried over Na₂SO₄, concentrated and purified by column chromatography (petroleum ether/EtOAc=1:1˜100% EtOAc) to give the target compound (28 mg, 33.5%). ¹H NMR (400 MHz, DMSO-d6) δ 8.21 (s, 1H), 8.18 (d, 2H), 7.95 (s, 1H), 7.24 (d, J=2.6 Hz, 1H), 6.79-6.76 (m, 4H), 6.74 (s, 1H), 4.87 (d, J=9.5 Hz, 1H), 4.05-3.89 (m, 2H), 3.81-3.68 (m, 1H), 3.64-3.56 (m, 3H), 3.56-3.38 (m, 2H), 3.27 (s, 3H), 3.01 (s, 1H), 2.93-2.74 (m, 2H), 2.35-2.23 (m, 1H), 1.86-1.74 (m, 1H), 0.94-0.82 (m, 1H), 0.74-0.61 (m, 1H), 0.57-0.39 (m, 3H). MS: M/e 558 (M+1)⁺.

### Compound A16: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-4-methylpentan-1-one

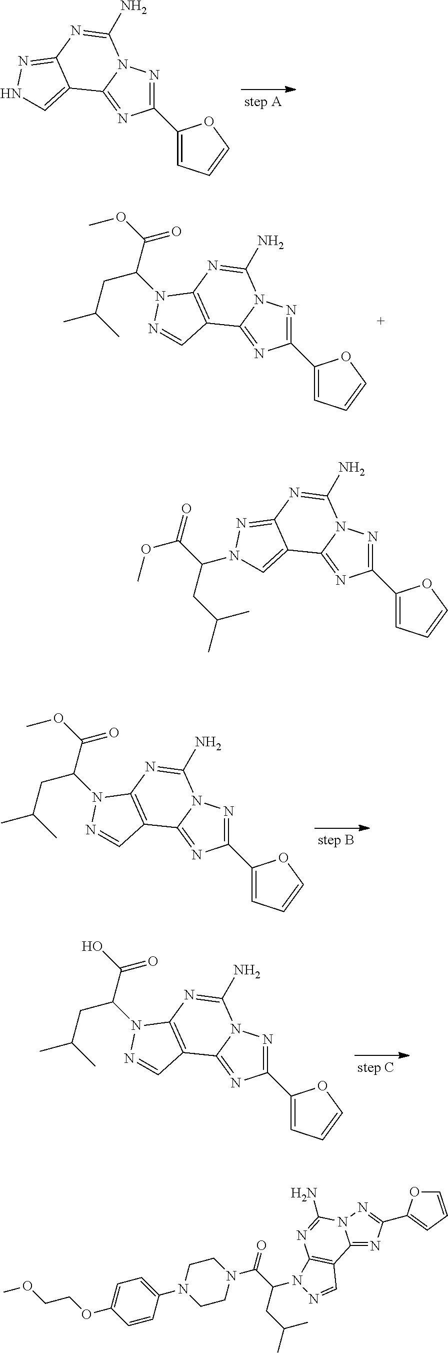 [SMILES: Nc1nc2n[nH]cc2c2nc(-c3ccco3)nn12 ; COC(=O)C(CC(C)C)n1ncc2c1nc(N)n1nc(-c3ccco3)nc21 ; COC(=O)C(CC(C)C)n1cc2c(nc(N)n3nc(-c4ccco4)nc23)n1 ; COC(=O)C(CC(C)C)n1ncc2c1nc(N)n1nc(-c3ccco3)nc21 ; CC(C)CC(C(=O)O)n1ncc2c1nc(N)n1nc(-c3ccco3)nc21 ; COCCOc1ccc(N2CCN(C(=O)C(CC(C)C)n3ncc4c3nc(N)n3nc(-c5ccco5)nc43)CC2)cc1]

### Step A: methyl 2-(5-amino-2-(furan-2-yl)-7H-pyrazolo[4,3-e][1,2,4]triazolo[1,5-c]pyrimidin-7-yl)-4-methylpentanoate and methyl 2-(5-amino-2-(furan-2-yl)-8H-pyrazolo[4,3-e][1,2,4]triazolo[1,5-c]pyrimidin-8-yl)-4-methylpentanoate

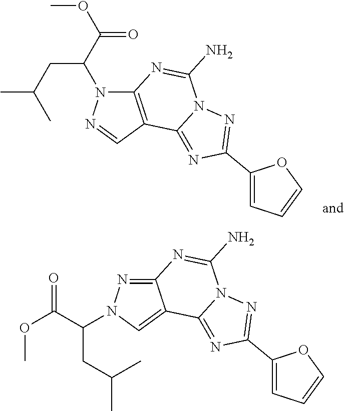

To a stirred solution of 2-(furan-2-yl)-7H-pyrazolo[4,3-e][1,2,4]triazolo[1,5-c]pyrimidin-5-amine (482 mg, 2 mmol) in DMF (10 mL) was added K₂CO₃ (552 mg, 4 mmol) and methyl 2-bromo-4-methylpentanoate (418 mg, 2 mmol). After the addition, the reaction mixture was stirred for a weekend. The reaction mixture was poured into H₂O (20 mL) and extracted with EtOAc (15 mL×3). The combined organic layers were washed with brine, dried over Na₂SO₄, concentrated and purified by column chromatography (petroleum ether/EtOAc=3:1-1:1) to give methyl 2-(5-amino-2-(furan-2-yl)-7H-pyrazolo[4,3-e][1,2,4]triazolo[1,5-c]pyrimidin-7-yl)-4-methylpentanoate (280 mg, 37.9%) and methyl 2-(5-amino-2-(furan-2-yl)-8H-pyrazolo[4,3-e][1,2,4]triazolo[1,5-c]pyrimidin-8-yl)-4-methylpentanoate (213 mg, 28.80%) as white solids. MS: M/e 370 (M+1)⁺.

### Step B: 2-(5-amino-2-(furan-2-yl)-7H-pyrazolo[4,3-e][1,2,4]triazolo[1,5-c]pyrimidin-7-yl)-4-methylpentanoic Acid

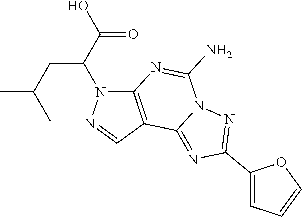 [SMILES: CC(C)CC(C(=O)O)n1ncc2c1nc(N)n1nc(-c3ccco3)nc21]

To a stirred mixture of methyl 2-(5-amino-2-(furan-2-yl)-7H-pyrazolo[4,3-e][1,2,4]triazolo[1,5-c]pyrimidin-7-yl)-4-methylpentanoate (200 mg, 0.54 mmol) in MeOH/H₂O (10 mL/5 mL) was added aq.NaOH (2.0 M, 4 mL). After the addition, the reaction mixture was stirred overnight. Most of solvent was removed to give the aqueous layer, then acidified to pH=3˜4 with aq.HCl and filtered, the cake was collected, dried to give the target compound (180 mg, 93.8%) as a white solid. MS: M/e 356 (M+1)⁺.

### Step C: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-4-methylpentan-1-one

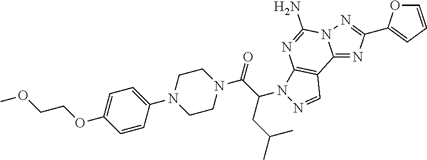 [SMILES: COCCOc1ccc(N2CCN(C(=O)C(CC(C)C)n3ncc4c3nc(N)n3nc(-c5ccco5)nc43)CC2)cc1]

A mixture of the product of step B (50 mg, 0.14 mmol), 1-(4-(2-methoxyethoxy)phenyl)piperazine (33 mg, 0.14 mmol), HATU (64 mg, 0.168 mmol) and DIPEA (36 mg, 0.28 mmol) in DMF (5 mL) was stirred for 2 hours at RT. The reaction mixture was poured into H₂O (20 mL) and extracted with EtOAc (20 mL×3). The combined organic layers were washed with brine, dried over Na₂SO₄, concentrated and purified by column chromatography (petroleum ether/EtOAc=1:1˜100% EtOAc) to give the target compound (30 mg, 37.4%). ¹H NMR (400 MHz, DMSO-d6) δ 8.21 (s, 1H), 8.18 (s, 2H), 7.95 (d, J=0.8 Hz, 1H), 7.24 (d, J=3.2 Hz, 1H), 6.83-6.72 (m, 5H), 5.64 (m, 1H), 4.00-3.93 (m, 2H), 3.70 (m, 1H), 3.62-3.43 (m, 5H), 3.27 (s, 3H), 3.02 (m, 2H), 2.77 (m, 1H), 2.45-2.35 (m, 1H), 2.29-2.17 (m, 1H), 1.86-1.83 (m, 1H), 1.32-1.21 (m, 1H), 0.95 (d, J=6.4 Hz, 3H), 0.83 (d, J=6.4 Hz, 3H) ppm. MS: M/e 574 (M+1)⁺.

Compound A16 was separated into two enantiomeric stereoisomers (Compound A16a, earlier peak, and Compound A16b, later peak) by chiral prep-HPLC. The chiral separation conditions are shown below.

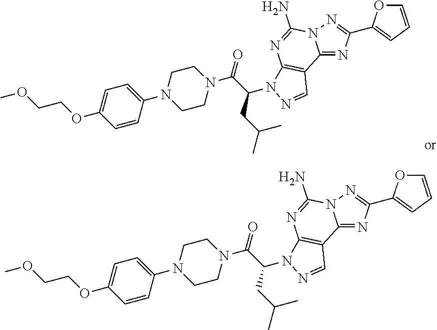 [SMILES: COCCOc1ccc(N2CCN(C(=O)[C@H](CC(C)C)n3ncc4c3nc(N)n3nc(-c5ccco5)nc43)CC2)cc1 ; COCCOc1ccc(N2CCN(C(=O)[C@@H](CC(C)C)n3ncc4c3nc(N)n3nc(-c5ccco5)nc43)CC2)cc1]

| Column | CHIRALPAK IC |
|---|---|
| Column size | 2 cm × 25 cm, 5 um |
| Injection | 1.5 mL |
| Mobile phase | (Hex:DCM = 3:1):EtOH = 50:50 |
| Flow rate | 16 ml/min |
| Wavelength | UV 220 nm |
| Temperature | 25° C. |
| Sample solution | 49.4 mg/ml in DCM:EtOH = 1:1 |
| Prep-HPLC equipment | Prep-Gilson-HPLC |

### Compound A17: 2-(5-amino-2-(furan-2-yl)-7H-pyrazolo[4,3-e][1,2,4]triazolo[1,5-c]pyrimidin-7-yl)-1-(4-(4-(2-(2-methoxyethoxy)ethoxy)phenyl)piperazin-1-yl)-4-methylpentan-1-one

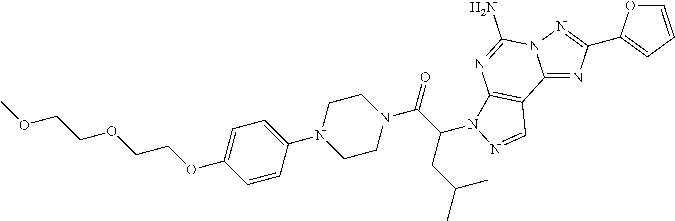 [SMILES: COCCOCCOc1ccc(N2CCN(C(=O)C(CC(C)C)n3ncc4c3nc(N)n3nc(-c5ccco5)nc43)CC2)cc1]

A mixture of 2-(5-amino-2-(furan-2-yl)-7H-pyrazolo[4,3-e][1,2,4]triazolo[1,5-c]pyrimidin-7-yl)-4-methylpentanoic acid (50 mg, 0.14 mmol), 1-(4-(2-(2-methoxyethoxy)ethoxy)phenyl)piperazine (39.4 mg, 0.14 mmol), HATU (64 mg, 0.168 mmol) and DIPEA (36 mg, 0.28 mmol) in DMF (5 mL) was stirred for 2 hours at RT. The reaction mixture was poured into H₂O (20 mL) and extracted with EtOAc (20 mL×3). The combined organic layers were washed with brine, dried over Na₂SO₄, concentrated and purified by column chromatography (petroleum ether/EtOAc=1:1˜100% EtOAc) to give the target compound (30 mg, 34.7%). ¹H NMR (400 MHz, DMSO-d6) δ 8.21 (s, 1H), 8.18 (s, 2H), 7.95 (d, J=0.8 Hz, 1H), 7.24 (d, J=3.2 Hz, 1H), 6.85-6.75 (m, 4H), 6.74 (m, 1H), 5.64 (dd, J=10.0, 4.8 Hz, 1H), 4.00-3.90 (m, 2H), 3.68 (m, 3H), 3.64-3.51 (m, 4H), 3.48-3.40 (m, 3H), 3.23 (s, 3H), 3.11-2.92 (m, 2H), 2.77 (m, 1H), 2.46-2.35 (m, 1H), 2.30-2.16 (m, 1H), 1.85 (m, 1H), 1.28 (m, 1H), 0.95 (d, J=6.4 Hz, 3H), 0.83 (d, J=6.4 Hz, 3H) ppm. MS: M/e 618 (M+1)⁺.

Compound A17 was separated into two enantiomeric stereoisomers (Compound A17a, earlier peak, and Compound A17b, later peak) by chiral prep-HPLC. The chiral separation conditions are shown below.

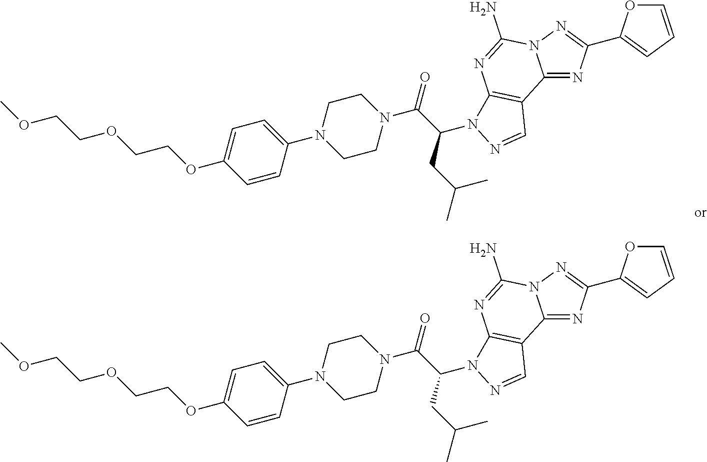 [SMILES: COCCOCCOc1ccc(N2CCN(C(=O)[C@H](CC(C)C)n3ncc4c3nc(N)n3nc(-c5ccco5)nc43)CC2)cc1 ; COCCOCCOc1ccc(N2CCN(C(=O)[C@@H](CC(C)C)n3ncc4c3nc(N)n3nc(-c5ccco5)nc43)CC2)cc1]

| Column | CHIRALPAK IC |
|---|---|
| Column size | 2 cm × 25 cm, 5 um |
| Injection | 2 mL |
| Mobile phase | (Hex:DCM = 3:1):EtOH = 50:50 |
| Flow rate | 16 ml/min |
| Wavelength | UV 220 nm |
| Temperature | 25° C. |
| Sample solution | 43.1 mg/ml in DCM:EtOH = 1:1 |
| Prep-HPLC equipment | Prep-Gilson-HPLC |

### Compound A18: 2-(5-amino-2-(furan-2-yl)-7H-pyrazolo[4,3-e][1,2,4]triazolo[1,5-c]pyrimidin-7-yl)-1-(4-(4-(2-(dimethylamino)ethoxy)phenyl)piperazin-1-yl)propan-1-one

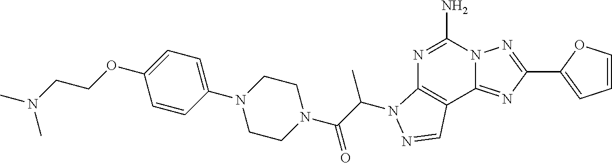 [SMILES: CC(C(=O)N1CCN(c2ccc(OCCN(C)C)cc2)CC1)n1ncc2c1nc(N)n1nc(-c3ccco3)nc21]

A mixture of N,N-dimethyl-2-(4-(piperazin-1-yl)phenoxy)ethan-1-amine hydrochloride (100 mg, 0.35 mmol), 2-(5-amino-2-(furan-2-yl)-7H-pyrazolo[4,3-e][1,2,4]triazolo[1,5-c]pyrimidin-7-yl)propanoic acid (100 mg, 0.32 mmol), HATU (134 mg, 0.35 mmol) and DIEA (1 mL, excess) in DMF (5 mL) was stirred at RT overnight. The reaction mixture was poured into water (10 mL) and the solid was precipitated from the system. The solid was filtered and purified by prep-HPLC to afford the title compound (5 mg, yield: 3%). ¹H NMR (400 MHz, DMSO-d6) δ 8.33-8.08 (m, 3H), 7.96 (s, 1H), 7.67-7.36 (m, 2H), 7.24 (d, J=4.0 Hz, 1H), 7.07 (d, J=8.0 Hz, 2H), 6.74 (s, 1H), 5.86-5.74 (m, 1H), 4.42-4.31 (m, 4H), 4.03-3.69 (m, 4H), 3.54-3.32 (m, 4H), 2.86-2.74 (m, 6H), 1.64 (d, J=8.0 Hz, 3H) ppm. MS: M/e 545 (M+1)⁺.

### Compound A19: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-3-phenylpropan-1-one

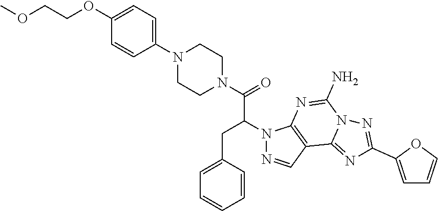 [SMILES: COCCOc1ccc(N2CCN(C(=O)C(Cc3ccccc3)n3ncc4c3nc(N)n3nc(-c5ccco5)nc43)CC2)cc1]

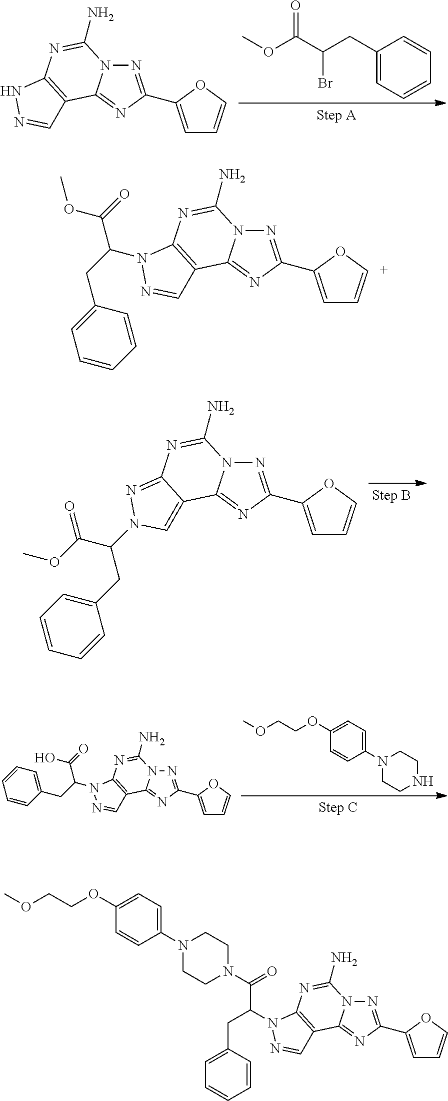

### Step A: methyl 2-(5-amino-2-(furan-2-yl)-7H-pyrazolo[4,3-e][1,2,4]triazolo[1,5-c]pyrimidin-7-yl)-3-phenylpropanoate and methyl 2-(5-amino-2-(furan-2-yl)-8H-pyrazolo[4,3-e][1,2,4]triazolo[1,5-c]pyrimidin-8-yl)-3-phenylpropanoate

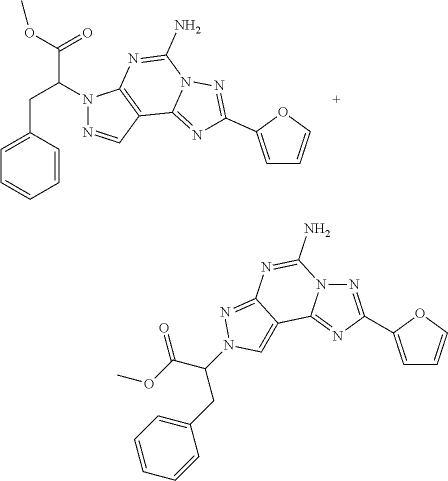 [SMILES: COC(=O)C(Cc1ccccc1)n1ncc2c1nc(N)n1nc(-c3ccco3)nc21 ; COC(=O)C(Cc1ccccc1)n1cc2c(nc(N)n3nc(-c4ccco4)nc23)n1]

A mixture of 2-(furan-2-yl)-7H-pyrazolo[4,3-e][1,2,4]triazolo[1,5-c]pyrimidin-5-amine (300 mg, 1.25 mmol), methyl 2-bromo-3-phenylpropanoate (330 mg, 1.36 mmol) and K₂CO₃ (400 mg, 2.90 mmol) in DMF (7 mL) was heated at rt for 20 hrs and 50° C. for 5 hrs. The mixture was diluted with EA (30 mL) and the suspension was filtered. The filtrate was washed with brine (15 mL×3), dried over Na₂SO₄ and concentrated. The resulting residue was purified by prep-TLC to give methyl 2-(5-amino-2-(furan-2-yl)-7H-pyrazolo[4,3-e][1,2,4]triazolo[1,5-c]pyrimidin-7-yl)-3-phenylpropanoate (130 mg), MS: M/e 404 (M+1)⁺, and methyl 2-(5-amino-2-(furan-2-yl)-8H-pyrazolo[4,3-e][1,2,4]triazolo[1,5-c]pyrimidin-8-yl)-3-phenylpropanoate (125 mg) as white solid. MS: M/e 404 (M+1)⁺.

### Step B: 2-(5-amino-2-(furan-2-yl)-7H-pyrazolo[4,3-e][1,2,4]triazolo[1,5-c]pyrimidin-7-yl)-3-phenylpropanoic Acid

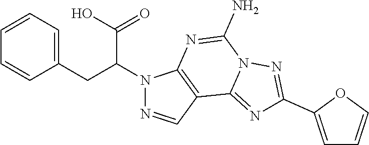 [SMILES: Nc1nc2c(cnn2C(Cc2ccccc2)C(=O)O)c2nc(-c3ccco3)nn12]

To a stirred solution of the methyl 2-(5-amino-2-(furan-2-yl)-7H-pyrazolo[4,3-e][1,2,4]triazolo[1,5-c]pyrimidin-7-yl)-3-phenylpropanoate (120 mg, 0.25 mmol) in MeOH (5 mL) was added aqueous solution of NaOH (2 M, 2 mL) at rt and the resulting mixture was stirred for 2 hrs. The mixture was neutralized by HCl (2 M) and concentrated to dryness, resulting as a white solid. 20 mL of a mixed solvent (CH₂Cl₂/MeOH=3:1) was added and stirred for 10 min. The suspension was filtered and the filtrate was concentrated to give the title product (95 mg, 96%) as a white solid. MS: M/e390 (M+1)⁺.

### Step C: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-3-phenylpropan-1-one

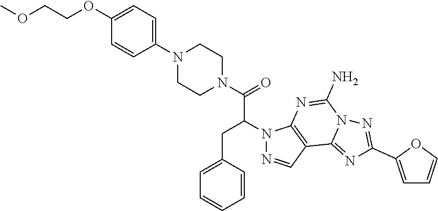 [SMILES: COCCOc1ccc(N2CCN(C(=O)C(Cc3ccccc3)n3ncc4c3nc(N)n3nc(-c5ccco5)nc43)CC2)cc1]

To a mixture of the product from step B (45 mg, 0.12 mmol), 1-(4-(2-methoxyethoxy)phenyl)piperazine (45 mg, 0.19 mmol), DIEA (70 mg, 0.54 mmol) in DMF (2 mL) was added HATU (75 mg, 0.20 mmol) at rt and the mixture was stirred at rt for 16 hrs. 30 mL of EA was added and the mixture was washed with brine (15 mL×3), dried over Na₂SO₄ and concentrated. The resulting residue was purified by prep-TLC (CH₂Cl₂/EA/MeOH=10:10:1), and the resulting product was purified by prep-HPLC to give the title product (32.0 mg, yield: 44%). ¹H NMR (400 MHz, DMSO-d6) δ 8.22 (s, 1H), 8.15-8.0 (m, 2H), 7.94 (s, 1H), 7.22 (d, J=3.2 Hz, 1H), 7.18-7.11 (m, 2H), 7.11-7.05 (m, 3H), 6.77 (s, 4H), 6.73 (dd, J=3.2, 1.6 Hz, 1H), 5.82 (t, J=7.6 Hz, 1H), 4.01-3.91 (m, 2H), 3.70-3.62 (m, 1H), 3.62-3.57 (m, 2H), 3.56-3.51 (m, 2H), 3.46 (d, J=7.6 Hz, 2H), 3.30 (s, 1H), 3.27 (s, 3H), 2.95-2.87 (m, 1H), 2.85-2.70 (m, 2H), 2.45-2.35 (m, 1H). MS: M/e 608 (M+1)⁺.

### Compound A20: 2-(5-amino-2-(furan-2-yl)-7H-pyrazolo[4,3-e][1,2,4]triazolo[1,5-c]pyrimidin-7-yl)-1-(4-(2,4-difluorophenyl)piperazin-1-yl)pentan-1-one

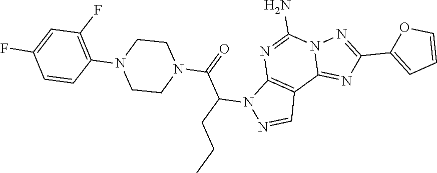 [SMILES: CCCC(C(=O)N1CCN(c2ccc(F)cc2F)CC1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)pentanoic acid (30 mg, 0.09 mmol), 1-(2,4-difluorophenyl)piperazine (19 mg, 0.09 mmol), HATU (41 mg, 0.1 mmol) and DIEA (23 mg, 0.18 mmol) in DMF (5 mL) was stirred at rt for 3 hrs. The solution was added with water (5 mL), extracted with ethyl acetate (10 mL) and washed with brine (10 mL). The organic layer was dried, concentrated and purified by column chromatography (PE:EA=1:1) to get the desired product (26 mg, 56%). ¹H NMR (400 MHz, DMSO-d6) δ 8.21 (s, 1H), 8.15 (br.s, 2H), 7.95 (s, 1H), 7.23-7.14 (m, 2H), 6.96-6.93 (m, 2H), 6.74 (s, 1H), 5.60-5.57 (m, 1H), 3.72-3.51 (m, 4H), 2.93-2.69 (m, 3H), 2.40 (br.s, 1H), 2.18-1.99 (m, 2H), 1.23-1.11 (m, 2H), 0.89-0.86 (m, 3H) ppm. MS: M/e 522 (M+1)⁺

### Compound A21: 2-(5-amino-2-(furan-2-yl)-7H-pyrazolo[4,3-e][1,2,4]triazolo[1,5-c]pyrimidin-7-yl)-2-cyclopropyl-1-(4-(4-(2-(2-methoxyethoxy)ethoxy)phenyl)piperazin-1-yl)ethan-1-one

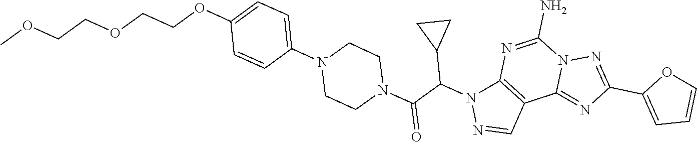 [SMILES: COCCOCCOc1ccc(N2CCN(C(=O)C(C3CC3)n3ncc4c3nc(N)n3nc(-c5ccco5)nc43)CC2)cc1]

A mixture of 2-(5-amino-2-(furan-2-yl)-7H-pyrazolo[4,3-e][1,2,4]triazolo[1,5-c]pyrimidin-7-yl)-2-cyclopropylacetic acid (50 mg, 0.15 mmol), 1-(4-(2-(2-methoxyethoxy)ethoxy)phenyl)piperazine (50 mg, 0.18 mmol), HATU (84 mg, 0.22 mmol) and TEA (30 mg, 0.30 mmol) in DMF (10 mL) was stirred for 4 hours at RT. The reaction mixture was poured into H₂O (20 mL) and extracted with EtOAc (50 mL×3). The combined organic layers were washed with brine, dried over Na₂SO₄, concentrated and purified by column chromatography (petroleum ether/EtOAc=1:1˜100% EtOAc) to give the target compound (21 mg, 23.3%). ¹H NMR (400 MHz, CD₃OD) δ 8.16 (s, 1H), 7.75 (s, 1H), 7.25 (d, J=3.4 Hz, 1H), 6.85-6.75 (m, 4H), 6.69-6.62 (m, 1H), 5.00 (d, J=9.7 Hz, 1H), 4.06-3.96 (m, 2H), 3.94-3.83 (m, 1H), 3.79-3.73 (m, 2H), 3.71-3.56 (m, 4H), 3.55-3.45 (m, 3H), 3.33 (s, 3H), 3.06 (s, 1H), 2.90 (s, 2H), 2.40 (s, 1H), 1.88 (t, J=8.9 Hz, 1H), 0.78 (t, J=8.5 Hz, 1H), 0.57-0.46 (m, 3H). MS: M/e 602 (M+1)⁺.

### Compound A22: 2-(5-amino-2-(furan-2-yl)-7H-pyrazolo[4,3-e][1,2,4]triazolo[1,5-c]pyrimidin-7-yl)-1-(4-(2,4-difluorophenyl)piperazin-1-yl)-4-methylpentan-1-one

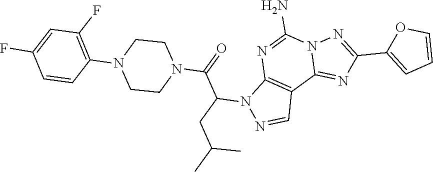 [SMILES: CC(C)CC(C(=O)N1CCN(c2ccc(F)cc2F)CC1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)-4-methylpentanoic acid (35.5 0.1 mmol), 1-(2,4-difluorophenyl)piperazine (19.8 mg, 0.1 mmol), HATU (46 mg, 0.12 mmol) and DIPEA (25.8 mg, 0.2 mmol) in DMF (3 mL) was stirred overnight. The reaction mixture was poured into H₂O (15 mL) and extracted with EtOAc (20 mL×3). The combined organic layers were washed with brine, dried over Na₂SO₄, concentrated and purified by column chromatography (petroleum ether/EtOAc=10:1˜3:1) to give the target compound (40 mg, 74.6%). ¹H NMR (400 MHz, DMSO-d6) δ 8.21 (s, 1H), 8.15 (s, 2H), 7.95 (m, 1H), 7.23 (d, J=3.2 Hz, 1H), 7.21-7.12 (m, 1H), 7.02-6.91 (m, 2H), 6.74 (m, 1H), 5.64 (dd, J=9.6, 4.4 Hz, 1H), 3.74 (m, 1H), 3.62 (m, 2H), 3.49 (m, 1H), 2.94 (m, 2H), 2.75 (m, 1H), 2.39 (m, 1H), 2.31-2.19 (m, 1H), 1.91-1.80 (m, 1H), 1.27 (mz, 1H), 0.95 (d, J=6.4 Hz, 3H), 0.83 (d, J=6.4 Hz, 3H) ppm. MS: M/e 536 (M+1)⁺.

Compound A22 was separated into two enantiomeric stereoisomers (Compound A22a, earlier peak, and Compound A22b, later peak) by chiral prep-HPLC. The chiral separation conditions are shown below.

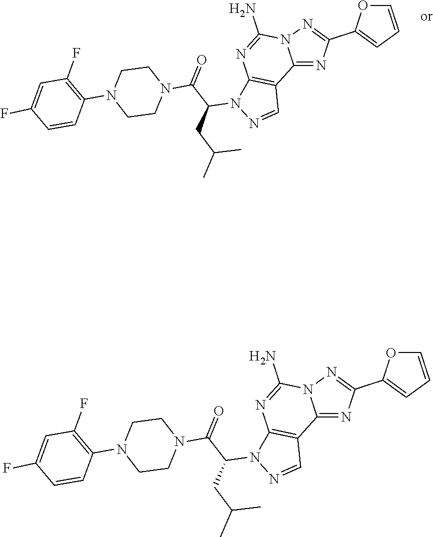 [SMILES: CC(C)C[C@@H](C(=O)N1CCN(c2ccc(F)cc2F)CC1)n1ncc2c1nc(N)n1nc(-c3ccco3)nc21 ; CC(C)C[C@H](C(=O)N1CCN(c2ccc(F)cc2F)CC1)n1ncc2c1nc(N)n1nc(-c3ccco3)nc21]

| Column | CHIRALPAK IG |
|---|---|
| Column size | 2 cm × 25 cm, 5 um |
| Injection | 0.8 mL |
| Mobile phase | Hex:EtOH = 50:50 |
| Flow rate | 18 ml/min |
| Wavelength | UV 220 nm |
| Temperature | 25° C. |
| Sample solution | 42.5 mg/ml in DCM:EtOH = 1:1 |
| Prep-HPLC equipment | Prep-Gilson-HPLC |

### Compound A23: 2-(5-amino-2-(furan-2-yl)-7H-pyrazolo[4,3-e][1,2,4]triazolo[1,5-c]pyrimidin-7-yl)-1-(4-(2-fluoro-4-(2-methoxyethoxy)phenyl)piperazin-1-yl)-4-methylpentan-1-one

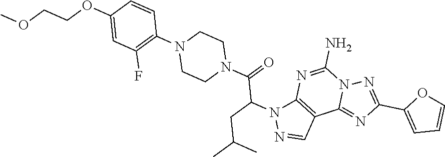 [SMILES: COCCOc1ccc(N2CCN(C(=O)C(CC(C)C)n3ncc4c3nc(N)n3nc(-c5ccco5)nc43)CC2)c(F)c1]

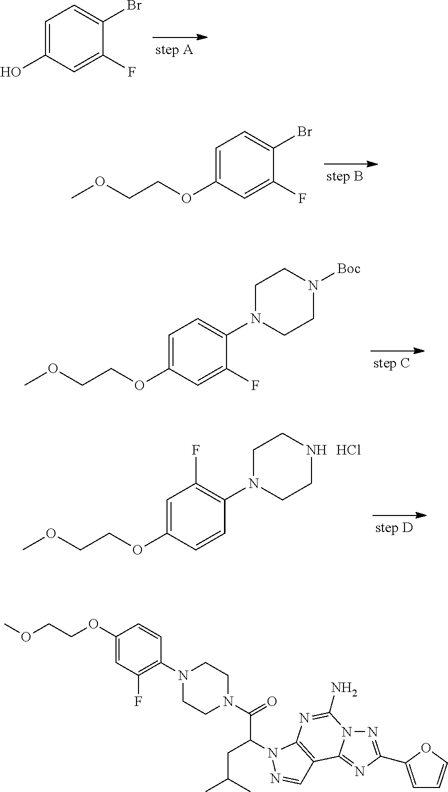 [SMILES: Oc1ccc(Br)c(F)c1 ; COCCOc1ccc(Br)c(F)c1 ; COCCOc1ccc(N2CCN(C(=O)OC(C)(C)C)CC2)c(F)c1 ; COCCOc1ccc(N2CCNCC2)c(F)c1 ; Cl ; COCCOc1ccc(N2CCN(C(=O)C(CC(C)C)n3ncc4c3nc(N)n3nc(-c5ccco5)nc43)CC2)c(F)c1]

### Step A: 1-bromo-2-fluoro-4-(2-methoxyethoxy)benzene

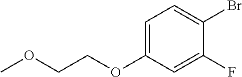

A mixture of 4-bromo-3-fluorophenol (5 g, 26.1 mmol), 1-bromo-2-methoxyethane (4 g, 28.7 mmol) and K₂CO₃ (7.2 g, 52.2 mmol) in CH₃CN (50 mL) was stirred at 60° C. overnight. The reaction mixture was poured into H₂O (200 mL) and extracted with EtOAc (100 mL×3). The combined organic layers were washed with brine, dried over Na₂SO₄, concentrated under high vacuum to give the target compound (5.7 g, 87.3%) as yellow oil. MS: M/e 249/251 (M+1)⁺.

### Step B: tert-butyl 4-(2-fluoro-4-(2-methoxyethoxy)phenyl)piperazine-1-carboxylate

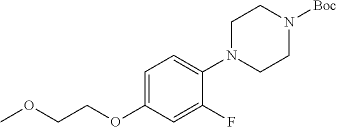 [SMILES: COCCOc1ccc(N2CCN(C(=O)OC(C)(C)C)CC2)c(F)c1]

A mixture of the product of step A (3.5 g, 14 mm), tert-butyl piperazine-1-carboxylate (2.6 g, 14 mmol), Pd(dba)₃ (1.28 g, 1.4 mmol), X-phos (1.3 g, 2.8 mmol) and Cs₂CO₃ (9.2 g, 28 mmol) in toluene (100 mL) was stirred at 120° C. for 5 hours. Most toluene was removed to give the residue, treated with EtOAc/H₂O (100 mL/50 mL). The organic layer was separated, washed with brine, dried over Na₂SO₄, concentrated and purified by column chromatography (petroleum ether/EtOAc=20:1˜10:1) to give the target compound (2.5 g, 50.4%) as yellow oil. MS: M/e 355 (M+1)⁺.

### Step C: 1-(2-fluoro-4-(2-methoxyethoxy)phenyl)piperazine Hydrochloride

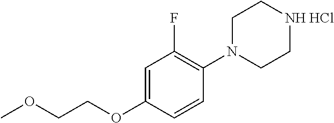

To a stirred mixture of the product of step B (1.25 g, 3.53 mmol) in CH₂Cl₂ (10 mL) was added EtOAc/HCl (g) (4.0 M, 5 mL). After the addition, the reaction mixture was stirred for a weekend. The reaction mixture was filtered and the cake was collected, dried to give the target compound (500 mg, 48.8%) as a tan solid. MS: M/e 255 (M+1)⁺.

### Step D: 2-(5-amino-2-(furan-2-yl)-7H-pyrazolo[4,3-e][1,2,4]triazolo[1,5-c]pyrimidin-7-yl)-1-(4-(2-fluoro-4-(2-methoxyethoxy)phenyl)piperazin-1-yl)-4-methylpentan-1-one

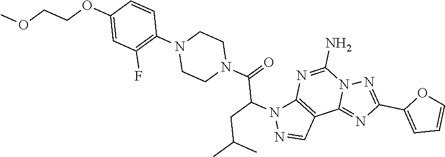 [SMILES: COCCOc1ccc(N2CCN(C(=O)C(CC(C)C)n3ncc4c3nc(N)n3nc(-c5ccco5)nc43)CC2)c(F)c1]

A mixture of 2-(5-amino-2-(furan-2-yl)-7H-pyrazolo[4,3-e][1,2,4]triazolo[1,5-c]pyrimidin-7-yl)-4-methylpentanoic acid (35.5 mg, 0.1 mmol), the product of step C (29 mg, 0.1 mmol), HATU (46 mg, 0.12 mmol) and DIPEA (38.7 mg, 0.3 mmol) in DMF (3 mL) was stirred for 3 hours. The reaction mixture was poured into H₂O (15 mL) and extracted with EtOAc (15 mL×3). The combined organic layers were washed with brine, dried over Na₂SO₄, concentrated and purified by column chromatography (petroleum ether/EtOAc=2:1˜100% EtOAc) to give the target compound (40 mg, 76.6%). ¹H NMR (400 MHz, DMSO-d6) δ 8.21 (s, 1H), 8.16 (s, 2H), 7.95 (s, 1H), 7.23 (d, J=2.8 Hz, 1H), 6.89-6.64 (m, 4H), 5.64 (m, 1H), 4.01 (m, 2H), 3.71 (m, 1H), 3.60 (m, 4H), 3.47 (m, 1H), 3.27 (s, 3H), 2.88 (m, 2H), 2.69 (m, 1H), 2.37-2.20 (m, 2H), 1.86-1.84 (m, 1H), 1.32-1.24 (m, 1H), 0.94 (d, J=6.4 Hz, 3H), 0.83 (d, J=6.4 Hz, 3H) ppm. MS: M/e 592 (M+1)⁺.

### Compound A24: 2-(5-amino-2-(furan-2-yl)-7H-pyrazolo[4,3-e][1,2,4]triazolo[1,5-c]pyrimidin-7-yl)-1-(4-(2-fluoro-4-(2-methoxyethoxy)phenyl)piperazin-1-yl)pentan-1-one

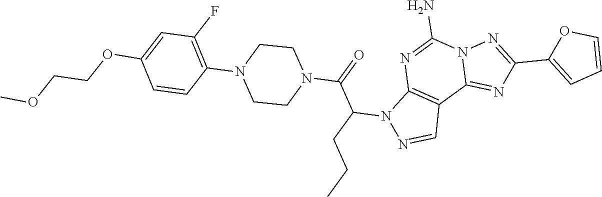 [SMILES: CCCC(C(=O)N1CCN(c2ccc(OCCOC)cc2F)CC1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)pentanoic acid (30 mg, 0.09 mmol), 1-(2-fluoro-4-(2-methoxyethoxy)phenyl)piperazine (26 mg, 0.09 mmol), HATU (41 mg, 0.1 mmol) and DIEA (23 mg, 0.18 mmol) in DMF (5 mL) was stirred at rt for 2 hrs. The solution was added with water (5 mL), extracted with ethyl acetate (10 mL) and washed with brine (10 mL). The organic layer was dried, concentrated and purified by column chromatography (PE:EA=1:1) to get the desired product (23 mg, 43%). ¹H NMR (400 MHz, DMSO-d6) δ 8.21 (s, 1H), 8.14 (br.s, 2H), 7.95 (s, 1H), 7.23 (d, J=4.0 Hz, 1H), 6.74-6.64 (m, 4H), 5.60-5.56 (m, 1H), 4.04-4.00 (m, 2H), 3.60-3.53 (m, 6H), 3.27 (s, 3H), 2.89-2.67 (m, 3H), 2.18-1.99 (m, 3H), 1.23-1.11 (m, 2H), 0.94-0.86 (m, 3H) ppm. MS: M/e 578 (M+1)⁺

Compound A24 was separated into two enantiomeric stereoisomers (Compound A24a, earlier peak, and Compound A24b, later peak) by chiral prep-HPLC. The chiral separation conditions are shown below.

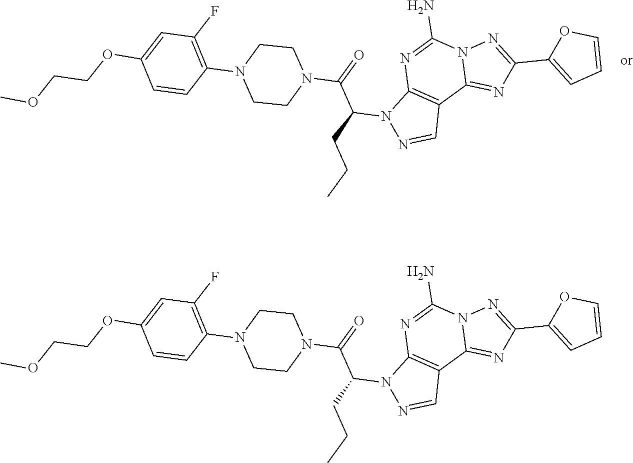 [SMILES: CCC[C@@H](C(=O)N1CCN(c2ccc(OCCOC)cc2F)CC1)n1ncc2c1nc(N)n1nc(-c3ccco3)nc21 ; CCC[C@H](C(=O)N1CCN(c2ccc(OCCOC)cc2F)CC1)n1ncc2c1nc(N)n1nc(-c3ccco3)nc21]

| Column | CHIRALPAK Cellulose-IC |
|---|---|
| Column size | 2 cm × 25 cm, 5 um |
| Injection | 2 mL |
| Mobile phase | (Hex:DCM = 5:1):EtOH = 70:30 |
| Flow rate | 20 ml/min |
| Wavelength | UV 220 nm |
| Temperature | 25° C. |
| Sample solution | 34.3 mg/ml in DCM:EtOH = 1:1 |
| Prep-HPLC equipment | Prep-YMC-HPLC |

### Compound A25: 2-(5-amino-2-(furan-2-yl)-7H-pyrazolo[4,3-e][1,2,4]triazolo[1,5-c]pyrimidin-7-yl)-1-(4-(2,6-difluoro-4-(2-methoxyethoxy)phenyl)piperazin-1-yl)pentan-1-one

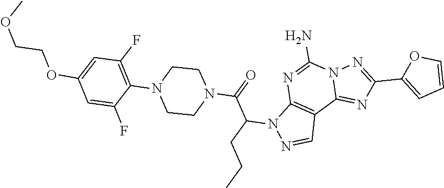 [SMILES: CCCC(C(=O)N1CCN(c2c(F)cc(OCCOC)cc2F)CC1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)pentanoic acid (40 mg, 0.12 mmol), 1-(2,6-difluoro-4-(2-methoxyethoxy)phenyl)piperazine trifluoroacetic acid salt (51 mg, 0.13 mmol), HATU (55 mg, 0.14 mmol) and DIEA (36 mg, 0.28 mmol) in DMF (10 mL) was stirred at rt for 2 hrs. The solution was added with water (10 mL), extracted with ethyl acetate (10 mL) and washed with brine (10 mL). The organic layer was dried, concentrated and purified by column chromatography (PE:EA=1:1) to get the desired product (45 mg, 64%). ¹H NMR (400 MHz, DMSO-d6) δ 8.23 (s, 1H), 8.16 (br.s, 2H), 7.95 (s, 1H), 7.24 (d, J=4.0 Hz, 1H), 6.75-6.65 (m, 3H), 5.59-5.56 (m, 1H), 4.03-4.00 (m, 2H), 3.63-3.50 (m, 6H), 3.26 (s, 3H), 2.85-2.69 (m, 3H), 2.39-1.99 (m, 3H), 1.12-1.05 (m, 2H), 0.93-0.86 (m, 3H) ppm. MS: M/e 596 (M+1)⁺

### Compound A26: 2-(5-amino-2-(furan-2-yl)-7H-pyrazolo[4,3-e][1,2,4]triazolo[1,5-c]pyrimidin-7-yl)-3-methoxy-1-(4-(4-(2-methoxyethoxy)phenyl)piperazin-1-yl)propan-1-one

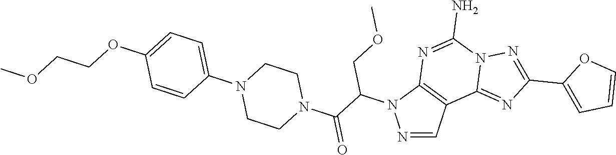 [SMILES: COCCOc1ccc(N2CCN(C(=O)C(COC)n3ncc4c3nc(N)n3nc(-c5ccco5)nc43)CC2)cc1]

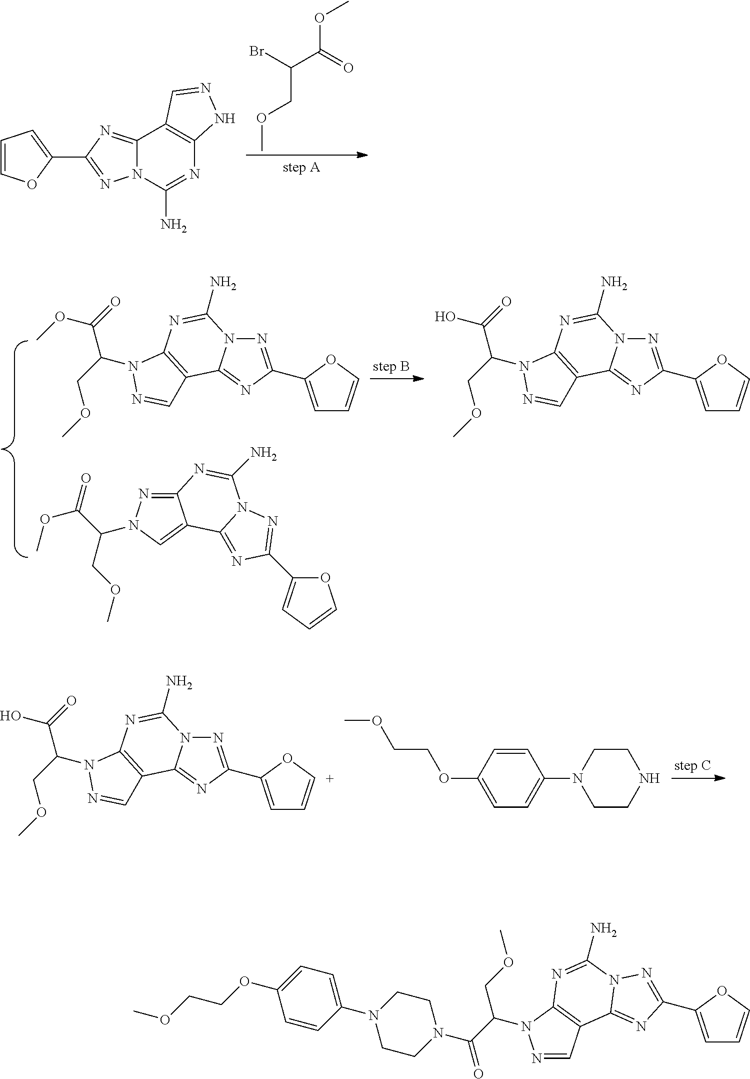

### Step A: methyl 2-(5-amino-2-(furan-2-yl)-7H-pyrazolo[4,3-e][1,2,4]triazolo[1,5-c]pyrimidin-7-yl)-3-methoxypropanoate

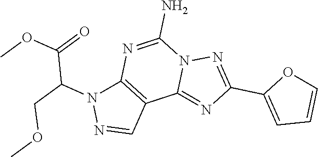 [SMILES: COCC(C(=O)OC)n1ncc2c1nc(N)n1nc(-c3ccco3)nc21]

To a stirred solution of 2-(furan-2-yl)-7H-pyrazolo[4,3-e][1,2,4]triazolo[1,5-c]pyrimidin-5-amine (1.0 g, 4.2 mmol) in DMF (50 mL) was added K₂CO₃ (1.3 g, 9.4 mmol) and 2-bromo-1,3-dimethoxypropan-1-one (1.0 g, 5.5 mmol). After the addition, the reaction mixture was stirred overnight at 60° C. The reaction mixture was poured into H₂O (50 mL) and extracted with EtOAc (100 mL×3). The combined organic layers were washed with brine, dried over Na₂SO₄, concentrated and purified by column chromatography (petroleum ether/EtOAc=2:1˜1:2) to give methyl 2-(5-amino-2-(furan-2-yl)-7H-pyrazolo[4,3-e][1,2,4]triazolo[1,5-c]pyrimidin-7-yl)-3-methoxypropanoate (300 mg, 41.5%) and methyl 2-(5-amino-2-(furan-2-yl)-8H-pyrazolo[4,3-e][1,2,4]triazolo[1,5-c]pyrimidin-8-yl)-3-methoxypropanoate (80 mg, 11.1%) as white solids. MS: M/e 358 (M+1)⁺.

### Step B: 2-(5-amino-2-(furan-2-yl)-7H-pyrazolo[4,3-e][1,2,4]triazolo[1,5-c]pyrimidin-7-yl)-3-methoxypropanoic Acid

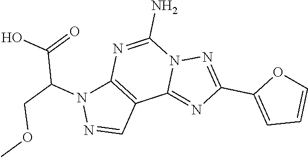 [SMILES: COCC(C(=O)O)n1ncc2c1nc(N)n1nc(-c3ccco3)nc21]

To a stirred mixture of methyl 2-(5-amino-2-(furan-2-yl)-7H-pyrazolo[4,3-e][1,2,4]triazolo[1,5-c]pyrimidin-7-yl)-3-methoxypropanoate (400 mg, 1.2 mmol) in MeOH/H₂O (9 mL/2 mL) was added aq.NaOH (4.0 M, 2 mL). After the addition, the reaction mixture was stirred for 3 hours at RT. Most of solvent was removed to give the aqueous layer, then acidified to pH=3˜4 with aq.HCl and filtered, the cake was collected, dried to give the target compound (140 mg, 36.3%) as a white solid. MS: M/e 344 (M+1)⁺.

### Step C: 2-(5-amino-2-(furan-2-yl)-7H-pyrazolo[4,3-e][1,2,4]triazolo[1,5-c]pyrimidin-7-yl)-3-methoxy-1-(4-(4-(2-methoxyethoxy)phenyl)piperazin-1-yl)propan-1-one

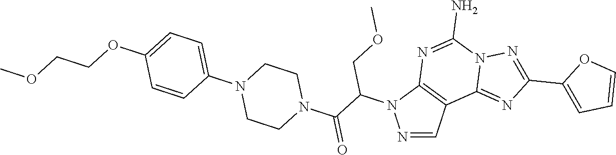 [SMILES: COCCOc1ccc(N2CCN(C(=O)C(COC)n3ncc4c3nc(N)n3nc(-c5ccco5)nc43)CC2)cc1]

A mixture of the product of step B (50 mg, 0.15 mmol), 1-(4-(2-methoxyethoxy)phenyl)piperazine (40 mg, 0.18 mmol), HATU (84 mg, 0.22 mmol) and TEA (30 mg, 0.30 mmol) in DMF (10 mL) was stirred overnight at RT. The reaction mixture was poured into H₂O (20 mL) and extracted with EtOAc (50 mL×3). The combined organic layers were washed with brine, dried over Na₂SO₄, concentrated and purified by column chromatography (petroleum ether/EtOAc=1:1˜100% EtOAc) to give the target compound (11 mg, 13.4%). ¹H NMR (400 MHz, DMSO-d6) δ 8.22 (br.s, 3H), 7.95 (d, J=0.9 Hz, 1H), 7.24 (d, J=2.9 Hz, 1H), 6.82-6.72 (m, 6H), 5.79 (dd, J=8.4, 5.3 Hz, 1H), 4.06-3.94 (m, 4H), 3.76-3.69 (m, 1H), 3.61-3.56 (m, 2H), 3.53-3.47 (m, 2H), 3.27 (s, 3H), 3.21 (s, 3H), 3.08-3.01 (m, 1H), 2.98-2.92 (m, 1H), 2.80-2.73 (m, 1H), 2.30-2.23 (m, 1H) ppm. MS: M/e 562 (M+1)⁺.

### Compound A27: 2-(5-amino-2-(furan-2-yl)-7H-pyrazolo[4,3-e][1,2,4]triazolo[1,5-c]pyrimidin-7-yl)-3-methoxy-1-(4-(4-(2-(2-methoxyethoxy)ethoxy)phenyl)piperazin-1-yl)propan-1-one

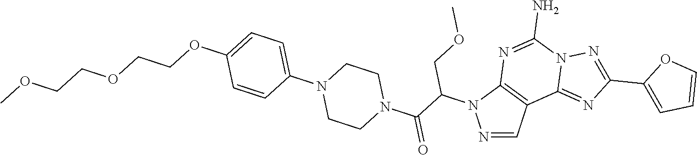 [SMILES: COCCOCCOc1ccc(N2CCN(C(=O)C(COC)n3ncc4c3nc(N)n3nc(-c5ccco5)nc43)CC2)cc1]

A mixture of 2-(5-amino-2-(furan-2-yl)-7H-pyrazolo[4,3-e][1,2,4]triazolo[1,5-c]pyrimidin-7-yl)-3-methoxypropanoic acid (50 mg, 0.15 mmol), 1-(4-(2-(2-methoxyethoxy)ethoxy)phenyl)piperazine (45 mg, 0.19 mmol), HATU (84 mg, 0.22 mmol) and TEA (30 mg, 0.30 mmol) in DMF (10 mL) was stirred for 16 hours at RT. The reaction mixture was poured into H₂O (20 mL) and extracted with EtOAc (50 mL×3). The combined organic layers were washed with brine, dried over Na₂SO₄, concentrated and purified by column chromatography (petroleum ether/EtOAc=1:1˜100% EtOAc) to give the target compound (19 mg, 20.9%). ¹H NMR (400 MHz, DMSO-d6) δ 8.22 (br.s, 3H), 7.95 (s, 1H), 7.24 (d, J=2.9 Hz, 1H), 6.82-6.72 (m, 6H), 5.79 (dd, J=8.4, 5.3 Hz, 1H), 4.07-3.94 (m, 4H), 3.69-3.64 (m, 3H), 3.57-3.53 (m, 4H), 3.43 (dd, J=5.8, 3.7 Hz, 3H), 3.22 (s, 3H), 3.20 (s, 3H), 3.07-3.00 (m, 1H), 2.97-2.91 (m, 1H), 2.81-2.72 (m, 1H), 2.30-2.23 (m, 1H) ppm. MS: M/e 606 (M+1)⁺.

### Compound A28: 2-(5-amino-2-(furan-2-yl)-7H-pyrazolo[4,3-e][1,2,4]triazolo[1,5-c]pyrimidin-7-yl)-2-(4-fluorophenyl)-1-(4-(4-(2-methoxyethoxy)phenyl)piperazin-1-yl)ethan-1-one

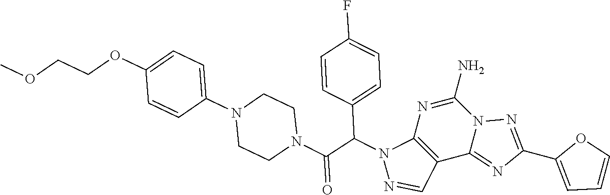 [SMILES: COCCOc1ccc(N2CCN(C(=O)C(c3ccc(F)cc3)n3ncc4c3nc(N)n3nc(-c5ccco5)nc43)CC2)cc1]

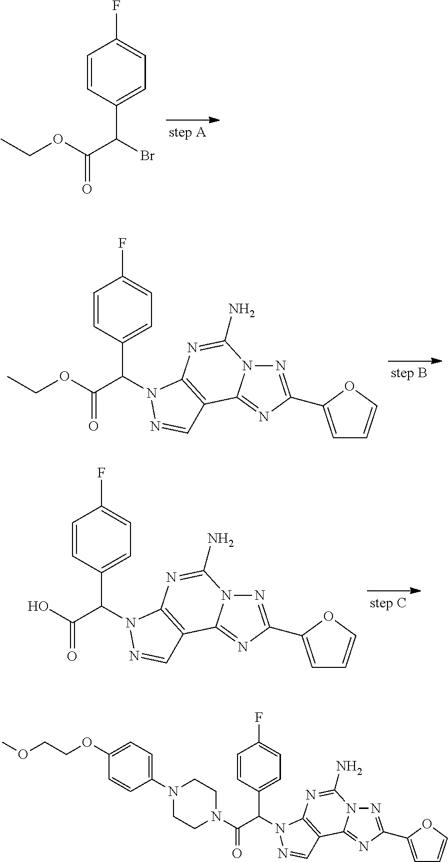 [SMILES: CCOC(=O)C(Br)c1ccc(F)cc1 ; CCOC(=O)C(c1ccc(F)cc1)n1ncc2c1nc(N)n1nc(-c3ccco3)nc21 ; Nc1nc2c(cnn2C(C(=O)O)c2ccc(F)cc2)c2nc(-c3ccco3)nn12 ; COCCOc1ccc(N2CCN(C(=O)C(c3ccc(F)cc3)n3ncc4c3nc(N)n3nc(-c5ccco5)nc43)CC2)cc1]

### Step A: ethyl 2-(5-amino-2-(furan-2-yl)-7H-pyrazolo[4,3-e][1,2,4]triazolo[1,5-c]pyrimidin-7-yl)-2-(4-fluorophenyl)acetate

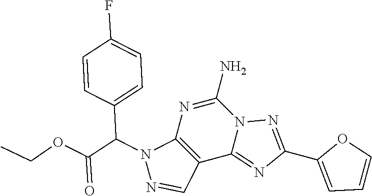 [SMILES: CCOC(=O)C(c1ccc(F)cc1)n1ncc2c1nc(N)n1nc(-c3ccco3)nc21]

To a stirred solution of 2-(furan-2-yl)-7H-pyrazolo[4,3-e][1,2,4]triazolo[1,5-c]pyrimidin-5-amine (964 mg, 4 mmol) in DMF (20 mL) was added K₂CO₃ (1.1 g, 8 mmol), then a solution of ethyl 2-bromo-2-(4-fluorophenyl)acetate (1 g, 4 mmol) in DMF (2 mL) was added dropwise. After the addition, the reaction mixture was stirred overnight. The reaction mixture was poured into H₂O (50 mL) and extracted with EtOAc (50 mL×4). The combined organic layers were washed with brine, dried over Na₂SO₄, concentrated to give the residue, which was purified by column chromatography (petroleum ether/EtOAc=2:1˜100% EtOAc) to give the target compound (300 mg, 17.8%) as a white solid. MS: M/e 422 (M+1)⁺.

### Step B: 2-(5-amino-2-(furan-2-yl)-7H-pyrazolo[4,3-e][1,2,4]triazolo[1,5-c]pyrimidin-7-yl)-2-(4-fluorophenyl)acetic Acid

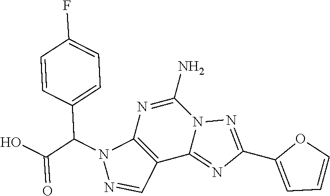 [SMILES: Nc1nc2c(cnn2C(C(=O)O)c2ccc(F)cc2)c2nc(-c3ccco3)nn12]

To a stirred solution of the product of step A (300 mg, 0.71 mmol) in MeOH (10 mL) was added aq.NaOH (4 mL, 2.0 M). After the addition, the reaction mixture was stirred for 3 hours. Most of the MeOH was removed to give the aqueous layer, which was acidified to pH=3˜4 with aq.HCl and filtered. The cake was collected, dried to give the target compound (172 mg, 61.6%) as a white solid. MS: M/e 394 (M+1)⁺.

### Step C: 2-(5-amino-2-(furan-2-yl)-7H-pyrazolo[4,3-e][1,2,4]triazolo[1,5-c]pyrimidin-7-yl)-2-(4-fluorophenyl)-1-(4-(4-(2-methoxyethoxy)phenyl)piperazin-1-yl)ethan-1-one

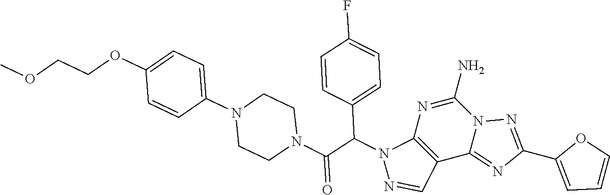 [SMILES: COCCOc1ccc(N2CCN(C(=O)C(c3ccc(F)cc3)n3ncc4c3nc(N)n3nc(-c5ccco5)nc43)CC2)cc1]

A mixture of the product of step B (30 mg, 0.076 mmol), 1-(4-(2-methoxyethoxy)phenyl)piperazine (18 mg, 0.076 mmol), HATU (34.4 mg, 0.09 mmol) and DIPEA (19.6 mg, 0.152 mmol) in DMF (3 mL) was stirred for 2 hours. The reaction mixture was poured into H₂O (15 mL), and extracted with EtOAc (20 mL×2). The combined organic layers were washed with brine, dried over Na₂SO₄, concentrated and purified by column chromatography (petroleum ether/EtOAc=1:1˜100% EtOAc) to give the target compound, which was further purified by prep-TLC (EtOAc) to give the target compound (5 mg, 10.7%). ¹H NMR (400 MHz, DMSO-d6) δ 8.22 (s, 1H), 8.17 (s, 2H), 7.95 (s, 1H), 7.51-7.40 (m, 2H), 7.27-7.15 (m, 3H), 6.90 (s, 1H), 6.80 (m, 4H), 6.74 (s, 1H), 3.97 (m, 2H), 3.73-3.55 (m, 4H), 3.38-3.32 (m, 2H), 3.28 (s, 3H), 2.96 (m, 2H), 2.64 (m, 2H) ppm. MS: M/e 612 (M+1)⁺.

### Compound A29: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(4-methoxyphenyl ethan-1-one

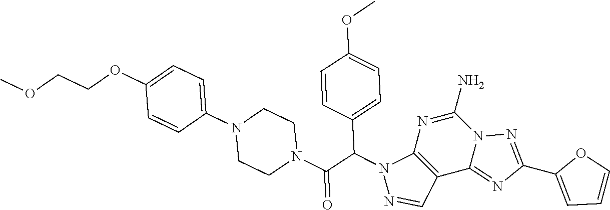 [SMILES: COCCOc1ccc(N2CCN(C(=O)C(c3ccc(OC)cc3)n3ncc4c3nc(N)n3nc(-c5ccco5)nc43)CC2)cc1]

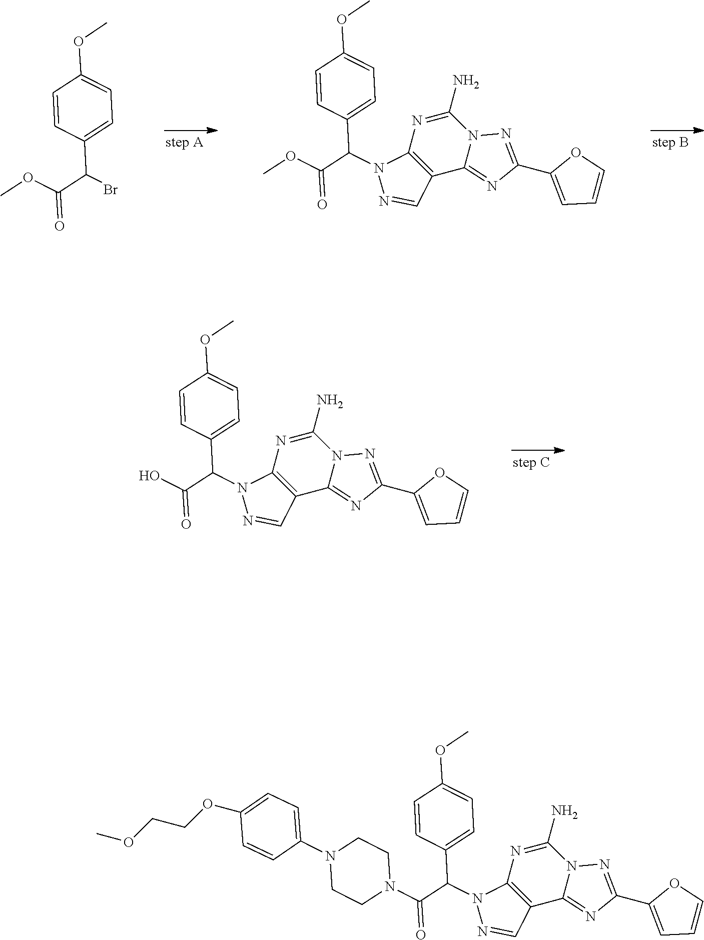 [SMILES: COC(=O)C(Br)c1ccc(OC)cc1 ; COC(=O)C(c1ccc(OC)cc1)n1ncc2c1nc(N)n1nc(-c3ccco3)nc21 ; COc1ccc(C(C(=O)O)n2ncc3c2nc(N)n2nc(-c4ccco4)nc32)cc1 ; COCCOc1ccc(N2CCN(C(=O)C(c3ccc(OC)cc3)n3ncc4c3nc(N)n3nc(-c5ccco5)nc43)CC2)cc1]

### Step A: methyl 2-(5-amino-2-(furan-2-yl)-7H-pyrazolo[4,3-e][1,2,4]triazolo[1,5-c]pyrimidin-7-yl)-2-(4-methoxyphenyl)acetate

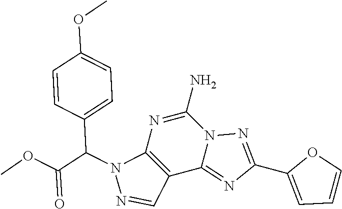 [SMILES: COC(=O)C(c1ccc(OC)cc1)n1ncc2c1nc(N)n1nc(-c3ccco3)nc21]

To a stirred solution of 2-(furan-2-yl)-7H-pyrazolo[4,3-e][1,2,4]triazolo[1,5-c]pyrimidin-5-amine (482 mg, 2 mmol) in DMF (20 mL) was added K₂CO₃ (552 mg, 4 mmol), then a solution of methyl 2-bromo-2-(4-methoxyphenyl)acetate (518 mg, 2 mmol) in DMF (2 mL) was added dropwise. After the addition, the reaction mixture was stirred overnight. The reaction mixture was poured into H₂O (50 mL) and extracted with EtOAc (30 mL×3). The combined organic layers were washed with brine, dried over Na₂SO₄, concentrated to give the residue, which was purified by column chromatography (petroleum ether/EtOAc=2:1˜100% EtOAc) to give the target compound (100 mg, 11.9%) as a white solid. MS: M/e 420 (M+1)⁺.

### Step B: 2-(5-amino-2-(furan-2-yl)-7H-pyrazolo[4,3-e][1,2,4]triazolo[1,5-c]pyrimidin-7-yl)-2-(4-methoxyphenyl)acetic Acid

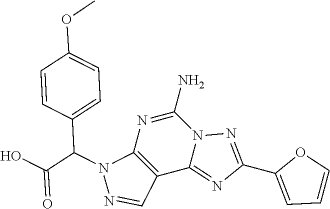 [SMILES: COc1ccc(C(C(=O)O)n2ncc3c2nc(N)n2nc(-c4ccco4)nc32)cc1]

To a stirred solution of the product of step A (100 mg, 0.238 mmol) in MeOH (5 mL) was added aq.NaOH (3 mL, 2.0 M). After the addition, the reaction mixture was stirred for 3 hours. Most of the MeOH was removed to give the aqueous layer, which was acidified to pH=3˜4 with aq.HCl and filtered. The cake was collected, dried to give the target compound (68 mg, 70.5%) as a white solid. MS: M/e 406 (M+1)⁺.

### Step C: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(4-methoxyphenyl)ethan-1-one

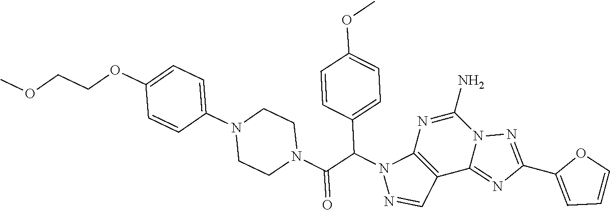 [SMILES: COCCOc1ccc(N2CCN(C(=O)C(c3ccc(OC)cc3)n3ncc4c3nc(N)n3nc(-c5ccco5)nc43)CC2)cc1]

A mixture of the product of step B (30 mg, 0.074 mmol), 1-(4-(2-methoxyethoxy)phenyl)piperazine (17.48 mg, 0.074 mmol), HATU (34.4 mg, 0.09 mmol) and DIPEA (19.6 mg, 0.152 mmol) in DMF (3 mL) was stirred for 2 hours. The reaction mixture was poured into H₂O (15 mL), and extracted with EtOAc (20 mL×2). The combined organic layers were washed with brine, dried over Na₂SO₄, concentrated and purified by column chromatography (petroleum ether/EtOAc=1:1˜100% EtOAc) to give the target compound (20 mg, 43.4%). ¹H NMR (400 MHz, DMSO-d6) δ 8.16 (s, 1H), 8.14 (s, 2H), 7.95 (s, 1H), 7.38-7.35 (d, J=8.4 Hz, 2H), 7.24 (d, J=3.2 Hz, 1H), 6.94-6.92 (d, J=8.8 Hz, 2H), 6.86-6.76 (m, 4H), 6.74 (m, 1H), 4.01-3.95 (m, 2H), 3.75 (s, 3H), 3.68 (m, 1H), 3.63-3.54 (m, 3H), 3.28 (s, 3H), 3.0-2.90 (m, 2H), 2.77-2.54 (m, 2H) ppm. MS: M/e 624 (M+1)⁺.

### Compound A30: 2-(5-amino-2-(furan-2-yl)-7H-pyrazolo[4,3-e][1,2,4]triazolo[1,5-c]pyrimidin-7-yl)-1-(4-(4-(2-(methylamino)ethoxy)phenyl)piperazin-1-yl)propan-1-one

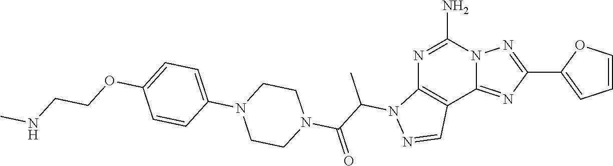 [SMILES: CNCCOc1ccc(N2CCN(C(=O)C(C)n3ncc4c3nc(N)n3nc(-c5ccco5)nc43)CC2)cc1]

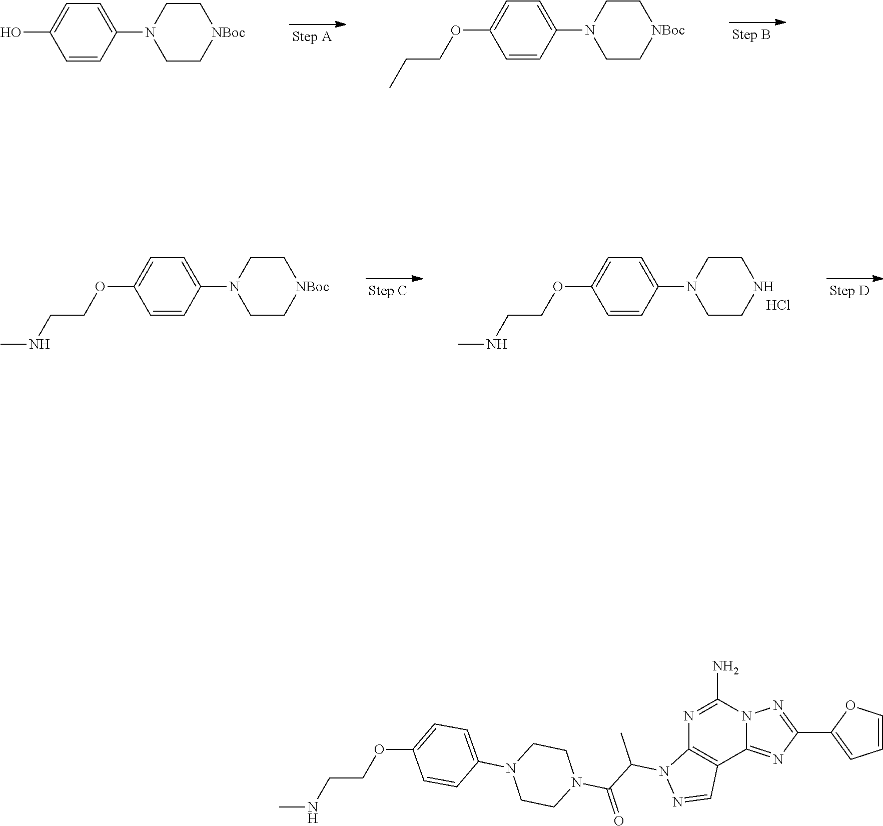 [SMILES: CC(C)(C)OC(=O)N1CCN(c2ccc(O)cc2)CC1 ; CCCOc1ccc(N2CCN(C(=O)OC(C)(C)C)CC2)cc1 ; CNCCOc1ccc(N2CCN(C(=O)OC(C)(C)C)CC2)cc1 ; CNCCOc1ccc(N2CCNCC2)cc1 ; Cl ; CNCCOc1ccc(N2CCN(C(=O)C(C)n3ncc4c3nc(N)n3nc(-c5ccco5)nc43)CC2)cc1]

### Step A: tert-butyl 4-(4-(2-bromoethoxy)phenyl)piperazine-1-carboxylate

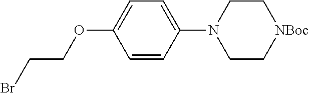 [SMILES: CC(C)(C)OC(=O)N1CCN(c2ccc(OCCBr)cc2)CC1]

To a stirred solution of tert-butyl 4-(4-hydroxyphenyl)piperazine-1-carboxylate (7 g, 25.2 mmol) and K₂CO₃ (7 g, 52.9 mmol) in Acetone (150 mL) was added 1,2-dibromoethane (10 g, 52.9 mmol) at RT. The mixture was stirred at 60° C. for 4 days. The mixture was quenched with water (200 mL) and extracted with EA (200 mL×3). The combined organic phase was washed with brine, dried over Na₂SO₄ and concentrated under reduced pressure. The residue was purified by silica gel chromatography (petroleum ether/EA=3:1) to afford the title compound as yellow oil (4 g, yield: 41.4%). MS: M/e 385 (M+1)⁺.

### Step B: tert-butyl 4-(4-(2-(methylamino)ethoxy)phenyl)piperazine-1-carboxylate

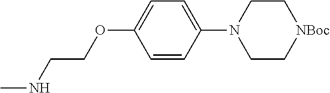 [SMILES: CNCCOc1ccc(N2CCN(C(=O)OC(C)(C)C)CC2)cc1]

To a stirred solution of the product of Step A (1 g, crude) in CH₃CN was added methylamine (2.5 mL, 40% aq) at RT. The mixture was heated to 95° C. in a sealed tube for 12 hours. The reaction was cooled to RT and concentrated under reduced pressure. The residue was dissolved into water (10 mL) and extracted with DCM (20 mL×2). The combined organic phase was washed with brine, dried over Na₂SO₄ and concentrated under reduced pressure. The residue (1 g, crude) as yellow oil was used into next step directly. MS: M/e 336 (M+1)⁺.

### Step C: N-methyl-2-(4-(piperazin-1-yl)phenoxy)ethan-1-amine Hydrochloride

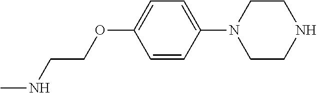 [SMILES: CNCCOc1ccc(N2CCNCC2)cc1]

The mixture of the product of Step B (1.2 g, crude) in HCl/1,4-dioxane (4M, 20 mL) was stirred at RT for 4 hours. The solid was precipitated from the system. The mixture was filtered and the solid was washed with EA (20 mL). The solid (750 mg, yield for two steps: 87.2%) was dried in air and used into next step directly. MS: M/e 236 (M+1)⁺.

### Step D: 2-(5-amino-2-(furan-2-yl)-7H-pyrazolo[4,3-e][1,2,4]triazolo[1,5-c]pyrimidin-7-yl)-1-(4-(4-(2-(methylamino)ethoxy)phenyl)piperazin-1-yl)propan-1-one

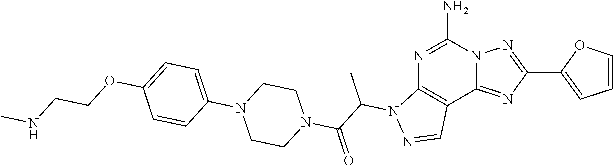 [SMILES: CNCCOc1ccc(N2CCN(C(=O)C(C)n3ncc4c3nc(N)n3nc(-c5ccco5)nc43)CC2)cc1]

A mixture of the product of Step C (294 mg, 0.96 mmol), 2-(5-amino-2-(furan-2-yl)-7H-pyrazolo[4,3-e][1,2,4]triazolo[1,5-c]pyrimidin-7-yl)propanoic acid (200 mg, 0.64 mmol), HATU (364 mg, 0.96 mmol) and DIEA (1 mL, excess) in DMF (20 mL) was stirred at RT overnight. The reaction mixture was poured into water (20 mL) and the solid was precipitated from the system. The solid was filtered and purified by prep-HPLC to afford the title compound (25 mg, yield: 7.4%). ¹H NMR (400 MHz, DMSO-d6) δ 9.22 (br.s, 2H), 8.81 (s, 1H), 7.96 (s, 1H), 7.80 (br.s, 2H), 7.64-7.49 (m, 2H), 7.24-7.15 (m, 1H), 7.13-6.99 (m, 2H), 6.74 (s, 1H), 6.07-5.82 (m, 1H), 4.37-4.20 (m, 2H), 4.17-3.75 (m, 4H), 3.56-3.23 (m, 6H), 2.60 (t, J=4 Hz, 3H), 1.73 (d, J=8 Hz, 3H) ppm. MS: M/e 531 (M+1)⁺.

### Compound A31: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(2-methoxyphenyl)ethan-1-one

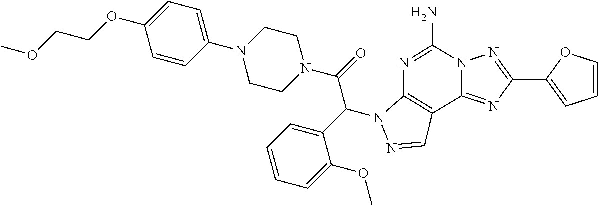 [SMILES: COCCOc1ccc(N2CCN(C(=O)C(c3ccccc3OC)n3ncc4c3nc(N)n3nc(-c5ccco5)nc43)CC2)cc1]

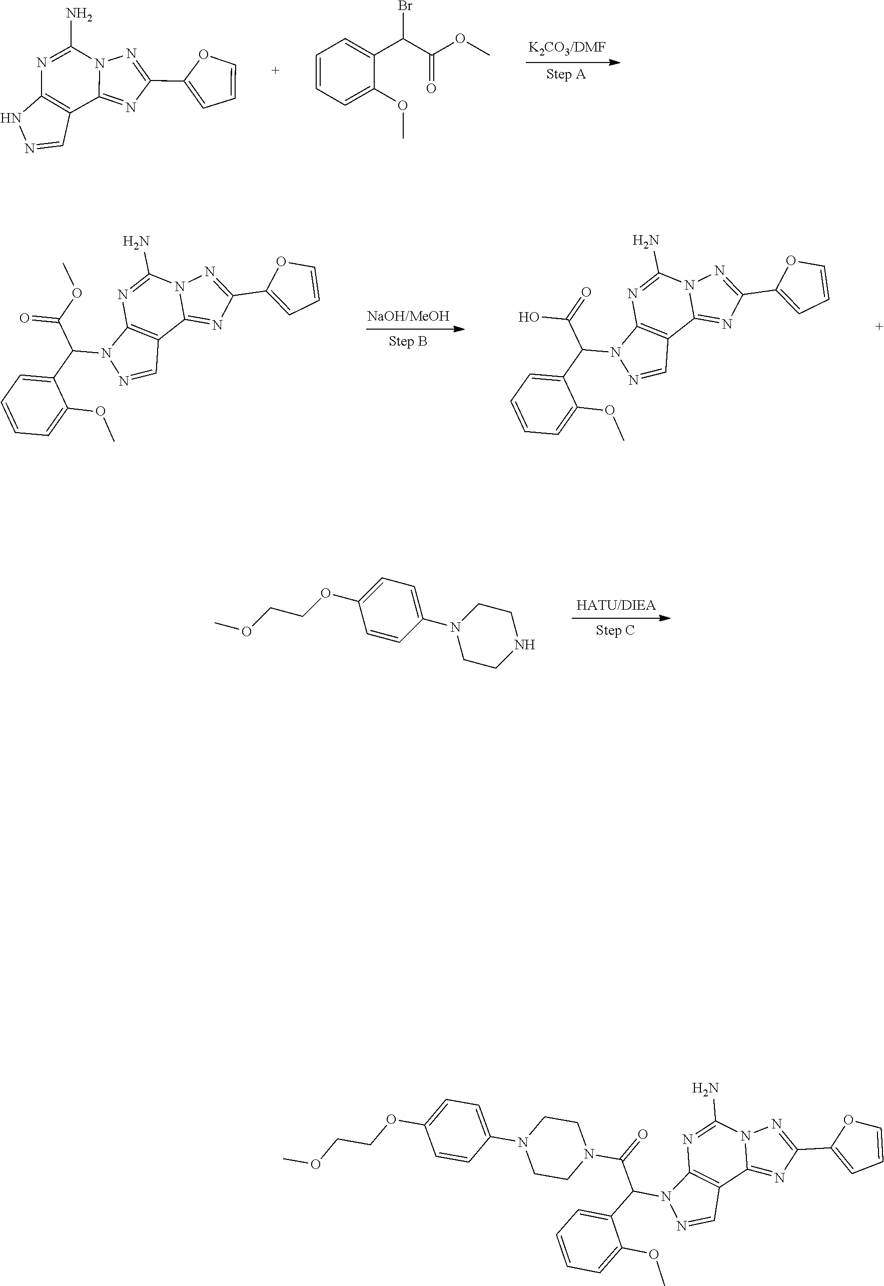 [SMILES: Nc1nc2[nH]ncc2c2nc(-c3ccco3)nn12 ; COC(=O)C(Br)c1ccccc1OC ; COC(=O)C(c1ccccc1OC)n1ncc2c1nc(N)n1nc(-c3ccco3)nc21 ; COc1ccccc1C(C(=O)O)n1ncc2c1nc(N)n1nc(-c3ccco3)nc21 ; COCCOc1ccc(N2CCNCC2)cc1 ; COCCOc1ccc(N2CCN(C(=O)C(c3ccccc3OC)n3ncc4c3nc(N)n3nc(-c5ccco5)nc43)CC2)cc1]

### Step A: methyl 2-(5-amino-2-(furan-2-yl)-7H-pyrazolo[4,3-e][1,2,4]triazolo[1,5-c]pyrimidin-7-yl)-2-(2-methoxyphenyl)acetate

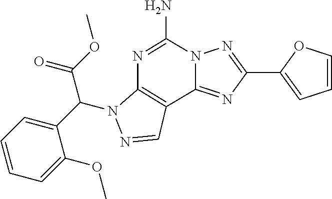 [SMILES: COC(=O)C(c1ccccc1OC)n1ncc2c1nc(N)n1nc(-c3ccco3)nc21]

A mixture of 2-(furan-2-yl)-7H-pyrazolo[4,3-e][1,2,4]triazolo[1,5-c]pyrimidin-5-amine (8 g, 33.1 mmol), methyl 2-bromo-2-(2-methoxyphenyl)acetate (9.4 g, 36.4 mmol) and K₂CO₃ (9.1 g, 66.2 mmol) in DMF (100 mL) was stirred at rt overnight. The solution was added with water (60 mL), extracted with ethyl aceate (60 mL) and washed with brine (60 mL). The organic layer was dried over Na₂SO₄, concentrated and purified by column chromatography (PE:EA=3:1 to 1:1) to get methyl 2-(5-amino-2-(furan-2-yl)-7H-pyrazolo[4,3-e][1,2,4]triazolo[1,5-c]pyrimidin-7-yl)-2-(2-methoxyphenyl)acetate (1.4 g, 10%). MS: M/e 420 (M+1)⁺

### Step B: 2-(5-amino-2-(furan-2-yl)-7H-pyrazolo[4,3-e][1,2,4]triazolo[1,5-c]pyrimidin-7-yl)-2-(2-methoxyphenyl)acetic Acid

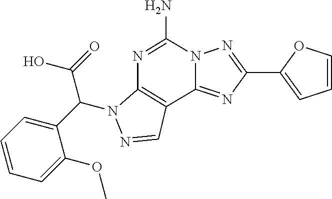 [SMILES: COc1ccccc1C(C(=O)O)n1ncc2c1nc(N)n1nc(-c3ccco3)nc21]

NaOH solution (240 mg, in 5 mL of water) was added to a solution of methyl 2-(5-amino-2-(furan-2-yl)-7H-pyrazolo[4,3-e][1,2,4]triazolo[1,5-c]pyrimidin-7-yl)-2-(2-methoxyphenyl)acetate (650 mg, 1.5 mmol) in methanol (30 mL). The reaction mixture was stirred at rt overnight. The solution was concentrated, added with water (20 mL) and acidified with 1N HCl solution to pH=5. The precipitated solid was filtered and dried to get the desired product as a white solid (450 mg, 72%). ¹H NMR (400 MHz, DMSO-d6) δ 13.22 (br.s, 1H), 8.25-8.15 (m, 3H), 7.95 (s, 1H), 7.36 (t, J=8.0 Hz, 1H), 7.25-7.22 (m, 2H), 7.07 (d, J=8.0 Hz, 1H), 6.96 (t, J=8.0 Hz, 1H), 6.76 (s, 1H), 6.75-6.73 (m, 1H), 3.80 (s, 3H) ppm. MS: M/e 406 (M+1)⁺

### Step C: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(2-methoxyphenyl)ethan-1-one

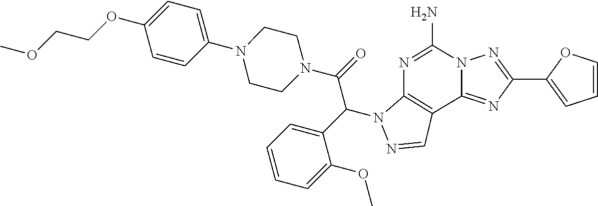 [SMILES: COCCOc1ccc(N2CCN(C(=O)C(c3ccccc3OC)n3ncc4c3nc(N)n3nc(-c5ccco5)nc43)CC2)cc1]

A mixture of 2-(5-amino-2-(furan-2-yl)-7H-pyrazolo[4,3-e][1,2,4]triazolo[1,5-c]pyrimidin-7-yl)-2-(2-methoxyphenyl)acetic acid (450 mg, 1.1 mmol), 1-(4-(2-methoxyethoxy)phenyl)piperazine (314 mg, 1.3 mmol), HATU (494 mg, 1.30 mmol) and DIEA (284 mg, 2.2 mmol) in DMF (50 mL) was stirred at rt for 2 hrs. The solution was added with water (30 mL), extracted with ethyl acetate (30 mL) and washed with brine (30 mL). The organic layer was dried, concentrated and purified by column chromatography (PE:EA=1:1 to EA) to get the desired product (480 mg, 70%). ¹H NMR (400 MHz, DMSO-d6) δ 8.20 (br.s, 3H), 7.95 (s, 1H), 7.38-7.34 (m, 1H), 7.25 (d, J=4.0 Hz, 1H), 7.09-7.06 (m, 2H), 7.00 (s, 1H), 6.95 (t, J=8.0 Hz, 1H), 6.79 (s, 4H), 6.75-6.73 (m, 1H), 3.98 (t, J=4.0 Hz, 2H), 3.81 (s, 3H), 3.80-3.74 (m, 1H), 3.61-3.53 (m, 3H), 3.37-3.35 (m, 1H), 3.30-3.27 (m, 4H), 3.05-2.95 (m, 1H), 2.90-2.80 (m, 1H), 2.73-2.65 (m, 1H), 2.47-2.50 (m, 1H) ppm. MS: M/e 624 (M+1)⁺

Compound A31 was separated into two enantiomeric stereoisomers (Compound A31a, earlier peak, and Compound A31b, later peak) by chiral prep-HPLC. The chiral separation conditions are shown below.

| Column | CHIRALPAK IG |
|---|---|
| Column size | 2 cm × 25 cm, 5 um |
| Injection | 0.5 mL |
| Mobile phase | MeOH:DCM = 80:20 |
| Flow rate | 20 ml/min |
| Wavelength | UV 220 nm |
| Temperature | 25° C. |
| Sample solution | 30.4 mg/ml in MeOH:DCM = 3:1 |
| Prep-HPLC equipment | Prep-Gilson-HPLC |

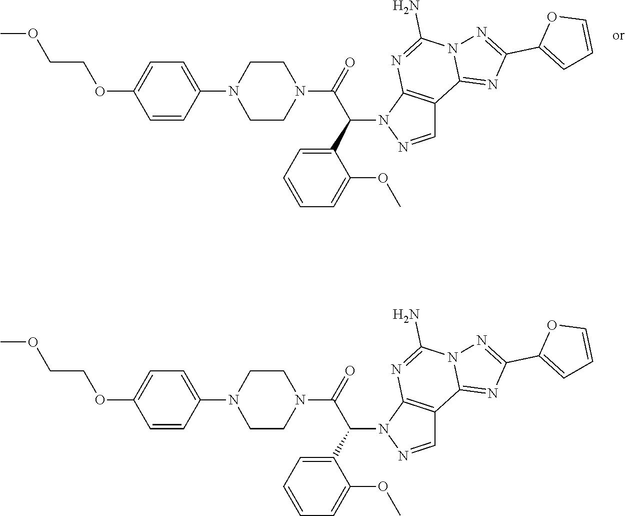 [SMILES: COCCOc1ccc(N2CCN(C(=O)[C@H](c3ccccc3OC)n3ncc4c3nc(N)n3nc(-c5ccco5)nc43)CC2)cc1 ; COCCOc1ccc(N2CCN(C(=O)[C@@H](c3ccccc3OC)n3ncc4c3nc(N)n3nc(-c5ccco5)nc43)CC2)cc1]

### Compound A32: 2-(5-amino-2-(furan-2-yl)-7H-pyrazolo[4,3-e][1,2,4]triazolo[1,5-c]pyrimidin-7-yl)-1-(4-(4-(2-(dimethylamino)ethoxy)phenyl)piperazin-1-yl)hexan-1-one

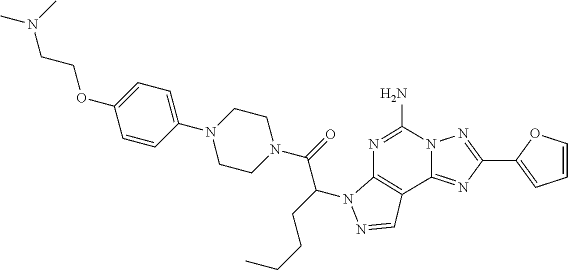 [SMILES: CCCCC(C(=O)N1CCN(c2ccc(OCCN(C)C)cc2)CC1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)hexanoic acid (50 mg, 0.14 mmol), N,N-dimethyl-2-(4-(piperazin-1-yl)phenoxy)ethan-1-amine hydrochloride (40 mg, 0.14 mmol), HATU (64 mg, 0.17 mmol) and DIEA (36 mg, 0.28 mmol) in DMF (5 mL) was stirred at rt for 2 hrs. The solution was added with water (5 mL), extracted with ethyl acetate (10 mL) and washed with brine (10 mL). The organic layer was dried, concentrated and purified by preparative HPLC to get the desired product as a TFA salt (15 mg, 18%). ¹H NMR (400 MHz, DMSO-d6) δ 9.65 (br.s, 1H), 8.20 (s, 1H), 8.18 (br.s, 2H), 7.95 (s, 1H), 7.23 (d, J=4.0 Hz, 1H), 6.85-6.74 (m, 5H), 5.56 (dd, J=8.0 Hz, 4.0 Hz, 1H), 4.20 (t, J=4.0 Hz, 2H), 3.74 (br.s, 1H), 3.53-3.46 (m, 5H), 3.05-2.96 (m, 2H), 2.78-2.65 (m, 7H), 2.33 (br.s, 1H), 2.16-2.07 (m, 2H), 1.28-1.23 (m, 3H), 1.03-1.01 (m, 1H), 0.81 (t, J=8.0 Hz, 3H) ppm. MS: M/e 587 (M+1)⁺

### Compound A33: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(naphthalen-2-yl)ethan-1-one

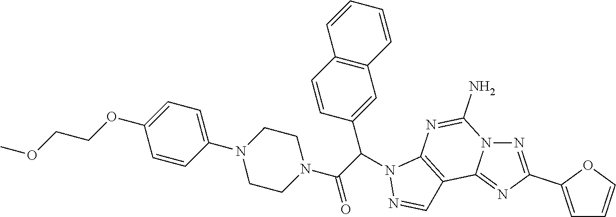 [SMILES: COCCOc1ccc(N2CCN(C(=O)C(c3ccc4ccccc4c3)n3ncc4c3nc(N)n3nc(-c5ccco5)nc43)CC2)cc1]

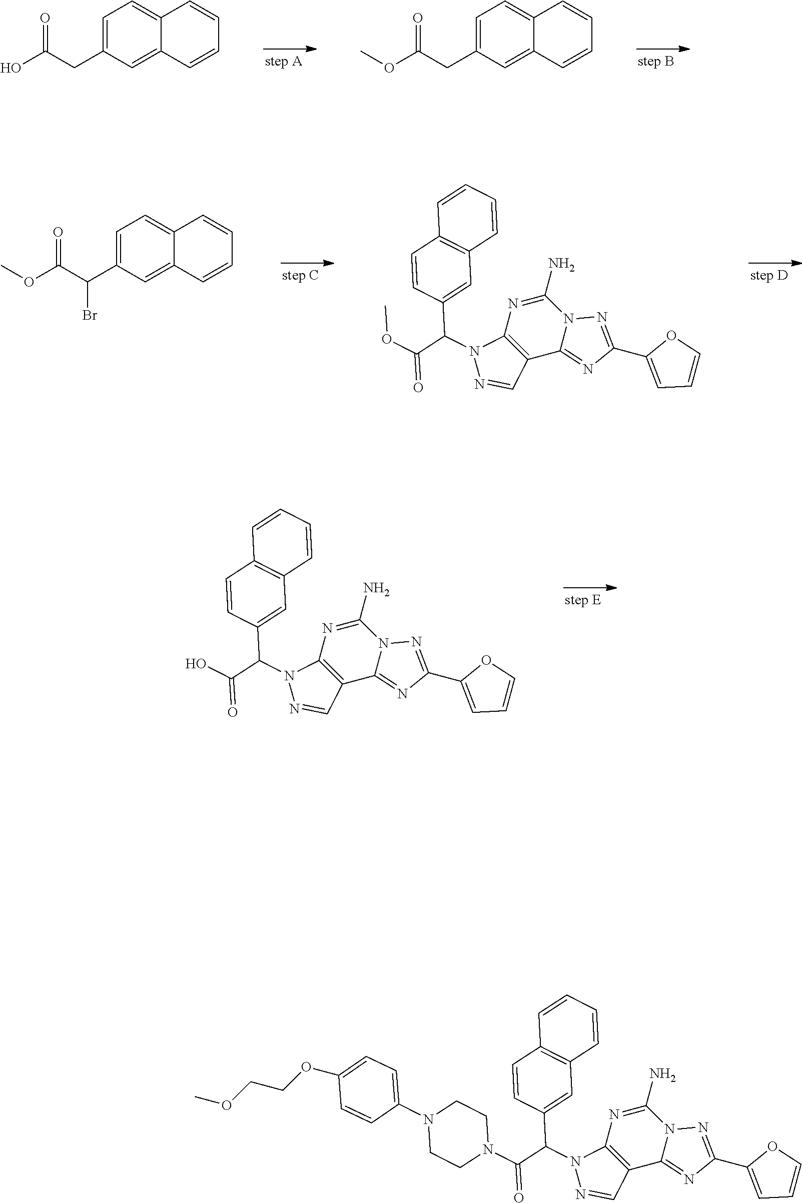 [SMILES: O=C(O)Cc1ccc2ccccc2c1 ; COC(=O)Cc1ccc2ccccc2c1 ; COC(=O)C(Br)c1ccc2ccccc2c1 ; COC(=O)C(c1ccc2ccccc2c1)n1ncc2c1nc(N)n1nc(-c3ccco3)nc21 ; Nc1nc2c(cnn2C(C(=O)O)c2ccc3ccccc3c2)c2nc(-c3ccco3)nn12 ; COCCOc1ccc(N2CCN(C(=O)C(c3ccc4ccccc4c3)n3ncc4c3nc(N)n3nc(-c5ccco5)nc43)CC2)cc1]

### Step A: methyl 2-(naphthalen-2-yl)acetate

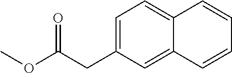

To a stirred solution of 2-(naphthalen-2-yl)acetic acid (5 g, 26.7 mmol) in MeOH (100 mL) was added dropwise SOCl₂ (4 g, 29.6 mmol) at 0° C. After the addition, the reaction mixture was stirred overnight. Most of the MeOH was removed to give the residue, then treated with EtOAc (50 mL) and washed with aq.K₂CO₃, brine, dried over Na₂SO₄, concentrated to give the target compound (4.5 g, 84.3%) as yellow oil.

### Step B: methyl 2-bromo-2-(naphthalen-2-yl)acetate

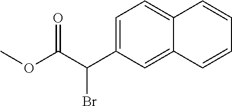 [SMILES: COC(=O)C(Br)c1ccc2ccccc2c1]

To a stirred solution of the product of step A (1 g, 5 mmol) in CCl₄ (10 mL) was added NBS (0.95 g, 5.5 mmol) and added aq.HBr (48%, 2 drops). Then the mixture was refluxed for 2 hours. The reaction mixture was allowed to cool to room temperature and filtered. The filtrate was concentrated, purified by column chromatography (petroleum ether/EtOAc=20:1) to give the target compound (0.72 g, 51.6%) as colorless oil.

### Step C: methyl 2-(5-amino-2-(furan-2-yl)-7H-pyrazolo[4,3-e][1,2,4]triazolo[1,5-c]pyrimidin-7-yl)-2-(naphthalen-2-yl)acetate

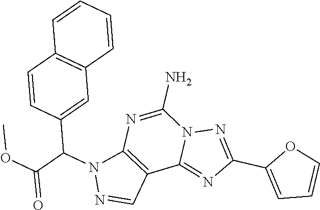 [SMILES: COC(=O)C(c1ccc2ccccc2c1)n1ncc2c1nc(N)n1nc(-c3ccco3)nc21]

To a stirred solution of 2-(furan-2-yl)-7H-pyrazolo[4,3-e][1,2,4]triazolo[1,5-c]pyrimidin-5-amine (0.62 g, 2.58 mmol) in DMF (5 mL) was added K₂CO₃ (0.71 g, 5.16 mmol), then the product of step B (0.72 g, 2.58 mmol) was added. After the addition, the reaction mixture was stirred for a weekend. The reaction mixture was poured into H₂O (25 mL) and extracted with EtOAc (15 mL×4). The combined organic layers were washed with brine, dried over Na₂SO₄, concentrated and purified by column chromatography (petroleum ether/EtOAc=1:1˜100% EtOAc) to give the target compound (80 mg, 7%) as a white solid. MS: M/e 440 (M+1)⁺.

### Step D: 2-(5-amino-2-(furan-2-yl)-7H-pyrazolo[4,3-e][1,2,4]triazolo[1,5-c]pyrimidin-7-yl)-2-(naphthalen-2-yl)acetic Acid

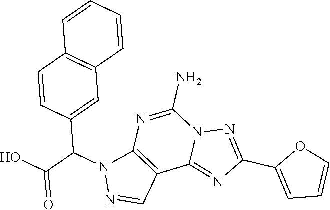 [SMILES: Nc1nc2c(cnn2C(C(=O)O)c2ccc3ccccc3c2)c2nc(-c3ccco3)nn12]

To a stirred solution of the product of step C (80 mg, 0.18 mmol) in MeOH (15 mL) was added aq.NaOH (2.0 M, 3 mL). After the addition, the reaction mixture was stirred overnight. Most of MeOH was removed to give aqueous layer, then acidified to pH=3˜4 and filtered. The cake was collected, dried to give the target compound (70 mg, 91.5%) as a white solid. MS: M/e 426 (M+1)⁺.

### Step E: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(naphthalen-2-yl)ethan-1-one

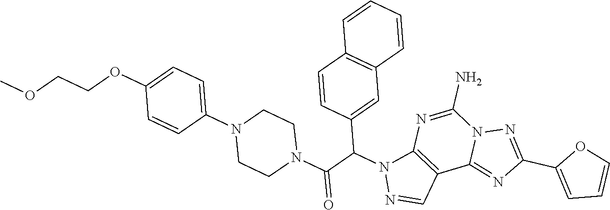 [SMILES: COCCOc1ccc(N2CCN(C(=O)C(c3ccc4ccccc4c3)n3ncc4c3nc(N)n3nc(-c5ccco5)nc43)CC2)cc1]

A mixture of the product of step D (70 mg, 0.164 mmol), 1-(4-(2-methoxyethoxy)phenyl)piperazine (38.8 mg, 0.164 mmol), HATU (75 mg, 0.196 mmol) and DIPEA (42.3 mg, 0.328 mmol) in DMF (4 mL) was stirred overnight. The reaction mixture was poured into H₂O (20 mL), and extracted with EtOAc (20 mL×3). The combined organic layers were washed with brine, dried over Na₂SO₄, concentrated and purified by column chromatography (petroleum ether/EtOAc=2:1˜1:2) to give the target compound (60 mg, 56.8%). ¹H NMR (400 MHz, DMSO-d6) δ 8.22 (s, 1H), 8.17 (s, 2H), 7.92-7.87 (m, 5H), 7.61-7.49 (m, 3H), 7.24 (d, J=3.2 Hz, 1H), 7.04 (s, 1H), 6.78 (s, 4H), 6.74 (s, 1H), 3.99-3.93 (m, 2H), 3.71 (m, 2H), 3.60 (m, 2H), 3.40 (m, 2H), 3.28 (s, 3H), 2.99 (m, 2H), 2.64 (m, 2H) ppm. MS: M/e 644 (M+1)⁺.

### Compound A34: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(4-(trifluoromethyl)phenyl)ethan-1-one

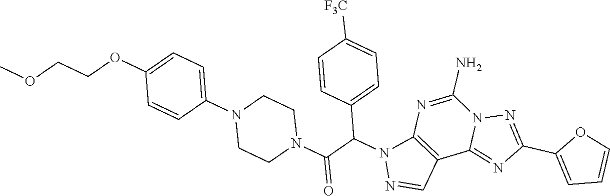 [SMILES: COCCOc1ccc(N2CCN(C(=O)C(c3ccc(C(F)(F)F)cc3)n3ncc4c3nc(N)n3nc(-c5ccco5)nc43)CC2)cc1]

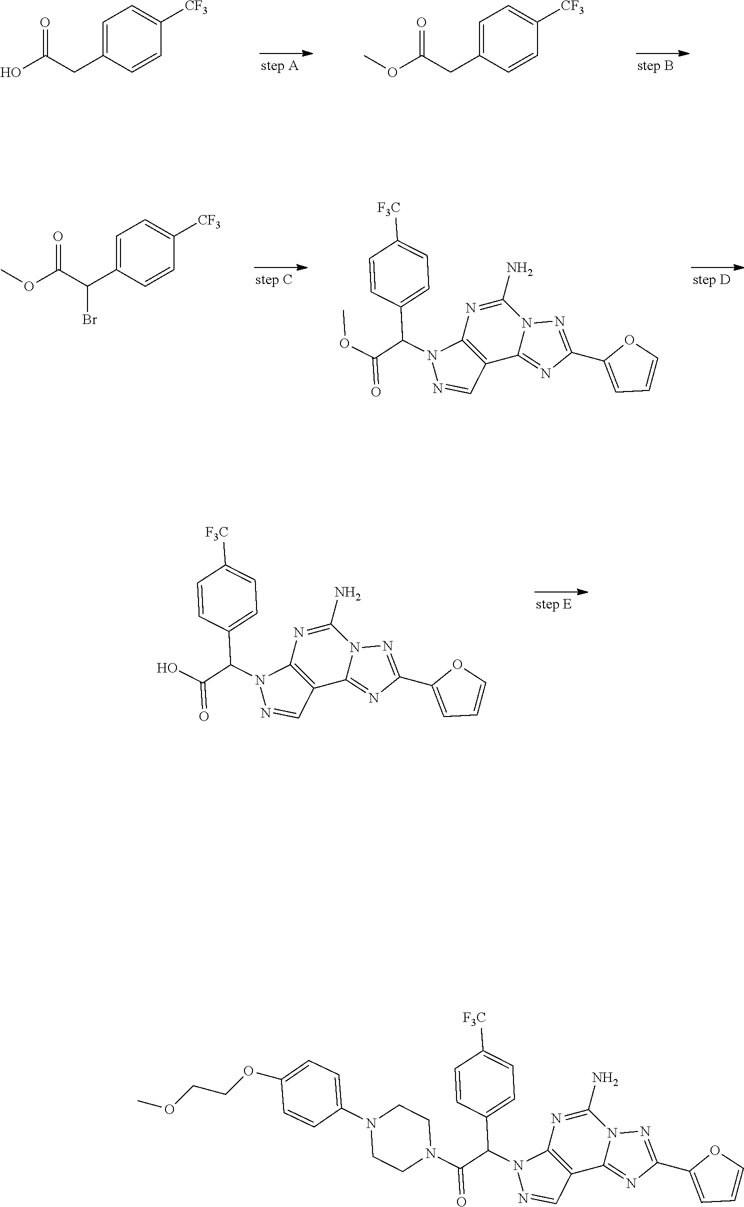 [SMILES: O=C(O)Cc1ccc(C(F)(F)F)cc1 ; COC(=O)Cc1ccc(C(F)(F)F)cc1 ; COC(=O)C(Br)c1ccc(C(F)(F)F)cc1 ; COC(=O)C(c1ccc(C(F)(F)F)cc1)n1ncc2c1nc(N)n1nc(-c3ccco3)nc21 ; Nc1nc2c(cnn2C(C(=O)O)c2ccc(C(F)(F)F)cc2)c2nc(-c3ccco3)nn12 ; COCCOc1ccc(N2CCN(C(=O)C(c3ccc(C(F)(F)F)cc3)n3ncc4c3nc(N)n3nc(-c5ccco5)nc43)CC2)cc1]

### Step A: methyl 2-(4-(trifluoromethyl)phenyl)acetate

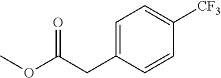

To a stirred solution of 2-(4-(trifluoromethyl)phenyl)acetic acid (5 g, 24.5 mmol) in MeOH (50 mL) was added dropwise SOCl₂ (3.45 g, 29.45 mmol) at 0° C. After the addition, the reaction mixture was stirred overnight. Most of the MeOH was removed to give the residue, then treated with EtOAc (20 mL×2) and washed with aq.K₂CO₃, brine, dried over Na₂SO₄, concentrated to give the target compound (5.3 g, 99%) as colorless oil.

### Step B: methyl 2-bromo-2-(4-(trifluoromethyl)phenyl)acetate

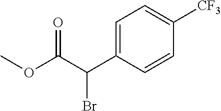 [SMILES: COC(=O)C(Br)c1ccc(C(F)(F)F)cc1]

To a stirred solution of the product of step A (1 g, 4.58 mmol) in CCl₄ (15 mL) was added NBS (0.9 g, 5.04 mmol) and added aq.HBr (48%, 2 drops). Then the mixture was refluxed for 3 hours. The reaction mixture was allowed to cool to room temperature and filtered. The filtrate was concentrated to give the target compound (1.3 g, 95.6%) as colorless oil.

### Step C: methyl 2-(5-amino-2-(furan-2-yl)-7H-pyrazolo[4,3-e][1,2,4]triazolo[1,5-c]pyrimidin-7-yl)-2-(4-(trifluoromethyl)phenyl)acetate

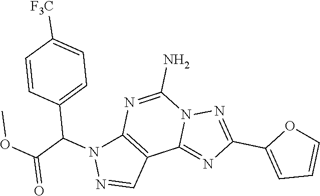 [SMILES: COC(=O)C(c1ccc(C(F)(F)F)cc1)n1ncc2c1nc(N)n1nc(-c3ccco3)nc21]

To a stirred solution of 2-(furan-2-yl)-7H-pyrazolo[4,3-e][1,2,4]triazolo[1,5-c]pyrimidin-5-amine (0.482 g, 2. mmol) in DMF (5 mL) was added K₂CO₃ (0.552 g, 4 mmol), then the product of step B (0.594 g, 2 mmol) was added. After the addition, the reaction mixture was stirred overnight. The reaction mixture was poured into H₂O (20 mL) and extracted with EtOAc (20 mL×3). The combined organic layers were washed with brine, dried over Na₂SO₄, concentrated and purified by column chromatography (petroleum ether/EtOAc=2:1˜1:2) to give the target compound (140 mg, 15.3%) as a white solid. MS: M/e 458 (M+1)⁺.

### Step D: 2-(5-amino-2-(furan-2-yl)-7H-pyrazolo[4,3-e][1,2,4]triazolo[1,5-c]pyrimidin-7-yl)-2-(4-(trifluoromethyl)phenyl)acetic Acid

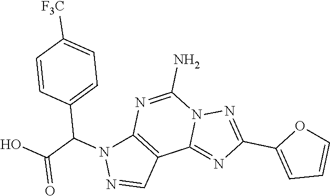 [SMILES: Nc1nc2c(cnn2C(C(=O)O)c2ccc(C(F)(F)F)cc2)c2nc(-c3ccco3)nn12]

To a stirred solution of the product of step C (140 mg, 0.3 mmol) in MeOH (15 mL) was added aq.NaOH (2.0 M, 4 mL). After the addition, the reaction mixture was stirred overnight. Most of MeOH was removed to give an aqueous layer, then acidified to pH=3˜4 and filtered. The cake was collected, dried to give the target compound (109 mg, 80%) as a white solid. MS: M/e 444 (M+1)⁺.

### Step E: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(4-(trifluoromethyl)phenyl)ethan-1-one

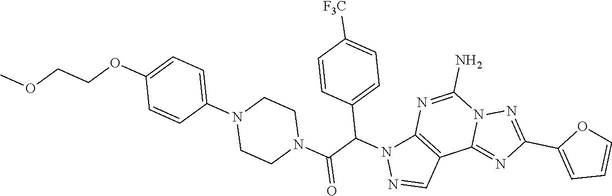 [SMILES: COCCOc1ccc(N2CCN(C(=O)C(c3ccc(C(F)(F)F)cc3)n3ncc4c3nc(N)n3nc(-c5ccco5)nc43)CC2)cc1]

A mixture of the product of step D (44 mg, 0.1 mmol), 1-(4-(2-methoxyethoxy)phenyl)piperazine (23.6 mg, 0.1 mmol), HATU (45.8 mg, 0.12 mmol) and DIPEA (25.8 mg, 0.2 mmol) in DMF (3 mL) was stirred overnight. The reaction mixture was poured into H₂O (20 mL), and extracted with EtOAc (20 mL×3). The combined organic layers were washed with brine, dried over Na₂SO₄, concentrated and purified by column chromatography (petroleum ether/EtOAc=2:1˜1:1) to give the target compound (48 mg, 72.6%). ¹H NMR (400 MHz, DMSO-d6) δ 8.28 (s, 1H), 8.19 (s, 2H), 7.95 (s, 1H), 7.74 (d, J=8.0 Hz, 2H), 7.56 (d, J=8.0 Hz, 2H), 7.24 (d, J=3.2 Hz, 1H), 7.01 (s, 1H), 6.82-6.74-6.83 (m, 3H), 6.74 (s, 1H), 4.00-3.95 (m, 2H), 3.75-3.58 (m, 4H), 3.40 (m, 1H), 3.28 (s, 3H), 3.08-2.90 (m, 2H), 2.76 (m, 1H), 2.56 (m, 1H) ppm. M/e 662 (M+1)⁺.

### Compound A35: 2-(5-amino-2-(furan-2-yl)-7H-pyrazolo[4,3-e][1,2,4]triazolo[1,5-c]pyrimidin-7-yl)-1-(4-(4-(2-(methylamino)ethoxy)phenyl)piperazin-1-yl)hexan-1-one

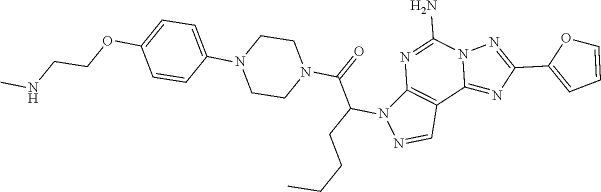 [SMILES: CCCCC(C(=O)N1CCN(c2ccc(OCCNC)cc2)CC1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)hexanoic acid (50 mg, 0.14 mmol), N-methyl-2-(4-(piperazin-1-yl)phenoxy)ethan-1-amine hydrochloride (38 mg, 0.14 mmol), HATU (64 mg, 0.17 mmol) and DIEA (36 mg, 0.28 mmol) in DMF (5 mL) was stirred at rt for 4 hrs. The solution was concentrated and purified by preparative HPLC to get the product as a TFA salt (5 mg, 6%). ¹H NMR (400 MHz, DMSO-d6) δ 8.54 (br.s, 2H), 8.20 (s, 1H), 8.18 (br.s, 2H), 7.95 (s, 1H), 7.23 (d, J=4.0 Hz, 1H), 6.85-6.74 (m, 5H), 5.56 (dd, J=8.0 Hz, 4.0 Hz, 1H), 4.10 (t, J=4.0 Hz, 2H), 3.74-3.53 (m, 4H), 3.28-2.79 (m, 5H), 2.62 (t, J=4.0 Hz, 3H), 2.33-2.07 (m, 3H), 1.28-1.23 (m, 3H), 1.02 (br.s, 1H), 0.81 (t, J=8.0 Hz, 3H) ppm. MS: M/e 573 (M+1)⁺

### Compound A36: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(3-(trifluoromethyl)phenyl)ethan-1-one

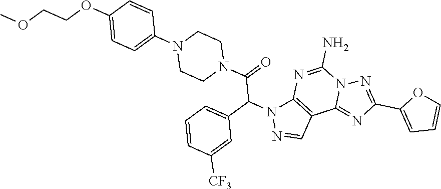 [SMILES: COCCOc1ccc(N2CCN(C(=O)C(c3cccc(C(F)(F)F)c3)n3ncc4c3nc(N)n3nc(-c5ccco5)nc43)CC2)cc1]

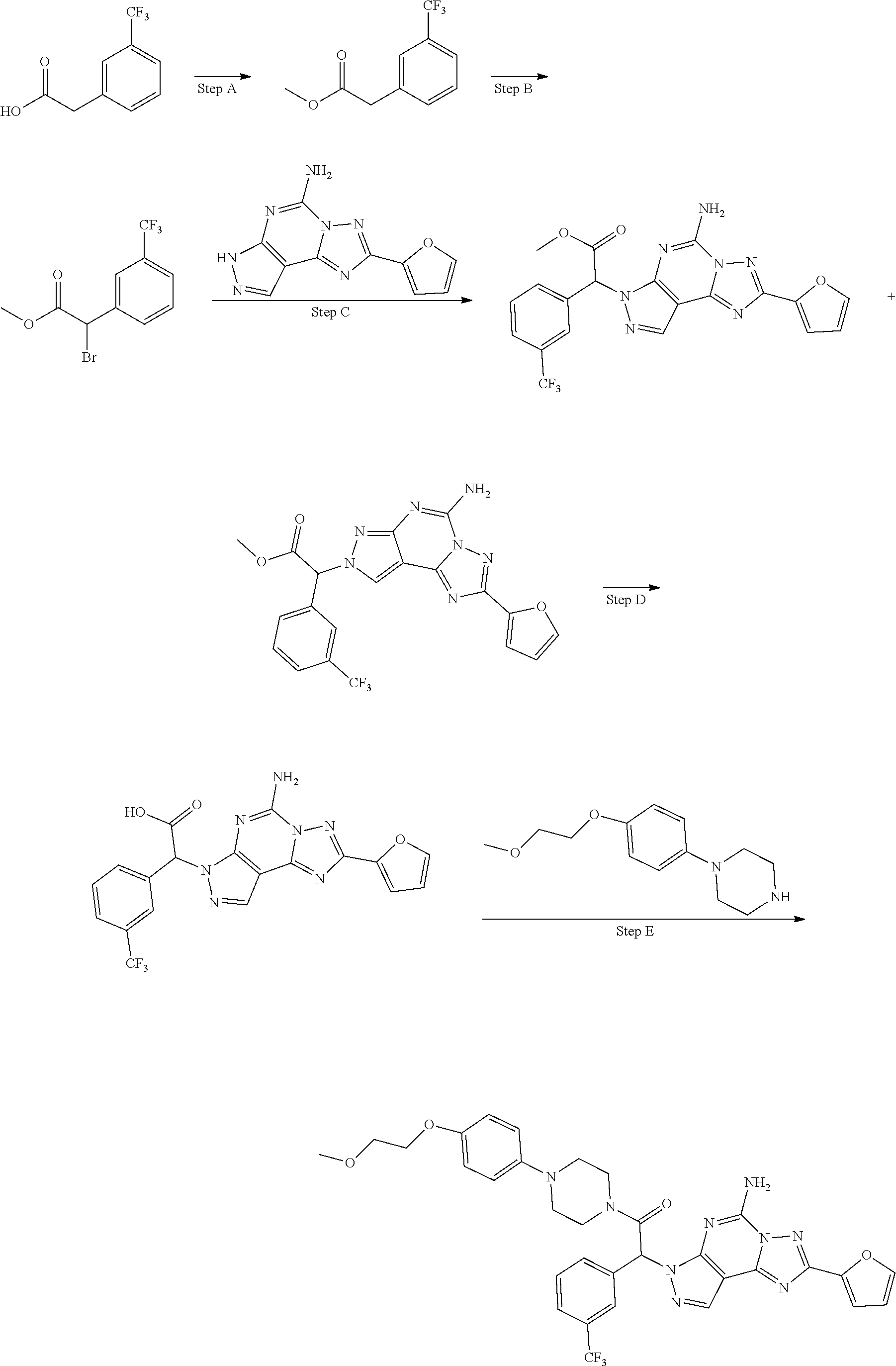

### Step A: methyl 2-(3-(trifluoromethyl)phenyl)acetate

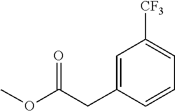

To a stirred solution of 2-(3-(trifluoromethyl)phenyl)acetic acid (5.0 g, 24.5 mmol) in MeOH (50 mL) was added SOCl₂ (2 mL) in drops at rt and the resulting mixture was stirred for 3 hrs. The mixture was concentrated, diluted with EA (50 mL), washed with aqueous solution of NaHCO₃ (50 mL×2), brine (50 mL×2), dried over Na₂SO₄ and concentrated to give the title product (5.05 g, 95%) as a light yellow oil. MS: M/e 219 (M+1)⁺.

### Step B: methyl 2-bromo-2-(3-(trifluoromethyl)phenyl)acetate

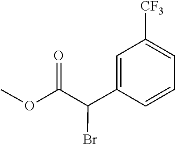 [SMILES: COC(=O)C(Br)c1cccc(C(F)(F)F)c1]

To a stirred solution of methyl 2-(3-(trifluoromethyl)phenyl)acetate (2.0 g, 9.2 mmol) in CCl₄ (20 mL) was added NBS (1.9 g, 10.7 mmol) and followed by HBr/AcOH (3 drops). The resulting mixture was refluxed for 4 hrs. The mixture was filtered and the filtrate was concentrated, diluted with EA (30 mL), washed with brine (20 mL×2), dried over Na₂SO₄ and concentrated to give the title product (2.36 g, crude) as a light yellow oil. MS: M/e 297, 299 (M+1)⁺.

### Step C: methyl 2-(5-amino-2-(furan-2-yl)-7H-pyrazolo[4,3-e][1,2,4]triazolo[1,5-c]pyrimidin-7-yl)-2-(3-(trifluoromethyl)phenyl)acetate and methyl 2-(5-amino-2-(furan-2-yl)-8H-pyrazolo[4,3-e][1,2,4]triazolo[1,5-c]pyrimidin-8-yl)-2-(3-(trifluoromethyl)phenyl)acetate

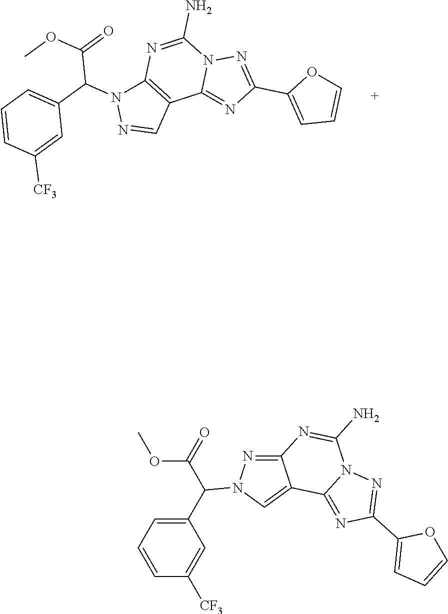 [SMILES: COC(=O)C(c1cccc(C(F)(F)F)c1)n1ncc2c1nc(N)n1nc(-c3ccco3)nc21 ; COC(=O)C(c1cccc(C(F)(F)F)c1)n1cc2c(nc(N)n3nc(-c4ccco4)nc23)n1]

To a stirred solution of 2-(furan-2-yl)-7H-pyrazolo[4,3-e][1,2,4]triazolo[1,5-c]pyrimidin-5-amine (250 mg, 1.0 mmol) in DMF (50 mL) was added K₂CO₃ (550 Mg, 3.6 mmol) and followed by methyl 2-bromo-2-(3-(trifluoromethyl)phenyl)acetate (900 mg, crude, about 1 mmol). The resulting mixture was stirred at 50° C. for 16 hrs. The mixture was diluted with CH₂Cl₂ (30 mL), washed with brine (15 mL×3), dried over Na₂SO₄ and concentrated. The resulting residue was purified by column chromatography to give methyl 2-(5-amino-2-(furan-2-yl)-7H-pyrazolo[4,3-e][1,2,4]triazolo[1,5-c]pyrimidin-7-yl)-2-(3-(trifluoromethyl)phenyl)acetate (120 mg, crude) as a light yellow oil, and methyl 2-(5-amino-2-(furan-2-yl)-8H-pyrazolo[4,3-e][1,2,4]triazolo[1,5-c]pyrimidin-8-yl)-2-(3-(trifluoromethyl)phenyl)acetate (60 mg, crude) as a light yellow solid. MS: M/e 458 (M+1)⁺.

### Step D: 2-(5-amino-2-(furan-2-yl)-7H-pyrazolo[4,3-e][1,2,4]triazolo[1,5-c]pyrimidin-7-yl)-2-(3-(trifluoromethyl)phenyl)acetic Acid

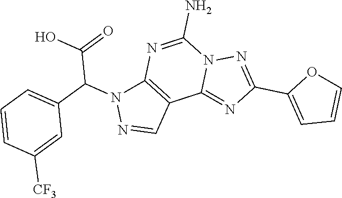 [SMILES: Nc1nc2c(cnn2C(C(=O)O)c2cccc(C(F)(F)F)c2)c2nc(-c3ccco3)nn12]

To a stirred solution of methyl 2-(5-amino-2-(furan-2-yl)-7H-pyrazolo[4,3-e][1,2,4]triazolo[1,5-c]pyrimidin-7-yl)-2-(3-(trifluoromethyl)phenyl)acetate (120 mg, crude) in MeOH (4 mL) was added aqueous solution of NaOH (2 M, 1 mL) at rt and the resulting mixture was stirred for 4 hrs. The mixture was neutralized by HCl (1 M) and concentrated to dryness. 5 mL of a mixed solvent (CH₂Cl₂/MeOH=3:1) was added and stirred for 10 min. The suspension was filtered and the filtrate was concentrated to give the title product (90 mg, crude) as a white solid. MS: M/e 444 (M+1)⁺.

### Step E: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(3-(trifluoromethyl)phenyl)ethan-1-one

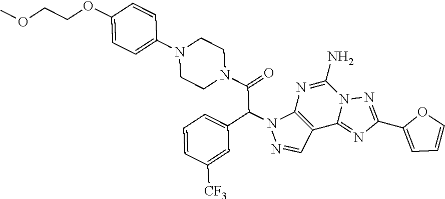 [SMILES: COCCOc1ccc(N2CCN(C(=O)C(c3cccc(C(F)(F)F)c3)n3ncc4c3nc(N)n3nc(-c5ccco5)nc43)CC2)cc1]

To a mixture of 2-(5-amino-2-(furan-2-yl)-7H-pyrazolo[4,3-e][1,2,4]triazolo[1,5-c]pyrimidin-7-yl)-2-(3-(trifluoromethyl)phenyl)acetic acid (90 mg, 0.2 mmol), 1-(4-(2-methoxyethoxy)phenyl)piperazine (58 mg, 0.24 mmol), DIEA (150 mg, 1.16 mmol) in DMF (2 mL) was added HATU (95 mg, 0.25 mmol) and the mixture was stirred at rt for 16 hrs. 20 mL of EA was added and the mixture was washed with brine (5 mL×3), dried over Na₂SO₄ and concentrated. The resulting residue was purified by prep-TLC to give the title product (18.0 mg, yield: 14%). ¹H NMR (400 MHz, DMSO-d6) δ 8.29 (s, 1H), 8.27-8.08 (m, 2H), 7.95 (s, 1H), 7.81 (s, 1H), 7.75-7.66 (m, 1H), 7.65-7.56 (m, 2H), 7.24 (d, J=3.2 Hz, 1H), 7.04 (s, 1H), 6.83-6.75 (m, 4H), 6.75-6.71 (m, 1H), 4.00-3.94 (m, 2H), 3.72-3.62 (m, 2H), 3.62-3.56 (m, 2H), 3.49-3.39 (m, 1H), 3.28 (s, 3H), 3.03-2.88 (m, 3H), 2.74-2.68 (m, 1H), 2.59-2.52 (m, 1H). MS: M/e 662 (M+1)⁺.

### Compound A37: 2-(5-amino-2-(furan-2-yl)-7H-pyrazolo[4,3-e][1,2,4]triazolo[1,5-c]pyrimidin-7-yl)-1-(4-(4-(2-(dimethylamino)ethoxy)phenyl)piperazin-1-yl)-2-phenylethan-1-one

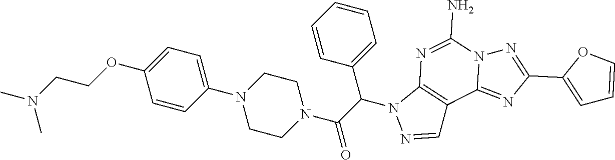 [SMILES: CN(C)CCOc1ccc(N2CCN(C(=O)C(c3ccccc3)n3ncc4c3nc(N)n3nc(-c5ccco5)nc43)CC2)cc1]

A mixture of N,N-dimethyl-2-(4-(piperazin-1-yl)phenoxy)ethan-1-amine hydrochloride (42 mg, 0.15 mmol), 2-(5-amino-2-(furan-2-yl)-7H-pyrazolo[4,3-e][1,2,4]triazolo[1,5-c]pyrimidin-7-yl)-2-phenylacetic acid (50 mg, 0.13 mmol), HATU (56 mg, 0.15 mmol) and DIEA (0.5 mL, excess) in DMF (5 mL) was stirred at RT overnight. The reaction mixture was poured into water (10 mL) and the solid was precipitated from the system. The solid was filtered and purified by prep-HPLC to afford the title compound (10 mg, yield: 12.4%). ¹H NMR (400 MHz, DMSO-d6) δ 8.23 (s, 1H), 8.16 (br.s, 2H), 7.95 (s, 1H), 7.44-7.31 (m, 5H), 7.24 (d, J=4 Hz, 1H), 7.16-7.03 (m, 2H), 7.01-6.92 (m, 2H), 6.88 (s, 1H), 6.78-6.71 (m, 1H), 4.30-4.21 (m, 2H), 3.88-3.64 (m, 2H), 3.60-3.42 (m, 4H), 3.22-3.09 (m, 2H), 2.88-2.80 (m, 6H), 2.80-2.73 (m, 1H) ppm. MS: M/e 607 (M+1)⁺.

### Compound A38: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(2-(trifluoromethyl)phenyl)ethan-1-one

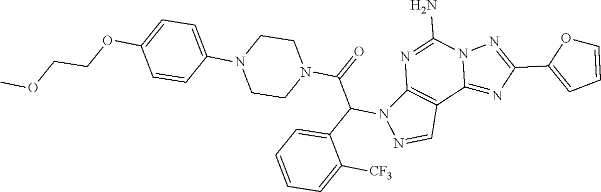 [SMILES: COCCOc1ccc(N2CCN(C(=O)C(c3ccccc3C(F)(F)F)n3ncc4c3nc(N)n3nc(-c5ccco5)nc43)CC2)cc1]

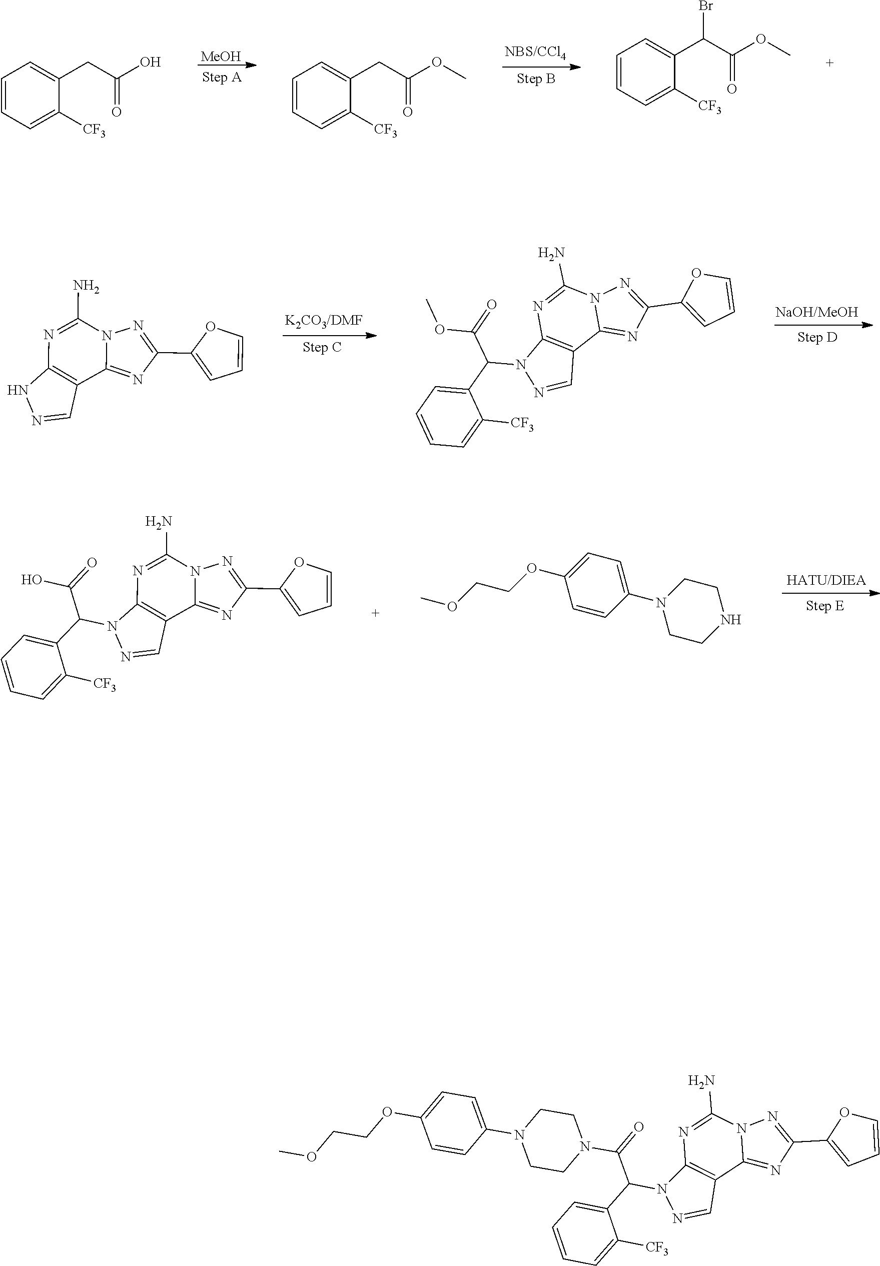

### Step A: methyl 2-(2-(trifluoromethyl)phenyl)acetate

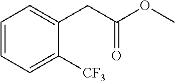

A solution of 2-(2-(trifluoromethyl)phenyl)acetic acid (5 g, 24.5 mmol) in methanol (30 mL) containing 5 drops of concentrated H₂SO₄ was heated at reflux for 2 hrs. The reaction mixture was evaporated, diluted with ethyl acetate (20 mL), washed with NaHCO₃ solution (20 mL) and brine (20 mL). The organic layer was dried and concentrated to give the product as an oil (4.7 g, 88%). ¹H NMR (400 MHz, DMSO-d6) δ 7.73-7.64 (m, 2H), 7.54-7.49 (m, 2H), 3.90 (s, 2H), 3.62 (s, 3H) ppm. MS: M/e 219 (M+1)⁺

### Step B: methyl 2-bromo-2-(2-(trifluoromethyl)phenyl)acetate

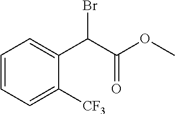 [SMILES: COC(=O)C(Br)c1ccccc1C(F)(F)F]

A mixture of methyl 2-(2-(trifluoromethyl)phenyl)acetate (2 g, 9 mmol), N-bromosuccinimide (1.7 g, 10 mmol) and AIBN (150 mg, 0.9 mmol) in CHCl₃ (15 mL) was heated at 80° C. overnight. The solution was concentrated and purified by column chromatography (PE:EA=10:1) to get the product (1.5 g, 56%). MS: M/e 297 (M+1)⁺

### Step C: methyl 2-(5-amino-2-(furan-2-yl)-7H-pyrazolo[4,3-e][1,2,4]triazolo[1,5-c]pyrimidin-7-yl)-2-(2-(trifluoromethyl)phenyl)acetate

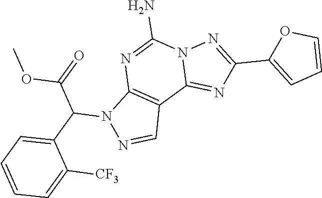 [SMILES: COC(=O)C(c1ccccc1C(F)(F)F)n1ncc2c1nc(N)n1nc(-c3ccco3)nc21]

A mixture of 2-(furan-2-yl)-7H-pyrazolo[4,3-e][1,2,4]triazolo[1,5-c]pyrimidin-5-amine (0.5 g, 2 mmol), methyl 2-bromo-2-(2-(trifluoromethyl)phenyl)acetate (733 mg, 2.5 mmol) and K₂CO₃ (552 mg, 4 mmol) in DMF (15 mL) was stirred at rt overnight. The solution was added with water (15 mL), extracted with ethyl aceate (15 mL) and washed with brine (20 mL). The organic layer was dried over Na₂SO₄, concentrated and purified by column chromatography (PE:EA=2:1) to get methyl 2-(5-amino-2-(furan-2-yl)-7H-pyrazolo[4,3-e][1,2,4]triazolo[1,5-c]pyrimidin-7-yl)-2-(2-(trifluoromethyl)phenyl)acetate (120 mg, 13%). MS: M/e 458 (M+1)⁺

### Step D: 2-(5-amino-2-(furan-2-yl)-7H-pyrazolo[4,3-e][1,2,4]triazolo[1,5-c]pyrimidin-7-yl)-2-(2-(trifluoromethyl)phenyl)acetic Acid

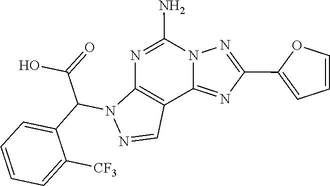 [SMILES: Nc1nc2c(cnn2C(C(=O)O)c2ccccc2C(F)(F)F)c2nc(-c3ccco3)nn12]

NaOH solution (52 mg, in 1 mL of water) was added to a solution of methyl 2-(5-amino-2-(furan-2-yl)-7H-pyrazolo[4,3-e][1,2,4]triazolo[1,5-c]pyrimidin-7-yl)-2-(2-(trifluoromethyl)phenyl)acetate (120 mg, 0.26 mmol) in methanol (5 mL). The reaction mixture was stirred at rt overnight. The solution was concentrated, added with water (5 mL) and acidified with 1N HCl solution to pH=5. The precipitated solid was filtered and dried to get the desired product as a white solid (100 mg, 86%). MS: M/e 444 (M+1)⁺

### Step E: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(2-(trifluoromethyl)phenyl)ethan-1-one

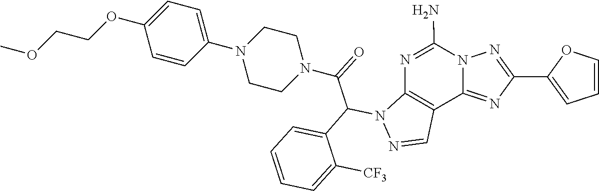 [SMILES: COCCOc1ccc(N2CCN(C(=O)C(c3ccccc3C(F)(F)F)n3ncc4c3nc(N)n3nc(-c5ccco5)nc43)CC2)cc1]

A mixture of 2-(5-amino-2-(furan-2-yl)-7H-pyrazolo[4,3-e][1,2,4]triazolo[1,5-c]pyrimidin-7-yl)-2-(2-(trifluoromethyl)phenyl)acetic acid (50 mg, 0.11 mmol), 1-(4-(2-methoxyethoxy)phenyl)piperazine (32 mg, 0.14 mmol), HATU (53 mg, 0.14 mmol) and DIEA (28 mg, 0.22 mmol) in DMF (5 mL) was stirred at rt for 2 hrs. The solution was added with water (5 mL), extracted with ethyl acetate (10 mL) and washed with brine (10 mL). The organic layer was dried, concentrated and purified by column chromatography (PE:EA=1:1) and preparative TLC (EA) to get the product (10 mg, 14%). ¹H NMR (400 MHz, DMSO-d6) δ 8.29 (s, 1H), 8.21 (br.s, 2H), 7.95 (s, 1H), 7.83 (d, J=8.0 Hz, 1H), 7.70 (t, J=8.0 Hz, 1H), 7.61 (t, J=8.0 Hz, 1H), 7.39 (d, J=8.0 Hz, 1H), 7.24 (d, J=4.0 Hz, 1H), 7.02 (s, 1H), 6.82-6.77 (m, 4H), 6.75-6.73 (m, 1H), 3.98 (t, J=4.0 Hz, 2H), 3.78-3.77 (m, 1H), 3.65-3.51 (m, 3H), 3.32-3.23 (m, 5H), 3.07-3.05 (m, 1H), 2.92-2.90 (m, 1H), 2.69-2.59 (m, 2H) ppm. MS: M/e 662 (M+1)⁺

### Compound A39: 2-(5-amino-2-(furan-2-yl)-7H-pyrazolo[4,3-e][1,2,4]triazolo[1,5-c]pyrimidin-7-yl)-1-(4-(4-(2-(dimethylamino)ethoxy)phenyl)piperazin-1-yl)pentan-1-one

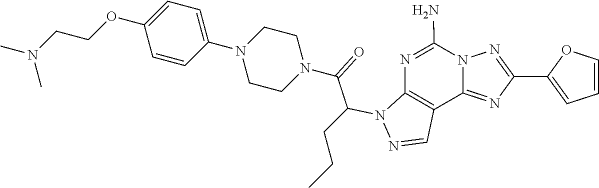 [SMILES: CCCC(C(=O)N1CCN(c2ccc(OCCN(C)C)cc2)CC1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)pentanoic acid (212 mg, 0.62 mmol), N,N-dimethyl-2-(4-(piperazin-1-yl)phenoxy)ethan-1-amine (178 mg, 0.62 mmol), HATU (284 mg, 0.74 mmol) and DIPEA (160 mg, 1.24 mmol) in DMF (5 mL) was stirred overnight. The reaction mixture was poured into H₂O (20 mL) and extracted with EtOAc (20 mL×4). The combined organic layers were washed with brine, dried over Na₂SO₄, concentrated and purified by column chromatography (CH2Cl2/MeOH=10:1, containing 5% NH3. H₂O) to give the target compound (120 mg, 33.8%). ¹H NMR (400 MHz, DMSO-d6) δ 8.20 (s, 1H), 8.16 (s, 2H), 7.95 (s, 1H), 7.23 (d, J=3.2 Hz, 1H), 6.86-6.75 (m, 4H), 6.72 (m, 1H), 5.58 (m, 1H), 3.94 (t, J=5.6 Hz, 2H), 3.69 (m, 1H), 3.51 (m, 3H), 3.29 (s, 1H), 3.05-2.94 (m, 2H), 2.79 (m, 1H), 2.58 (m, 2H), 2.41 (m, 1H), 2.20 (s, 6H), 2.18-1.97 (m, 2H), 1.11 (m, 1H), 0.87 (t, J=7.2 Hz, 3H) ppm. MS: M/e 573 (M+1)⁺.

Compound A39 was separated into two enantiomeric stereoisomers, Compound A39a and Compound A39b (Compound A39a, earlier peak, and Compound A39b, later peak) by chiral prep-HPLC. The chiral separation conditions are shown below.

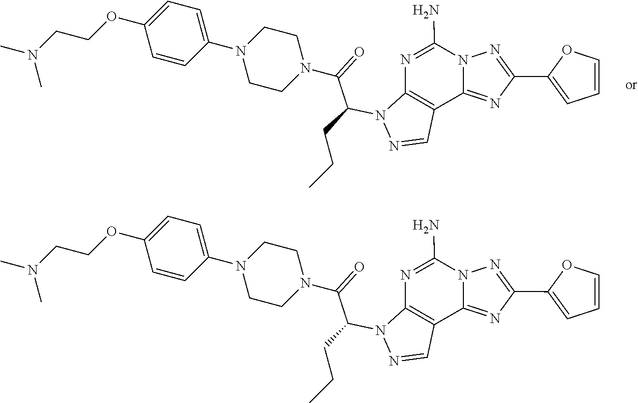 [SMILES: CCC[C@@H](C(=O)N1CCN(c2ccc(OCCN(C)C)cc2)CC1)n1ncc2c1nc(N)n1nc(-c3ccco3)nc21 ; CCC[C@H](C(=O)N1CCN(c2ccc(OCCN(C)C)cc2)CC1)n1ncc2c1nc(N)n1nc(-c3ccco3)nc21]

| Column | CHIRAL ART Cellulose-SB |
|---|---|
| Column size | 2 cm × 25 cm, 5 um |
| Injection | 0.3 mL |
| Mobile phase | Hex(0.1% DEA):EtOH = 50:50 |
| Flow rate | 18 ml/min |
| Wavelength | UV 220 nm |
| Temperature | 25° C. |
| Sample solution | 17.9 mg/ml in DCM:EtOH = 1:3 |
| Prep-HPLC equipment | Prep-Gilson-HPLC |

### Compound A40: 2-(5-amino-2-(furan-2-yl)-7H-pyrazolo[4,3-e][1,2,4]triazolo[1,5-c]pyrimidin-7-yl)-2-(3,4-difluorophenyl)-1-(4-(4-(2-methoxyethoxy)phenyl)piperazin-1-yl)ethan-1-one

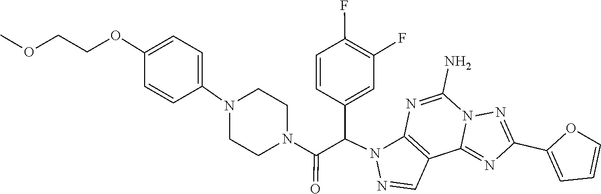 [SMILES: COCCOc1ccc(N2CCN(C(=O)C(c3ccc(F)c(F)c3)n3ncc4c3nc(N)n3nc(-c5ccco5)nc43)CC2)cc1]

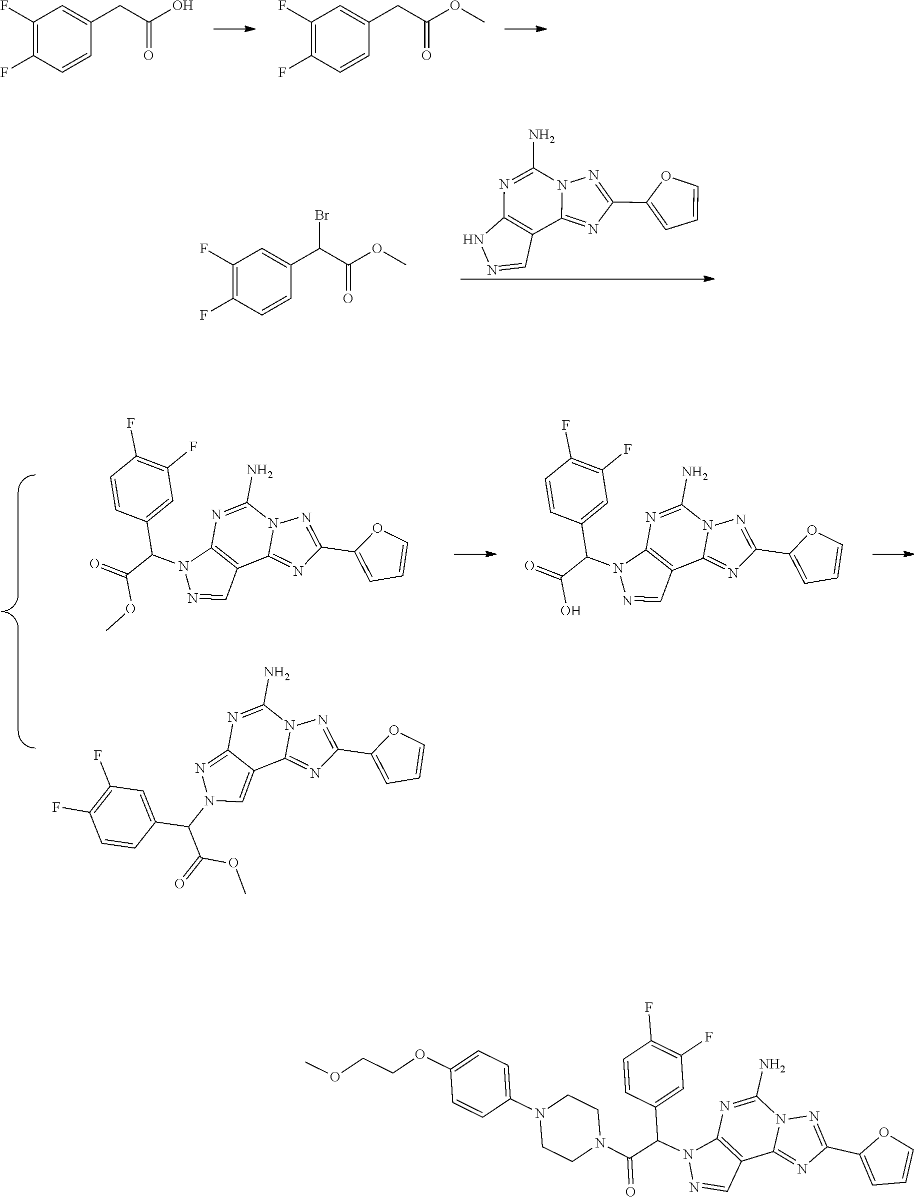

### Step A: methyl 2-(3,4-difluorophenyl)acetate

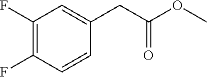

To a stirred solution of 2-(3,4-difluorophenyl)acetic acid (5 g, 29 mmol) in MeOH (50 mL) was added SOCl₂ (3 mL, 116 mmol), the reaction mixture was stirred for 5 hrs at 65° C. Most of solvent was removed and this residue was dissolved by H₂O (50 mL), then adjusted to pH=7˜8 with aq.NaHCO3 and extracted with EtOAc (50 mL×3). The combined organic layers were washed with brine, dried over Na₂SO₄, concentrated to give methyl 2-(3,4-difluorophenyl)acetate (4.5 g, crude) as yellow oil. MS: M/e 1877 (M+1)⁺.

### Step B: methyl 2-bromo-2-(3,4-difluorophenyl)acetate

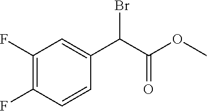

To a stirred mixture of methyl 2-(3,4-difluorophenyl)acetate (4.5 g, crude) in CCl4 (50 mL) was added NBS (6.0 g, 33.7 mmol) and HBr/HOAc (1.0 mL). After the addition, the reaction mixture was stirred for 3 hours at 85° C. The solid of the reaction was filter out, the filtrate was concentrated, and this residue was dissolved by H₂O (50 mL), extracted with EtOAc (50 mL×3), The combined organic layers were washed with brine, dried over Na₂SO₄, concentrated to give methyl 2-bromo-2-(3,4-difluorophenyl)acetate (4.0 g, crud) as a yellow oil. MS: M/e 266 (M+1)⁺

### Step C: methyl 2-(5-amino-2-(furan-2-yl)-7H-pyrazolo[4,3-e][1,2,4]triazolo[1,5-c]pyrimidin-7-yl)-2-(3,4-difluorophenyl)acetate and methyl 2-(5-amino-2-(furan-2-yl)-8H-pyrazolo[4,3-e][1,2,4]triazolo[1,5-c]pyrimidin-8-yl)-2-(3,4-difluorophenyl)acetate

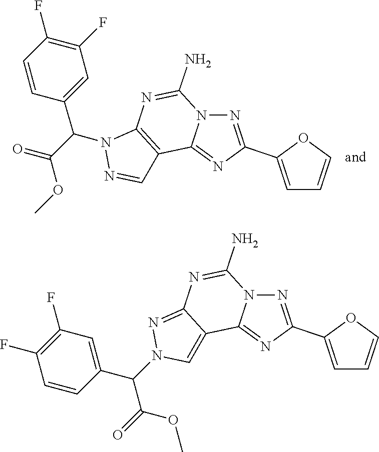 [SMILES: COC(=O)C(c1ccc(F)c(F)c1)n1ncc2c1nc(N)n1nc(-c3ccco3)nc21 ; COC(=O)C(c1ccc(F)c(F)c1)n1cc2c(nc(N)n3nc(-c4ccco4)nc23)n1]

To a stirred solution of 2-(furan-2-yl)-7H-pyrazolo[4,3-e][1,2,4]triazolo[1,5-c]pyrimidin-5-amine (500 mg, 2.07 mmol) in DMF (20 mL) was added K₂CO₃ (750 mg, 5.4 mmol) and methyl 2-bromo-2-(3,4-difluorophenyl)acetate (610 mg, 2.3 mmol). After the addition, the reaction mixture was stirred overnight. The reaction mixture was poured into H₂O (50 mL) and extracted with EtOAc (50 mL×3). The combined organic layers were washed with brine, dried over Na₂SO₄, concentrated and purified by column chromatography (petroleum ether/EtOAc=3:1˜1:1) to give methyl 2-(5-amino-2-(furan-2-yl)-7H-pyrazolo[4,3-e][1,2,4]triazolo[1,5-c]pyrimidin-7-yl)-2-(3,4-difluorophenyl)acetate (200 mg, 45.3%) and methyl 2-(5-amino-2-(furan-2-yl)-8H-pyrazolo[4,3-e][1,2,4]triazolo[1,5-c]pyrimidin-8-yl)-2-(3,4-difluorophenyl)acetate (240 mg, 54.4%) as yellow solids. MS: M/e 426 (M+1)⁺.

### Step D: 2-(5-amino-2-(furan-2-yl)-7H-pyrazolo[4,3-e][1,2,4]triazolo[1,5-c]pyrimidin-7-yl)-2-(3,4-difluorophenyl)acetic Acid

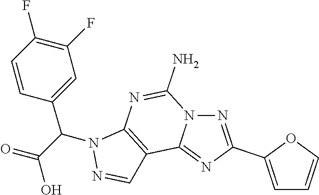 [SMILES: Nc1nc2c(cnn2C(C(=O)O)c2ccc(F)c(F)c2)c2nc(-c3ccco3)nn12]

To a stirred mixture of methyl 2-(5-amino-2-(furan-2-yl)-7H-pyrazolo[4,3-e][1,2,4]triazolo[1,5-c]pyrimidin-7-yl)-2-(3,4-difluorophenyl)acetate (200 mg, 0.47 mmol) in MeOH/H₂O (4.0 mL/1.5 mL) was added aq.NaOH (2.0 M, 1 mL). After the addition, the reaction mixture was stirred for 5 hours at RT. Most of solvent was removed to give the aqueous layer, then acidified to pH=3˜4 with aq.HCl and filtered, the cake was collected, dried to give the target compound (150 mg, 77.7%) as a white solid. MS: M/e 412 (M+1)⁺.

### Step E: 2-(5-amino-2-(furan-2-yl)-7H-pyrazolo[4,3-e][1,2,4]triazolo[1,5-c]pyrimidin-7-yl)-2-(3,4-difluorophenyl)-1-(4-(4-(2-methoxyethoxy)phenyl)piperazin-1-yl)ethan-1-one

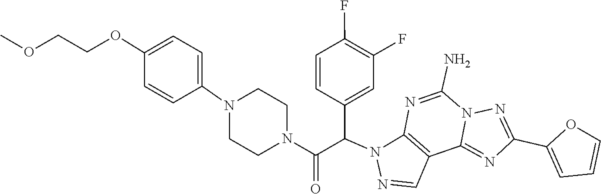 [SMILES: COCCOc1ccc(N2CCN(C(=O)C(c3ccc(F)c(F)c3)n3ncc4c3nc(N)n3nc(-c5ccco5)nc43)CC2)cc1]

A mixture of the product of step D (50 mg, 0.12 mmol), 1-(4-(2-methoxyethoxy)phenyl)piperazine (40 mg, 0.17 mmol), HATU (50 mg, 0.13 mmol) and DIEA (80 mg, 0.23 mmol) in DMF (10 mL) was stirred overnight. The reaction mixture was poured into H₂O (20 mL) and extracted with EtOAc (40 mL×3). The combined organic layers were washed with brine, dried over Na₂SO₄, concentrated and purified by column chromatography (petroleum ether/EtOAc=1:1˜100% EtOAc) to give the target compound (23 mg, 30.4%). ¹H NMR (400 MHz, DMSO-d6) δ 8.26 (s, 1H), 8.18 (br, 2H), 7.95 (s, 1H), 7.52-7.38 (m, 2H), 7.24 (d, J=3.3 Hz, 1H), 7.21 (s, 1H), 6.93 (s, 1H), 6.84-6.76 (m, 4H), 6.74 (dd, J=3.4, 1.8 Hz, 1H), 4.00-3.95 (m, 2H), 3.68-3.57 (m, 4H), 3.48-3.39 (m, 1H), 3.32 (s, 3H), 3.30 (s, 1H), 3.03-2.91 (m, 2H), 2.81-2.70 (m, 1H), 2.62-2.54 (m, 1H) ppm. MS: M/e 630 (M+1)⁺.

### Compound A41: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(3-methoxyphenyl)ethan-1-one

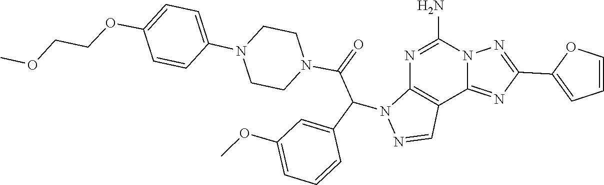 [SMILES: COCCOc1ccc(N2CCN(C(=O)C(c3cccc(OC)c3)n3ncc4c3nc(N)n3nc(-c5ccco5)nc43)CC2)cc1]

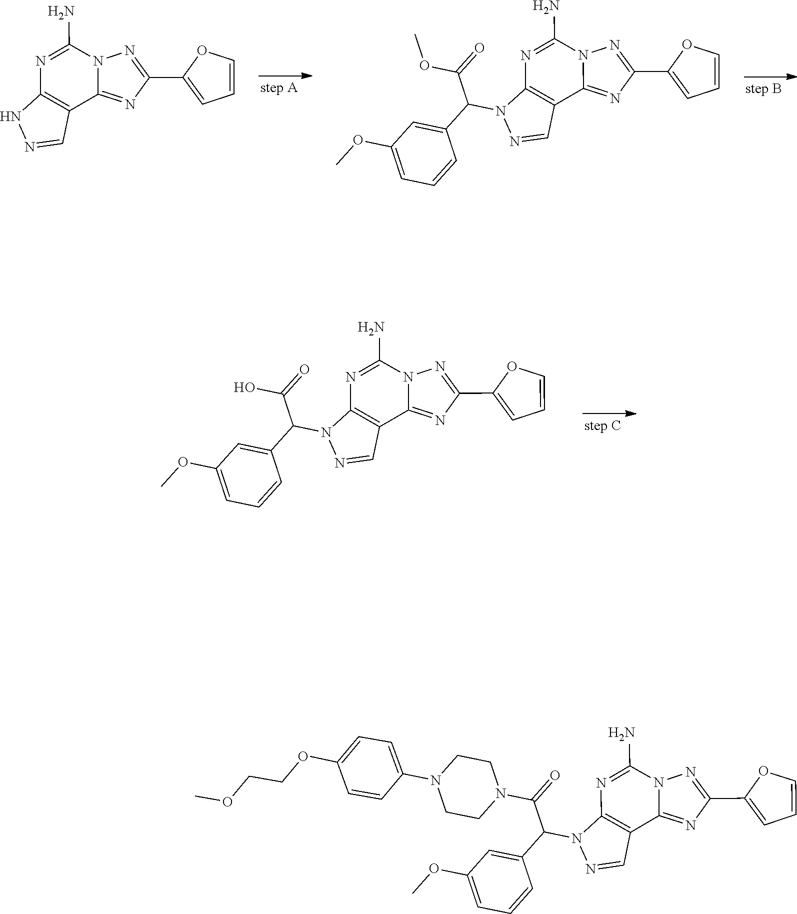 [SMILES: Nc1nc2[nH]ncc2c2nc(-c3ccco3)nn12 ; COC(=O)C(c1cccc(OC)c1)n1ncc2c1nc(N)n1nc(-c3ccco3)nc21 ; COc1cccc(C(C(=O)O)n2ncc3c2nc(N)n2nc(-c4ccco4)nc32)c1 ; COCCOc1ccc(N2CCN(C(=O)C(c3cccc(OC)c3)n3ncc4c3nc(N)n3nc(-c5ccco5)nc43)CC2)cc1]

### Step A: methyl 2-(5-amino-2-(furan-2-yl)-7H-pyrazolo[4,3-e][1,2,4]triazolo[1,5-c]pyrimidin-7-yl)-2-(3-methoxyphenyl)acetate

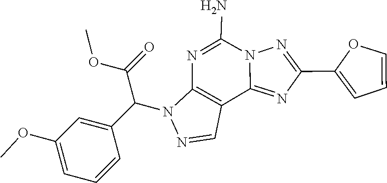 [SMILES: COC(=O)C(c1cccc(OC)c1)n1ncc2c1nc(N)n1nc(-c3ccco3)nc21]

A mixture of 2-(furan-2-yl)-7H-pyrazolo[4,3-e][1,2,4]triazolo[1,5-c]pyrimidin-5-amine (482 mg, 2 mmol) in DMF (10 mL) was added K₂CO₃ (552 mg, 4 mol), then methyl 2-bromo-2-(3-methoxyphenyl)acetate (518 mg, 2 mmol) was added, then the mixture was stirred overnight. The reaction mixture was poured into H₂O (30 mL) and extracted with EtOAc (20 mL×3). The combined organic layers were washed with brine, dried over Na₂SO₄, concentrated and purified by column chromatography (petroleum ether/EtOAc=3:1˜1:1) to give the target compound (186 mg, 22%) as a white solid. MS: M/e 420 (M+1)⁺.

### Step B: 2-(5-amino-2-(furan-2-yl)-7H-pyrazolo[4,3-e][1,2,4]triazolo[1,5-c]pyrimidin-7-yl)-2-(3-methoxyphenyl)acetic Acid

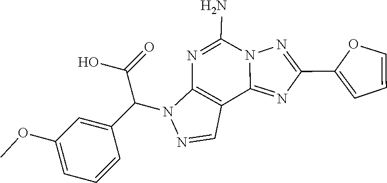 [SMILES: COc1cccc(C(C(=O)O)n2ncc3c2nc(N)n2nc(-c4ccco4)nc32)c1]

To a stirred solution of the product of step A (186 mg, 0.44 mmol) in MeOH (10 mL) was added aq.NaOH (2.0 M, 4 mL). After the addition, the reaction mixture was stirred overnight. Most of MeOH was removed to give the aqueous layer, then acidified to pH=3˜4 with aq.HCl and filtered. The cake was collected, dried to give the target compound (133 mg, 74.6%) as a white solid. MS: M/e 406 (M+1)⁺.

### Step C: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(3-methoxyphenyl)ethan-1-one

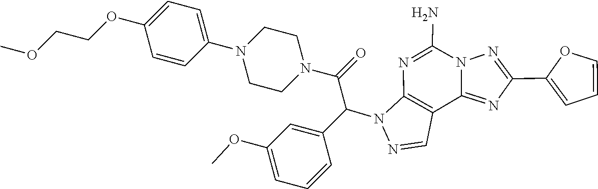 [SMILES: COCCOc1ccc(N2CCN(C(=O)C(c3cccc(OC)c3)n3ncc4c3nc(N)n3nc(-c5ccco5)nc43)CC2)cc1]

A mixture of the product of step B (133 mg, 0.328 mmol), 1-(4-(2-methoxyethoxy)phenyl)piperazine (77.5 mg, 0.328 mmol), HATU (150 mg, 0.393 mmol) and DIEPA (84 mg, 0.626 mmol) in DMF (3 mL) was stirred overnight. The reaction mixture was poured into H₂O (10 mL) and extracted with EtOAc (20 mL×3). The combined organic layers were washed with brine, dried over Na₂SO₄, concentrated and purified by column chromatography (petroleum ether/EtOAc=2:1˜100% EtOAc) to give the target compound (120 mg, 58.7%). ¹H NMR (400 MHz, DMSO-d6) δ 8.21 (s, 1H), 8.16 (s, 2H), 7.95 (m, 1H), 7.30-7.26 (t, J=8.0 Hz, 1H), 7.24 (dd, J=3.2, 0.8 Hz, 1H), 6.97-6.89 (m, 3H), 6.83 (s, 1H), 6.78 (m, 4H), 6.74 (m, 1H), 4.00-3.94 (m, 2H), 3.72 (s, 3H), 3.69-3.63 (m, 2H), 3.62-3.57 (m, 2H), 3.49-3.41 (m, 2H), 3.28 (s, 3H), 3.03-2.87 (m, 2H), 2.68-2.60 (m, 2H) ppm. MS: M/e 624 (M+1)⁺.

### Compound A42: 2-(5-amino-2-(furan-2-yl)-7H-pyrazolo[4,3-e][1,2,4]triazolo[1,5-c]pyrimidin-7-yl)-2-(3-fluorophenyl)-1-(4-(4-(2-methoxyethoxy)phenyl)piperazin-1-yl)ethan-1-one

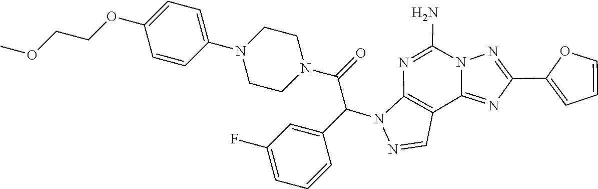 [SMILES: COCCOc1ccc(N2CCN(C(=O)C(c3cccc(F)c3)n3ncc4c3nc(N)n3nc(-c5ccco5)nc43)CC2)cc1]

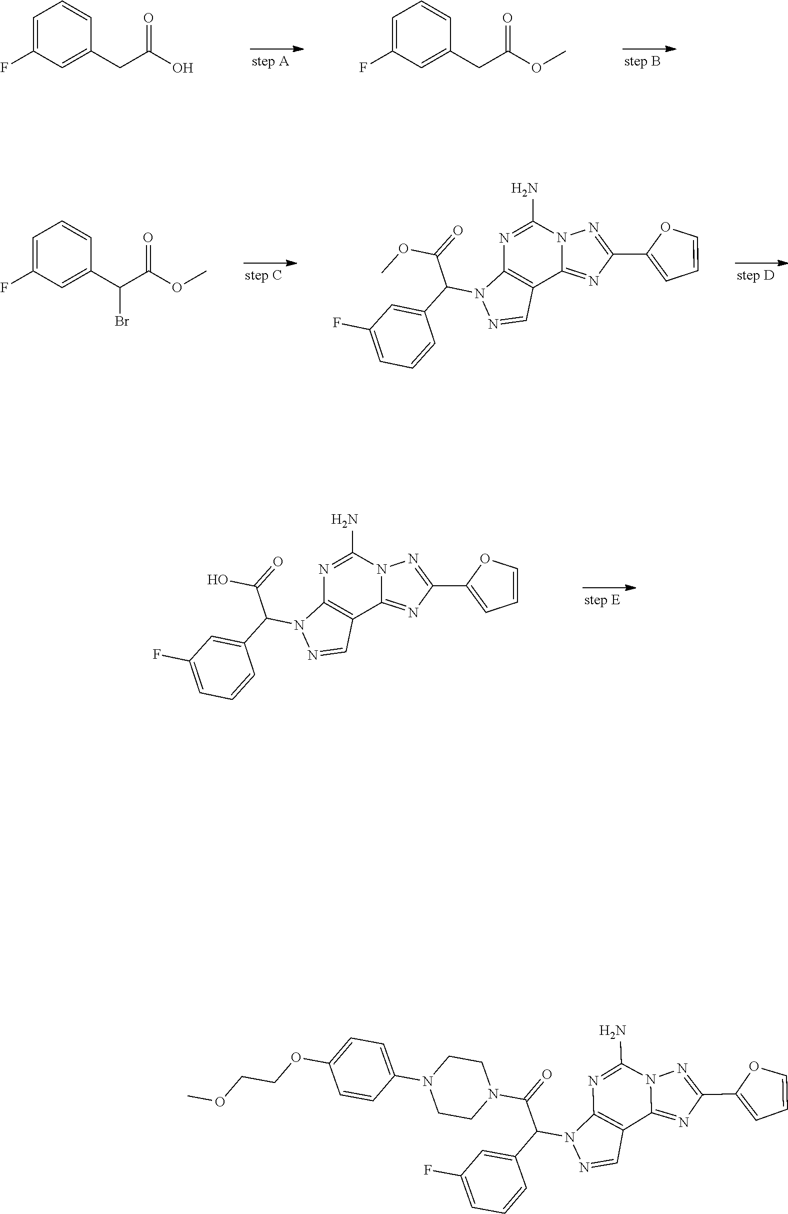 [SMILES: O=C(O)Cc1cccc(F)c1 ; COC(=O)Cc1cccc(F)c1 ; COC(=O)C(Br)c1cccc(F)c1 ; COC(=O)C(c1cccc(F)c1)n1ncc2c1nc(N)n1nc(-c3ccco3)nc21 ; Nc1nc2c(cnn2C(C(=O)O)c2cccc(F)c2)c2nc(-c3ccco3)nn12 ; COCCOc1ccc(N2CCN(C(=O)C(c3cccc(F)c3)n3ncc4c3nc(N)n3nc(-c5ccco5)nc43)CC2)cc1]

### Step A: methyl 2-(3-fluorophenyl)acetate

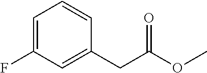

To a stirred mixture of 2-(3-fluorophenyl)acetic acid (1.54 g, 10 mmol) in MeOH (20 mL) was added SOCl₂ (2.38 g, 20 mmol) dropwise at 0° C. After the addition, the reaction was stirred overnight. Most of the solvent was removed to give the residue, which was dissolved in EtOAc (30 mL) and washed with aq.K₂CO₃, brine, dried over Na₂SO₄, and concentrated to give the target compound (1.51 g, 90%) as colorless oil.

### Step B: methyl 2-bromo-2-(3-fluorophenyl)acetate

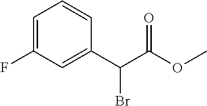

To a stirred solution of the product of step A (1 g, 5.95 mmol) in CCl₄ (10 mL) was added NBS (1.16 g, 6.54 mmol), then aq.HBr (40%, 2 drops) was added. After the addition, the reaction mixture was refluxed for 3 days. The reaction mixture was cooled to room temperature and filtered and the filtrate was collected to give the target compound (1.4 g, 99%) as colorless oil.

### Step C: methyl 2-(5-amino-2-(furan-2-yl)-7H-pyrazolo[4,3-e][1,2,4]triazolo[1,5-c]pyrimidin-7-yl)-2-(3-fluorophenyl)acetate

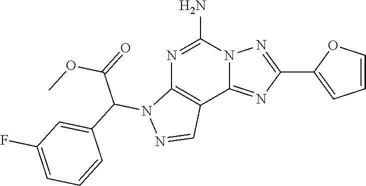 [SMILES: COC(=O)C(c1cccc(F)c1)n1ncc2c1nc(N)n1nc(-c3ccco3)nc21]

To a stirred solution of 2-(furan-2-yl)-7H-pyrazolo[4,3-e][1,2,4]triazolo[1,5-c]pyrimidin-5-amine (482 mg, 2 mmol) in DMF (5 mL) was added K₂CO₃ (552 mg, 4 mmol), then the product of step B (494 mg, 2 mmol) was added. After the addition, the reaction mixture was stirred overnight. The reaction mixture was poured into H₂O (20 mL) and extracted with EtOAc (10 mL×3). The combined organic layers were washed with brine, dried over Na₂SO₄, concentrated and purified by column chromatography (petroleum ether/EtOAc=3:1˜1:1) to give the target compound (140 mg, 17.2%) as a white solid. MS: M/e 408 (M+1)⁺.

### Step D: 2-(5-amino-2-(furan-2-yl)-7H-pyrazolo[4,3-e][1,2,4]triazolo[1,5-c]pyrimidin-7-yl)-2-(3-fluorophenyl)acetic Acid

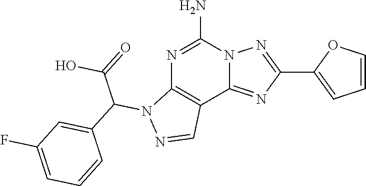 [SMILES: Nc1nc2c(cnn2C(C(=O)O)c2cccc(F)c2)c2nc(-c3ccco3)nn12]

To a stirred solution of the product of step C (140 mg, 0.18 mmol) in MeOH (10 mL) was added aq.NaOH (2.0 M, 3 mL). After the addition, the reaction mixture was stirred overnight. Most of MeOH was removed to give aqueous layer, then acidified to pH=3˜4 and filtered. The cake was collected, dried to give the target compound (100 mg, 74%) as a white solid. MS: M/e 394 (M+1)⁺.

### Step E: 2-(5-amino-2-(furan-2-yl)-7H-pyrazolo[4,3-e][1,2,4]triazolo[1,5-c]pyrimidin-7-yl)-2-(3-fluorophenyl)-1-(4-(4-(2-methoxyethoxy)phenyl)piperazin-1-yl)ethan-1-one

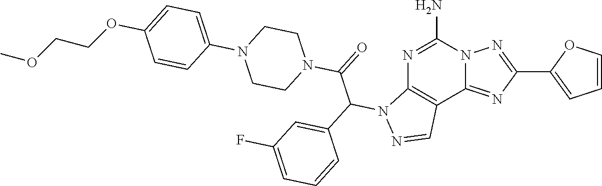 [SMILES: COCCOc1ccc(N2CCN(C(=O)C(c3cccc(F)c3)n3ncc4c3nc(N)n3nc(-c5ccco5)nc43)CC2)cc1]

A mixture of the product of step D (46 mg, 0.117 mmol), 1-(4-(2-methoxyethoxy)phenyl)piperazine (27.6 mg, 0.17 mmol), HATU (53.6 mg, 0.14 mmol) and DIPEA (30 mg, 0.234 mmol) in DMF (4 mL) was stirred overnight. The reaction mixture was poured into H₂O (20 mL), and extracted with EtOAc (20 mL×3). The combined organic layers were washed with brine, dried over Na₂SO₄, concentrated and purified by column chromatography (petroleum ether/EtOAc=2:1˜100% EtOAc) to give the target compound (32 mg, 44.7%). ¹H NMR (400 MHz, DMSO-d6) δ 8.26 (s, 1H), 8.20 (s, 2H), 7.97-7.94 (m, 1H), 7.44-7.38 (m, 1H), 7.27-7.14 (m, 4H), 6.92 (s, 1H), 6.79 (s, 4H), 6.74 (m, 1H), 4.00-3.95 (m, 2H), 3.65 (m, 2H), 3.62-3.57 (m, 2H), 3.42 (m, 1H), 3.31 (m, 1H), 3.28 (s, 3H), 2.96 (m, 2H), 2.69 (m, 2H), 2.55 (m, 1H) ppm. MS: M/e 612 (M+1)⁺.

### Compound A43: 2-(5-amino-2-(furan-2-yl)-7H-pyrazolo[4,3-e][1,2,4]triazolo[1,5-c]pyrimidin-7-yl)-2-(2-fluorophenyl)-1-(4-(4-(2-methoxyethoxy)phenyl)piperazin-1-yl)ethan-1-one

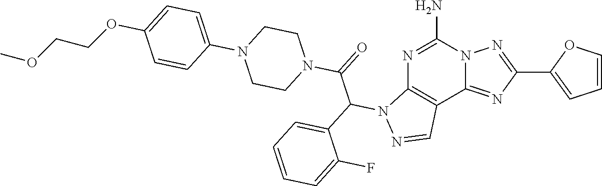 [SMILES: COCCOc1ccc(N2CCN(C(=O)C(c3ccccc3F)n3ncc4c3nc(N)n3nc(-c5ccco5)nc43)CC2)cc1]

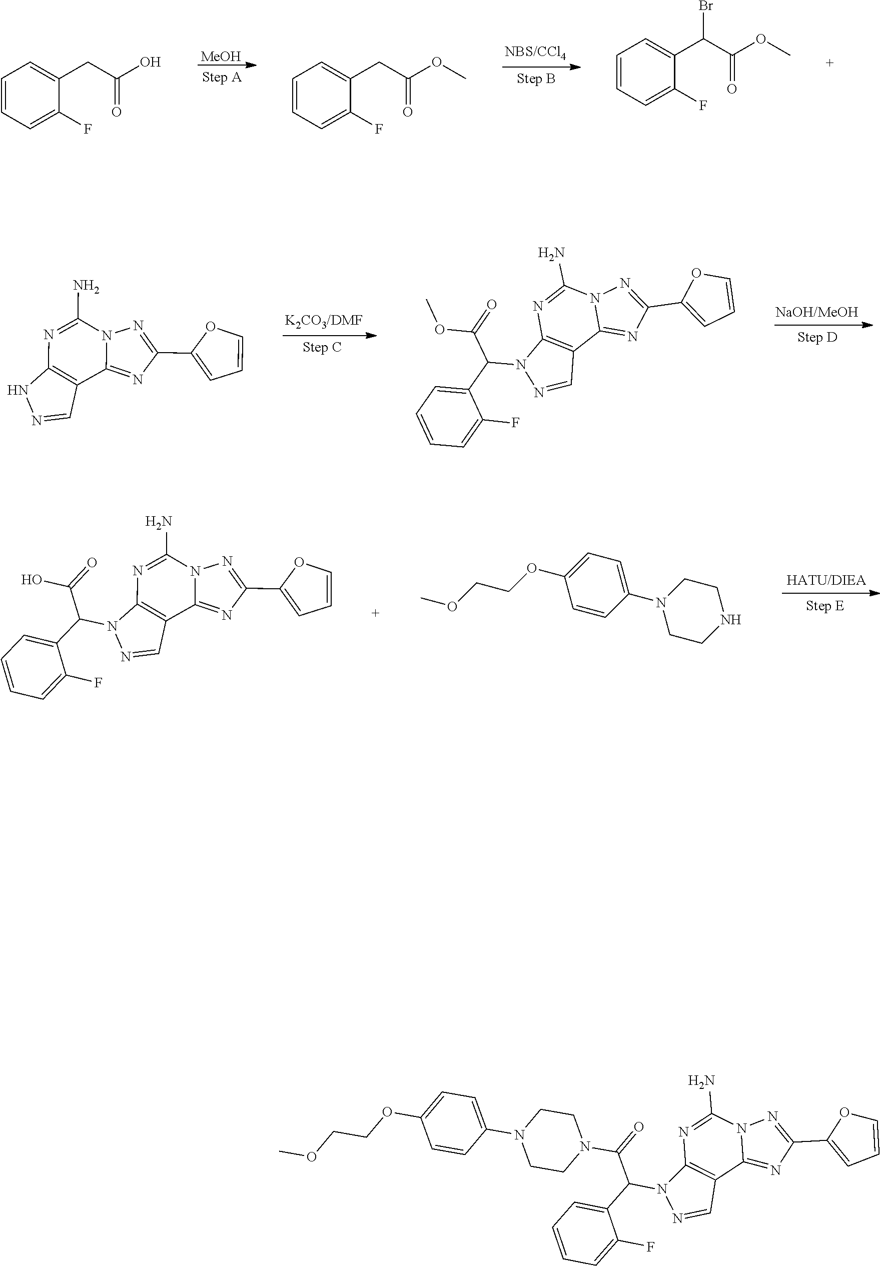

### Step A: methyl 2-(2-fluorophenyl)acetate

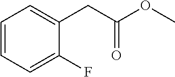

Thionyl chloride (5 mL) was added dropwise to a solution of 2-(2-fluorophenyl)acetic acid (5 g, 32.5 mmol) in methanol (20 mL) at room temperature. After addition, the mixture was stirred overnight. The solvent was evaporated under reduced pressure and the residue was used in the next step directly (5.4 g, 100%) MS: M/e 169 (M+1)⁺

### Step B: methyl 2-bromo-2-(2-fluorophenyl)acetate

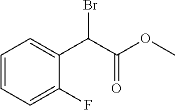

A mixture of methyl 2-(2-fluorophenyl)acetate (5.4 g, 32.5 mmol), N-bromosuccinimide (6.4 g, 36 mmol) and AIBN (267 mg, 1.6 mmol) in CCl₄ (50 mL) was heated at 80° C. for 3 hrs. The solution was concentrated and purified by column chromatography (PE:EA=50:1) to get the product (7.1 g, 92%). ¹H NMR (400 MHz, DMSO-d6) δ 7.60-6.43 (m, 2H), 7.27-7.24 (m, 2H), 6.18 (s, 1H), 3.74 (s, 3H) ppm. MS: M/e 247 (M+1)⁺

### Step C: methyl 2-(5-amino-2-(furan-2-yl)-7H-pyrazolo[4,3-e][1,2,4]triazolo[1,5-c]pyrimidin-7-yl)-2-(2-fluorophenyl)acetate

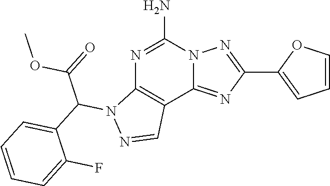 [SMILES: COC(=O)C(c1ccccc1F)n1ncc2c1nc(N)n1nc(-c3ccco3)nc21]

A mixture of 2-(furan-2-yl)-7H-pyrazolo[4,3-e][1,2,4]triazolo[1,5-c]pyrimidin-5-amine (3 g, 12.4 mmol), methyl 2-bromo-2-(2-fluorophenyl)acetate (3.6 g, 14.9 mmol) and K₂CO₃ (3.4 g, 24.8 mmol) in DMF (30 mL) was stirred at rt overnight. The solution was added with water (30 mL), extracted with ethyl aceate (30 mL) and washed with brine (30 mL). The organic layer was dried over Na₂SO₄, concentrated and purified by column chromatography (PE:EA=2:1) to get methyl 2-(5-amino-2-(furan-2-yl)-7H-pyrazolo[4,3-e][1,2,4]triazolo[1,5-c]pyrimidin-7-yl)-2-(2-fluorophenyl)acetate (1.05 g, 21%). MS: M/e 408 (M+1)⁺

### Step D: 2-(5-amino-2-(furan-2-yl)-7H-pyrazolo[4,3-e][1,2,4]triazolo[1,5-c]pyrimidin-7-yl)-2-(2-fluorophenyl)acetic Acid

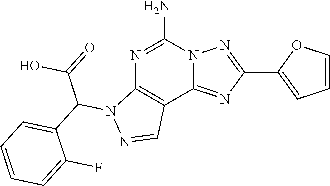 [SMILES: Nc1nc2c(cnn2C(C(=O)O)c2ccccc2F)c2nc(-c3ccco3)nn12]

NaOH solution (400 mg, in 5 mL of water) was added to a solution of methyl 2-(5-amino-2-(furan-2-yl)-7H-pyrazolo[4,3-e][1,2,4]triazolo[1,5-c]pyrimidin-7-yl)-2-(2-fluorophenyl)acetate (1.05 g, 2.6 mmol) in methanol (15 mL). The reaction mixture was stirred at rt overnight. The solution was concentrated, added with water (10 mL) and acidified with 1N HCl solution to pH=5. The precipitated solid was filtered and dried to get the desired product as a white solid (930 mg, 93%). MS: M/e 394 (M+1)⁺

### Step E: 2-(5-amino-2-(furan-2-yl)-7H-pyrazolo[4,3-e][1,2,4]triazolo[1,5-c]pyrimidin-7-yl)-2-(2-fluorophenyl)-1-(4-(4-(2-methoxyethoxy)phenyl)piperazin-1-yl)ethan-1-one

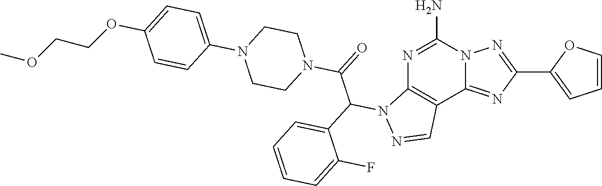 [SMILES: COCCOc1ccc(N2CCN(C(=O)C(c3ccccc3F)n3ncc4c3nc(N)n3nc(-c5ccco5)nc43)CC2)cc1]

A mixture of 2-(5-amino-2-(furan-2-yl)-7H-pyrazolo[4,3-e][1,2,4]triazolo[1,5-c]pyrimidin-7-yl)-2-(2-fluorophenyl)acetic acid (930 mg, 2.4 mmol), 1-(4-(2-methoxyethoxy)phenyl)piperazine (835 mg, 3.5 mmol), HATU (1.1 g, 2.8 mmol) and DIEA (609 mg, 4.7 mmol) in DMF (50 mL) was stirred at rt for 2 hrs. The solution was added with water (30 mL), extracted with ethyl acetate (50 mL) and washed with brine (50 mL). The organic layer was dried, concentrated and purified by column chromatography (PE:EA=1:1) to get the product (890 mg, 62%). ¹H NMR (400 MHz, DMSO-d6) δ 8.29 (s, 1H), 8.25 (br.s, 2H), 7.96 (s, 1H), 7.45-7.41 (m, 1H), 7.23-7.12 (m, 4H), 6.97 (s, 1H), 6.82-6.77 (m, 4H), 6.75-6.73 (m, 1H), 3.98 (t, J=4.0 Hz, 2H), 3.73-3.59 (m, 4H), 3.92-3.89 (m, 2H), 3.28 (s, 3H), 2.98 (br.s, 2H), 2.66-2.62 (m, 2H) ppm. MS: M/e 612 (M+1)⁺

Compound A43 was separated into two enantiomeric stereoisomers (Compound A43a, earlier peak, and Compound A43b, later peak) by chiral prep-HPLC. The chiral separation conditions are shown below.

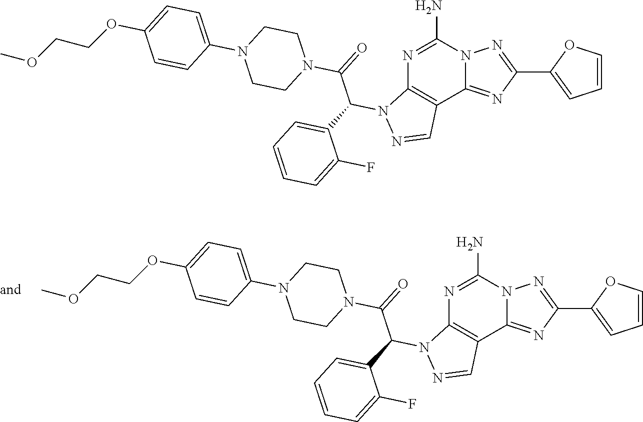 [SMILES: COCCOc1ccc(N2CCN(C(=O)[C@@H](c3ccccc3F)n3ncc4c3nc(N)n3nc(-c5ccco5)nc43)CC2)cc1 ; COCCOc1ccc(N2CCN(C(=O)[C@H](c3ccccc3F)n3ncc4c3nc(N)n3nc(-c5ccco5)nc43)CC2)cc1]

| Column | Chiralpak IG |
|---|---|
| Column size | 2 cm × 25 cm, 5 um |
| Injection | 1.5 mL |
| Mobile phase | (Hex:DCM = 2:1):EtOH = 50:50 |
| Flow rate | 20 mg/min |
| Wavelength | UV 220 nm |
| Temperature | 25° C. |
| Sample solution | 25 mg/ml in MeOH:DCM = 1:1 |
| Prep-HPLC equipment | Prep-HPLC-04 |

### Compound A44: 2-(5-amino-2-(furan-2-yl)-7H-pyrazolo[4,3-e][1,2,4]triazolo[1,5-c]pyrimidin-7-yl)-1-(4-(2-fluoro-4-(2-methoxyethoxy)phenyl)piperazin-1-yl)-2-phenylethan-1-one

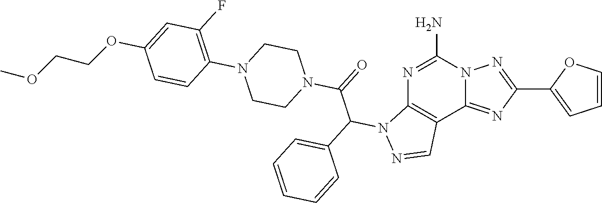 [SMILES: COCCOc1ccc(N2CCN(C(=O)C(c3ccccc3)n3ncc4c3nc(N)n3nc(-c5ccco5)nc43)CC2)c(F)c1]

A mixture of 2-(5-amino-2-(furan-2-yl)-7H-pyrazolo[4,3-e][1,2,4]triazolo[1,5-c]pyrimidin-7-yl)-2-phenylacetic acid (37.5 mg, 0.1 mmol), 1-(2-fluoro-4-(2-methoxyethoxy)phenyl)piperazine hydrochloride (29 mg, 0.1 mmol) and HATU (46 mg, 0.12 mmol) in DMF (2 mL) was stirred overnight. The mixture was poured into H₂O (15 mL) and extracted with EtOAc (10 mL×3). The combined organic layers were washed with brine, dried over Na₂SO₄, concentrated and purified by column chromatography (petroleum ether/EtOAc=2:1˜100% EtOAc) to give the target compound (34 mg, 55.6%). ¹H NMR (400 MHz, DMSO-d6) δ 8.21 (s, 1H), 8.15 (s, 2H), 7.95 (s, 1H), 7.39-7.34 (m, 5H), 7.24 (d, J=3.2 Hz, 1H), 6.90-6.63 (m, 5H), 4.05-3.97 (m, 2H), 3.68-3.59 (m, 4H), 3.45-3.35 (m, 2H), 3.28 (s, 3H), 2.89-2.85 (m, 2H) ppm. MS: M/e 612 (M+1)⁺.

Compound A44 was separated into two enantiomeric stereoisomers, Compound A44a, Compound A44b (Compound A44a, earlier peak, and Compound A44b, later peak) by chiral prep-HPLC. The chiral separation conditions are shown below.

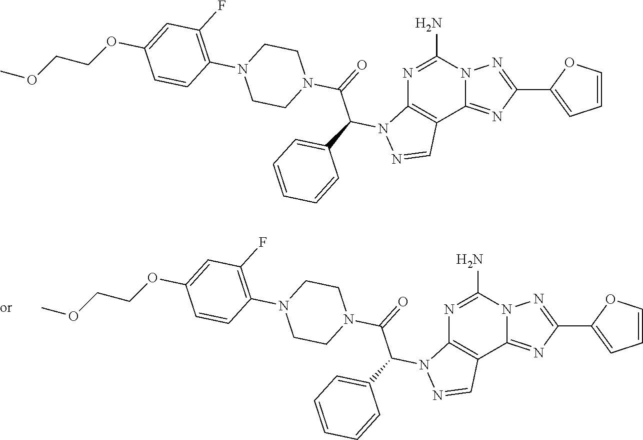 [SMILES: COCCOc1ccc(N2CCN(C(=O)[C@H](c3ccccc3)n3ncc4c3nc(N)n3nc(-c5ccco5)nc43)CC2)c(F)c1 ; COCCOc1ccc(N2CCN(C(=O)[C@@H](c3ccccc3)n3ncc4c3nc(N)n3nc(-c5ccco5)nc43)CC2)c(F)c1]

| Column | Chiralpak IA |
|---|---|
| Column size | 2 cm × 25 cm, 5 um |
| Injection | 2 mL |
| Mobile phase | (Hex:DCM = 3:1):EtOH = 50:50 |
| Flow rate | 17 ml/min |
| Wavelength | UV 220 nm |
| Temperature | 25° C. |
| Sample solution | 9.7 mg/ml in DCM:MeOH = 1:3 |
| Prep-HPLC equipment | Prep-Gilson-HPLC |

### Compound A45: 2-(5-amino-2-(furan-2-yl)-7H-pyrazolo[4,3-e][1,2,4]triazolo[1,5-c]pyrimidin-7-yl)-1-(4-(4-fluorophenyl)piperazin-1-yl)pentan-1-one

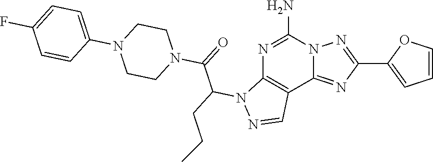 [SMILES: CCCC(C(=O)N1CCN(c2ccc(F)cc2)CC1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)pentanoic acid (60 mg, 0.18 mmol), 1-(4-fluorophenyl)piperazine hydrochloride (42 mg, 0.19 mmol), HATU (82 mg, 0.22 mmol) and DIEA (47 mg, 0.36 mmol) in DMF (5 mL) was stirred at rt for 2 hrs. The solution was added with water (10 mL), extracted with ethyl acetate (10 mL) and washed with brine (10 mL). The organic layer was dried, concentrated and purified by column chromatography (PE:EA=1:1) to get the desired product (58 mg, 66%). ¹H NMR (400 MHz, DMSO-d6) δ 8.21 (s, 1H), 8.18 (br.s, 2H), 7.95 (s, 1H), 7.24 (d, J=4.0 Hz, 1H), 7.01-6.73 (m, 5H), 5.60 (dd, J=8.0 Hz, 4.0 Hz, 1H), 3.71-3.49 (m, 4H), 3.09-2.86 (m, 3H), 2.48 (br.s, 1H), 2.16-1.99 (m, 2H), 1.28-1.20 (m, 2H), 1.10-0.86 (m, 3H) ppm. MS: M/e 504 (M+1)⁺

### Compound A46: 2-(5-amino-2-(furan-2-yl)-7H-pyrazolo[4,3-e][1,2,4]triazolo[1,5-c]pyrimidin-7-yl)-1-((S)-4-(4-(2-methoxyethoxy)phenyl)-3-methylpiperazin-1-yl)-2-phenylethan-1-one

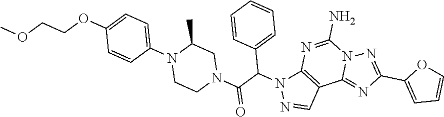 [SMILES: COCCOc1ccc(N2CCN(C(=O)C(c3ccccc3)n3ncc4c3nc(N)n3nc(-c5ccco5)nc43)C[C@@H]2C)cc1]

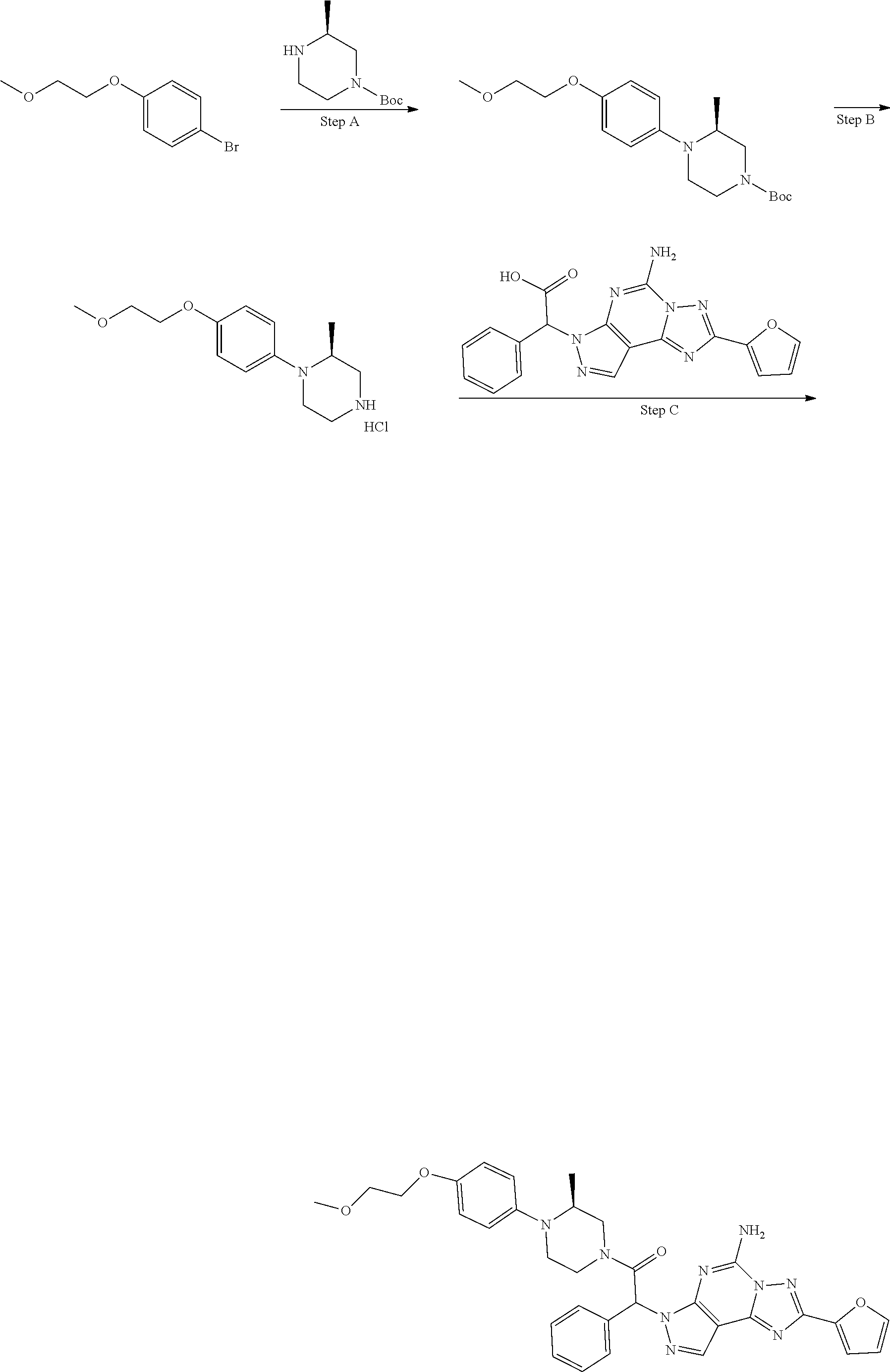

### Step A: tert-butyl (S)-4-(4-(2-methoxyethoxy)phenyl)-3-methylpiperazine-1-carboxylate

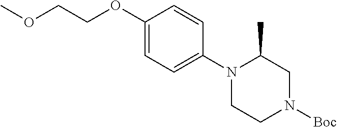 [SMILES: COCCOc1ccc(N2CCN(C(=O)OC(C)(C)C)C[C@@H]2C)cc1]

A mixture of 1-bromo-4-(2-methoxyethoxy)benzene (1.84 g, 8 mmol), tert-butyl (S)-3-methylpiperazine-1-carboxylate (1.76 g, 8.8 mmol), Pd₂(dba)₃ (732 mg, 0.8 mmol), X-phos (761 mg, 1.6 mmol) and Cs₂CO₃ (5.2 g, 16 mmol) in toluene (50 mL) was stirred at 90° C. under N₂ for 16 hrs. The mixture was diluted with EA (20 mL) and the resulting suspension was filtered. The filtrate was concentrated and the residue was purified by column chromatography (eluting with EA:PE=1: 8) to give the title product (1.8 g, crude) as a light yellow oil. MS: M/e 351 (M+1)⁺.

### Step B: (S)-1-(4-(2-methoxyethoxy)phenyl)-2-methylpiperazine Hydrochloride

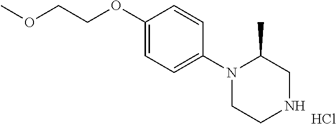 [SMILES: COCCOc1ccc(N2CCNC[C@@H]2C)cc1 ; Cl]

To a solution of tert-butyl (S)-4-(4-(2-methoxyethoxy)phenyl)-3-methylpiperazine-1-carboxylate (1.8 g, 5.12 mmol) in CH₂Cl₂ (30 mL) was added 2M HCl in dioxane (15 mL) at rt and the resulting solution was stirred at rt for 16 hrs. The reaction was quenched with water (50 mL), extracted with CH₂Cl₂ (40 mL×3). The aqueous layer was collected and concentrated to give the title product (1.2 g) as light yellow foam which was used for the next step directly. MS: M/e 251 (M+1)⁺.

### Step C: 2-(5-amino-2-(furan-2-yl)-7H-pyrazolo[4,3-e][1,2,4]triazolo[1,5-c]pyrimidin-7-yl)-1-((S)-4-(4-(2-methoxyethoxy)phenyl)-3-methylpiperazin-1-yl)-2-phenylethan-1-one

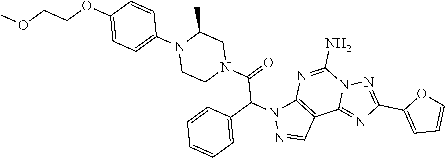 [SMILES: COCCOc1ccc(N2CCN(C(=O)C(c3ccccc3)n3ncc4c3nc(N)n3nc(-c5ccco5)nc43)C[C@@H]2C)cc1]

To a mixture of (S)-1-(4-(2-methoxyethoxy)phenyl)-2-methylpiperazine (388 mg, 1.2 mmol), 2-(5-amino-2-(furan-2-yl)-7H-pyrazolo[4,3-e][1,2,4]triazolo[1,5-c]pyrimidin-7-yl)-2-phenylacetic acid (412 mg, 1.1 mmol), DIPEA (425 mg, 3.3 mmol) in DMF (5 mL) was added HATU (420 mg, 1.1 mmol) at rt and the mixture was stirred at rt for 16 hrs. The reaction was quenched with water (40 mL) to form a suspension. The suspension was filtered and the solid was purified by column chromatography (DCM:MeOH=50:1) to give the title product (330 mg, yield: 49%). ¹H NMR (400 MHz, DMSO-d6) δ 8.36-8.10 (m, 3H), 7.95 (s, 1H), 7.55-7.30 (m, 5H), 7.24 (d, J=2.0 Hz, 1H), 6.95-6.70 (m, 6H), 4.04-3.95 (m, 2H), 3.93-3.76 (m, 1H), 3.76-3.64 (m, 1H), 3.63-3.56 (m, 2H), 3.53-3.45 (m, 1H), 3.28 (s, 3H), 3.24-3.12 (m, 1H), 3.00-2.65 (m, 2H), 2.41-2.29 (m, 1H), 0.91-0.70 (m, 2H), 0.55-0.42 (m, 1H). MS: M/e 608 (M+1)⁺.

Compound A46 was separated into two enantiomeric stereoisomers, Compound A46a (fast isomer), and Compound A46b (slow isomer) by chiral prep-HPLC. The chiral separation conditions are shown below.

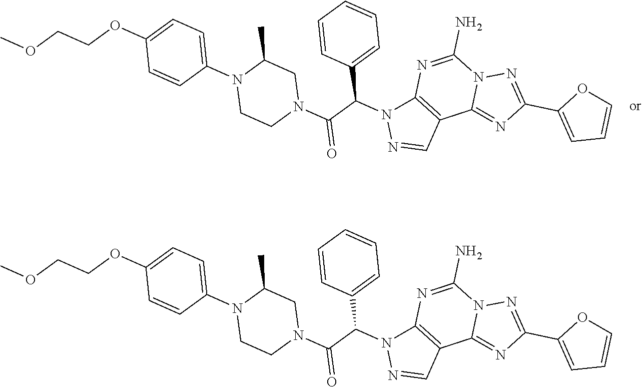 [SMILES: COCCOc1ccc(N2CCN(C(=O)[C@@H](c3ccccc3)n3ncc4c3nc(N)n3nc(-c5ccco5)nc43)C[C@@H]2C)cc1 ; COCCOc1ccc(N2CCN(C(=O)[C@H](c3ccccc3)n3ncc4c3nc(N)n3nc(-c5ccco5)nc43)C[C@@H]2C)cc1]

| Column | CHIRAL ART Cellulose-SB |
|---|---|
| Column size | 2 cm × 25 cm, 5 um |
| Injection | 0.4 mL |
| Mobile phase | (Hex:DCM = 3:1):EtOH = 50:50 |
| Flow rate | 20 ml/min |
| Wavelength | UV 220 nm |
| Temperature | 25° C. |
| Sample solution | 15.6 mg/ml in EtOH:DCM = 3:1 |
| Prep-HPLC equipment | Prep-Gilson-HPLC |

### Compound A47: 2-(5-amino-2-(furan-2-yl)-7H-pyrazolo[4,3-e][1,2,4]triazolo[1,5-c]pyrimidin-7-yl)-1-((R)-4-(4-(2-methoxyethoxy)phenyl)-3-methylpiperazin-1-yl)-2-phenylethan-1-one

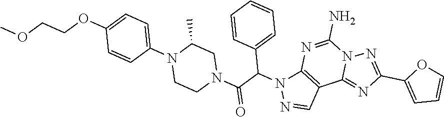 [SMILES: COCCOc1ccc(N2CCN(C(=O)C(c3ccccc3)n3ncc4c3nc(N)n3nc(-c5ccco5)nc43)C[C@H]2C)cc1]

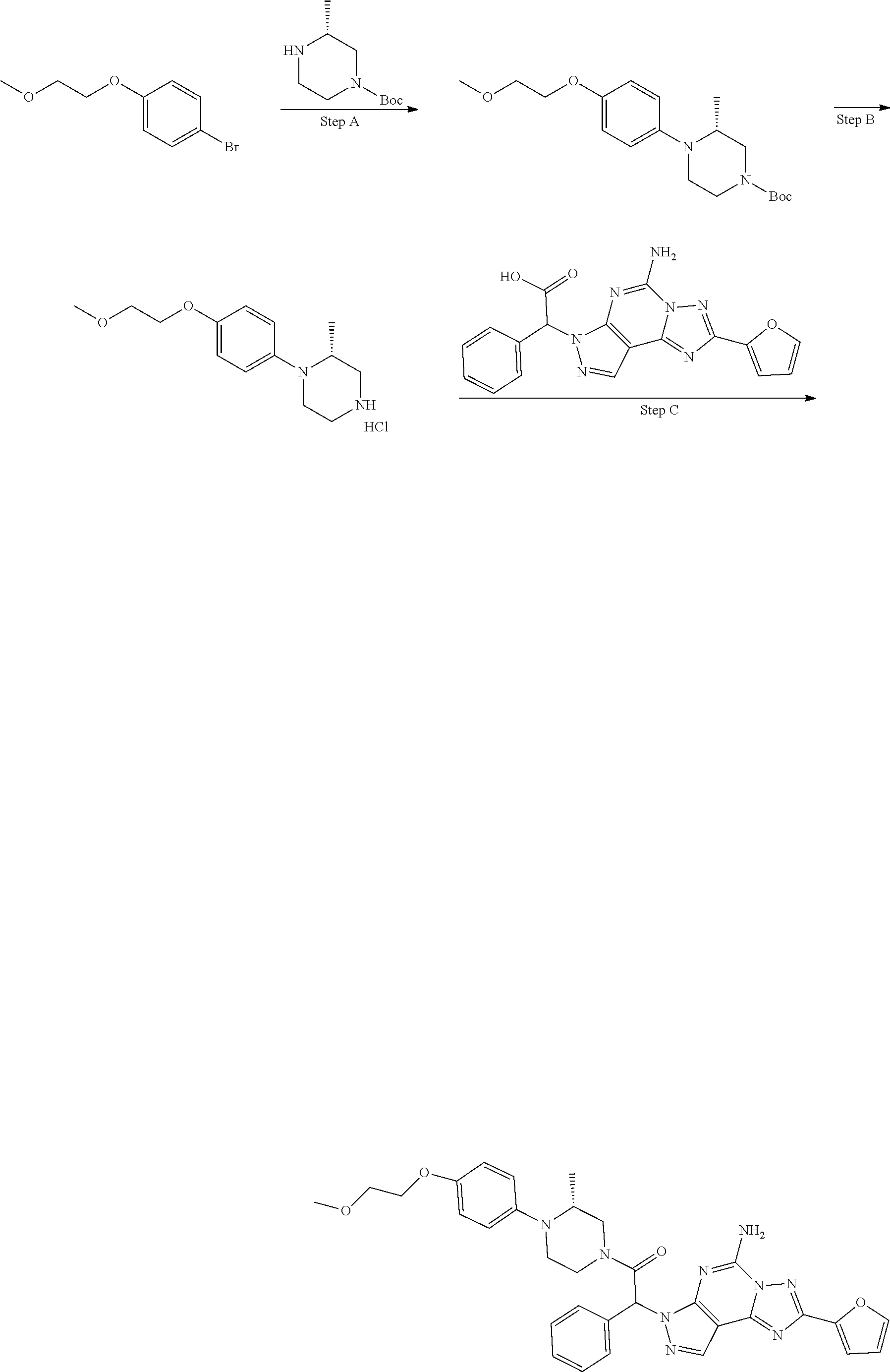

### Step A: tert-butyl (R)-4-(4-(2-methoxyethoxy)phenyl)-3-methylpiperazine-1-carboxylate

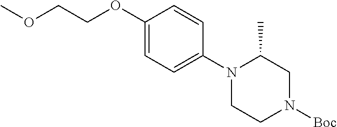 [SMILES: COCCOc1ccc(N2CCN(C(=O)OC(C)(C)C)C[C@H]2C)cc1]

A mixture of 1-bromo-4-(2-methoxyethoxy)benzene (1.84 g, 8 mmol), tert-butyl (R)-3-methylpiperazine-1-carboxylate (1.76 g, 8.8 mmol), Pd₂(dba)₃ (732 mg, 0.8 mmol), X-phos (761 mg, 1.6 mmol) and Cs₂CO₃ (5.2 g, 16 mmol) in toluene (50 mL) was stirred at 100° C. under N₂ for 16 hrs. The mixture was diluted with EA (20 mL) and the resulting suspension was filtered. The filtrate was concentrated and purified by column chromatography (eluting with EA:PE=1:8) to give the title product (2.3 g, crude) as a light yellow oil. MS: M/e 351 (M+1)⁺.

### Step B: (R)-1-(4-(2-methoxyethoxy)phenyl)-2-methylpiperazine Hydrochloride

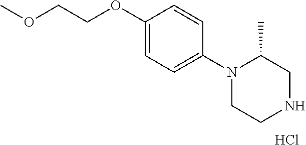 [SMILES: COCCOc1ccc(N2CCNC[C@H]2C)cc1 ; Cl]

To a solution of tert-butyl (R)-4-(4-(2-methoxyethoxy)phenyl)-3-methylpiperazine-1-carboxylate (2.3 g, crude) in CH₂Cl₂ (40 mL) was added 2M HCl in dioxane (20 mL) at rt and the resulting solution was stirred at rt for 16 hrs. The reaction was quenched with water (60 mL), extracted with CH₂Cl₂ (30 mL×3). The aqueous layer was collected and concentrated to give the title product (1.8 g) as HCl salt which was used for the next step directly. MS: M/e 251 (M+1)⁺.

### Step C: 2-(5-amino-2-(furan-2-yl)-7H-pyrazolo[4,3-e][1,2,4]triazolo[1,5-c]pyrimidin-7-yl)-1-((R)-4-(4-(2-methoxyethoxy)phenyl)-3-methylpiperazin-1-yl)-2-phenylethan-1-one

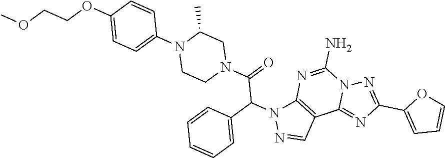 [SMILES: COCCOc1ccc(N2CCN(C(=O)C(c3ccccc3)n3ncc4c3nc(N)n3nc(-c5ccco5)nc43)C[C@H]2C)cc1]

To a mixture of (R)-1-(4-(2-methoxyethoxy)phenyl)-2-methylpiperazine (388 mg, 1.2 mmol), 2-(5-amino-2-(furan-2-yl)-7H-pyrazolo[4,3-e][1,2,4]triazolo[1,5-c]pyrimidin-7-yl)-2-phenylacetic acid (412 mg, 1.1 mmol), DIPEA (425 mg, 3.3 mmol) in DMF (5 mL) was added HATU (420 mg, 1.1 mmol) at rt and the mixture was stirred at rt for 16 hrs. The reaction was quenched with water (40 mL) to form a suspension. The suspension was filtered and the solid was purified by column chromatography (DCM:MeOH=30:1) to give the title product (490 mg, yield: 80.7%). ¹H NMR (400 MHz, DMSO-d6) δ 8.30-8.05 (m, 3H), 7.95 (s, 1H), 7.55-7.30 (m, 5H), 7.28-7.20 (m, 1H), 6.89 (s, 1H), 6.85-6.69 (m, 5H), 4.04-3.94 (m, 2H), 3.93-3.80 (m, 1H), 3.75-3.64 (m, 1H), 3.63-3.56 (m, 2H), 3.53-3.45 (m, 1H), 3.28 (s, 3H), 3.24-3.12 (m, 1H), 3.00-2.65 (m, 2H), 2.42-2.29 (m, 1H), 0.91-0.70 (m, 2H), 0.55-0.41 (m, 1H). MS: M/e 608 (M+1)⁺.

Compound A47 was separated into two enantiomeric stereoisomers, Compound A47a (fast isomer), and Compound A47b (slow isomer) by prep-SFC. The chiral separation

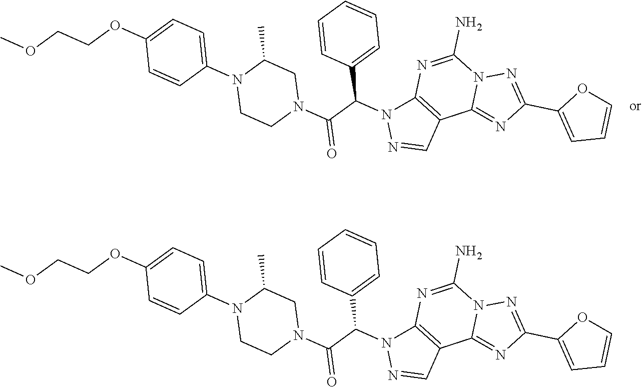 [SMILES: COCCOc1ccc(N2CCN(C(=O)[C@@H](c3ccccc3)n3ncc4c3nc(N)n3nc(-c5ccco5)nc43)C[C@H]2C)cc1 ; COCCOc1ccc(N2CCN(C(=O)[C@H](c3ccccc3)n3ncc4c3nc(N)n3nc(-c5ccco5)nc43)C[C@H]2C)cc1]

| Column | CHIRALPAK IA |
|---|---|
| Column size | 5 cm × 25 cm, 5 um |
| Injection | 10 mL |
| Mobile phase | CO₂:(MeOH:DCM = 1:1) = 50:50 |
| Flow rate | 180 g/min |
| Wavelength | UV 220 nm |
| Temperature | 25° C. |
| Sample solution | 8.1 mg/ml in MeOH:DCM = 3:1 |
| Prep-SFC equipment | Prep-SFC-350 |

### Compound A48: 2-(5-amino-2-(furan-2-yl)-7H-pyrazolo[4,3-e][1,2,4]triazolo[1,5-c]pyrimidin-7-yl)-1-(4-(2,4-difluorophenyl)piperazin-1-yl)-2-phenylethan-1-one

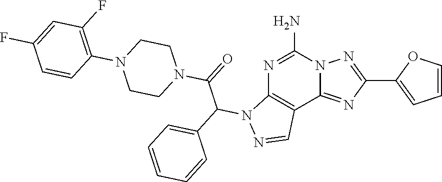 [SMILES: Nc1nc2c(cnn2C(C(=O)N2CCN(c3ccc(F)cc3F)CC2)c2ccccc2)c2nc(-c3ccco3)nn12]

A mixture of 2-(5-amino-2-(furan-2-yl)-7H-pyrazolo[4,3-e][1,2,4]triazolo[1,5-c]pyrimidin-7-yl)-2-phenylacetic acid (37.5 0.1 mmol), 1-(2,4-difluorophenyl)piperazine (19.8 mg, 0.1 mmol), HATU (46 mg, 0.12 mmol) and DIPEA (25.8 mg, 0.2 mmol) in DMF (3 mL) was stirred overnight. The reaction mixture was poured into H₂O (15 mL) and extracted with EtOAc (20 mL×3). The combined organic layers were washed with brine, dried over Na₂SO₄, concentrated and purified by column chromatography (petroleum ether/EtOAc=4:1˜100% EtOAc) to give the target compound (42 mg, 75.6%). ¹H NMR (400 MHz, DMSO-d6) δ 8.21 (s, 1H), 8.15 (s, 2H), 7.95 (d, J=0.8 Hz, 1H), 7.45-7.30 (m, 5H), 7.24 (d, J=3.2 Hz, 1H), 7.20-7.14 (m, 1H), 6.98-6.95 (m, 2H), 6.85 (s, 1H), 6.74 (m, 1H), 3.67 (m, 2H), 3.44-3.40 (m, 2H), 2.91 (m, 2H), 2.58 (m, 2H) ppm. MS: M/e 556 (M+1)⁺.

### Compound A49: 2-(5-amino-2-(furan-2-yl)-7H-pyrazolo[4,3-e][1,2,4]triazolo[1,5-c]pyrimidin-7-yl)-1-(4-(4-fluorophenyl)piperazin-1-yl)-2-phenylethan-1-one

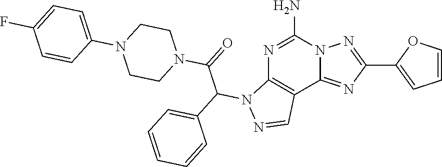 [SMILES: Nc1nc2c(cnn2C(C(=O)N2CCN(c3ccc(F)cc3)CC2)c2ccccc2)c2nc(-c3ccco3)nn12]

A mixture of 2-(5-amino-2-(furan-2-yl)-7H-pyrazolo[4,3-e][1,2,4]triazolo[1,5-c]pyrimidin-7-yl)-2-phenylacetic acid (50 mg, 0.133 mmol), 1-(4-fluorophenyl)piperazine hydrochloride (28.9 mg, 0.133 mmol), HATU (61 mg, 0.159 mmol) and DIPEA (34.3 mg, 0.266 mmol) in DMF (3 mL) was stirred overnight. The reaction mixture was poured into H₂O (10 mL) and extracted with EtOAc (15 mL×3). The combined organic layers were washed with brine, dried over Na₂SO₄, concentrated and purified by column chromatography (petroleum ether/EtOAc=1:1˜100% EtOAc) to give the target compound (20 mg, 28%). ¹H NMR (400 MHz, DMSO-d6) δ 8.20 (s, 1H), 8.16 (s, 1H), 7.95 (s, 1H), 7.40-7.30 (m, 5H), 7.24 (d, J=3.2 Hz, 1H), 7.02 (t, J=8.8 Hz, 2H), 6.89-6.82 (m, 3H), 6.74-6.71 (m, 1H), 3.71-3.55 (m, 2H), 3.49-3.32 (m, 2H), 3.01 (m, 2H), 2.72 (m, 2H) ppm. MS: M/e 538 (M+1)⁺.

### Compound A50: 2-(5-amino-2-(furan-2-yl)-7H-pyrazolo[4,3-e][1,2,4]triazolo[1,5-c]pyrimidin-7-yl)-1-((R)-4-(4-(2-methoxyethoxy)phenyl)-2-methylpiperazin-1-yl)-2-phenylethan-1-one

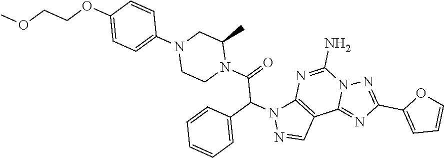 [SMILES: COCCOc1ccc(N2CCN(C(=O)C(c3ccccc3)n3ncc4c3nc(N)n3nc(-c5ccco5)nc43)[C@H](C)C2)cc1]

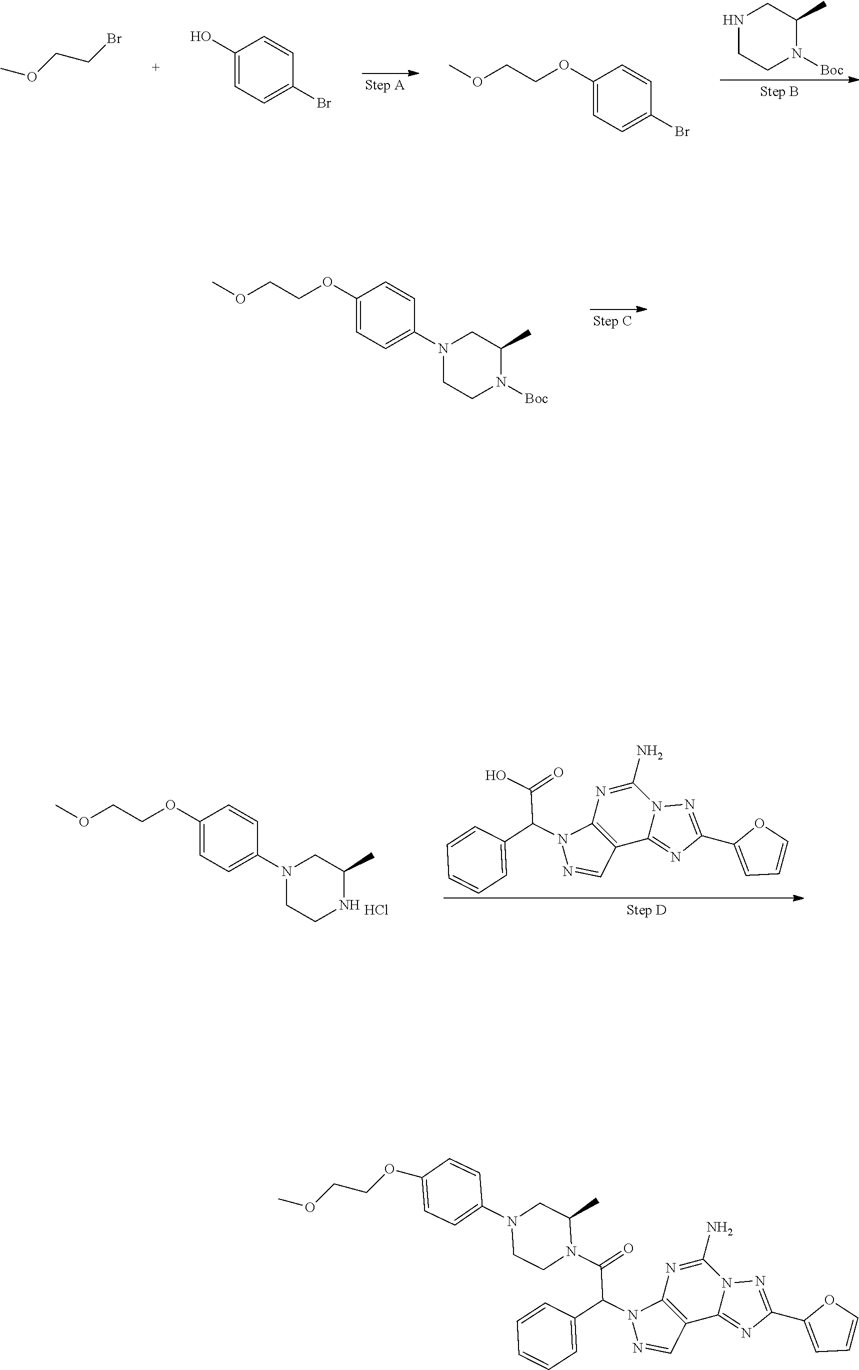

### Step A: 1-bromo-4-(2-methoxyethoxy)benzene

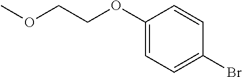

A mixture of 1-bromo-2-methoxyethane (5.0 g, 36.0 mmol), 4-bromophenol (5.0 g, 29.1 mmol), and K₂CO₃ (8.0 g, 58.0 mmol) in DMF (40 mL) was stirred at rt for 16 hrs. The mixture was diluted with 100 mL of PE, washed with NaOH (2M, 30 mL×2), brine (50 mL×3), dried over Na₂SO₄ and concentrated to give the title product (7.5 g, 95%) as a colorless oil. ¹H NMR (400 MHz, CDCl₃) δ 7.36 (d, J=8.8 Hz, 2H), 6.81 (d, J=8.8 Hz, 2H), 4.13-4.02 (m, 2H), 3.77-3.71 (m, 2H), 3.45 (s, 3H).

### Step B: tert-butyl (R)-4-(4-(2-methoxyethoxy)phenyl)-2-methylpiperazine-1-carboxylate

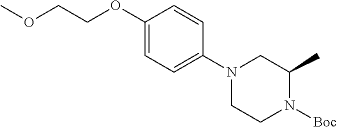 [SMILES: COCCOc1ccc(N2CCN(C(=O)OC(C)(C)C)[C@H](C)C2)cc1]

A mixture of 1-bromo-4-(2-methoxyethoxy)benzene (650 mg, 2.83 mmol), tert-butyl (R)-2-methylpiperazine-1-carboxylate (650 mg, 3.25 mmol), Pd₂(dba)₃ (250 mg, 0.27 mmol), X-phos (250 mg, 0.52 mmol) and Cs₂CO₃ (1.85 g, 5.7 mmol) in toluene (10 mL) was stirred at 90° C. under N₂ for 16 hrs. The mixture was concentrated, the resulting residue was diluted with EA (30 mL) and the resulting suspension was filtered. The filtrate was concentrated and purified by column chromatography to give the title product (580 mg, crude) as a yellow oil. MS: M/e 351 (M+1)⁺.

### Step C: (R)-1-(4-(2-methoxyethoxy)phenyl)-3-methylpiperazine hydrochloride

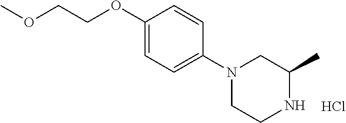

To a solution of tert-butyl (R)-4-(4-(2-methoxyethoxy)phenyl)-2-methylpiperazine-1-carboxylate (570 mg, 1.62 mmol) in CH₂Cl₂ (5 mL) was added CF₃COOH (3 mL) at rt and the resulting red solution was stirred at rt for 3 hrs. The mixture was concentrated. 10 mL of HCl (2M, aq.) was added and the solution was extracted with EA (10 mL×2). The organic layer was removed and the aqueous layer was concentrated to dryness to give the title product (280 mg, 62%) as a light brown oil. MS: M/e 251 (M+1)⁺.

### Step D: 2-(5-amino-2-(furan-2-yl)-7H-pyrazolo[4,3-e][1,2,4]triazolo[1,5-c]pyrimidin-7-yl)-1-((R)-4-(4-(2-methoxyethoxy)phenyl)-2-methylpiperazin-1-yl)-2-phenylethan-1-one

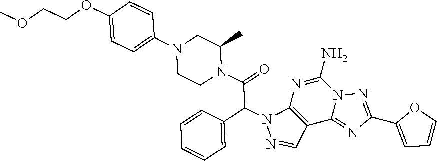 [SMILES: COCCOc1ccc(N2CCN(C(=O)C(c3ccccc3)n3ncc4c3nc(N)n3nc(-c5ccco5)nc43)[C@H](C)C2)cc1]

To a mixture of (R)-1-(4-(2-methoxyethoxy)phenyl)-3-methylpiperazine hydrochloride (280 mg, 1.0 mmol), 2-(5-amino-2-(furan-2-yl)-7H-pyrazolo[4,3-e][1,2,4]triazolo[1,5-c]pyrimidin-7-yl)-2-phenylacetic acid (400 mg, 1.1 mmol), DIEA (500 mg, 3.9 mmol) in DMF (5 mL) was added HATU (460 mg, 1.2 mmol) at rt and the mixture was stirred at rt for 16 hrs. The mixture was poured into 30 mL of H₂O. A white solid precipitated and which was filtered. The filter cake was washed with H₂O (20 mL), dried in air. The resulting solid was purified by column chromatography to give the title product (315 mg, yield: 52%). ¹H NMR (400 MHz, DMSO-d6) δ 8.29-8.01 (m, 3H), 7.95 (s, 1H), 7.49-7.29 (m, 5H), 7.24 (s, 1H), 6.90-6.77 (m, 5H), 6.74 (s, 1H), 4.77-4.30 (m, 1H), 4.00-3.94 (m, 2H), 3.62-3.57 (m, 2H), 3.55-3.41 (m, 1H), 3.28 (s, 3H), 3.24-2.61 (m, 3H), 2.47-2.15 (m, 2H), 1.22 (d, J=7.2 Hz, 2H), 1.12 (d, J=6.4 Hz, 1H). MS: M/e 608 (M+1)⁺.

Compound A50 was separated into two enantiomeric stereoisomers, Compound A50a (earlier peak), and Compound A50b (later peak) by chiral prep-HPLC. The chiral separation conditions are shown below.

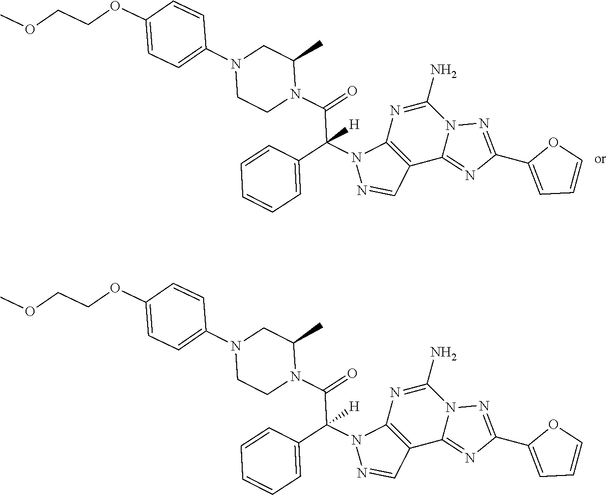 [SMILES: COCCOc1ccc(N2CCN(C(=O)[C@@H](c3ccccc3)n3ncc4c3nc(N)n3nc(-c5ccco5)nc43)[C@H](C)C2)cc1 ; COCCOc1ccc(N2CCN(C(=O)[C@H](c3ccccc3)n3ncc4c3nc(N)n3nc(-c5ccco5)nc43)[C@H](C)C2)cc1]

| Column | CHIRALPAK IC |
|---|---|
| Column size | 2 cm × 25 cm, 5 um |
| Injection | 0.6 mL |
| Mobile phase | (Hex:DCM = 3:1):EtOH = 50:50 |
| Flow rate | 18 ml/min |
| Wavelength | UV 220 nm |
| Temperature | 25° C. |
| Sample solution | 66.7 mg/ml in EtOH:DCM = 3:1 |
| Prep-HPLC equipment | BJ-Prep-Gilson-HPLC |

### Compound A51: 2-(5-amino-2-(furan-2-yl)-7H-pyrazolo[4,3-e][1,2,4]triazolo[1,5-c]pyrimidin-7-yl)-1-(4-(2,3-difluoro-4-(2-methoxyethoxy)phenyl)piperazin-1-yl)pentan-1-one

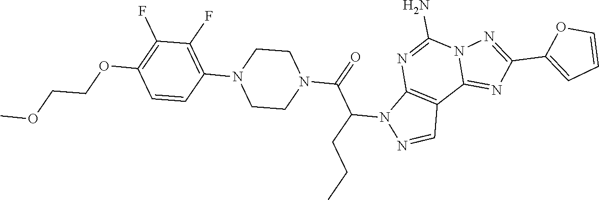 [SMILES: CCCC(C(=O)N1CCN(c2ccc(OCCOC)c(F)c2F)CC1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)pentanoic acid (50 mg, 0.15 mmol), 1-(2,3-difluoro-4-(2-methoxyethoxy)phenyl)piperazine hydrochloride (50 mg, 0.16 mmol), HATU (61 mg, 0.16 mmol) and DIEA (39 mg, 0.3 mmol) in DMF (5 mL) was stirred at rt for 2 hrs. The solution was added with water (10 mL), extracted with ethyl acetate (10 mL) and washed with brine (10 mL). The organic layer was dried, concentrated and purified by column chromatography (PE:EA=1:1) to get the desired product (31 mg, 36%). ¹H NMR (400 MHz, DMSO-d6) δ 8.21 (s, 1H), 8.15 (br.s, 2H), 7.95 (s, 1H), 7.23 (d, J=4.0 Hz, 1H), 6.87-6.66 (m, 3H), 5.58 (dd, J=8.0 Hz, 4.0 Hz, 1H), 4.12-4.09 (m, 2H), 3.62-3.50 (m, 6H), 3.28 (s, 3H), 2.89-2.67 (m, 3H), 2.36 (br.s, 1H), 2.03-1.99 (m, 2H), 1.23-1.10 (m, 2H), 0.87 (t, J=4.0 Hz, 3H) ppm. MS: M/e 596 (M+1)⁺

### Compound A52: 2-(5-amino-2-(furan-2-yl)-7H-pyrazolo[4,3-e][1,2,4]triazolo[1,5-c]pyrimidin-7-yl)-1-((S)-4-(4-(2-methoxyethoxy)phenyl)-2-methylpiperazin-1-yl)-2-phenylethan-1-one

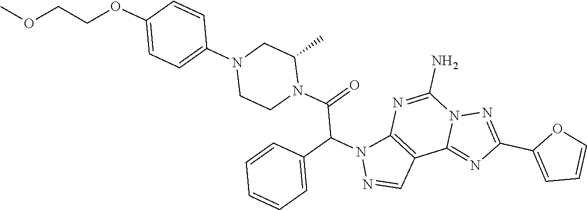 [SMILES: COCCOc1ccc(N2CCN(C(=O)C(c3ccccc3)n3ncc4c3nc(N)n3nc(-c5ccco5)nc43)[C@@H](C)C2)cc1]

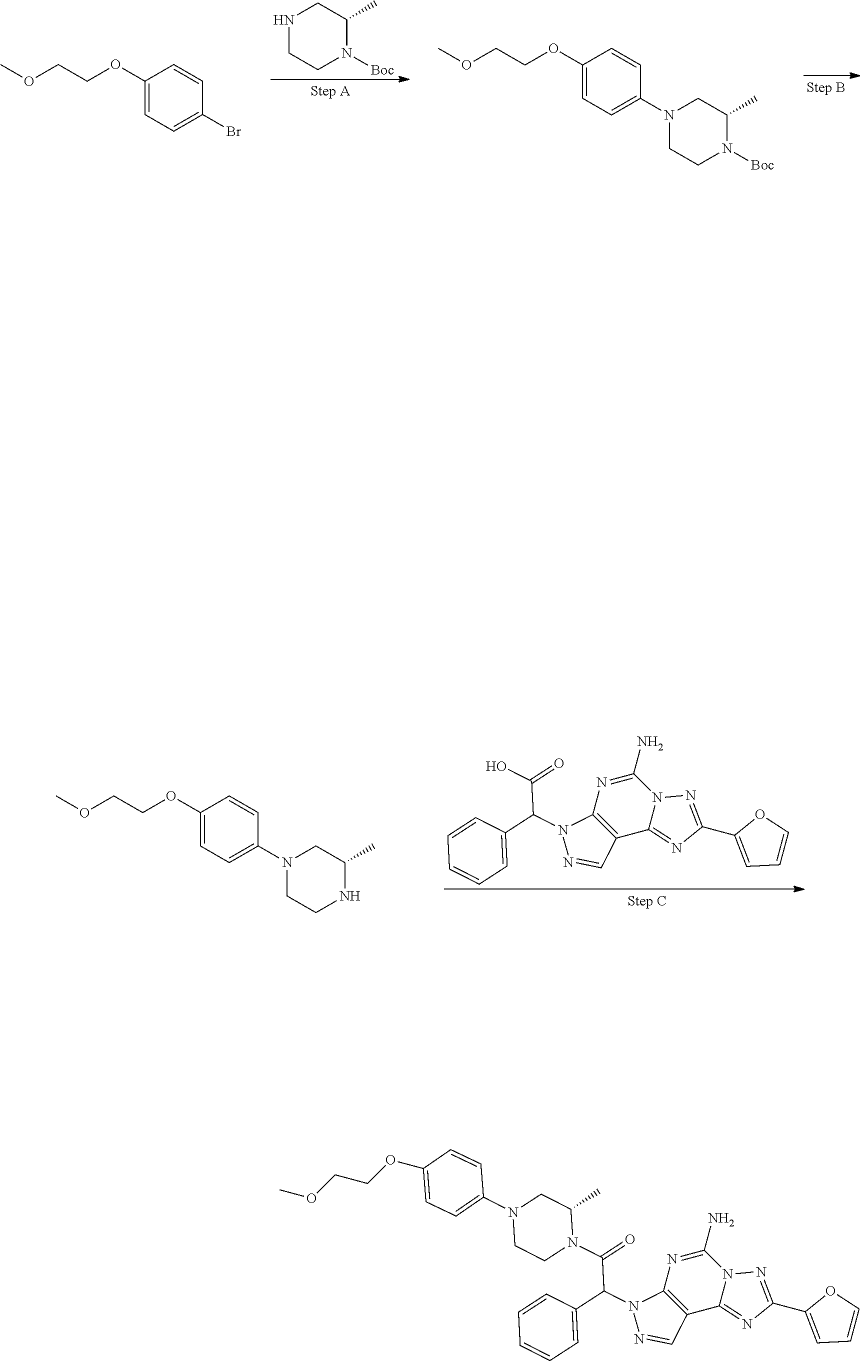

### Step A: tert-butyl (S)-4-(4-(2-methoxyethoxy)phenyl)-2-methylpiperazine-1-carboxylate

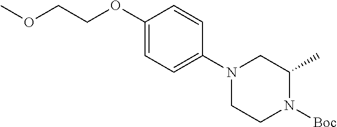 [SMILES: COCCOc1ccc(N2CCN(C(=O)OC(C)(C)C)[C@@H](C)C2)cc1]

A mixture of 1-bromo-4-(2-methoxyethoxy)benzene (230 mg, 1.0 mmol), tert-butyl (S)-2-methylpiperazine-1-carboxylate (230 mg, 1.1 mmol), Pd₂(dba)₃ (88 mg, 0.09 mmol), X-phos (90 mg, 0.20 mmol) and Cs₂CO₃ (650 mg, 2 mmol) in toluene (4 mL) was stirred at 90° C. under N₂ for 16 hrs. The mixture was diluted with EA (20 mL) and the resulting suspension was filtered. The filtrate was concentrated and purified by column chromatography to give the title product (120 mg, crude) as a light yellow oil. MS: M/e 351 (M+1)⁺.

### Step B: (S)-1-(4-(2-methoxyethoxy)phenyl)-3-methylpiperazine

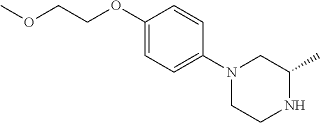

To a solution of tert-butyl (S)-4-(4-(2-methoxyethoxy)phenyl)-2-methylpiperazine-1-carboxylate (120 mg, 0.34 mmol) in CH₂Cl₂ (5 mL) was added CF₃COOH (2 mL) at rt and the resulting solution was stirred at rt for 16 hrs. The mixture was concentrated to dryness to give the title product (95 mg, crude) as a light yellow oil which was used for the next step directly. MS: M/e 251 (M+1)⁺.

### Step C: 2-(5-amino-2-(furan-2-yl)-7H-pyrazolo[4,3-e][1,2,4]triazolo[1,5-c]pyrimidin-7-yl)-1-((S)-4-(4-(2-methoxyethoxy)phenyl)-2-methylpiperazin-1-yl)-2-phenylethan-1-one

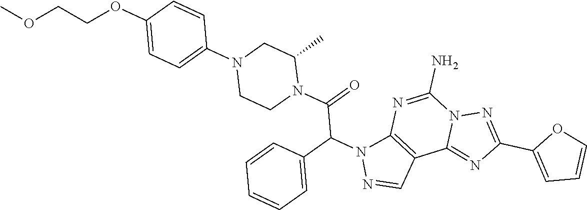 [SMILES: COCCOc1ccc(N2CCN(C(=O)C(c3ccccc3)n3ncc4c3nc(N)n3nc(-c5ccco5)nc43)[C@@H](C)C2)cc1]

To a mixture of (S)-1-(4-(2-methoxyethoxy)phenyl)-3-methylpiperazine (95 mg, 0.26 mmol), 2-(5-amino-2-(furan-2-yl)-7H-pyrazolo[4,3-e][1,2,4]triazolo[1,5-c]pyrimidin-7-yl)-2-phenylacetic acid (105 mg, 0.28 mmol), DIEA (150 mg, 1.2 mmol) in DMF (2 mL) was added HATU (120 mg, 0.32 mmol) at rt and the mixture was stirred at rt for 16 hrs. The mixture was diluted with 10 mL of EA and washed with NaHCO₃ (aq. 5 mL×2), brine (5 mL×3), dried over Na₂SO₄ and concentrated, purified by prep-TLC to give the title product (25 mg, yield: 15%). ¹H NMR (400 MHz, DMSO-d6) δ 8.31-8.03 (m, 3H), 7.95 (s, 1H), 7.51-7.28 (m, 5H), 7.24 (s, 1H), 6.91-6.69 (m, 6H), 4.74-4.29 (m, 1H), 4.26-3.83 (m, 3H), 3.70-3.49 (m, 3H), 3.28 (s, 3H), 3.15-2.85 (m, 2H), 2.71-2.59 (m, 1H), 2.35-2.14 (m, 1H), 1.27-1.09 (m, 3H). MS: M/e 608 (M+1)⁺.

### Compound A53: 2-(5-amino-2-(furan-2-yl)-7H-pyrazolo[4,3-e][1,2,4]triazolo[1,5-c]pyrimidin-7-yl)-1-(4-(3-fluoro-4-(2-methoxyethoxy)phenyl)piperazin-1-yl)pentan-1-one

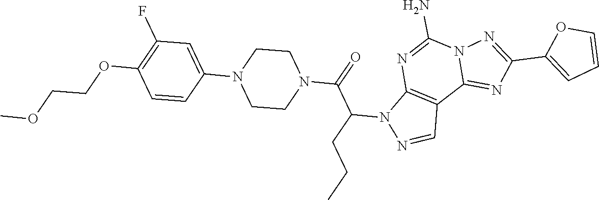 [SMILES: CCCC(C(=O)N1CCN(c2ccc(OCCOC)c(F)c2)CC1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)pentanoic acid (50 mg, 0.15 mmol), 1-(3-fluoro-4-(2-methoxyethoxy)phenyl)piperazine hydrochloride (46 mg, 0.16 mmol), HATU (61 mg, 0.16 mmol) and DIEA (39 mg, 0.32 mmol) in DMF (5 mL) was stirred at rt for 2 hrs. The solution was added with water (5 mL), extracted with ethyl acetate (10 mL) and washed with brine (10 mL). The organic layer was dried, concentrated and purified by column chromatography (PE:EA=1:1) to get the desired product (43 mg, 51%). ¹H NMR (400 MHz, DMSO-d6) δ 8.20 (s, 1H), 8.18 (br.s, 2H), 7.95 (s, 1H), 7.23 (d, J=4.0 Hz, 1H), 6.97-6.95 (m, 1H), 6.73-6.57 (m, 3H), 5.58 (dd, J=8.0 Hz, 4.0 Hz, 1H), 4.09-4.02 (m, 2H), 3.61-3.51 (m, 6H), 3.28 (s, 3H), 3.01-2.83 (m, 3H), 2.44 (br.s, 1H), 2.16-2.00 (m, 2H), 1.23-1.09 (m, 2H), 0.91-0.85 (m, 3H) ppm. MS: M/e 578 (M+1)⁺

### Compound A54: 2-(5-amino-2-(furan-2-yl)-7H-pyrazolo[4,3-e][1,2,4]triazolo[1,5-c]pyrimidin-7-yl)-1-(4-(2,5-difluoro-4-(2-methoxyethoxy)phenyl)piperazin-1-yl)pentan-1-one

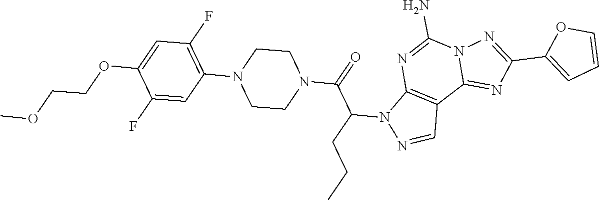 [SMILES: CCCC(C(=O)N1CCN(c2cc(F)c(OCCOC)cc2F)CC1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)pentanoic acid (50 mg, 0.15 mmol), 1-(2,5-difluoro-4-(2-methoxyethoxy)phenyl)piperazine hydrochloride (54 mg, 0.18 mmol), HATU (69 mg, 0.18 mmol) and DIEA (39 mg, 0.3 mmol) in DMF (10 mL) was stirred at rt for 2 hrs. The solution was added with water (10 mL), extracted with ethyl acetate (10 mL) and washed with brine (10 mL). The organic layer was dried, concentrated and purified by column chromatography (PE:EA=1:1) to get the desired product (49 mg, 56%). ¹H NMR (400 MHz, DMSO-d6) δ 8.21 (s, 1H), 8.15 (br.s, 2H), 7.95 (s, 1H), 7.23 (d, J=4.0 Hz, 1H), 7.13-7.07 (m, 1H), 6.94-6.88 (m, 1H), 6.74-6.73 (m, 1H), 5.58 (dd, J=8.0 Hz, 4.0 Hz, 1H), 4.09 (t, J=4.0 Hz, 2H), 3.61-3.51 (m, 6H), 3.28 (s, 3H), 2.89-2.73 (m, 2H), 2.41 (br.s, 1H), 2.21-1.99 (m, 2H), 1.23-1.09 (m, 2H), 0.90-0.86 (m, 3H) ppm. MS: M/e 596 (M+1)⁺

### Compound A55: 2-(5-amino-2-(furan-2-yl)-7H-pyrazolo[4,3-e][1,2,4]triazolo[1,5-c]pyrimidin-7-yl)-1-(4-(3-fluoro-4-(2-methoxyethoxy)phenyl)piperazin-1-yl)-2-phenylethan-1-one

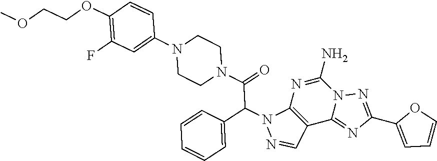 [SMILES: COCCOc1ccc(N2CCN(C(=O)C(c3ccccc3)n3ncc4c3nc(N)n3nc(-c5ccco5)nc43)CC2)cc1F]

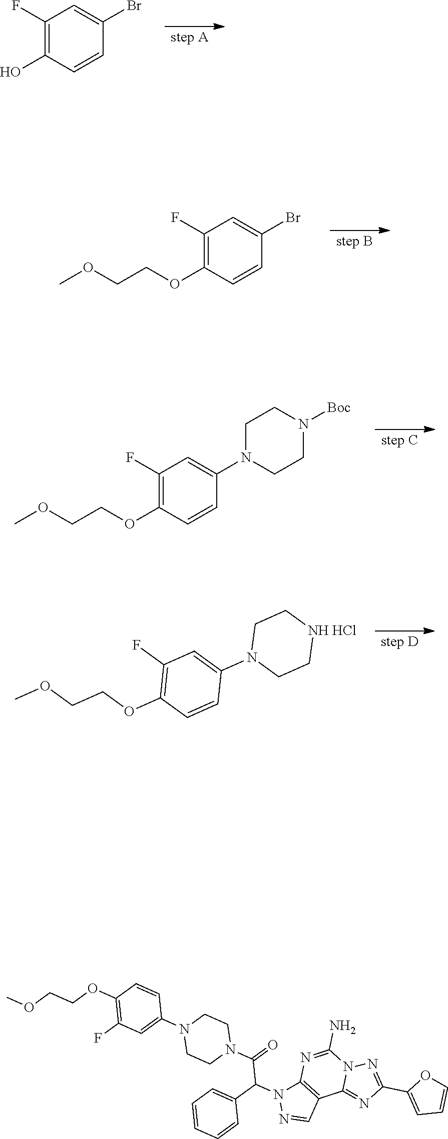

### Step A: 4-bromo-2-fluoro-1-(2-methoxyethoxy)benzene

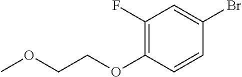

A mixture of 4-bromo-2-fluorophenol (2 g, 10.47 mmol), 1-bromo-2-methoxyethane (1.6 g, 11.52 mmol) and K₂CO₃ (2.89 g, 20.94 mmol) in DMF (10 mL) was stirred at 60° C. overnight. The reaction mixture was poured into H₂O (20 mL) and extracted with EtOAc (20 mL×3). The combined organic layers were washed with brine, dried over Na₂SO₄, concentrated under high vacuum to give the target compound (2.6 g, 99%) as colorless oil.

### Step B: tert-butyl 4-(3-fluoro-4-(2-methoxyethoxy)phenyl)piperazine-1-carboxylate

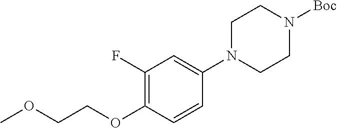 [SMILES: COCCOc1ccc(N2CCN(C(=O)OC(C)(C)C)CC2)cc1F]

A mixture of the product of step A (2.6 g, 10.47 mmol), tert-butyl piperazine-1-carboxylate (2.6 g, 10.47 mmol), Pd₂(dba)₃ (0.95 g, 1.04 mmol), X-phos (0.99 g, 2.08 mmol) and Cs₂CO₃ (6.8 g, 20.94 mmol) in toluene (150 mL) was stirred at 120° C. for 2 hours. Most toluene was removed to give the residue, which was treated with EtOAc/H₂O (60 mL/30 mL). The organic layer was separated, washed with brine, dried over Na₂SO₄, concentrated and purified by column chromatography (petroleum ether/EtOAc=5:1˜3:1) to give the target compound (2 g, 54%) as yellow oil. MS: M/e 355 (M+1)⁺.

### Step C: 1-(3-fluoro-4-(2-methoxyethoxy)phenyl)piperazine Hydrochloride

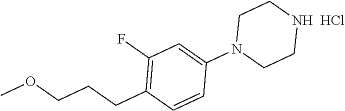 [SMILES: COCCCc1ccc(N2CCNCC2)cc1F ; Cl]

To a stirred mixture of the product of step B (2 g, 5.65 mmol) in CH₂Cl₂ (10 mL) was added EtOAc/HCl (g) (4.0 M, 10 mL). After the addition, the reaction mixture was stirred overnight. The reaction mixture was filtered and the cake was collected, dried to give the target compound (1.47 g, 90%) as a tan solid. MS: M/e 255 (M+1)⁺.

### Step D: 2-(5-amino-2-(furan-2-yl)-7H-pyrazolo[4,3-e][1,2,4]triazolo[1,5-c]pyrimidin-7-yl)-1-(4-(3-fluoro-4-(2-methoxyethoxy)phenyl)piperazin-1-yl)-2-phenylethan-1-one

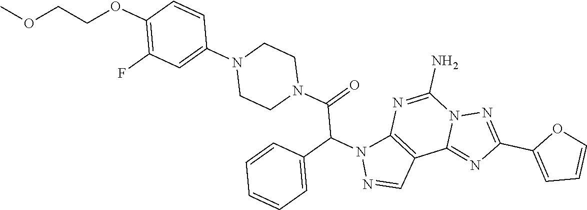 [SMILES: COCCOc1ccc(N2CCN(C(=O)C(c3ccccc3)n3ncc4c3nc(N)n3nc(-c5ccco5)nc43)CC2)cc1F]

A mixture of 2-(5-amino-2-(furan-2-yl)-7H-pyrazolo[4,3-e][1,2,4]triazolo[1,5-c]pyrimidin-7-yl)-2-phenylacetic acid (37.5 mg, 0.1 mmol), the product of step C (29 mg, 0.1 mmol), HATU (46 mg, 0.12 mmol) and DIPEA (25.8 mg, 0.2 mmol) in DMF (3 mL) was stirred overnight. The reaction mixture was poured into H₂O (10 mL) and extracted with EtOAc (15 mL×3). The combined organic layers were washed with brine, dried over Na₂SO₄, concentrated and purified by column chromatography (petroleum ether/EtOAc=2:1˜100% EtOAc) to give the target compound (25 mg, 40.9%). ¹H NMR (400 MHz, DMSO-d6) δ 8.21 (s, 1H), 8.16 (s, 2H), 7.95 (s, 1H), 7.43-7.31 (m, 5H), 7.24 (d, J=3.2 Hz, 1H), 7.00-6.96 (t, J=9.2 Hz, 1H), 6.86 (s, 1H), 6.81-6.73 (m, 2H), 6.58-6.56 (m, 1H), 4.07-4.00 (m, 2H), 3.65-3.59 (m, 4H), 3.41 (m, 2H), 3.28 (s, 3H), 3.01 (m, 2H), 2.68 (m, 2H) ppm. MS: M/e 612 (M+1)⁺.

### Compound A56: 2-(5-amino-2-(furan-2-yl)-7H-pyrazolo[4,3-e][1,2,4]triazolo[1,5-c]pyrimidin-7-yl)-1-(4-(2,3-difluoro-4-(2-methoxyethoxy)phenyl)piperazin-1-yl)-2-phenylethan-1-one

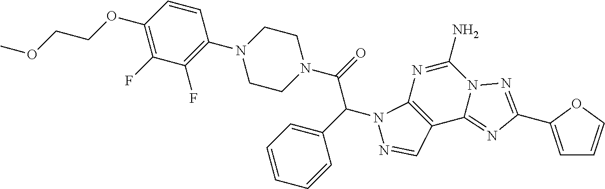 [SMILES: COCCOc1ccc(N2CCN(C(=O)C(c3ccccc3)n3ncc4c3nc(N)n3nc(-c5ccco5)nc43)CC2)c(F)c1F]

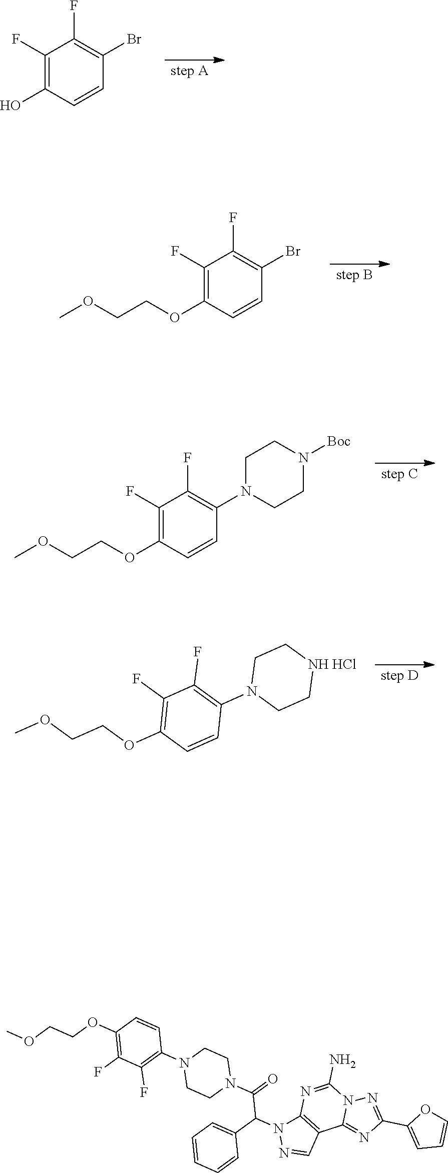

### Step A: 1-bromo-2,3-difluoro-4-(2-methoxyethoxy)benzene

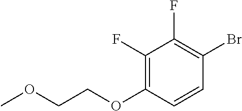

A mixture of 4-bromo-2,3-difluorophenol (2 g, 9.56 mmol), 1-bromo-2-methoxyethane (1.46 g, 10.52 mmol) and K₂CO₃ (2.6 g, 19.12 mmol) in DMF (10 mL) was stirred at 60° C. overnight. The reaction mixture was poured into H₂O (30 mL) and extracted with EtOAc (20 mL×3). The combined organic layers were washed with brine, dried over Na₂SO₄, concentrated under high vacuum to give the target compound (2.55 g, 100%) as colorless oil.

### Step B: tert-butyl 4-(2,3-difluoro-4-(2-methoxyethoxy)phenyl)piperazine-1-carboxylate

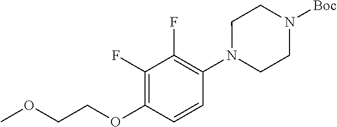 [SMILES: COCCOc1ccc(N2CCN(C(=O)OC(C)(C)C)CC2)c(F)c1F]

A mixture of the product of step A (1 g, 3.73 mmol), tert-butyl piperazine-1-carboxylate (0.69 g, 3.73 mmol), Pd₂(dba)₃ (0.34 g, 0.373 mmol), X-phos (0.35 g, 0.746 mmol) and Cs₂CO₃ (2.4 g, 7.46 mmol) in toluene (50 mL) was stirred at 120° C. for 2 hours under N₂. Most toluene was removed to give the residue, which was treated with EtOAc/H₂O (60 mL/30 mL). The organic layer was separated, washed with brine, dried over Na₂SO₄, concentrated and purified by column chromatography (petroleum ether/EtOAc=5:1˜3:1) to give the target compound (620 mg, 44.7%) as yellow oil. MS: M/e 373 (M+1)⁺.

### Step C: 1-(2,3-difluoro-4-(2-methoxyethoxy)phenyl)piperazine Hydrochloride

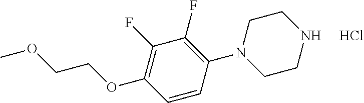 [SMILES: COCCOc1ccc(N2CCNCC2)c(F)c1F ; Cl]

To a stirred solution of the product of step B (620 mg, 1.67 mmol) in CH₂Cl₂ (10 mL) was added EtOAc/HCl (g) (4.0 M, 5 mL). After the addition, the reaction mixture was stirred overnight. The reaction mixture was filtered and the cake was collected, dried to give the target compound (323 mg, 62.7%) as a white solid. MS: M/e 273 (M+1)⁺.

### Step D: 2-(5-amino-2-(furan-2-yl)-7H-pyrazolo[4,3-e][1,2,4]triazolo[1,5-c]pyrimidin-7-yl)-1-(4-(2,3-difluoro-4-(2-methoxyethoxy)phenyl)piperazin-1-yl)-2-phenylethan-1-one

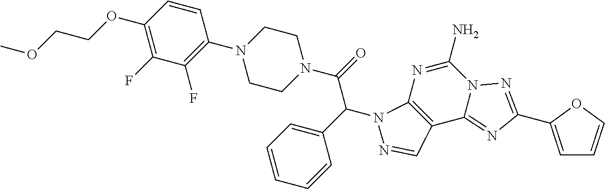 [SMILES: COCCOc1ccc(N2CCN(C(=O)C(c3ccccc3)n3ncc4c3nc(N)n3nc(-c5ccco5)nc43)CC2)c(F)c1F]

A mixture of 2-(5-amino-2-(furan-2-yl)-7H-pyrazolo[4,3-e][1,2,4]triazolo[1,5-c]pyrimidin-7-yl)-2-phenylacetic acid (37.5 mg, 0.1 mmol), the product of step C (30.8 mg, 0.1 mmol), HATU (46 mg, 0.12 mmol) and DIPEA (25.8 mg, 0.2 mmol) in DMF (3 mL) was stirred overnight. The reaction mixture was poured into H₂O (10 mL) and extracted with EtOAc (30 mL×2). The combined organic layers were washed with brine, dried over Na₂SO₄, concentrated and purified by column chromatography (petroleum ether/EtOAc=2:1˜1:2) to give the target compound (20 mg, 31.7%). ¹H NMR (400 MHz, DMSO-d6) δ 8.21 (s, 1H), 8.15 (s, 2H), 7.95 (s, 1H), 7.43-7.33 (m, 5H), 7.24 (d, J=3.2 Hz, 1H), 6.92-6.85 (m, 2H), 6.74 (dd, J=3.2, 1.6 Hz, 1H), 6.68 (m, 1H), 4.16-4.09 (m, 2H), 3.74-3.59 (m, 4H), 3.45-3.40 (m, 2H), 3.29 (s, 3H), 2.90 (m, 2H), 2.55 (m, 2H) ppm. MS: M/e 630 (M+1)⁺.

### Compound A57: 2-(5-amino-2-(furan-2-yl)-7H-pyrazolo[4,3-e][1,2,4]triazolo[1,5-c]pyrimidin-7-yl)-1-(4-(2,5-difluoro-4-(2-methoxyethoxy)phenyl)piperazin-1-yl)-2-phenylethan-1-one

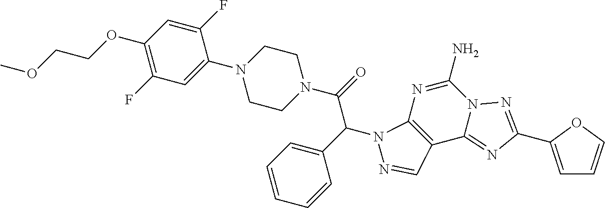 [SMILES: COCCOc1cc(F)c(N2CCN(C(=O)C(c3ccccc3)n3ncc4c3nc(N)n3nc(-c5ccco5)nc43)CC2)cc1F]

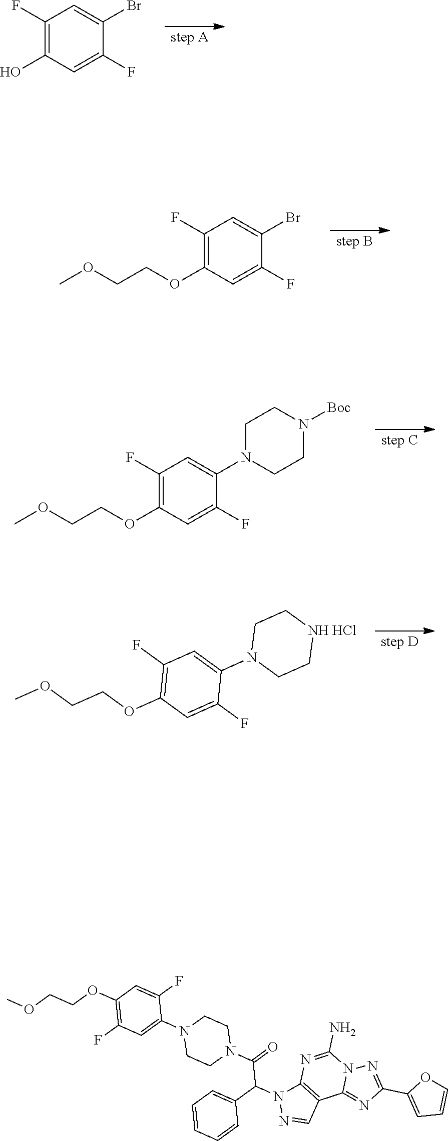

### Step A: 1-bromo-2,5-difluoro-4-(2-methoxyethoxy)benzene

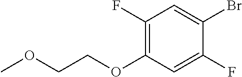

A mixture of 4-bromo-2,5-difluorophenol (2 g, 9.56 mmol), 1-bromo-2-methoxyethane (1.46 g, 10.52 mmol) and K₂CO₃ (2.6 g, 19.12 mmol) in DMF (10 mL) was stirred at 60° C. overnight. The reaction mixture was poured into H₂O (20 mL) and extracted with EtOAc (20 mL×3). The combined organic layers were washed with brine, dried over Na₂SO₄, concentrated under high vacuum to give the target compound (2.59 g, 99%) as colorless oil.

### Step B: tert-butyl 4-(2,5-difluoro-4-(2-methoxyethoxy)phenyl)piperazine-1-carboxylate

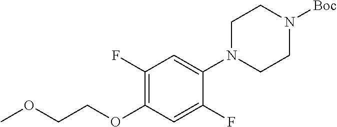 [SMILES: COCCOc1cc(F)c(N2CCN(C(=O)OC(C)(C)C)CC2)cc1F]

A mixture of the product of step A (1 g, 3.73 mmol), tert-butyl piperazine-1-carboxylate (0.69 g, 3.73 mmol), Pd₂(dba)₃ (0.34 g, 0.373 mmol), X-phos (0.35 g, 0.746 mmol) and Cs₂CO₃ (2.4 g, 7.46 mmol) in toluene (50 mL) was stirred at 120° C. for 2 hours under N₂. Most toluene was removed to give the residue, which was treated with EtOAc/H₂O (50 mL/20 mL). The organic layer was separated, washed with brine, dried over Na₂SO₄, concentrated and purified by column chromatography (petroleum ether/EtOAc=5:1˜3:1) to give the target compound (583 mg, 42%) as yellow oil. MS: M/e 373 (M+1)⁺.

### Step C: 1-(2,5-difluoro-4-(2-methoxyethoxy)phenyl)piperazine Hydrochloride

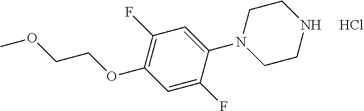 [SMILES: COCCOc1cc(F)c(N2CCNCC2)cc1F ; Cl]

To a stirred mixture of the product of step B (583 mg, 1.56 mmol) in CH₂Cl₂ (10 mL) was added EtOAc/HCl (g) (4.0 M, 5 mL). After the addition, the reaction mixture was stirred overnight. The reaction mixture was filtered and the cake was collected, dried to give the target compound (300 mg, 62.3%) as a white solid. MS: M/e 273 (M+1)⁺.

### Step D: 2-(5-amino-2-(furan-2-yl)-7H-pyrazolo[4,3-e][1,2,4]triazolo[1,5-c]pyrimidin-7-yl)-1-(4-(2,5-difluoro-4-(2-methoxyethoxy)phenyl)piperazin-1-yl)-2-phenylethan-1-one

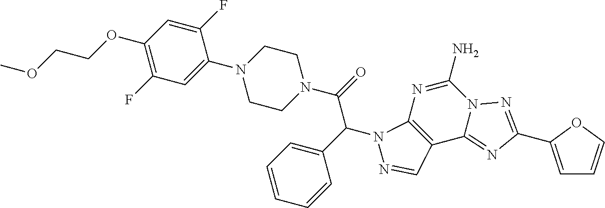 [SMILES: COCCOc1cc(F)c(N2CCN(C(=O)C(c3ccccc3)n3ncc4c3nc(N)n3nc(-c5ccco5)nc43)CC2)cc1F]

A mixture of 2-(5-amino-2-(furan-2-yl)-7H-pyrazolo[4,3-e][1,2,4]triazolo[1,5-c]pyrimidin-7-yl)-2-phenylacetic acid (37.5 mg, 0.1 mmol), the product of step C (30.8 mg, 0.1 mmol), HATU (46 mg, 0.12 mmol) and DIPEA (25.8 mg, 0.2 mmol) in DMF (3 mL) was stirred overnight. The reaction mixture was poured into H₂O (10 mL) and extracted with EtOAc (15 mL×3). The combined organic layers were washed with brine, dried over Na₂SO₄, concentrated and purified by column chromatography (petroleum ether/EtOAc=2:1˜1:2) to give the target compound (25 mg, 40.9%). ¹H NMR (400 MHz, DMSO-d6) δ 8.21 (s, 1H), 8.15 (s, 2H), 7.95 (s, 1H), 7.41-7.32 (m, 5H), 7.24-7.23 (d, J=3.2 Hz, 1H), 7.13-7.07 (m, 1H), 6.91-6.84 (m, 2H), 6.73 (m, 1H), 4.09 (m, 2H), 3.73-3.56 (m, 4H), 3.39 (m, 2H), 3.29 (s, 3H), 2.89 (m, 2H), 2.57 (m, 2H) ppm. MS: M/e 630 (M+1)⁺.

### Compound A58: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(piperidin-4-yl)ethan-1-one

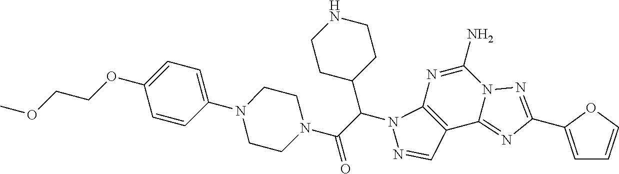 [SMILES: COCCOc1ccc(N2CCN(C(=O)C(C3CCNCC3)n3ncc4c3nc(N)n3nc(-c5ccco5)nc43)CC2)cc1]

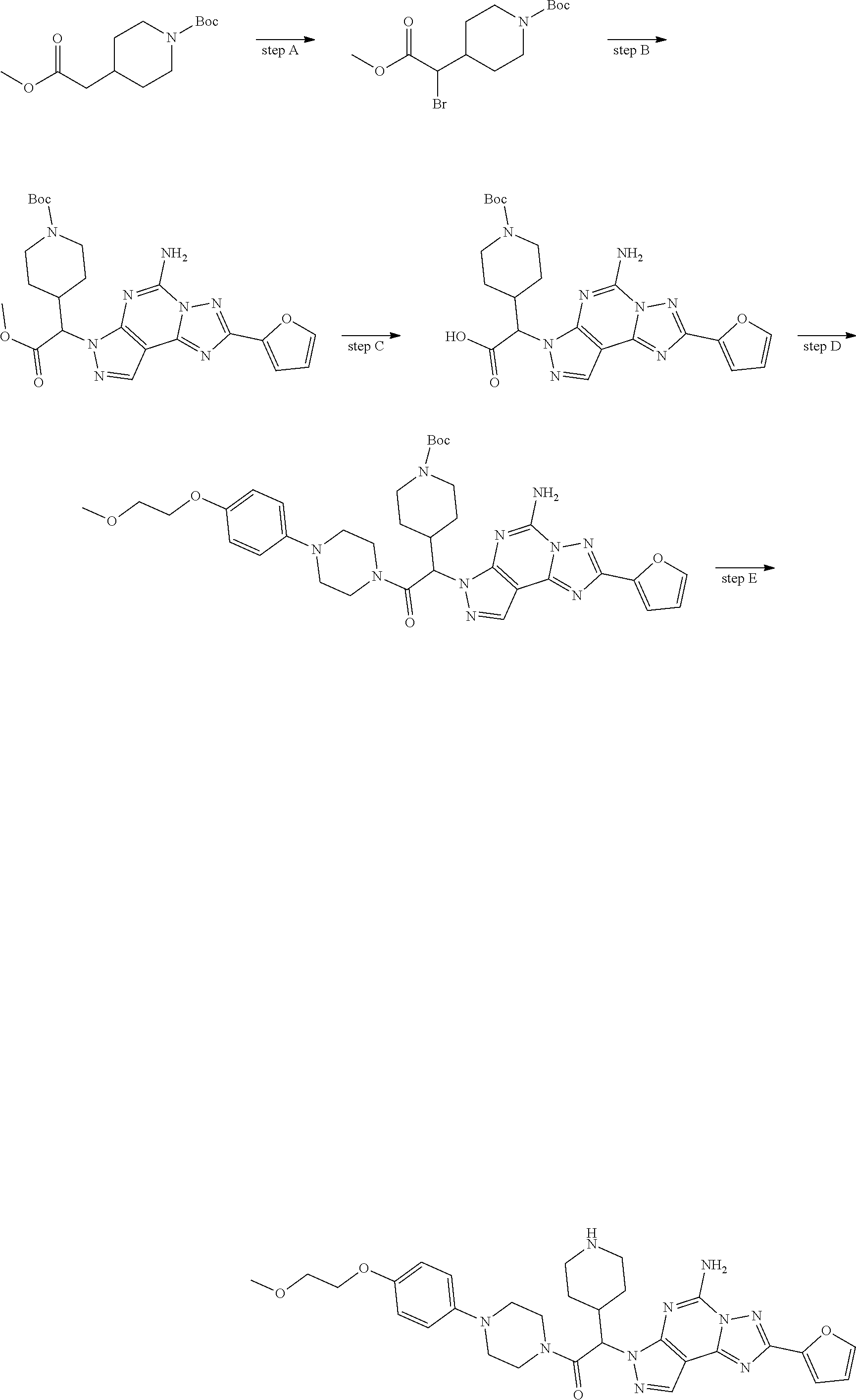 [SMILES: COC(=O)CC1CCN(C(=O)OC(C)(C)C)CC1 ; COC(=O)C(Br)C1CCN(C(=O)OC(C)(C)C)CC1 ; COC(=O)C(C1CCN(C(=O)OC(C)(C)C)CC1)n1ncc2c1nc(N)n1nc(-c3ccco3)nc21 ; CC(C)(C)OC(=O)N1CCC(C(C(=O)O)n2ncc3c2nc(N)n2nc(-c4ccco4)nc32)CC1 ; COCCOc1ccc(N2CCN(C(=O)C(C3CCN(C(=O)OC(C)(C)C)CC3)n3ncc4c3nc(N)n3nc(-c5ccco5)nc43)CC2)cc1 ; COCCOc1ccc(N2CCN(C(=O)C(C3CCNCC3)n3ncc4c3nc(N)n3nc(-c5ccco5)nc43)CC2)cc1]

### Step A: tert-butyl 4-(1-bromo-2-methoxy-2-oxoethyl)piperidine-1-carboxylate

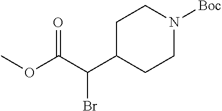 [SMILES: COC(=O)C(Br)C1CCN(C(=O)OC(C)(C)C)CC1]

A solution of tert-butyl 4-(2-methoxy-2-oxoethyl)piperidine-1-carboxylate (2.57 g, 10 mmol) in THF (10 mL) was added dropwise to LHMDS (17.5 mL, 1.0 M) at −78° C., then the mixture was stirred for 2 hours, TMSCl (1.89 g, 17.5 mmol) was added dropwise at −78° C. After stirring for another 2 hours, Br₂ (1.92 g, 12 mmol) was added dropwise. The mixture was stirred for an hour at that temperature and 30 minutes at 0° C. The mixture was quenched with aq.HCl, extracted with EtOAc (30 mL×3). The combined organic layers were washed with brine, dried over Na₂SO₄, concentrated and purified by column chromatography (petroleum ether/EtOAc=5:1˜1:1) to give the target compound (1.9 g, 56%) as colorless oil. MS: M/e 336/338 (M+1)⁺.

### Step B: tert-butyl 4-(1-(5-amino-2-(furan-2-yl)-7H-pyrazolo[4,3-e][1,2,4]triazolo[1,5-c]pyrimidin-7-yl)-2-methoxy-2-oxoethyl)piperidine-1-carboxylate

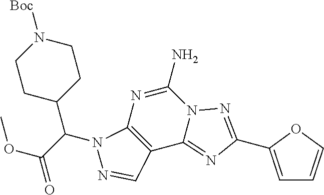 [SMILES: COC(=O)C(C1CCN(C(=O)OC(C)(C)C)CC1)n1ncc2c1nc(N)n1nc(-c3ccco3)nc21]

To a stirred solution of 2-(furan-2-yl)-7H-pyrazolo[4,3-e][1,2,4]triazolo[1,5-c]pyrimidin-5-amine (482 mg, 2 mmol) in DMF (5 mL) was added K₂CO₃ (552 mg, 4 mmol), then the product of step A (806.4 mg, 2.4 mmol) was added. After the addition, the reaction mixture was stirred for 2 days. The mixture was poured into H₂O (15 mL) and extracted with EtOAc (30 mL×3). The combined organic layers were washed with brine, dried over Na₂SO₄, concentrated and purified by column chromatography (petroleum ether/EtOAc=4:1˜1:1) to give the target compound (168 mg, 16.9%) as a white solid. MS: M/e 497 (M+1)⁺.

### Step C: 2-(5-amino-2-(furan-2-yl)-7H-pyrazolo[4,3-e][1,2,4]triazolo[1,5-c]pyrimidin-7-yl)-2-(1-(tert-butoxycarbonyl)piperidin-4-yl)acetic Acid

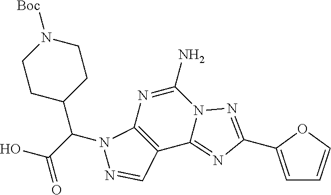 [SMILES: CC(C)(C)OC(=O)N1CCC(C(C(=O)O)n2ncc3c2nc(N)n2nc(-c4ccco4)nc32)CC1]

To a stirred solution of the product of step B (168 mg, 0.338 mmol) in MeOH (5 mL) was added aq.NaOH (2 M, 2 mL). After the addition, the reaction was stirred overnight. Most MeOH was removed to give aqueous layer, which was acidified to pH=3˜4 and filtered. The cake was collected, dried to give the target compound (150 mg, 92%) as a white solid. MS: M/e 483 (M+1)⁺.

### Step D: tert-butyl 4-(1-(5-amino-2-(furan-2-yl)-7H-pyrazolo[4,3-e][1,2,4]triazolo[1,5-c]pyrimidin-7-yl)-2-(4-(4-(2-methoxyethoxy)phenyl)piperazin-1-yl)-2-oxoethyl)piperidine-1-carboxylate

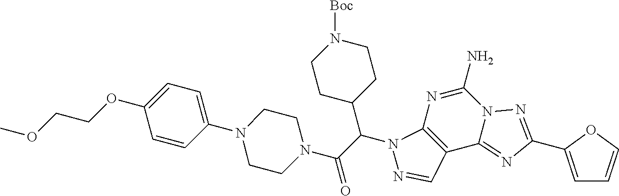 [SMILES: COCCOc1ccc(N2CCN(C(=O)C(C3CCN(C(=O)OC(C)(C)C)CC3)n3ncc4c3nc(N)n3nc(-c5ccco5)nc43)CC2)cc1]

A mixture of the product of C (336 mg, 0.72 mmol), 1-(4-(2-methoxyethoxy)phenyl)piperazine (170 mg, 0.72 mmol), HATU (330 mg, 0.864 mmol) and DIPEA (186 mg, 1.44 mmol) in DMF (3 mL) was stirred overnight. The mixture was poured into H₂O (15 mL) and extracted with EtOAc (10 mL×3). The combined organic layers were washed with brine, dried over Na₂SO₄, concentrated and purified by column chromatography (petroleum ether/EtOAc=2:1˜100% EtOAc) to give crude product, which was further purified by Pre-TLC (EtOAc) to give the target compound (30 mg, 6%) as a white solid. MS: M/e 701 (M+1)⁺.

### Step E: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(piperidin-4-yl)ethan-1-one

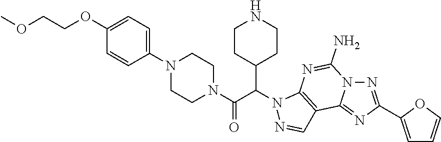 [SMILES: COCCOc1ccc(N2CCN(C(=O)C(C3CCNCC3)n3ncc4c3nc(N)n3nc(-c5ccco5)nc43)CC2)cc1]

To a stirred solution of the product of step D (30 mg, 0.043 mmol) in CH₂Cl₂ (3 mL) was added HCl (g)/EtOAc (4.0 M, 1 mL). After the addition, the mixture was stirred overnight. The mixture was concentrated to give the target compound (20 mg, 70%). ¹H NMR (400 MHz, DMSO-d6) δ 8.28 (s, 1H), 8.24 (s, 2H), 7.95 (s, 1H), 7.52 (s, 2H), 7.23 (d, J=2.8 Hz, 1H), 6.99 (d, J=8.4 Hz, 2H), 6.73 (s, 1H), 5.37 (d, J=9.6 Hz, 1H), 4.16-3.74 (m, 6H), 3.61 (s, 2H), 3.44 (m, 2H), 3.27 (s, 5H), 3.14 (m, 1H), 3.02-2.71 (m, 4H), 2.07-1.97 (m, 1H), 1.71-1.54 (m, 1H), 1.47-1.29 (m, 1H), 1.07 (m, 1H), 0.91 (m, 1H) ppm. MS: M/e 601 (M+1)⁺.

### Compound A59: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)-2,6-dimethylpiperazin-1-yl)-2-phenylethan-1-one

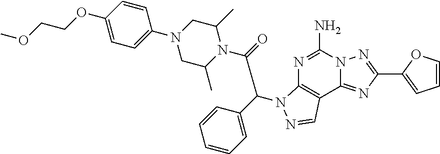 [SMILES: COCCOc1ccc(N2CC(C)N(C(=O)C(c3ccccc3)n3ncc4c3nc(N)n3nc(-c5ccco5)nc43)C(C)C2)cc1]

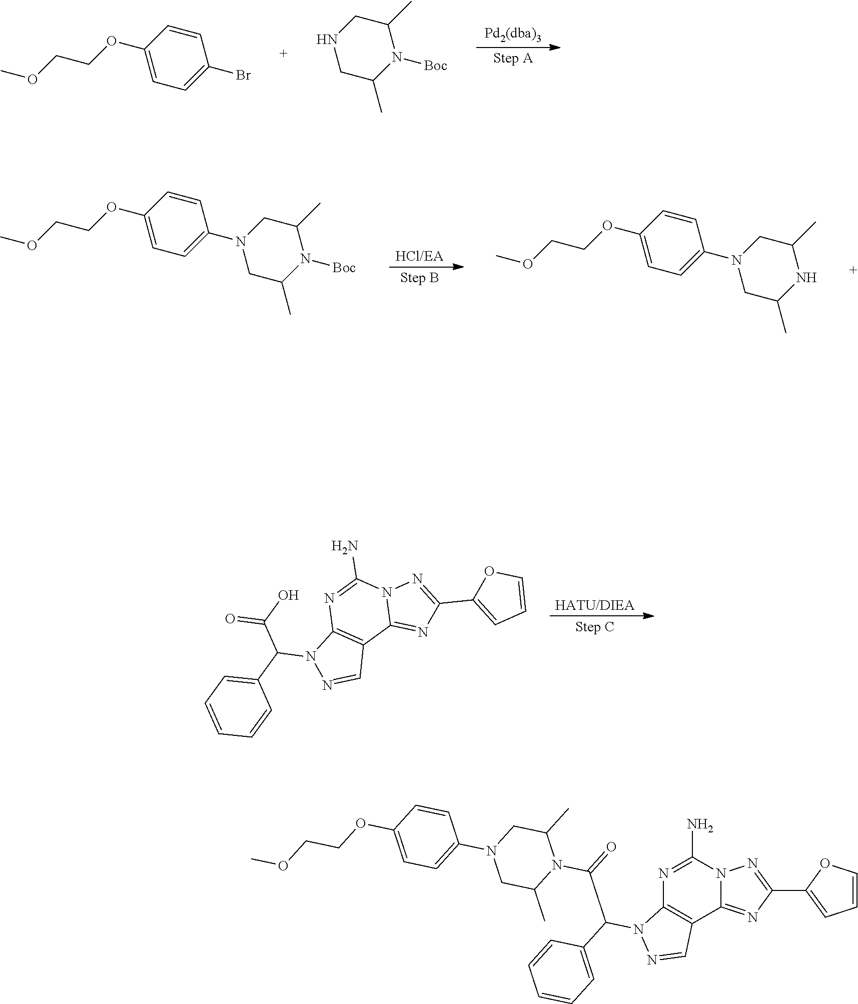

### Step A: tert-butyl 4-(4-(2-methoxyethoxy)phenyl)-2,6-dimethylpiperazine-1-carboxylate

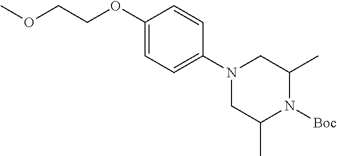 [SMILES: COCCOc1ccc(N2CC(C)N(C(=O)OC(C)(C)C)C(C)C2)cc1]

A mixture of 1-bromo-4-(2-methoxyethoxy)benzene (1 g, 4.3 mmol), tert-butyl 2,6-dimethylpiperazine-1-carboxylate (930 mg, 4.3 mmol), X-Phos (409 mg, 0.76 mmol), Pd₂(dba)₃ (394 mg, 0.4 mmol) and Cs₂CO₃ (2.8 g, 8.6 mmol) in toluene (20 mL) was heated at 95° C. overnight. The solvent was evaporated. The residue was added with water (10 mL), extracted with ethyl acetate (20 mL) and purified by column chromatography (PE:EA=10:1) to get the product (1.1 g, 70%). MS: M/e 365 (M+1)⁺

### Step B: 1-(4-(2-methoxyethoxy)phenyl)-3,5-dimethylpiperazine

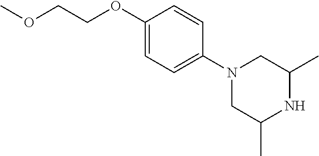

A solution of HCl in ethyl acetate (4 M, 10 mL) was added to tert-butyl 4-(4-(2-methoxyethoxy)phenyl)-2,6-dimethylpiperazine-1-carboxylate (1.1 g, 3 mmol) in a flask. The reaction mixture was stirred at rt overnight. The solution was concentrated, added with water (10 mL), basified with NaHCO₃ solution and extracted with ethyl acetate (10 mL). The organic layer was dried and concentrated to obtain the product, which was used in the next step directly. MS: M/e 265 (M+1)⁺

### Step C: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)-2,6-dimethylpiperazin-1-yl)-2-phenylethan-1-one

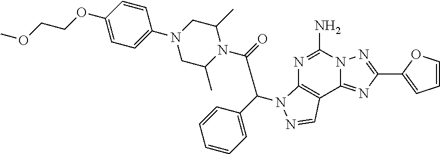 [SMILES: COCCOc1ccc(N2CC(C)N(C(=O)C(c3ccccc3)n3ncc4c3nc(N)n3nc(-c5ccco5)nc43)C(C)C2)cc1]

A mixture of 2-(5-amino-2-(furan-2-yl)-7H-pyrazolo[4,3-e][1,2,4]triazolo[1,5-c]pyrimidin-7-yl)-2-phenylacetic acid (50 mg, 0.13 mmol), 1-(4-(2-methoxyethoxy)phenyl)-3,5-dimethylpiperazine (48 mg, 0.16 mmol), HATU (61 mg, 0.16 mmol) and DIEA (33 mg, 0.26 mmol) in DMF (5 mL) was stirred at rt for 2 hrs. The solution was added with water (10 mL), extracted with ethyl acetate (10 mL) and washed with brine (10 mL). The organic layer was dried, concentrated and purified by column chromatography (PE:EA=1:1) to get the product (25 mg, 29%). ¹H NMR (400 MHz, DMSO-d6) δ 8.19 (d, J=4.0 Hz, 1H), 8.13 (br.s, 2H), 7.95 (br.s, 1H), 7.41-7.37 (m, 5H), 7.26-7.24 (m, 1H), 6.87-6.81 (m, 5H), 6.74 (br.s, 1H), 4.60 (s, 1H), 4.01-3.98 (m, 3H), 3.62-3.60 (t, J=4.0 Hz, 2H), 3.29 (s, 3H), 3.22 (d, J=8.0 Hz, 1H), 2.69-2.43 (m, 2H), 1.30-1.24 (m, 5H), 1.04 (d, J=8.0 Hz, 1H), 0.94-0.91 (m, 1H) ppm. MS: M/e 622 (M+1)⁺

### Compound A60: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(1-methylpiperidin-4-yl)ethan-1-one

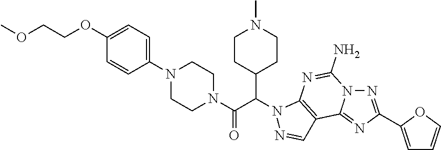 [SMILES: COCCOc1ccc(N2CCN(C(=O)C(C3CCN(C)CC3)n3ncc4c3nc(N)n3nc(-c5ccco5)nc43)CC2)cc1]

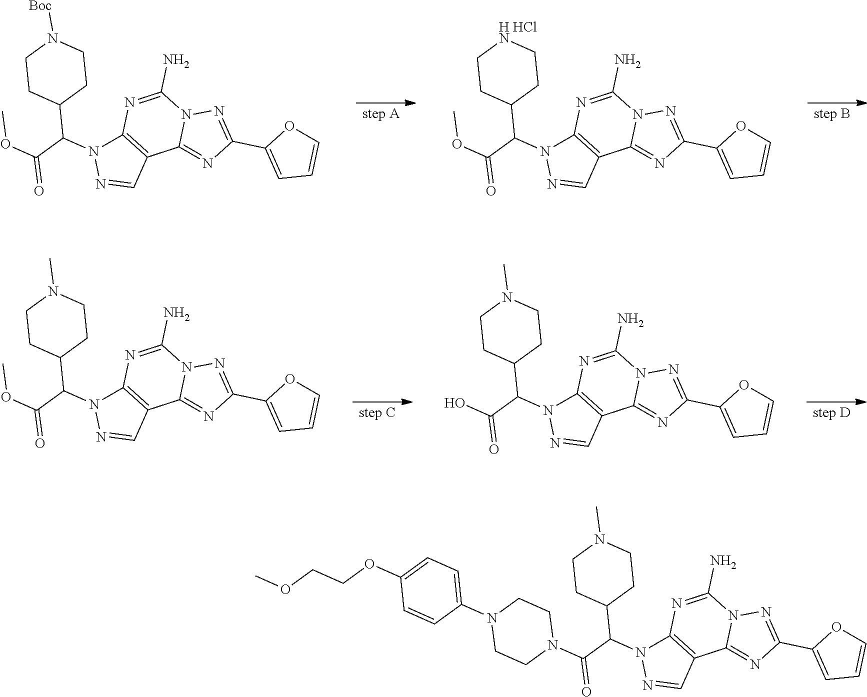

### Step A: methyl 2-(5-amino-2-(furan-2-yl)-7H-pyrazolo[4,3-e][1,2,4]triazolo[1,5-c]pyrimidin-7-yl)-2-(piperidin-4-yl)acetate Hydrochloride

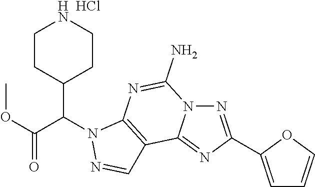 [SMILES: Cl ; COC(=O)C(C1CCNCC1)n1ncc2c1nc(N)n1nc(-c3ccco3)nc21]

To a stirred solution of tert-butyl 4-(1-(5-amino-2-(furan-2-yl)-7H-pyrazolo[4,3-e][1,2,4]triazolo[1,5-c]pyrimidin-7-yl)-2-methoxy-2-oxoethyl)piperidine-1-carboxylate (246 mg, 0.496 mmol) in CH₂Cl₂ (10 mL) was added HCl (g)/EtOAc (4.0 M, 4 mL). After the addition, the reaction mixture was stirred overnight. The reaction mixture was concentrated in vacuo to give the target compound (196 mg, 100%), which was directly used to the next step. MS: M/e 397 (M+1)⁺.

### Step B: methyl 2-(5-amino-2-(furan-2-yl)-7H-pyrazolo[4,3-e][1,2,4]triazolo[1,5-c]pyrimidin-7-yl)-2-(1-methylpiperidin-4-yl)acetate

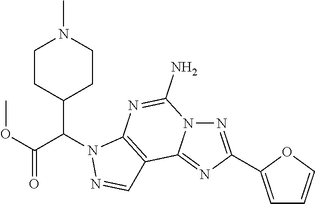 [SMILES: COC(=O)C(C1CCN(C)CC1)n1ncc2c1nc(N)n1nc(-c3ccco3)nc21]

To a stirred solution of the product of step A (196 mg, 0.496 mmol) in MeOH (5 mL) was added aq.HCHO (37%, 2 mL) and AcOH (1 mL), then the mixture was stirred for 30 minutes, then NaBH₃CN (62.5 mg, 0.992 mmol) was added and stirred for 2 hours. Most MeOH was removed to give the residue, then extracted with EtOAc (20 mL). The organic layer was discarded and the aqueous layer was basified to pH=11˜12 with K₂CO₃, then extracted with EtOAc (20 mL×3). The combined organic layers were washed with brine, dried over Na₂SO₄, concentrated to give the target compound (165 mg, 81.1%) as a white solid. MS: M/e 411 (M+1)⁺.

### Step C: 2-(5-amino-2-(furan-2-yl)-7H-pyrazolo[4,3-e][1,2,4]triazolo[1,5-c]pyrimidin-7-yl)-2-(1-methylpiperidin-4-yl)acetic acid

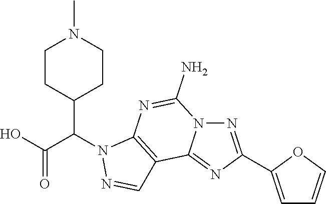 [SMILES: CN1CCC(C(C(=O)O)n2ncc3c2nc(N)n2nc(-c4ccco4)nc32)CC1]

To a stirred solution of the product of step B (165 mg, 0.402 mmol) in MeOH (5 mL) was added aq.NaOH (2.0 M, 2 mL). After the addition, the mixture was stirred overnight. The mixture was acidified to pH=5˜6 with aq.HCl, then concentrated to give the residue, which was washed with CH₂Cl₂/MeOH (10 mL/2 mL) and filtered. The filtered was concentrated to give the target compound (133 mg, 83.5%) as a white solid. MS: M/e 397 (M+1)⁺.

### Step D: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(1-methylpiperidin-4-yl)ethan-1-one

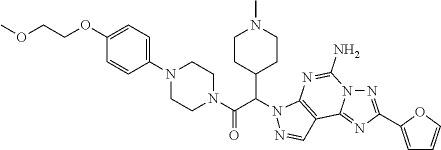 [SMILES: COCCOc1ccc(N2CCN(C(=O)C(C3CCN(C)CC3)n3ncc4c3nc(N)n3nc(-c5ccco5)nc43)CC2)cc1]

A mixture of the product of step C (133 mg, 0.336 mmol), 1-(4-(2-methoxyethoxy)phenyl)piperazine (80 mg, 0.336 mmol), HATU (154 mg, 0.403 mmol) and DIPEA (87 mg, 0.672 mmol) in DMF (4 mL) was stirred for 2 hours. The mixture was poured into H₂O (15 mL) and filtered. The cake was collected, dried and purified by prep-HPLC to give the target compound (25 mg, 12%). ¹H NMR (400 MHz, DMSO-d6) δ 8.22 (s, 3H), 7.95 (s, 1H), 7.23 (d, J=2.8 Hz, 1H), 6.87-6.67 (m, 5H), 5.29 (d, J=9.6 Hz, 1H), 3.97 (m, 2H), 3.79-3.74 (m, 1H), 3.65-3.58 (m, 4H), 3.48 (m, 1H), 3.27 (s, 3H), 3.06-2.92 (m, 4H), 2.73 (m, 2H), 2.32-2.24 (m, 4H), 1.91-1.83 (m, 1H), 1.45-1.09 (m, 3H), 0.96-0.80 (m, 1H) ppm. MS: M/e 615 (M+1)⁺.

### Compound A61: 2-(5-amino-2-(furan-2-yl)-7H-pyrazolo[4,3-e][1,2,4]triazolo[1,5-c]pyrimidin-7-yl)-1-(4-(4-(2-hydroxyethoxy)phenyl)piperazin-1-yl)-2-phenylethan-1-one

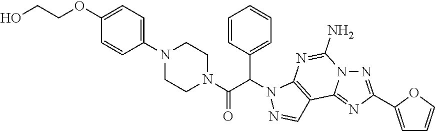 [SMILES: Nc1nc2c(cnn2C(C(=O)N2CCN(c3ccc(OCCO)cc3)CC2)c2ccccc2)c2nc(-c3ccco3)nn12]

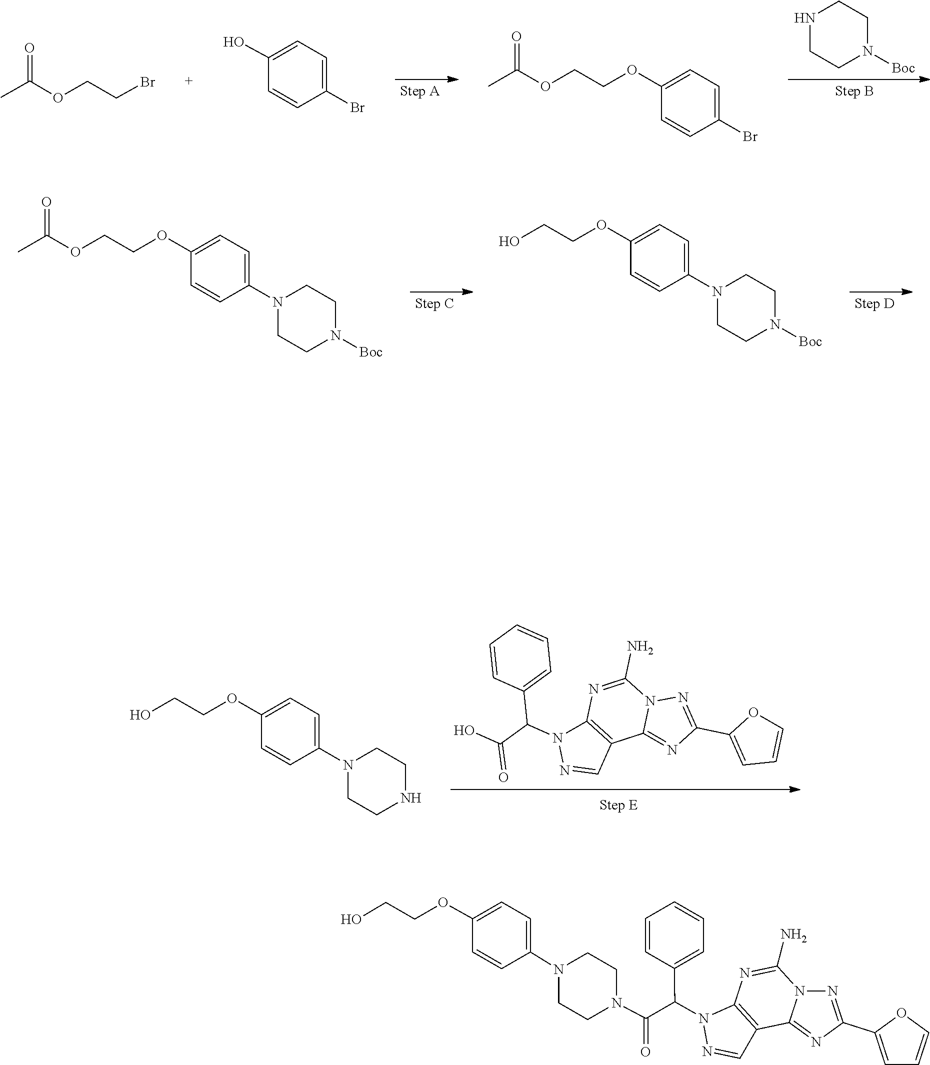

### Step A: 2-(4-bromophenoxy)ethyl Acetate

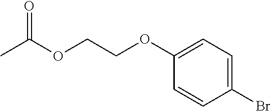

A mixture of 2-bromoethyl acetate (5.8 g, 34.9 mmol), 4-bromophenol (5.0 g, 29.1 mmol), and K₂CO₃ (8.0 g, 58.0 mmol) in DMF (50 mL) was stirred at 60° C. for 24 hrs. The mixture was filtered and the filtrate was diluted with 100 mL of EA, washed with brine (50 mL×3), dried over Na₂SO₄. The resulting residue was purified by column chromatography to give the title product (6.2 g, 69%) as a light yellow oil. MS: M/e 259, 261 (M+1)⁺.

### Step B: tert-butyl 4-(4-(2-acetoxyethoxy)phenyl)piperazine-1-carboxylate

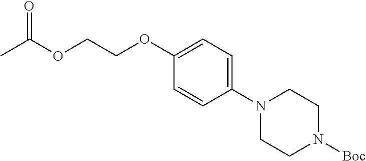 [SMILES: CC(=O)OCCOc1ccc(N2CCN(C(=O)OC(C)(C)C)CC2)cc1]

A mixture of 2-(4-bromophenoxy)ethyl acetate (2.0 g, 7.7 mmol), tert-butyl piperazine-1-carboxylate (2.0 g, 10.7 mmol), Pd₂(dba)₃ (600 mg, 0.65 mmol), X-phos (650 mg, 1.36 mmol) and Cs₂CO₃ (5.5 g, 16.9 mmol) in toluene (20 mL) was stirred at 90° C. under N₂ for 16 hrs. The mixture was filtered through a celite pad and the filtrate was concentrated and purified by column chromatography to give the title product (1.85 g, 66%) as a brown oil. MS: M/e 365 (M+1)⁺.

### Step C: tert-butyl 4-(4-(2-hydroxyethoxy)phenyl)piperazine-1-carboxylate

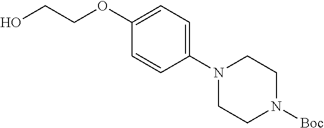 [SMILES: CC(C)(C)OC(=O)N1CCN(c2ccc(OCCO)cc2)CC1]

To a solution of tert-butyl 4-(4-(2-acetoxyethoxy)phenyl)piperazine-1-carboxylate (600 mg, 1.65 mmol) in MeOH (10 mL) was added aqueous solution of NaOH (2 M, 5 mL) at rt and stirred for 5 hrs. 20 mL of EA was added and the resulting solution was washed with brine (20 mL×3), dried over Na₂SO₄ and concentrated to give the title product (280 mg, crude) as a light yellow oil. MS: M/e 323 (M+1)⁺.

### Step D: 2-(4-(piperazin-1-yl)phenoxy)ethan-1-ol

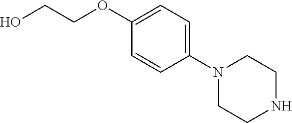 [SMILES: OCCOc1ccc(N2CCNCC2)cc1]

To a stirred solution of tert-butyl 4-(4-(2-hydroxyethoxy)phenyl)piperazine-1-carboxylate (280 mg, 0.87 mmol) in CH₂Cl₂ (10 mL) was added CF₃COOH (3 mL) at rt and the resulting solution was stirred at rt for 5 hrs. The mixture was concentrated to dryness. 5 mL of aqueous solution of NaHCO₃ was added and the resulting mixture was extracted with CH₂Cl₂ (5 mL×3). The combined organics were washed with brine (5 mL×3), dried over Na₂SO₄ and concentrated to give the title product (120 mg, crude) as a light yellow oil. MS: M/e 223 (M+1)⁺.

### Step E: 2-(5-amino-2-(furan-2-yl)-7H-pyrazolo[4,3-e][1,2,4]triazolo[1,5-c]pyrimidin-7-yl)-1-(4-(4-(2-hydroxyethoxy)phenyl)piperazin-1-yl)-2-phenylethan-1-one

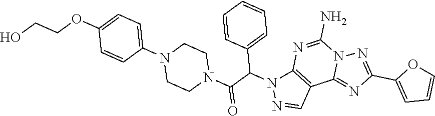 [SMILES: Nc1nc2c(cnn2C(C(=O)N2CCN(c3ccc(OCCO)cc3)CC2)c2ccccc2)c2nc(-c3ccco3)nn12]

To a mixture of 2-(4-(piperazin-1-yl)phenoxy)ethan-1-ol (40 mg, 0.18 mmol), 2-(5-amino-2-(furan-2-yl)-7H-pyrazolo[4,3-e][1,2,4]triazolo[1,5-c]pyrimidin-7-yl)-2-phenylacetic acid (50 mg, 0.13 mmol), DIEA (70 mg, 0.54 mmol) in DMF (2 mL) was added HATU (65 mg, 0.17 mmol) at rt and the mixture was stirred at rt for 16 hrs. 10 mL of EA was added and the mixture was washed with brine (10 mL×3), dried over Na₂SO₄ and concentrated. The resulting residue was purified by prep-TLC (CH₂Cl₂/EA=1:2) to give the title product (9.5 mg, yield: 9%). ¹H NMR (400 MHz, DMSO-d6) δ 8.28-8.05 (m, 3H), 7.95 (s, 1H), 7.47-7.29 (m, 5H), 7.24 (d, J=3.2 Hz, 1H), 6.92-6.76 (m, 5H), 6.75-6.71 (m, 1H), 3.87 (t, J=4.8 Hz, 2H), 3.79-3.52 (m, 6H), 3.02-2.89 (m, 2H), 2.68-2.58 (m, 2H). MS: M/e 580 (M+1)⁺.

### Compound A62: 2-(5-amino-2-(furan-2-yl)-7H-pyrazolo[4,3-e][1,2,4]triazolo[1,5-c]pyrimidin-7-yl)-1-(4-(4-(2-hydroxypropoxy)phenyl)piperazin-1-yl)-2-phenylethan-1-one

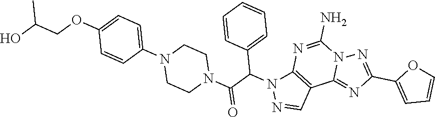 [SMILES: CC(O)COc1ccc(N2CCN(C(=O)C(c3ccccc3)n3ncc4c3nc(N)n3nc(-c5ccco5)nc43)CC2)cc1]

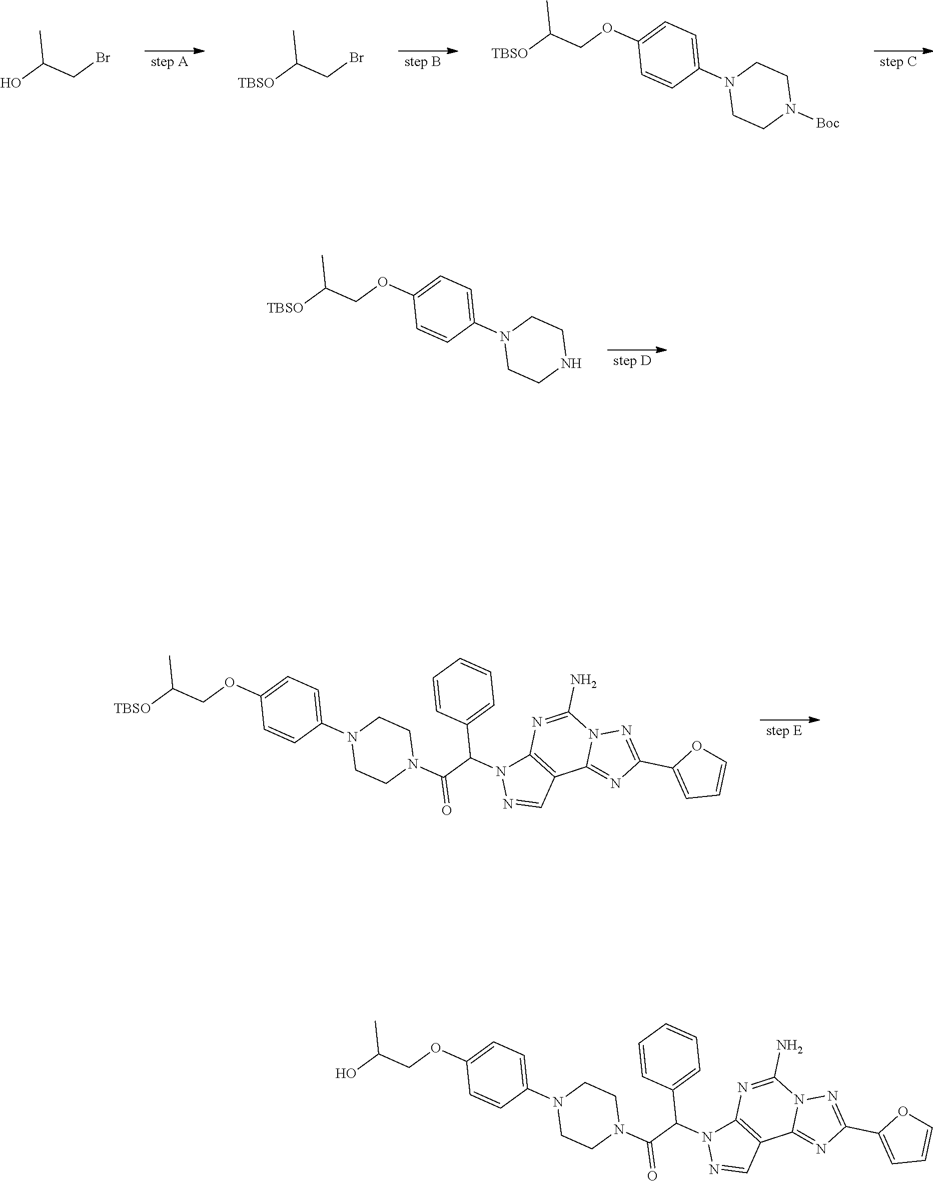

### Step A: ((1-bromopropan-2-yl)oxy)(tert-butyl)dimethylsilane

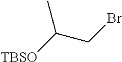

To a stirred solution of 1-bromopropan-2-ol (1.39 g, 10 mmol) in CH₂Cl₂ (20 mL) was added 1H-imidazole (0.8 g, 1.2 mmol), then TBSCl (1.8 g, 1.2 mmol) was added. After the addition, the reaction was stirred overnight. The mixture was washed with H₂O, brine, dried over Na₂SO₄ and concentrated in vacuo to give the target compound (2.1 g, 83%) as colorless oil.

### Step B: tert-butyl 4-(4-(2-((tert-butyldimethylsilyl)oxy)propoxy)phenyl)piperazine-1-carboxylate

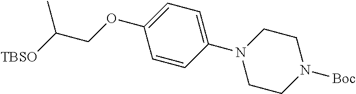 [SMILES: CC(COc1ccc(N2CCN(C(=O)OC(C)(C)C)CC2)cc1)O[Si](C)(C)C(C)(C)C]

A mixture of the product of step A (500 mg, 1.97 mmol), tert-butyl 4-(4-hydroxyphenyl)piperazine-1-carboxylate (547 mg, 1.97 mmol) and K₂CO₃ (544 mg, 3.94 mmol) in DMF (5 mL) was stirred at 60° C. overnight. The mixture was poured into H₂O (10 mL) and extracted with EtOAc (10 mL×3). The combined organic layers were washed with brine, dried over Na₂SO₄ and concentrated in vacuo to give the residue, which was purified by column chromatography (petroleum ether/EtOAc=5:1˜1:1) to give the target compound (200 mg, 44.4%) as yellow oil. MS: M/e 451 (M+1)⁺.

### Step C: 1-(4-(2-((tert-butyldimethylsilyl)oxy)propoxy)phenyl)piperazine

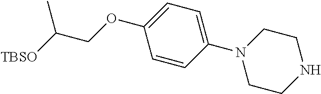 [SMILES: CC(COc1ccc(N2CCNCC2)cc1)O[Si](C)(C)C(C)(C)C]

To a stirred solution of the product of step B (80 mg, 0.177 mmol) in CH₂Cl₂ (10 mL) was added TFA (1 mL). After stirring for 2 hours, the mixture was basified to pH=10˜12 with K₂CO₃ and extracted with CH₂Cl₂ (20 mL×3). The combined organic layers were washed with brine, dried over Na₂SO₄, concentrated to give the target compound (crude product, 100%), which was directly used to the next step. MS: M/e 351 (M+1)⁺.

### Step D: 2-(5-amino-2-(furan-2-yl)-7H-pyrazolo[4,3-e][1,2,4]triazolo[1,5-c]pyrimidin-7-yl)-1-(4-(4-(2-((tert-butyldimethylsilyl)oxy)propoxy)phenyl)piperazin-1-yl)-2-phenylethan-1-one

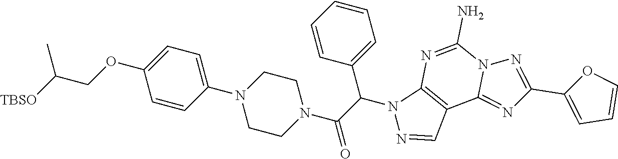 [SMILES: CC(COc1ccc(N2CCN(C(=O)C(c3ccccc3)n3ncc4c3nc(N)n3nc(-c5ccco5)nc43)CC2)cc1)O[Si](C)(C)C(C)(C)C]

A mixture of 2-(5-amino-2-(furan-2-yl)-7H-pyrazolo[4,3-e][1,2,4]triazolo[1,5-c]pyrimidin-7-yl)-2-phenylacetic acid (66 mg, 0.177 mmol), the product of step C (0.177 mmol), HATU (81 mg, 0.212 mmol) and DIPEA (45 mg, 0.354 mmol) in DMF (4 mL) was stirred overnight. The reaction mixture was poured into H₂O (15 mL), then extracted with EtOAc (15 mL×3). The combined organic layers were washed with brine, dried over Na₂SO₄, concentrated and purified by column chromatography (petroleum ether/EtOAc=3:1˜100% EtOAc) to give the target compound (20 mg, 20%) as a white solid. MS: M/e 708 (M+1)⁺.

### Step E: 2-(5-amino-2-(furan-2-yl)-7H-pyrazolo[4,3-e][1,2,4]triazolo[1,5-c]pyrimidin-7-yl)-1-(4-(4-(2-hydroxypropoxy)phenyl)piperazin-1-yl)-2-phenylethan-1-one

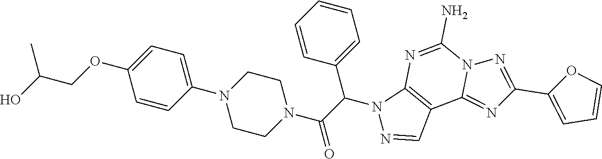 [SMILES: CC(O)COc1ccc(N2CCN(C(=O)C(c3ccccc3)n3ncc4c3nc(N)n3nc(-c5ccco5)nc43)CC2)cc1]

To a stirred solution of the product of step D (20 mg, 0.028 mmol) in THF (10 mL) was added TBAF (20 mg). After the addition, the reaction mixture was stirred for 2 days. The reaction mixture was poured into H₂O (20 mL) and extracted with EtOAc (10 mL×3). The combined organic layers were washed with brine, dried over Na₂SO₄, concentrated to give the residue, which was purified by prep-HPLC to give the target compound (10 mg) as TFA salt. ¹H NMR (400 MHz, DMSO-d6) δ 8.20 (s, 1H), 8.15 (s, 2H), 7.95 (s, 1H), 7.43-7.30 (m, 5H), 7.24-7.23 (d, J=3.2 Hz, 1H), 6.86 (s, 1H), 6.79 (m, 4H), 6.74 (s, 1H), 3.88 (m, 1H), 3.76-3.70 (m, 2H), 2.96 (m, 4H), 2.67 (m, 2H), 2.04-1.94 (m, 1H), 1.56-1.46 (m, 2H), 1.12-1.10 (d, J=6.0 Hz, 3H) ppm. MS: M/e 594 (M+1)⁺.

### Intermediate-I: 2-(5-amino-2-(furan-2-yl)-7H-pyrazolo[4,3-e][1,2,4]triazolo[1,5-c]pyrimidin-7-yl)-2-phenylpropanoic Acid

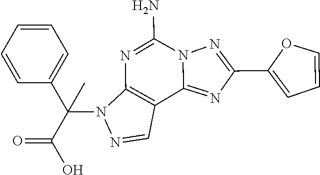 [SMILES: CC(C(=O)O)(c1ccccc1)n1ncc2c1nc(N)n1nc(-c3ccco3)nc21]

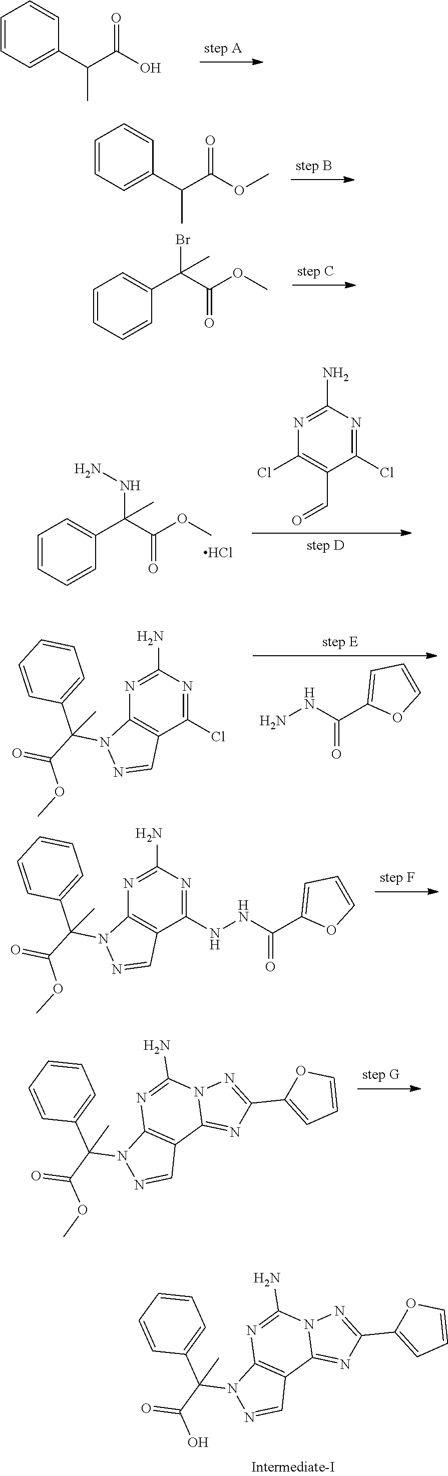

### Step A: methyl 2-phenylpropanoate

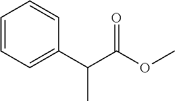

To a solution of 2-phenylpropanoic acid (5 g, 33.33 mmol) in MeOH (15 mL), sulfoxide chloride (5.15 g, 50 mmol) was added dropwise at 0° C. After the addition, the reaction mixture was stirred at rt for 3 h. The mixture was concentrated, quenched with ice water (20 mL), extracted with EtOAc (30 mL×3). The combined organic layers were washed with brine, dried over Na₂SO₄, concentrated and purified by column chromatography (petroleum ether/EtOAc=20:1˜5:1) to give methyl 2-phenylpropanoate (5.22 g, 95.49%) as yellow oil. MS: M/e 165 (M+1)⁺.

### Step B: methyl 2-bromo-2-phenylpropanoate

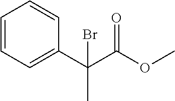

A mixture of methyl 2-phenylpropanoate (5.22 g, 31.83 mmol), NBS (6.80 g, 38.19 mmol), BPO (0.385 g, 1.591 mmol) in carbon tetrachloride (20 mL) was stirred at 70° C. overnight. The mixture was concentrated, the residue was washed with PE and filtered, the filtrate was concentrated to give methyl 2-bromo-2-phenylpropanoate (7.57 g, 97.87%) as yellow oil. 1H NMR (400 MHz, CDCl3) δ 7.55 (d, J=7.5 Hz, 2H), 7.40-7.27 (m, 3H), 3.80 (s, 3H), 2.30 (s, 3H)

### Step C: methyl 2-hydrazinyl-2-phenylpropanoate Hydrochloride

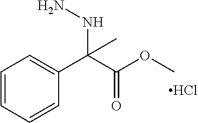

To a stirred solution of methyl 2-bromo-2-phenylpropanoate (15 g, 62.2 mmol) in acetonitrile (200 mL) was added hydrazine hydrate (80%, 15.5 g, 249 mmol). After addition, the reaction mixture was warmed up to 50° C. and stirred for 3.5 h. The reaction mixture was concentrated in vacuo. The residue was added with H₂O (300 mL) and extracted with EA (200 mL×3). The combined organic layers were washed with brine, dried over Na₂SO₄ and concentrated in vacuo. The residue was added with EA (50 mL) and added to 4M HCl/EA solution (200 mL) at 0° C. After addition, the mixture was stirred for 1 h at 0° C., then the mixture was filtered and the filter cake was washed with EA (50 mL), dried to give the target product as a white solid (9.8 g, 68.5%). 1H NMR (400 MHz, DMSO-d6) δ 9.28 (s, 2H), 7.43-7.38 (m, 5H), 6.23 (s, 1H), 3.73 (s, 3H), 1.72 (s, 3H) ppm. MS: M/e 195 (M+1)⁺.

### Step D: methyl 2-(6-amino-4-chloro-1H-pyrazolo[3,4-d]pyrimidin-1-yl)-2-phenylpropanoate

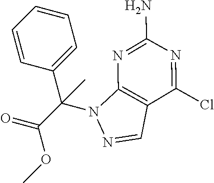 [SMILES: COC(=O)C(C)(c1ccccc1)n1ncc2c(Cl)nc(N)nc21]

To a stirred mixture of methyl 2-hydrazinyl-2-phenylpropanoate hydrochloride (2.38 g, 10.3 mmol) in acetonitrile (30 mL) was added 2-amino-4,6-dichloropyrimidine-5-carbaldehyde (1.96 g, 10.3 mmol). After addition, the reaction mixture was stirred at rt overnight. The reaction mixture was warmed up to 70° C. and stirred for 2 h. 1M NaHCO₃ aqueous solution was added to the mixture and the mixture was extracted with EA (50 mL×3). The combined organic phase was dried with Na₂SO₄ and concentrated in vacuo. The crude product was purified by column chromatography (petroleum ether/EA=4:1) to give the target compound (2.2 g, 64.3%) as a white solid. 1H NMR (400 MHz, DMSO-d6) δ 8.05 (s, 1H), 7.35-7.31 (m, 5H), 7.13-7.11 (m, 2H), 3.71 (s, 3H), 3.25 (s, 3H) ppm. MS: M/e 332 (M+1)⁺.

### Step E: methyl 2-(6-amino-4-(2-(furan-2-carbonyl)hydrazinyl)-1H-pyrazolo[3,4-d]pyrimidin-1-yl)-2-phenylpropanoate

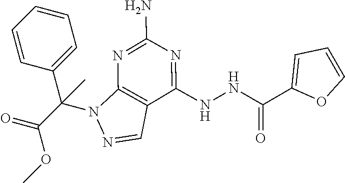 [SMILES: COC(=O)C(C)(c1ccccc1)n1ncc2c(NNC(=O)c3ccco3)nc(N)nc21]

To a stirred mixture of methyl 2-(6-amino-4-chloro-1H-pyrazolo[3,4-d]pyrimidin-1-yl)-2-phenylpropanoate (2.03 g, 6.4 mmol) in DMSO (20 mL) was added furan-2-carbohydrazide (0.85 g, 6.7 mmol) and DIEA (2.36 g, 18.3 mmol). After addition, the reaction mixture was stirred at 120° C. overnight. Most of solvent was concentrated in vacuo and the residue was added with H₂O (20 mL) and stirred for 1 h. The mixture was filtered and the filter cake was washed with water. The crude product was dried at 40° C. and used directly in next step (850 mg, 31.5%). MS: M/e 422 (M+1)⁺.

### Step F: methyl 2-(5-amino-2-(furan-2-yl)-7H-pyrazolo[4,3-e][1,2,4]triazolo[1,5-c]pyrimidin-7-yl)-2-phenylpropanoate

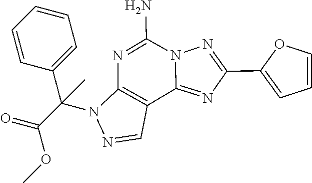 [SMILES: COC(=O)C(C)(c1ccccc1)n1ncc2c1nc(N)n1nc(-c3ccco3)nc21]

A mixture of methyl 2-(6-amino-4-(2-(furan-2-carbonyl)hydrazinyl)-1H-pyrazolo[3,4-d]pyrimidin-1-yl)-2-phenylpropanoate (750 mg, 1.78 mmol) in BSA (7.5 mL) and HMDS (7.5 mL) was stirred at 110° C. overnight. The mixture was concentrated in vacuo to remove BSA and HMDS. The residue was added with H₂O and extracted with EA (20 mL×3). The combined organic phase was washed with brine, dried with Na₂SO₄ and concentrated in vacuo. The crude product was purified by column chromatography (petroleum ether/EA=4:1-2:1) to give the target compound (150 mg, 20.9%) as a white solid. 1H NMR (400 MHz, CDCl₃) δ 8.31 (s, 1H), 7.64 (s, 1H), 7.31 (m, 5H), 7.21 (d, J=4.0 Hz, 1H), 6.59-6.58 (m, 1H), 3.76 (s, 3H), 2.4 (s, 3H) ppm. MS: M/e 404 (M+1)⁺.

### Step G: 2-(5-amino-2-(furan-2-yl)-7H-pyrazolo[4,3-e][1,2,4]triazolo[1,5-c]pyrimidin-7-yl)-2-phenylpropanoic Acid (Intermediate-I)

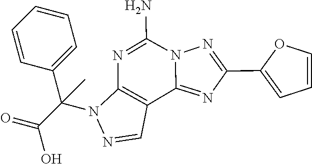 [SMILES: CC(C(=O)O)(c1ccccc1)n1ncc2c1nc(N)n1nc(-c3ccco3)nc21]

To a stirred mixture of methyl 2-(5-amino-2-(furan-2-yl)-7H-pyrazolo[4,3-e][1,2,4]triazolo[1,5-c]pyrimidin-7-yl)-2-phenylpropanoate (150 mg, 0.34 mmol) in EtOH (3 mL) was added aq. NaOH (2.0 M, 3 mL). After addition, the reaction mixture was stirred at 70° C. for 1 h. Most of EtOH was removed to give the aqueous layer, then acidified to pH=3˜4 with aq. HCl and filtered, the filter cake was collected, dried to give the target compound (130 mg, 98.3%) as a white solid. 1H NMR (400 MHz, DMSO-d6) δ 13.31 (s, 1H), 8.21 (s, 1H), 7.99 (s, 2H), 7.95 (s, 1H), 7.33-7.21 (m, 6H), 6.75-6.74 (m, 1H), 2.32 (s, 3H) ppm. MS: M/e 390 (M+1)+.

Intermediate-I was separated into two enantiomeric stereoisomers (Intermediate-Ia, earlier peak, and Intermediate-Ib, later peak) by chiral prep-HPLC. The chiral separation conditions are shown below.

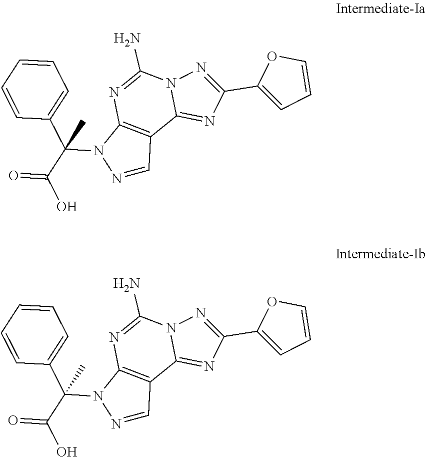 [SMILES: C[C@@](C(=O)O)(c1ccccc1)n1ncc2c1nc(N)n1nc(-c3ccco3)nc21 ; C[C@](C(=O)O)(c1ccccc1)n1ncc2c1nc(N)n1nc(-c3ccco3)nc21]

| Column | CHIRALPAK-IC |
|---|---|
| Column size | 5 cm × 15 cm, 5 um |
| Injection | 8 ml |
| Mobile phase | CO₂:MeOH(0.1% DEA) = 60:40 |
| Flow rate | 160 ml/min |
| Wavelength | UV 220 nm |
| Temperature | 25° C. |
| Sample solution | 15.1 mg/ml in EtOH:ACN:DCM = 1:1:1 |
| Prep-SFC equipment | Prep-SFC-350 |

Intermediate-Ib was synthesized using the following route: (R)-2-(5-amino-2-(furan-2-yl)-7H-pyrazolo[4,3-e][1,2,4]triazolo[1,5-c]pyrimidin-7-yl)-2-phenylpropanoic Acid

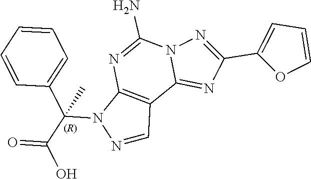

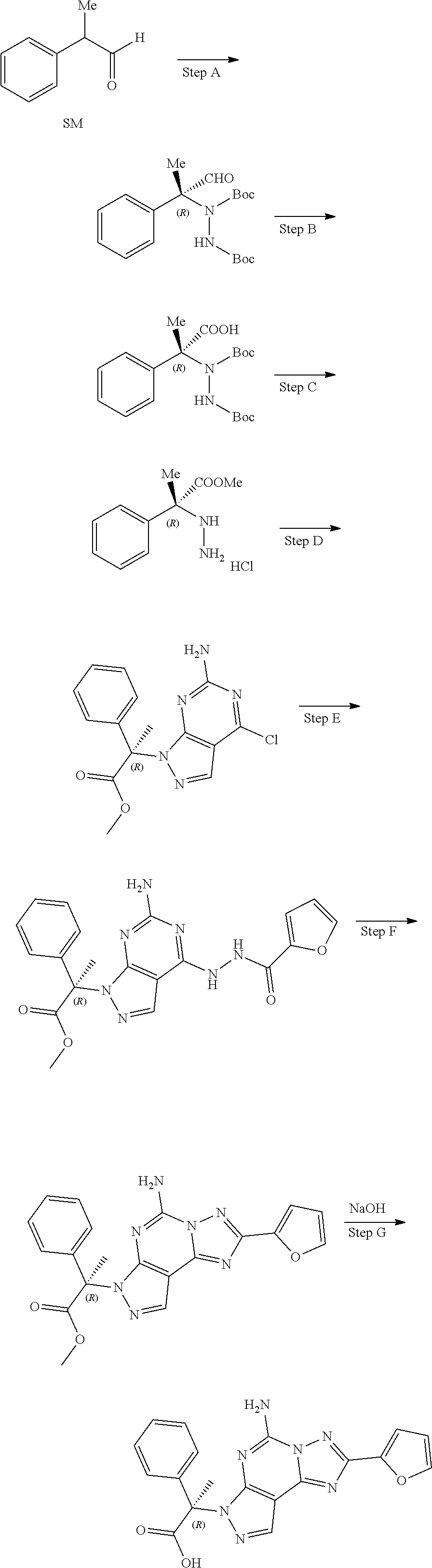

### Step A: di-tert-butyl (R)-1-(1-oxo-2-phenylpropan-2-yl)hydrazine-1,2-dicarboxylate

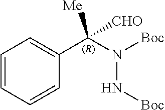

To a stirred solution of (S)-2-amino-4-(tert-butoxy)-4-oxobutanoic acid (15 g, 0.08 mol) in dry THF (0.8 L) were added 2-phenylpropanal (53.6 g, 0.4 mol) and di-tert-butyl azodicarbarboxylate (DTAD 92 g, 0.4 mol) at 0° C. After addition, the reaction was slowly warmed to rt and stirred overnight under N2. The mixture was diluted with water/brine, extracted with EA (600 mL). The combined organic layers were washed with brine, dried over Na₂SO₄, concentrated to give the target compound (160 g, crude) as yellow oil.

### Step B: (R)-2-(1,2-bis(tert-butoxycarbonyl)hydrazinyl)-2-phenylpropanoic Acid

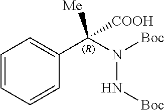

To a solution of the product of step A (120 g, 0.3 mol) in DCM (600 mL) was added a solution of NaH2PO4 (10.08 g, 0.084 mol) in water (150 mL). H₂O₂(150 mL, 1.46 mol, about 4.85 eq) was added at 0° C. Then a solution of 75% NaClO₂ (46 g, 0.51 mol, 1.275 eq) in water (300 mL) was added drop wise to keep the temperature below 10° C. After addition, the reaction was warmed to rt overnight. The stirring was stopped and the water layer was discarded. The organic layer was transferred into 10% NaHSO₃ solution (300 mL) and the resulting mixture was stirred at rt for one hour. The organic layer was collected and washed with 10% NaHSO₃ solution (300 mL), brine, dried over Na₂SO₄, filtered, and concentrated. To the residue was added CH₃CN (200 mL) and the mixture was concentrated again. The resulting residue was slurried with EA/PE (1/3˜200 mL) and stirred at rt overnight. The precipitate was filtered, washed with EA/PE (1/3, 100 mL) to give the target compound (47, 41% for two steps) as a white solid.

### Step C: methyl (R)-2-hydrazinyl-2-phenylpropanoate Hydrochloride

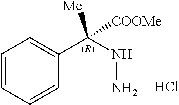

To a mixture of the product of step B (299 g, 0.787 mol) in MeOH (1.2 L) was added SOCl₂ (187 g, 1.57 mmol) dropwise at 10˜30° C. The resulting mixture was heated at 65° C. overnight. The mixture was cooled to rt and concentrated to give the residue, which was added with EA (100 mL) and concentrated again. The residue was slurry with EA (200 mL) and filtered, the cake was collected to give the target compound (176 g, 97%, ee %=99.3%) as a white solid.

### Step D: methyl (R)-2-(6-amino-4-chloro-1H-pyrazolo[3,4-d]pyrimidin-1-yl)-2-phenylpropanoate

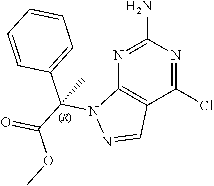

To a stirred mixture of the product of step C (11.5 g, 50 mmol) and 2-amino-4,6-dichloropyrimidine-5-carbaldehyde (9.6 g, 50 mmol) in CH₃CN (200 mL) was added POCl₃ (7.7 g, 50 mmol) at room temperature. After addition, the reaction mixture was stirred at 30° C. overnight. Then the reaction mixture was added dropwise to a system of EtOAc (200 mL)/aq. K₃PO₄ (37 g, 0.175 mol) in H₂O (200 mL) and stirred for 2 h. The organic layer was separated, washed with brine, dried over Na₂SO₄, concentrated to give the residue, which was dissolved in EtOAc (40 mL), active-carbon (5 g) was added and petroleum ether (80 mL) was added dropwise. The mixture was stirred for half an hour and filtered. The filtrate was concentrated to give the target compound (15 g, 90%) as a light yellow solid.

### Step E: methyl (R)-2-(6-amino-4-(2-(furan-2-carbonyl)hydrazinyl)-1H-pyrazolo[3,4-d]pyrimidin-1-yl)-2-phenylpropanoate

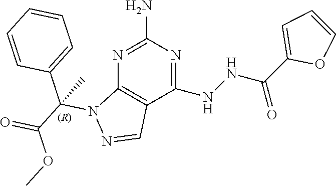

To a mixture of the product of step D (114 g, 0.344 mol) and furan-2-carbohydrazide (65 g, 0.5166 mol) in DMSO (115 mL) was added DIPEA (88 g, 0.6888 mol). The resulting mixture was heated at 80° C. overnight under N₂. The mixture was cooled to rt and diluted with PE. The PE layer was discarded. The DMSO layer was slowly add to water (1.1 L) with vigorous stirring. A suspension was formed, filtered and washed with water. The solid was slurry with water (500 mL) at rt for 2 h, filtered and washed with water, dried in the oven over 3 days to give the target compound (125 g, 86%) as brown solid.

### Step F: methyl (R)-2-(5-amino-2-(furan-2-yl)-7H-pyrazolo[4,3-e][1,2,4]triazolo[1,5-c]pyrimidin-7-yl)-2-phenylpropanoate

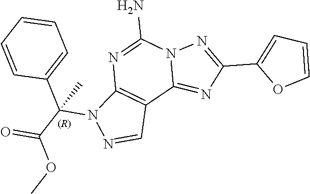

To a mixture of BSA (204 g, 1.072 mol) and HMDS (161 g, 1.072 mol) was added the product of step E (90.3 g, 0.2144 mol). The resulting mixture was heated at 115° C. for 5 hours. The mixture was cooled to rt and concentrated to dryness under reduced pressure. To the residue was added EtOH (200 mL) slowly at rt and the resulting mixture was heated at 75° C. for 1 hour. The suspension was cooled to rt, maintained at rt overnight with stirring, filtered and the cake was washed with PE, dried to give the target compound (66 g, 76%) as an off-white solid.

### Step G: (R)-2-(5-amino-2-(furan-2-yl)-7H-pyrazolo[4,3-e][1,2,4]triazolo[1,5-c]pyrimidin-7-yl)-2-phenylpropanoic Acid

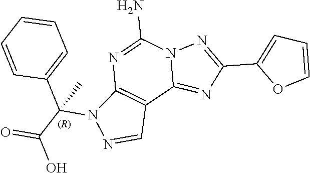

To a mixture of the product of step F (20.4 g, 50.62 mmol) in MeOH/THF (50 mL/150 mL) was added 4N NaOH (63 mL, 253 mmol). The resulting mixture was stirred at 30° C. (inner temperature 25˜30° C.) overnight. The solvent was concentrated under reduced pressure (bath temperature 25˜30° C.). To the residue were added water (150 mL) and 2N HCl until pH=3. A suspension was formed, filtered and washed with water. The product was collected and slurried from EA (60 mL) to give the product (10 g). The mother liquid was extracted with EA. The organic layer was concentrated. The residue was slurried with EA (20 mL) to give the product (6.5 g). The filtrate was concentrated and the residue was slurry with EtOH to give the product (1.1 g). The three-batch product was combined to yield the target compound (17.6 g, 89%) in total.

### Compound A63: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-phenylpropan-1-one

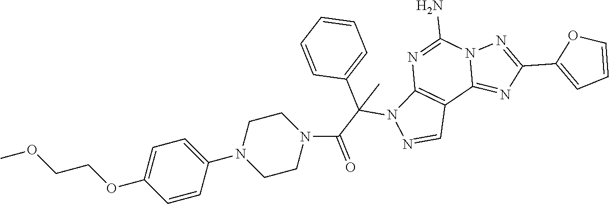 [SMILES: COCCOc1ccc(N2CCN(C(=O)C(C)(c3ccccc3)n3ncc4c3nc(N)n3nc(-c5ccco5)nc43)CC2)cc1]

A mixture of Intermediate-I (137 mg, 0.35 mmol), 1-(4-(2-methoxyethoxy)phenyl)piperazine (88 mg, 0.37 mmol), HATU (136.8 mg, 0.36 mmol) and DIPEA (91 mg, 0.7 mmol) in DMF (5 mL) was stirred overnight at RT. The reaction mixture was poured into H₂O (20 mL) and extracted with EA (20 mL×3). The combined organic layers were washed with brine, dried over Na₂SO₄, concentrated and purified by column chromatography (petroleum ether/EA=1:2) to give the target compound (79 mg, 37.17%). ¹H NMR (400 MHz, DMSO-d6) δ 8.31 (s, 1H), 8.08 (s, 2H), 7.95 (s, 1H), 7.35-7.19 (m, 6H), 6.76-6.70 (m, 5H), 3.96-3.94 (m, 2H), 3.70 (m, 2H), 3.59-3.57 (m, 2H), 3.27 (s, 3H), 2.95 (m, 6H), 2.33 (s, 3H) ppm. MS: M/e 608 (M+1)⁺.

### Compound A63a: (S)-2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-phenylpropan-1-one

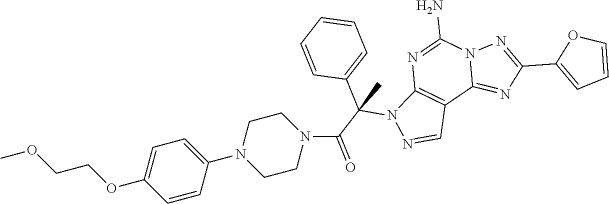 [SMILES: COCCOc1ccc(N2CCN(C(=O)[C@](C)(c3ccccc3)n3ncc4c3nc(N)n3nc(-c5ccco5)nc43)CC2)cc1]

A mixture of(S)-2-(5-amino-2-(furan-2-yl)-7H-pyrazolo[4,3-e][1,2,4]triazolo[1,5-c]pyrimidin-7-yl)-2-phenylpropanoic acid (30 mg, 0.077 mmol), 1-(4-(2-methoxyethoxy)phenyl)piperazine (22 mg, 0.09 mmol), HATU (35 mg, 0.09 mmol) and DIPEA (19 mg, 0.15 mmol) in DMF (5 mL) was stirred overnight at RT. The reaction mixture was poured into H₂O (60 mL) and extracted with EA (20 mL×3). The combined organic layers were washed with brine, dried over Na₂SO₄, concentrated in vacuo and purified by column chromatography (EA) to give the target compound (30 mg, 64.2%) as a white solid. ¹H NMR (400 MHz, DMSO-d6) δ 8.31 (s, 1H), 8.08 (s, 2H), 7.95 (s, 1H), 7.36-7.14 (m, 6H), 6.73 (m, 5H), 3.99-3.91 (m, 2H), 3.69 (s, 2H), 3.59-3.52 (m, 2H), 3.27 (s, 3H), 2.95 (m, 4H), 2.33 (s, 3H). MS: M/e 608 (M+1)⁺.

### Compound A63b: (R)-2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-phenylpropan-1-one

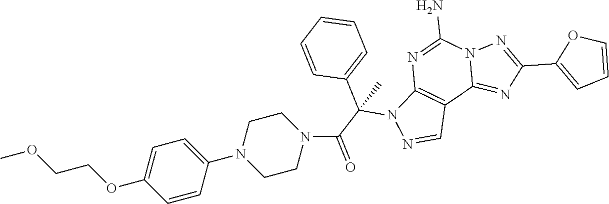 [SMILES: COCCOc1ccc(N2CCN(C(=O)[C@@](C)(c3ccccc3)n3ncc4c3nc(N)n3nc(-c5ccco5)nc43)CC2)cc1]

A mixture of (R)-2-(5-amino-2-(furan-2-yl)-7H-pyrazolo[4,3-e][1,2,4]triazolo[1,5-c]pyrimidin-7-yl)-2-phenylpropanoic acid (30 mg, 0.077 mmol), 1-(4-(2-methoxyethoxy)phenyl)piperazine (22 mg, 0.09 mmol), HATU (35 mg, 0.09 mmol) and DIPEA (19 mg, 0.15 mmol) in DMF (5 mL) was stirred overnight at RT. The reaction mixture was poured into H₂O (60 mL) and extracted with EA (20 mL×3). The combined organic layers were washed with brine, dried over Na₂SO₄, concentrated in vacuo and purified by column chromatography (EA) to give the target compound (31 mg, 64.2%) as a white solid. ¹H NMR (400 MHz, DMSO-d6) δ 8.31 (s, 1H), 8.08 (s, 2H), 7.95 (s, 1H), 7.38-7.11 (m, 6H), 6.73 (m, 5H), 4.00-3.89 (m, 2H), 3.69 (s, 2H), 3.62-3.57 (m, 2H), 3.27 (s, 3H), 2.95 (s, 4H), 2.33 (s, 3H). MS: M/e 608 (M+1)+.

### Compound A64: 2-(5-amino-2-(furan-2-yl)-7H-pyrazolo[4,3-e][1,2,4]triazolo[1,5-c]pyrimidin-7-yl)-1-(4-(2-fluoro-4-(2-methoxyethoxy)phenyl)piperazin-1-yl)-2-phenylpropan-1-one

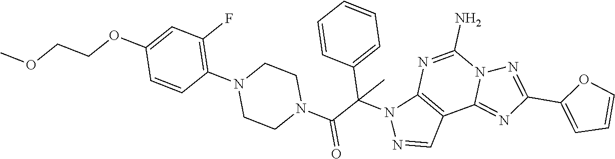 [SMILES: COCCOc1ccc(N2CCN(C(=O)C(C)(c3ccccc3)n3ncc4c3nc(N)n3nc(-c5ccco5)nc43)CC2)c(F)c1]

A mixture of 2-(5-amino-2-(furan-2-yl)-7H-pyrazolo[4,3-e][1,2,4]triazolo[1,5-c]pyrimidin-7-yl)-2-phenylpropanoic acid (50 mg, 0.128 mmol), 1-(2-fluoro-4-(2-methoxyethoxy)phenyl)piperazine hydrochloride (37.3 mg, 0.128 mmol), HATU (58.3 mg, 0.154 mmol) and DIPEA (33 mg, 0.256 mmol) in DMF (4 mL) was stirred overnight. The reaction mixture was poured into H₂O (10 mL) and extracted with EtOAc (10 mL×3). The combined organic layers were washed with brine, dried over Na₂SO₄, concentrated and purified by prep-TLC (petroleum ether/EtOAc=1:2) to give the target compound (23 mg, 28.75%). ¹H NMR (400 MHz, DMSO-d6) δ 8.30 (s, 1H), 8.08 (s, 2H), 7.95 (s, 1H), 7.43-7.11 (m, 6H), 6.85-6.66 (m, 3H), 6.64-6.55 (m, 1H), 4.02 (m, 2H), 3.85-3.55 (m, 5H), 3.26 (s, 3H), 3.15-2.73 (m, 5H), 2.34 (s, 3H) ppm. MS: M/e 626 (M+1)⁺.

### Compound A64a: (S)-2-(5-amino-2-(furan-2-yl)-7H-pyrazolo[4,3-e][1,2,4]triazolo[1,5-c]pyrimidin-7-yl)-1-(4-(2-fluoro-4-(2-methoxyethoxy)phenyl)piperazin-1-yl)-2-phenylpropan-1-one

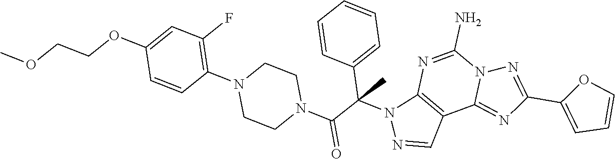 [SMILES: COCCOc1ccc(N2CCN(C(=O)[C@](C)(c3ccccc3)n3ncc4c3nc(N)n3nc(-c5ccco5)nc43)CC2)c(F)c1]

A mixture of (S)-2-(5-amino-2-(furan-2-yl)-7H-pyrazolo[4,3-e][1,2,4]triazolo[1,5-c]pyrimidin-7-yl)-2-phenylpropanoic acid (50 mg, 0.128 mmol), 1-(2-fluoro-4-(2-methoxyethoxy)phenyl)piperazine hydrochloride (37.3 mg, 0.128 mmol), HATU (58.3 mg, 0.154 mmol) and DIPEA (33 mg, 0.256 mmol) in DMF (4 mL) was stirred overnight. The reaction mixture was poured into H₂O (10 mL) and extracted with EtOAc (10 mL×3). The combined organic layers were washed with brine, dried over Na₂SO₄, concentrated and purified by prep-TLC (petroleum ether/EtOAc=1:2) to give the target compound (30 mg, 37.5%) as a white solid. ¹H NMR (400 MHz, DMSO-d6) δ 8.30 (s, 1H), 8.08 (s, 2H), 7.95 (s, 1H), 7.43-7.11 (m, 6H), 6.85-6.66 (m, 3H), 6.64-6.55 (m, 1H), 4.02 (m, 2H), 3.85-3.55 (m, 5H), 3.26 (s, 3H), 3.15-2.73 (m, 5H), 2.34 (s, 3H) ppm. MS: M/e 626 (M+1)⁺.

### Compound A64b: (R)-2-(5-amino-2-(furan-2-yl)-7H-pyrazolo[4,3-e][1,2,4]triazolo[1,5-c]pyrimidin-7-yl)-1-(4-(2-fluoro-4-(2-methoxyethoxy)phenyl)piperazin-1-yl)-2-phenylpropan-1-one

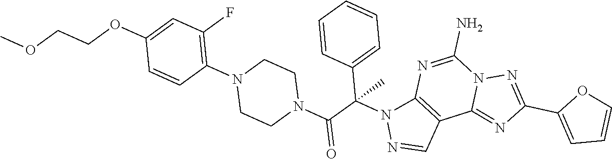 [SMILES: COCCOc1ccc(N2CCN(C(=O)[C@@](C)(c3ccccc3)n3ncc4c3nc(N)n3nc(-c5ccco5)nc43)CC2)c(F)c1]

A mixture of (R)-2-(5-amino-2-(furan-2-yl)-7H-pyrazolo[4,3-e][1,2,4]triazolo[1,5-c]pyrimidin-7-yl)-2-phenylpropanoic acid (50 mg, 0.128 mmol), 1-(2-fluoro-4-(2-methoxyethoxy)phenyl)piperazine hydrochloride (37.3 mg, 0.128 mmol), HATU (58.3 mg, 0.154 mmol) and DIPEA (33 mg, 0.256 mmol) in DMF (4 mL) was stirred overnight. The reaction mixture was poured into H₂O (10 mL) and extracted with EtOAc (10 mL×3). The combined organic layers were washed with brine, dried over Na₂SO₄, concentrated and purified by prep-TLC (petroleum ether/EtOAc=1:2) to give the target compound (30 mg, 37.5%) as a white solid. ¹H NMR (400 MHz, DMSO-d6) δ 8.30 (s, 1H), 8.08 (s, 2H), 7.95 (s, 1H), 7.43-7.11 (m, 6H), 6.85-6.66 (m, 3H), 6.64-6.55 (m, 1H), 4.02 (m, 2H), 3.85-3.55 (m, 5H), 3.26 (s, 3H), 3.15-2.73 (m, 5H), 2.34 (s, 3H) ppm. MS: M/e 626 (M+1)⁺.

### Compound A65: 2-(5-amino-2-(furan-2-yl)-7H-pyrazolo[4,3-e][1,2,4]triazolo[1,5-c]pyrimidin-7-yl)-1-(4-(2,5-difluoro-4-(2-methoxyethoxy)phenyl)piperazin-1-yl)-2-phenylpropan-1-one

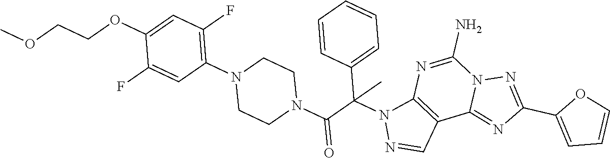 [SMILES: COCCOc1cc(F)c(N2CCN(C(=O)C(C)(c3ccccc3)n3ncc4c3nc(N)n3nc(-c5ccco5)nc43)CC2)cc1F]

A mixture of 2-(5-amino-2-(furan-2-yl)-7H-pyrazolo[4,3-e][1,2,4]triazolo[1,5-c]pyrimidin-7-yl)-2-phenylpropanoic acid (50 mg, 0.128 mmol), 1-(2,5-difluoro-4-(2-methoxyethoxy)phenyl)piperazine hydrochloride (39.5 mg, 0.128 mmol), HATU (58.3 mg, 0.154 mmol) and DIPEA (33 mg, 0.256 mmol) in DMF (4 mL) was stirred overnight. The reaction mixture was poured into H₂O (10 mL) and extracted with EtOAc (10 mL×3). The combined organic layers were washed with brine, dried over Na₂SO₄, concentrated and purified by prep-TLC (petroleum ether/EtOAc=1:2) to give the target compound (22 mg, 26.7%). ¹H NMR (400 MHz, DMSO-d6) δ 8.31 (s, 1H), 8.08 (s, 2H), 7.95 (s, 1H), 7.38-7.21 (m, 4H), 7.18 (d, J=7.2 Hz, 2H), 7.10-7.04 (m, 1H), 6.85-6.67 (m, 2H), 4.17-4.02 (m, 2H), 3.67 (m, 4H), 3.27 (s, 3H), 3.11-2.75 (m, 5H), 2.34 (s, 3H) ppm. MS: M/e 644 (M+1)⁺.

### Compound A65a: (S)-2-(5-amino-2-(furan-2-yl)-7H-pyrazolo[4,3-e][1,2,4]triazolo[1,5-c]pyrimidin-7-yl)-1-(4-(2,5-difluoro-4-(2-methoxyethoxy)phenyl)piperazin-1-yl)-2-phenylpropan-1-one

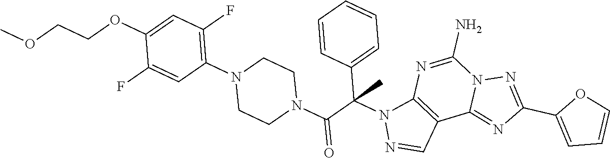 [SMILES: COCCOc1cc(F)c(N2CCN(C(=O)[C@](C)(c3ccccc3)n3ncc4c3nc(N)n3nc(-c5ccco5)nc43)CC2)cc1F]

A mixture of (S)-2-(5-amino-2-(furan-2-yl)-7H-pyrazolo[4,3-e][1,2,4]triazolo[1,5-c]pyrimidin-7-yl)-2-phenylpropanoic acid (70 mg, 0.18 mmol), 1-(2,5-difluoro-4-(2-methoxyethoxy)phenyl)piperazine hydrochloride (55.5 mg, 0.18 mmol), HATU (82 mg, 0.22 mmol) and DIPEA (46 mg, 0.36 mmol) in DMF (5 mL) was stirred overnight. The reaction mixture was poured into H₂O (20 mL) and extracted with EtOAc (15 mL×3). The combined organic layers were washed with brine, dried over Na₂SO₄, concentrated and purified by prep-TLC (petroleum ether/EtOAc=1:2) to give the target compound (50 mg, 43.2%) as a white solid. ¹H NMR (400 MHz, DMSO-d6) δ 8.31 (s, 1H), 8.08 (s, 2H), 7.95 (s, 1H), 7.38-7.21 (m, 4H), 7.18 (d, J=7.2 Hz, 2H), 7.10-7.04 (m, 1H), 6.85-6.67 (m, 2H), 4.17-4.02 (m, 2H), 3.67 (m, 4H), 3.27 (s, 3H), 3.11-2.75 (m, 5H), 2.34 (s, 3H) ppm. MS: M/e 644 (M+1)⁺.

### Compound A65b: (R)-2-(5-amino-2-(furan-2-yl)-7H-pyrazolo[4,3-e][1,2,4]triazolo[1,5-c]pyrimidin-7-yl)-1-(4-(2,5-difluoro-4-(2-methoxyethoxy)phenyl)piperazin-1-yl)-2-phenylpropan-1-one

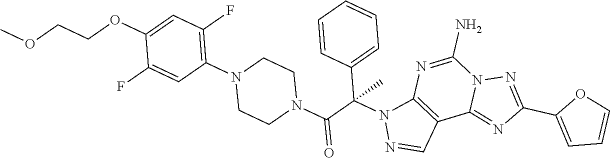 [SMILES: COCCOc1cc(F)c(N2CCN(C(=O)[C@@](C)(c3ccccc3)n3ncc4c3nc(N)n3nc(-c5ccco5)nc43)CC2)cc1F]

A mixture of (R)-2-(5-amino-2-(furan-2-yl)-7H-pyrazolo[4,3-e][1,2,4]triazolo[1,5-c]pyrimidin-7-yl)-2-phenylpropanoic acid (70 mg, 0.18 mmol), 1-(2,5-difluoro-4-(2-methoxyethoxy)phenyl)piperazine hydrochloride (55.5 mg, 0.18 mmol), HATU (82 mg, 0.22 mmol) and DIPEA (46 mg, 0.36 mmol) in DMF (5 mL) was stirred overnight. The reaction mixture was poured into H₂O (15 mL) and extracted with EtOAc (15 mL×3). The combined organic layers were washed with brine, dried over Na₂SO₄, concentrated and purified by prep-TLC (petroleum ether/EtOAc=1:2) to give the target compound (45 mg, 38.9%) as a white solid. ¹H NMR (400 MHz, DMSO-d6) δ 8.31 (s, 1H), 8.08 (s, 2H), 7.95 (s, 1H), 7.38-7.21 (m, 4H), 7.18 (d, J=7.2 Hz, 2H), 7.10-7.04 (m, 1H), 6.85-6.67 (m, 2H), 4.17-4.02 (m, 2H), 3.67 (m, 4H), 3.27 (s, 3H), 3.11-2.75 (m, 5H), 2.34 (s, 3H) ppm. MS: M/e 644 (M+1)⁺.

### Compound A66: 2-(5-amino-2-(furan-2-yl)-7H-pyrazolo[4,3-e][1,2,4]triazolo[1,5-c]pyrimidin-7-yl)-1-(4-(3-fluoro-4-(2-methoxyethoxy)phenyl)piperazin-1-yl)-2-phenylpropan-1-one

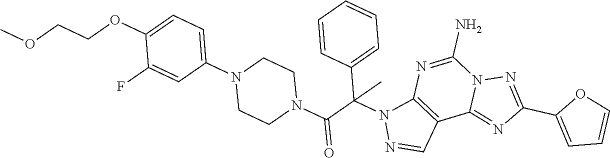 [SMILES: COCCOc1ccc(N2CCN(C(=O)C(C)(c3ccccc3)n3ncc4c3nc(N)n3nc(-c5ccco5)nc43)CC2)cc1F]

A mixture of 2-(5-amino-2-(furan-2-yl)-7H-pyrazolo[4,3-e][1,2,4]triazolo[1,5-c]pyrimidin-7-yl)-2-phenylpropanoic acid (50 mg, 0.128 mmol), 1-(3-fluoro-4-(2-methoxyethoxy)phenyl)piperazine hydrochloride (37.3 mg, 0.128 mmol), HATU (58.3 mg, 0.154 mmol) and DIPEA (33 mg, 0.256 mmol) in DMF (4 mL) was stirred overnight. The reaction mixture was poured into H₂O (10 mL) and extracted with EtOAc (10 mL×3). The combined organic layers were washed with brine, dried over Na₂SO₄, concentrated and purified by prep-TLC (petroleum ether/EtOAc=1:2) to give the target compound (27 mg, 33.75%). ¹H NMR (400 MHz, DMSO-d6) δ 8.31 (s, 1H), 8.07 (s, 2H), 7.95 (s, 1H), 7.41-7.13 (m, 6H), 6.93 (t, J=9.6 Hz, 1H), 6.78-6.65 (m, 2H), 6.50 (d, J=8.8 Hz, 1H), 4.08-3.94 (m, 2H), 3.82-3.55 (m, 5H), 3.14-2.84 (m, 5H), 2.33 (s, 3H) ppm. MS: M/e 626 (M+1)⁺.

### Compound A66a: (S)-2-(5-amino-2-(furan-2-yl)-7H-pyrazolo[4,3-e][1,2,4]triazolo[1,5-c]pyrimidin-7-yl)-1-(4-(3-fluoro-4-(2-methoxyethoxy)phenyl)piperazin-1-yl)-2-phenylpropan-1-one

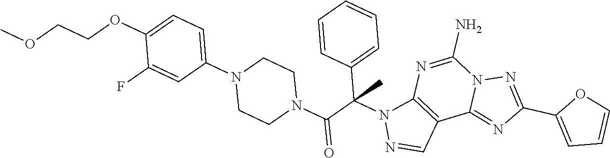 [SMILES: COCCOc1ccc(N2CCN(C(=O)[C@](C)(c3ccccc3)n3ncc4c3nc(N)n3nc(-c5ccco5)nc43)CC2)cc1F]

A mixture of (S)-2-(5-amino-2-(furan-2-yl)-7H-pyrazolo[4,3-e][1,2,4]triazolo[1,5-c]pyrimidin-7-yl)-2-phenylpropanoic acid (70 mg, 0.18 mmol), 1-(3-fluoro-4-(2-methoxyethoxy)phenyl)piperazine hydrochloride (53 mg, 0.18 mmol), HATU (82 mg, 0.22 mmol) and DIPEA (46 mg, 0.36 mmol) in DMF (5 mL) was stirred overnight. The reaction mixture was poured into H₂O (10 mL) and extracted with EtOAc (15 mL×3). The combined organic layers were washed with brine, dried over Na₂SO₄, concentrated and purified by prep-TLC (petroleum ether/EtOAc=1:2) to give the target compound (35 mg, 31.1%) as a white solid. ¹H NMR (400 MHz, DMSO-d6) δ 8.31 (s, 1H), 8.07 (s, 2H), 7.95 (s, 1H), 7.41-7.13 (m, 6H), 6.93 (t, J=9.6 Hz, 1H), 6.78-6.65 (m, 2H), 6.50 (d, J=8.8 Hz, 1H), 4.08-3.94 (m, 2H), 3.82-3.55 (m, 5H), 3.14-2.84 (m, 5H), 2.33 (s, 3H) ppm. MS: M/e 626 (M+1)⁺.

### Compound A66b: (R)-2-(5-amino-2-(furan-2-yl)-7H-pyrazolo[4,3-e][1,2,4]triazolo[1,5-c]pyrimidin-7-yl)-1-(4-(3-fluoro-4-(2-methoxyethoxy)phenyl)piperazin-1-yl)-2-phenylpropan-1-one

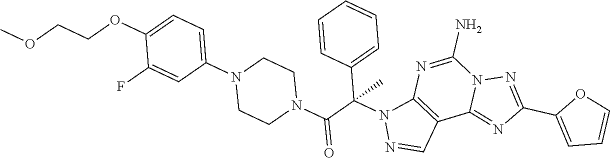 [SMILES: COCCOc1ccc(N2CCN(C(=O)[C@@](C)(c3ccccc3)n3ncc4c3nc(N)n3nc(-c5ccco5)nc43)CC2)cc1F]

A mixture of (R)-2-(5-amino-2-(furan-2-yl)-7H-pyrazolo[4,3-e][1,2,4]triazolo[1,5-c]pyrimidin-7-yl)-2-phenylpropanoic acid (70 mg, 0.18 mmol), 1-(3-fluoro-4-(2-methoxyethoxy)phenyl)piperazine hydrochloride (53 mg, 0.18 mmol), HATU (82 mg, 0.22 mmol) and DIPEA (46 mg, 0.36 mmol) in DMF (4 mL) was stirred overnight. The reaction mixture was poured into H₂O (15 mL) and extracted with EtOAc (15 mL×3). The combined organic layers were washed with brine, dried over Na₂SO₄, concentrated and purified by prep-TLC (petroleum ether/EtOAc=1:2) to give the target compound (38 mg, 33.8%) as a white solid. ¹H NMR (400 MHz, DMSO-d6) δ 8.31 (s, 1H), 8.07 (s, 2H), 7.95 (s, 1H), 7.41-7.13 (m, 6H), 6.93 (t, J=9.6 Hz, 1H), 6.78-6.65 (m, 2H), 6.50 (d, J=8.8 Hz, 1H), 4.08-3.94 (m, 2H), 3.82-3.55 (m, 5H), 3.14-2.84 (m, 5H), 2.33 (s, 3H) ppm. MS: M/e 626 (M+1)⁺.

### Compound A67: 2-(5-amino-2-(furan-2-yl)-7H-pyrazolo[4,3-e][1,2,4]triazolo[1,5-c]pyrimidin-7-yl)-1-(4-(2,3-difluoro-4-(2-methoxyethoxy)phenyl)piperazin-1-yl)-2-phenylpropan-1-one

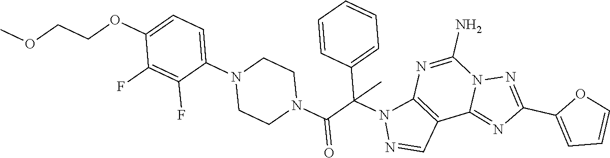 [SMILES: COCCOc1ccc(N2CCN(C(=O)C(C)(c3ccccc3)n3ncc4c3nc(N)n3nc(-c5ccco5)nc43)CC2)c(F)c1F]

A mixture of 2-(5-amino-2-(furan-2-yl)-7H-pyrazolo[4,3-e][1,2,4]triazolo[1,5-c]pyrimidin-7-yl)-2-phenylpropanoic acid (50 mg, 0.128 mmol), 1-(2,3-difluoro-4-(2-methoxyethoxy)phenyl)piperazine hydrochloride (39.5 mg, 0.128 mmol), HATU (58.3 mg, 0.154 mmol) and DIPEA (33 mg, 0.256 mmol) in DMF (4 mL) was stirred overnight. The reaction mixture was poured into H₂O (10 mL) and extracted with EtOAc (10 mL×3). The combined organic layers were washed with brine, dried over Na₂SO₄, concentrated and purified by prep-TLC (petroleum ether/EtOAc=1:2) to give the target compound (33 mg, 41%). ¹H NMR (400 MHz, DMSO-d6) δ 8.31 (s, 1H), 8.08 (s, 2H), 7.96 (s, 1H), 7.42-7.13 (m, 6H), 6.83 (t, J=8.8 Hz, 1H), 6.77-6.71 (m, 1H), 6.56 (t, J=8.8 Hz, 1H), 4.12-4.05 (m, 2H), 3.90-3.57 (m, 5H), 3.18-2.78 (m, 5H), 2.34 (s, 3H) ppm. MS: M/e 644 (M+1)⁺.

### Compound A68: 2-(5-amino-2-(furan-2-yl)-7H-pyrazolo[4,3-e][1,2,4]triazolo[1,5-c]pyrimidin-7-yl)-1-(4-(2,4-difluorophenyl)piperazin-1-yl)-2-phenylpropan-1-one

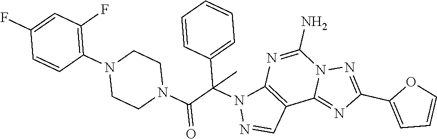 [SMILES: CC(C(=O)N1CCN(c2ccc(F)cc2F)CC1)(c1ccccc1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)-2-phenylpropanoic acid (50 mg, 0.13 mmol), 1-(2,4-difluorophenyl)piperazine (25 mg, 0.13 mmol), HATU (59 mg, 0.16 mmol) and DIEA (33 mg, 0.26 mmol) in DMF (5 mL) was stirred at rt for 2 hrs. The solution was added with water (10 mL), extracted with ethyl acetate (10 mL) and washed with brine (10 mL). The organic layer was dried, concentrated and purified by column chromatography (PE:EA=2:1) and preparative TLC (DCM:MeOH=20:1) to get the desired product (20 mg, 28%). ¹H NMR (400 MHz, DMSO-d6) δ 8.31 (s, 1H), 8.08 (br.s, 2H), 7.95 (d, J=4.0 Hz, 1H), 7.30-7.11 (m, 7H), 6.92-6.86 (m, 2H), 6.74 (d, J=4.0 Hz, 1H), 3.71 (br.s, 2H), 3.45-3.40 (m, 2H), 3.16-2.90 (m, 4H), 2.35 (s, 3H) ppm. MS: M/e 570 (M+1)⁺

### Compound A69: 2-(5-amino-2-(furan-2-yl)-7H-pyrazolo[4,3-e][1,2,4]triazolo[1,5-c]pyrimidin-7-yl)-1-(4-(2-fluorophenyl)piperazin-1-yl)-2-phenylpropan-1-one

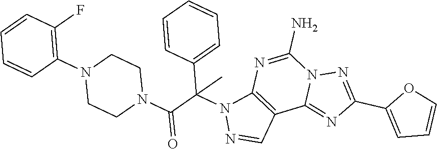 [SMILES: CC(C(=O)N1CCN(c2ccccc2F)CC1)(c1ccccc1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)-2-phenylpropanoic acid (50 mg, 0.13 mmol), 1-(2-fluorophenyl)piperazine (24 mg, 0.13 mmol), HATU (60 mg, 0.16 mmol) and DIEA (34 mg, 0.26 mmol) in DMF (5 mL) was stirred at rt for 2 hrs. The solution was added with water (10 mL), extracted with ethyl acetate (10 mL) and washed with brine (10 mL). The organic layer was dried, concentrated and purified by preparative TLC (DCM:MeOH=30:1) to get the desired product (20 mg, 29%). ¹H NMR (400 MHz, DMSO-d6) δ 8.31 (s, 1H), 8.08 (br.s, 2H), 7.95 (d, J=4.0 Hz, 1H), 7.31-7.18 (m, 6H), 7.09-6.74 (m, 5H), 3.73-3.39 (m, 4H), 3.16-2.90 (m, 4H), 2.35 (s, 3H) ppm. MS: M/e 552 (M+1)⁺

### Compound A70: 2-(5-amino-2-(furan-2-yl)-7H-pyrazolo[4,3-e][1,2,4]triazolo[1,5-c]pyrimidin-7-yl)-1-(4-(4-fluorophenyl)piperazin-1-yl)-2-phenylpropan-1-one

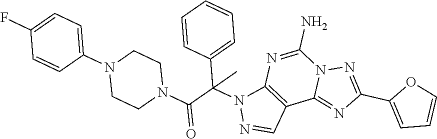 [SMILES: CC(C(=O)N1CCN(c2ccc(F)cc2)CC1)(c1ccccc1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)-2-phenylpropanoic acid (50 mg, 0.13 mmol), 1-(4-fluorophenyl)piperazine hydrochloride (28 mg, 0.13 mmol), HATU (60 mg, 0.16 mmol) and DIEA (34 mg, 0.26 mmol) in DMF (5 mL) was stirred at rt for 2 hrs. The solution was added with water (10 mL), extracted with ethyl acetate (10 mL) and washed with brine (10 mL). The organic layer was dried, concentrated and purified by preparative TLC (DCM:MeOH=30:1) and preparative HPLC to get the desired product (16 mg, 23%). ¹H NMR (400 MHz, DMSO-d6) δ 8.31 (s, 1H), 8.08 (br.s, 2H), 7.95 (d, J=4.0 Hz, 1H), 7.33-7.18 (m, 6H), 6.96-6.94 (m, 2H), 6.82-6.73 (m, 3H), 3.72-3.36 (m, 4H), 3.17-2.91 (m, 4H), 2.35 (s, 3H) ppm. MS: M/e 552 (M+1)⁺

### Compound A70a: (S)-2-(5-amino-2-(furan-2-yl)-7H-pyrazolo[4,3-e][1,2,4]triazolo[1,5-c]pyrimidin-7-yl)-1-(4-(4-fluorophenyl)piperazin-1-yl)-2-phenylpropan-1-one

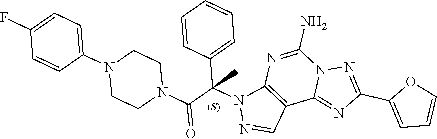

A mixture of(S)-2-(5-amino-2-(furan-2-yl)-7H-pyrazolo[4,3-e][1,2,4]triazolo[1,5-c]pyrimidin-7-yl)-2-phenylpropanoic acid (50 mg, 0.13 mmol), 1-(4-fluorophenyl)piperazine hydrochloride (33 mg, 0.15 mmol), HATU (57 mg, 0.15 mmol) and DIEA (50 mg, 0.39 mmol) in DMF (10 mL) was stirred at rt for 2 hrs. The solution was added with water (10 mL), extracted with ethyl acetate (10 mL) and washed with brine (10 mL). The organic layer was dried, concentrated and purified by column chromatography (PE:EA=1:1 to EA) to get the desired product as a white solid (32 mg, 46%). ¹H NMR (400 MHz, DMSO-d6) δ 8.31 (s, 1H), 8.08 (br.s, 2H), 7.95 (s, 1H), 7.34-7.18 (m, 6H), 6.97 (t, J=8.0 Hz, 2H), 6.82-6.73 (m, 3H), 3.75-3.45 (m, 4H), 3.20-2.80 (m, 4H), 2.34 (s, 3H) ppm. MS: M/e 552 (M+1)⁺

### Compound A70b: (R)-2-(5-amino-2-(furan-2-yl)-7H-pyrazolo[4,3-e][1,2,4]triazolo[1,5-c]pyrimidin-7-yl)-1-(4-(4-fluorophenyl)piperazin-1-yl)-2-phenylpropan-1-one

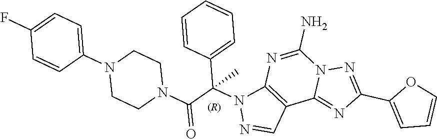

A mixture of (R)-2-(5-amino-2-(furan-2-yl)-7H-pyrazolo[4,3-e][1,2,4]triazolo[1,5-c]pyrimidin-7-yl)-2-phenylpropanoic acid (50 mg, 0.13 mmol), 1-(4-fluorophenyl)piperazine hydrochloride (33 mg, 0.15 mmol), HATU (57 mg, 0.15 mmol) and DIEA (50 mg, 0.39 mmol) in DMF (10 mL) was stirred at rt for 2 hrs. The solution was added with water (10 mL), extracted with ethyl acetate (10 mL) and washed with brine (10 mL). The organic layer was dried, concentrated and purified by column chromatography (PE:EA=1:1 to EA) to get the desired product as a white solid (35 mg, 49%). ¹H NMR (400 MHz, DMSO-d6) δ 8.31 (s, 1H), 8.07 (br.s, 2H), 7.95 (s, 1H), 7.31-7.18 (m, 6H), 6.96-6.93 (m, 2H), 6.82-6.74 (m, 3H), 3.75-3.45 (m, 4H), 3.20-2.80 (m, 4H), 2.34 (s, 3H) ppm. MS: M/e 552 (M+1)⁺

### Compound A71: 2-(5-amino-2-(furan-2-yl)-7H-pyrazolo[4,3-e][1,2,4]triazolo[1,5-c]pyrimidin-7-yl)-1-(4-(4-(2-(dimethylamino)ethoxy)phenyl)piperazin-1-yl)-2-phenylpropan-1-one

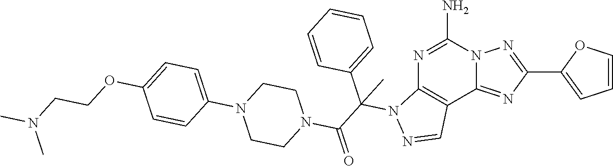 [SMILES: CN(C)CCOc1ccc(N2CCN(C(=O)C(C)(c3ccccc3)n3ncc4c3nc(N)n3nc(-c5ccco5)nc43)CC2)cc1]

A mixture of N,N-dimethyl-2-(4-(piperazin-1-yl)phenoxy)ethan-1-amine hydrochloride (113 mg, 0.45 mmol), 2-(5-amino-2-(furan-2-yl)-7H-pyrazolo[4,3-e][1,2,4]triazolo[1,5-c]pyrimidin-7-yl)-2-phenylpropanoic acid (177 mg, 0.45 mmol), HATU (172 mg, 0.45 mmol) and Et₃N (92 mg, 0.9 mmol) in DMF (10 mL) was stirred at RT overnight. The reaction mixture was poured into water (20 mL) and the solid was precipitated from the system. The solid was filtered and purified by prep-HPLC to afford the title compound (120 mg, yield: 42.7%). ¹H NMR (400 MHz, DMSO-d6) δ 8.32 (s, 1H), 8.09 (br.s, 2H), 7.95 (s, 1H), 7.37-7.27 (m, 3H), 7.24 (d, J=4 Hz, 1H), 7.21-7.13 (m, 2H), 6.86-6.72 (m, 5H), 4.19-4.11 (m, 4H), 3.81-3.62 (m, 2H), 3.48-3.39 (m, 2H), 3.09-2.85 (m, 4H), 2.85-2.73 (m, 6H), 2.35-2.28 (m, 3H) ppm. MS: M/e 621 (M+1)⁺.

Compound A71 was separated into two enantiomeric stereoisomers, Compound 71a (earlier peak), and Compound 71b (later peak) by chiral prep-HPLC. The chiral separation conditions are shown below.

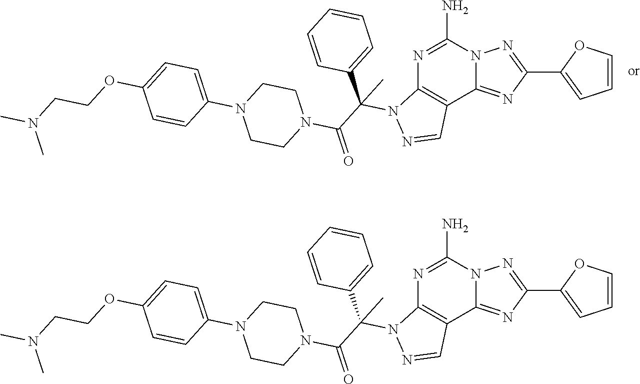 [SMILES: CN(C)CCOc1ccc(N2CCN(C(=O)[C@@](C)(c3ccccc3)n3ncc4c3nc(N)n3nc(-c5ccco5)nc43)CC2)cc1 ; CN(C)CCOc1ccc(N2CCN(C(=O)[C@](C)(c3ccccc3)n3ncc4c3nc(N)n3nc(-c5ccco5)nc43)CC2)cc1]

| Column | CHIRAL ART Cellulose-SB |
|---|---|
| Column size | 2 cm × 25 cm, 5 um |
| Injection | 0.8 ml |
| Mobile phase | (Hex:DCM = 5:1):EtOH = 50:50 |
| Flow rate | 20 ml/min |
| Wavelength | UV 220 nm |
| Temperature | 25° C. |
| Sample solution | 20.6 mg/ml in EtOH:DCM = 1:1 |
| Prep-HPLC equipment | Prep-HPLC-Gilson |

### Compound A72: 2-(5-amino-2-(furan-2-yl)-7H-pyrazolo[4,3-e][1,2,4]triazolo[1,5-c]pyrimidin-7-yl)-1-((S)-4-(4-(2-methoxyethoxy)phenyl)-2-methylpiperazin-1-yl)-2-phenylpropan-1-one

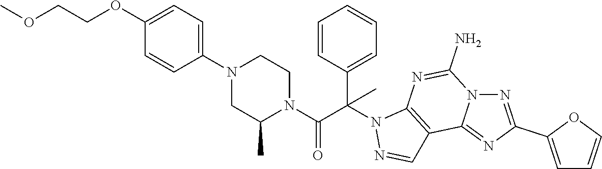 [SMILES: COCCOc1ccc(N2CCN(C(=O)C(C)(c3ccccc3)n3ncc4c3nc(N)n3nc(-c5ccco5)nc43)[C@@H](C)C2)cc1]

To a mixture of (S)-1-(4-(2-methoxyethoxy)phenyl)-3-methylpiperazine 2,2,2-trifluoroacetate (220 mg, 0.60 mmol), 2-(5-amino-2-(furan-2-yl)-7H-pyrazolo[4,3-e][1,2,4]triazolo[1,5-c]pyrimidin-7-yl)-2-phenylpropanoic acid (105 mg, 0.27 mmol), DIEA (200 mg, 1.5 mmol) in DMF (4 mL) was added HATU (130 mg, 0.34 mmol) at rt and the mixture was stirred at rt for 16 hrs. The mixture was diluted with 20 mL of EA and washed with NaHCO₃ (aq. 10 mL×2), brine (10 mL×3), dried over Na₂SO₄ and concentrated, purified by prep-TLC and prep-HPLC to give the title product (12 mg, yield: 6%). ¹H NMR (400 MHz, DMSO-d6) δ 8.36-8.23 (m, 1H), 8.19-7.99 (m, 2H), 7.95 (s, 1H), 7.46-7.09 (m, 6H), 6.83-6.62 (m, 5H), 4.00-3.91 (m, 2H), 3.61-3.49 (m, 4H), 3.27 (s, 3H), 3.08-2.63 (m, 4H), 2.35-2.13 (m, 4H), 1.34-1.08 (m, 3H). MS: M/e 622 (M+1)⁺.

### Compound A73: 2-(5-amino-2-(furan-2-yl)-7H-pyrazolo[4,3-e][1,2,4]triazolo[1,5-c]pyrimidin-7-yl)-1-((R)-4-(4-(2-methoxyethoxy)phenyl)-3-methylpiperazin-1-yl)-2-phenylpropan-1-one

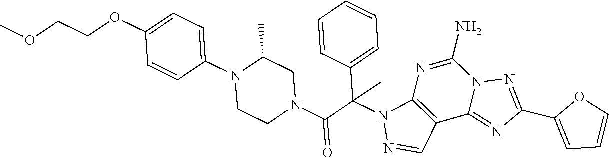 [SMILES: COCCOc1ccc(N2CCN(C(=O)C(C)(c3ccccc3)n3ncc4c3nc(N)n3nc(-c5ccco5)nc43)C[C@H]2C)cc1]

To a mixture of (R)-1-(4-(2-methoxyethoxy)phenyl)-2-methylpiperazine hydrochloride (100 mg, 0.35 mmol), 2-(5-amino-2-(furan-2-yl)-7H-pyrazolo[4,3-e][1,2,4]triazolo[1,5-c]pyrimidin-7-yl)-2-phenylpropanoic acid (88 mg, 0.23 mmol), DIEA (150 mg, 1.16 mmol) in DMF (3 mL) was added HATU (115 mg, 0.30 mmol) at rt and the mixture was stirred at rt for 16 hrs. The mixture was diluted with 20 mL of EA and washed with NaHCO₃ (aq. 10 mL×2), brine (10 mL×3), dried over Na₂SO₄ and concentrated, purified by prep-TLC and prep-HPLC to give the title product (32 mg, yield: 19%). ¹H NMR (400 MHz, DMSO-d6) δ 8.34 (d, J=18.0 Hz, 1H), 8.26-7.99 (m, 2H), 7.96 (s, 1H), 7.39-7.14 (m, 6H), 7.01-6.67 (m, 5H), 4.08-3.90 (m, 2H), 3.88-3.51 (m, 4H), 3.27 (d, J=1.6 Hz, 3H), 3.22-2.58 (m, 4H), 2.42-2.13 (m, 4H), 0.96-0.40 (m, 3H). MS: M/e 622 (M+1)⁺.

### Compound A74: 2-(5-amino-2-(furan-2-yl)-7H-pyrazolo[4,3-e][1,2,4]triazolo[1,5-c]pyrimidin-7-yl)-1-((S)-4-(4-(2-methoxyethoxy)phenyl)-3-methylpiperazin-1-yl)-2-phenylpropan-1-one

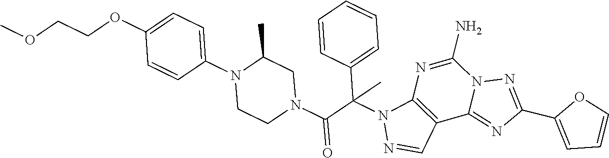 [SMILES: COCCOc1ccc(N2CCN(C(=O)C(C)(c3ccccc3)n3ncc4c3nc(N)n3nc(-c5ccco5)nc43)C[C@@H]2C)cc1]

To a mixture of (S)-1-(4-(2-methoxyethoxy)phenyl)-2-methylpiperazine hydrochloride (100 mg, 0.35 mmol), 2-(5-amino-2-(furan-2-yl)-7H-pyrazolo[4,3-e][1,2,4]triazolo[1,5-c]pyrimidin-7-yl)-2-phenylpropanoic acid (88 mg, 0.23 mmol), DIEA (150 mg, 1.16 mmol) in DMF (3 mL) was added HATU (115 mg, 0.30 mmol) at rt and the mixture was stirred at rt for 16 hrs. The mixture was diluted with 20 mL of EA and washed with NaHCO₃ (aq. 10 mL×2), brine (10 mL×3), dried over Na₂SO₄ and concentrated, purified by prep-TLC and prep-HPLC to give the title product (28 mg, yield: 17%). ¹H NMR (400 MHz, DMSO-d6) δ 8.34 (d, J=18.0 Hz, 1H), 8.26-8.01 (m, 2H), 7.96 (s, 1H), 7.40-7.06 (m, 6H), 6.96-6.52 (m, 5H), 4.08-3.92 (m, 2H), 3.92-3.45 (m, 4H), 3.27 (d, J=2.0 Hz, 3H), 3.23-2.52 (m, 4H), 2.44-2.11 (m, 4H), 0.95-0.27 (m, 3H). MS: M/e 622 (M+1)⁺.

### Compound A75: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(m-tolyl)propan-1-one

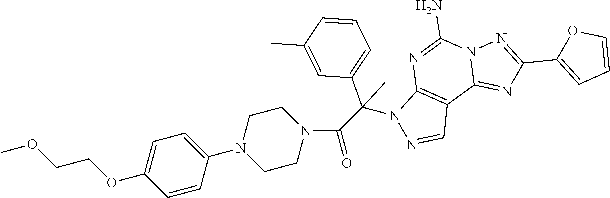 [SMILES: COCCOc1ccc(N2CCN(C(=O)C(C)(c3cccc(C)c3)n3ncc4c3nc(N)n3nc(-c5ccco5)nc43)CC2)cc1]

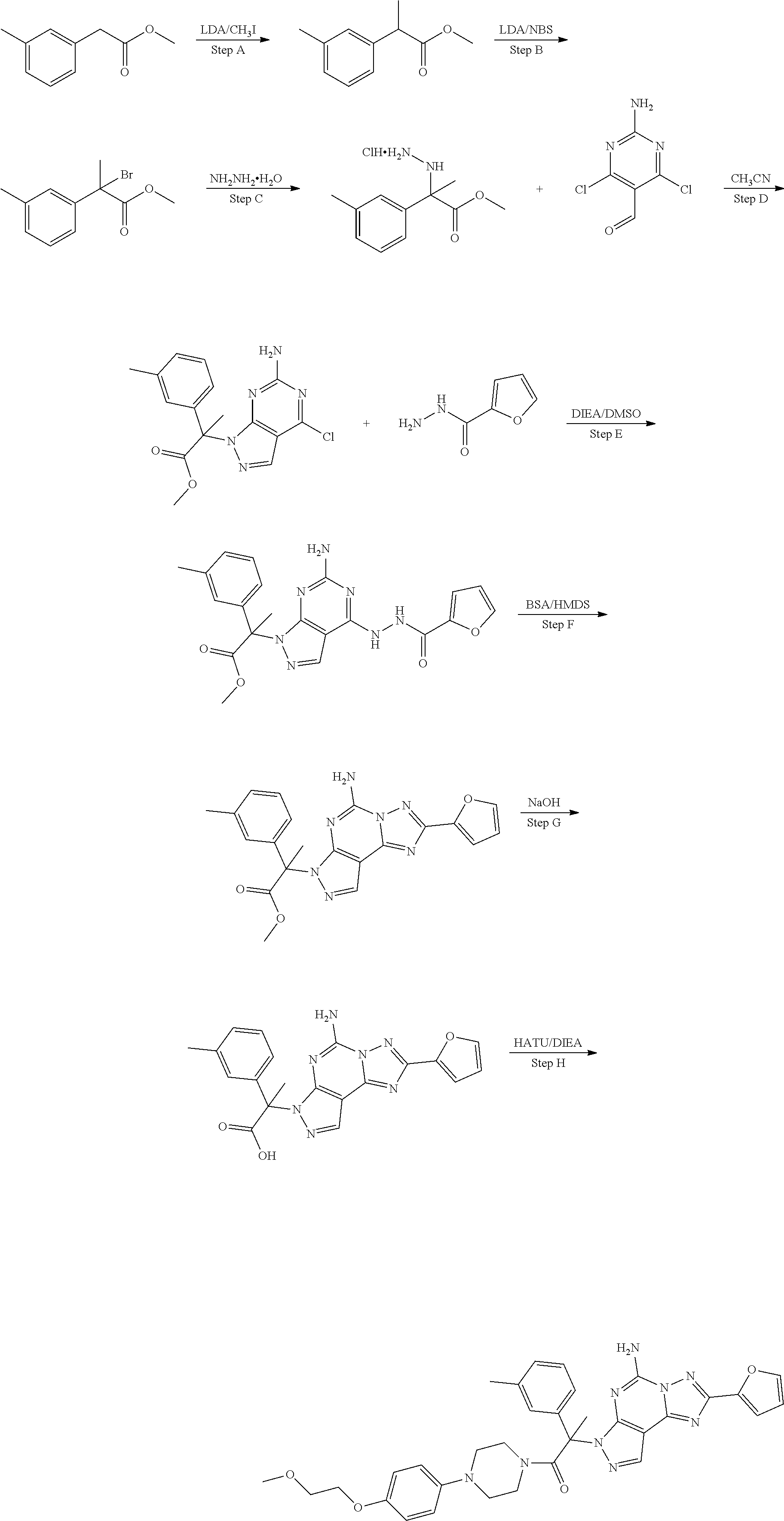

### Step A: methyl 2-(m-tolyl)propanoate

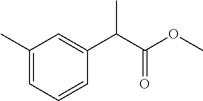

To a cooled solution of LDA (2M in THF, 10 mL, 20 mmol) in THF (60 mL) at −78° C. under N₂ atmosphere was added a solution of methyl 2-(m-tolyl)acetate (3 g, 18 mmol) in THF (5 mL) dropwise. After addition, the solution was stirred for further 20 mins before CH₃I (7.8 g, 55 mmol) was added dropwise. The mixture was allowed to warm to rt for 2 hrs. TLC showed the reaction was complete. The reaction mixture was quenched with water (5 mL), extracted with ethyl acetate (30 mL) and washed with brine (20 mL). The organic layer was dried, concentrated and purified by column chromatography (PE:EA=50:1) to get the product as an colorless oil (2.6 g, 81%). ¹H NMR (400 MHz, CDCl₃) δ 7.26-7.20 (m, 1H), 7.11-7.07 (m, 3H), 3.70-3.68 (m, 1H), 3.66 (s, 3H), 2.35 (s, 3H), 1.49 (d, J=8.0 Hz, 3H) ppm.

### Step B: methyl 2-bromo-2-(m-tolyl)propanoate

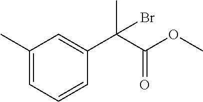

To a cooled solution of LDA (2M in THF, 3.1 mL, 6.2 mmol) in THF (30 mL) at −78° C. under N₂ atmosphere was added with a solution of methyl 2-(m-tolyl)propanoate (1 g, 5.6 mmol) in THF (2 mL) dropwise. The solution was stirred for further 30 mins before the dropwise addition of TMSCl (671 mg, 6.2 mmol). The mixture was allowed to warm to rt for 2 hrs before being cooled to −78° C. NBS (2 g, 11.2 mmol) was added in portions and the solution was stirred at −78° C. for 1 hr. The reaction mixture was allowed to reach to rt over 1 hr, then quenched with saturated NaHCO₃ solution, and extracted with ethyl acetate (20 mL). The organic layer was dried, concentrated and purified by column chromatography (PE:EA=50:1) to get the product (1 g, 70%). ¹H NMR (400 MHz, CDCl₃) δ 7.35-7.19 (m, 4H), 3.79 (s, 3H), 2.37 (s, 3H), 2.29 (s, 3H) ppm.

### Step C: methyl 2-hydrazinyl-2-(m-tolyl)propanoate

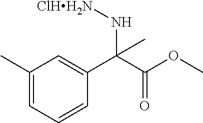

NH₂NH₂.H₂O (625 mg, 10 mmol) was added to a solution of methyl 2-bromo-2-(m-tolyl)propanoate (500 mg, 2 mmol) in CH₃CN (5 mL). The reaction mixture was heated at 60° C. for 1 hr. TLC showed the reaction was complete. The solution was concentrated, added with water (10 mL) and extracted with ethyl acetate (10 mL). The organic phase was concentrated, added with HCl/EA (2 M, 2 mL) and evaporated to get the product as HCl salt (380 mg, 80%). MS: M/e 209 (M+1)⁺

### Step D: methyl 2-(6-amino-4-chloro-1H-pyrazolo[3,4-d]pyrimidin-1-yl)-2-(m-tolyl)propanoate

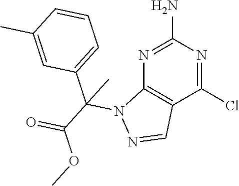 [SMILES: COC(=O)C(C)(c1cccc(C)c1)n1ncc2c(Cl)nc(N)nc21]

A mixture of methyl 2-hydrazinyl-2-(m-tolyl)propanoate hydrochloride (380 mg, 1.5 mmol) and 2-amino-4,6-dichloropyrimidine-5-carbaldehyde (300 mg, 1.5 mmol) in CH₃CN (10 mL) was stirred at rt overnight, and then heated at 50° C. for 3 hrs. The reaction mixture was evaporated, added with water (10 mL) and extracted with ethyl acetate (10 mL). The organic phase was dried, concentrated and further purified by column chromatography (PE:EA=6:1) to get the desired product as a white solid (300 mg, 56%). MS: M/e 346 (M+1)⁺

### Step E: methyl 2-(6-amino-4-(2-(furan-2-carbonyl)hydrazinyl)-1H-pyrazolo[3,4-d]pyrimidin-1-yl)-2-(m-tolyl)propanoate

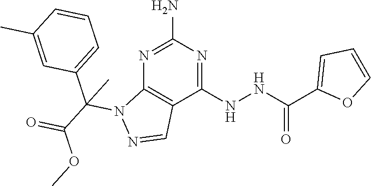 [SMILES: COC(=O)C(C)(c1cccc(C)c1)n1ncc2c(NNC(=O)c3ccco3)nc(N)nc21]

A mixture of methyl 2-(6-amino-4-chloro-1H-pyrazolo[3,4-d]pyrimidin-1-yl)-2-(m-tolyl)propanoate (300 mg, 0.87 mmol), furan-2-carbohydrazide (109 mg, 0.87 mmol) and DIEA (225 mg, 1.74 mmol) in DMSO (10 mL) was heated at 120° C. overnight. The solvent was evaporated under oil pump. The residue was added with water (10 mL), slurried and filtered. The cake was washed with water, dried to get the crude product, which was used in the next step directly (260 mg, 68%). MS: M/e 436 (M+1)⁺

### Step F: methyl 2-(5-amino-2-(furan-2-yl)-7H-pyrazolo[4,3-e][1,2,4]triazolo[1,5-c]pyrimidin-7-yl)-2-(m-tolyl)propanoate

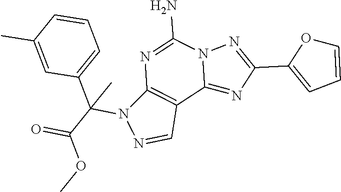 [SMILES: COC(=O)C(C)(c1cccc(C)c1)n1ncc2c1nc(N)n1nc(-c3ccco3)nc21]

A solution of methyl 2-(6-amino-4-(2-(furan-2-carbonyl)hydrazinyl)-1H-pyrazolo[3,4-d]pyrimidin-1-yl)-2-(m-tolyl)propanoate (260 mg, 0.60 mmol) in BSA (2 mL) and HMDS (2 mL) was heated at 120° C. for 3 hrs. The solvent was evaporated under oil pump. The residue was added with water (5 mL), extracted with ethyl acetate (10 mL) and washed with brine (10 mL). The organic layer was dried, concentrated and purified by column chromatography (PE:EA=5:1) to get the desired product as a white solid (110 mg, 44%). MS: M/e 418 (M+1)⁺

### Step G: 2-(5-amino-2-(furan-2-yl)-7H-pyrazolo[4,3-e][1,2,4]triazolo[1,5-c]pyrimidin-7-yl)-2-(m-tolyl)propanoic Acid

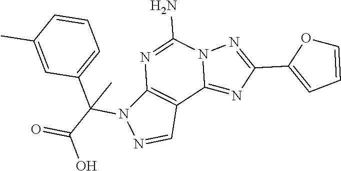 [SMILES: Cc1cccc(C(C)(C(=O)O)n2ncc3c2nc(N)n2nc(-c4ccco4)nc32)c1]

NaOH solution (50 mg, 1.25 mmol, in 1 mL of water) was added to a solution of methyl 2-(5-amino-2-(furan-2-yl)-7H-pyrazolo[4,3-e][1,2,4]triazolo[1,5-c]pyrimidin-7-yl)-2-(m-tolyl)propanoate (110 mg, 0.26 mmol) in methanol (5 mL). The solution was stirred at rt overnight. The solvent was evaporated. The residue was added with water (5 mL), acidified with 2 M HCl to pH=2˜3. The precipitated solid was filtered and dried to get the product as a white solid (40 mg, 38%). MS: M/e 404 (M+1)⁺

### Step H: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(m-tolyl)propan-1-one

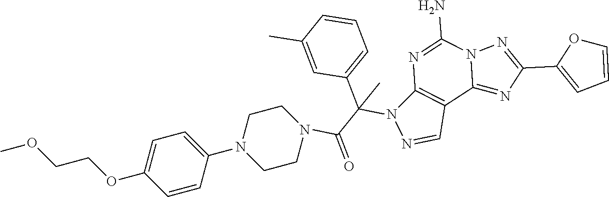 [SMILES: COCCOc1ccc(N2CCN(C(=O)C(C)(c3cccc(C)c3)n3ncc4c3nc(N)n3nc(-c5ccco5)nc43)CC2)cc1]

A mixture of 2-(5-amino-2-(furan-2-yl)-7H-pyrazolo[4,3-e][1,2,4]triazolo[1,5-c]pyrimidin-7-yl)-2-(m-tolyl)propanoic acid (25 mg, 0.06 mmol), 1-(4-(2-methoxyethoxy)phenyl)piperazine (18 mg, 0.07 mmol), HATU (27 mg, 0.07 mmol) and DIEA (16 mg, 0.12 mmol) in DMF (5 mL) was stirred at rt for 2 hrs. The solution was added with water (10 mL), extracted with ethyl acetate (10 mL) and washed with brine (10 mL). The organic layer was dried, concentrated and purified by preparative HPLC to get the desired product (12 mg, 32%). ¹H NMR (400 MHz, DMSO-d6) δ 8.30 (s, 1H), 8.08 (br.s, 2H), 7.95 (s, 1H), 7.25-7.19 (m, 2H), 7.11 (s, 1H), 7.09 (s, 1H), 6.96 (d, J=8.0 Hz, 1H), 6.77-6.74 (m, 5H), 4.30 (br.s, 4H), 3.96 (t, J=4.0 Hz, 2H), 3.58 (t, J=4.0 Hz, 2H), 3.27 (s, 3H), 2.99 (br.s, 4H), 2.33 (s, 3H), 2.28 (s, 3H) ppm. MS: M/e 622 (M+1)⁺

### Compound A76: 2-(5-amino-2-(furan-2-yl)-7H-pyrazolo[4,3-e][1,2,4]triazolo[1,5-c]pyrimidin-7-yl)-1-(4-(4-(3-morpholinopropoxy)phenyl)piperazin-1-yl)-2-phenylpropan-1-one

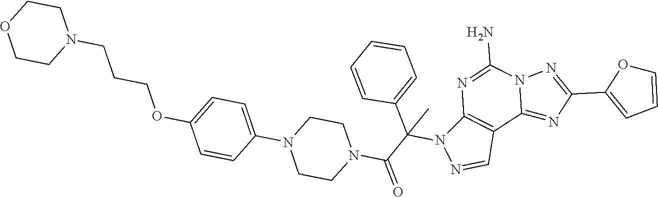 [SMILES: CC(C(=O)N1CCN(c2ccc(OCCCN3CCOCC3)cc2)CC1)(c1ccccc1)n1ncc2c1nc(N)n1nc(-c3ccco3)nc21]

A mixture of 4-(3-(4-(piperazin-1-yl)phenoxy)propyl)morpholine (100 mg, 0.33 mmol), 2-(5-amino-2-(furan-2-yl)-7H-pyrazolo[4,3-e][1,2,4]triazolo[1,5-c]pyrimidin-7-yl)-2-phenylpropanoic acid (100 mg, 0.26 mmol), HATU (110 mg, 0.3 mmol) and DIEA (100 mg, 0.78 mmol) in DMF (10 mL) was stirred overnight. The reaction mixture was poured into H₂O (30 mL) and extracted with EtOAc (50 mL×3). The combined organic layers were washed with brine, dried over Na₂SO₄, concentrated and purified by column chromatography (DCM/MeOH=0˜5% MeOH) to give the target compound (13.5 mg, 6%). ¹H NMR (400 MHz, DMSO-d6) δ 8.40 (s, 1H), 8.17 (br, 2H), 8.04 (s, 1H), 7.46-7.32 (m, 4H), 7.31-7.25 (m, 2H), 6.86-6.77 (m, 5H), 3.95 (t, J=6.3 Hz, 2H), 3.88-3.71 (m, 2H), 3.66-3.58 (m, 4H), 3.39 (s, 1H), 3.22-2.91 (m, 4H), 2.50-2.36 (m, 10H), 1.93-1.84 (m, 2H) ppm. MS: M/e 677 (M+1)⁺.

### Compound A76a: (S)-2-(5-amino-2-(furan-2-yl)-7H-pyrazolo[4,3-e][1,2,4]triazolo[1,5-c]pyrimidin-7-yl)-1-(4-(4-(3-morpholinopropoxy)phenyl)piperazin-1-yl)-2-phenylpropan-1-one

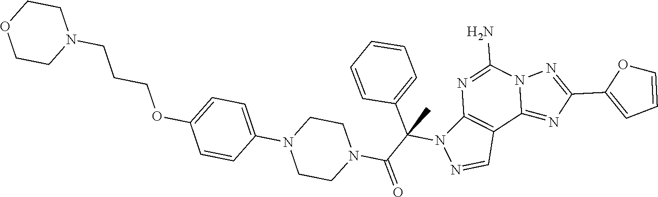 [SMILES: C[C@@](C(=O)N1CCN(c2ccc(OCCCN3CCOCC3)cc2)CC1)(c1ccccc1)n1ncc2c1nc(N)n1nc(-c3ccco3)nc21]

A mixture of 4-(3-(4-(piperazin-1-yl)phenoxy)propyl)morpholine (45 mg, 0.15 mmol), (S)-2-(5-amino-2-(furan-2-yl)-7H-pyrazolo[4,3-e][1,2,4]triazolo[1,5-c]pyrimidin-7-yl)-2-phenylpropanoic acid (50 mg, 0.13 mmol), HATU (55 mg, 0.15 mmol) and DIEA (50 mg, 0.39 mmol) in DMF (5 mL) was stirred overnight. The reaction mixture was poured into H₂O (20 mL) and extracted with EtOAc (40 mL×3). The combined organic layers were washed with brine, dried over Na₂SO₄, concentrated and purified by column chromatography (DCM/MeOH=0˜5% MeOH) to give the target compound (34 mg, 38.6%) as a white solid. ¹H NMR (400 MHz, DMSO-d6) δ 8.31 (s, 1H), 8.09 (br, 2H), 7.95 (d, J=0.9 Hz, 1H), 7.37-7.27 (m, 3H), 7.24 (d, J=3.4 Hz, 1H), 7.22-7.14 (m, 2H), 6.76-6.70 (m, 5H), 3.86 (t, J=6.3 Hz, 2H), 3.79-3.62 (m, 2H), 3.60-3.49 (m, 5H), 3.11-2.86 (m, 4H), 2.41-2.26 (m, 10H), 1.78 (dd, J=13.4, 7.0 Hz, 2H) ppm. MS: M/e 677 (M+1)⁺.

### Compound A76b: (R)-2-(5-amino-2-(furan-2-yl)-7H-pyrazolo[4,3-e][1,2,4]triazolo[1,5-c]pyrimidin-7-yl)-1-(4-(4-(3-morpholinopropoxy)phenyl)piperazin-1-yl)-2-phenylpropan-1-one

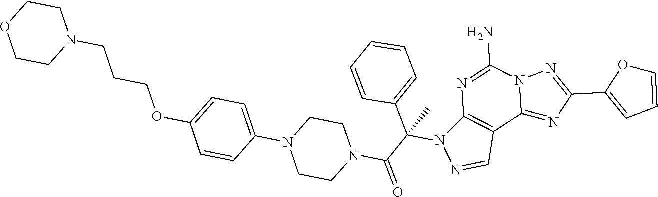 [SMILES: C[C@](C(=O)N1CCN(c2ccc(OCCCN3CCOCC3)cc2)CC1)(c1ccccc1)n1ncc2c1nc(N)n1nc(-c3ccco3)nc21]

A mixture of 4-(3-(4-(piperazin-1-yl)phenoxy)propyl)morpholine (45 mg, 0.15 mmol), (R)-2-(5-amino-2-(furan-2-yl)-7H-pyrazolo[4,3-e][1,2,4]triazolo[1,5-c]pyrimidin-7-yl)-2-phenylpropanoic acid (50 mg, 0.13 mmol), HATU (55 mg, 0.15 mmol) and DIEA (50 mg, 0.39 mmol) in DMF (5 mL) was stirred overnight. The reaction mixture was poured into H₂O (20 mL) and extracted with EtOAc (40 mL×3). The combined organic layers were washed with brine, dried over Na₂SO₄, concentrated and purified by column chromatography (DCM/MeOH=0˜5% MeOH) to give the target compound (26 mg, 29.5%) as a white solid. ¹H NMR (400 MHz, DMSO-d6) δ 8.31 (s, 1H), 8.08 (br, 2H), 7.95 (s, 1H), 7.36-7.27 (m, 3H), 7.24 (d, J=3.4 Hz, 1H), 7.22-7.15 (m, 2H), 6.77-6.68 (m, 5H), 3.86 (t, J=6.3 Hz, 3H), 3.79-3.62 (m, 2H), 3.61-3.50 (m, 5H), 3.09-2.84 (m, 4H), 2.41-2.25 (m, 10H), 1.85-1.73 (m, 2H) ppm. MS: M/e 677 (M+1)⁺.

### Compound A77: 2-(5-amino-2-(furan-2-yl)-7H-pyrazolo[4,3-e][1,2,4]triazolo[1,5-c]pyrimidin-7-yl)-1-(4-(3-methoxyphenyl)piperazin-1-yl)-2-phenylpropan-1-one

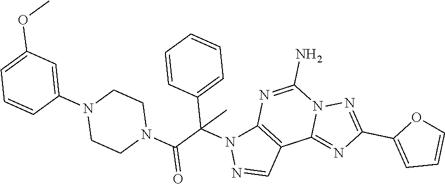 [SMILES: COc1cccc(N2CCN(C(=O)C(C)(c3ccccc3)n3ncc4c3nc(N)n3nc(-c5ccco5)nc43)CC2)c1]

A mixture of 2-(5-amino-2-(furan-2-yl)-7H-pyrazolo[4,3-e][1,2,4]triazolo[1,5-c]pyrimidin-7-yl)-2-phenylpropanoic acid (50 mg, 0.128 mmol), 1-(3-methoxyphenyl)piperazine hydrochloride (29.5 mg, 0.128 mmol), HATU (58.3 mg, 0.154 mmol) and DIPEA (33 mg, 0.256 mmol) in DMF (4 mL) was stirred overnight. The reaction mixture was poured into H₂O (10 mL) and extracted with EtOAc (10 mL×3). The combined organic layers were washed with brine, dried over Na₂SO₄, concentrated and purified by prep-TLC (petroleum ether/EtOAc=1:2) to give the target compound (20 mg, 27.7%). ¹H NMR (400 MHz, DMSO-d6) δ 8.31 (s, 1H), 8.07 (s, 2H), 7.95 (s, 1H), 7.37-7.17 (m, 6H), 7.06-7.0 (m, 1H), 6.74 (m, 1H), 6.40-6.27 (m, 3H), 3.86-3.47 (m, 2H), 3.64 (s, 3H), 3.21-2.84 (m, 4H), 2.76-2.55 (m, 1H), 2.34 (s, 3H) ppm. MS: M/e 564 (M+1)⁺.

### Compound A78: 2-(5-amino-2-(furan-2-yl)-7H-pyrazolo[4,3-e][1,2,4]triazolo[1,5-c]pyrimidin-7-yl)-1-(4-(2-methoxyphenyl)piperazin-1-yl)-2-phenylpropan-1-one

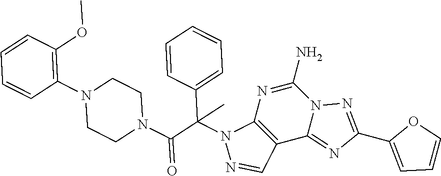 [SMILES: COc1ccccc1N1CCN(C(=O)C(C)(c2ccccc2)n2ncc3c2nc(N)n2nc(-c4ccco4)nc32)CC1]

A mixture of 2-(5-amino-2-(furan-2-yl)-7H-pyrazolo[4,3-e][1,2,4]triazolo[1,5-c]pyrimidin-7-yl)-2-phenylpropanoic acid (50 mg, 0.128 mmol), 1-(2-methoxyphenyl)piperazine hydrochloride (29.5 mg, 0.128 mmol), HATU (58.3 mg, 0.154 mmol) and DIPEA (33 mg, 0.256 mmol) in DMF (4 mL) was stirred overnight. The reaction mixture was poured into H₂O (10 mL) and extracted with EtOAc (10 mL×3). The combined organic layers were washed with brine, dried over Na₂SO₄, concentrated and purified by prep-TLC (petroleum ether/EtOAc=1:2) to give the target compound (23 mg, 31.9%). ¹H NMR (400 MHz, DMSO-d6) δ 8.32 (d, J=15.4 Hz, 1H), 8.09 (s, 2H), 7.98-7.93 (m, 1H), 7.37-7.17 (m, 6H), 6.97-6.82 (m, 2H), 6.81-6.71 (m, 2H), 6.69-6.62 (m, 1H), 3.89-3.58 (m, 2H), 3.68 (s, 3H), 3.14-2.78 (m, 4H), 2.45-2.17 (m, 2H), 2.34 (s, 3H) ppm. MS: M/e 564 (M+1)⁺.

### Compound A79: 2-(5-amino-2-(furan-2-yl)-7H-pyrazolo[4,3-e][1,2,4]triazolo[1,5-c]pyrimidin-7-yl)-2-phenyl-1-(4-(4-(3-(pyrrolidin-1-yl)propoxy)phenyl)piperazin-1-yl)propan-1-one

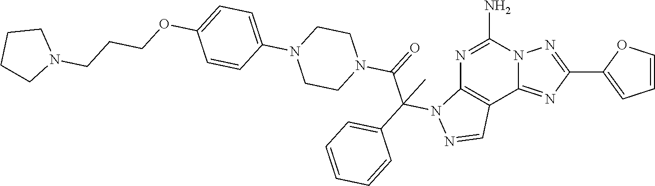 [SMILES: CC(C(=O)N1CCN(c2ccc(OCCCN3CCCC3)cc2)CC1)(c1ccccc1)n1ncc2c1nc(N)n1nc(-c3ccco3)nc21]

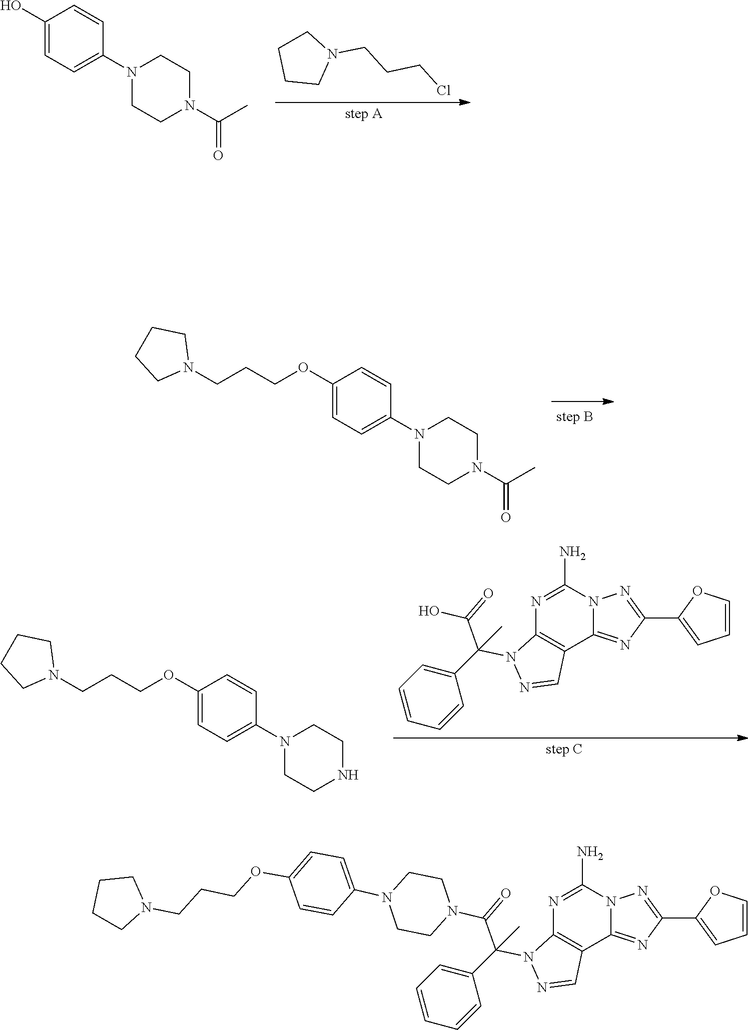

### Step A: 1-(4-(4-(3-(pyrrolidin-1l-yl)propoxy)phenyl)piperazin-1-yl)ethan-1-one

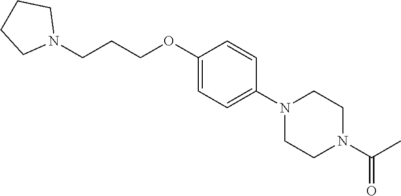 [SMILES: CC(=O)N1CCN(c2ccc(OCCCN3CCCC3)cc2)CC1]

A mixture of 1-(4-(4-hydroxyphenyl)piperazin-1-yl)ethan-1-one (1.5 g, 6.8 mmol), 1-(3-chloropropyl)pyrrolidine (1.0 g, 6.8 mmol) and Cs₂CO₃ (4.4 g, 13.5 mmol) in DMF (20 ml) was stirred at 60° C. for 15 h. After completion, the reaction mixture was poured into water (30 ml) and then extracted with DCM (20 ml*3). The combined organic layer was washed with water (20 ml), dried over Na₂SO₄ and then concentrated to remove solvent. The residue was slurried with MTBE & PE and then filtered to afford 1-(4-(4-(3-(pyrrolidin-1-yl)propoxy)phenyl)piperazin-1-yl)ethan-1-one (1.8 g, 80%) as a white solid. MS: M/e 332 (M+1)⁺

### Step B: 1-(4-(3-(pyrrolidin-1-yl)propoxy)phenyl)piperazine

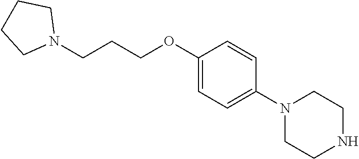 [SMILES: c1cc(N2CCNCC2)ccc1OCCCN1CCCC1]

A solution of 1-(4-(4-(3-(pyrrolidin-1-yl)propoxy)phenyl)piperazin-1-yl)ethan-1-one (1.65 g, 5.0 mmol) in conc. HCl (20 mL) and H₂O (20 ml) was stirred at 70° C. for 3 h. After completion, the reaction mixture was basified with NaOH to pH=9˜10, which was then extracted with DCM (25 ml*3). The combined organic layer was dried over Na₂SO₄, filtered and then concentrated under reduced pressure to afford 1-(4-(3-(pyrrolidin-1-yl)propoxy)phenyl)piperazine (1.5 g, 100%) as a yellow solid. MS: M/e 290 (M+1)⁺

### Step C: 2-(5-amino-2-(furan-2-yl)-7H-pyrazolo[4,3-e][1,2,4]triazolo[1,5-c]pyrimidin-7-yl)-2-phenyl-1-(4-(4-(3-(pyrrolidin-1-yl)propoxy)phenyl)piperazin-1-yl)propan-1-one

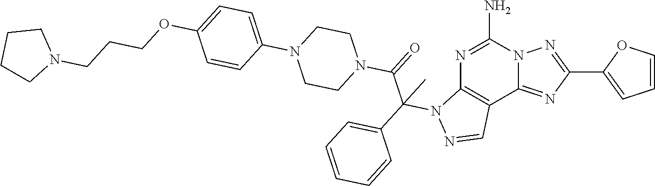 [SMILES: CC(C(=O)N1CCN(c2ccc(OCCCN3CCCC3)cc2)CC1)(c1ccccc1)n1ncc2c1nc(N)n1nc(-c3ccco3)nc21]

To a stirred solution of 2-(5-amino-2-(furan-2-yl)-7H-pyrazolo[4,3-e][1,2,4]triazolo[1,5-c]pyrimidin-7-yl)-2-phenylpropanoic acid (100 mg, 0.26 mmol), HATU (108 mg, 0.28 mmol) and DIEA (100 mg, 0.78 mmol) in THF (15 ml) was added 1-(4-(3-(pyrrolidin-1-yl)propoxy)phenyl)piperazine (74 mg, 0.26 mmol). The reaction mixture was stirred at r.t for 15 h. After completion, the reaction mixture was diluted with EA (30 ml) and then washed with H₂O (15 ml*2). The organic layer was dried over Na₂SO₄, filtered and then concentrated under reduced pressure to afford a residue. The residue was purified by prep-TLC with DCM:MeOH (20:1) to the product (11.3 mg). ¹H NMR (400 MHz, DMSO-d6) δ 8.31 (s, 1H), 8.08 (s, 2H), 7.95 (s, 1H), 7.33-7.18 (m, 6H), 6.74-6.72 (m, 5H), 3.87 (t, J=4 Hz, 2H), 3.80-3.52 (m, 2H), 3.33-3.31 (m, 2H), 3.23-2.62 (m, 6H), 2.47-2.35 (m, 4H), 2.33 (s, 3H), 1.85-1.78 (m, 2H), 1.71-1.58 (m, 4H) ppm. MS: M/e 661 (M+1)⁺.

### Compound A79a: (S)-2-(5-amino-2-(furan-2-yl)-7H-pyrazolo[4,3-e][1,2,4]triazolo[1,5-c]pyrimidin-7-yl)-2-phenyl-1-(4-(4-(3-(pyrrolidin-1-yl)propoxy)phenyl)piperazin-1-yl)propan-1-one

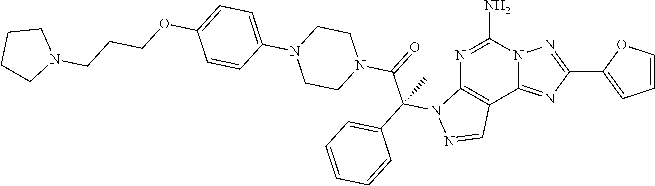 [SMILES: C[C@@](C(=O)N1CCN(c2ccc(OCCCN3CCCC3)cc2)CC1)(c1ccccc1)n1ncc2c1nc(N)n1nc(-c3ccco3)nc21]

To a stirred solution of (S)-2-(5-amino-2-(furan-2-yl)-7H-pyrazolo[4,3-e][1,2,4]triazolo[1,5-c]pyrimidin-7-yl)-2-phenylpropanoic acid (50 mg, 0.13 mmol), HATU (54 mg, 0.14 mmol) and DIEA (50 mg, 0.39 mmol) in THF (10 ml) was added 1-(4-(3-(pyrrolidin-1-yl)propoxy)phenyl)piperazine (37 mg, 0.13 mmol). The reaction mixture was stirred at rt for 15 h. After completion, the reaction mixture was diluted with EA (30 ml) and then washed with H₂O (15 ml*2). The organic layer was dried over Na₂SO₄, filtered and then concentrated under reduced pressure to afford a residue. The residue was purified by prep-TLC with DCM:MeOH (20:1) to afford the product (54.9 mg, 65%) as a white solid. ¹H NMR (400 MHz, DMSO-d6) δ 8.31 (s, 1H), 8.08 (s, 2H), 7.96 (s, 1H), 7.33-7.18 (m, 6H), 6.83-6.68 (m, 5H), 3.92 (t, J=4 Hz, 2H), 3.83-3.60 (m, 2H), 3.58-3.45 (m, 3H), 3.25-3.18 (m, 2H), 3.10-2.84 (m, 6H), 2.68-2.61 (m, 1H), 2.33 (s, 3H), 2.10-1.92 (m, 4H), 1.90-1.79 (m, 2H) ppm. MS: M/e 661 (M+1)⁺.

### Compound A79b: (R)-2-(5-amino-2-(furan-2-yl)-7H-pyrazolo[4,3-e][1,2,4]triazolo[1,5-c]pyrimidin-7-yl)-2-phenyl-1-(4-(4-(3-(pyrrolidin-1-yl)propoxy)phenyl)piperazin-1-yl)propan-1-one

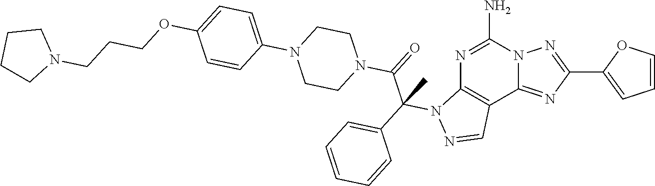 [SMILES: C[C@](C(=O)N1CCN(c2ccc(OCCCN3CCCC3)cc2)CC1)(c1ccccc1)n1ncc2c1nc(N)n1nc(-c3ccco3)nc21]

To a stirred solution of (R)-2-(5-amino-2-(furan-2-yl)-7H-pyrazolo[4,3-e][1,2,4]triazolo[1,5-c]pyrimidin-7-yl)-2-phenylpropanoic acid (50 mg, 0.13 mmol), HATU (54 mg, 0.14 mmol) and DIEA (50 mg, 0.39 mmol) in THF (10 ml) was added 1-(4-(3-(pyrrolidin-1-yl)propoxy)phenyl)piperazine (37 mg, 0.13 mmol). The reaction mixture was stirred at rt for 15 h. After completion, the reaction mixture was diluted with EA (30 ml) and then washed with H₂O (15 ml*2). The organic layer was dried over Na₂SO₄, filtered and then concentrated under reduced pressure to afford a residue. The residue was purified by prep-TLC with DCM:MeOH (20:1) to afford the product (49.2 mg, 58%) as a white solid. ¹H NMR (400 MHz, DMSO-d6) δ 8.31 (s, 1H), 8.08 (s, 2H), 7.95 (s, 1H), 7.33-7.18 (m, 6H), 6.79-6.71 (m, 5H), 3.92 (t, J=4 Hz, 2H), 3.83-3.60 (m, 2H), 3.58-3.42 (m, 3H), 3.25-3.17 (m, 2H), 3.08-2.85 (m, 6H), 2.70-2.61 (m, 1H), 2.33 (s, 3H), 2.10-1.93 (m, 4H), 1.90-1.80 (m, 2H) ppm. MS: M/e 661 (M+1)⁺.

### Compound A80: 2-(5-amino-2-(furan-2-yl)-7H-pyrazolo[4,3-e][1,2,4]triazolo[1,5-c]pyrimidin-7-yl)-1-(4-(4-(2-morpholinoethoxy)phenyl)piperazin-1-yl)-2-phenylpropan-1-one

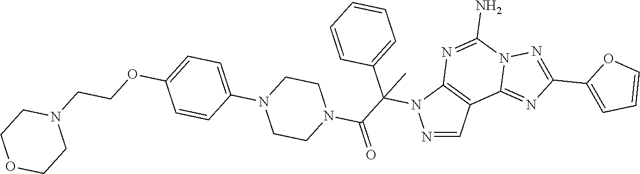 [SMILES: CC(C(=O)N1CCN(c2ccc(OCCN3CCOCC3)cc2)CC1)(c1ccccc1)n1ncc2c1nc(N)n1nc(-c3ccco3)nc21]

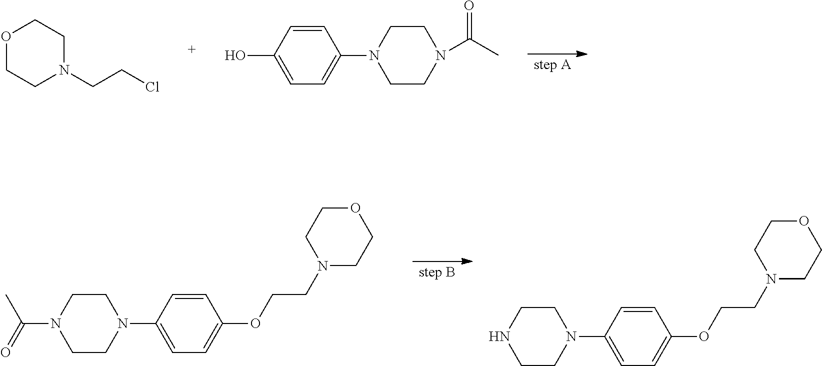 [SMILES: ClCCN1CCOCC1 ; CC(=O)N1CCN(c2ccc(O)cc2)CC1 ; CC(=O)N1CCN(c2ccc(OCCN3CCOCC3)cc2)CC1 ; c1cc(N2CCNCC2)ccc1OCCN1CCOCC1]

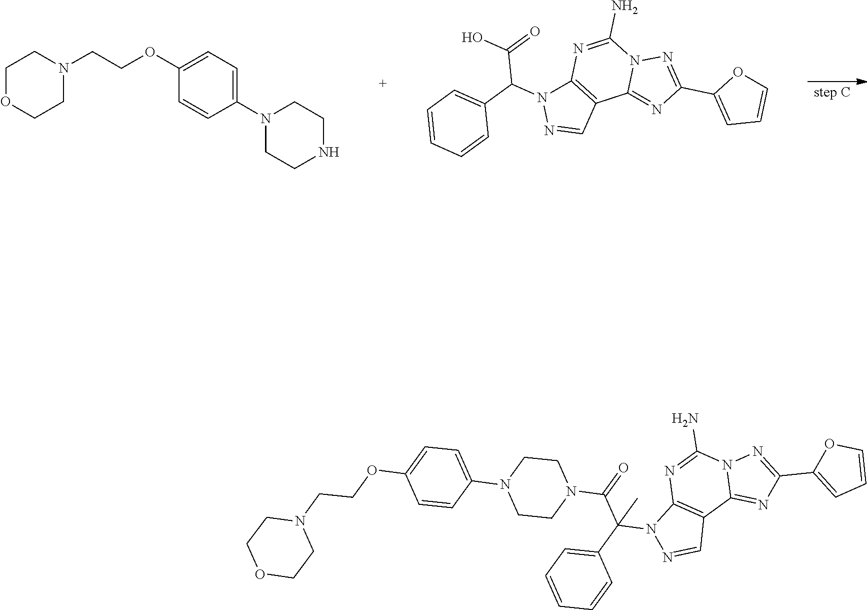 [SMILES: c1cc(N2CCNCC2)ccc1OCCN1CCOCC1 ; Nc1nc2c(cnn2C(C(=O)O)c2ccccc2)c2nc(-c3ccco3)nn12 ; CC(C(=O)N1CCN(c2ccc(OCCN3CCOCC3)cc2)CC1)(c1ccccc1)n1ncc2c1nc(N)n1nc(-c3ccco3)nc21]

### Step A: 1-(4-(4-(2-morpholinoethoxy)phenyl)piperazin-1-yl)ethan-1-one

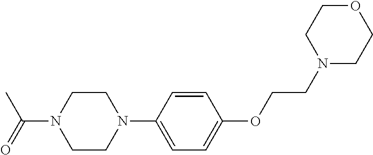 [SMILES: CC(=O)N1CCN(c2ccc(OCCN3CCOCC3)cc2)CC1]

A mixture of 1-(4-(4-hydroxyphenyl)piperazin-1-yl)ethan-1-one (5.5 g, 25 mmol), 4-(2-chloroethyl)morpholine (4.5 g, 25 mmol) and Cs₂CO₃ (12 g, 37 mmol) in DMF (150 mL) was stirred overnight. The mixture was poured into H₂O (150 mL) and extracted with DCM (150 mL×3). The combined organic layers were washed with brine, dried over Na₂SO₄, concentrated to give 1-(4-(4-(2-morpholinoethoxy)phenyl)piperazin-1-yl)ethan-1-one (4.5 g, crude) as a yellow solid. MS: M/e 334 (M+1)⁺.

### Step B: 4-(2-(4-(piperazin-1-yl)phenoxy)ethyl)morpholine

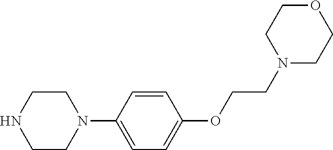 [SMILES: c1cc(N2CCNCC2)ccc1OCCN1CCOCC1]

The mixture of 1-(4-(4-(2-morpholinoethoxy)phenyl)piperazin-1-yl)ethan-1-one (1.5 g, crude) and HCl (6N, 20 mL) was stirred for 3 hours at 60° C. Then the reaction was acidified to pH=6˜7 with aq. NaOH and extracted with DCM (50 mL×3). The combined organic layers were washed with brine, dried over Na₂SO₄, concentrated to give 4-(3-(4-(piperazin-1-yl)phenoxy)propyl)morpholine (1.0 g, crude) as a deep red solid. MS: M/e 292 (M+1)⁺

### Step C: 2-(5-amino-2-(furan-2-yl)-7H-pyrazolo[4,3-e][1,2,4]triazolo[1,5-c]pyrimidin-7-yl)-1-(4-(4-(2-morpholinoethoxy)phenyl)piperazin-1-yl)-2-phenylpropan-1-one

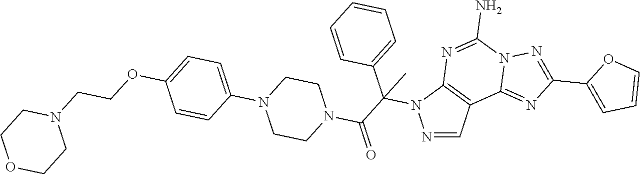 [SMILES: CC(C(=O)N1CCN(c2ccc(OCCN3CCOCC3)cc2)CC1)(c1ccccc1)n1ncc2c1nc(N)n1nc(-c3ccco3)nc21]

To a stirred solution of 2-(5-amino-2-(furan-2-yl)-7H-pyrazolo[4,3-e][1,2,4]triazolo[1,5-c]pyrimidin-7-yl)-2-phenylpropanoic acid (80 mg, 0.21 mmol), HATU (88 mg, 0.23 mmol) and DIPEA (80 mg, 0.62 mmol) in THF (10 ml) was added 4-(2-(4-(piperazin-1-yl)phenoxy)ethyl)morpholine (80 mg, 0.26 mmol). The reaction mixture was stirred at rt for 16 h. After completion, the reaction mixture was diluted with EA (30 ml) and then washed with H₂O (15 ml*2). The organic layer was dried over Na₂SO₄, filtered and then concentrated under reduced pressure to afford a residue. The residue was purified by prep-TLC with DCM:MeOH (20:1) to afford the product (12.4 mg). ¹H NMR (400 MHz, DMSO-d6) δ 8.31 (s, 1H), 8.08 (s, 2H), 7.95 (s, 1H), 7.38-7.26 (m, 3H), 7.24 (d, J=4 Hz, 1H), 7.20 (d, J=8 Hz, 2H), 6.80-6.69 (m, 5H), 3.94 (t, J=4 Hz, 2H), 3.87-3.50 (m, 6H), 3.33-3.31 (m, 1H), 3.11-2.82 (m, 4H), 2.65-2.63 (m, 1H), 2.62-2.55 (t, J=4 Hz, 2H), 2.45-2.42 (m, 4H), 2.33 (s, 3H) ppm. MS: M/e 663 (M+1)⁺.

### Compound A80a: (S)-2-(5-amino-2-(furan-2-yl)-7H-pyrazolo[4,3-e][1,2,4]triazolo[1,5-c]pyrimidin-7-yl)-1-(4-(4-(2-morpholinoethoxy)phenyl)piperazin-1-yl)-2-phenylpropan-1-one

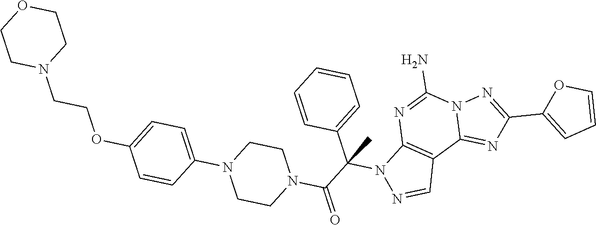 [SMILES: C[C@@](C(=O)N1CCN(c2ccc(OCCN3CCOCC3)cc2)CC1)(c1ccccc1)n1ncc2c1nc(N)n1nc(-c3ccco3)nc21]

A mixture of 4-(2-(4-piperazin-1-yl)phenoxy)ethyl)morpholine (50 mg, 0.17 mmol), (S)-2-(5-amino-2-(furan-2-yl)-7H-pyrazolo[4,3-e][1,2,4]triazolo[1,5-c]pyrimidin-7-yl)-2-phenylpropanoic acid (50 mg, 0.13 mmol), HATU (55 mg, 0.15 mmol) and DIPEA (50 mg, 0.39 mmol) in DMF (5 mL) was stirred overnight. The reaction mixture was poured into H₂O (20 mL) and extracted with EtOAc (40 mL×3). The combined organic layers were washed with brine, dried over Na₂SO₄, concentrated and purified by column chromatography (DCM/MeOH=0˜5% MeOH) to give the target compound (18 mg, 20.9%). ¹H NMR (400 MHz, DMSO-d6) δ 8.31 (s, 1H), 8.07 (br.s, 2H), 7.95 (s, 1H), 7.36-7.26 (m, 3H), 7.24 (d, J=2.9 Hz, 1H), 7.22-7.14 (m, 2H), 6.80-6.67 (m, 5H), 3.94 (t, J=5.8 Hz, 2H), 3.81-3.63 (m, 1H), 3.59-3.49 (m, 4H), 3.30 (s, 1H), 2.95 (s, 4H), 2.60 (t, J=5.8 Hz, 2H), 2.46-2.37 (m, 5H), 2.36-2.25 (m, 4H) ppm. MS: M/e 663 (M+1)⁺.

### Compound A80b: (R)-2-(5-amino-2-(furan-2-yl)-7H-pyrazolo[4,3-e][1,2,4]triazolo[1,5-c]pyrimidin-7-yl)-1-(4-(4-(2-morpholinoethoxy)phenyl)piperazin-1-yl)-2-phenylpropan-1-one

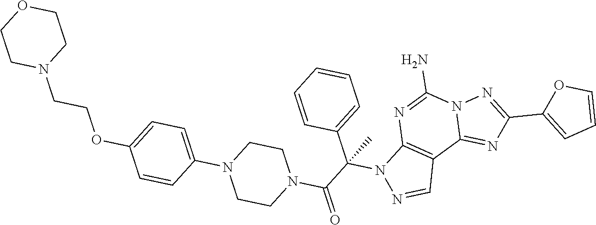 [SMILES: C[C@](C(=O)N1CCN(c2ccc(OCCN3CCOCC3)cc2)CC1)(c1ccccc1)n1ncc2c1nc(N)n1nc(-c3ccco3)nc21]

A mixture of 4-(2-(4-(piperazin-1-yl)phenoxy)ethyl)morpholine (45 mg, 0.15 mmol), (R)-2-(5-amino-2-(furan-2-yl)-7H-pyrazolo[4,3-e][1,2,4]triazolo[1,5-c]pyrimidin-7-yl)-2-phenylpropanoic acid (50 mg, 0.13 mmol), HATU (55 mg, 0.15 mmol) and DIPEA (50 mg, 0.39 mmol) in DMF (5 mL) was stirred overnight. The reaction mixture was poured into H₂O (20 mL) and extracted with EtOAc (40 mL×3). The combined organic layers were washed with brine, dried over Na₂SO₄, concentrated and purified by column chromatography (DCM/MeOH=0˜5% MeOH) to give the target compound (10.6 mg, 12.3%). ¹H NMR (400 MHz, DMSO-d6) δ 8.31 (s, 1H), 8.08 (br.s, 2H), 7.95 (s, 1H), 7.37-7.26 (m, 3H), 7.24 (d, J=3.4 Hz, 1H), 7.22-7.12 (m, 2H), 6.80-6.69 (m, 5H), 3.94 (t, J=5.8 Hz, 2H), 3.82-3.61 (m, 1H), 3.59-3.50 (m, 4H), 3.30 (s, 1H), 3.12-2.80 (m, 4H), 2.60 (t, J=5.8 Hz, 2H), 2.47-2.38 (m, 5H), 2.33 (s, 4H) ppm. MS: M/e 663 (M+1)⁺.

### Compound A81: 2-(5-amino-2-(furan-2-yl)-7H-pyrazolo[4,3-e][1,2,4]triazolo[1,5-c]pyrimidin-7-yl)-1-(4-(4-(2-(4-methylpiperazin-1-yl)ethoxy)phenyl)piperazin-1-yl)-2-phenylpropan-1-one

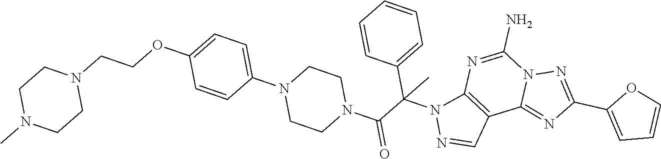 [SMILES: CN1CCN(CCOc2ccc(N3CCN(C(=O)C(C)(c4ccccc4)n4ncc5c4nc(N)n4nc(-c6ccco6)nc54)CC3)cc2)CC1]

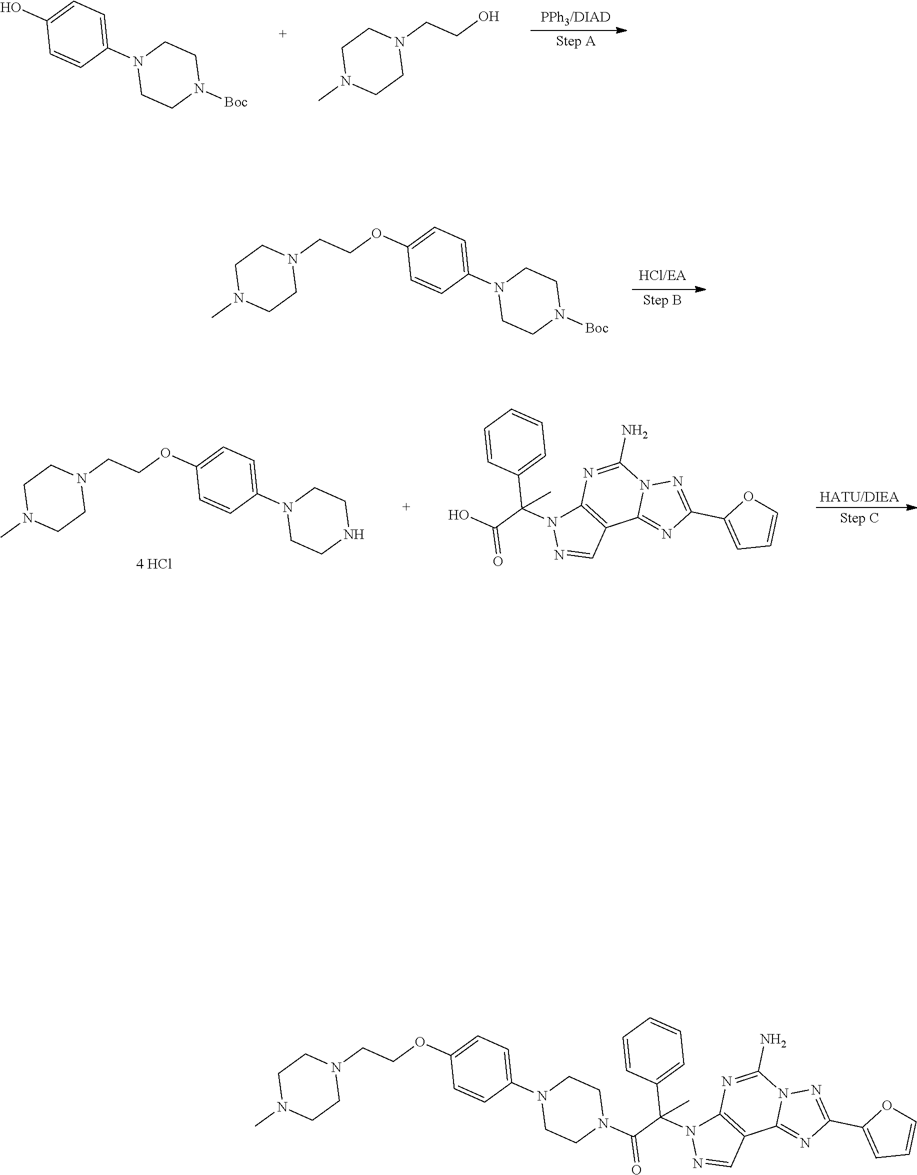

### Step A: tert-butyl 4-(4-(2-(4-methylpiperazin-1-yl)ethoxy)phenyl)piperazine-1-carboxylate

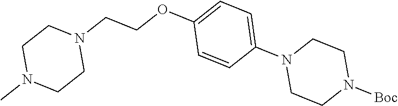 [SMILES: CN1CCN(CCOc2ccc(N3CCN(C(=O)OC(C)(C)C)CC3)cc2)CC1]

DIAD (2.6 g, 13 mmol) was added dropwise to a solution of tert-butyl 4-(4-hydroxyphenyl)piperazine-1-carboxylate (3 g, 11 mmol), 2-(4-methylpiperazin-1-yl)ethan-1-ol (2.3 g, 16 mmol) and PPh₃ (4.3 g, 16 mmol) in THF (50 mL). The resulting mixture was stirred at rt overnight. The solution was added with water (20 mL), extracted with ethyl acetate (30 mL) and washed with brine (20 mL). The organic layer was dried, concentrated and purified by column chromatography (DCM:MeOH=20:1) to get the desired product as a colorless oil (3.3 g, 75%). ¹H NMR (400 MHz, DMSO-d6) δ 6.90-6.81 (m, 4H), 3.98 (t, J=8.0 Hz, 2H), 3.44 (t, J=4.0 Hz, 4H), 2.94 (t, J=4.0 Hz, 4H), 2.63 (t, J=8.0 Hz, 2H), 2.46-2.30 (m, 8H), 2.13 (s, 3H), 1.41 (s, 9H) ppm. MS: M/e 405 (M+1)⁺

### Step B: 1-methyl-4-(2-(4-(piperazin-1-yl)phenoxy)ethyl)piperazine Hydrochloride

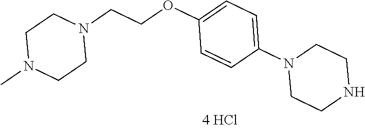 [SMILES: CN1CCN(CCOc2ccc(N3CCNCC3)cc2)CC1]

HCl/EA solution (2M, 5 mL) was added to a solution of tert-butyl 4-(4-(2-(4-methylpiperazin-1-yl)ethoxy)phenyl)piperazine-1-carboxylate (3.3 g, 8.2 mmol) in EA (20 mL). A white solid was precipitated immediately. The mixture was stirred at rt overnight. The white solid was filtered, washed with ethyl acetate (10 mL) and dried to get the product as HCl salt (3.5 g, 96%). ¹H NMR (400 MHz, DMSO-d6) δ 12.13 (br.s, 1H), 9.46 (s, 2H), 7.06-6.97 (m, 4H), 5.84 (br.s, 4H), 4.38 (d, J=4.0 Hz, 2H), 3.82-3.53 (m, 10H), 3.32-3.23 (m, 8H), 2.82 (s, 3H) ppm. MS: M/e 305 (M+1)⁺

### Step C: 2-(5-amino-2-(furan-2-yl)-7H-pyrazolo[4,3-e][1,2,4]triazolo[1,5-c]pyrimidin-7-yl)-1-(4-(4-(2-(4-methylpiperazin-1-yl)ethoxy)phenyl)piperazin-1-yl)-2-phenylpropan-1-one

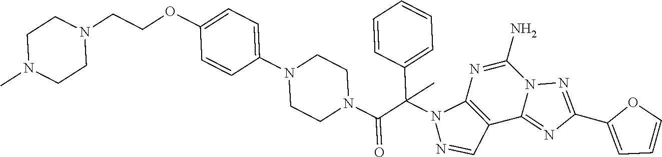 [SMILES: CN1CCN(CCOc2ccc(N3CCN(C(=O)C(C)(c4ccccc4)n4ncc5c4nc(N)n4nc(-c6ccco6)nc54)CC3)cc2)CC1]

A mixture of 2-(5-amino-2-(furan-2-yl)-7H-pyrazolo[4,3-e][1,2,4]triazolo[1,5-c]pyrimidin-7-yl)-2-phenylpropanoic acid (50 mg, 0.13 mmol), 1-methyl-4-(2-(4-(piperazin-1-yl)phenoxy)ethyl)piperazine hydrochloride (72 mg, 0.16 mmol), HATU (61 mg, 0.16 mmol) and DIPEA (34 mg, 0.26 mmol) in DMF (5 mL) was stirred at rt for 2 hrs. The solution was added with water (10 mL), extracted with ethyl acetate (10 mL) and washed with brine (10 mL). The organic layer was dried, concentrated and purified by column chromatography (DCM:MeOH=15:1) to get the desired product (40 mg, 46%). ¹H NMR (400 MHz, DMSO-d6) δ 8.31 (s, 1H), 8.08 (br.s, 2H), 7.95 (s, 1H), 7.31-7.18 (m, 6H), 6.75-6.70 (m, 5H), 3.94 (t, J=8.0 Hz, 2H), 3.70 (br.s, 2H), 2.94 (br.s, 4H), 2.64-2.51 (m, 9H), 2.49-2.30 (m, 9H) ppm. MS: M/e 676 (M+1)⁺

### Compound A82: 2-(5-amino-2-(furan-2-yl)-7H-pyrazolo[4,3-e][1,2,4]triazolo[1,5-c]pyrimidin-7-yl)-1-(4-(4-(3-(4-methylpiperazin-1-yl)propoxy)phenyl)piperazin-1-yl)-2-phenylpropan-1-one

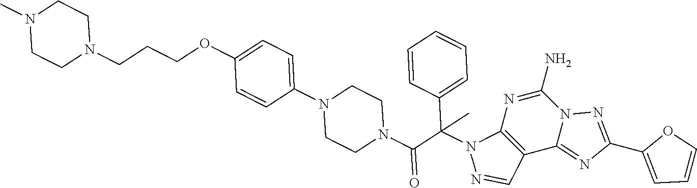 [SMILES: CN1CCN(CCCOc2ccc(N3CCN(C(=O)C(C)(c4ccccc4)n4ncc5c4nc(N)n4nc(-c6ccco6)nc54)CC3)cc2)CC1]

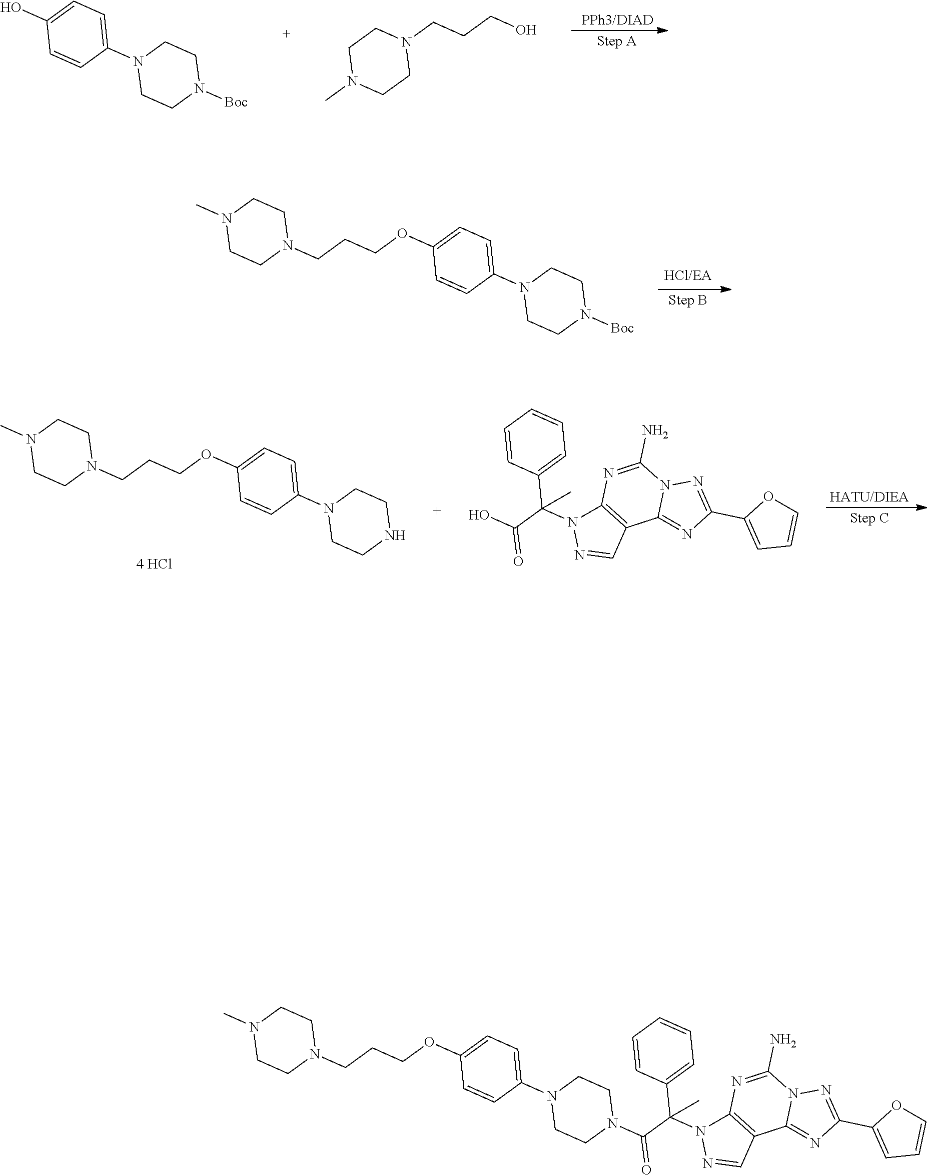

### Step A: tert-butyl 4-(4-(3-(4-methylpiperazin-1-yl)propoxy)phenyl)piperazine-1-carboxylate

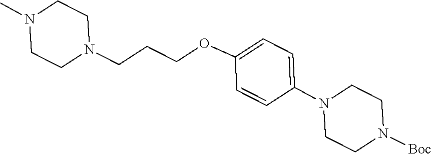 [SMILES: CN1CCN(CCCOc2ccc(N3CCN(C(=O)OC(C)(C)C)CC3)cc2)CC1]

DIAD (2.6 g, 13 mmol) was added dropwise to a solution of tert-butyl 4-(4-hydroxyphenyl)piperazine-1-carboxylate (3 g, 11 mmol), 3-(4-methylpiperazin-1-yl)propan-1-ol (2.5 g, 16 mmol) and PPh₃ (4.3 g, 16 mmol) in THF (50 mL). The resulting mixture was stirred at rt overnight. The solution was added with water (20 mL), extracted with ethyl acetate (30 mL) and washed with brine (20 mL). The organic layer was dried, concentrated and purified by column chromatography (DCM:MeOH=15:1) to get the desired product as a colorless oil (3.5 g, 78%). ¹H NMR (400 MHz, DMSO-d6) δ 6.88 (d, J=8.0 Hz, 2H), 6.81 (d, J=8.0 Hz, 2H), 3.90 (t, J=8.0 Hz, 2H), 3.44 (t, J=4.0 Hz, 4H), 2.94 (t, J=4.0 Hz, 4H), 2.38-2.30 (m, 10H), 2.14 (s, 3H), 1.82-1.79 (m, 2H), 1.41 (s, 9H) ppm. MS: M/e 419 (M+1)⁺

### Step B: 1-methyl-4-(3-(4-(piperazin-1-yl)phenoxy)propyl)piperazine Hydrochloride

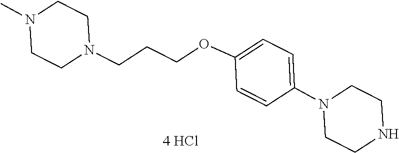 [SMILES: CN1CCN(CCCOc2ccc(N3CCNCC3)cc2)CC1]

HCl/EA solution (2M, 5 mL) was added to a solution of tert-butyl 4-(4-(3-(4-methylpiperazin-1-yl)propoxy)phenyl)piperazine-1-carboxylate (3.5 g, 8.4 mmol) in EA (20 mL). A white solid was precipitated immediately. The mixture was stirred at rt overnight and TLC showed the reaction was complete. The white solid was filtered, washed with ethyl acetate (10 mL) and dried to get the product as HCl salt (3.8 g, 100%). MS: M/e 319 (M+1)⁺

### Step C: 2-(5-amino-2-(furan-2-yl)-7H-pyrazolo[4,3-e][1,2,4]triazolo[1,5-c]pyrimidin-7-yl)-1-(4-(4-(3-(4-methylpiperazin-1-yl)propoxy)phenyl)piperazin-1-yl)-2-phenylpropan-1-one

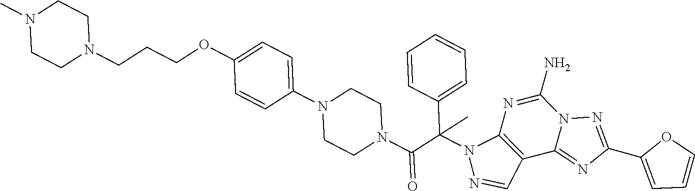 [SMILES: CN1CCN(CCCOc2ccc(N3CCN(C(=O)C(C)(c4ccccc4)n4ncc5c4nc(N)n4nc(-c6ccco6)nc54)CC3)cc2)CC1]

A mixture of 2-(5-amino-2-(furan-2-yl)-7H-pyrazolo[4,3-e][1,2,4]triazolo[1,5-c]pyrimidin-7-yl)-2-phenylpropanoic acid (50 mg, 0.13 mmol), 1-methyl-4-(3-(4-(piperazin-1-yl)phenoxy)propyl)piperazine hydrochloride (74 mg, 0.16 mmol), HATU (61 mg, 0.16 mmol) and DIPEA (34 mg, 0.26 mmol) in DMF (5 mL) was stirred at rt for 2 hrs. The solution was added with water (10 mL), extracted with ethyl acetate (10 mL) and washed with brine (10 mL). The organic layer was dried, concentrated and purified by column chromatography (DCM:MeOH=15:1) to get the desired product (35 mg, 40%). ¹H NMR (400 MHz, DMSO-d6) δ 8.31 (s, 1H), 8.08 (br.s, 2H), 7.95 (s, 1H), 7.33-7.18 (m, 6H), 6.75-6.72 (m, 5H), 3.86 (t, J=8.0 Hz, 2H), 3.69 (br.s, 2H), 2.94 (br.s, 4H), 2.67-2.50 (m, 9H), 2.45-2.33 (m, 9H), 1.80 (s, 2H) ppm. MS: M/e 690 (M+1)⁺

### Compound A83: 4-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)-N-((3-hydroxyoxetan-3-yl)methyl)benzamide

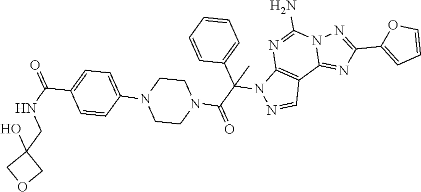 [SMILES: CC(C(=O)N1CCN(c2ccc(C(=O)NCC3(O)COC3)cc2)CC1)(c1ccccc1)n1ncc2c1nc(N)n1nc(-c3ccco3)nc21]

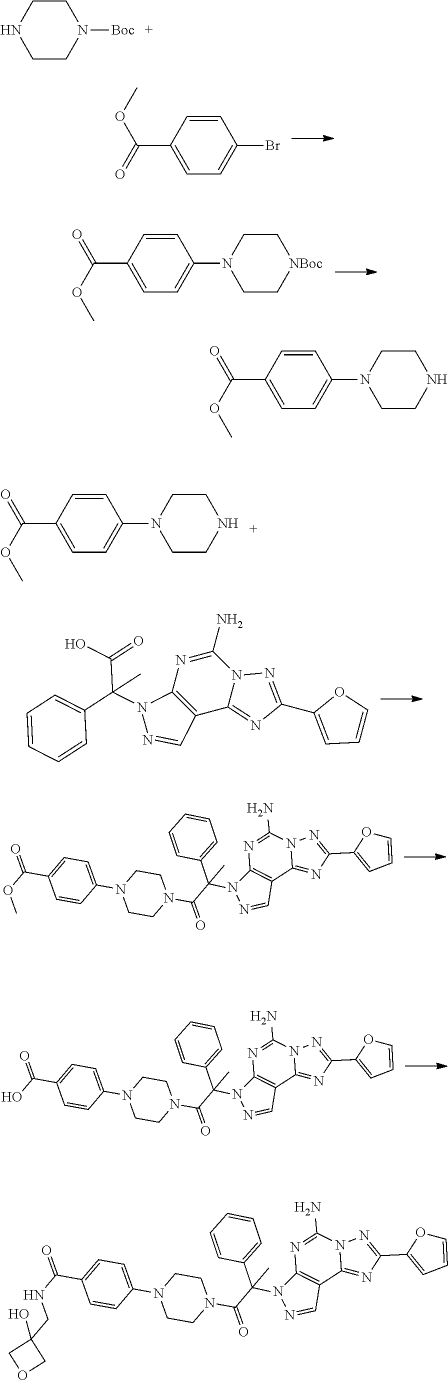

### Step A: tert-butyl 4-(4-(methoxycarbonyl)phenyl)piperazine-1-carboxylate

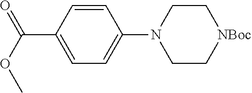 [SMILES: COC(=O)c1ccc(N2CCN(C(=O)OC(C)(C)C)CC2)cc1]

To a solution of tert-butyl piperazine-1-carboxylate (1.0 g, 4.6 mmol) and methyl 4-bromobenzoate (0.85 g, 4.6 mmol) in toluene (30 mL) was added x-phose (421 mg, 0.46 mmol), Pd(dba)₂ (438 g, 0.92 mmol) and Cs₂CO₃ (3.0 g, 9.2 mmol) and the mixture was heated at 120° C. for 2 hours. The combined organic layers were washed with brine, dried over Na₂SO₄, concentrated and purified by column chromatography (petroleum ether/EtOAc=2:1˜1:2) to give the target compound (600 mg, 40.6%) as yellow solids. MS: M/e 321 (M+1)⁺.

### Step B: methyl 4-(piperazin-1-yl)benzoate

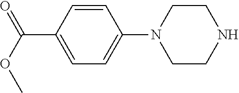 [SMILES: COC(=O)c1ccc(N2CCNCC2)cc1]

To a solution of tert-butyl 4-(4-(methoxycarbonyl)phenyl)piperazine-1-carboxylate

(600 mg, 1.9 mmol) in EA (20 mL) was added HCl/EA (20 mL, 4M), and the mixture was stirred overnight at RT. The reaction mixture filtered, the cake was collected, dried to give the target compound (400 mg, 82.2%) as a yellow solid. MS: M/e 221 (M+1)⁺.

### Step C: methyl 4-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)benzoate

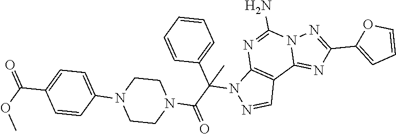 [SMILES: COC(=O)c1ccc(N2CCN(C(=O)C(C)(c3ccccc3)n3ncc4c3nc(N)n3nc(-c5ccco5)nc43)CC2)cc1]

A mixture of the product of step B (80 mg, 0.32 mmol), 2-(5-amino-2-(furan-2-yl)-7H-pyrazolo[4,3-e][1,2,4]triazolo[1,5-c]pyrimidin-7-yl)-2-phenylpropanoic acid (100 mg, 0.26 mmol), HATU (100 mg, 0.26 mmol) and DIEA (100 mg, 0.78 mmol) in DMF (10 mL) was stirred overnight at RT. The reaction mixture was poured into H₂O (20 mL) and extracted with EtOAc (50 mL×3). The combined organic layers were washed with brine, dried over Na₂SO₄, concentrated and purified by column chromatography (petroleum ether/EtOAc=1:1˜100% EtOAc) to give the target compound (100 mg, 65%) as a yellow solid. MS: M/e 592 (M+1)⁺.

### Step D: 4-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)benzoic Acid

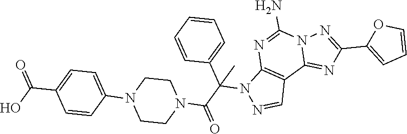 [SMILES: CC(C(=O)N1CCN(c2ccc(C(=O)O)cc2)CC1)(c1ccccc1)n1ncc2c1nc(N)n1nc(-c3ccco3)nc21]

To a stirred mixture of methyl 4-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)benzoate (100 mg, 0.17 mmol) in MeOH/H₂O (4 mL/1 mL) was added aq.NaOH (4.0 M, 1 mL). After the addition, the reaction mixture was stirred overnight. Most of solvent was removed to give the aqueous layer, then acidified to pH=3˜4 with aq.HCl and filtered, the cake was collected, dried to give the target compound (80 mg, 81.4%) as a white solid. MS: M/e 578 (M+1)⁺.

### Step E: 4-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)-N-((3-hydroxyoxetan-3-yl)methyl)benzamide

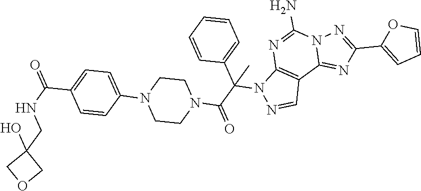 [SMILES: CC(C(=O)N1CCN(c2ccc(C(=O)NCC3(O)COC3)cc2)CC1)(c1ccccc1)n1ncc2c1nc(N)n1nc(-c3ccco3)nc21]

A mixture of the product of step D (40 mg, 0.07 mmol), 3-(aminomethyl)oxetan-3-ol (20 mg, 0.19 mmol), HATU (40 mg, 0.1 mmol) and DIEA (30 mg, 0.23 mmol) in DMF (5 mL) was stirred overnight at RT. The reaction mixture was poured into H₂O (20 mL) and extracted with EtOAc (50 mL×3). The combined organic layers were washed with brine, dried over Na₂SO₄, concentrated and purified by column chromatography (petroleum ether/EtOAc=1:1˜100% EtOAc) to give the target compound (20 mg, 43.1%) as a yellow solid. ¹H NMR (400 MHz, DMSO-d6) δ 8.32 (s, 1H), 8.30 (br, 1H), 8.08 (br, 2H), 7.96-7.93 (m, 1H), 7.69 (d, J=8.9 Hz, 2H), 7.36-7.16 (m, 6H), 6.82 (d, J=9.0 Hz, 2H), 6.74 (dd, J=3.4, 1.8 Hz, 1H), 5.85 (br, 1H), 4.45 (d, J=6.5 Hz, 2H), 4.34 (d, J=6.5 Hz, 2H), 3.83-3.56 (m, 2H), 3.50 (d, J=6.0 Hz, 2H), 3.30 (s, 1H), 3.27-3.14 (m, 2H), 3.12-2.91 (m, 2H), 2.86-2.72 (m, 1H), 2.34 (s, 3H) ppm. MS: M/e 663 (M+1)⁺.

### Compound A84: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-phenylbutan-1-one

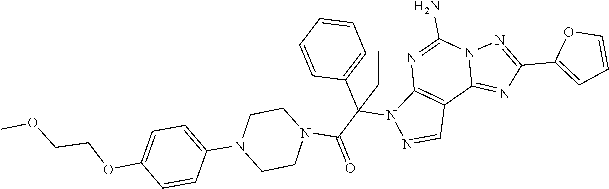 [SMILES: CCC(C(=O)N1CCN(c2ccc(OCCOC)cc2)CC1)(c1ccccc1)n1ncc2c1nc(N)n1nc(-c3ccco3)nc21]

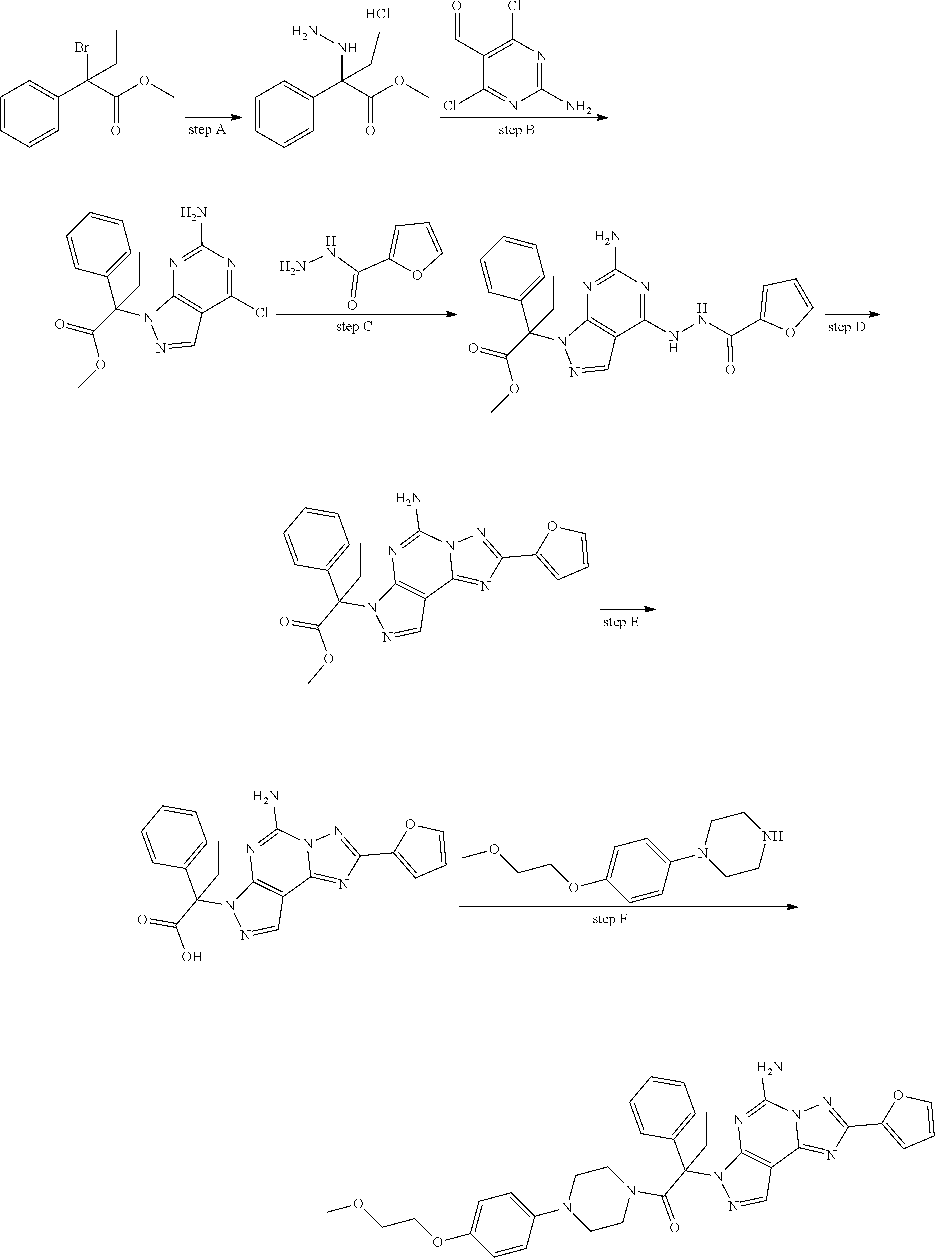

### Step A: methyl 2-hydrazinyl-2-phenylbutanoate Hydrochloride

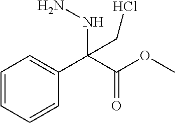

To a stirred solution of methyl 2-bromo-2-phenylbutanoate (2 g, 7.8 mmol) and K₂CO₃ (2.16 mg, 15.7 mmol) in DMF (20 ml) was added hydrazine hydrate (1.95 g, 31.2 mmol). The reaction mixture was stirred at rt for 24 h. After completion, the reaction mixture was poured into water (30 ml), which was then extracted with EA (30 ml×3). The combined organic layer was concentrated under reduced pressure to remove solvent. The residue was diluted with EA (50 ml) and then washed with H₂O (25 ml×2) to remove hydrazine hydrate. The resulting organic solution was concentrated to afford a residue. The residue was acidified with aq. HCl (4M) and then washed with EA (25 ml×2) to remove impurities which are easily soluble in EA. The aqueous solution was concentrated under reduced pressure and dried to afford the product (1.0 g, 52.6%) as a white solid, which was used directly for the next step without further purification. ¹H NMR (400 MHz, DMSO-d6) δ 7.41 (d, J=4 Hz, 4H), 7.39-7.35 (m, 1H), 3.72 (s, 3H), 2.21-2.14 (m, 2H), 0.74 (t, J=8 Hz, 3H) ppm. MS: M/e 209 (M+1)⁺.

### Step B: methyl 2-(6-amino-4-chloro-1H-pyrazolo[3,4-d]pyrimidin-1-yl)-2-phenylbutanoate

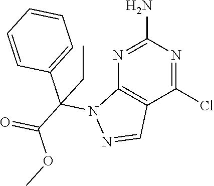 [SMILES: CCC(C(=O)OC)(c1ccccc1)n1ncc2c(Cl)nc(N)nc21]

To a stirred solution of methyl 2-hydrazinyl-2-phenylbutanoate hydrochloride (1 g, 4.09 mmol) in acetonitrile (30 ml) was added 2-amino-4,6-dichloropyrimidine-5-carbaldehyde (0.94 g, 4.90 mmol). The reaction mixture was stirred at rt for 15 h and then heated to 60° C., which was stirred at 60° C. for 2 h. After completion, the reaction mixture was filtered. The filtrate was concentrated under reduced pressure to afford a residue. The residue was purified by column chromatography with PE:EA (4:1) to afford the product (0.55 g, 39%) as a yellow solid. MS: M/e 346 (M+1)⁺.

### Step C: methyl 2-(6-amino-4-(2-(furan-2-carbonyl)hydrazinyl)-1H-pyrazolo[3,4-d]pyrimidin-1-yl)-2-phenylbutanoate

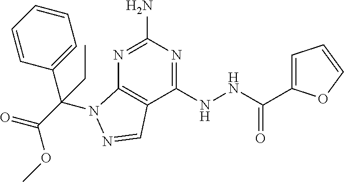 [SMILES: CCC(C(=O)OC)(c1ccccc1)n1ncc2c(NNC(=O)c3ccco3)nc(N)nc21]

A solution of methyl 2-(6-amino-4-chloro-1H-pyrazolo[3,4-d]pyrimidin-1-yl)-2-phenylbutanoate (0.55 g, 1.59 mmol), furan-2-carbohydrazide (0.20 g, 1.59 mmol) and DIEA (0.41 g, 3.18 mmol) in DMSO (15 ml) was stirred at 110° C. for 15 h. After completion, the reaction mixture was poured into H₂O (15 ml) and then extracted with EA (20 ml×3). The combined organic layers were dried over Na₂SO₄, filtered and then concentrated under reduced pressure to afford a residue. The residue was purified by column chromatography with PE:EA (1:2) to afford the product (0.57 g, 82%) as a light-yellow solid. MS: M/e 436 (M+1)⁺.

### Step D: methyl 2-(5-amino-2-(furan-2-yl)-7H-pyrazolo[4,3-e][1,2,4]triazolo[1,5-c]pyrimidin-7-yl)-2-phenylbutanoate

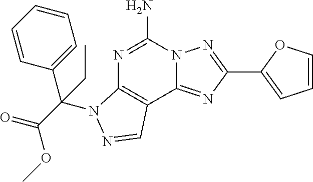 [SMILES: CCC(C(=O)OC)(c1ccccc1)n1ncc2c1nc(N)n1nc(-c3ccco3)nc21]

A solution of methyl 2-(6-amino-4-(2-(furan-2-carbonyl)hydrazinyl)-1H-pyrazolo[3,4-d]pyrimidin-1-yl)-2-phenylbutanoate (0.57 g, 1.31 mmol) in BSA (5 ml) and HMDS (5 ml) was stirred at 110° C. for 15 h. After completion, the reaction mixture was concentrated. The residue was diluted with aq. NaHCO₃(sat., 15 ml) and then extracted with EA (20 ml×3). The combined organic layers were dried over Na₂SO₄, filtered and then concentrated under reduced pressure to afford a residue. The residue was purified by column chromatography with PE:EA (10:1) to afford the product (0.28 g, 51%) as a yellow solid. MS: M/e 418 (M+1)⁺.

### Step E: 2-(5-amino-2-(furan-2-yl)-7H-pyrazolo[4,3-e][1,2,4]triazolo[1,5-c]pyrimidin-7-yl)-2-phenylbutanoic Acid

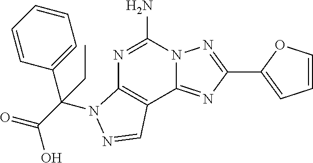 [SMILES: CCC(C(=O)O)(c1ccccc1)n1ncc2c1nc(N)n1nc(-c3ccco3)nc21]

To a stirred solution of methyl 2-(5-amino-2-(furan-2-yl)-7H-pyrazolo[4,3-e][1,2,4]triazolo[1,5-c]pyrimidin-7-yl)-2-phenylbutanoate (0.28 g, 0.67 mmol) in ethanol (15 ml) was added a solution of NaOH (0.27 g, 6.75 mmol) in H₂O (5 ml). The mixture was stirred at 70° C. for 4 h. After completion, the reaction mixture was concentrated to remove ethanol. The residue was diluted with H₂O (15 ml) and then acidified with aq. HCl (4M) to pH=3˜4. The precipitate was filtered. The filtration cake was washed with H₂O (15 ml) and then dried to afford the product (0.25 g, 92%) as a white solid. MS: M/e 404 (M+1)⁺.

### Step F: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-phenylbutan-1-one

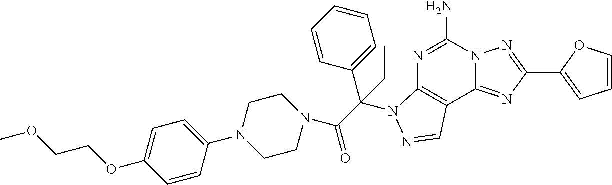 [SMILES: CCC(C(=O)N1CCN(c2ccc(OCCOC)cc2)CC1)(c1ccccc1)n1ncc2c1nc(N)n1nc(-c3ccco3)nc21]

To a stirred solution of 2-(5-amino-2-(furan-2-yl)-7H-pyrazolo[4,3-e][1,2,4]triazolo[1,5-c]pyrimidin-7-yl)-2-phenylbutanoic acid (0.12 g, 0.30 mmol), HATU (0.12 g, 0.33 mmol) and DIEA (0.12 g, 0.89 mmol) in THF (10 ml) was added 1-(4-(2-methoxyethoxy)phenyl)piperazine (70 mg, 0.30 mmol). The reaction mixture was stirred at rt for 15 h. After completion, the reaction mixture was diluted with EA (30 ml) and then washed with H₂O (15 ml×2). The organic layer was dried over Na₂SO₄, filtered and then concentrated under reduced pressure to afford a residue. The residue was purified by prep-TLC with PE:EA (1:5) to afford the product (35.9 mg). ¹H NMR (400 MHz, DMSO-d6) δ 8.35 (s, 1H), 8.07 (s, 2H), 7.95 (s, 1H), 7.44 (d, J=4 Hz, 2H), 7.38 (d, J=4 Hz, 2H), 7.34-7.22 (m, 2H), 6.77-6.65 (m, 5H), 3.94 (t, J=4 Hz, 2H), 3.56 (t, J=4 Hz, 2H), 3.51-3.40 (m, 2H), 3.26 (s, 3H), 3.15-2.86 (m, 3H), 2.77 (q, 2H), 2.70-2.63 (m, 1H), 2.60-2.51 (m, 2H), 0.63 (t, J=8 Hz, 3H) ppm. MS: M/e 622 (M+1)⁺.

### Compound A85: 2-(5-amino-2-(furan-2-yl)-7H-pyrazolo[4,3-e][1,2,4]triazolo[1,5-c]pyrimidin-7-yl)-2-phenyl-1-(4-(4-(3-(piperidin-1-yl)propoxy)phenyl)piperazin-1-yl)propan-1-one

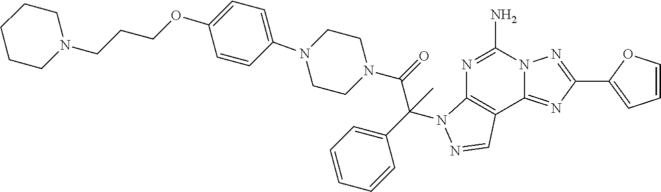 [SMILES: CC(C(=O)N1CCN(c2ccc(OCCCN3CCCCC3)cc2)CC1)(c1ccccc1)n1ncc2c1nc(N)n1nc(-c3ccco3)nc21]

To a stirred solution of 2-(5-amino-2-(furan-2-yl)-7H-pyrazolo[4,3-e][1,2,4]triazolo[1,5-c]pyrimidin-7-yl)-2-phenylpropanoic acid (70 mg, 0.18 mmol), HATU (75 mg, 0.20 mmol) and DIEA (70 mg, 0.54 mmol) in THF (10 ml) was added 1-(4-(3-(piperidin-1-yl)propoxy)phenyl)piperazine (55 mg, 0.18 mmol). The reaction mixture was stirred at rt for 15 h. After completion, the reaction mixture was diluted with EA (30 ml) and then washed with H₂O (15 ml×2). The organic layer was dried over Na₂SO₄, filtered and then concentrated under reduced pressure to afford a residue. The residue was purified by prep-TLC with DCM:MeOH (10:1) to afford the product (33.4 mg, 28%). ¹H NMR (400 MHz, DMSO-d6) δ 8.31 (s, 1H), 8.08 (s, 2H), 7.95 (s, 1H), 7.35-7.27 (m, 3H), 7.25 (d, J=4 Hz, 1H), 7.20 (d, J=8 Hz, 2H), 6.75-6.70 (m, 5H), 3.85 (t, J=4 Hz, 2H), 3.78-3.52 (m, 2H), 3.31-3.29 (m, 2H), 3.10-2.85 (m, 4H), 2.33 (s, 3H), 1.86-1.76 (m, 2H), 1.52-1.34 (m, 7H), 1.24-1.06 (m, 5H) ppm. MS: M/e 675 (M+1)⁺.

### Compound A86: 2-(5-amino-2-(furan-2-yl)-7H-pyrazolo[4,3-e][1,2,4]triazolo[1,5-c]pyrimidin-7-yl)-2-(3-fluorophenyl)-1-(4-(4-(2-methoxyethoxy)phenyl)piperazin-1-yl)propan-1-one

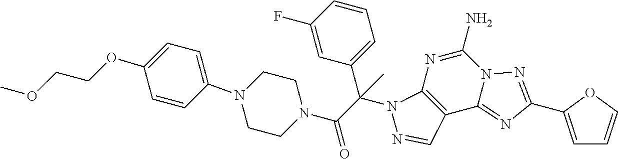 [SMILES: COCCOc1ccc(N2CCN(C(=O)C(C)(c3cccc(F)c3)n3ncc4c3nc(N)n3nc(-c5ccco5)nc43)CC2)cc1]

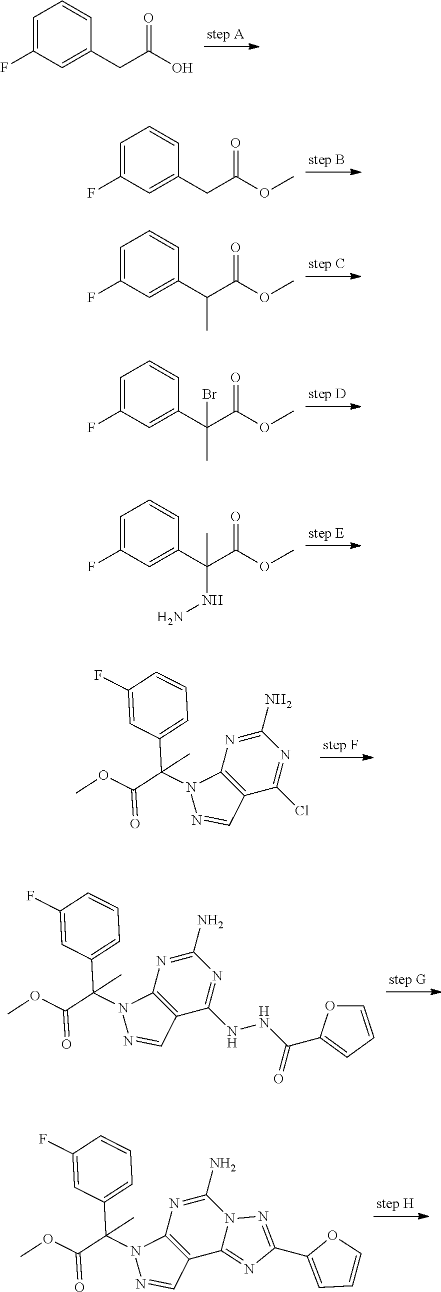

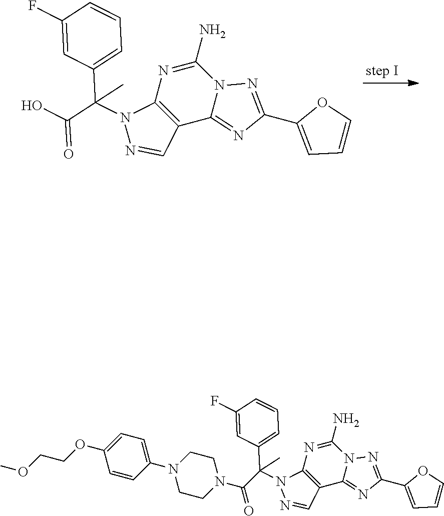 [SMILES: CC(C(=O)O)(c1cccc(F)c1)n1ncc2c1nc(N)n1nc(-c3ccco3)nc21 ; COCCOc1ccc(N2CCN(C(=O)C(C)(c3cccc(F)c3)n3ncc4c3nc(N)n3nc(-c5ccco5)nc43)CC2)cc1]

### Step A: methyl 2-(3-fluorophenyl)acetate

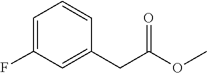

To a solution of 2-(3-fluorophenyl)acetic acid (5 g, 32.47 mmol) in MeOH (15 mL), sulfoxide chloride (5.80 g, 48.70 mmol) was added dropwise at 0° C., After the addition, the reaction mixture was stirred at rt for 3 h. The mixture was concentrated, quenched with ice water (20 mL), extracted with EtOAc (30 mL×3). The combined organic layers were washed with brine, dried over Na₂SO₄, concentrated and purified by column chromatography (petroleum ether/EtOAc=20:1˜5:1) to give methyl 2-(3-fluorophenyl)acetate (5.12 g, 93.87%) as yellow oil. MS: M/e 169 (M+1)⁺.

### Step B: methyl 2-(3-fluorophenyl)propanoate

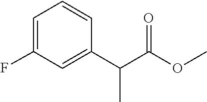

To a solution of LDA (44.64 mmol) in THF (20 mL) was added methyl 2-(3-fluorophenyl)acetate (5 g, 29.76 mmol) dropwise at −78° C. After the addition, the reaction mixture was warmed slowly to rt and stirred for 2 h. Then the reaction mixture was added with iodomethane (12.68 g, 89.29 mmol) at −40° C., After the addition, the reaction mixture was warmed slowly to rt and stirred for 2 h. The mixture was quenched with ice water (20 mL), extracted with EtOAc (30 mL×3). The combined organic layers were washed with brine, dried over Na₂SO₄, concentrated and purified by column chromatography (petroleum ether/EtOAc=20:1˜5:1) to give methyl 2-(3-fluorophenyl)propanoate (4.12 g, 76.07%) as yellow oil. ¹H NMR (400 MHz, CDCl₃) δ 7.30-7.25 (m, 1H), 7.07 (d, J=7.7 Hz, 1H), 7.02 (d, J=9.9 Hz, 1H), 6.95 (td, J=8.4, 1.7 Hz, 1H), 3.67 (s, 3H), 1.50 (d, J=7.2 Hz, 3H). MS: M/e 183 (M+1)⁺.

### Step C: methyl 2-bromo-2-(3-fluorophenyl)propanoate

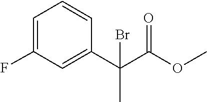

A mixture of methyl 2-(3-fluorophenyl)propanoate (4.12 g, 22.64 mmol), NBS (4.84 g, 27.16 mmol), BPO (0.274 g, 1.132 mmol) in carbon tetrachloride (20 mL) was stirred at 70° C. overnight. The mixture was concentrated, the residue was washed with PE and filtered, the filtrate was concentrated to give methyl 2-bromo-2-(3-fluorophenyl)propanoate (5.32 g, 90.38%) as yellow oil. ¹H NMR (400 MHz, CDCl3) δ 7.34-7.28 (m, 1H), 7.07 (d, J=7.7 Hz, 1H), 7.02 (d, J=9.3 Hz, 1H), 6.95 (td, J=8.4, 2.3 Hz, 1H), 3.81 (s, 3H), 2.29 (s, 3H).

### Step D: methyl 2-(3-fluorophenyl)-2-hydrazinylpropanoate

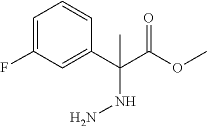

A mixture of methyl 2-bromo-2-(3-fluorophenyl)propanoate (5.32 g, 20.46 mmol), hydrazinium hydroxide (4.84 g, 81.85 mmol) in CH₃CN (20 mL) was stirred at 60° C. overnight. The mixture was extracted with EtOAc (30 mL×3). The combined organic layers were washed with water (10 mL×3) and brine, dried over Na₂SO₄, concentrated to give methyl 2-(3-fluorophenyl)-2-hydrazinylpropanoate (3.10 g, 71.46%) as brown oil. MS: M/e 213 (M+1)⁺.

### Step E: methyl 2-(6-amino-4-chloro-1H-pyrazolo[3,4-d]pyrimidin-1-yl)-2-(3-fluorophenyl)propanoate

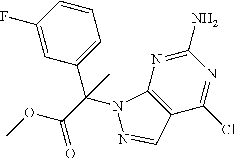 [SMILES: COC(=O)C(C)(c1cccc(F)c1)n1ncc2c(Cl)nc(N)nc21]

A mixture of methyl 2-(3-fluorophenyl)-2-hydrazinylpropanoate (3.10 g, 14.62 mmol), 2-amino-4,6-dichloropyrimidine-5-carbaldehyde (2.79 g, 14.62 mmol) in CH₃CN (20 mL) was stirred at rt overnight, then warmed to 70° C. and stirred for 2 h. The reaction mixture was poured into H₂O (20 mL) and extracted with EtOAc (20 mL×3). The combined organic layers were washed with brine, dried over Na₂SO₄, concentrated and purified by column chromatography (petroleum ether/EtOAc=10:1˜2:1) to give the product (4.03 g, 78.97%) as yellow solid. MS: M/e 350 (M+1)⁺.

### Step F: methyl 2-(6-amino-4-(2-(furan-2-carbonyl)hydrazinyl)-1H-pyrazolo[3,4-d]pyrimidin-1-yl)-2-(3-fluorophenyl)propanoate

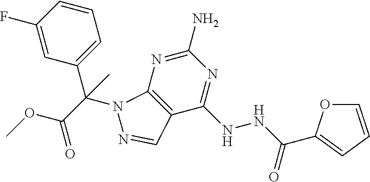 [SMILES: COC(=O)C(C)(c1cccc(F)c1)n1ncc2c(NNC(=O)c3ccco3)nc(N)nc21]

A mixture of methyl 2-(6-amino-4-chloro-1H-pyrazolo[3,4-d]pyrimidin-1-yl)-2-(3-fluorophenyl)propanoate (4.03 g, 11.55 mmol), furan-2-carbohydrazide (1.75 g, 13.86 mmol), DIPEA (2.98 g, 23.09 mmol) in DMSO (5 mL) was stirred at 120° C. overnight. The reaction mixture was poured into H₂O (10 mL) and extracted with EtOAc (20 mL×3). The combined organic layers were washed with brine, dried over Na₂SO₄, concentrated and purified by column chromatography (petroleum ether/EtOAc=10:1-1:1) to give the product (4.39 g, 86.60%) as yellow solid. MS: M/e 440 (M+1)⁺.

### Step G: methyl 2-(5-amino-2-(furan-2-yl)-7H-pyrazolo[4,3-e][1,2,4]triazolo[1,5-c]pyrimidin-7-yl)-2-(3-fluorophenyl)propanoate

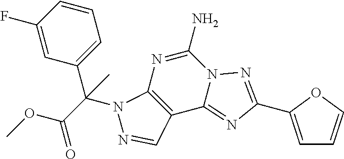 [SMILES: COC(=O)C(C)(c1cccc(F)c1)n1ncc2c1nc(N)n1nc(-c3ccco3)nc21]

A mixture of methyl 2-(6-amino-4-(2-(furan-2-carbonyl)hydrazinyl)-1H-pyrazolo[3,4-d]pyrimidin-1-yl)-2-(3-fluorophenyl)propanoate (4.39 g, 9.95 mmol), trimethylsilyl (E)-N-(trimethylsilyl)acetimidate (3 mL), hexamethyldisilazane (HMDS, 3 mL) was stirred at 120° C. overnight. The reaction mixture was concentrated, stirred in MeOH (10 mL) and water (2 mL) at 50° C. for 1 h. Then concentrated and purified by column chromatography (petroleum ether/EtOAc=10:1˜1:1) to give the product (3.75 g, 89.5%) as white solid. MS: M/e 422 (M+1)⁺.

### Step H: 2-(5-amino-2-(furan-2-yl)-7H-pyrazolo[4,3-e][1,2,4]triazolo[1,5-c]pyrimidin-7-yl)-2-(3-fluorophenyl)propanoic Acid

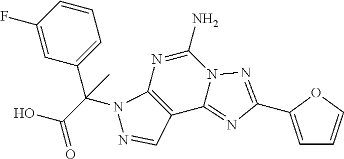 [SMILES: CC(C(=O)O)(c1cccc(F)c1)n1ncc2c1nc(N)n1nc(-c3ccco3)nc21]

A mixture of methyl 2-(5-amino-2-(furan-2-yl)-7H-pyrazolo[4,3-e][1,2,4]triazolo[1,5-c]pyrimidin-7-yl)-2-(3-fluorophenyl)propanoate (3.75 g, 8.91 mmol), lithium hydroxide (3.74 g, 89.07 mmol) in MeOH (5 mL) and water (2 mL) was stirred at 50° C. overnight. The reaction mixture was acidified with hydrochloric acid, extracted with EtOAc (20 mL×3). The combined organic layers were washed with brine, dried over Na₂SO₄, concentrated and purified by column chromatography (petroleum ether/EtOAc=10:1˜1:2) to give the product (3.45 g, 95.16%) as white solid. MS: M/e 408 (M+1)⁺.

### Step G: 2-(5-amino-2-(furan-2-yl)-7H-pyrazolo[4,3-e][1,2,4]triazolo[1,5-c]pyrimidin-7-yl)-2-(3-fluorophenyl)-1-(4-(4-(2-methoxyethoxy)phenyl)piperazin-1-yl)propan-1-one

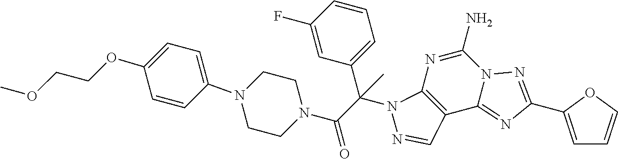 [SMILES: COCCOc1ccc(N2CCN(C(=O)C(C)(c3cccc(F)c3)n3ncc4c3nc(N)n3nc(-c5ccco5)nc43)CC2)cc1]

A mixture of 2-(5-amino-2-(furan-2-yl)-7H-pyrazolo[4,3-e][1,2,4]triazolo[1,5-c]pyrimidin-7-yl)-2-(3-fluorophenyl)propanoic acid (3.45 g, 8.48 mmol), 1-(4-(2-methoxyethoxy)phenyl)piperazine (2.40 g, 10.17 mmol), HATU (4.83 g, 12.71 mmol), DIPEA (3.28 g, 25.43 mmol) in DMF (10 mL) was stirred at rt overnight. The reaction mixture was poured into H₂O (10 mL) and extracted with EtOAc (15 mL×3). The combined organic layers were washed with brine, dried over Na₂SO₄, concentrated and purified by column chromatography (DCM/MeOH=40:1˜10:1) to give the product (3.75 g, 70.78%) as white solid. ¹H NMR (400 MHz, DMSO-d6) δ 8.36 (s, 1H), 8.13 (s, 2H), 7.97-7.94 (m, 1H), 7.33 (dd, J=14.6, 8.2 Hz, 1H), 7.24 (dd, J=3.4, 0.7 Hz, 1H), 7.12 (td, J=8.3, 2.1 Hz, 1H), 7.05 (d, J=11.1 Hz, 1H), 6.88 (d, J=8.2 Hz, 1H), 6.84 (s, 1H), 6.80 (d, J=7.9 Hz, 3H), 6.74 (dd, J=3.4, 1.8 Hz, 1H), 4.00-3.94 (m, 3H), 3.88-3.64 (m, 2H), 3.62-3.56 (m, 3H), 3.27 (s, 3H), 3.18-2.65 (m, 6H), 2.38 (s, 3H). MS: M/e 626 (M+1)+.

### Compound A87: 2-(5-amino-2-(furan-2-yl)-7H-pyrazolo[4,3-e][1,2,4]triazolo[1,5-c]pyrimidin-7-yl)-2-(2-fluorophenyl)-1-(4-(4-(2-methoxyethoxy)phenyl)piperazin-1-yl)propan-1-one

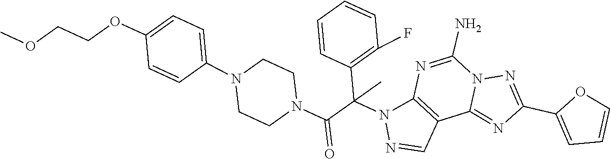 [SMILES: COCCOc1ccc(N2CCN(C(=O)C(C)(c3ccccc3F)n3ncc4c3nc(N)n3nc(-c5ccco5)nc43)CC2)cc1]

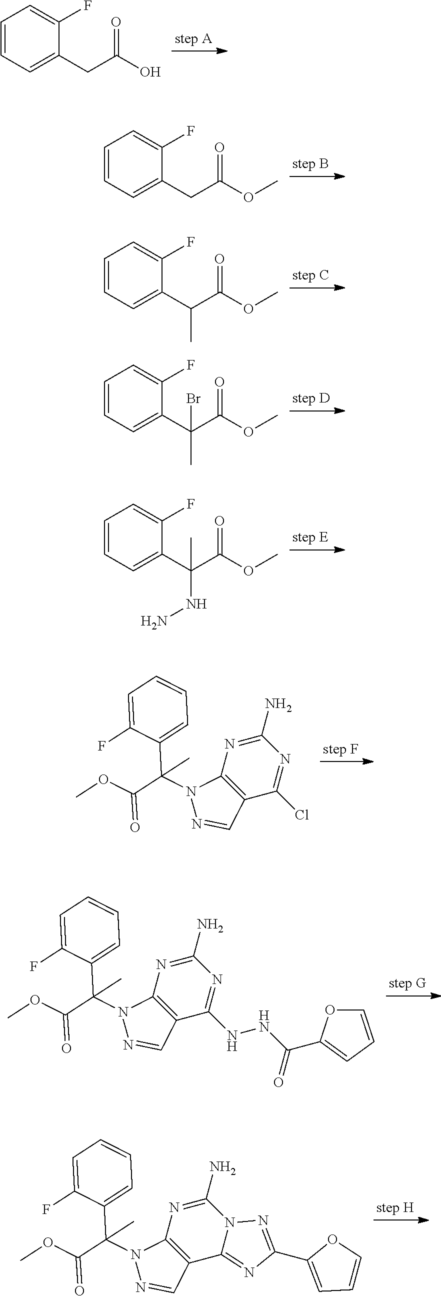

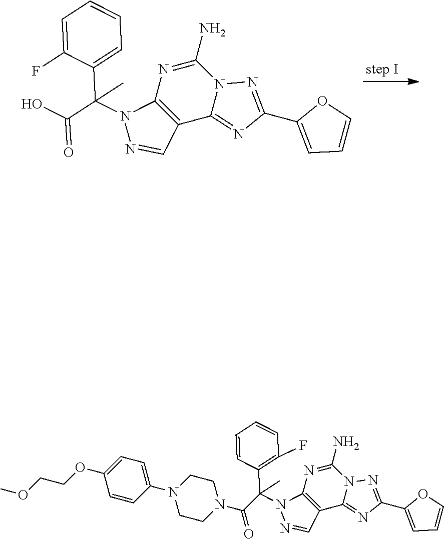 [SMILES: CC(C(=O)O)(c1ccccc1F)n1ncc2c1nc(N)n1nc(-c3ccco3)nc21 ; COCCOc1ccc(N2CCN(C(=O)C(C)(c3ccccc3F)n3ncc4c3nc(N)n3nc(-c5ccco5)nc43)CC2)cc1]

### Step A: methyl 2-(2-fluorophenyl)acetate

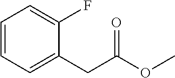

To a solution of 2-(2-fluorophenyl)acetic acid (5 g, 32.47 mmol) in MeOH (15 mL), sulfoxide chloride (5.80 g, 48.70 mmol) was added dropwise at 0° C., After the addition, the reaction mixture was stirred at rt for 3 h. The mixture was concentrated, quenched with ice water (20 mL), extracted with EtOAc (30 mL×3). The combined organic layers were washed with brine, dried over Na₂SO₄, concentrated and purified by column chromatography (petroleum ether/EtOAc=20:1˜5:1) to give methyl 2-(2-fluorophenyl)acetate (5.23 g, 95.88%) as yellow oil. MS: M/e 169 (M+1)⁺.

### Step B: methyl 2-(2-fluorophenyl)propanoate

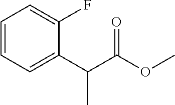

To a solution of LDA (44.64 mmol) in THF (20 mL) was added methyl 2-(2-fluorophenyl)acetate (5 g, 29.76 mmol) dropwise at −78° C. After the addition, the reaction mixture was warmed slowly to rt and stirred for 2 h. Then the reaction mixture was added with iodomethane (12.68 g, 89.29 mmol) at −40° C. After the addition, the reaction mixture was warmed slowly to rt and stirred for 2 h. The mixture was quenched with ice water (20 mL), extracted with EtOAc (30 mL×3). The combined organic layers were washed with brine, dried over Na₂SO₄, concentrated and purified by column chromatography (petroleum ether/EtOAc=20:1˜5:1) to give methyl 2-(2-fluorophenyl)propanoate (4.33 g, 79.94%) as yellow oil. MS: M/e 183 (M+1)⁺.

### Step C: methyl 2-bromo-2-(2-fluorophenyl)propanoate

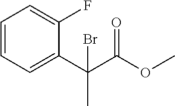

A mixture of methyl 2-(2-fluorophenyl)propanoate (4.33 g, 23.79 mmol), NBS (5.08 g, 28.55 mmol), BPO (0.288 g, 1.190 mmol) in carbon tetrachloride (20 mL) was stirred at 70° C. overnight. The mixture was concentrated, the residue was washed with PE and filtered, the filtrate was concentrated to give methyl 2-bromo-2-(2-fluorophenyl)propanoate (5.66 g, 91.50%) as yellow oil. ¹H NMR (400 MHz, CDCl₃) δ 7.76 (td, J=8.1, 1.6 Hz, 1H), 7.37-7.30 (m, 1H), 7.19 (td, J=7.7, 1.1 Hz, 1H), 7.03 (ddd, J=11.4, 8.2, 1.1 Hz, 1H), 3.80 (s, 3H), 2.25 (s, 3H).

### Step D: methyl 2-(2-fluorophenyl)-2-hydrazinylpropanoate

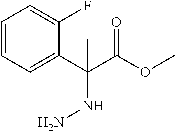

A mixture of methyl 2-bromo-2-(2-fluorophenyl)propanoate (5.66 g, 21.77 mmol), hydrazinium hydroxide (5.14 g, 87.08 mmol) in CH₃CN (20 mL) was stirred at 60° C. overnight. The mixture was extracted with EtOAc (30 mL×3). The combined organic layers were washed with water (10 mL×3) and brine, dried over Na₂SO₄, concentrated to give methyl 2-(2-fluorophenyl)-2-hydrazinylpropanoate (2.95 g, 63.92%) as brown oil. MS: M/e 213 (M+1)⁺.

### Step E: methyl 2-(6-amino-4-chloro-1H-pyrazolo[3,4-d]pyrimidin-1-yl)-2-(2-fluorophenyl)propanoate

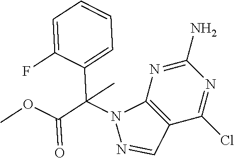 [SMILES: COC(=O)C(C)(c1ccccc1F)n1ncc2c(Cl)nc(N)nc21]

A mixture of methyl 2-(2-fluorophenyl)-2-hydrazinylpropanoate (2.95 g, 13.92 mmol), 2-amino-4,6-dichloropyrimidine-5-carbaldehyde (2.69 g, 13.91 mmol) in CH₃CN (20 mL) was stirred at rt overnight, then warmed to 70° C. and stirred for 2 h. The reaction mixture was poured into H₂O (20 mL) and extracted with EtOAc (20 mL×3). The combined organic layers were washed with brine, dried over Na₂SO₄, concentrated and purified by column chromatography (petroleum ether/EtOAc=10:1˜2:1) to give methyl 2-(6-amino-4-chloro-1H-pyrazolo[3,4-d]pyrimidin-1-yl)-2-(2-fluorophenyl)propanoate (3.56 g, 73.33%) as yellow solid. MS: M/e 350 (M+1)⁺.

### Step F: methyl 2-(6-amino-4-(2-(furan-2-carbonyl)hydrazinyl)-1H-pyrazolo[3,4-d]pyrimidin-1-yl)-2-(2-fluorophenyl)propanoate

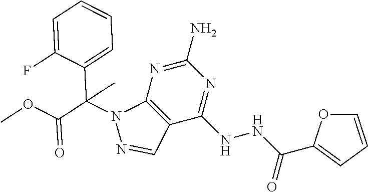 [SMILES: COC(=O)C(C)(c1ccccc1F)n1ncc2c(NNC(=O)c3ccco3)nc(N)nc21]

A mixture of methyl 2-(6-amino-4-chloro-1H-pyrazolo[3,4-d]pyrimidin-1-yl)-2-(2-fluorophenyl)propanoate (3.56 g, 10.20 mmol), furan-2-carbohydrazide (1.54 g, 12.24 mmol), DIPEA (2.63 g, 20.28 mmol) in DMSO (5 mL) was stirred at 120° C. overnight. The reaction mixture was poured into H₂O (10 mL) and extracted with EtOAc (20 mL×3). The combined organic layers were washed with brine, dried over Na₂SO₄, concentrated and purified by column chromatography (petroleum ether/EtOAc=10:1˜1:1) to give methyl 2-(6-amino-4-(2-(furan-2-carbonyl)hydrazinyl)-1H-pyrazolo[3,4-d]pyrimidin-1-yl)-2-(2-fluorophenyl)propanoate (3.95 g, 88.21%) as yellow solid. MS: M/e 440 (M+1)⁺.

### Step G: methyl 2-(5-amino-2-(furan-2-yl)-7H-pyrazolo[4,3-e][1,2,4]triazolo[1,5-c]pyrimidin-7-yl)-2-(2-fluorophenyl)propanoate

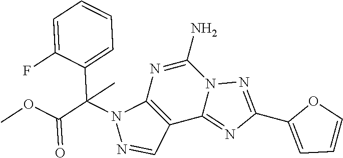 [SMILES: COC(=O)C(C)(c1ccccc1F)n1ncc2c1nc(N)n1nc(-c3ccco3)nc21]

A mixture of methyl 2-(6-amino-4-(2-(furan-2-carbonyl)hydrazinyl)-1H-pyrazolo[3,4-d]pyrimidin-1-yl)-2-(2-fluorophenyl)propanoate (3.95 g, 8.997 mmol), trimethylsilyl (E)-N-(trimethylsilyl)acetimidate (3 mL), HMDS (3 mL) was stirred at 120° C. overnight. The reaction mixture was concentrated, stirred in MeOH (10 mL) and water (2 mL) at 50° C. for 1 h. Then concentrated and purified by column chromatography (petroleum ether/EtOAc=10:1˜1:1) to give methyl 2-(5-amino-2-(furan-2-yl)-7H-pyrazolo[4,3-e][1,2,4]triazolo[1,5-c]pyrimidin-7-yl)-2-(2-fluorophenyl)propanoate (3.30 g, 87.12%) as white solid. MS: M/e 422 (M+1)⁺.

### Step H: 2-(5-amino-2-(furan-2-yl)-7H-pyrazolo[4,3-e][1,2,4]triazolo[1,5-c]pyrimidin-7-yl)-2-(2-fluorophenyl)propanoic Acid

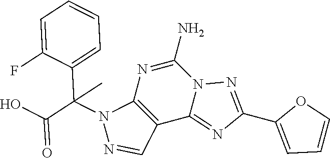 [SMILES: CC(C(=O)O)(c1ccccc1F)n1ncc2c1nc(N)n1nc(-c3ccco3)nc21]

A mixture of methyl 2-(5-amino-2-(furan-2-yl)-7H-pyrazolo[4,3-e][1,2,4]triazolo[1,5-c]pyrimidin-7-yl)-2-(2-fluorophenyl)propanoate (3.30 g, 7.838 mmol), lithium hydroxide (3.14 g, 78.38 mmol) in MeOH (5 mL) and water (2 mL) was stirred at 50° C. overnight. The reaction mixture was acidified with hydrochloric acid, extracted with EtOAc (20 mL×3). The combined organic layers were washed with brine, dried over Na₂SO₄, concentrated and purified by column chromatography (petroleum ether/EtOAc=10:1˜1:2) to give 2-(5-amino-2-(furan-2-yl)-7H-pyrazolo[4,3-e][1,2,4]triazolo[1,5-c]pyrimidin-7-yl)-2-(2-fluorophenyl)propanoic acid (2.87 g, 89.74%) as white solid. MS: M/e 408 (M+1)⁺.

### Step G: 2-(5-amino-2-(furan-2-yl)-7H-pyrazolo[4,3-e][1,2,4]triazolo[1,5-c]pyrimidin-7-yl)-2-(2-fluorophenyl)-1-(4-(4-(2-methoxyethoxy)phenyl)piperazin-1-yl)propan-1-one

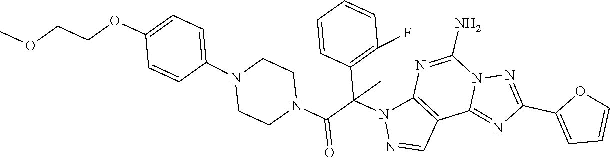 [SMILES: COCCOc1ccc(N2CCN(C(=O)C(C)(c3ccccc3F)n3ncc4c3nc(N)n3nc(-c5ccco5)nc43)CC2)cc1]

A mixture of 2-(5-amino-2-(furan-2-yl)-7H-pyrazolo[4,3-e][1,2,4]triazolo[1,5-c]pyrimidin-7-yl)-2-(2-fluorophenyl)propanoic acid (2.87 g, 7.052 mmol), 1-(4-(2-methoxyethoxy)phenyl)piperazine (2.00 g, 8.462 mmol), HATU (4.02 g, 10.58 mmol), DIPEA (2.73 g, 21.15 mmol) in DMF (10 mL) was stirred at rt overnight. The reaction mixture was poured into H₂O (10 mL) and extracted with EtOAc (15 mL×3). The combined organic layers were washed with brine, dried over Na₂SO₄, concentrated and purified by column chromatography (DCM/MeOH=40:1˜10:1) to give the product (3.45 g, 78.16%) as white solid. ¹H NMR (400 MHz, DMSO-d6) δ 8.31 (s, 1H), 8.09 (s, 2H), 7.95 (s, 1H), 7.39 (dd, J=12.6, 7.0 Hz, 1H), 7.25 (q, J=8.4 Hz, 2H), 7.10 (t, J=7.5 Hz, 1H), 6.78-6.66 (m, 6H), 3.96 (m, 2H), 3.72 (s, 8H), 3.58 (m, 2H), 3.27 (s, 3H), 2.44 (s, 3H). MS: M/e 626 (M+1)⁺.

### Compound A88: 3-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)-N,N-dimethylbenzamide

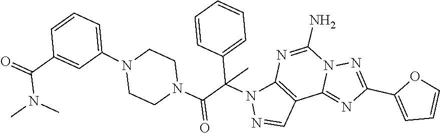 [SMILES: CN(C)C(=O)c1cccc(N2CCN(C(=O)C(C)(c3ccccc3)n3ncc4c3nc(N)n3nc(-c5ccco5)nc43)CC2)c1]

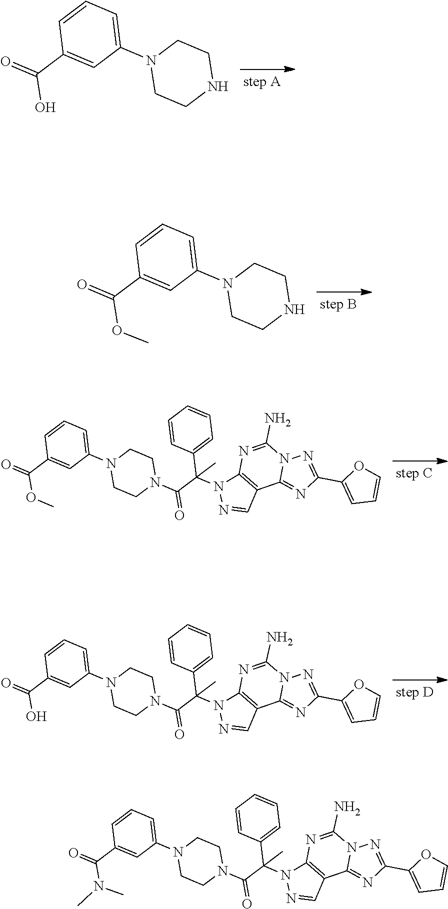 [SMILES: O=C(O)c1cccc(N2CCNCC2)c1 ; COC(=O)c1cccc(N2CCNCC2)c1 ; COC(=O)c1cccc(N2CCN(C(=O)C(C)(c3ccccc3)n3ncc4c3nc(N)n3nc(-c5ccco5)nc43)CC2)c1 ; CC(C(=O)N1CCN(c2cccc(C(=O)O)c2)CC1)(c1ccccc1)n1ncc2c1nc(N)n1nc(-c3ccco3)nc21 ; CN(C)C(=O)c1cccc(N2CCN(C(=O)C(C)(c3ccccc3)n3ncc4c3nc(N)n3nc(-c5ccco5)nc43)CC2)c1]

### Step A: methyl 3-(piperazin-1-yl)benzoate

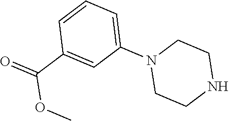 [SMILES: COC(=O)c1cccc(N2CCNCC2)c1]

To a stirred solution of 3-(piperazin-1-yl)benzoic acid (1 g, 4.85 mmol) in MeOH (10 mL) was added SOCl₂ (1.15 g, 9.76 mmol) dropwise. After the addition, the reaction mixture was stirred at 60° C. overnight. Most solvent was removed to give the residue, which was treated with EtOAc/aq.K₂CO₃ and extracted with EtOAc (30 mL×3). The combined organic layers were washed with brine, dried over Na₂SO₄, concentrated to give the target compound (0.5 g, 46.8%) as colorless oil. MS: M/e 221 (M+1)⁺.

### Step B: methyl 3-(4-(2-(5-amino-2-(furan-2-yl)-7H-pyrazolo[4,3-e][1,2,4]triazolo[1,5-c]pyrimidin-7-yl)-2-phenylpropanol)piperazin-1-yl)benzoate

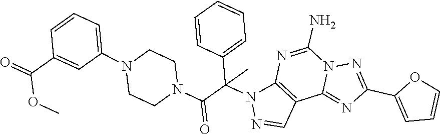 [SMILES: COC(=O)c1cccc(N2CCN(C(=O)C(C)(c3ccccc3)n3ncc4c3nc(N)n3nc(-c5ccco5)nc43)CC2)c1]

A mixture of 2-(5-amino-2-(furan-2-yl)-7H-pyrazolo[4,3-e][1,2,4]triazolo[1,5-c]pyrimidin-7-yl)-2-phenylpropanoic acid (300 mg, 0.77 mmol), the product of step A (170 mg, 0.77 mmol), HATU (353 mg, 0.92 mmol) and DIPEA (199 mg, 1.54 mmol) in DMF (4 mL) was stirred overnight. The reaction mixture was poured into H₂O (15 mL) and extracted with brine, dried over Na₂SO₄, concentrated and purified by column chromatography (petroleum ether/EtOAc=4:1˜1:1) to give the target compound (412 mg, 90.5%) as a white solid. MS: M/e 592 (M+1)⁺.

### Step C: 3-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)benzoic Acid

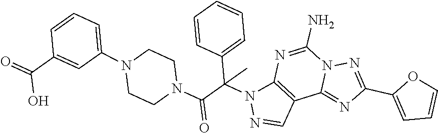 [SMILES: CC(C(=O)N1CCN(c2cccc(C(=O)O)c2)CC1)(c1ccccc1)n1ncc2c1nc(N)n1nc(-c3ccco3)nc21]

To a stirred solution of the product of step C (410 mg, 0.69 mmol) in MeOH (10 mL) was added aq.NaOH (2.0 M, 10 mL). After the addition, the reaction was stirred overnight. Most of MeOH was removed to give the aqueous layer, which was acidified to pH=3˜4 with aq.HCl and extracted with EtOAc (30 mL×3). The combined organic layers were washed with brine, dried over Na₂SO₄ and concentrated to give the target compound (350 mg, 87.9%) as a white solid. MS: M/e 578 (M+1)⁺.

### Step D: 3-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)-N,N-dimethylbenzamide

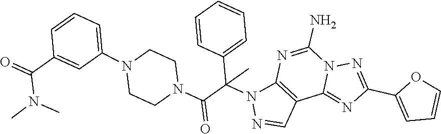 [SMILES: CN(C)C(=O)c1cccc(N2CCN(C(=O)C(C)(c3ccccc3)n3ncc4c3nc(N)n3nc(-c5ccco5)nc43)CC2)c1]

A mixture of the product of step C (50 mg, 0.087 mmol), dimethylamine hydrochloride (7.1 mg, 0.087 mmol), HATU (40 mg, 0.104 mmol) and DIPEA (22.4 mg, 0.174 mmol) in DMF (4 mL) was stirred overnight. The reaction mixture was poured into H₂O (10 mL), then extracted with EtOAc (15 mL×3). The combined organic layers were washed with brine, dried over Na₂SO₄, concentrated and purified by prep-TLC (CH₂Cl₂/MeOH=10:1) to give the target compound (21 mg, 40%) as a white solid. ¹H NMR (400 MHz, DMSO-d6) δ 8.32 (s, 1H), 8.07 (s, 2H), 7.95 (s, 1H), 7.38-7.14 (m, 7H), 6.85 (d, J=8.4 Hz, 1H), 6.77-6.68 (m, 3H), 3.89-3.56 (m, 2H), 325-2.95 (m, 4H), 2.91 (s, 3H), 2.80 (s, 3H), 2.76-2.56 (m, 2H), 2.34 (s, 3H) ppm. MS: M/e 605 (M+1)⁺.

### Compound A89: 2-(5-amino-2-(furan-2-yl)-7H-pyrazolo[4,3-e][1,2,4]triazolo[1,5-c]pyrimidin-7-yl)-2-phenyl-1-(4-(3-(piperidine-1-carbonyl)phenyl)piperazin-1-yl)propan-1-one

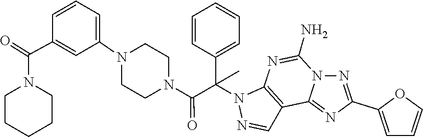 [SMILES: CC(C(=O)N1CCN(c2cccc(C(=O)N3CCCCC3)c2)CC1)(c1ccccc1)n1ncc2c1nc(N)n1nc(-c3ccco3)nc21]

A mixture of 3-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)benzoic acid (50 mg, 0.087 mmol), piperidine (7.4 mg, 0.087 mmol), HATU (40 mg, 0.104 mmol) and DIPEA (22.4 mg, 0.174 mmol) in DMF (4 mL) was stirred overnight. The reaction mixture was poured into H₂O (10 mL), then extracted with EtOAc (15 mL×3). The combined organic layers were washed with brine, dried over Na₂SO₄, concentrated and purified by prep-TLC (PE/EA=1:2) to give the target compound (27 mg, 48.1%). ¹H NMR (400 MHz, DMSO-d6) δ 8.32 (s, 1H), 8.07 (s, 2H), 7.95 (s, 1H), 7.40-7.13 (m, 7H), 6.84 (d, J=8.4 Hz, 1H), 6.75-6.72 (m, 1H), 6.70-6.63 (m, 2H), 3.86-3.42 (m, 4H), 3.26-2.86 (m, 6H), 2.34 (s, 3H), 1.64-1.21 (m, 8H) ppm. MS: M/e 645 (M+1)⁺.

### Compound A90: 2-(5-amino-2-(furan-2-yl)-7H-pyrazolo[4,3-e][1,2,4]triazolo[1,5-c]pyrimidin-7-yl)-2-phenyl-1-(4-(3-(cyclohexanaminocarbonyl)phenyl)piperazin-1-yl)propan-1-one

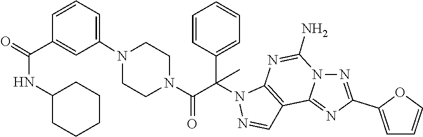 [SMILES: CC(C(=O)N1CCN(c2cccc(C(=O)NC3CCCCC3)c2)CC1)(c1ccccc1)n1ncc2c1nc(N)n1nc(-c3ccco3)nc21]

A mixture of 3-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)benzoic acid (50 mg, 0.087 mmol), cyclohexanamine (8.6 mg, 0.087 mmol), HATU (40 mg, 0.104 mmol) and DIPEA (22.4 mg, 0.174 mmol) in DMF (4 mL) was stirred overnight. The reaction mixture was poured into H₂O (10 mL), then extracted with EtOAc (15 mL×3). The combined organic layers were washed with brine, dried over Na₂SO₄, concentrated and purified by Pre-TLC (EA) to give the target compound (28 mg, 48.8%). ¹H NMR (400 MHz, DMSO-d6) δ 8.32 (s, 1H), 8.08 (s, 2H), 8.01 (d, J=8.0 Hz, 1H), 7.95 (s, 1H), 7.40-7.15 (m, 9H), 6.95-6.86 (m, 1H), 6.77-6.71 (m, 1H), 3.80-3.62 (mz, 2H), 3.26-2.92 (m, 4H), 2.85-2.59 (m, 2H), 2.35 (s, 3H), 1.75 (m, 4H), 1.66-1.53 (m, 1H), 1.38-1.18 (m, 5H), 1.19-1.03 (m, 1H) ppm. MS: M/e 659 (M+1)⁺.

### Compound A91: 3-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)-N-((3-hydroxyoxetan-3-yl)methyl)benzamide

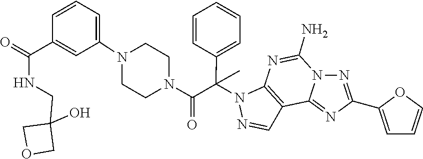 [SMILES: CC(C(=O)N1CCN(c2cccc(C(=O)NCC3(O)COC3)c2)CC1)(c1ccccc1)n1ncc2c1nc(N)n1nc(-c3ccco3)nc21]

A mixture of 3-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)benzoic acid (50 mg, 0.087 mmol), 3-(aminomethyl)oxetan-3-ol (8.9 mg, 0.087 mmol), HATU (40 mg, 0.104 mmol) and DIPEA (22.4 mg, 0.174 mmol) in DMF (4 mL) was stirred overnight. The reaction mixture was poured into H₂O (10 mL), then extracted with EtOAc (15 mL×3). The combined organic layers were washed with brine, dried over Na₂SO₄, concentrated and purified by prep-TLC (CH₂Cl₂/MeOH=10:1) to give the target compound (27 mg, 46.9%). ¹H NMR (400 MHz, DMSO-d6) δ 8.47 (t, J=6.0 Hz, 1H), 8.32 (s, 1H), 8.08 (s, 2H), 7.95 (s, 1H), 7.39-7.17 (m, 9H), 6.97-6.92 (m, 1H), 6.76-6.71 (m, 1H), 5.83 (s, 1H), 4.45 (d, J=6.4 Hz, 2H), 4.36 (d, J=6.4 Hz, 2H), 3.86-3.58 (m, 2H), 3.51 (d, J=6.0 Hz, 2H), 3.26-2.86 (m, 4H), 2.85-2.62 (m, 2H), 2.35 (s, 3H) ppm. MS: M/e 663 (M+1)⁺.

### Compound A92: 4-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)-N-methylbenzamide

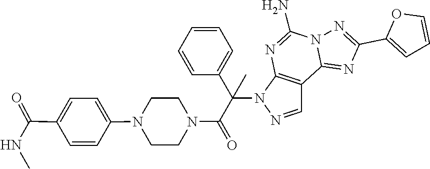 [SMILES: CNC(=O)c1ccc(N2CCN(C(=O)C(C)(c3ccccc3)n3ncc4c3nc(N)n3nc(-c5ccco5)nc43)CC2)cc1]

A mixture of 4-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)benzoic acid (40 mg, 0.07 mmol), methanamine hydrochloride (20 mg, 0.30 mmol), HATU (40 mg, 0.1 mmol) and DIEA (30 mg, 0.23 mmol) in DMF (5 mL) was stirred overnight at RT. The reaction mixture was poured into H₂O (20 mL) and extracted with EtOAc (50 mL×3). The combined organic layers were washed with brine, dried over Na₂SO₄, concentrated and purified by column chromatography (petroleum ether/EtOAc=1:1˜100% EtOAc) to give the target compound (8.5 mg, 20.5%) as a yellow solid. ¹H NMR (400 MHz, DMSO-d6) δ 8.32 (s, 1H), 8.09 (br, 3H), 7.95 (d, J=0.9 Hz, 1H), 7.63 (d, J=8.9 Hz, 2H), 7.38-7.15 (m, 6H), 6.80 (d, J=8.9 Hz, 2H), 6.74 (dd, J=3.4, 1.8 Hz, 1H), 3.87-3.54 (m, 2H), 3.28-3.13 (m, 3H), 3.10-2.92 (m, 2H), 2.71 (d, J=4.4 Hz, 4H), 2.34 (s, 3H) ppm. MS: M/e 591 (M+1)⁺.

### Compound A93: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(2-methoxyphenyl)propan-1-one

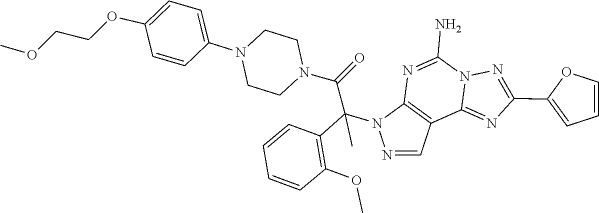 [SMILES: COCCOc1ccc(N2CCN(C(=O)C(C)(c3ccccc3OC)n3ncc4c3nc(N)n3nc(-c5ccco5)nc43)CC2)cc1]

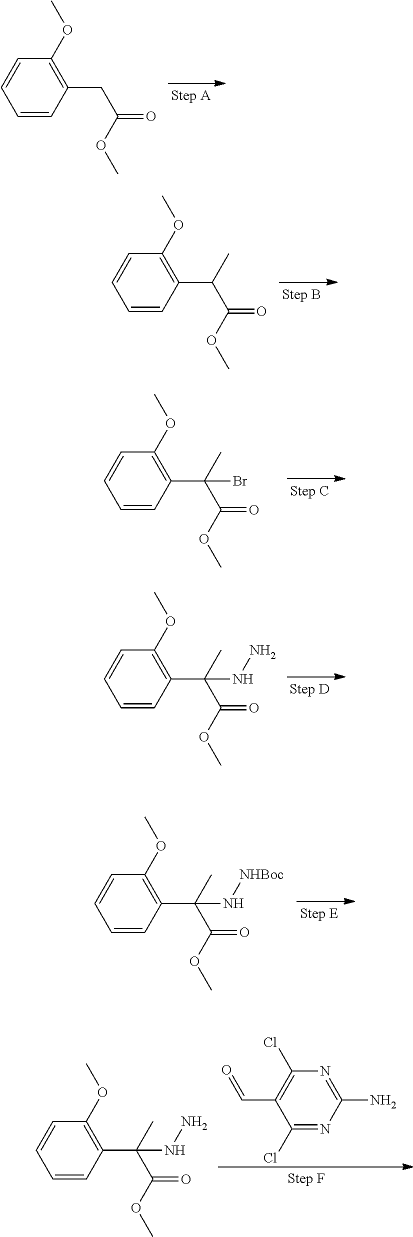

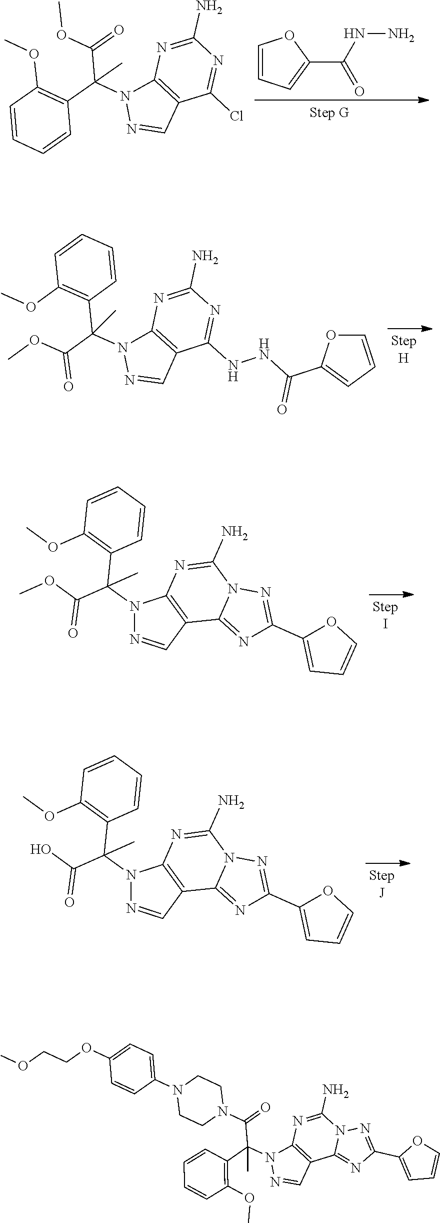

### Step A: methyl 2-(2-methoxyphenyl)propanoate

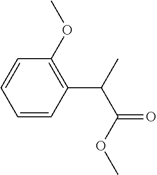

To a stirred solution of methyl 2-(2-methoxyphenyl)acetate (5.0 g, 27.7 mmol) in 60 mL of THF was added LDA (15 mL, 2.0 M) at −78° C. under N₂. The resulting mixture was stirred for 30 min. Mel (12.0 g, 84.5 mmol) was added in drops. The reaction mixture was allowed to warm to rt and stirred for 5 hrs. The mixture was quenched with 50 mL of H₂O, extracted with EA (50 mL×3). The organic extracts were combined, washed with brine (50 mL×3), dried over Na₂SO₄ and concentrated. The residue was purified by column chromatography to give the title product (4.9 g, 91%) as a light yellow oil. ¹H NMR (400 MHz, CDCl₃) δ 7.27-7.16 (m, 2H), 6.97-6.91 (m, 1H), 6.87 (d, J=8.0 Hz, 1H), 4.05 (q, J=7.2 Hz, 1H), 3.82 (s, 3H), 3.65 (s, 3H), 1.45 (d, J=7.2 Hz, 3H).

### Step B: methyl 2-bromo-2-(2-methoxyphenyl)propanoate

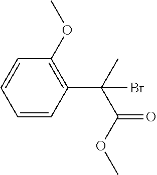

To a stirred solution of methyl 2-(2-methoxyphenyl)propanoate (2.9 g, 14.9 mmol) in 30 mL of THF was added LDA (8.5 mL, 2.0 M) at −78° C. under N₂. The resulting mixture was stirred for 30 min. TMSCl (1.8 g, 16.5 mmol) was added and the resulting mixture was stirred at rt for 2 hrs. The mixture was added with NBS (4.2 g, 23.5 mmol) at −78° C. The resulting mixture was stirred at −78° C. for 1 hour. Then the reaction mixture was allowed to warm to rt and stirred for another 1 hour. The mixture was quenched with saturated aqueous solution of NaHCO₃ (30 mL), extracted with EA (50 mL×2). The combined extracts were washed with brine (50 mL×2), dried over Na₂SO₄ and concentrated to give the title product (3.3 g, crude) as a light brown oil which was used for the next step directly. ¹H NMR (400 MHz, CDCl₃) δ 7.76 (dd, J=7.6, 1.2 Hz, 1H), 7.37-7.30 (m, 1H), 7.01 (t, J=7.6 Hz, 1H), 6.86 (d, J=8.0 Hz, 1H), 3.81 (s, 3H), 3.74 (s, 3H), 2.21 (s, 3H).

### Step C: methyl 2-hydrazinyl-2-(2-methoxyphenyl)propanoate

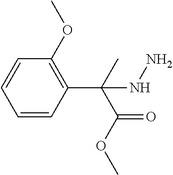 [SMILES: COC(=O)C(C)(NN)c1ccccc1OC]

To a stirred solution of methyl 2-bromo-2-(2-methoxyphenyl)propanoate (3.3 g, 12.1 mmol) in MeCN (20 mL) was added hydrazine hydrate (5 mL, 100 mmol) at rt and the resulting mixture was stirred at 50° C. for 16 hrs. 20 mL of EA was added and the mixture was washed with brine (20 mL×3), dried over Na₂SO₄, and concentrated to dryness to give the title product (2.9 g, crude) as a brown oil. MS: M/e 225 (M+1)⁺.

### Step D: tert-butyl 2-(1-methoxy-2-(2-methoxyphenyl)-1-oxopropan-2-yl)hydrazine-1-carboxylate

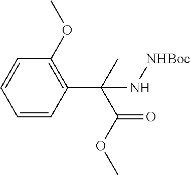 [SMILES: COC(=O)C(C)(NNC(=O)OC(C)(C)C)c1ccccc1OC]

To a mixture of methyl 2-hydrazinyl-2-(2-methoxyphenyl)propanoate (280 mg, 1.0 mmol) and Et₃N (5.0 g, 50 mmol) in CH₂Cl₂ (20 mL) was added Boc₂O (3.3 g, 15.1 mmol) in drops at 0° C. and the resulting mixture was stirred at rt for 16 hrs. The mixture was concentrated, diluted with 30 mL of EA and washed with brine (20 mL×3), dried over Na₂SO₄, and concentrated. The residue was purified by column chromatography to give the title product (660 mg, 8% for 3 steps) as a light brown oil. MS: M/e 325 (M+1)⁺.

### Step E: methyl 2-hydrazinyl-2-(2-methoxyphenyl)propanoate hydrochloride

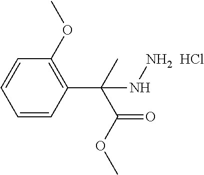

To a stirred solution of tert-butyl 2-(1-methoxy-2-(2-methoxyphenyl)-1-oxopropan-2-yl)hydrazine-1-carboxylate (660 mg, 2.04 mmol) in EA (10 mL) was added HCl/EA (3 mL, 4M), and the resulting mixture was stirred at rt for 24 hrs. The mixture was concentrated to dryness, 10 mL of EA was added and the resulting suspension was concentrated again to remove the excess of HCl to give the title product (520 mg, crude) as a gray solid. MS: M/e 225 (M+1)⁺.

### Step F: methyl 2-(6-amino-4-chloro-1H-pyrazolo[3,4-d]pyrimidin-1-yl)-2-(2-methoxyphenyl)propanoate

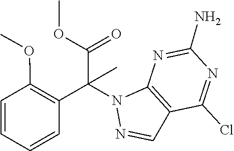 [SMILES: COC(=O)C(C)(c1ccccc1OC)n1ncc2c(Cl)nc(N)nc21]

A mixture of methyl 2-hydrazinyl-2-(2-methoxyphenyl)propanoate hydrochloride (510 mg, 2.0 mmol) and 2-amino-4,6-dichloropyrimidine-5-carbaldehyde (385 mg, 2.0 mmol) in MeCN (14 mL) was stirred at rt for 16 hrs. The resulting mixture was then heated at 70° C. for 1 hour. Cooled to rt. 1.0 mL of DIEA was added. The mixture was concentrated and diluted with 20 mL of EA, washed with brine (10 mL×3), dried over Na₂SO₄, and concentrated. The resulting residue was purified by column chromatography (PE/EA=7:1˜3:1) and prep-TLC (PE/EA=2:1) to give the title product (136 mg, 19% for 2 steps) as a colorless solid. MS: M/e 362 (M+1)⁺.

### Step G: methyl 2-(6-amino-4-(2-(furan-2-carbonyl)hydrazinyl)-1H-pyrazolo[3,4-d]pyrimidin-1-yl)-2-(2-methoxyphenyl)propanoate

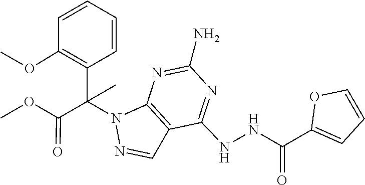 [SMILES: COC(=O)C(C)(c1ccccc1OC)n1ncc2c(NNC(=O)c3ccco3)nc(N)nc21]

A mixture of methyl 2-(6-amino-4-chloro-1H-pyrazolo[3,4-d]pyrimidin-1-yl)-2-(2-methoxyphenyl)propanoate (136 mg, 0.37 mmol), furan-2-carbohydrazide (70 mg, 0.55 mmol) and DIEA (300 mg, 2.32 mmol) in DMSO (2 mL) was heated at 110° C. for 16 hrs. The mixture was poured into 20 mL of H₂O. A light yellow solid precipitated and which was filtered, washed with H₂O (10 mL). The filter cake was dried under lamp to give the title product (52 mg, crude) as a light yellow solid. MS: M/e 452 (M+1)⁺.

### Step H: methyl 2-(5-amino-2-(furan-2-yl)-7H-pyrazolo[4,3-e][1,2,4]triazolo[1,5-c]pyrimidin-7-yl)-2-(2-methoxyphenyl)propanoate

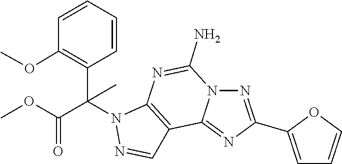 [SMILES: COC(=O)C(C)(c1ccccc1OC)n1ncc2c1nc(N)n1nc(-c3ccco3)nc21]

A mixture of methyl 2-(6-amino-4-(2-(furan-2-carbonyl)hydrazinyl)-1H-pyrazolo[3,4-d]pyrimidin-1-yl)-2-(2-methoxyphenyl)propanoate (52 mg, crude), HMDS (1.5 mL) and BSA (1.5 mL) was heated at 110° C. for 16 hrs. The mixture was concentrated to dryness. The resulting residue was diluted with 10 mL of EA, washed with brine (5 mL×3), dried over Na₂SO₄, concentrated. The resulted residue was purified by prep-TLC (PE/EA=2:1) to give the title product (22 mg, crude) as a light yellow oil. M/e 434 (M+1)⁺.

### Step I: 2-(5-amino-2-(furan-2-yl)-7H-pyrazolo[4,3-e][1,2,4]triazolo[1,5-c]pyrimidin-7-yl)-2-(2-methoxyphenyl)propanoic Acid

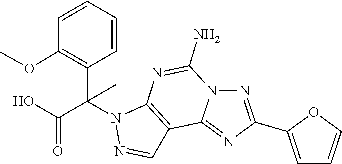 [SMILES: COc1ccccc1C(C)(C(=O)O)n1ncc2c1nc(N)n1nc(-c3ccco3)nc21]

To a solution of methyl 2-(5-amino-2-(furan-2-yl)-7H-pyrazolo[4,3-e][1,2,4]triazolo[1,5-c]pyrimidin-7-yl)-2-(2-methoxyphenyl)propanoate (22 mg, crude) in MeOH was added aqueous solution of NaOH (4 M, 2 mL) at rt and the resulting mixture was stirred at rt for 16 hrs. The mixture was concentrated after having been acidified by HCl (2 M) to pH˜5. The solid was treated with CH₂Cl₂/MeOH (3:1, 20 mL). The suspension was filtered and the filtrate was concentrated to dryness to give the title product (20 mg, crude) as a light yellow solid. M/e 420 (M+1)⁺.

### Step J: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(2-methoxyphenyl)propan-1-one

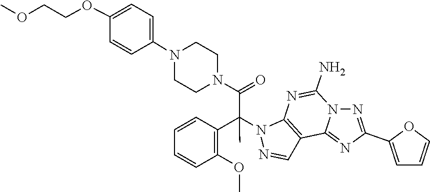 [SMILES: COCCOc1ccc(N2CCN(C(=O)C(C)(c3ccccc3OC)n3ncc4c3nc(N)n3nc(-c5ccco5)nc43)CC2)cc1]

To a mixture of 2-(5-amino-2-(furan-2-yl)-7H-pyrazolo[4,3-e][1,2,4]triazolo[1,5-c]pyrimidin-7-yl)-2-(2-methoxyphenyl)propanoic acid (20 mg, crude), 1-(4-(2-methoxyethoxy)phenyl)piperazine (20 mg, 0.085 mmol), DIEA (50 mg, 0.38 mmol) in DMF (1 mL) was added HATU (27 mg, 0.071 mmol) at rt and the mixture was stirred at rt for 3 hrs. The mixture was diluted with 10 mL of EA, washed with NaHCO₃ (5 mL×2), brine (5 mL×2), dried over Na₂SO₄, concentrated and the resulting oil was purified by prep-TLC (EA: 100%) to give the title product (19.0 mg, yield: 8% for 4 steps) after having been lyophilized. ¹H NMR (400 MHz, DMSO-d6) δ 8.25 (s, 1H), 8.04 (s, 2H), 7.96 (s, 1H), 7.31 (t, J=7.6 Hz, 1H), 7.26 (d, J=3.2 Hz, 1H), 7.09 (d, J=8.0 Hz, 1H), 6.87 (t, J=7.6 Hz, 1H), 6.81-6.64 (m, 6H), 4.00-3.93 (m, 2H), 3.94-3.80 (m, 1H), 3.77 (s, 3H), 3.62-3.57 (m, 2H), 3.51-3.35 (m, 1H), 3.28 (s, 3H), 3.24-2.90 (m, 3H), 2.78-2.61 (m, 2H), 2.39 (s, 3H), 2.04-1.93 (m, 1H). MS: M/e 638 (M+1)⁺.

### Compound A94: 2-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)-N-cyclohexylbenzamide

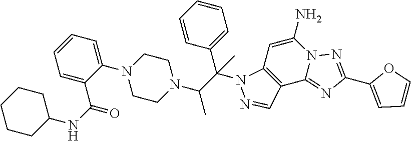 [SMILES: CC(N1CCN(c2ccccc2C(=O)NC2CCCCC2)CC1)C(C)(c1ccccc1)n1ncc2c1cc(N)n1nc(-c3ccco3)nc21]

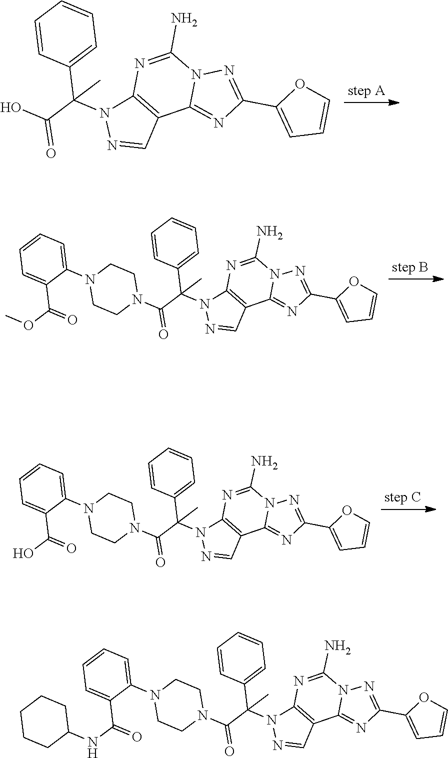 [SMILES: CC(C(=O)O)(c1ccccc1)n1ncc2c1nc(N)n1nc(-c3ccco3)nc21 ; COC(=O)c1ccccc1N1CCN(C(=O)C(C)(c2ccccc2)n2ncc3c2nc(N)n2nc(-c4ccco4)nc32)CC1 ; CC(C(=O)N1CCN(c2ccccc2C(=O)O)CC1)(c1ccccc1)n1ncc2c1nc(N)n1nc(-c3ccco3)nc21 ; CC(C(=O)N1CCN(c2ccccc2C(=O)NC2CCCCC2)CC1)(c1ccccc1)n1ncc2c1nc(N)n1nc(-c3ccco3)nc21]

### Step A: methyl 2-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)benzoate

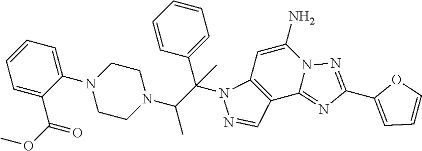 [SMILES: COC(=O)c1ccccc1N1CCN(C(C)C(C)(c2ccccc2)n2ncc3c2cc(N)n2nc(-c4ccco4)nc32)CC1]

A mixture of 2-(5-amino-2-(furan-2-yl)-7H-pyrazolo[4,3-e][1,2,4]triazolo[1,5-c]pyrimidin-7-yl)-2-(2-fluorophenyl)propanoic acid (500 mg, 1.285 mmol), methyl 2-(piperazin-1-yl)benzoate (424 mg, 1.928 mmol), HATU (977 mg, 2.571 mmol), DIPEA (497 mg, 3.856 mmol) in DMF (5 mL) was stirred at rt overnight. The reaction mixture was poured into H₂O (5 mL) and extracted with EtOAc (5 mL×3). The combined organic layers were washed with brine, dried over Na₂SO₄, concentrated and purified by column chromatography (DCM/MeOH=40:1˜10:1) to give the product (603 mg, 79.2%) as white solid. MS: M/e 592 (M+1)⁺.

### Step B: 2-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)benzoic Acid

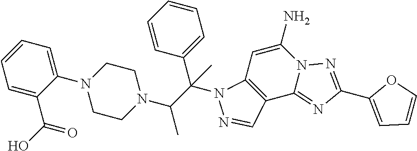 [SMILES: CC(N1CCN(c2ccccc2C(=O)O)CC1)C(C)(c1ccccc1)n1ncc2c1cc(N)n1nc(-c3ccco3)nc21]

A mixture of methyl 2-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)benzoate (603 mg, 1.019 mmol), lithium hydroxide (204 mg, 5.093 mmol) in MeOH (5 mL) and water (2 mL) was stirred at 50° C. overnight. The reaction mixture was acidified with hydrochloric acid, extracted with EtOAc (5 mL×3). The combined organic layers were washed with brine, dried over Na₂SO₄, concentrated and purified by column chromatography (DCM/MeOH=40:1˜10:1) to give the product (490 mg, 83.4%) as white solid. MS: M/e 578 (M+1)⁺.

### Step C: 2-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)-N-cyclohexylbenzamide

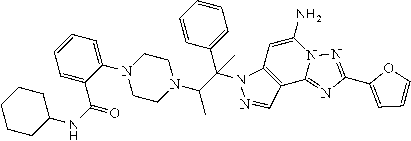 [SMILES: CC(N1CCN(c2ccccc2C(=O)NC2CCCCC2)CC1)C(C)(c1ccccc1)n1ncc2c1cc(N)n1nc(-c3ccco3)nc21]

A mixture of 2-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)benzoic acid (100 mg, 0.1733 mmol), cyclohexanamine (21 mg, 0.2080 mmol), HATU (79 mg, 0.2080 mmol), DIPEA (67 mg, 0.5199 mmol) in DMF (3 mL) was stirred at rt for 3 hours. The reaction mixture was poured into H₂O (5 mL) and extracted with DCM (5 mL×3). The combined organic layers were washed with brine, dried over Na₂SO₄, concentrated and purified by column chromatography (DCM/MeOH=40:1˜10:1) to give the product (85 mg, 74.54%) as white solid. ¹H NMR (400 MHz, DMSO-d6) δ 8.82 (d, J=7.8 Hz, 1H), 8.32 (s, 1H), 8.14 (s, 2H), 7.96 (s, 1H), 7.59 (d, J=7.6 Hz, 1H), 7.37-7.27 (m, 4H), 7.27-7.17 (m, 3H), 7.09 (t, J=7.4 Hz, 1H), 7.01 (d, J=8.0 Hz, 1H), 6.74 (dd, J=3.0, 1.6 Hz, 1H), 3.72 (s, 2H), 3.62 (dd, J=11.1, 7.2 Hz, 2H), 2.92 (s, 5H), 2.33 (s, 3H), 1.73 (d, J=22.9 Hz, 2H), 1.58 (t, J=13.1 Hz, 2H), 1.45 (d, J=12.6 Hz, 1H), 1.25-1.11 (m, J=14.6, 10.4 Hz, 2H), 1.09-0.90 (m, 3H). MS: M/e 659 (M+1)⁺.

### Compound A95: 2-(5-amino-2-(3-methylpyrazin-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-phenylpropan-1-one

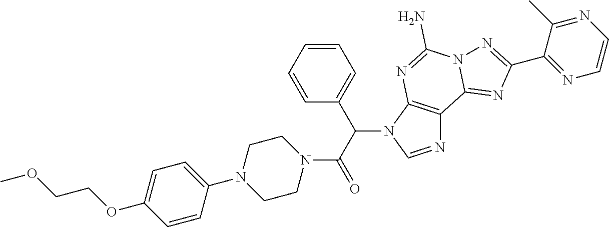 [SMILES: COCCOc1ccc(N2CCN(C(=O)C(c3ccccc3)n3cnc4c3nc(N)n3nc(-c5nccnc5C)nc43)CC2)cc1]

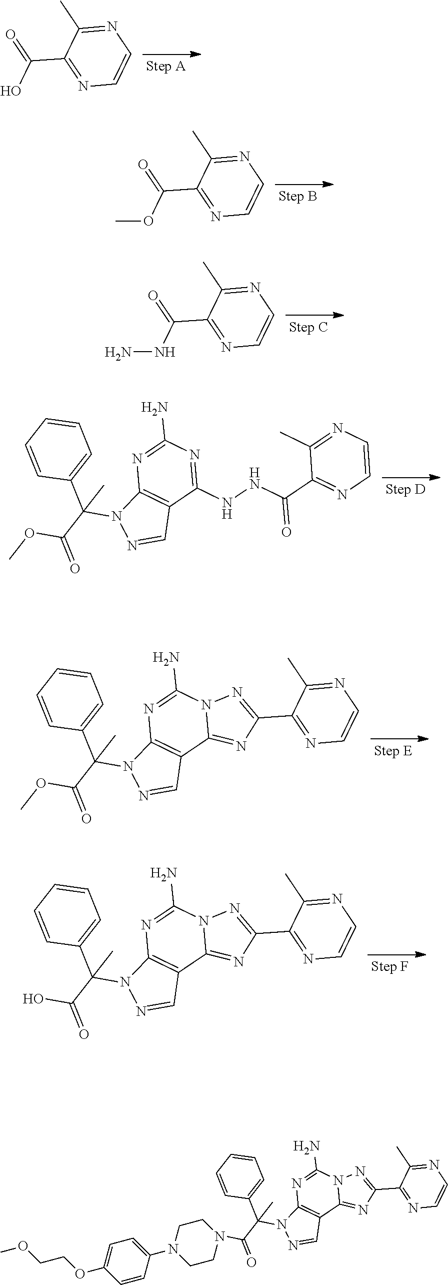

### Step A: methyl 3-methylpyrazine-2-carboxylate

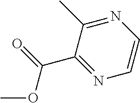

To a stirred solution of MeOH (50 mL) was added SOCl₂ (8.6 g, 72.4 mmol) dropwise at 0° C. Then 3-methylpyrazine-2-carboxylic acid (5 g, 36.2 mmol) was added to the reaction. The mixture was stirred at 60° C. overnight. The mixture was concentrated under reduced pressure and the residue was dissolved into EA (50 mL). The organic phase was washed with saturated aq.NaHCO3 (50 mL). The organic phase was washed with brine, dried over Na₂SO₄ and concentrated under reduced pressure. The residue was purified by silica gel chromatography (eluted with: EA/PE=1/1) to afford the title compound as yellow solid (3.9 g, yield: 70.9%). MS: M/e 153 (M+1)⁺.

### Step B: 3-methylpyrazine-2-carbohydrazide

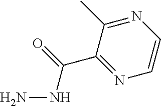

To a stirred solution of the product of Step A (0.9 g, 5.9 mmol) in MeOH (20 mL) was added hydrazine hydrate (1.5 g, 23.7 mmol) at RT. The mixture was stirred at 60° C. overnight. The mixture was concentrated and the residue was dissolved into EA (30 mL). The organic phase was washed with brine, dried over Na₂SO₄ and concentrated under reduced pressure. The residue (460 mg, yield: 51.1%) was used into next step directly. MS: M/e 153 (M+1)⁺.

### Step C: methyl 2-(6-amino-4-(2-(3-methylpyrazine-2-carbonyl)hydrazinyl)-1H-pyrazolo[3,4-d]pyrimidin-1-yl)-2-phenylpropanoate

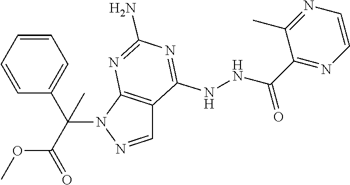 [SMILES: COC(=O)C(C)(c1ccccc1)n1ncc2c(NNC(=O)c3nccnc3C)nc(N)nc21]

A mixture of methyl 2-(6-amino-4-chloro-1H-pyrazolo[3,4-d]pyrimidin-1-yl)-2-phenylpropanoate (1 g, 3 mmol), the product of Step B (460 mg, 3 mmol) and Et₃N (610 mg, 6 mmol) in DMSO (10 mL) was stirred at 100° C. overnight. The reaction was cooled to RT. The mixture was poured into water (30 mL). The precipitate was formed from the system. After stirring at RT for 30 mins, the mixture was filtered. The solid was collected and dried in air. The yellow solid (1.6 g, crude) was used into next step directly. MS: M/e 448 (M+1)⁺.

### Step D: methyl 2-(5-amino-2-(3-methylpyrazin-2-yl)-7H-pyrazolo[4,3-e][1,2,4]triazolo[1,5-c]pyrimidin-7-yl)-2-phenylpropanoate

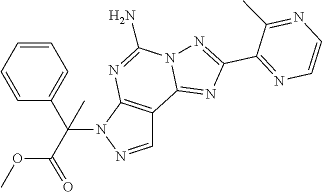 [SMILES: COC(=O)C(C)(c1ccccc1)n1ncc2c1nc(N)n1nc(-c3nccnc3C)nc21]

A mixture of the product of Step C (1 g, 2.2 mmol) in BSA (5 mL) and HMDS (5 mL) was stirred at 100° C. overnight. The reaction was cooled to RT and concentrated under reduced pressure. The residue was dissolved into H₂O (10 mL) and MeOH (10 mL). The mixture was stirred at 80° C. for 2 hours. MeOH was removed and the solid was precipitated from the system. The solid was filtered and dried in air. The brown solid (300 mg, yield for two steps: 37.5%) was used into next step directly. MS: M/e 430 (M+1)⁺.

### Step E: 2-(5-amino-2-(3-methylpyrazin-2-yl)-7H-pyrazolo[4,3-e][1,2,4]triazolo[1,5-c]pyrimidin-7-yl)-2-phenylpropanoic Acid

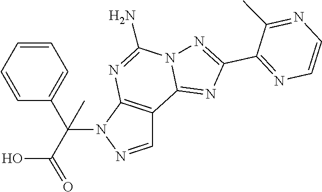 [SMILES: Cc1nccnc1-c1nc2c3cnn(C(C)(C(=O)O)c4ccccc4)c3nc(N)n2n1]

To a stirred solution of the product of step D in MeOH (6 mL) was added aq.NaOH (2 mL) at RT. The mixture was stirred at RT overnight. The solvents were removed and the residue was dissolved into water (20 mL). The mixture was acidified to pH=3˜4 with aq. HCl (2M). The solid was precipitated from the system. The mixture was filtered and the solid was collected. The white solid was dried in air and used into next step directly. MS: M/e 416 (M+1)⁺.

### Step F: 2-(5-amino-2-(3-methylpyrazin-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-phenylpropan-1-one

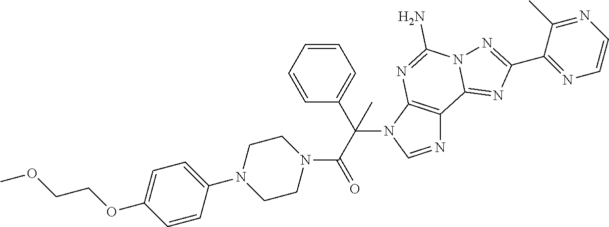 [SMILES: COCCOc1ccc(N2CCN(C(=O)C(C)(c3ccccc3)n3cnc4c3nc(N)n3nc(-c5nccnc5C)nc43)CC2)cc1]

A mixture of 1-(4-(2-methoxyethoxy)phenyl)piperazine (102 mg, 0.43 mmol), the product of step E (180 mg, 0.43 mmol), HATU (179 mg, 0.47 mmol) and Et₃N (0.1 mL, excess) in DMF (2 mL) was stirred at 0° C. for 2 hours. The reaction was then warmed to RT and stirred overnight. The reaction mixture was poured into water (10 mL) and extracted with EA (15 mL×3). The combined organic layers were washed with brine, dried over Na₂SO₄ and concentrated under reduced pressure. The residue was purified by prep-TLC (EA: 100%) to afford the title compound as white solid (15 mg, yield: 5.2%). ¹H NMR (400 MHz, DMSO-d6) δ 8.70 (d, J=4 Hz, 2H), 8.40 (s, 1H), 8.26 (br.s, 2H), 7.53-7.40 (m, 2H), 7.38-7.26 (m, 3H), 7.17 (d, J=8 Hz, 2H), 7.00 (d, J=8.0 Hz, 2H), 4.11-4.02 (m, 2H), 3.69-3.60 (m, 2H), 3.54-3.36 (m, 2H), 3.28 (s, 3H), 3.23-2.97 (m, 2H), 2.89 (s, 3H), 2.56-2.50 (m, 4H), 2.41 (s, 3H) ppm. MS: M/e 634 (M+1)⁺.

### Compound A96: 2-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)-N,N-dimethylbenzamide

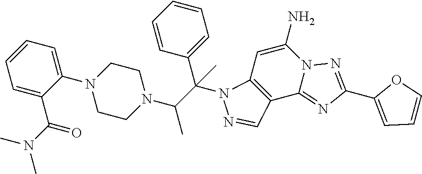 [SMILES: CC(N1CCN(c2ccccc2C(=O)N(C)C)CC1)C(C)(c1ccccc1)n1ncc2c1cc(N)n1nc(-c3ccco3)nc21]

A mixture of 2-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)benzoic acid (100 mg, 0.1733 mmol), dimethylamine hydrochloride (17 mg, 0.2080 mmol), HATU (79 mg, 0.2080 mmol), DIPEA (67 mg, 0.5199 mmol) in DMF (3 mL) was stirred at rt for 3 hours. The reaction mixture was poured into H₂O (5 mL) and extracted with DCM (5 mL×3). The combined organic layers were washed with brine, dried over Na₂SO₄, concentrated and purified by column chromatography (DCM/MeOH=40:1˜10:1) to give the product (73 mg, 69.74%). ¹H NMR (400 MHz, DMSO-d6) δ 8.32 (d, J=6.6 Hz, 1H), 8.14 (s, 2H), 7.96 (s, 1H), 7.37-7.20 (m, 7.2 Hz, 6H), 7.18 (d, J=7.1 Hz, 1H), 7.06-6.95 (m, 2H), 6.86 (t, J=8.9 Hz, 1H), 6.74 (s, 1H), 3.62-3.37 (m, 4H), 3.13-2.69 (m, 8H), 2.32 (s, 3H), 1.47-1.26 (m, 6H). MS: M/e 605 (M+1)⁺.

### Compound A97: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-methyl-3-phenylpropan-1-one

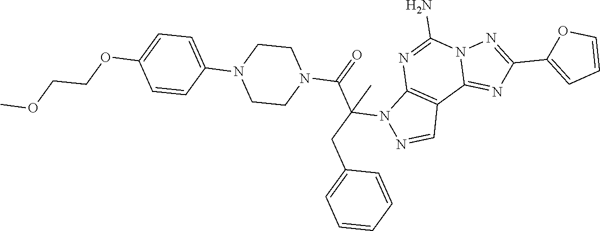 [SMILES: COCCOc1ccc(N2CCN(C(=O)C(C)(Cc3ccccc3)n3ncc4c3nc(N)n3nc(-c5ccco5)nc43)CC2)cc1]

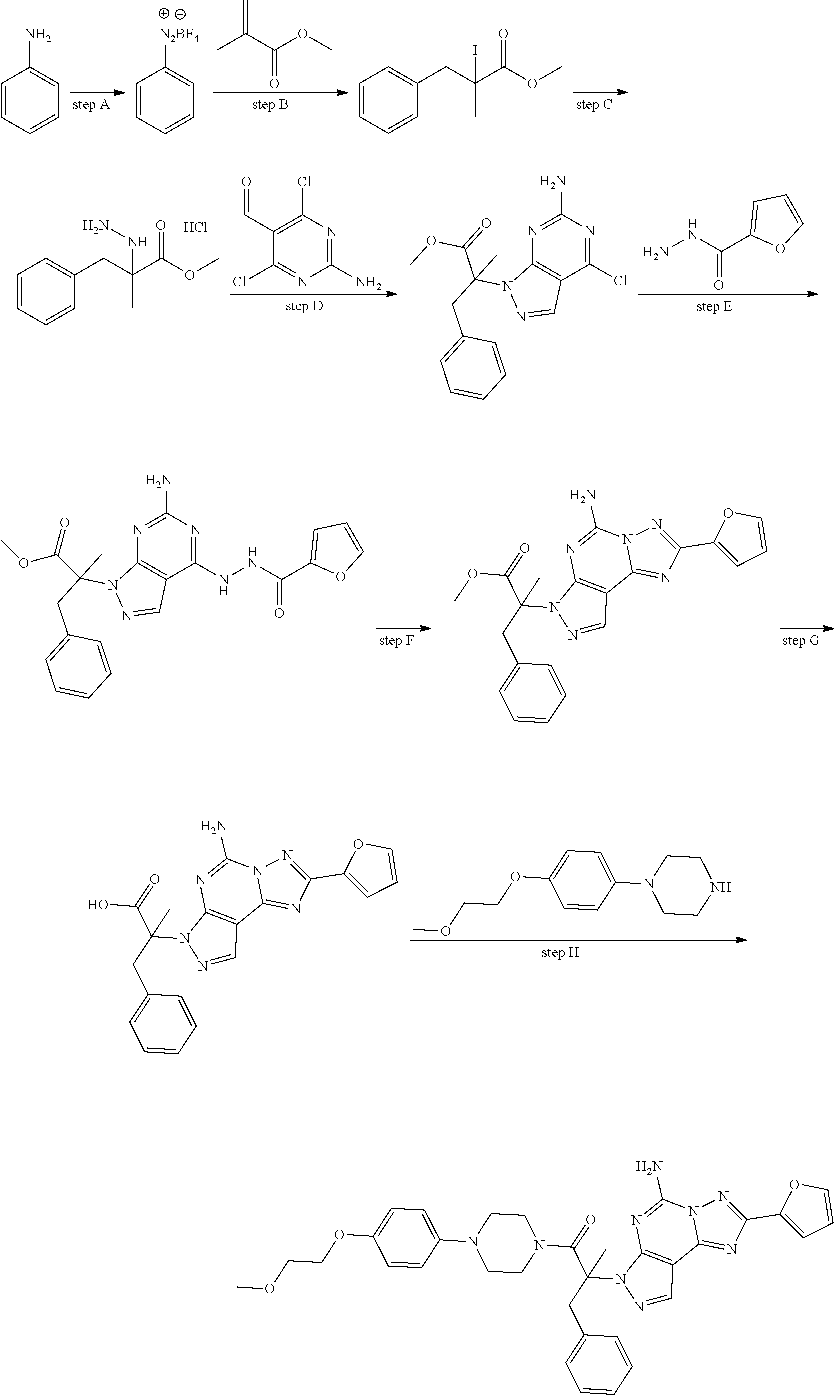

### Step A: Benzenediazonium Tetrafluoroborate

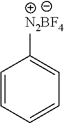

To a stirred solution of aniline (5 g, 0.054 mol) in HBF4 (aq., 30%, 20 ml) and H₂O (20 ml) was added a solution of NaNO₂ (7.4 g, 0.11 mol) in H₂O (20 ml) at 0° C. The reaction mixture was stirred at 0° C. for 1 h. After completion, the precipitate was filtered. The filtration cake was recrystallized with acetone and MTBE to afford the product (7.6 g, 74%) as a white solid.

### Step B: methyl 2-iodo-2-methyl-3-phenylpropanoate

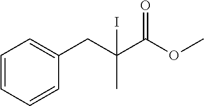

To a stirred solution of benzenediazonium tetrafluoroborate (7 g, 36.5 mmol) and methyl methacrylate (4.0 g, 40.0 mmol) in DMF (30 ml) was added a solution of KI (6.7 g, 40.4 mmol) in H₂O (15 ml) at 0˜10° C. The reaction mixture was stirred at 0˜20° C. for 3 h. After completion, the reaction mixture was poured into water (30 ml) and then extracted with EA (30 ml×3). The combined organic layer was washed with water (20 ml), dried over Na₂SO₄ and concentrated under reduced pressure to afford a residue. The residue was purified by column chromatography with PE:EA (20:1) to afford the product (4.8 g, 43%) as a light-yellow liquid. ¹H NMR (400 MHz, CDCl₃) δ 7.32-7.26 (m, 3H), 7.23-7.16 (m, 2H), 3.79 (s, 3H), 3.75 (d, J=8 Hz, 1H), 3.45 (d, J=8 Hz, 1H), 2.00 (s, 3H) ppm

### Step C: methyl 2-hydrazinyl-2-methyl-3-phenylpropanoate Hydrochloride

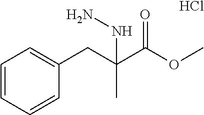

A solution of methyl 2-iodo-2-methyl-3-phenylpropanoate (4.2 g, 13.8 mmol) and hydrazine hydrate (3.45 g, 55.2 mmol) in MeCN (35 ml) was stirred at 25° C. for 24 h. After completion, the reaction mixture was concentrated under reduced pressure. The residue was diluted with EA (35 ml) and then washed with H₂O (20 ml×2) to remove hydrazine hydrate. The organic layer was concentrated to afford a residue. The residue was diluted with HCl (aq., 1M, 30 ml) and then washed with EA (20 ml×2). The aqueous layer was lyophilized to afford the product (1.2 g, 36%) as a white solid. MS: M/e 209 (M+1)⁺.

### Step D: methyl 2-(6-amino-4-chloro-1H-pyrazolo[3,4-d]pyrimidin-1-yl)-2-methyl-3-phenylpropanoate

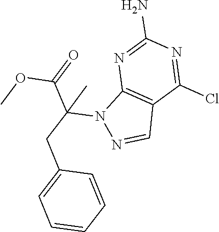 [SMILES: COC(=O)C(C)(Cc1ccccc1)n1ncc2c(Cl)nc(N)nc21]

A solution of methyl 2-hydrazinyl-2-methyl-3-phenylpropanoate hydrochloride (1.1 g, 5.29 mmol) and 2-amino-4,6-dichloropyrimidine-5-carbaldehyde (1.0 g, 5.29 mmol) in MeCN (25 ml) was stirred at rt overnight and then 70° C. for 3 h. After completion, the reaction mixture was filtered. The filtrate was concentrated under reduced pressure to afford a residue. The residue was purified by column chromatography with PE:EA (10:1) to afford the product (0.57 g, 31%) as a yellow solid. MS: M/e 346 (M+1)⁺.

### Step E: methyl 2-(6-amino-4-(2-(furan-2-carbonyl)hydrazinyl)-1H-pyrazolo[3,4-d]pyrimidin-1-yl)-2-methyl-3-phenylpropanoate

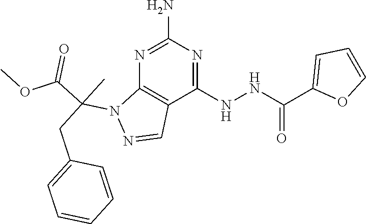 [SMILES: COC(=O)C(C)(Cc1ccccc1)n1ncc2c(NNC(=O)c3ccco3)nc(N)nc21]

A solution of methyl 2-(6-amino-4-chloro-1H-pyrazolo[3,4-d]pyrimidin-1-yl)-2-methyl-3-phenylpropanoate (0.55 g, 1.59 mmol), furan-2-carbohydrazide (0.22 g, 1.75 mmol) and DIEA (0.41 g, 3.18 mmol) in DMSO (10 ml) was stirred at 110° C. overnight. After completion, the reaction mixture was concentrated under reduced pressure to afford a residue. The residue was purified by column chromatography with PE:EA (1:10) to afford the product (0.30 g, 43%) as a yellow solid. MS: M/e 436 (M+1)⁺.

### Step F: methyl 2-(5-amino-2-(furan-2-yl)-7H-pyrazolo[4,3-e][1,2,4]triazolo[1,5-c]pyrimidin-7-yl)-2-methyl-3-phenylpropanoate

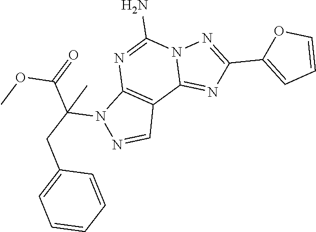 [SMILES: COC(=O)C(C)(Cc1ccccc1)n1ncc2c1nc(N)n1nc(-c3ccco3)nc21]

A solution of methyl 2-(6-amino-4-(2-(furan-2-carbonyl)hydrazinyl)-1H-pyrazolo[3,4-d]pyrimidin-1-yl)-2-methyl-3-phenylpropanoate (0.30 g, 0.69 mmol) in BSA (5 ml) and HMDS (5 ml) was stirred at 110° C. overnight. After completion, the reaction mixture was concentrated under reduced pressure to afford a residue. The residue was diluted with NaHCO₃ (aq., 20 ml) and then extracted with EA (20 ml×2). The combined organic layer was dried over Na₂SO₄, filtered, concentrated and then purified by prep-TLC with PE:EA (1:1) to afford the product (0.15 g, 51%) as a yellow solid. MS: M/e 418 (M+1)⁺.

### Step G: 2-(5-amino-2-(furan-2-yl)-7H-pyrazolo[4,3-e][1,2,4]triazolo[1,5-c]pyrimidin-7-yl)-2-methyl-3-phenylpropanoic Acid

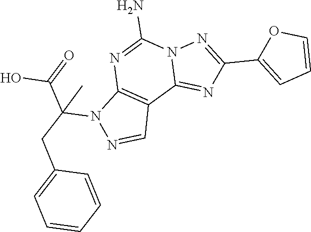 [SMILES: CC(Cc1ccccc1)(C(=O)O)n1ncc2c1nc(N)n1nc(-c3ccco3)nc21]

A solution of methyl 2-(5-amino-2-(furan-2-yl)-7H-pyrazolo[4,3-e][1,2,4]triazolo[1,5-c]pyrimidin-7-yl)-2-methyl-3-phenylpropanoate (0.15 g, 0.36 mmol) and NaOH (0.14 g, 3.60 mmol) in EtOH (10 ml) and H₂O (2 ml) was stirred at 70° C. for 2 h. After completion, the reaction mixture was concentrated under reduced pressure to remove EtOH. The residue was diluted with H₂O (10 ml) and then acidified with HCl (aq., 4M) to pH=3˜4. The precipitate was filtered, washed with water and then dried to afford the product (0.10 g, 69%) as a yellow solid. MS: M/e 404 (M+1)⁺.

### Step H: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-methyl-3-phenylpropan-1-one

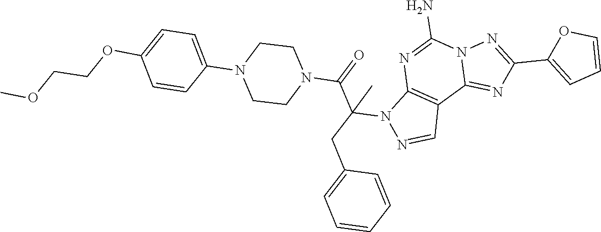 [SMILES: COCCOc1ccc(N2CCN(C(=O)C(C)(Cc3ccccc3)n3ncc4c3nc(N)n3nc(-c5ccco5)nc43)CC2)cc1]

To a stirred solution of 2-(5-amino-2-(furan-2-yl)-7H-pyrazolo[4,3-e][1,2,4]triazolo[1,5-c]pyrimidin-7-yl)-2-methyl-3-phenylpropanoic acid (0.10 g, 0.25 mmol), HATU (0.10 g, 0.26 mmol) and DIEA (0.10 g, 0.78 mmol) in THF (15 ml) was added 1-(4-(2-methoxyethoxy)phenyl)piperazine (0.059 g, 0.25 mmol). The reaction mixture was stirred at rt for 15 h. After completion, the reaction mixture was diluted with EA (30 ml) and then washed with H₂O (15 ml×2). The organic layer was dried over Na₂SO₄, filtered and then concentrated under reduced pressure to afford a residue. The residue was purified by prep-TLC with PE:EA (1:3) to afford the product (13 mg). ¹H NMR (400 MHz, DMSO-d6) δ 8.14 (s, 3H), 7.95 (s, 1H), 7.22 (d, J=4 Hz, 1H), 7.15-7.05 (m, 3H), 6.78-6.63 (m, 7H), 3.96-3.89 (m, 3H), 3.81-3.58 (m, 2H), 3.56 (t, J=4 Hz, 2H), 3.54-3.42 (m, 5H), 3.25 (s, 3H), 3.09-2.90 (m, 2H), 1.79 (s, 3H) ppm. MS: M/e 622 (M+1)⁺.

### Compound A98: 4-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)-N-cyclohexylbenzamide

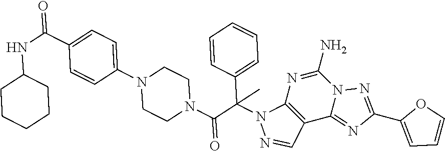 [SMILES: CC(C(=O)N1CCN(c2ccc(C(=O)NC3CCCCC3)cc2)CC1)(c1ccccc1)n1ncc2c1nc(N)n1nc(-c3ccco3)nc21]

To a stirred solution of 4-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)benzoic acid (80 mg, 0.14 mmol), HATU (79 mg, 0.21 mmol) and DIPEA (54 mg, 0.42 mmol) in THF (15 ml) was added cyclohexanamine (21 mg, 0.21 mmol). The reaction mixture was stirred at rt for 15 h. After completion, the reaction mixture was diluted with EA (30 ml) and then washed with H₂O (15 ml×2). The organic layer was dried over Na₂SO₄, filtered and then concentrated under reduced pressure to afford a residue. The residue was purified by prep-TLC with DCM:MeOH (10:1) to afford the product (15.3 mg, 17%). ¹H NMR (400 MHz, DMSO-d6) δ 8.32 (s, 1H), 8.08 (s, 2H), 7.95 (s, 1H), 7.84 (d, J=8 Hz, 1H), 7.65 (d, J=8 Hz, 2H), 7.38-7.16 (m, 6H), 6.79 (d, J=8 Hz, 2H), 6.74 (s, 1H), 3.90-3.55 (m, 3H), 3.25-3.11 (m, 2H), 3.10-2.72 (m, 4H), 2.34 (s, 3H), 1.79-1.65 (m, 4H), 1.60-1.54 (m, 1H), 1.31-1.22 (m, 5H) ppm. MS: M/e 659 (M+1)⁺.

### Compound A99: 2-(5-amino-2-(furan-2-yl)-7H-pyrazolo[4,3-e][1,2,4]triazolo[1,5-c]pyrimidin-7-yl)-2-phenyl-1-(4-(2-(piperidine-1-carbonyl)phenyl)piperazin-1-yl)propan-1-one

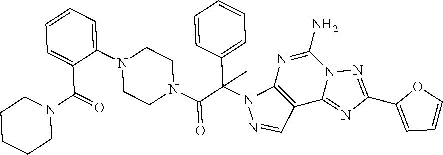 [SMILES: CC(C(=O)N1CCN(c2ccccc2C(=O)N2CCCCC2)CC1)(c1ccccc1)n1ncc2c1nc(N)n1nc(-c3ccco3)nc21]

A mixture of 2-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)benzoic acid (100 mg, 0.1733 mmol), piperidine (18 mg, 0.2080 mmol), HATU (79 mg, 0.2080 mmol), DIPEA (67 mg, 0.5199 mmol) in DMF (3 mL) was stirred at rt for 3 hours. The reaction mixture was poured into H₂O (5 mL) and extracted with DCM (5 mL×3). The combined organic layers were washed with brine, dried over Na₂SO₄, concentrated and purified by column chromatography (DCM/MeOH=40:1˜10:1) to give the product (82 mg, 73.47%) as white solid. ¹H NMR (400 MHz, DMSO-d6) δ 8.32 (d, J=6.6 Hz, 1H), 8.14 (s, 2H), 7.96 (s, 1H), 7.37-7.20 (m, 6H), 7.18 (d, J=7.1 Hz, 1H), 7.06-6.95 (m, 2H), 6.86 (t, J=8.9 Hz, 1H), 6.74 (s, 1H), 3.82 (s, 1H), 3.62-3.37 (m, 4H), 3.19-2.56 (m, 65.0 Hz, 10H), 2.32 (s, 3H). MS: M/e 645 (M+1)⁺.

### Compound A100: 2-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)-N-((3-hydroxyoxetan-3-yl)methyl)benzamide

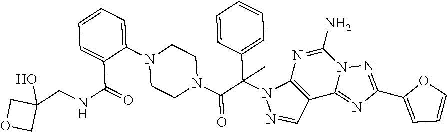 [SMILES: CC(C(=O)N1CCN(c2ccccc2C(=O)NCC2(O)COC2)CC1)(c1ccccc1)n1ncc2c1nc(N)n1nc(-c3ccco3)nc21]

A mixture of 2-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)benzoic acid (100 mg, 0.1733 mmol), 3-(aminomethyl)oxetan-3-ol (21 mg, 0.2080 mmol), HATU (79 mg, 0.2080 mmol), DIPEA (67 mg, 0.5199 mmol) in DMF (3 mL) was stirred at rt for 3 hours. The reaction mixture was poured into H₂O (5 mL) and extracted with DCM (5 mL×3). The combined organic layers were washed with brine, dried over Na₂SO₄, concentrated and purified by column chromatography (DCM/MeOH=40:1˜10:1) to give the product (82 mg, 73.47%) as white solid. ¹H NMR (400 MHz, DMSO-d6) δ 9.49 (s, 1H), 8.30 (s, 1H), 8.10 (s, 2H), 7.96 (s, 1H), 7.77 (d, J=7.5 Hz, 1H), 7.39-7.26 (m, 7.1 Hz, 4H), 7.26-7.17 (m, 3H), 7.14 (t, J=7.4 Hz, 1H), 6.97 (d, J=8.0 Hz, 1H), 6.74 (s, 1H), 5.97 (s, 1H), 4.35-4.24 (m, 4H), 3.76 (s, 2H), 3.57 (s, 2H), 3.05 (s, 2H), 2.89 (s, 4H), 2.35 (s, 3H). MS: M/e 645 (M+1)⁺.

### Compound A101: 2-(5-amino-2-(furan-2-yl)-7H-pyrazolo[4,3-e][1,2,4]triazolo[1,5-c]pyrimidin-7-yl)-2-phenyl-1-(4-(4-(2-(pyrrolidin-1-yl)ethoxy)phenyl)piperazin-1-yl)propan-1-one

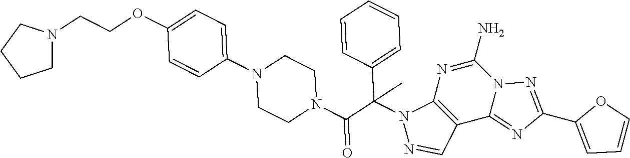 [SMILES: CC(C(=O)N1CCN(c2ccc(OCCN3CCCC3)cc2)CC1)(c1ccccc1)n1ncc2c1nc(N)n1nc(-c3ccco3)nc21]

To a stirred solution of 2-(5-amino-2-(furan-2-yl)-7H-pyrazolo[4,3-e][1,2,4]triazolo[1,5-c]pyrimidin-7-yl)-2-phenylpropanoic acid (70 mg, 0.18 mmol), HATU (75 mg, 0.20 mmol) and DIEA (70 mg, 0.54 mmol) in THF (15 ml) was added 1-(4-(2-(pyrrolidin-1-yl)ethoxy)phenyl)piperazine (62 mg, 0.20 mmol). The reaction mixture was stirred at rt for 15 h. After completion, the reaction mixture was diluted with EA (30 ml) and then washed with H₂O (15 ml×2). The organic layer was dried over Na₂SO₄, filtered and then concentrated under reduced pressure to afford a residue. The residue was purified by prep-TLC with DCM:MeOH (5:1) to afford the product (35.9 mg, 31%). ¹H NMR (400 MHz, DMSO-d6) δ 8.32 (s, 1H), 8.09 (s, 2H), 7.96 (s, 1H), 7.37-7.28 (m, 3H), 7.27-7.15 (m, 3H), 6.82 (d, J=8 Hz, 2H), 6.76 (d, J=8 Hz, 3H), 4.17 (t, J=4 Hz, 2H), 3.91-3.60 (m, 2H), 3.59-3.39 (m, 4H), 3.31-3.29 (m, 1H), 3.23-2.70 (m, 7H), 2.33 (s, 3H), 2.00-1.80 (m, 4H) ppm. MS: M/e 647 (M+1)⁺.

### Compound A102: 2-(5-amino-2-(furan-2-yl)-7H-pyrazolo[4,3-e][1,2,4]triazolo[1,5-c]pyrimidin-7-yl)-2-phenyl-1-(4-(4-(piperidine-1-carbonyl)phenyl)piperazin-1-yl)propan-1-one

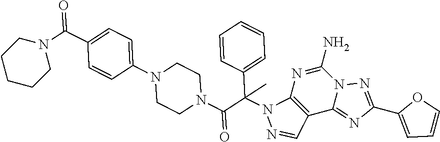 [SMILES: CC(C(=O)N1CCN(c2ccc(C(=O)N3CCCCC3)cc2)CC1)(c1ccccc1)n1ncc2c1nc(N)n1nc(-c3ccco3)nc21]

To a stirred solution of 4-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)benzoic acid (80 mg, 0.14 mmol), HATU (79 mg, 0.21 mmol) and DIPEA (54 mg, 0.42 mmol) in THF (15 ml) was added piperidine (18 mg, 0.21 mmol). The reaction mixture was stirred at rt for 15 h. After completion, the reaction mixture was diluted with EA (30 ml) and then washed with H₂O (15 ml×2). The organic layer was dried over Na₂SO₄, filtered and then concentrated under reduced pressure to afford a residue. The residue was purified by prep-TLC with DCM:MeOH (10:1) to afford the product (4.5 mg). ¹H NMR (400 MHz, DMSO-d6) δ 8.32 (s, 1H), 8.08 (s, 2H), 7.95 (s, 1H), 7.35-7.29 (m, 3H), 7.23 (d, J=4 Hz, 1H), 7.18 (t, J=8 Hz, 4H), 6.79 (d, J=12 Hz, 2H), 6.74 (dd, J=4 Hz, 2 Hz, 1H), 3.90-3.52 (m, 2H), 3.42-3.37 (m, 3H), 3.31 (s, 2H), 3.27-2.85 (m, 5H), 2.34 (s, 3H), 1.60-1.55 (m, 2H), 1.49-1.41 (m, 4H) ppm. MS: M/e 645 (M+1)⁺.

### Compound A103: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(o-tolyl)propan-1-one

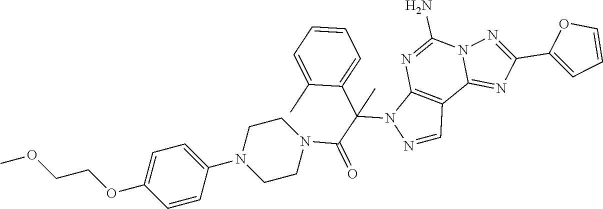 [SMILES: COCCOc1ccc(N2CCN(C(=O)C(C)(c3ccccc3C)n3ncc4c3nc(N)n3nc(-c5ccco5)nc43)CC2)cc1]

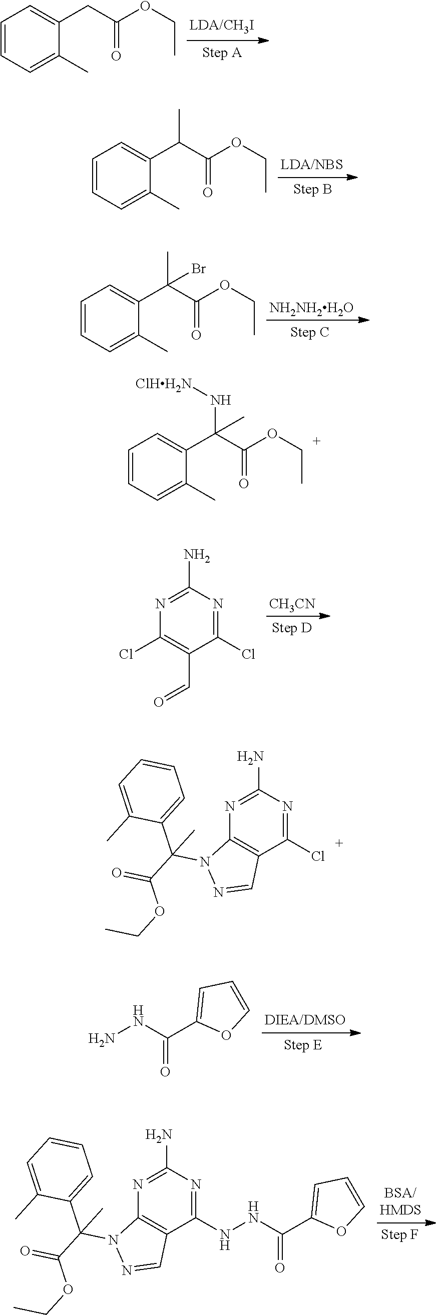

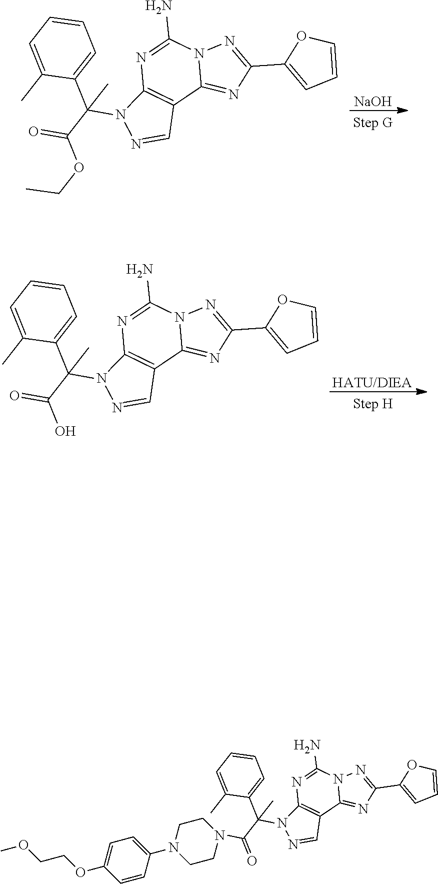

### Step A: ethyl 2-(o-tolyl)propanoate

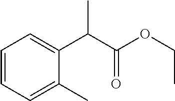

To a cooled solution of LDA (2M in THF, 16.7 mL, 33.4 mmol) in THF (80 mL) at −78° C. under N₂ atmosphere was added with a solution of ethyl 2-(o-tolyl)acetate (5.4 g, 30.3 mmol) in THF (10 mL) dropwise. After addition, the solution was stirred for further 20 mins before CH₃I (12.9 g, 90.9 mmol) was added dropwise. The mixture was allowed to warm to rt overnight and then quenched with water (30 mL). The aqueous layer was extracted with ethyl acetate (100 mL). The organic layer was washed with brine (80 mL), dried, concentrated and purified by column chromatography (PE:EA=50:1) to get the product as an colorless oil (5.5 g, 95%). ¹H NMR (400 MHz, CDCl₃) δ 7.27-7.13 (m, 4H), 4.16-4.07 (m, 2H), 3.96-3.91 (m, 1H), 2.37 (s, 3H), 1.46 (d, J=8.0 Hz, 3H), 1.19 (t, J=8.0 Hz, 3H) ppm.

### Step B: ethyl 2-bromo-2-(o-tolyl)propanoate

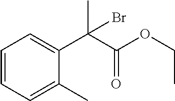

To a cooled solution of LDA (2M in THF, 11.5 mL, 23 mmol) in THF (130 mL) at −78° C. under N₂ atmosphere was added with a solution of ethyl 2-(o-tolyl)propanoate (4 g, 20.8 mmol) in THF (10 mL) dropwise. The solution was stirred for further 30 mins before the dropwise addition of TMSCl (2.5 g, 23 mmol). The mixture was allowed to warm to rt for 2 hrs before being cooled to −78° C. NBS (7.4 g, 41.6 mmol) was added in portions and the solution was stirred at −78° C. for 1 hr. The reaction mixture was allowed to reach to rt over 1 hr and stirred for 2 hrs. After quenched with saturated NaHCO₃ solution, the solution was extracted with ethyl acetate (100 mL). The organic layer was dried, concentrated to get the crude product (5.6 g, crude), which was used in the next step directly.

### Step C: ethyl 2-hydrazinyl-2-(o-tolyl)propanoate Hydrochloride

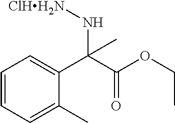

NH₂NH₂.H₂O (10 mL, 0.2 mol) was added to a solution of ethyl 2-bromo-2-(o-tolyl)propanoate (5.6 g, 20.8 mmol) in CH₃CN (50 mL). The reaction mixture was heated at 60° C. overnight. The solution was concentrated, added with water (20 mL) and extracted with ethyl acetate (30 mL×3). The combined organic layers were dried, concentrated, added with HCl/EA (2 M, 5 mL) and evaporated to get the product as HCl salt (3.6 g, 67%). MS: M/e 223 (M+1)⁺

### Step D: ethyl 2-(6-amino-4-chloro-1H-pyrazolo[3,4-d]pyrimidin-1-yl)-2-(o-tolyl)propanoate

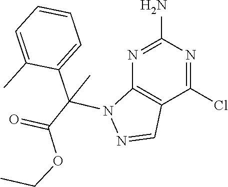 [SMILES: CCOC(=O)C(C)(c1ccccc1C)n1ncc2c(Cl)nc(N)nc21]

A mixture of 2-amino-4,6-dichloropyrimidine-5-carbaldehyde (2.6 g, 13.8 mmol) and ethyl 2-hydrazinyl-2-(o-tolyl)propanoate (3.6 g, 13.8 mmol) in CH₃CN (35 mL) was stirred at rt overnight, and then heated at 50° C. for 3 hrs. The reaction mixture was filtered. The filtrate was evaporated, added with water (20 mL) and extracted with ethyl acetate (20 mL). The organic phase was dried, concentrated and further purified by column chromatography (PE:EA=5:1) to get the desired product as a white solid (1.2 g, 25%). MS: M/e 360 (M+1)⁺

### Step E: ethyl 2-(6-amino-4-(2-(furan-2-carbonyl)hydrazinyl)-1H-pyrazolo[3,4-d]pyrimidin-1-yl)-2-(o-tolyl)propanoate

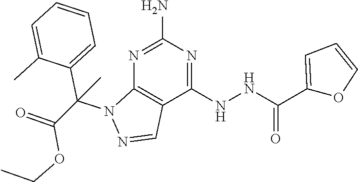 [SMILES: CCOC(=O)C(C)(c1ccccc1C)n1ncc2c(NNC(=O)c3ccco3)nc(N)nc21]

A mixture of ethyl 2-(6-amino-4-chloro-1H-pyrazolo[3,4-d]pyrimidin-1-yl)-2-(o-tolyl)propanoate (1.2 g, 3.3 mmol), furan-2-carbohydrazide (416 mg, 3.3 mmol) and DIEA (852 mg, 6.6 mmol) in DMSO (15 mL) was heated at 115° C. overnight. The solvent was evaporated under oil pump. The residue was added with water (10 mL), slurried and filtered. The cake was washed with water, dried to get the crude product, which was used in the next step directly (1.1 g, 73%). MS: M/e 450 (M+1)⁺

### Step F: ethyl 2-(5-amino-2-(furan-2-yl)-7H-pyrazolo[4,3-e][1,2,4]triazolo[1,5-c]pyrimidin-7-yl)-2-(o-tolyl)propanoate

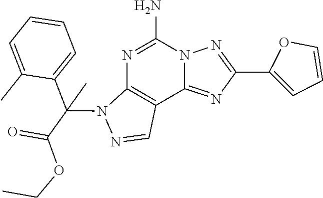 [SMILES: CCOC(=O)C(C)(c1ccccc1C)n1ncc2c1nc(N)n1nc(-c3ccco3)nc21]

A solution of ethyl 2-(6-amino-4-(2-(furan-2-carbonyl)hydrazinyl)-1H-pyrazolo[3,4-d]pyrimidin-1-yl)-2-(o-tolyl)propanoate (400 mg, 1 mmol) in BSA (2 mL) and HMDS (2 mL) was heated at 120° C. for 3 hrs. The solvent was evaporated under oil pump. The residue was added with water (5 mL), extracted with ethyl acetate (15 mL) and washed with brine (10 mL). The organic layer was dried, concentrated and purified by column chromatography (PE:EA=10:1 to 3:1) to get the desired product as a white solid (50 mg, 13%). MS: M/e 432 (M+1)⁺

### Step G: 2-(5-amino-2-(furan-2-yl)-7H-pyrazolo[4,3-e][1,2,4]triazolo[1,5-c]pyrimidin-7-yl)-2-(o-tolyl)propanoic Acid

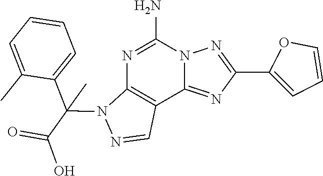 [SMILES: Cc1ccccc1C(C)(C(=O)O)n1ncc2c1nc(N)n1nc(-c3ccco3)nc21]

NaOH solution (24 mg, 0.6 mmol, in 1 mL of water) was added to a solution of ethyl 2-(5-amino-2-(furan-2-yl)-7H-pyrazolo[4,3-e][1,2,4]triazolo[1,5-c]pyrimidin-7-yl)-2-(o-tolyl)propanoate (25 mg, 0.06 mmol) in ethanol (2 mL). The solution was heated at 70° C. for 3 hrs. The solvent was evaporated. The residue was added with water (2 mL), acidified with 2 M HCl to pH=3˜4. The solution was lyophilized to get the crude product, which was used in the next step (20 mg, crude). MS: M/e 404 (M+1)⁺

### Step H: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)-2-(o-tolyl)propan-1-one

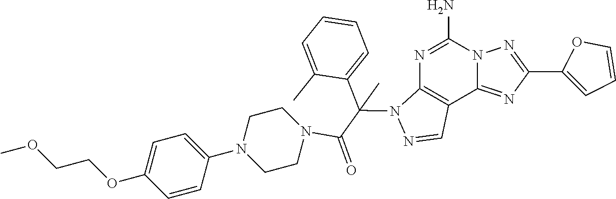 [SMILES: COCCOc1ccc(N2CCN(C(=O)C(C)(c3ccccc3C)n3ncc4c3nc(N)n3nc(-c5ccco5)nc43)CC2)cc1]

A mixture of 2-(5-amino-2-(furan-2-yl)-7H-pyrazolo[4,3-e][1,2,4]triazolo[1,5-c]pyrimidin-7-yl)-2-(o-tolyl)propanoic acid (20 mg, 0.05 mmol), 1-(4-(2-methoxyethoxy)phenyl)piperazine (12 mg, 0.05 mmol), HATU (23 mg, 0.06 mmol) and DIPEA (13 mg, 0.10 mmol) in DMF (5 mL) was stirred at rt for 2 hrs. The solution was added with water (10 mL), extracted with ethyl acetate (10 mL) and washed with brine (10 mL). The organic layer was dried, concentrated and purified by preparative TLC (EA) to get the desired product (5 mg, 17%). ¹H NMR (400 MHz, DMSO-d6) δ 8.30 (s, 1H), 8.11 (br.s, 2H), 8.02 (s, 1H), 7.31-7.18 (m, 4H), 6.94 (d, J=8.0 Hz, 1H), 6.85-6.80 (m, 5H), 4.03 (t, J=4.0 Hz, 2H), 3.90 (br.s, 1H), 3.65 (t, J=4.0 Hz, 2H), 3.60 (br.s, 1H), 3.34 (s, 3H), 3.30-2.80 (m, 6H), 2.52 (s, 3H), 2.36 (s, 3H) ppm. MS: M/e 622 (M+1)⁺

### Compound A104: 4-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)-N,N-dimethylbenzamide

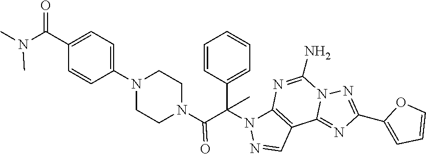 [SMILES: CN(C)C(=O)c1ccc(N2CCN(C(=O)C(C)(c3ccccc3)n3ncc4c3nc(N)n3nc(-c5ccco5)nc43)CC2)cc1]

To a stirred solution of 4-(4-(2-(5-amino-2-(furan-2-yl)-7H-pyrazolo[4,3-e][1,2,4]triazolo[1,5-c]pyrimidin-7-yl)-2-phenylpropanoyl)piperazin-1-yl)benzoic acid (80 mg, 0.14 mmol), HATU (79 mg, 0.21 mmol) and DIPEA (54 mg, 0.42 mmol) in THF (15 ml) was added dimethylamine hydrochloride (17 mg, 0.21 mmol). The reaction mixture was stirred at rt for 15 h. After completion, the reaction mixture was diluted with EA (30 ml) and then washed with H₂O (15 ml×2). The organic layer was dried over Na₂SO₄, filtered and then concentrated under reduced pressure to afford a residue. The residue was purified by prep-TLC with DCM:MeOH (10:1) to afford the product (13.5 mg, 16%). ¹H NMR (400 MHz, DMSO-d6) δ 8.32 (s, 1H), 8.07 (s, 2H), 7.95 (s, 1H), 7.37-7.29 (m, 3H), 7.25-7.18 (m, 5H), 6.79 (d, J=8 Hz, 2H), 6.74 (dd, J=4 Hz, 2 Hz, 1H), 3.95-3.50 (m, 3H), 3.24-2.95 (m, 5H), 2.90 (s, 6H), 2.34 (s, 3H) ppm. MS: M/e 605 (M+1)⁺.

### Compound A105: 2-(5-amino-2-(furan-2-yl)-7H-pyrazolo[4,3-e][1,2,4]triazolo[1,5-c]pyrimidin-7-yl)-1-(4-(2,4-dimethoxyphenyl)piperazin-1-yl)-2-phenylpropan-1-one

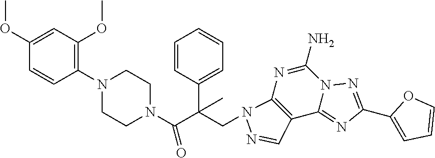 [SMILES: COc1ccc(N2CCN(C(=O)C(C)(Cn3ncc4c3nc(N)n3nc(-c5ccco5)nc43)c3ccccc3)CC2)c(OC)c1]

A mixture of 2-(5-amino-2-(furan-2-yl)-7H-pyrazolo[4,3-e][1,2,4]triazolo[1,5-c]pyrimidin-7-yl)-2-phenylpropanoic acid (30 mg, 0.077 mmol), 1-(2,4-dimethoxyphenyl)piperazine hydrochloride (20 mg, 0.077 mmol), HATU (35.3 mg, 0.093 mmol) and DIPEA (20 mg, 0.134 mmol) in DMF (4 mL) was stirred overnight. The reaction mixture was poured into H₂O (10 mL) and extracted with EtOAc (10 mL×3). The combined organic layers were washed with brine, dried over Na₂SO₄, concentrated and purified by prep-TLC (petroleum ether/EtOAc=1:2) to give the target compound (23 mg, 50.3%). ¹H NMR (400 MHz, DMSO-d6) δ 8.30 (s, 1H), 8.09 (s, 2H), 7.96 (s, 1H), 7.37-7.17 (m, 6H), 6.75 (m, 1H), 6.56 (d, J=8.4 Hz, 1H), 6.45 (d, J=2.4 Hz, 1H), 6.32 (dd, J=8.8, 2.4 Hz, 1H), 3.67 (m, 1H), 3.65 (d, J=4.4 Hz, 6H), 3.31 (s, 1H), 3.16-2.6 (m, 4H), 2.33 (s, 3H), 2.31-2.09 (m, 2H) ppm. MS: M/e 594 (M+1)⁺.

### Compound A106: 2-(5-amino-2-(furan-2-yl)-7H-pyrazolo[4,3-e][1,2,4]triazolo[1,5-c]pyrimidin-7-yl)-1-(4-(2-methoxy-4-(2-methoxyethoxy)phenyl)piperazin-1-yl)-2-phenylpropan-1-one

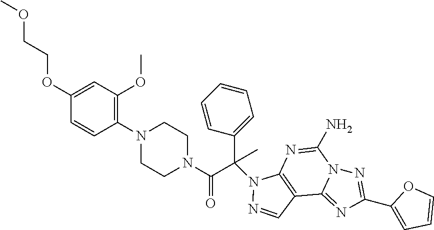 [SMILES: COCCOc1ccc(N2CCN(C(=O)C(C)(c3ccccc3)n3ncc4c3nc(N)n3nc(-c5ccco5)nc43)CC2)c(OC)c1]

A mixture of 2-(5-amino-2-(furan-2-yl)-7H-pyrazolo[4,3-e][1,2,4]triazolo[1,5-c]pyrimidin-7-yl)-2-phenylpropanoic acid (30 mg, 0.077 mmol), 1-(2-methoxy-4-(2-methoxyethoxy)phenyl)piperazine hydrochloride (23.3 mg, 0.077 mmol), HATU (35.3 mg, 0.093 mmol) and DIPEA (20 mg, 0.134 mmol) in DMF (4 mL) was stirred overnight. The reaction mixture was poured into H₂O (10 mL) and extracted with EtOAc (10 mL×3). The combined organic layers were washed with brine, dried over Na₂SO₄, concentrated and purified by prep-TLC (petroleum ether/EtOAc=1:2) to give the target compound (25 mg, 50.9%). ¹H NMR (400 MHz, DMSO-d6) δ 8.30 (s, 1H), 8.09 (s, 2H), 7.96 (s, 1H), 7.38-7.17 (m, 6H), 6.78-6.72 (m, 1H), 6.55 (d, J=8.4 Hz, 1H), 6.46 (s, 1H), 6.33 (d, J=8.4 Hz, 1H), 4.07-3.91 (m, 2H), 3.66 (s, 5H), 3.62-3.57 (m, 2H), 3.27 (s, 3H), 3.12-2.69 (m, 4H), 2.33 (s, 3H), 2.31-2.07 (m, 2H) ppm. MS: M/e 638 (M+1)⁺.

### Compound A107: 2-(5-amino-2-(furan-2-yl)-7H-pyrazolo[4,3-e][1,2,4]triazolo[1,5-c]pyrimidin-7-yl)-1-(4-(4-(2-(methylamino)ethoxy)phenyl)piperazin-1-yl)-2-phenylpropan-1-one

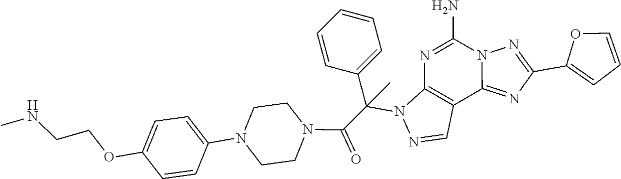 [SMILES: CNCCOc1ccc(N2CCN(C(=O)C(C)(c3ccccc3)n3ncc4c3nc(N)n3nc(-c5ccco5)nc43)CC2)cc1]

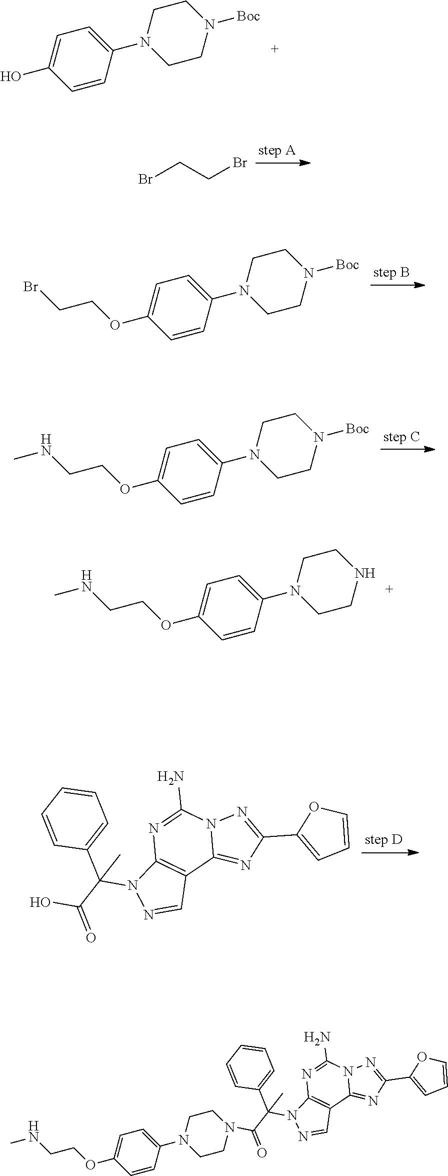

### Step A: tert-butyl 4-(4-(2-bromoethoxy)phenyl)piperazine-1-carboxylate

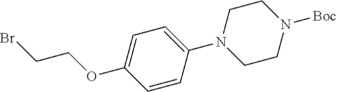 [SMILES: CC(C)(C)OC(=O)N1CCN(c2ccc(OCCBr)cc2)CC1]

To a stirred solution of tert-butyl 4-(4-hydroxyphenyl)piperazine-1-carboxylate (7 g, 25.1 mmol) in acetonitrile (150 mL) was added K₂CO₃ (10 g, 72.4 mmol) and 1,2-dibromoethane (10 g, 53.3 mmol). After the addition, the reaction mixture was stirred for 4 days. The reaction mixture was filtered. The filtrate was poured into H₂O (150 mL) and extracted with EtOAc (100 mL×3). The combined organic layers were washed with brine, dried over Na₂SO₄, concentrated and purified by column chromatography (petroleum ether/EtOAc=4:1) to give tert-butyl 4-(4-(2-bromoethoxy)phenyl)piperazine-1-carboxylate (1.5 g, 15.6%) as white solid. MS: M/e 385 (M+1)⁺.

### Step B: tert-butyl 4-(4-(2-(methylaminoethoxy)phenyl)piperazine-1-carboxylate

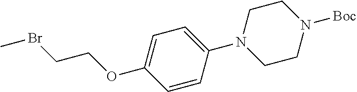

To a mixture of tert-butyl 4-(4-(2-bromoethoxy)phenyl)piperazine-1-carboxylate (218 mg, 0.57 mmol) in MeCN/H₂O (10 mL/1 mL) was added methylamine hydrochloride (330 mg, 5 mmol) and DIEA (774 mg, 6 mmol). After the addition, the reaction mixture was sealed and stirred at 90° C. overnight. The mixture was cooled down to RT H₂O (20 ml) was added to the mixture and the mixture was extracted by DCM (20 mL×3). The combined organic phase was dried over Na₂SO₄, concentrated in vacuo and purified by column chromatography (DCM/MeOH=20:1˜5:1) to give the target compound (130 mg, 68.4%) as a white solid. MS: M/e 336 (M+1)⁺.

### Step C: N-methyl-2-(4-(piperazin-1-yl)phenoxy)ethan-1-amine

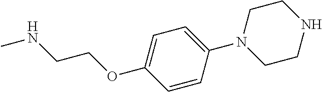 [SMILES: CNCCOc1ccc(N2CCNCC2)cc1]

A mixture of the product of step B (130 mg, 0.39 mmol) in HCl/EA (4M, 10 ml) was stirred at RT for 2 h. The mixture was filtered and the filter cake was washed by EtOAc (10 ml), dried at 40° C. to give the product as a white solid (80 mg, 87.7%). MS: M/e 236 (M+1)⁺.

### Step D: 2-(5-amino-2-(furan-2-yl)-7H-pyrazolo[4,3-e][1,2,4]triazolo[1,5-c]pyrimidin-7-yl)-1-(4-(4-(2-(methylamino)ethoxy)phenyl)piperazin-1-yl)-2-phenylpropan-1-one

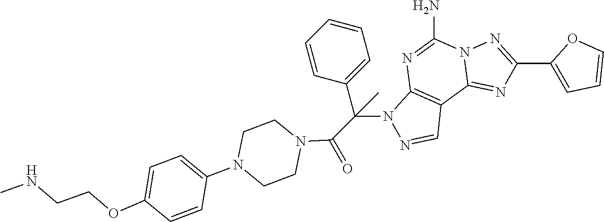 [SMILES: CNCCOc1ccc(N2CCN(C(=O)C(C)(c3ccccc3)n3ncc4c3nc(N)n3nc(-c5ccco5)nc43)CC2)cc1]

A mixture of 2-(5-amino-2-(furan-2-yl)-7H-pyrazolo[4,3-e][1,2,4]triazolo[1,5-c]pyrimidin-7-yl)-2-phenylpropanoic acid (30 mg, 0.077 mmol), HATU (35 mg, 0.092 mmol) and DIPEA (90 mg, 0.697 mmol) in DMF (3 mL) was stirred for 0.5 h at RT. N-methyl-2-(4-(piperazin-1-yl)phenoxy)ethan-1-amine (64 mg, 0.237 mmol) in DMF (2 ml) was added slowly to the mixture over 15 mins. The reaction mixture was stirred at RT for 1 h. The reaction mixture was poured into H₂O (20 mL) and filtered. The filter cake was dried and purified by prep-HPLC to give the target compound (8 mg, 17.1%). ¹H NMR (400 MHz, DMSO-d6) δ 8.53 (s, 1H), 8.32 (s, 1H), 8.09 (s, 2H), 7.95 (s, 1H), 7.26 (m, 6H), 6.79 (m, 5H), 4.08 (m, 2H), 3.70 (m, 4H), 3.27 (s, 3H), 2.97 (s, 4H), 2.61 (s, 2H), 2.33 (s, 3H) ppm. MS: M/e 607.2 (M+1)⁺.

### Compound A108: 2-(5-amino-2-(furan-2-yl)-7H-pyrazolo[4,3-e][1,2,4]triazolo[1,5-c]pyrimidin-7-yl)-1-(4-(4-(2-hydroxyethoxy)phenyl)piperazin-1-yl)-2-phenylpropan-1-one

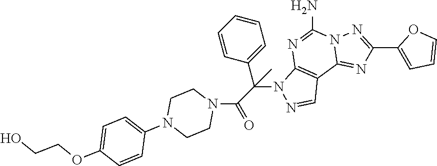 [SMILES: CC(C(=O)N1CCN(c2ccc(OCCO)cc2)CC1)(c1ccccc1)n1ncc2c1nc(N)n1nc(-c3ccco3)nc21]

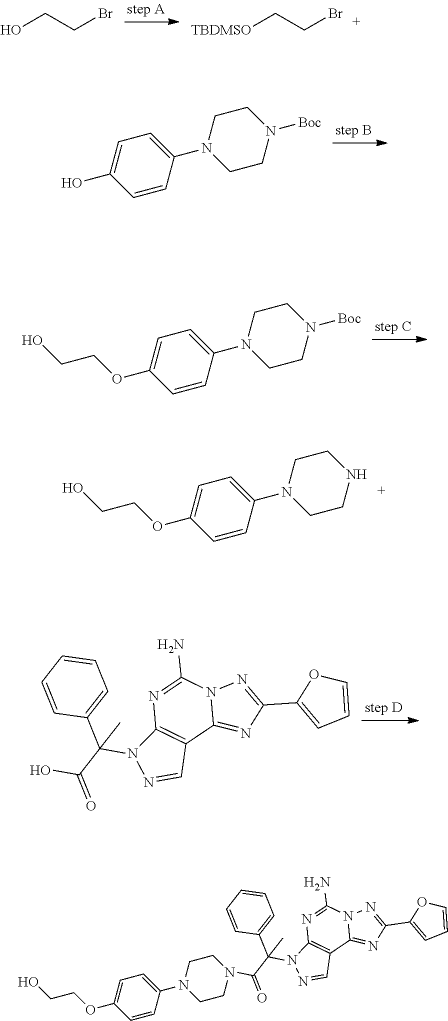

### Step A: (2-bromoethoxy)(tert-butyl)dimethylsilane

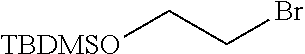 [SMILES: CC(C)(C)[Si](C)(C)OCCBr]

To a stirred mixture of 2-bromoethan-1-ol (8.8 mg, 70.5 mmol) in DCM (50 mL) was added TBDMSCl (10.6 g, 70.5 mmol) and imidazole (9.6 g, 141 mmol). The mixture was stirred at RT overnight. The mixture was added with H₂O (100 ml) and extracted with DCM (50 mL×3). The combined organic phase was washed with brine, dried over Na₂SO₄ and concentrated in vacuo to give the product as a colorless liquid (16 g, 96.3%). ¹H NMR (400 MHz, CDCl₃-d6) δ 3.80 (t, J=6.5 Hz, 2H), 3.30 (t, J=6.5 Hz, 2H), 0.82 (s, 9H). MS: M/e 356 (M+1)⁺.

### Step B: tert-butyl 4-(4-(2-hydroxyethoxy)phenyl)piperazine-1-carboxylate

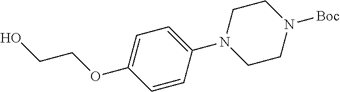 [SMILES: CC(C)(C)OC(=O)N1CCN(c2ccc(OCCO)cc2)CC1]

To a mixture of tert-butyl 4-(4-hydroxyphenyl)piperazine-1-carboxylate (278 mg, 1 mmol) in DMF (5 ml) was added NaH (160 mg, 4 mmol, 60% in oil) at 0° C. The mixture was stirred at RT for 30 mins and (2-bromoethoxy)(tert-butyl)dimethylsilane (476 mg, 2 mmol) was added. The resulting solution was stirred at 70° C. overnight. The mixture was cooled down to 0° C. and H₂O was added. The mixture was extracted by EA (50 mL×3). The combined organic phase was dried over Na₂SO₄, concentrated in vacuo and purified by column chromatography (petroleum ether/EtOAc=2/1˜1/1) to give the target compound (110 mg, 34.2%) as a white solid. MS: M/e 323 (M+1)⁺.

### Step C: 2-(4-(piperazin-1-yl)phenoxy)ethan-1-ol

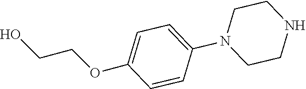 [SMILES: OCCOc1ccc(N2CCNCC2)cc1]

A mixture of tert-butyl 4-(4-(2-hydroxyethoxy)phenyl)piperazine-1-carboxylate (11 mg, 0.34 mmol) in HCl/EA (4M, 10 ml) was stirred at RT for 2 h. The mixture was filtered and the filter cake was washed by EtOAc (10 ml), dried at 40° C. to give the product as a white solid (70 mg, 92.7%). MS: M/e 223 (M+1)⁺.

### Step D: 2-(5-amino-2-(furan-2-yl)-7H-pyrazolo[4,3-e][1,2,4]triazolo[1,5-c]pyrimidin-7-yl)-1-(4-(4-(2-hydroxyethoxy)phenyl)piperazin-1-yl)-2-phenylpropan-1-one

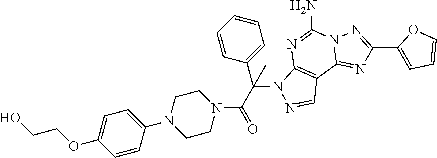 [SMILES: CC(C(=O)N1CCN(c2ccc(OCCO)cc2)CC1)(c1ccccc1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)-2-phenylpropanoic acid (30 mg, 0.077 mmol), 2-(4-(piperazin-1-yl)phenoxy)ethan-1-ol (40 mg, 0.18 mmol), HATU (35 mg, 0.092 mmol) and DIPEA (60 mg, 0.46 mmol) in DMF (5 mL) was stirred for 2 hours at RT. The reaction mixture was poured into H₂O (20 mL) and filtered. The filter cake was dried and purified by prep-HPLC to give the target compound (20 mg, 43.8%). ¹H NMR (400 MHz, DMSO-d6) δ 8.32 (s, 1H), 8.09 (s, 2H), 7.95 (s, 1H), 7.36-7.15 (m, 6H), 6.75 (dd, J=6.7, 4.9 Hz, 5H), 4.53 (s, 4H), 3.85 (t, J=5.0 Hz, 2H), 3.71 (s, 1H), 3.64 (t, J=4.9 Hz, 2H), 2.99 (s, 4H), 2.34 (s, 3H). MS: M/e 594 (M+1)⁺.

### Compound A81a: (S)-2-(5-amino-2-(furan-2-yl)-7H-pyrazolo[4,3-e][1,2,4]triazolo[1,5-c]pyrimidin-7-yl)-1-(4-(4-(2-(4-methylpiperazin-1-yl)ethoxy)phenyl)piperazin-1-yl)-2-phenylpropan-1-one

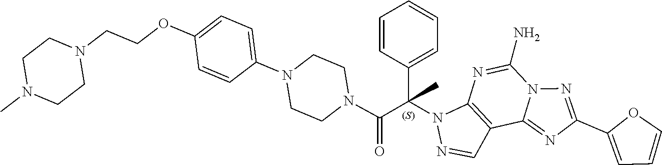

A mixture of (S)-2-(5-amino-2-(furan-2-yl)-7H-pyrazolo[4,3-e][1,2,4]triazolo[1,5-c]pyrimidin-7-yl)-2-phenylpropanoic acid (50 mg, 0.13 mmol), 1-methyl-4-(2-(4-(piperazin-1-yl)phenoxy)ethyl)piperazine hydrochloride (72 mg, 0.16 mmol), HATU (61 mg, 0.16 mmol) and DIEA (50 mg, 0.39 mmol) in DMF (10 mL) was stirred at rt for 2 hrs. The solution was added with water (10 mL), extracted with ethyl acetate (10 mL) and washed with brine (10 mL). The organic layer was dried, concentrated and purified by column chromatography (DCM:MeOH=20:1) and preparative HPLC to get the desired product as a TFA salt (50 mg, 50%). ¹H NMR (400 MHz, DMSO-d6) δ 8.32 (s, 1H), 8.09 (br.s, 2H), 7.95 (s, 1H), 7.31-7.18 (m, 6H), 6.81-6.75 (m, 5H), 4.08 (s, 2H), 3.70-2.97 (m, 18H), 2.80 (s, 3H), 2.33 (s, 3H) ppm. MS: M/e 676 (M+1)⁺

### Compound A81b: (R)-2-(5-amino-2-(furan-2-yl)-7H-pyrazolo[4,3-e][1,2,4]triazolo[1,5-c]pyrimidin-7-yl)-1-(4-(4-(2-(4-methylpiperazin-1-yl)ethoxy)phenyl)piperazin-1-yl)-2-phenylpropan-1-one

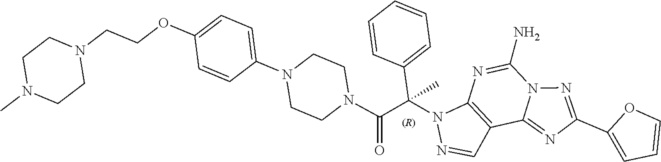

A mixture of (R)-2-(5-amino-2-(furan-2-yl)-7H-pyrazolo[4,3-e][1,2,4]triazolo[1,5-c]pyrimidin-7-yl)-2-phenylpropanoic acid (50 mg, 0.13 mmol), 1-methyl-4-(2-(4-(piperazin-1-yl)phenoxy)ethyl)piperazine hydrochloride (72 mg, 0.16 mmol), HATU (61 mg, 0.16 mmol) and DIEA (50 mg, 0.39 mmol) in DMF (10 mL) was stirred at rt for 2 hrs. The solution was added with water (10 mL), extracted with ethyl acetate (10 mL) and washed with brine (10 mL). The organic layer was dried, concentrated and purified by column chromatography (DCM:MeOH=20:1) and preparative HPLC to get the desired product as a TFA salt (40 mg, 40%). ¹H NMR (400 MHz, DMSO-d6) δ 8.32 (s, 1H), 8.09 (br.s, 2H), 7.95 (s, 1H), 7.33-7.18 (m, 6H), 6.81-6.74 (m, 5H), 4.09 (s, 2H), 3.70-2.97 (m, 18H), 2.80 (s, 3H), 2.33 (s, 3H) ppm. MS: M/e 676 (M+1)⁺

### Compound A82a: (S)-2-(5-amino-2-(furan-2-yl)-7H-pyrazolo[4,3-e][1,2,4]triazolo[1,5-c]pyrimidin-7-yl)-1-(4-(4-(3-(4-methylpiperazin-1-yl)propoxy)phenyl)piperazin-1-yl)-2-phenylpropan-1-one

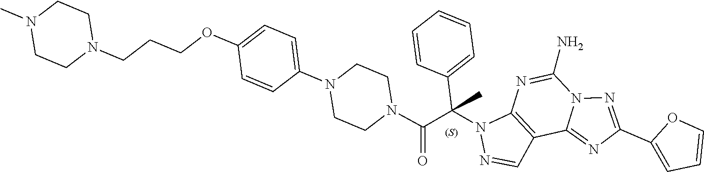

A mixture of (S)-2-(5-amino-2-(furan-2-yl)-7H-pyrazolo[4,3-e][1,2,4]triazolo[1,5-c]pyrimidin-7-yl)-2-phenylpropanoic acid (50 mg, 0.13 mmol), 1-methyl-4-(3-(4-(piperazin-1-yl)phenoxy)propyl)piperazine hydrochloride (72 mg, 0.16 mmol), HATU (61 mg, 0.16 mmol) and DIEA (50 mg, 0.39 mmol) in DMF (5 mL) was stirred at rt for 2 hrs. The solution was added with water (10 mL), extracted with ethyl acetate (10 mL) and washed with brine (10 mL). The organic layer was dried, concentrated and purified by column chromatography (DCM:MeOH=15:1) and preparative HPLC to get the desired product as a TFA salt (45 mg, 44%). ¹H NMR (400 MHz, DMSO-d6) δ 8.31 (s, 1H), 8.08 (br.s, 2H), 7.95 (s, 1H), 7.31-7.18 (m, 6H), 6.75-6.72 (m, 5H), 3.86 (t, J=8.0 Hz, 2H), 3.70 (br.s, 2H), 2.93 (br.s, 4H), 2.74-2.50 (m, 5H), 2.50-2.33 (m, 9H), 1.80 (s, 2H) ppm. MS: M/e 690 (M+1)⁺

### Compound A82b: (R)-2-(5-amino-2-(furan-2-yl)-7H-pyrazolo[4,3-e][1,2,4]triazolo[1,5-c]pyrimidin-7-yl)-1-(4-(4-(3-(4-methylpiperazin-1-yl)propoxy)phenyl)piperazin-1-yl)-2-phenylpropan-1-one

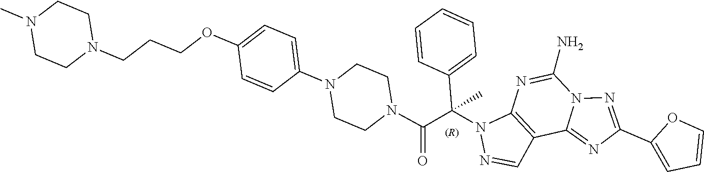

A mixture of (R)-2-(5-amino-2-(furan-2-yl)-7H-pyrazolo[4,3-e][1,2,4]triazolo[1,5-c]pyrimidin-7-yl)-2-phenylpropanoic acid (50 mg, 0.13 mmol), 1-methyl-4-(3-(4-(piperazin-1-yl)phenoxy)propyl)piperazine hydrochloride (72 mg, 0.16 mmol), HATU (61 mg, 0.16 mmol) and DIPEA (50 mg, 0.39 mmol) in DMF (5 mL) was stirred at rt for 2 hrs. The solution was added with water (10 mL), extracted with ethyl acetate (10 mL) and washed with brine (10 mL). The organic layer was dried, concentrated and purified by column chromatography (DCM:MeOH=15:1) and preparative HPLC to get the desired product as a TFA salt (48 mg, 47%). ¹H NMR (400 MHz, DMSO-d6) δ 8.32 (s, 1H), 8.09 (br.s, 2H), 7.95 (d, J=4.0 Hz, 1H), 7.33-7.18 (m, 6H), 6.75-6.74 (m, 5H), 3.91 (t, J=8.0 Hz, 2H), 3.7-2.50 (m, 21H), 2.33 (s, 3H), 1.96 (s, 2H) ppm. MS: M/e 690 (M+1)⁺

### Compound B1: 2-(5-amino-2-(furan-2-yl)-7H-pyrazolo[4,3-e][1,2,4]triazolo[1,5-c]pyrimidin-7-yl)-N,N-dimethyl-2-phenylacetamide

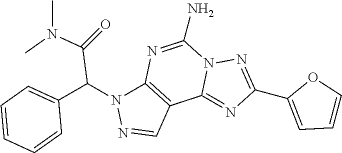 [SMILES: CN(C)C(=O)C(c1ccccc1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)-2-phenylacetic acid (37.5 mg, 0.1 mmol), dimethylamine (2 M, 6.75 mL) and HATU (46 mg, 0.12 mmol) in DMF (2 mL) was stirred overnight. The mixture was poured into H₂O (15 mL) and extracted with EtOAc (10 mL×3). The combined organic layers were washed with brine, dried over Na₂SO₄, concentrated and purified by column chromatography (petroleum ether/EtOAc=2:1˜100% EtOAc) to give the target compound (10 mg, 24.8%). ¹H NMR (400 MHz, DMSO-d6) δ 8.18 (s, 1H), 8.15 (br.s, 1H), 7.95 (s, 1H), 7.35 (m, 5H), 7.24 (d, J=3.2 Hz, 1H), 6.79 (s, 1H), 6.74 (m, 1H), 2.88 (s, 3H), 2.77 (s, 3H) ppm. MS: M/e 403 (M+1)⁺.

### Compound B2: 2-(5-amino-2-(furan-2-yl)-7H-pyrazolo[4,3-e][1,2,4]triazolo[1,5-c]pyrimidin-7-yl)-2-phenyl-1-(piperidin-1-yl)ethan-1-one

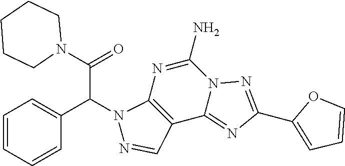 [SMILES: Nc1nc2c(cnn2C(C(=O)N2CCCCC2)c2ccccc2)c2nc(-c3ccco3)nn12]

To a mixture of piperidine (10 mg, 0.12 mmol), 2-(5-amino-2-(furan-2-yl)-7H-pyrazolo[4,3-e][1,2,4]triazolo[1,5-c]pyrimidin-7-yl)-2-phenylacetic acid (30 mg, 0.08 mmol), DIEA (50 mg, 0.39 mmol) in DMF (1 mL) was added HATU (36 mg, 0.09 mmol) at rt and the mixture was stirred at rt for 16 hrs. 5 mL of EA was added and the mixture was washed with NaHCO₃ (3 mL×2), brine (3 mL×2), dried over Na₂SO₄ and concentrated. The resulting residue was purified by prep-TLC (PE/EA=1:2) to give the title product (12.0 mg, yield: 34%). ¹H NMR (400 MHz, DMSO-d6) δ 8.17 (s, 1H), 8.13 (s, 2H), 7.95 (s, 1H), 7.43-7.31 (m, 5H), 7.24 (d, J=3.2 Hz, 1H), 6.78 (s, 1H), 6.75-6.70 (m, 1H), 3.63-3.50 (m, 1H), 3.44-3.33 (m, 2H), 3.27-3.18 (m, 1H), 1.55-1.36 (m, 4H), 1.12-1.00 (m, 1H), 1.00-0.88 (m, 1H). MS: M/e 443 (M+1)⁺.

### Compound B3: 2-(5-amino-2-(furan-2-yl)-7H-pyrazolo[4,3-e][1,2,4]triazolo[1,5-c]pyrimidin-7-yl)-1-(3,4-dihydroisoquinolin-2(1H)-yl)-2-phenylethan-1-one

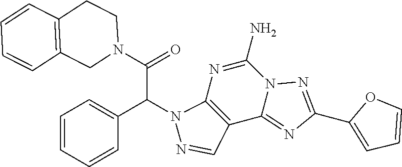 [SMILES: Nc1nc2c(cnn2C(C(=O)N2CCc3ccccc3C2)c2ccccc2)c2nc(-c3ccco3)nn12]

To a mixture of 1,2,3,4-tetrahydroisoquinoline (200 mg, 1.5 mmol), 2-(5-amino-2-(furan-2-yl)-7H-pyrazolo[4,3-e][1,2,4]triazolo[1,5-c]pyrimidin-7-yl)-2-phenylacetic acid (400 mg, 1.07 mmol), DIPEA (620 mg, 4.8 mmol) in DMF (5 mL) was added HATU (460 mg, 1.2 mmol) at rt and the mixture was stirred at rt for 16 hrs. The mixture was poured into 20 mL of H₂O. A white solid precipitated and which was filtered. The solid was washed with H₂O, dried under high vacuum and purified by column chromatograph to give the title product (335.0 mg, yield: 46%). ¹H NMR (400 MHz, DMSO-d6) δ 8.38-8.02 (m, 3H), 7.95 (s, 1H), 7.46-6.92 (m, 11H), 6.74 (s, 1H), 4.75-4.32 (m, 2H), 3.81-3.46 (m, 2H), 2.84-2.63 (m, 1H), 2.42-2.32 (m, 1H). MS: M/e 491 (M+1)⁺.

Compound B3 was separated into two enantiomeric stereoisomers, Compound B3a (earlier peak), and Compound B3b (later peak) by chiral prep-HPLC. The chiral separation conditions are shown below.

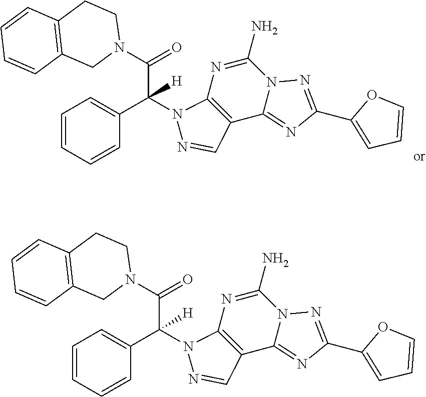 [SMILES: Nc1nc2c(cnn2[C@@H](C(=O)N2CCc3ccccc3C2)c2ccccc2)c2nc(-c3ccco3)nn12 ; Nc1nc2c(cnn2[C@H](C(=O)N2CCc3ccccc3C2)c2ccccc2)c2nc(-c3ccco3)nn12]

| Column | CHIRALPAK IG |
|---|---|
| Column size | 2 cm × 25 cm, 5 um |
| Injection | 1.0 mL |
| Mobile phase | (Hex:DCM = 3:1):EtOH = 50:50 |
| Flow rate | 19 ml/min |
| Wavelength | UV 220 nm |
| Temperature | 25° C. |
| Sample solution | 28.8 mg/ml in DCM:MeOH = 1:3 |
| Prep-HPLC equipment | BJ-Prep-Gilson-HPLC |

### Compound B4: 2-(5-amino-2-(furan-2-yl)-7H-pyrazolo[4,3-e][1,2,4]triazolo[1,5-c]pyrimidin-7-yl)-1-(3,4-dihydroquinolin-1(2H)-yl)-2-phenylethan-1-one

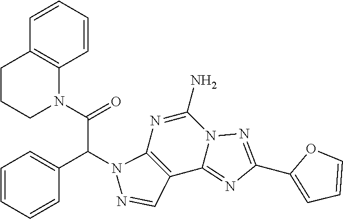 [SMILES: Nc1nc2c(cnn2C(C(=O)N2CCCc3ccccc32)c2ccccc2)c2nc(-c3ccco3)nn12]

To a mixture of 1,2,3,4-tetrahydroquinoline (20 mg, 0.15 mmol), 2-(5-amino-2-(furan-2-yl)-7H-pyrazolo[4,3-e][1,2,4]triazolo[1,5-c]pyrimidin-7-yl)-2-phenylacetic acid (40 mg, 0.11 mmol), DIPEA (100 mg, 0.77 mmol) in DMF (1 mL) was added HATU (50 mg, 0.13 mmol) at rt and the mixture was stirred at rt for 16 hrs. 20 mL of EA was added and the mixture was washed with brine (20 mL×3), dried over Na₂SO₄ and concentrated. The resulting residue was purified by prep-TLC (PE/EA=1:2) to give the title product (21.0 mg, yield: 39%). ¹H NMR (400 MHz, DMSO-d6) δ 8.34-7.99 (m, 3H), 7.95 (s, 1H), 7.72 (d, J=7.6 Hz, 1H), 7.56-7.17 (m, 6H), 7.17-7.07 (m, 2H), 7.04-6.78 (m, 2H), 6.74 (s, 1H), 3.80-3.49 (m, 2H), 1.92-1.60 m, 2H), 1.37-1.16 (m, 2H). MS: M/e 491 (M+1)⁺.

### Compound B5: 2-(5-amino-2-(furan-2-yl)-7H-pyrazolo[4,3-e][1,2,4]triazolo[1,5-c]pyrimidin-7-yl)-1-(7-chloro-3,4-dihydroisoquinolin-2(1H)-yl)-2-phenylethan-1-one

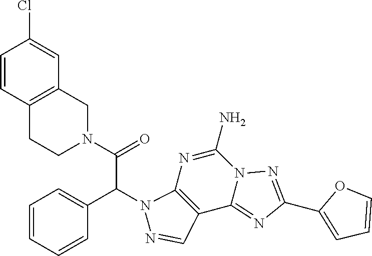 [SMILES: Nc1nc2c(cnn2C(C(=O)N2CCc3ccc(Cl)cc3C2)c2ccccc2)c2nc(-c3ccco3)nn12]

To a mixture of 7-chloro-1,2,3,4-tetrahydroisoquinoline (20 mg, 0.12 mmol), 2-(5-amino-2-(furan-2-yl)-7H-pyrazolo[4,3-e][1,2,4]triazolo[1,5-c]pyrimidin-7-yl)-2-phenylacetic acid (40 mg, 0.11 mmol), DIPEA (100 mg, 0.77 mmol) in DMF (1 mL) was added HATU (50 mg, 0.13 mmol) at rt and the mixture was stirred at rt for 16 hrs. 20 mL of EA was added and the mixture was washed with brine (10 mL×3), dried over Na₂SO₄ and concentrated. The resulting residue was purified by prep-TLC (PE/EA=1:2) to give the title product (16.0 mg, yield: 28%). ¹H NMR (400 MHz, DMSO-d6) δ 8.24-8.03 (m, 3H), 7.95 (s, 1H), 7.50-7.26 (m, 6H), 7.24 (s, 1H), 7.21-7.03 (m, 2H), 6.95-6.88 (m, 1H), 6.74 (s, 1H), 4.78-4.33 (m, 2H), 3.80-3.49 (m, 2H), 2.84-2.27 (m, 2H). MS: M/e 525 (M+1)⁺.

### Compound B6: 2-(5-amino-2-(furan-2-yl)-7H-pyrazolo[4,3-e][1,2,4]triazolo[1,5-c]pyrimidin-7-yl)-2-phenyl-1-(7-(trifluoromethoxy)-3,4-dihydroisoquinolin-2(1H)-yl)ethan-1-one

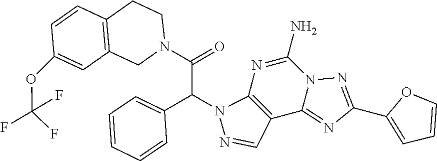 [SMILES: Nc1nc2c(cnn2C(C(=O)N2CCc3ccc(OC(F)(F)F)cc3C2)c2ccccc2)c2nc(-c3ccco3)nn12]

To a mixture of 7-(trifluoromethoxy)-1,2,3,4-tetrahydroisoquinoline (35 mg, 0.16 mmol), 2-(5-amino-2-(furan-2-yl)-7H-pyrazolo[4,3-e][1,2,4]triazolo[1,5-c]pyrimidin-7-yl)-2-phenylacetic acid (50 mg, 0.13 mmol), DIPEA (70 mg, 0.54 mmol) in DMF (2 mL) was added HATU (65 mg, 0.17 mmol) at rt and the mixture was stirred at rt for 16 hrs. 10 mL of EA was added and the mixture was washed with brine (10 mL×3), dried over Na₂SO₄ and concentrated. The resulting residue was purified by prep-TLC (PE/EA=1:2) to give the title product (25.0 mg, yield: 36%). ¹H NMR (400 MHz, DMSO-d6) δ 8.34-8.01 (m, 3H), 7.95 (s, 1H), 7.50-6.59 (m, 11H), 4.85-4.37 (m, 2H), 3.89-3.47 (m, 2H), 2.89-2.29 (m, 2H). MS: M/e 575 (M+1)⁺.

### Compound B7: 2-(5-amino-2-(furan-2-yl)-7H-pyrazolo[4,3-e][1,2,4]triazolo[1,5-c]pyrimidin-7-yl)-1-(6-fluoro-3,4-dihydroisoquinolin-2(1H)-yl)-2-phenylethan-1-one

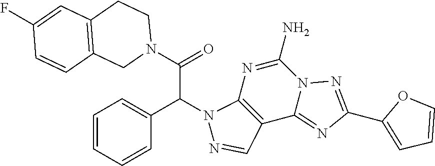 [SMILES: Nc1nc2c(cnn2C(C(=O)N2CCc3cc(F)ccc3C2)c2ccccc2)c2nc(-c3ccco3)nn12]

To a mixture of 6-fluoro-1,2,3,4-tetrahydroisoquinoline (200 mg, 1.32 mmol), 2-(5-amino-2-(furan-2-yl)-7H-pyrazolo[4,3-e][1,2,4]triazolo[1,5-c]pyrimidin-7-yl)-2-phenylacetic acid (400 mg, 1.1 mmol), DIEA (550 mg, 4.3 mmol) in DMF (8 mL) was added HATU (480 mg, 1.3 mmol) at rt and the mixture was stirred at rt for 16 hrs. 50 mL of EA was added and the mixture was washed with brine (20 mL×3), dried over Na₂SO₄ and concentrated. The resulting residue was purified by column chromatograph to give the title product (175.0 mg, yield: 32%). ¹H NMR (400 MHz, DMSO-d6) δ 8.29-8.04 (m, 3H), 7.95 (s, 1H), 7.51-6.69 (m, 11H), 4.81-4.28 (m, 2H), 3.86-3.47 (m, 2H), 2.86-2.71 (m, 1H), 2.41-2.35 (m, 1H). MS: M/e 509 (M+1)⁺.

Compound B7 was separated into two enantiomeric stereoisomers, Compound B7a (earlier peak), and Compound B7b (later peak) by chiral prep-HPLC. The chiral separation conditions are shown below.

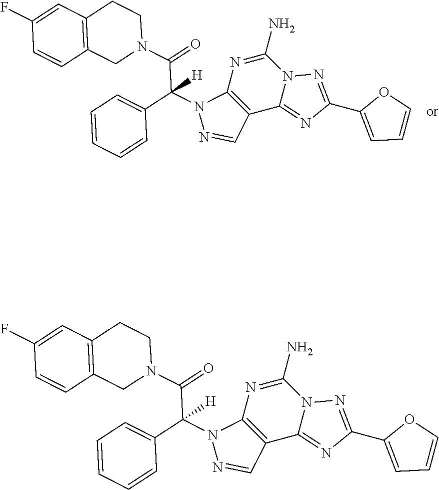 [SMILES: Nc1nc2c(cnn2[C@@H](C(=O)N2CCc3cc(F)ccc3C2)c2ccccc2)c2nc(-c3ccco3)nn12 ; Nc1nc2c(cnn2[C@H](C(=O)N2CCc3cc(F)ccc3C2)c2ccccc2)c2nc(-c3ccco3)nn12]

| Column | Chiralpak IA |
|---|---|
| Column size | 2 cm × 25 cm, 5 um |
| Injection | 1.5 mL |
| Mobile phase | Elex:EtOH = 50:50 |
| Flow rate | 18 ml/min |
| Wavelength | UV 220 nm |
| Temperature | 25° C. |
| Sample solution | 9.7 mg/ml in EtOH:DCM = 1:3 |
| Prep-HPLC equipment | Prep-Gilson-HPLC |

### Compound B8: 2-(5-amino-2-(furan-2-yl)-7H-pyrazolo[4,3-e][1,2,4]triazolo[1,5-c]pyrimidin-7-yl)-1-(3,4-dihydro-2,7-naphthyridin-2(1H)-yl)-2-phenylethan-1-one

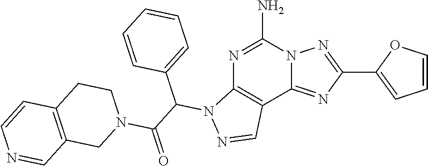 [SMILES: Nc1nc2c(cnn2C(C(=O)N2CCc3ccncc3C2)c2ccccc2)c2nc(-c3ccco3)nn12]

A mixture of 2-(5-amino-2-(furan-2-yl)-7H-pyrazolo[4,3-e][1,2,4]triazolo[1,5-c]pyrimidin-7-yl)-2-phenylacetic acid (50 mg, 0.13 mmol), 1,2,3,4-tetrahydro-2,7-naphthyridine (21.4 mg, 0.16 mmol), HATU (60.8 mg, 0.16 mmol) and DIEA (0.1 mL) in DMF (2 mL) was stirred at RT overnight. The reaction was diluted with water (5 mL) and extracted with EA (10 mL×2). The combined organic phase was washed with brine, dried over Na₂SO₄ and concentrated under reduced pressure. The residue was purified by silica gel chromatography (EA:100%) to afford the title compound (9 mg, yield: 14%). ¹H NMR (400 MHz, DMSO-d6) δ 8.42 (s, 1H), 8.28 (d, J=4 Hz, 1H), 8.22-8.04 (m, 3H), 8.00-7.83 (m, 1H), 7.46-7.20 (m, 6H), 7.08 (d, J=4 Hz, 1H), 6.95 (s, 1H), 6.74 (s, 1H), 4.84-4.61 (m, 1H), 4.55-4.33 (m, 1H), 3.83-3.70 (m, 1H), 3.64-3.55 (m, 1H), 2.87-2.74 (m, 1H), 2.40-2.27 (m, 1H) ppm. MS: M/e 492 (M+1)⁺.

### Compound B9: 2-(5-amino-2-(furan-2-yl)-7H-pyrazolo[4,3-e][1,2,4]triazolo[1,5-c]pyrimidin-7-yl)-1-(5,8-dihydro-1,7-naphthyridin-7(6H)-yl)-2-phenylethan-1-one

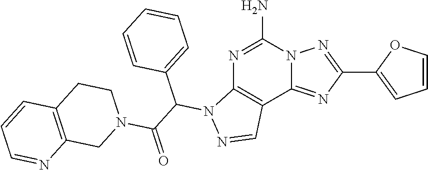 [SMILES: Nc1nc2c(cnn2C(C(=O)N2CCc3cccnc3C2)c2ccccc2)c2nc(-c3ccco3)nn12]

A mixture of 2-(5-amino-2-(furan-2-yl)-7H-pyrazolo[4,3-e][1,2,4]triazolo[1,5-c]pyrimidin-7-yl)-2-phenylacetic acid (50 mg, 0.13 mmol), 5,6,7,8-tetrahydro-1,7-naphthyridine (21.4 mg, 0.16 mmol), HATU (60.8 mg, 0.16 mmol) and DIEA (0.1 mL) in DMF (2 mL) was stirred at RT overnight. The reaction was diluted with water (5 mL) and extracted with EA (10 mL×2). The combined organic phase was washed with brine, dried over Na₂SO₄ and concentrated under reduced pressure. The residue was purified by silica gel chromatography (EA:100%) to afford the title compound (10 mg, yield: 15.6%). ¹H NMR (400 MHz, DMSO-d6) δ 8.41-8.32 (m, 1H), 8.23-8.06 (m, 3H), 7.95 (s, 1H), 7.55-7.28 (m, 6H), 7.24 (d, 1H), 7.21-7.05 (m, 1H), 7.01-6.91 (m, 1H), 6.74 (s, 1H), 4.78-4.62 (m, 1H), 4.54-4.24 (m, 1H), 3.87-3.71 (m, 1H), 3.70-3.55 (m, 1H), 2.88-2.73 (m, 1H), 2.41-2.30 (m, 1H) ppm. MS: M/e 492 (M+1)⁺.

### Compound B10: 2-(5-amino-2-(furan-2-yl)-7H-pyrazolo[4,3-e][1,2,4]triazolo[1,5-c]pyrimidin-7-yl)-1-(8-fluoro-3,4-dihydroisoquinolin-2(1H)-yl)-2-phenylethan-1-one

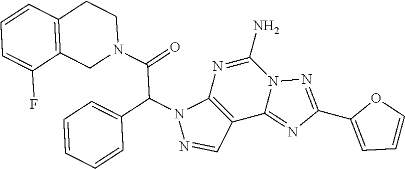 [SMILES: Nc1nc2c(cnn2C(C(=O)N2CCc3cccc(F)c3C2)c2ccccc2)c2nc(-c3ccco3)nn12]

A mixture of 2-(5-amino-2-(furan-2-yl)-7H-pyrazolo[4,3-e][1,2,4]triazolo[1,5-c]pyrimidin-7-yl)-2-phenylacetic acid (50 mg, 0.13 mmol), 8-fluoro-1,2,3,4-tetrahydroisoquinoline hydrochloride (30 mg, 0.16 mmol), HATU (61 mg, 0.16 mmol) and DIEA (34 mg, 0.26 mmol) in DMF (10 mL) was stirred at rt for 2 hrs. The solution was added with water (10 mL), extracted with ethyl acetate (10 mL) and washed with brine (10 mL). The organic layer was dried, concentrated and purified by column chromatography (PE:EA=1:1) to get the product (15 mg, 23%). ¹H NMR (400 MHz, DMSO-d6) δ 8.19-8.05 (m, 2H), 7.95 (s, 1H), 7.44-7.17 (m, 7H), 7.08-6.90 (m, 3H), 6.74-6.71 (m, 1H), 4.73-4.29 (m, 2H), 3.82-3.54 (m, 1H), 2.35 (br.s, 2H) ppm. MS: M/e 509 (M+1)⁺

### Compound B11: 2-(5-amino-2-(furan-2-yl)-7H-pyrazolo[4,3-e][1,2,4]triazolo[1,5-c]pyrimidin-7-yl)-1-(7-fluoro-3,4-dihydroisoquinolin-2(1H)-yl)-2-phenylethan-1-one

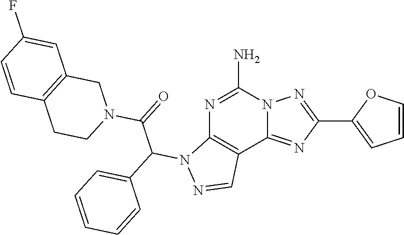 [SMILES: Nc1nc2c(cnn2C(C(=O)N2CCc3ccc(F)cc3C2)c2ccccc2)c2nc(-c3ccco3)nn12]

A mixture of 2-(5-amino-2-(furan-2-yl)-7H-pyrazolo[4,3-e][1,2,4]triazolo[1,5-c]pyrimidin-7-yl)-2-phenylacetic acid (50 mg, 0.13 mmol), 7-fluoro-1,2,3,4-tetrahydroisoquinoline hydrochloride (30 mg, 0.16 mmol), HATU (61 mg, 0.16 mmol) and DIEA (34 mg, 0.26 mmol) in DMF (10 mL) was stirred at rt for 2 hrs. The solution was added with water (10 mL), extracted with ethyl acetate (10 mL) and washed with brine (10 mL). The organic layer was dried, concentrated and purified by column chromatography (PE:EA=1:1) to get the product (15 mg, 23%). ¹H NMR (400 MHz, DMSO-d6) δ 8.19-8.09 (m, 2H), 7.95 (s, 1H), 7.36-7.24 (m, 6H), 7.11-6.90 (m, 4H), 6.74 (br.s, 1H), 4.74-4.39 (m, 2H), 3.76-3.49 (m, 2H), 2.98-2.75 (m, 2H), 2.33 (s, 1H) ppm. MS: M/e 509 (M+1)⁺

### Compound B12: 2-(5-amino-2-(furan-2-yl)-7H-pyrazolo[4,3-e][1,2,4]triazolo[1,5-c]pyrimidin-7-yl)-1-(5-fluoro-3,4-dihydroisoquinolin-2(1H)-yl)-2-phenylethan-1-one

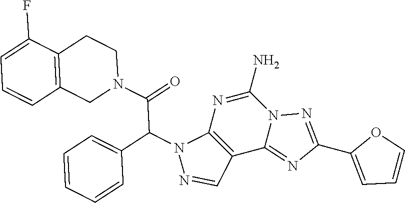 [SMILES: Nc1nc2c(cnn2C(C(=O)N2CCc3c(F)cccc3C2)c2ccccc2)c2nc(-c3ccco3)nn12]

To a mixture of 5-fluoro-1,2,3,4-tetrahydroisoquinoline hydrochloride (30 mg, 0.16 mmol), 2-(5-amino-2-(furan-2-yl)-7H-pyrazolo[4,3-e][1,2,4]triazolo[1,5-c]pyrimidin-7-yl)-2-phenylacetic acid (50 mg, 0.13 mmol), DIEA (70 mg, 0.54 mmol) in DMF (2 mL) was added HATU (65 mg, 0.17 mmol) at rt and the mixture was stirred at rt for 16 hrs. 20 mL of EA was added and the mixture was washed with brine (10 mL×3), dried over Na₂SO₄ and concentrated. The resulting residue was purified by prep-TLC (PE/EA=1:2) to give the title product (26.0 mg, yield: 39%). ¹H NMR (400 MHz, DMSO-d6) δ 8.34-7.89 (m, 4H), 7.49-7.04 (m, 7H), 7.04-6.48 (m, 4H), 4.81-4.34 (m, 2H), 3.84-3.51 (m, 2H), 2.88-2.21 (m, 2H). MS: M/e 509 (M+1)⁺.

### Compound B13: 2-(5-amino-2-(furan-2-yl)-7H-pyrazolo[4,3-e][1,2,4]triazolo[1,5-c]pyrimidin-7-yl)-1-(7,8-dihydro-1,6-naphthyridin-6(5H)-yl)-2-phenylethan-1-one

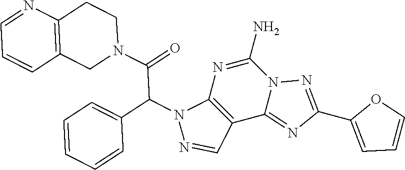 [SMILES: Nc1nc2c(cnn2C(C(=O)N2CCc3ncccc3C2)c2ccccc2)c2nc(-c3ccco3)nn12]

To a mixture of 5,6,7,8-tetrahydro-1,6-naphthyridine (23 mg, 0.17 mmol), 2-(5-amino-2-(furan-2-yl)-7H-pyrazolo[4,3-e][1,2,4]triazolo[1,5-c]pyrimidin-7-yl)-2-phenylacetic acid (50 mg, 0.13 mmol), DIEA (70 mg, 0.54 mmol) in DMF (2 mL) was added HATU (65 mg, 0.17 mmol) at rt and the mixture was stirred at rt for 16 hrs. 20 mL of EA was added and the mixture was washed with brine (10 mL×3), dried over Na₂SO₄ and concentrated. The resulting residue was purified by prep-TLC (EA/MeOH=20:1) to give the title product (28.0 mg, yield: 44%). ¹H NMR (400 MHz, DMSO-d6) δ 8.40-8.23 (m, 1H), 8.24-7.90 (m, 4H), 7.69-6.70 (m, 10H), 4.81-4.35 (m, 2H), 4.00-3.56 (m, 2H), 2.98-2.35 (m, 2H). MS: M/e 492 (M+1)⁺.

### Compound B14: 2-(5-amino-2-(furan-2-yl)-7H-pyrazolo[4,3-e][1,2,4]triazolo[1,5-c]pyrimidin-7-yl)-1-(7-(2-methoxyethoxy)-3,4-dihydroisoquinolin-2(1H)-yl)-2-phenylethan-1-one

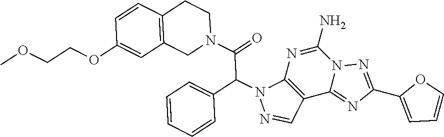 [SMILES: COCCOc1ccc2c(c1)CN(C(=O)C(c1ccccc1)n1ncc3c1nc(N)n1nc(-c4ccco4)nc31)CC2]

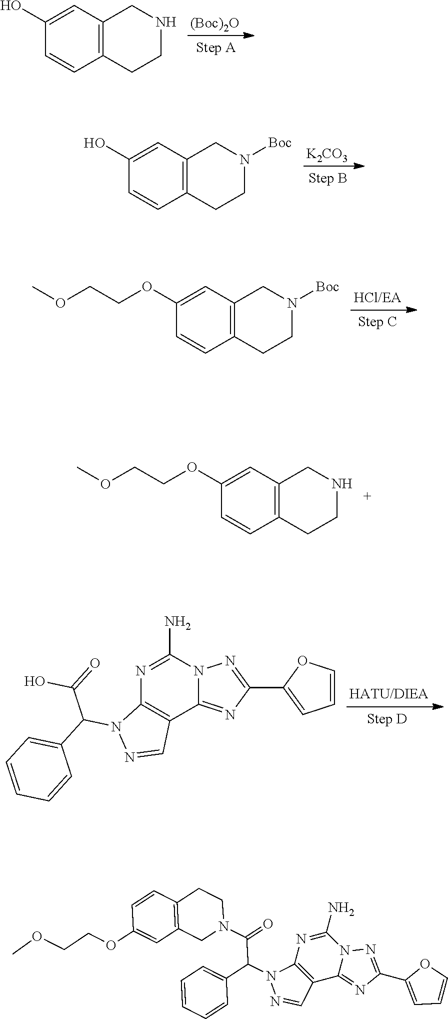

### Step A: tert-butyl 7-hydroxy-3,4-dihydroisoguinoline-2(1H)-carboxylate

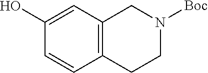 [SMILES: CC(C)(C)OC(=O)N1CCc2ccc(O)cc2C1]

A solution of 1,2,3,4-tetrahydroisoquinolin-7-ol (300 mg, 2 mmol), (Boc)₂O (480 mg, 2.2 mmol) and triethylamine (404 mg, 4 mmol) in DCM (10 mL) was stirred at rt overnight. The reaction mixture was washed with brine (10 mL). The organic layer was dried, concentrated to give the crude product, which was used in the next step directly (500 mg, 100%). MS: M/e 250 (M+1)⁺

### Step B: tert-butyl 7-(2-methoxyethoxy)-3,4-dihydroisoquinoline-2(1H)-carboxylate

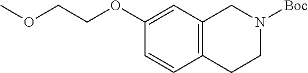 [SMILES: COCCOc1ccc2c(c1)CN(C(=O)OC(C)(C)C)CC2]

A solution of tert-butyl 7-hydroxy-3,4-dihydroisoquinoline-2(1H)-carboxylate (500 mg, 2 mmol), 1-bromo-2-methoxyethane (558 mg, 4 mmol) and K₂CO₃ (552 mg, 4 mmol) in DMF (10 mL) was stirred at rt overnight. The reaction mixture was added with water (10 mL), extracted with EA (10 mL), and washed with brine (10 mL). The organic layer was dried, concentrated to give the crude product, which was purified by column chromatography (PE:EA=10:1) to get the product (160 mg, 26%). MS: M/e 308 (M+1)⁺

### Step C: 7-(2-methoxyethoxy)-1,2,3,4-tetrahydroisoquinoline Hydrochloride

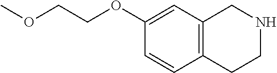

To a solution of tert-butyl 7-(2-methoxyethoxy)-3,4-dihydroisoquinoline-2(1H)-carboxylate (60 mg, 0.2 mmol) in EA (2 mL) was added with HCl/EA (4M, 2 mL). The mixture was stirred at rt for 2 hrs, then evaporated under reduced pressure and used in the next step directly.

### Step D: 2-(5-amino-2-(furan-2-yl)-7H-pyrazolo[4,3-e][1,2,4]triazolo[1,5-c]pyrimidin-7-yl)-1-(7-(2-methoxyethoxy)-3,4-dihydroisoquinolin-2(1H)-yl)-2-phenylethan-1-one

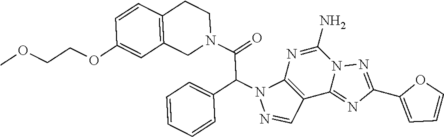 [SMILES: COCCOc1ccc2c(c1)CN(C(=O)C(c1ccccc1)n1ncc3c1nc(N)n1nc(-c4ccco4)nc31)CC2]

A mixture of 2-(5-amino-2-(furan-2-yl)-7H-pyrazolo[4,3-e][1,2,4]triazolo[1,5-c]pyrimidin-7-yl)-2-phenylacetic acid (72 mg, 0.2 mmol), 7-(2-methoxyethoxy)-1,2,3,4-tetrahydroisoquinoline hydrochloride (40 mg, 0.2 mmol), HATU (114 mg, 0.3 mmol) and DIEA (52 mg, 0.4 mmol) in DMF (10 mL) was stirred at rt for 2 hrs. The solution was added with water (10 mL), extracted with ethyl acetate (10 mL) and washed with brine (10 mL). The organic layer was dried, concentrated and purified by column chromatography (PE:EA=1:1) to get the product (30 mg, 28%). ¹H NMR (400 MHz, DMSO-d6) δ 8.19 (s, 1H), 8.17 (br.s, 2H), 7.95 (s, 1H), 7.39-7.24 (m, 6H), 6.93-6.91 (m, 2H), 6.80 (s, 1H), 6.74-6.62 (m, 2H), 4.69-4.59 (m, 1H), 4.38 (s, 1H), 4.04 (t, J=4.0 Hz, 1H), 3.83-3.75 (m, 1H), 3.68-3.46 (m, 4H), 3.29 (s, 2H), 3.22 (s, 1H), 2.72-2.68 (m, 1H), 2.27 (s, 1H) ppm. MS: M/e 565 (M+1)⁺

### Compound B15: 2-(5-amino-2-(furan-2-yl)-7H-pyrazolo[4,3-e][1,2,4]triazolo[1,5-c]pyrimidin-7-yl)-1-(6-(2-methoxyethoxy)-3,4-dihydroisoquinolin-2(1H)-yl)-2-phenylethan-1-one

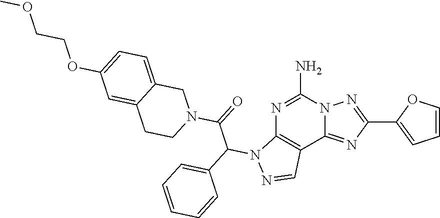 [SMILES: COCCOc1ccc2c(c1)CCN(C(=O)C(c1ccccc1)n1ncc3c1nc(N)n1nc(-c4ccco4)nc31)C2]

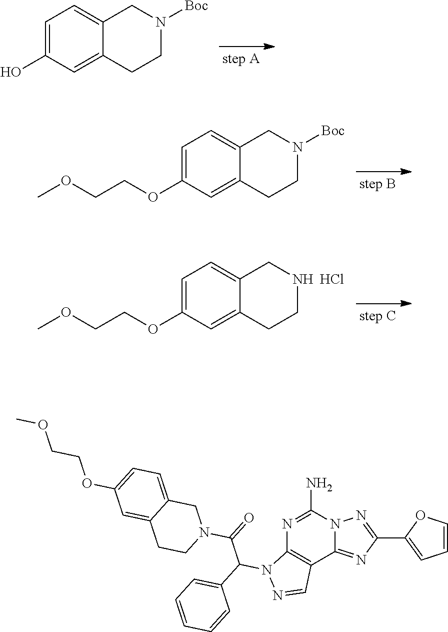 [SMILES: CC(C)(C)OC(=O)N1CCc2cc(O)ccc2C1 ; COCCOc1ccc2c(c1)CCN(C(=O)OC(C)(C)C)C2 ; COCCOc1ccc2c(c1)CCNC2 ; Cl ; COCCOc1ccc2c(c1)CCN(C(=O)C(c1ccccc1)n1ncc3c1nc(N)n1nc(-c4ccco4)nc31)C2]

### Step A: tert-butyl 6-(2-methoxyethoxy)-3,4-dihydroisoquinoline-2(1H)-carboxylate

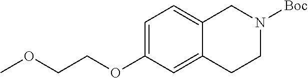 [SMILES: COCCOc1ccc2c(c1)CCN(C(=O)OC(C)(C)C)C2]

To a stirred solution of tert-butyl 6-hydroxy-3,4-dihydroisoquinoline-2(1H)-carboxylate (500 mg, 2 mmol) in DMF (4 mL) was added K₂CO₃ (552 mg, 4 mmol) and 1-bromo-2-methoxyethane (305.8 mg, 2.2 mmol). After the addition, the reaction mixture was stirred for 2 days. The reaction mixture was poured into H₂O (20 mL) and extracted with EtOAc (15 mL×3). The combined organic layers were washed with brine, dried over Na₂SO₄, concentrated to give the target compound (0.62 g, 100%) as brown oil. MS: M/e 308 (M+1)⁺.

### Step B: 6-(2-methoxyethoxy)-1,2,3,4-tetrahydroisoquinoline Hydrochloride

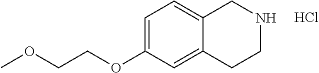

To a stirred solution of the product of step A (0.62 g, 2 mmol) in CH₂Cl₂ (10 mL) was added EtOAc/HCl (4.0 M, 4 mL). After the addition, the reaction mixture was stirred overnight. The reaction mixture was filtered and the cake was collected, dried to give the target compound (400 mg, 82.1%) as a white solid. MS: M/e 208 (M+1)⁺.

### Step C: 2-(5-amino-2-(furan-2-yl)-7H-pyrazolo[4,3-e][1,2,4]triazolo[1,5-c]pyrimidin-7-yl)-1-(6-(2-methoxyethoxy)-3,4-dihydroisoquinolin-2(1H)-yl)-2-phenylethan-1-one

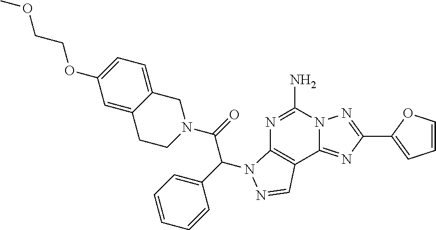 [SMILES: COCCOc1ccc2c(c1)CCN(C(=O)C(c1ccccc1)n1ncc3c1nc(N)n1nc(-c4ccco4)nc31)C2]

A mixture of 2-(5-amino-2-(furan-2-yl)-7H-pyrazolo[4,3-e][1,2,4]triazolo[1,5-c]pyrimidin-7-yl)-2-phenylacetic acid (50 mg, 0.133 mol), the product of step B (27.6 mg, 0.133 mmol), HATU (60 mg, 0.16 mmol) and DIPEA (34 mg, 0.266 mmol) in DMF (4 mL) was stirred overnight. The reaction mixture was poured into H₂O (20 mL) and extracted with EtOAc (15 mL×3). The combined organic layers were washed with brine, dried over Na₂SO₄, concentrated and purified by prep-HPLC to give the target compound (15 mg, 20%). ¹H NMR (400 MHz, DMSO-d6) δ 8.24-8.07 (m, 3H), 7.95 (s, 1H), 7.42-7.31 (m, 5H), 7.25-7.24 (m, 1H), 7.14-6.48 (m, 5H), 4.69-4.27 (m, 2H), 4.02-3.85 (m, 1H), 3.81-3.47 (m, 4H), 3.27 (d, J=6.6 Hz, 3H), 2.86-2.64 (m, 1H), 2.34 (m, 1H) ppm. MS: M/e 565 (M+1)⁺.

### Compound B16: 2-(5-amino-2-(furan-2-yl)-7H-pyrazolo[4,3-e][1,2,4]triazolo[1,5-c]pyrimidin-7-yl)-1-(8-(2-methoxyethoxy)-3,4-dihydroisoquinolin-2(1H)-yl)-2-phenylethan-1-one

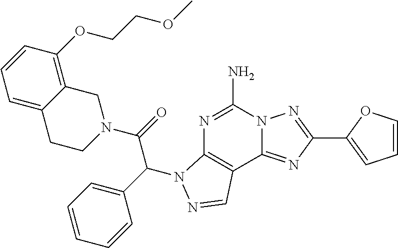 [SMILES: COCCOc1cccc2c1CN(C(=O)C(c1ccccc1)n1ncc3c1nc(N)n1nc(-c4ccco4)nc31)CC2]

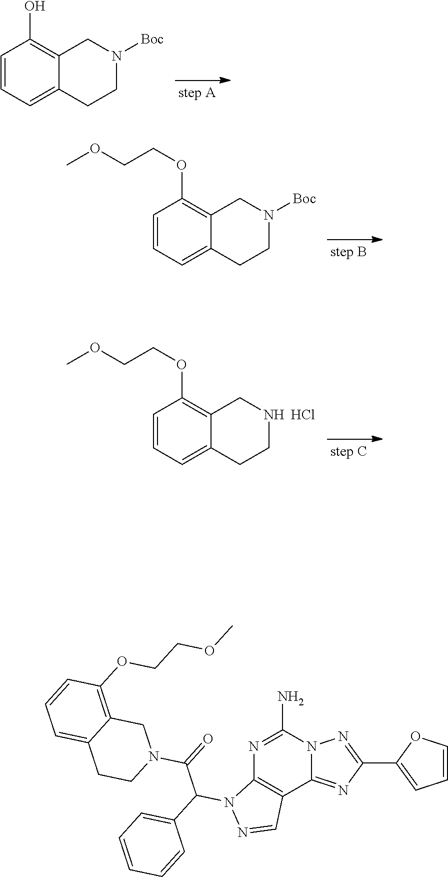

### Step A: tert-butyl 8-(2-methoxyethoxy)-3,4-dihydroisoquinoline-2(1H)-carboxylate

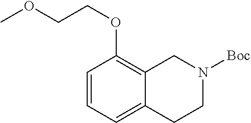 [SMILES: COCCOc1cccc2c1CN(C(=O)OC(C)(C)C)CC2]

To a stirred solution of tert-butyl 8-hydroxy-3,4-dihydroisoquinoline-2(1H)-carboxylate (500 mg, 2 mmol) in DMF (4 mL) was added K₂CO₃ (552 mg, 4 mmol) and 1-bromo-2-methoxyethane (305.8 mg, 2.2 mmol). After the addition, the reaction mixture was stirred for 2 days. The reaction mixture was poured into H₂O (20 mL) and extracted with EtOAc (15 mL×3). The combined organic layers were washed with brine, dried over Na₂SO₄, concentrated to give the target compound (0.63 g, 100%) as brown oil. MS: M/e 308 (M+1)⁺.

### Step B: 8-(2-methoxyethoxy)-1,2,3,4-tetrahydroisoquinoline Hydrochloride

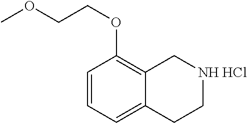

To a stirred solution of the product of step A (0.63 g, 2 mmol) in CH₂Cl₂ (10 mL) was added EtOAc/HCl (4.0 M, 4 mL). After the addition, the reaction mixture was stirred overnight. The reaction mixture was filtered and the cake was collected, dried to give the target compound (350 mg, 71.8%) as a white solid. MS: M/e 208 (M+1)⁺.

### Step C: 2-(5-amino-2-(furan-2-yl)-7H-pyrazolo[4,3-e][1,2,4]triazolo[1,5-c]pyrimidin-7-yl)-1-(8-(2-methoxyethoxy)-3,4-dihydroisoquinolin-2(1H)-yl)-2-phenylethan-1-one

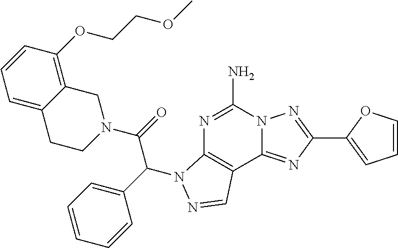 [SMILES: COCCOc1cccc2c1CN(C(=O)C(c1ccccc1)n1ncc3c1nc(N)n1nc(-c4ccco4)nc31)CC2]

A mixture of 2-(5-amino-2-(furan-2-yl)-7H-pyrazolo[4,3-e][1,2,4]triazolo[1,5-c]pyrimidin-7-yl)-2-phenylacetic acid (50 mg, 0.133 mol), the product of step B (27.6 mg, 0.133 mmol), HATU (60 mg, 0.16 mmol) and DIPEA (34 mg, 0.266 mmol) in DMF (4 mL) was stirred overnight. The reaction mixture was poured into H₂O (20 mL) and extracted with EtOAc (15 mL×3). The combined organic layers were washed with brine, dried over Na₂SO₄, concentrated and purified by prep-HPLC to give the target compound (20 mg, 26.7%) as a white solid. ¹H NMR (400 MHz, DMSO-d6) δ 8.24-8.05 (m, 3H), 7.95 (s, 1H), 7.50-7.28 (m, 5H), 7.24 (d, J=2.8 Hz, 1H), 7.17-6.28 (m, 5H), 4.70-4.34 (m, 2H), 4.03 (m, 2H), 3.78-3.49 (m, 4H), 3.28-3.24 (d, J=32.4 Hz, 3H), 2.70-2.60 (m, 1H), 2.21 (m, 1H) ppm. MS: Me 565 (M+1)⁺.

### Compound B17: 2-(5-amino-2-(furan-2-yl)-7H-pyrazolo[4,3-e][1,2,4]triazolo[1,5-c]pyrimidin-7-yl)-1-(7-methyl-3,4-dihydroisoquinolin-2(1H)-yl)-2-phenylethan-1-one

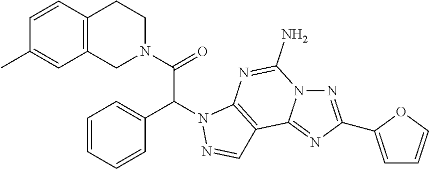 [SMILES: Cc1ccc2c(c1)CN(C(=O)C(c1ccccc1)n1ncc3c1nc(N)n1nc(-c4ccco4)nc31)CC2]

To a mixture of 7-methyl-1,2,3,4-tetrahydroisoquinoline (26 mg, 0.17 mmol), 2-(5-amino-2-(furan-2-yl)-7H-pyrazolo[4,3-e][1,2,4]triazolo[1,5-c]pyrimidin-7-yl)-2-phenylacetic acid (50 mg, 0.13 mmol), DIPEA (80 mg, 0.62 mmol) in DMF (2 mL) was added HATU (65 mg, 0.17 mmol) at rt and the mixture was stirred at rt for 16 hrs. 20 mL of EA was added and the mixture was washed with brine (10 mL×3), dried over Na₂SO₄ and concentrated. The resulting residue was purified by prep-TLC (PE/EA=1:2) to give the title product (25.0 mg, yield: 28%). ¹H NMR (400 MHz, DMSO-d6, 80° C.) δ 8.27-7.96 (m, 1H), 7.97-7.73 (m, 3H), 7.46-7.37 (m, 2H), 7.37-7.25 (m, 3H), 7.20 (d, J=3.2 Hz, 1H), 7.07-6.75 (m, 4H), 6.74-6.66 (m, 1H), 4.75-4.28 (m, 2H), 3.83-3.48 (m, 2H), 2.94-2.59 (m, 2H), 2.32-2.00 (m, 3H). MS: M/e 505 (M+1)⁺.

### Compound B18: 2-(5-amino-2-(furan-2-yl)-7H-pyrazolo[4,3-e][1,2,4]triazolo[1,5-c]pyrimidin-7-yl)-1-(3,4-dihydroisoquinolin-2(1H)-yl)-2-phenylpropan-1-one

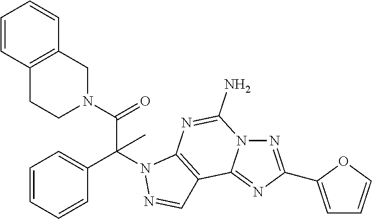 [SMILES: CC(C(=O)N1CCc2ccccc2C1)(c1ccccc1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)-2-phenylpropanoic acid (50 mg, 0.129 mmol), 1,2,3,4-tetrahydroisoquinoline (17 mg, 0.129 mmol), HATU (50 mg, 0.129 mmol) and DIPEA (33 mg, 0.258 mmol) in DMF (5 mL) was stirred for 2 hours at RT. The reaction mixture was poured into H₂O (20 mL) and extracted with EA (20 mL×3). The combined organic layers were washed with brine, dried over Na₂SO₄, concentrated and purified by column chromatography (petroleum ether/EA=1:2) to give the target compound (37 mg, 56.9%). ¹H NMR (400 MHz, DMSO-d6) δ 8.26 (s, 1H), 8.03-7.83 (m, 3H), 7.27-7.21 (m, 6H), 6.96 (s, 1H), 6.82 (s, 1H), 6.73 (s, 1H), 6.61-6.48 (m, 1H), 6.20 (s, 1H), 4.75-4.67 (m, 1H), 4.00 (d, J=16.0 Hz, 1H), 3.76 (d, J=16.0 Hz, 1H), 3.55 (s, 1H), 2.89 (s, 1H), 2.70 (d, J=17.7 Hz, 1H), 2.31 (s, 3H) ppm. MS: M/e 505 (M+1)⁺.

### Compound B19: 2-(5-amino-2-(furan-2-yl)-7H-pyrazolo[4,3-e][1,2,4]triazolo[1,5-c]pyrimidin-7-yl)-1-(6-fluoro-3,4-dihydroisoquinolin-2(1H)-yl)-2-phenylpropan-1-one

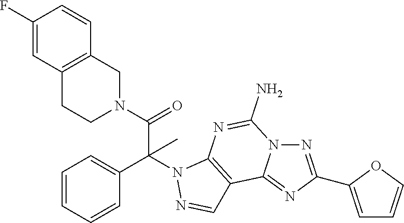 [SMILES: CC(C(=O)N1CCc2cc(F)ccc2C1)(c1ccccc1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)-2-phenylpropanoic acid (50 mg, 0.129 mmol), 6-fluoro-1,2,3,4-tetrahydroisoquinoline (24.2 mg, 0.129 mmol), HATU (50 mg, 0.129 mmol) and DIPEA (33 mg, 0.258 mmol) in DMF (5 mL) was stirred for 2 hours at RT. The reaction mixture was poured into H₂O (20 mL) and extracted with EA (20 mL×3). The combined organic layers were washed with brine, dried over Na₂SO₄, concentrated and purified by column chromatography (petroleum ether/EA=1:2) to give the target compound (28 mg, 41.5%). ¹H NMR (400 MHz, DMSO-d6) δ 8.26 (s, 1H), 7.94 (s, 3H), 7.40-7.11 (m, 6H), 6.99-6.82 (m, 1H), 6.73 (s, 1H), 6.62 (s, 1H), 6.27-6.22 (m, 1H), 4.74-4.64 (m, 1H), 3.96 (d, J=16.1 Hz, 1H), 3.74-7.70 (m, 1H), 3.52 (s, 1H), 2.89 (m, 1H), 2.73 (m, 1H), 2.30 (s, 3H) ppm. MS: M/e 523 (M+1)⁺.

### Compound B20: 2-(5-amino-2-(furan-2-yl)-7H-pyrazolo[4,3-e][1,2,4]triazolo[1,5-c]pyrimidin-7-yl)-N,N-dimethyl-2-phenylpropanamide

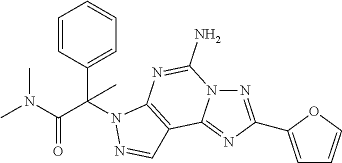 [SMILES: CN(C)C(=O)C(C)(c1ccccc1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)-2-phenylpropanoic acid (30 mg, 0.077 mmol), dimethylamine hydrochloride (6.3 mg, 0.077 mmol), HATU (35.3 mg, 0.092 mmol) and DIPEA (20 mg, 0.154 mmol) in DMF (3 mL) was stirred overnight. The reaction mixture was poured into H₂O (10 mL) and extracted with EtOAc (10 mL×3). The combined organic layers were washed with brine, dried over Na₂SO₄, concentrated and purified by prep-TLC (CH2Cl2/MeOH=10:1) to give the target compound (17 mg, 53%). ¹H NMR (400 MHz, DMSO-d6) δ 8.27 (d, J=1.7 Hz, 1H), 8.05 (s, 2H), 7.96 (s, 1H), 7.37-7.23 (m, 4H), 7.19-7.14 (m, 2H), 6.79-6.70 (m, 1H), 2.90 (s, 3H), 2.32 (s, 6H) ppm. MS: M/e 417 (M+1)⁺.

### Compound B21: 2-(5-amino-2-(furan-2-yl)-7H-pyrazolo[4,3-e][1,2,4]triazolo[1,5-c]pyrimidin-7-yl)-2-phenyl-1-(piperazin-1-yl)propan-1-one

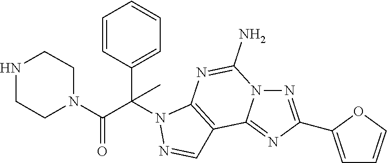 [SMILES: CC(C(=O)N1CCNCC1)(c1ccccc1)n1ncc2c1nc(N)n1nc(-c3ccco3)nc21]

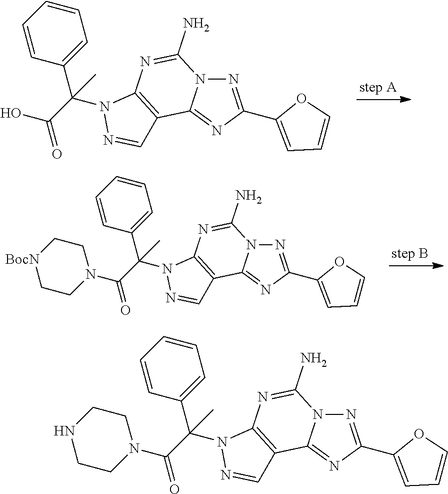

### Step A: tert-butyl 4-(2-(5-amino-2-(furan-2-yl)-7H-pyrazolo[4,3-e][1,2,4]triazolo[1,5-c]pyrimidin-7-yl)-2-phenylpropanoyl)piperazine-1-carboxylate

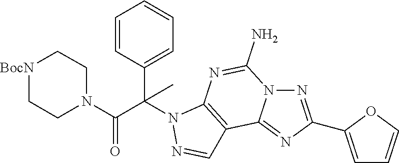 [SMILES: CC(C)(C)OC(=O)N1CCN(C(=O)C(C)(c2ccccc2)n2ncc3c2nc(N)n2nc(-c4ccco4)nc32)CC1]

A mixture of 2-(5-amino-2-(furan-2-yl)-7H-pyrazolo[4,3-e][1,2,4]triazolo[1,5-c]pyrimidin-7-yl)-2-phenylpropanoic acid (100 mg, 0.2570 mmol), tert-butyl piperazine-1-carboxylate (57 mg, 0.3085 mmol), HATU (146 mg, 0.3856 mmol), DIPEA (99 mg, 0.7712 mmol) in DMF (3 mL) was stirred at rt for 3 hours. The reaction mixture was poured into H₂O (10 mL) and extracted with EtOAc (15 mL×3). The combined organic layers were washed with brine, dried over Na₂SO₄, concentrated and purified by column chromatography (DCM/MeOH=40:1˜10:1) to give the product (96 mg, 67.0%) as white solid. MS: M/e 558 (M+1)⁺.

### Step B: 2-(5-amino-2-(furan-2-yl)-7H-pyrazolo[4,3-e][1,2,4]triazolo[1,5-c]pyrimidin-7-yl)-2-phenyl-1-(piperazin-1-yl)propan-1-one

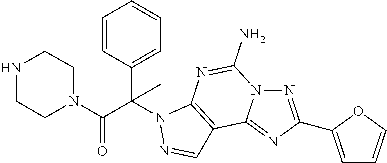 [SMILES: CC(C(=O)N1CCNCC1)(c1ccccc1)n1ncc2c1nc(N)n1nc(-c3ccco3)nc21]

A mixture of tert-butyl 4-(2-(5-amino-2-(furan-2-yl)-7H-pyrazolo[4,3-e][1,2,4]triazolo[1,5-c]pyrimidin-7-yl)-2-phenylpropanoyl)piperazine-1-carboxylate (96 mg, 0.1723 mmol) in solution of HCl in dioxane (3 mL, 4 mol/L) was stirred at rt for 3 hours. The reaction mixture was concentrated and purified by column chromatography (DCM/MeOH=40:1˜10:1) to give the product (63 mg, 79.98%) as white solid. ¹H NMR (400 MHz, DMSO-d6) δ 8.31 (s, 1H), 8.12 (s, 2H), 7.96 (s, 1H), 7.31 (dd, J=15.9, 8.3 Hz, 3H), 7.25 (d, J=3.2 Hz, 1H), 7.17 (d, J=7.1 Hz, 2H), 6.75 (d, J=1.4 Hz, 1H), 3.58 (s, 2H), 3.19-2.63 (m, 6H), 2.30 (s, 3H). MS: Me 458 (M+1)⁺.

### Compound B22: 2-(5-amino-2-(furan-2-yl)-7H-pyrazolo[4,3-e][1,2,4]triazolo[1,5-c]pyrimidin-7-yl)-2-phenyl-1-(2-oxa-6-azaspiro[3.3]heptan-6-yl)propan-1-one

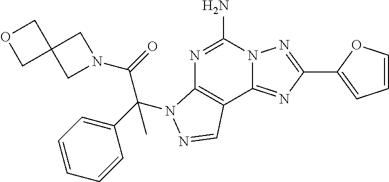 [SMILES: CC(C(=O)N1CC2(COC2)C1)(c1ccccc1)n1ncc2c1nc(N)n1nc(-c3ccco3)nc21]

A mixture of 2-oxa-6-azaspiro[3.3]heptane (15 mg, 0.15 mmol), 2-(5-amino-2-(furan-2-yl)-7H-pyrazolo[4,3-e][1,2,4]triazolo[1,5-c]pyrimidin-7-yl)-2-phenylpropanoic acid (40 mg, 0.1 mmol), HATU (43 mg, 0.11 mmol) and DIPEA (0.2 mL) in DMF (2 mL) was stirred at RT overnight. The reaction mixture was poured into water (10 mL) and extracted with EA (15 mL×3). The combined organic layers were washed with brine, dried over Na₂SO₄ and concentrated under reduced pressure. The residue was purified by prep-TLC (EA:100%) to afford the title compound as white solid (19 mg, yield: 39.3%). ¹H NMR (400 MHz, DMSO-d6) δ 8.30 (s, 1H), 8.04 (br.s, 2H), 7.97-7.89 (m, 1H), 7.35-7.21 (m, 4H), 7.07-6.90 (m, 2H), 6.81-6.66 (m, 1H), 4.69-4.42 (m, 3H), 4.35 (s, 1H), 4.24-4.03 (m, 2H), 3.83-3.68 (m, 1H), 3.10-2.93 (m, 1H), 2.30 (s, 3H) ppm. MS: M/e 471 (M+1)⁺.

### Compound B23: 2-(5-amino-2-(furan-2-yl)-7H-pyrazolo[4,3-e][1,2,4]triazolo[1,5-c]pyrimidin-7-yl)-2-phenyl-1-(pyrrolidin-1-yl)propan-1-one

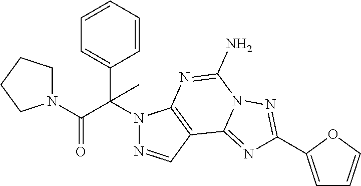 [SMILES: CC(C(=O)N1CCCC1)(c1ccccc1)n1ncc2c1nc(N)n1nc(-c3ccco3)nc21]

To a mixture of 2-(5-amino-2-(furan-2-yl)-7H-pyrazolo[4,3-e][1,2,4]triazolo[1,5-c]pyrimidin-7-yl)-2-phenylpropanoic acid (40 mg, 0.1 mmol), pyrrolidine (10 mg, 0.14 mmol), DIEA (55 mg, 0.42 mmol) in DMF (1 mL) was added HATU (46 mg, 0.12 mmol) at rt and the mixture was stirred at rt for 16 hrs. The mixture was diluted with 10 mL of EA, washed with brine (5 mL×3), dried over Na₂SO₄, concentrated and the resulting oil was purified by prep-TLC (EA: 100%) to give the title product (8.0 mg, yield: 18%) after having been lyophilized. ¹H NMR (400 MHz, DMSO-d6) δ 8.25 (d, J=1.6 Hz, 1H), 8.05 (s, 2H), 7.96 (s, 1H), 7.38-7.21 (m, 4H), 7.14 (d, J=8.0 Hz, 2H), 6.77-6.71 (m, 1H), 3.55-3.38 (m, 2H), 2.67-2.59 (m, 1H), 2.40-2.30 (m, 4H), 1.69-1.49 (m, 4H). MS: M/e 443 (M+1)⁺.

### Compound B24: 2-(5-amino-2-(furan-2-yl)-7H-pyrazolo[4,3-e][1,2,4]triazolo[1,5-c]pyrimidin-7-yl)-1-(azetidin-1-yl)-2-phenylpropan-1-one

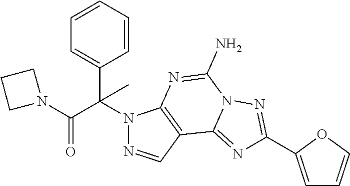 [SMILES: CC(C(=O)N1CCC1)(c1ccccc1)n1ncc2c1nc(N)n1nc(-c3ccco3)nc21]

To a mixture of 2-(5-amino-2-(furan-2-yl)-7H-pyrazolo[4,3-e][1,2,4]triazolo[1,5-c]pyrimidin-7-yl)-2-phenylpropanoic acid (40 mg, 0.1 mmol), azetidine (10 mg, 0.17 mmol), DIEA (55 mg, 0.42 mmol) in DMF (1 mL) was added HATU (46 mg, 0.12 mmol) at rt and the mixture was stirred at rt for 16 hrs. The mixture was diluted with 10 mL of EA, washed with brine (5 mL×3), dried over Na₂SO₄, concentrated and the resulting oil was purified by prep-TLC (EA: 100%) to give the title product (21 mg, yield: 49%) after having been lyophilized. ¹H NMR (400 MHz, DMSO-d6) δ 8.29 (s, 1H), 8.05 (s, 2H), 7.96 (s, 1H), 7.39-7.18 (m, 4H), 7.04 (d, J=7.2 Hz, 2H), 6.75 (s, 1H), 4.11-3.77 (m, 2H), 3.67-3.47 (m, 1H), 3.04-2.80 (m, 1H), 2.32 (s, 3H), 2.07-1.90 (m, 2H). MS: M/e 429 (M+1)⁺.

### Compound B25: 2-(5-amino-2-(furan-2-yl)-7H-pyrazolo[4,3-e][1,2,4]triazolo[1,5-c]pyrimidin-7-yl)-1-(4-hydroxy-4-methylpiperidin-1-yl)-2-phenylpropan-1-one

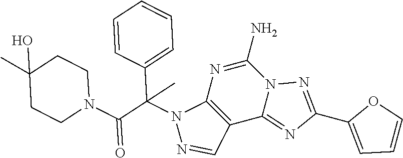 [SMILES: CC1(O)CCN(C(=O)C(C)(c2ccccc2)n2ncc3c2nc(N)n2nc(-c4ccco4)nc32)CC1]

To a stirred solution of 2-(5-amino-2-(furan-2-yl)-7H-pyrazolo[4,3-e][1,2,4]triazolo[1,5-c]pyrimidin-7-yl)-2-phenylpropanoic acid (100 mg, 0.26 mmol), HATU (108 mg, 0.28 mmol) and DIEA (100 mg, 0.78 mmol) in THF (15 ml) was added 4-methylpiperidin-4-ol (33 mg, 0.28 mmol). The reaction mixture was stirred at rt for 15 h. After completion, the reaction mixture was diluted with EA (30 ml) and then washed with H₂O (15 ml×2). The organic layer was dried over Na₂SO₄, filtered and then concentrated under reduced pressure to afford a residue. The residue was purified by prep-TLC with PE:EA (1:5) to afford the product (6.65 mg). ¹H NMR (400 MHz, DMSO-d6) δ 8.28 (s, 1H), 8.07 (s, 2H), 7.96 (s, 1H), 7.36-7.28 (m, 3H), 7.24 (d, J=4 Hz, 1H), 7.19 (d, J=8 Hz, 2H), 6.74 (dd, J=4 Hz, 2 Hz, 1H), 4.22 (s, 1H), 4.18-3.95 (m, 1H), 3.16-3.01 (m, 1H), 2.99-2.78 (m, 2H), 2.28 (s, 3H), 1.19-1.16 (m, 4H), 0.92 (s, 3H) ppm. MS: M/e 487 (M+1)⁺.

### Compound B26: 2-(5-amino-2-(furan-2-yl)-7H-pyrazolo[4,3-e][1,2,4]triazolo[1,5-c]pyrimidin-7-yl)-2-phenyl-1-(7-oxa-2-azaspiro[3.5]nonan-2-yl)propan-1-one

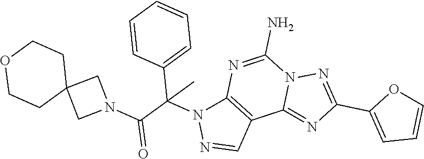 [SMILES: CC(C(=O)N1CC2(CCOCC2)C1)(c1ccccc1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)-2-phenylpropanoic acid (100 mg, 0.2570 mmol), 7-oxa-2-azaspiro[3.5]nonane (39 mg, 0.3085 mmol), HATU (146 mg, 0.3856 mmol), DIPEA (99 mg, 0.7712 mmol) in DMF (3 mL) was stirred at rt for 3 hours. The reaction mixture was poured into H₂O (10 mL) and extracted with EtOAc (15 mL×3). The combined organic layers were washed with brine, dried over Na₂SO₄, concentrated and purified by column chromatography (DCM/MeOH=40:1˜10:1) to give the product (62 mg, 48.43%) as white solid. ¹H NMR (400 MHz, DMSO-d6) δ 8.28 (s, 1H), 8.07 (s, 2H), 7.96 (s, 1H), 7.32-7.23 (m, 4H), 7.04 (d, J=6.5 Hz, 2H), 6.75 (dd, J=3.3, 1.7 Hz, 1H), 3.72 (q, J=9.6 Hz, 2H), 3.47-3.33 (m, 4H), 2.33 (s, 3H), 1.48 (dd, J=12.5, 6.0 Hz, 2H), 1.36-1.20 (m, 4H). MS: M/e 499 (M+1)⁺.

### Compound B27: 2-(5-amino-2-(furan-2-yl)-7H-pyrazolo[4,3-e][1,2,4]triazolo[1,5-c]pyrimidin-7-yl)-1-(4-methylpiperazin-1-yl)-2-phenylpropan-1-one

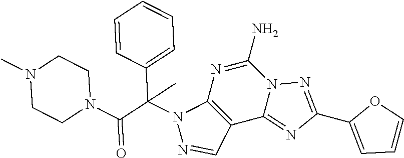 [SMILES: CN1CCN(C(=O)C(C)(c2ccccc2)n2ncc3c2nc(N)n2nc(-c4ccco4)nc32)CC1]

A mixture of 2-(5-amino-2-(furan-2-yl)-7H-pyrazolo[4,3-e][1,2,4]triazolo[1,5-c]pyrimidin-7-yl)-2-phenylpropanoic acid (100 mg, 0.2570 mmol), 1-methylpiperazine (31 mg, 0.3085 mmol), HATU (146 mg, 0.3856 mmol), DIPEA (99 mg, 0.7712 mmol) in DMF (3 mL) was stirred at rt for 3 hours. The reaction mixture was poured into H₂O (10 mL) and extracted with EtOAc (15 mL×3). The combined organic layers were washed with brine, dried over Na₂SO₄, concentrated and purified by column chromatography (DCM/MeOH=40:1˜10:1) to give the product (76 mg, 62.8%) as white solid. ¹H NMR (400 MHz, DMSO-d6) δ 8.32 (s, 1H), 8.10 (s, 2H), 7.96 (d, J=0.9 Hz, 1H), 7.35-7.27 (m, 3H), 7.25 (d, J=3.3 Hz, 1H), 7.13 (s, 2H), 6.75 (dd, J=3.4, 1.8 Hz, 1H), 3.59 (dd, J=10.1, 6.2 Hz, 2H), 3.33 (s, 3H), 3.11 (dd, J=7.2, 4.2 Hz, 2H), 2.33 (s, 3H). MS: M/e 472 (M+1)⁺.

### Compound B28: 2-(5-amino-2-(furan-2-yl)-7H-pyrazolo[4,3-e][1,2,4]triazolo[1,5-c]pyrimidin-7-yl)-1-((S)-3-methoxypiperidin-1-yl)-2-phenylpropan-1-one

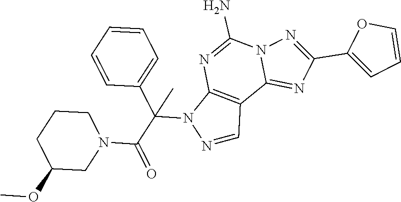 [SMILES: CO[C@H]1CCCN(C(=O)C(C)(c2ccccc2)n2ncc3c2nc(N)n2nc(-c4ccco4)nc32)C1]

A mixture of 2-(5-amino-2-(furan-2-yl)-7H-pyrazolo[4,3-e][1,2,4]triazolo[1,5-c]pyrimidin-7-yl)-2-phenylpropanoic acid (30 mg, 0.077 mmol), (S)-3-methoxypiperidine (8.9 mg, 0.077 mmol), HATU (35 mg, 0.092 mmol) and DIPEA (30 mg, 0.23 mmol) in DMF (5 mL) was stirred overnight at RT. The reaction mixture was poured into H₂O (60 mL) and extracted with EA (20 mL×3). The combined organic layers were washed with brine, dried over Na₂SO₄, concentrated in vacuo and purified by column chromatography (EA) to give the target compound (20 mg, 53.3%). ¹H NMR (400 MHz, DMSO-d6) δ 8.27 (s, 1H), 8.07 (s, 2H), 7.96 (s, 1H), 7.31-7.20 (m, 6H), 6.74 (dd, J=3.1, 1.7 Hz, 1H), 3.97 (s, 1H), 3.3-3.14 (m, 4H), 2.82-2.67 (m, 3H), 2.29 (d, J=15.4 Hz, 3H), 1.66 (m, 2H), 1.23 (m, 2H). MS: M/e 487 (M+1)⁺.

### Compound B29: 2-(5-amino-2-(furan-2-yl)-7H-pyrazolo[4,3-e][1,2,4]triazolo[1,5-c]pyrimidin-7-yl)-1-((S)-3-methoxypyrrolidin-1-yl)-2-phenylpropan-1-one

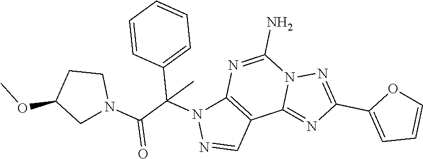 [SMILES: CO[C@H]1CCN(C(=O)C(C)(c2ccccc2)n2ncc3c2nc(N)n2nc(-c4ccco4)nc32)C1]

To a stirred solution of 2-(5-amino-2-(furan-2-yl)-7H-pyrazolo[4,3-e][1,2,4]triazolo[1,5-c]pyrimidin-7-yl)-2-phenylpropanoic acid (50 mg, 0.13 mmol), HATU (54 mg, 0.14 mmol) and DIEA (50 mg, 0.39 mmol) in THF (15 ml) was added (S)-3-methoxypyrrolidine hydrochloride (18 mg, 0.13 mmol). The reaction mixture was stirred at rt for 15 h. After completion, the reaction mixture was diluted with EA (30 ml) and then washed with H₂O (15 ml×2). The organic layer was dried over Na₂SO₄, filtered and then concentrated under reduced pressure to afford a residue. The residue was purified by prep-TLC with PE:EA (1:5) to afford the product (30.1 mg, 50%). ¹H NMR (400 MHz, DMSO-d6) δ 8.30-8.22 (m, 1H), 8.04 (s, 2H), 7.96 (s, 1H), 7.36-7.10 (m, 6H), 6.81-6.70 (m, 1H), 3.83-3.47 (m, 3H), 3.32-3.23 (m, 2H), 3.15-2.86 (m, 3H), 2.40-2.24 (m, 4H), 1.70-1.67 (m, 1H) ppm. MS: M/e 473 (M+1)⁺.

### Compound B30: 2-(5-amino-2-(furan-2-yl)-7H-pyrazolo[4,3-e][1,2,4]triazolo[1,5-c]pyrimidin-7-yl)-1-((R)-3-methoxypyrrolidin-1-yl)-2-phenylpropan-1-one

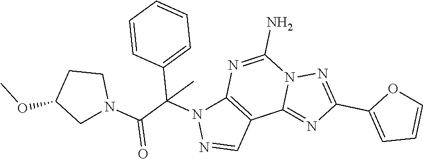 [SMILES: CO[C@@H]1CCN(C(=O)C(C)(c2ccccc2)n2ncc3c2nc(N)n2nc(-c4ccco4)nc32)C1]

To a stirred solution of 2-(5-amino-2-(furan-2-yl)-7H-pyrazolo[4,3-e][1,2,4]triazolo[1,5-c]pyrimidin-7-yl)-2-phenylpropanoic acid (50 mg, 0.13 mmol), HATU (54 mg, 0.14 mmol) and DIEA (50 mg, 0.39 mmol) in THF (15 ml) was added (R)-3-methoxypyrrolidine hydrochloride (18 mg, 0.13 mmol). The reaction mixture was stirred at rt for 15 h. After completion, the reaction mixture was diluted with EA (30 ml) and then washed with H₂O (15 ml×2). The organic layer was dried over Na₂SO₄, filtered and then concentrated under reduced pressure to afford a residue. The residue was purified by prep-TLC with PE:EA (1:5) to afford the product (26.5 mg, 44%). ¹H NMR (400 MHz, DMSO-d6) δ 8.30-8.22 (m, 1H), 8.05 (s, 2H), 7.95 (s, 1H), 7.37-7.11 (m, 6H), 6.80-6.70 (m, 1H), 3.83-3.44 (m, 3H), 3.32-3.30 (m, 2H), 3.15-2.86 (m, 3H), 2.40-2.28 (m, 4H), 1.71-1.66 (m, 1H) ppm. MS: M/e 473 (M+1)⁺.

### Compound B31: 2-(5-amino-2-(furan-2-yl)-7H-pyrazolo[4,3-e][1,2,4]triazolo[1,5-c]pyrimidin-7-yl)-1-((R)-3-methoxypiperidin-1-yl)-2-phenylpropan-1-one

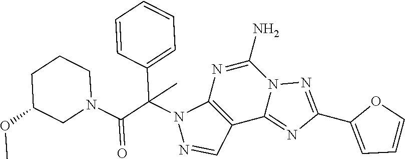 [SMILES: CO[C@@H]1CCCN(C(=O)C(C)(c2ccccc2)n2ncc3c2nc(N)n2nc(-c4ccco4)nc32)C1]

To a stirred solution of 2-(5-amino-2-(furan-2-yl)-7H-pyrazolo[4,3-e][1,2,4]triazolo[1,5-c]pyrimidin-7-yl)-2-phenylpropanoic acid (50 mg, 0.13 mmol), HATU (54 mg, 0.14 mmol) and DIEA (50 mg, 0.39 mmol) in THF (15 ml) was added (R)-3-methoxypiperidine (15 mg, 0.13 mmol). The reaction mixture was stirred at rt for 15 h. After completion, the reaction mixture was diluted with EA (30 ml) and then washed with H₂O (15 ml×2). The organic layer was dried over Na₂SO₄, filtered and then concentrated under reduced pressure to afford a residue. The residue was purified by prep-TLC with PE:EA (1:5) to afford the product (15.6 mg, 25%). ¹H NMR (400 MHz, DMSO-d6) δ 8.27 (s, 1H), 8.07 (s, 2H), 7.96 (s, 1H), 7.38-7.12 (m, 6H), 6.74 (dd, J=4 Hz, 2 Hz, 1H), 4.28-3.86 (m, 1H), 3.28 (s, 3H), 3.26-3.08 (m, 2H), 2.85-2.70 (m, 2H), 2.36-2.16 (m, 4H), 1.73-1.59 (m, 1H), 1.31-1.03 (m, 2H) ppm. MS: M/e 487 (M+1)⁺.

### Compound B32: 2-(5-amino-2-(furan-2-yl)-7H-pyrazolo[4,3-e][1,2,4]triazolo[1,5-c]pyrimidin-7-yl)-1-((R)-3-hydroxypiperidin-1-yl)-2-phenylpropan-1-one

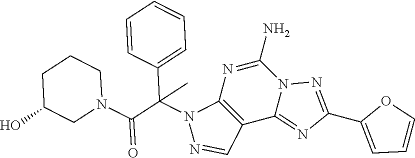 [SMILES: CC(C(=O)N1CCC[C@@H](O)C1)(c1ccccc1)n1ncc2c1nc(N)n1nc(-c3ccco3)nc21]

To a stirred solution of 2-(5-amino-2-(furan-2-yl)-7H-pyrazolo[4,3-e][1,2,4]triazolo[1,5-c]pyrimidin-7-yl)-2-phenylpropanoic acid (50 mg, 0.13 mmol), HATU (54 mg, 0.14 mmol) and DIEA (50 mg, 0.39 mmol) in THF (15 ml) was added (R)-piperidin-3-ol hydrochloride (18 mg, 0.13 mmol). The reaction mixture was stirred at rt for 15 h. After completion, the reaction mixture was diluted with EA (30 ml) and then washed with H₂O (15 ml×2). The organic layer was dried over Na₂SO₄, filtered and then concentrated under reduced pressure to afford a residue. The residue was purified by prep-TLC with PE:EA (1:5) to afford the product (40.5 mg, 67%). ¹H NMR (400 MHz, DMSO-d6) δ 8.28 (s, 1H), 8.05 (s, 2H), 7.96 (s, 1H), 7.35-7.12 (m, 6H), 6.74 (dd, J=4 Hz, 2 Hz, 1H), 4.97-4.00 (m, 2H), 3.32-2.80 (m, 4H), 2.40-2.20 (m, 4H), 1.69-1.59 (m, 1H), 1.28-1.01 (m, 2H) ppm. MS: M/e 473 (M+1)⁺.

### Compound B33: methyl 7-(2-(5-amino-2-(furan-2-yl)-7H-pyrazolo[4,3-e][1,2,4]triazolo[1,5-c]pyrimidin-7-yl)-2-phenylpropanoyl)-7-azaspiro[3.5]nonane-2-carboxylate

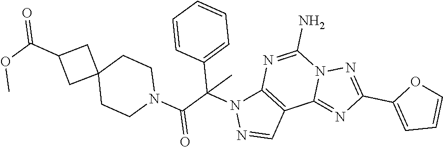 [SMILES: COC(=O)C1CC2(CCN(C(=O)C(C)(c3ccccc3)n3ncc4c3nc(N)n3nc(-c5ccco5)nc43)CC2)C1]

A mixture of 2-(5-amino-2-(furan-2-yl)-7H-pyrazolo[4,3-e][1,2,4]triazolo[1,5-c]pyrimidin-7-yl)-2-phenylpropanoic acid (100 mg, 0.2570 mmol), methyl 7-azaspiro[3.5]nonane-2-carboxylate (56 mg, 0.3085 mmol), HATU (146 mg, 0.3856 mmol), DIPEA (99 mg, 0.7712 mmol) in DMF (3 mL) was stirred at rt for 3 hours. The reaction mixture was poured into H₂O (10 mL) and extracted with EtOAc (15 mL×3). The combined organic layers were washed with brine, dried over Na₂SO₄, concentrated and purified by column chromatography (DCM/MeOH=40:1˜10:1) to give the product (74 mg, 57.8%) as white solid. ¹H NMR (400 MHz, DMSO-d6) δ 8.28 (s, 1H), 8.05 (s, 2H), 7.95 (d, J=0.9 Hz, 1H), 7.35-7.26 (m, 3H), 7.25 (d, J=3.4 Hz, 1H), 7.18 (d, J=7.1 Hz, 2H), 6.74 (dd, J=3.4, 1.8 Hz, 1H), 3.93 (s, 3H), 3.53 (s, 3H), 3.00 (s, 2H), 2.79 (s, 2H), 2.28 (s, 4H), 1.97-1.65 (m, J=46.8 Hz, 4H), 1.44 (d, J=40.8 Hz, 2H). MS: M/e 555 (M+1)⁺.

### Compound B34: 2-(5-amino-2-(furan-2-yl)-7H-pyrazolo[4,3-e][1,2,4]triazolo[1,5-c]pyrimidin-7-yl)-2-phenyl-1-(2-oxa-7-azaspiro[3.5]nonan-7-yl propan-1-one

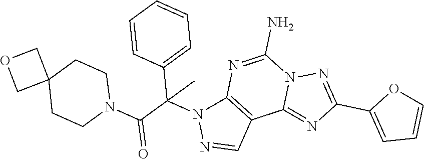 [SMILES: CC(C(=O)N1CCC2(CC1)COC2)(c1ccccc1)n1ncc2c1nc(N)n1nc(-c3ccco3)nc21]

A mixture of 2-(5-amino-2-(furan-2-yl)-7H-pyrazolo[4,3-e][1,2,4]triazolo[1,5-c]pyrimidin-7-yl)-2-phenylpropanoic acid (100 mg, 0.2570 mmol), 2-oxa-7-azaspiro[3.5]nonane (39 mg, 0.3085 mmol), HATU (146 mg, 0.3856 mmol), DIPEA (99 mg, 0.7712 mmol) in DMF (3 mL) was stirred at rt for 3 hours. The reaction mixture was poured into H₂O (10 mL) and extracted with EtOAc (15 mL×3). The combined organic layers were washed with brine, dried over Na₂SO₄, concentrated and purified by column chromatography (DCM/MeOH=40:1˜10:1) to give the product (74 mg, 57.8%) as white solid. ¹H NMR (400 MHz, DMSO-d6) δ 8.28 (s, 1H), 8.06 (s, 1H), 7.96 (s, 1H), 7.38-7.26 (m, 3H), 7.25 (d, J=3.3 Hz, 1H), 7.18 (d, J=7.0 Hz, 2H), 6.74 (dd, J=3.2, 1.7 Hz, 1H), 4.16 (s, 4H), 3.47 (s, 2H), 2.80 (s, 2H), 1.68 (s, 2H), 1.10 (d, J=36.9 Hz, 2H). MS: M/e 499 (M+1)⁺.

### Compound C1: 2-(5-amino-2-(furan-2-yl)-8H-pyrazolo[4,3-e][1,2,4]triazolo[1,5-c]pyrimidin-8-yl)-1-(4-(4-(2-methoxyethoxy)phenyl)piperazin-1-yl)propan-1-one

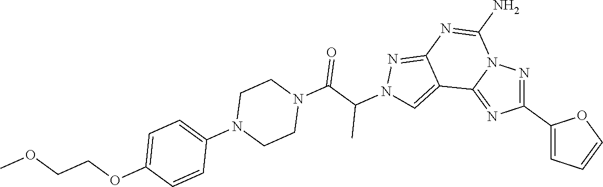 [SMILES: COCCOc1ccc(N2CCN(C(=O)C(C)n3cc4c(nc(N)n5nc(-c6ccco6)nc45)n3)CC2)cc1]

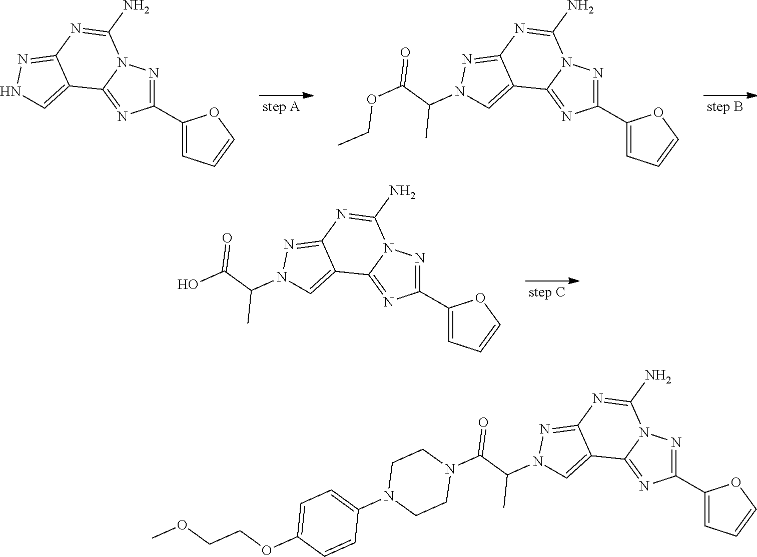 [SMILES: Nc1nc2n[nH]cc2c2nc(-c3ccco3)nn12 ; CCOC(=O)C(C)n1cc2c(nc(N)n3nc(-c4ccco4)nc23)n1 ; CC(C(=O)O)n1cc2c(nc(N)n3nc(-c4ccco4)nc23)n1 ; COCCOc1ccc(N2CCN(C(=O)C(C)n3cc4c(nc(N)n5nc(-c6ccco6)nc45)n3)CC2)cc1]

### Step A: ethyl 2-(5-amino-2-(furan-2-yl)-8H-pyrazolo[4,3-e][1,2,4]triazolo[1,5-c]pyrimidin-8-yl)propanoate

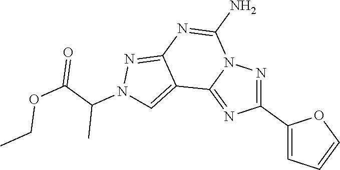 [SMILES: CCOC(=O)C(C)n1cc2c(nc(N)n3nc(-c4ccco4)nc23)n1]

To a stirred solution of 2-(furan-2-yl)-7H-pyrazolo[4,3-e][1,2,4]triazolo[1,5-c]pyrimidin-5-amine (500 mg, 2.07 mmol) in DMF (20 mL) and K₂CO₃ (344 mg, 2.49 mmol) was added ethyl 2-chloropropanoate (339 mg, 2.49 mmol) at RT. The mixture was stirred at rt overnight. The mixture was diluted with water (20 mL) and extracted with EA (20 mL×2). The residue (a mixture of N₁ and N₂ position compounds) was used into next step directly. MS: M/e 342 (M+1)⁺.

### Step B: 2-(5-amino-2-(furan-2-yl)-8H-pyrazolo[4,3-e][1,2,4]triazolo[1,5-c]pyrimidin-8-yl)propanoic Acid

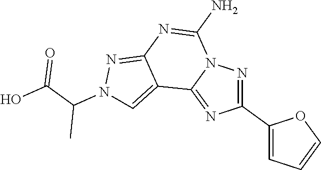 [SMILES: CC(C(=O)O)n1cc2c(nc(N)n3nc(-c4ccco4)nc23)n1]

To a stirred solution of the product of Step A (707 mg, crude, 2.07 mmol) in MeOH (12 mL) was added aq.NaOH (2.0 M, 4 mL) at RT. After the addition, the mixture was stirred at 60° C. for 3 hours. The reaction was concentrated under reduced pressure. The residue was dissolved into water (20 mL) and neutralized by HCl (2M) to pH=3˜4. The water phase was concentrated under reduced pressure. The residue (a mixture of N₁ and N₂ position compounds, containing NaCl) was used into next step directly. MS: M/e 314 (M+1)⁺.

### Step C: 2-(5-amino-2-(furan-2-yl)-8H-pyrazolo[4,3-e][1,2,4]triazolo[1,5-c]pyrimidin-8-yl)-1-(4-(4-(2-methoxyethoxy)phenyl)piperazin-1-yl)propan-1-one

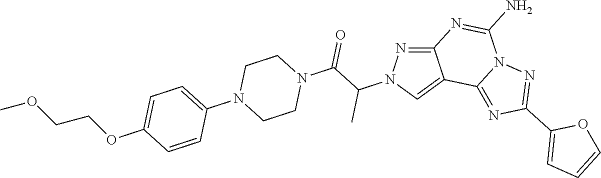 [SMILES: COCCOc1ccc(N2CCN(C(=O)C(C)n3cc4c(nc(N)n5nc(-c6ccco6)nc45)n3)CC2)cc1]

A mixture of the product of step B (200 mg, 0.64 mmol), 1-(4-(2-methoxyethoxy)phenyl)piperazine (190 mg, 0.7 mmol), HATU (267 mg, 0.7 mmol) and Et₃N (322 mg, 3.19 mmol) in DMF (10 mL) was stirred at RT overnight. The reaction mixture was poured into H₂O (20 mL) and extracted with EA (15 mL×3). The combined organic layers were washed with brine, dried over Na₂SO₄ and concentrated under reduced pressure. The residue was purified by prep-HPLC to afford the title compound as white solid. ¹H NMR (400 MHz, DMSO-d6) δ 8.76 (s, 1H), 7.98-7.89 (m, 1H), 7.71 (s, 2H), 7.23-7.15 (m, 1H), 7.04 (d, J=8.0 Hz, 2H), 6.89 (d, J=8.0 Hz, 2H), 6.76-6.70 (m, 1H), 5.86 (q, J=6.8 Hz, 1H), 4.07-4.00 (m, 2H), 3.91-3.73 (m, 2H), 3.72-3.66 (m, 2H), 3.64-3.59 (m, 2H), 3.30 (s, 3H), 3.25-3.00 (m, 4H), 1.71 (d, J=6.9 Hz, 3H) ppm. MS: M/e 532 (M+1)⁺.

Compound C1 was separated into two enantiomeric stereoisomers, Compound C1a (earlier peak), and Compound C1b (later peak) by chiral prep-HPLC. The chiral separation conditions are shown below.

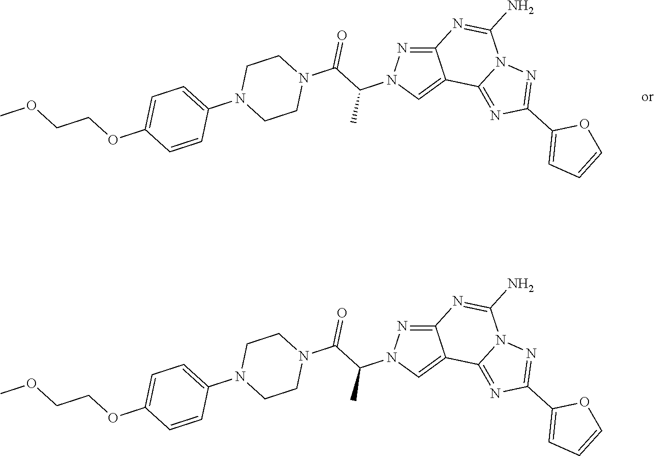 [SMILES: COCCOc1ccc(N2CCN(C(=O)[C@@H](C)n3cc4c(nc(N)n5nc(-c6ccco6)nc45)n3)CC2)cc1 ; COCCOc1ccc(N2CCN(C(=O)[C@H](C)n3cc4c(nc(N)n5nc(-c6ccco6)nc45)n3)CC2)cc1]

| Column | CHIRALPAK IF |
|---|---|
| Column size | 2*25 cm, 5 um |
| Injection | 3 ml |
| Mobile phase | (Hex:DCM = 1:1):IPA = 50:50 |
| Flow rate | 13 ml/min |
| Wavelength | UV 220 nm |
| Temperature | 35° C. |
| Sample solution | 6 mg/ml in MeOH:DCM = 2:1 |
| Prep-HPLC equipment | BJ-Prep-Gilson-HPLC |

### Compound C2: 2-(5-amino-2-(furan-2-yl)-8H-pyrazolo[4,3-e][1,2,4]triazolo[1,5-c]pyrimidin-8-yl)-1-(4-(4-(2-methoxyethoxy)phenyl)piperazin-1-yl)-3-methylbutan-1-one

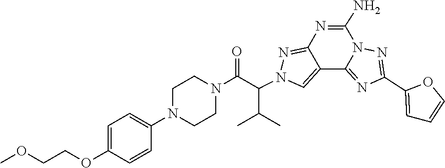 [SMILES: COCCOc1ccc(N2CCN(C(=O)C(C(C)C)n3cc4c(nc(N)n5nc(-c6ccco6)nc45)n3)CC2)cc1]

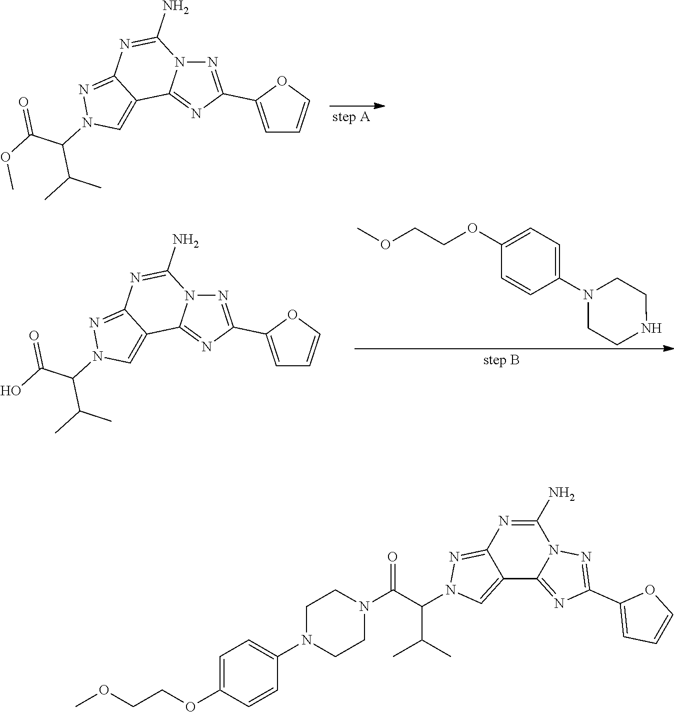 [SMILES: COC(=O)C(C(C)C)n1cc2c(nc(N)n3nc(-c4ccco4)nc23)n1 ; CC(C)C(C(=O)O)n1cc2c(nc(N)n3nc(-c4ccco4)nc23)n1 ; COCCOc1ccc(N2CCNCC2)cc1 ; COCCOc1ccc(N2CCN(C(=O)C(C(C)C)n3cc4c(nc(N)n5nc(-c6ccco6)nc45)n3)CC2)cc1]

### Step A: 2-(5-amino-2-(furan-2-yl)-8H-pyrazolo[4,3-e][1,2,4]triazolo[1,5-c]pyrimidin-8-yl)-3-methylbutanoic Acid

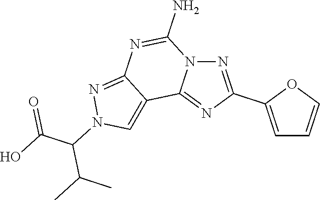 [SMILES: CC(C)C(C(=O)O)n1cc2c(nc(N)n3nc(-c4ccco4)nc23)n1]

To a stirred solution of methyl 2-(5-amino-2-(furan-2-yl)-8H-pyrazolo[4,3-e][1,2,4]triazolo[1,5-c]pyrimidin-8-yl)-3-methylbutanoate (150 mg, 0.42 mmol) in MeOH (4 mL) was added aqueous solution of NaOH (2 M, 2 mL) at rt and the resulting mixture was stirred for 2 hrs. The mixture was neutralized by HCl (1 M) and concentrated to dryness. 5 mL of a mixed solvent (CH₂Cl₂/MeOH=3:1) was added and stirred for 10 min. The suspension was filtered and the filtrate was concentrated to give the title product (125 mg, 87%) as a white solid. MS: M/e 342 (M+1)⁺.

### Step B: 2-(5-amino-2-(furan-2-yl)-8H-pyrazolo[4,3-e][1,2,4]triazolo[1,5-c]pyrimidin-8-yl)-1-(4-(4-(2-methoxyethoxy)phenyl)piperazin-1-yl)-3-methylbutan-1-one

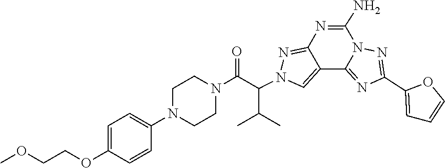 [SMILES: COCCOc1ccc(N2CCN(C(=O)C(C(C)C)n3cc4c(nc(N)n5nc(-c6ccco6)nc45)n3)CC2)cc1]

To a mixture of the product from step A (120 mg, 0.35 mmol), 1-(4-(2-methoxyethoxy)phenyl)piperazine (120 mg, 0.51 mmol) and DIEA (250 mg, 1.94 mmol) in DMF (3 mL) was added HATU (160 mg, 0.42 mmol) at rt and the mixture was stirred at rt for 2 hrs. 30 mL of EA was added and the mixture was washed with brine (10 mL×3), dried over Na₂SO₄ and concentrated. The resulting residue was purified by prep-TLC (EA/MeOH=20:1) to give the title product (57.0 mg, yield: 29%). ¹H NMR (400 MHz, DMSO-d6) δ 8.71 (d, J=1.2 Hz, 1H), 7.96 (s, 1H), 7.67 (s, 2H), 7.21 (d, J=3.6 Hz, 1H), 6.89 (d, J=8.0 Hz, 2H), 6.83 (d, J=8.8 Hz, 2H), 6.78-6.73 (m, 1H), 5.42 (d, J=10.0 Hz, 1H), 4.04-3.98 (m, 2H), 3.98-3.88 (m, 1H), 3.82-3.67 (m, 2H), 3.66-3.59 (m, 3H), 3.31 (s, 3H), 3.06-2.92 (m, 3H), 2.88-2.80 (m, 1H), 2.76-2.64 (m, 1H), 1.03 (d, J=6.4 Hz, 3H), 0.77 (d, J=6.8 Hz, 3H). MS: M/e 560 (M+1)⁺.

### Compound C3: 2-(5-amino-2-(furan-2-yl)-8H-pyrazolo[4,3-e][1,2,4]triazolo[1,5-c]pyrimidin-8-yl)-1-(4-(4-(2-methoxyethoxy)phenyl)piperazin-1-yl)-2-methylpropan-1-one

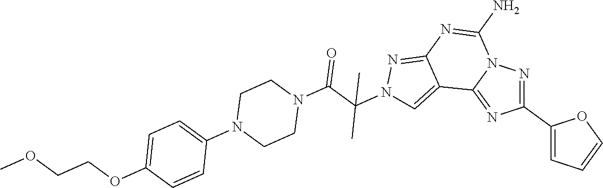 [SMILES: COCCOc1ccc(N2CCN(C(=O)C(C)(C)n3cc4c(nc(N)n5nc(-c6ccco6)nc45)n3)CC2)cc1]

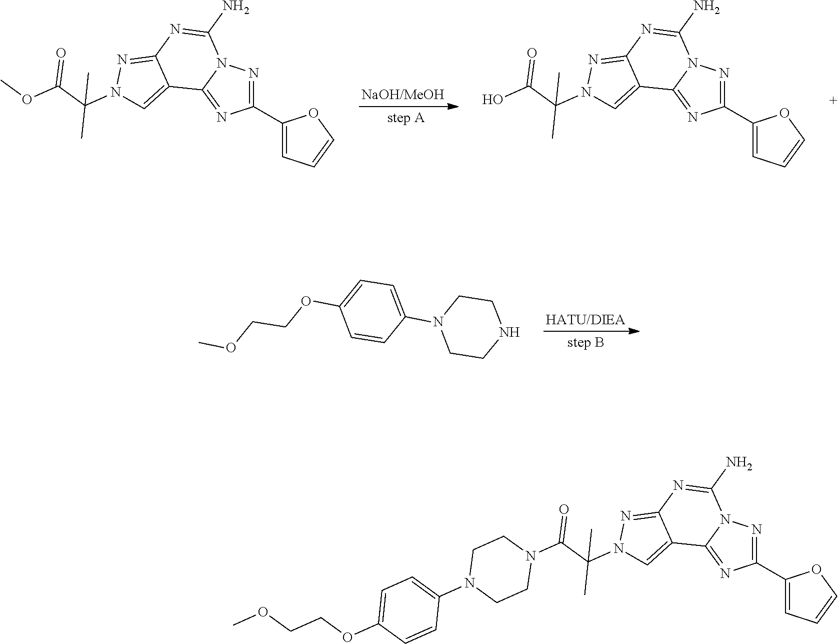 [SMILES: COC(=O)C(C)(C)n1cc2c(nc(N)n3nc(-c4ccco4)nc23)n1 ; CC(C)(C(=O)O)n1cc2c(nc(N)n3nc(-c4ccco4)nc23)n1 ; COCCOc1ccc(N2CCNCC2)cc1 ; COCCOc1ccc(N2CCN(C(=O)C(C)(C)n3cc4c(nc(N)n5nc(-c6ccco6)nc45)n3)CC2)cc1]

### Step A: 2-(5-amino-2-(furan-2-yl)-8H-pyrazolo[4,3-e][1,2,4]triazolo[1,5-c]pyrimidin-8-yl)-2-methylpropanoic Acid

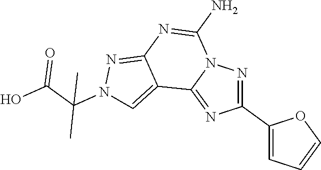 [SMILES: CC(C)(C(=O)O)n1cc2c(nc(N)n3nc(-c4ccco4)nc23)n1]

NaOH solution (352 mg, in 5 mL of water) was added to a solution of methyl 2-(5-amino-2-(furan-2-yl)-8H-pyrazolo[4,3-e][1,2,4]triazolo[1,5-c]pyrimidin-8-yl)-2-methylpropanoate (750 mg, 2.2 mmol) in methanol (10 mL). The reaction mixture was stirred at rt overnight. The solution was concentrated, added with water (10 mL) and acidified with 1N HCl solution to pH=5. The precipitated solid was filtered and dried to get the desired product as a white solid (670 mg, 93%). MS: M/e 328 (M+1)⁺

### Step B: 2-(5-amino-2-(furan-2-yl)-8H-pyrazolo[4,3-e][1,2,4]triazolo[1,5-c]pyrimidin-8-yl)-1-(4-(4-(2-methoxyethoxy)phenyl)piperazin-1-yl)-2-methylpropan-1-one

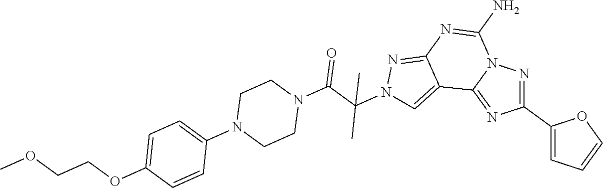 [SMILES: COCCOc1ccc(N2CCN(C(=O)C(C)(C)n3cc4c(nc(N)n5nc(-c6ccco6)nc45)n3)CC2)cc1]

A mixture of 2-(5-amino-2-(furan-2-yl)-8H-pyrazolo[4,3-e][1,2,4]triazolo[1,5-c]pyrimidin-8-yl)-2-methylpropanoic acid (100 mg, 0.3 mmol), 1-(4-(2-methoxyethoxy)phenyl)piperazine (72 mg, 0.3 mmol), HATU (137 mg, 0.36 mmol) and DIEA (78 mg, 0.6 mmol) in DMF (15 mL) was stirred at rt for 2 hrs. The solution was added with water (10 mL), extracted with ethyl acetate (20 mL) and washed with brine (20 mL). The organic layer was dried, concentrated and purified by column chromatography (PE:EA=1:1 to EA) to get the desired product as a white solid (135 mg, 81%). ¹H NMR (400 MHz, DMSO-d6, 80° C.) δ 8.74 (s, 1H), 7.87 (s, 1H), 7.41 (br.s, 2H), 7.15 (d, J=4.0 Hz, 1H), 6.76-6.74 (m, 4H), 6.69 (s, 1H), 3.97 (t, J=4.0 Hz, 2H), 3.58 (t, J=4.0 Hz, 2H), 3.10-3.29 (m, 4H), 3.28 (s, 3H), 2.83 (br.s, 4H), 1.83 (s, 6H) ppm. MS: M/e 546 (M+1)⁺

### Compound C4: 2-(5-amino-2-(furan-2-yl)-8H-pyrazolo[4,3-e][1,2,4]triazolo[1,5-c]pyrimidin-8-yl)-1-(4-(4-(2-methoxyethoxy)phenyl)piperazin-1-yl)butan-1-one

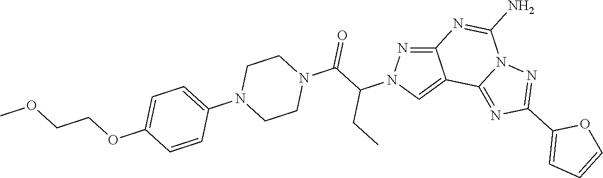 [SMILES: CCC(C(=O)N1CCN(c2ccc(OCCOC)cc2)CC1)n1cc2c(nc(N)n3nc(-c4ccco4)nc23)n1]

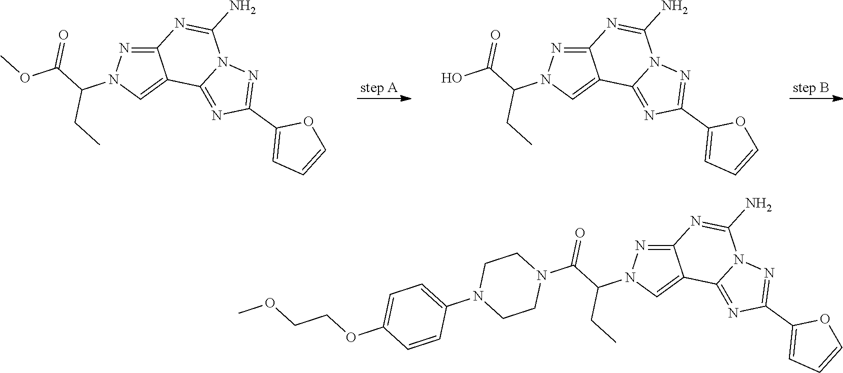

### Step A: 2-(5-amino-2-(furan-2-yl)-8H-pyrazolo[4,3-e][1,2,4]triazolo[1,5-c]pyrimidin-8-yl)butanoic Acid

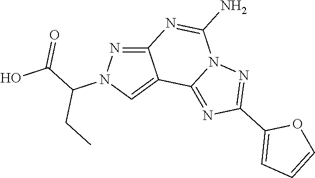 [SMILES: CCC(C(=O)O)n1cc2c(nc(N)n3nc(-c4ccco4)nc23)n1]

To a stirred mixture of methyl 2-(5-amino-2-(furan-2-yl)-8H-pyrazolo[4,3-e][1,2,4]triazolo[1,5-c]pyrimidin-8-yl)butanoate (100 mg, 0.29 mmol) in MeOH/H₂O (3.0 mL/0.5 mL) was added aq.NaOH (2.0 M, 0.5 mL). After the addition, the reaction mixture was stirred overnight. Most of solvent was removed to give the aqueous layer, then acidified to pH=3˜4 with aq.HCl and filtered, the cake was collected, dried to give the target compound (80 mg, 84.1%) as a white solid. MS: M/e 328 (M+1)⁺.

### Step B: 2-(5-amino-2-(furan-2-yl)-8H-pyrazolo[4,3-e][1,2,4]triazolo[1,5-c]pyrimidin-8-yl)-1-(4-(4-(2-methoxyethoxy)phenyl)piperazin-1-yl)butan-1-one

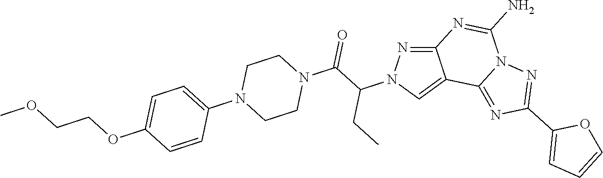 [SMILES: CCC(C(=O)N1CCN(c2ccc(OCCOC)cc2)CC1)n1cc2c(nc(N)n3nc(-c4ccco4)nc23)n1]

A mixture of the product of step A (100 mg, 0.31 mmol), 1-(4-(2-methoxyethoxy)phenyl)piperazine (80 mg, 0.24 mmol), HATU (120 mg, 0.32 mmol) and TEA (100 mg, 0.99 mmol) in CH₃CN (10 mL) was stirred for 16 hours at RT. The reaction mixture was poured into H₂O (20 mL) and extracted with EtOAc (20 mL×3). The combined organic layers were washed with brine, dried over Na₂SO₄, concentrated and purified by column chromatography (petroleum ether/EtOAc=1:1˜100% EtOAc) to give the target compound (21 mg, 15.5%) as a white solid. ¹H NMR (400 MHz, DMSO-d6) δ 8.74 (s, 1H), 7.95 (s, 1H), 7.65 (br, 2H), 7.19 (d, J=3.1 Hz, 1H), 6.92-6.78 (m, 4H), 6.74 (d, J=1.3 Hz, 1H), 5.73-5.60 (m, 1H), 4.02-3.96 (m, 2H), 3.76 (d, J=12.8 Hz, 2H), 3.66-3.58 (m, 4H), 3.34 (s, 3H), 3.10-2.83 (m, 4H), 2.22-2.09 (m, 2H), 0.87 (t, J=7.1 Hz, 3H). MS: M/e 546 (M+1)⁺.

### Compound C5: 2-(5-amino-2-(furan-2-yl)-8H-pyrazolo[4,3-e][1,2,4]triazolo[1,5-c]pyrimidin-8-yl)-1-(4-(4-(2-methoxyethoxy)phenyl)piperazin-1-yl)pentan-1-one

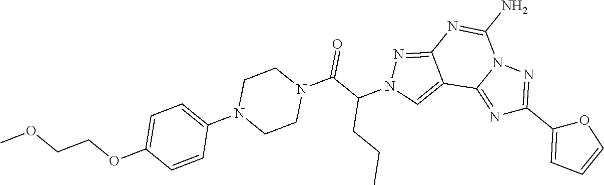 [SMILES: CCCC(C(=O)N1CCN(c2ccc(OCCOC)cc2)CC1)n1cc2c(nc(N)n3nc(-c4ccco4)nc23)n1]

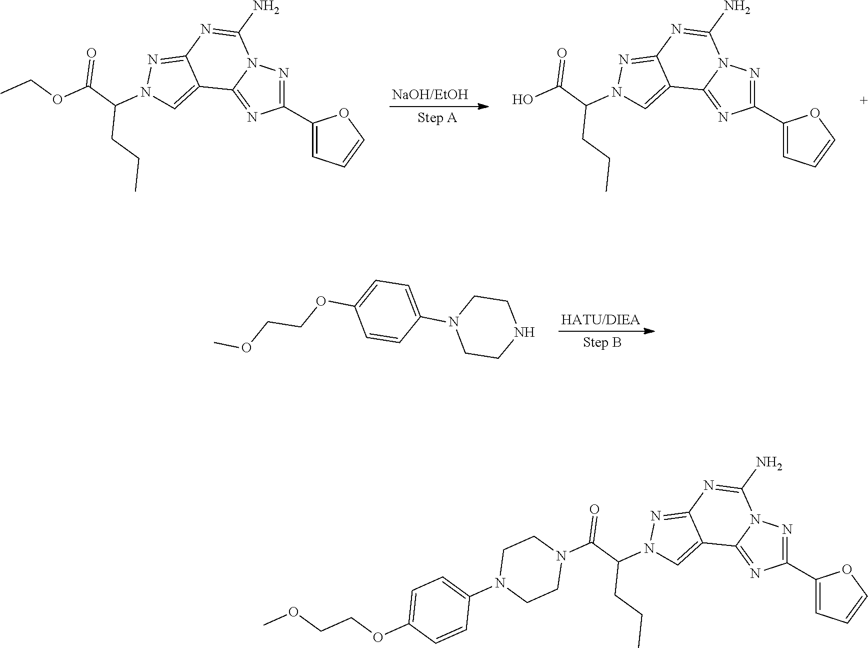 [SMILES: CCCC(C(=O)OCC)n1cc2c(nc(N)n3nc(-c4ccco4)nc23)n1 ; CCCC(C(=O)O)n1cc2c(nc(N)n3nc(-c4ccco4)nc23)n1 ; COCCOc1ccc(N2CCNCC2)cc1 ; CCCC(C(=O)N1CCN(c2ccc(OCCOC)cc2)CC1)n1cc2c(nc(N)n3nc(-c4ccco4)nc23)n1]

### Step A: 2-(5-amino-2-(furan-2-yl)-8H-pyrazolo[4,3-e][1,2,4]triazolo[1,5-c]pyrimidin-8-yl)pentanoic Acid

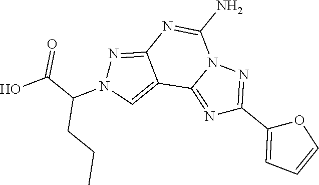 [SMILES: CCCC(C(=O)O)n1cc2c(nc(N)n3nc(-c4ccco4)nc23)n1]

NaOH solution (304 mg, in 2 mL of water) was added to a solution of ethyl 2-(5-amino-2-(furan-2-yl)-8H-pyrazolo[4,3-e][1,2,4]triazolo[1,5-c]pyrimidin-8-yl)pentanoate (700 mg, 1.9 mmol) in ethanol (10 mL). The reaction mixture was stirred at rt for 3 hrs. The solution was concentrated, added with water (10 mL) and acidified with 1N HCl solution to pH=5. The precipitated solid was filtered and dried to get the desired product as a white solid (600 mg, 92%) ppm. MS: M/e 342 (M+1)⁺

### Step B: 2-(5-amino-2-(furan-2-yl)-8H-pyrazolo[4,3-e][1,2,4]triazolo[1,5-c]pyrimidin-8-yl)-1-(4-(4-(2-methoxyethoxy)phenyl)piperazin-1-yl)pentan-1-one

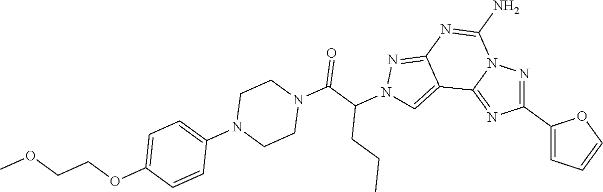 [SMILES: CCCC(C(=O)N1CCN(c2ccc(OCCOC)cc2)CC1)n1cc2c(nc(N)n3nc(-c4ccco4)nc23)n1]

A mixture of 2-(5-amino-2-(furan-2-yl)-8H-pyrazolo[4,3-e][1,2,4]triazolo[1,5-c]pyrimidin-8-yl)pentanoic acid (250 mg, 0.73 mmol), 1-(4-(2-methoxyethoxy)phenyl)piperazine (173 mg, 0.73 mmol), HATU (333 mg, 0.9 mmol) and DIEA (181 mg, 1.4 mmol) in DMF (20 mL) was stirred at rt for 2 hrs. The solution was added with water (10 mL), extracted with ethyl acetate (20 mL) and washed with brine (20 mL). The organic layer was dried, concentrated and purified by column chromatography (PE:EA=2:1 to EA) to get the desired product as a white solid (310 mg, 76%). ¹H NMR (400 MHz, DMSO-d6) δ 8.74 (s, 1H), 7.94 (s, 1H), 7.65 (br.s, 2H), 7.18 (d, J=4.0 Hz, 1H), 6.83-6.73 (m, 5H), 5.70 (dd, J=8.0 Hz, 4.0 Hz, 1H), 4.01-3.98 (m, 2H), 3.76-3.60 (m, 6H), 3.29 (s, 3H), 2.98-2.90 (m, 4H), 2.16-1.99 (m, 2H), 1.30-1.17 (m, 2H), 0.91 (t, J=8.0 Hz, 3H) ppm. MS: M/e 560 (M+1)⁺

Compound C5 was separated into two enantiomeric stereoisomers, Compound C5a (earlier peak), and Compound C5b (later peak) by chiral prep-HPLC. The chiral separation conditions are shown below.

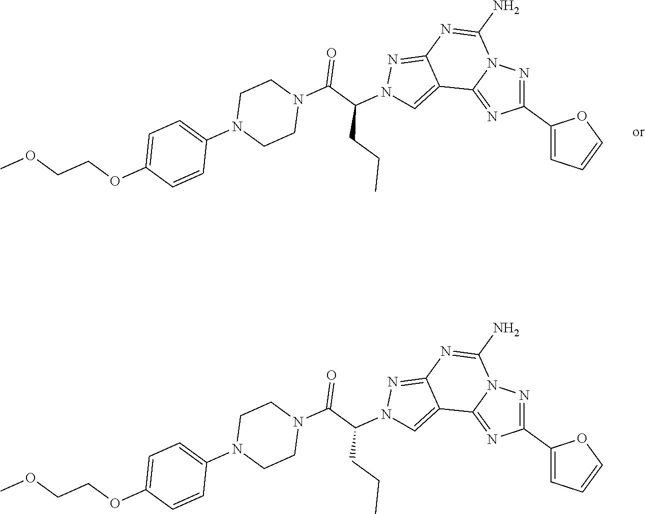 [SMILES: CCC[C@@H](C(=O)N1CCN(c2ccc(OCCOC)cc2)CC1)n1cc2c(nc(N)n3nc(-c4ccco4)nc23)n1 ; CCC[C@H](C(=O)N1CCN(c2ccc(OCCOC)cc2)CC1)n1cc2c(nc(N)n3nc(-c4ccco4)nc23)n1]

| Column | Chiralpak IA |
|---|---|
| Column size | 2 cm × 25 cm, 5 um |
| Injection | 0.7 mL |
| Mobile phase | (Hex:DCM = 3:1):EtOH = 50:50 |
| Flow rate | 14 ml/min |
| Wavelength | UV 220 nm |
| Temperature | 25° C. |
| Sample solution | 18.97 mg/ml in DCM:EtOH = 1:1 |
| Prep-HPLC equipment | Prep-Gilson-HPLC |

### Compound C6: 2-(5-amino-2-(furan-2-yl)-8H-pyrazolo[4,3-e][1,2,4]triazolo[1,5-c]pyrimidin-8-yl)-1-(4-(4-(2-(2-methoxyethoxy)ethoxy)phenyl)piperazin-1-yl)-2-methylpropan-1-one

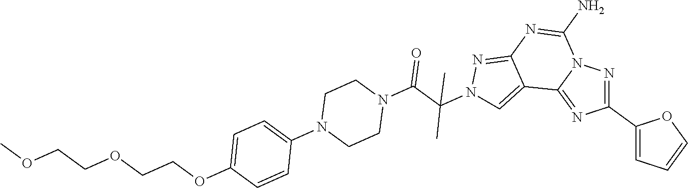 [SMILES: COCCOCCOc1ccc(N2CCN(C(=O)C(C)(C)n3cc4c(nc(N)n5nc(-c6ccco6)nc45)n3)CC2)cc1]

A mixture of 2-(5-amino-2-(furan-2-yl)-8H-pyrazolo[4,3-e][1,2,4]triazolo[1,5-c]pyrimidin-8-yl)-2-methylpropanoic acid (50 mg, 0.15 mmol), 1-(4-(2-(2-methoxyethoxy)ethoxy)phenyl)piperazine (43 mg, 0.15 mmol), HATU (69 mg, 0.18 mmol) and DIEA (39 mg, 0.3 mmol) in DMF (5 mL) was stirred at rt for 2 hrs. The solution was added with water (5 mL), extracted with ethyl acetate (10 mL) and washed with brine (10 mL). The organic layer was dried, concentrated and purified by column chromatography (PE:EA=1:1 to EA) to get the desired product (32 mg, 36%). ¹H NMR (400 MHz, DMSO-d6) δ 8.85 (s, 1H), 7.94 (s, 1H), 7.68 (br.s, 2H), 7.16 (d, J=4.0 Hz, 1H), 6.76 (s, 1H), 6.74-6.72 (m, 4H), 3.95 (t, J=4.0 Hz, 2H), 3.65 (t, J=4.0 Hz, 2H), 3.54-3.52 (m, 2H), 3.43-3.41 (m, 2H), 3.36 (br.s, 3H), 3.22 (s, 3H), 2.80 (br.s, 5H), 1.80 (s, 6H) ppm. MS: M/e 590 (M+1)⁺

### Compound C7: 2-(5-amino-2-(furan-2-yl)-8H-pyrazolo[4,3-e][1,2,4]triazolo[1,5-c]pyrimidin-8-yl)-1-(4-(4-(2-(2-methoxyethoxy)ethoxy)phenyl)piperazin-1-yl)pentan-1-one

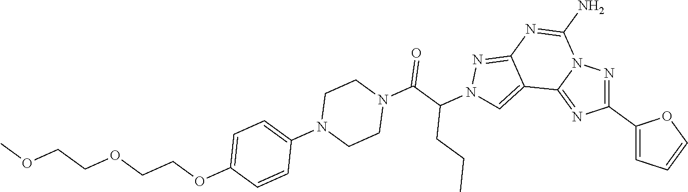 [SMILES: CCCC(C(=O)N1CCN(c2ccc(OCCOCCOC)cc2)CC1)n1cc2c(nc(N)n3nc(-c4ccco4)nc23)n1]

A mixture of 2-(5-amino-2-(furan-2-yl)-8H-pyrazolo[4,3-e][1,2,4]triazolo[1,5-c]pyrimidin-8-yl)pentanoic acid (50 mg, 0.15 mmol), 1-(4-(2-(2-methoxyethoxy)ethoxy)phenyl)piperazine (41 mg, 0.15 mmol), HATU (69 mg, 0.18 mmol) and DIEA (390 mg, 0.3 mmol) in DMF (5 mL) was stirred at rt for 2 hrs. The solution was added with water (5 mL), extracted with ethyl acetate (10 mL) and washed with brine (10 mL). The organic layer was dried, concentrated and purified by column chromatography (PE:EA=1:1 to EA) to get the desired product (38 mg, 42%). ¹H NMR (400 MHz, DMSO-d6) δ 8.74 (s, 1H), 7.94 (s, 1H), 7.65 (br.s, 2H), 7.18 (d, J=4.0 Hz, 1H), 6.84-6.73 (m, 5H), 5.70 (dd, J=8.0 Hz, 4.0 Hz, 1H), 3.99 (m, 2H), 3.70-3.41 (m, 10H), 3.24 (s, 3H), 2.99-2.91 (m, 4H), 2.17-1.99 (m, 2H), 1.30-1.18 (m, 2H), 0.91 (t, J=8.0 Hz, 3H) ppm. MS: M/e 604 (M+1)⁺

### Compound C8: 2-(5-amino-2-(furan-2-yl)-8H-pyrazolo[4,3-e][1,2,4]triazolo[1,5-c]pyrimidin-8-yl)-1-(4-(4-(2-(2-methoxyethoxy)ethoxy)phenyl)piperazin-1-yl)butan-1-one

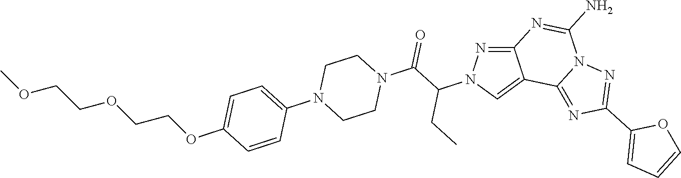 [SMILES: CCC(C(=O)N1CCN(c2ccc(OCCOCCOC)cc2)CC1)n1cc2c(nc(N)n3nc(-c4ccco4)nc23)n1]

A mixture of 2-(5-amino-2-(furan-2-yl)-8H-pyrazolo[4,3-e][1,2,4]triazolo[1,5-c]pyrimidin-8-yl)butanoic acid (50 mg, 0.15 mmol), 1-(4-(2-(2-methoxyethoxy)ethoxy)phenyl)piperazine (65 mg, 0.23 mmol), HATU (86 mg, 0.23 mmol) and TEA (30 mg, 0.30 mmol) in DMF (5 mL) was stirred overnight. The reaction mixture was poured into H₂O (20 mL) and extracted with EtOAc (20 mL×3). The combined organic layers were washed with brine, dried over Na₂SO₄, concentrated and purified by column chromatography (petroleum ether/EtOAc=1:1˜100% EtOAc) to give the target compound (21 mg, 23.7%). ¹H NMR (400 MHz, DMSO-d6) δ 8.74 (s, 1H), 7.94 (d, J=1.0 Hz, 1H), 7.67 (br., 2H), 7.19 (d, J=3.4 Hz, 1H), 6.94-6.83 (m, 4H), 6.73 (dd, J=3.4, 1.8 Hz, 1H), 5.64 (dd, J=8.8, 6.1 Hz, 1H), 4.03-3.98 (m, 2H), 3.80-3.76 (m, 2H), 3.68-3.65 (m, 4H), 3.56 (dd, J=5.7, 3.8 Hz, 2H), 3.45 (dd, J=5.7, 3.7 Hz, 2H), 3.24 (s, 3H), 3.15-2.89 (m, 4H), 2.25-2.06 (m, 2H), 0.87 (t, J=7.3 Hz, 3H) ppm. MS: M/e 590 (M+1)⁺.

### Compound C9: 2-(5-amino-2-(furan-2-yl)-8H-pyrazolo[4,3-e][1,2,4]triazolo[1,5-c]pyrimidin-8-yl)-1-(4-(4-(2-(2-methoxyethoxy)ethoxy)phenyl)piperazin-1-yl)propan-1-one

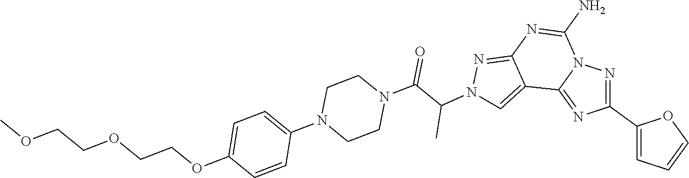 [SMILES: COCCOCCOc1ccc(N2CCN(C(=O)C(C)n3cc4c(nc(N)n5nc(-c6ccco6)nc45)n3)CC2)cc1]

A mixture of 1-(4-(2-(2-methoxyethoxy)ethoxy)phenyl)piperazine (60 mg, 0.21 mmol), 2-(5-amino-2-(furan-2-yl)-8H-pyrazolo[4,3-e][1,2,4]triazolo[1,5-c]pyrimidin-8-yl)propanoic acid (30 mg, 0.1 mmol), HATU (40 mg, 0.11 mmol) and DIPEA (0.4 mL, excess) in DMF (10 mL) was stirred at RT overnight. The reaction mixture was poured into water (20 mL) and extracted with EA (15 mL×3). The combined organic layers were washed with brine, dried over Na₂SO₄ and concentrated under reduced pressure. The residue was purified by prep-TLC (EA: 100%) to afford the title compound as white solid (5 mg, yield: 8.7%). ¹H NMR (400 MHz, DMSO-d6) δ 8.74 (s, 1H), 7.94 (s, 1H), 7.65 (br.s, 2H), 7.19 (s, 1H), 6.96-6.78 (m, 4H), 6.73 (s, 1H), 5.94-5.72 (m, 1H), 4.09-3.92 (m, 2H), 3.84-3.51 (m, 8H), 3.49-3.42 (m, 2H), 3.24 (s, 3H), 3.10-2.87 (m, 4H), 1.80-1.59 (m, 3H) ppm. MS: M/e 576 (M+1)⁺.

### Compound C10: 2-(5-amino-2-(furan-2-yl)-8H-pyrazolo[4,3-e][1,2,4]triazolo[1,5-c]pyrimidin-8-yl)-1-(4-(4-(2-methoxyethoxy)phenyl)piperazin-1-yl)hexan-1-one

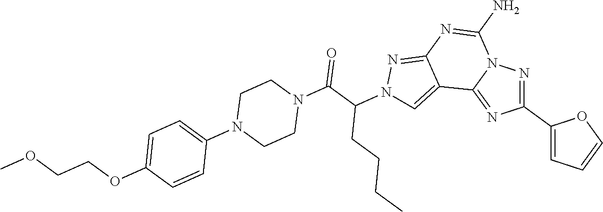 [SMILES: CCCCC(C(=O)N1CCN(c2ccc(OCCOC)cc2)CC1)n1cc2c(nc(N)n3nc(-c4ccco4)nc23)n1]

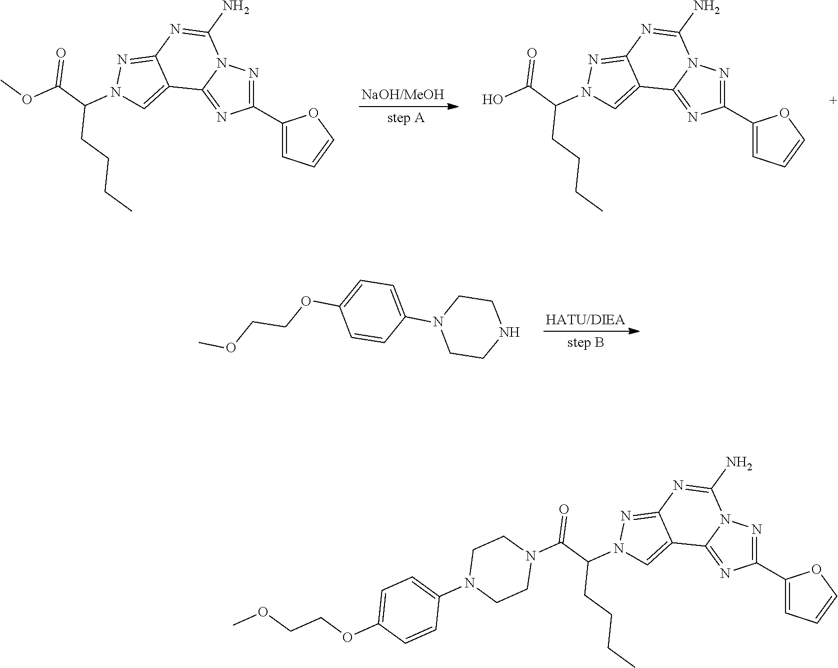 [SMILES: CCCCC(C(=O)OC)n1cc2c(nc(N)n3nc(-c4ccco4)nc23)n1 ; CCCCC(C(=O)O)n1cc2c(nc(N)n3nc(-c4ccco4)nc23)n1 ; COCCOc1ccc(N2CCNCC2)cc1 ; CCCCC(C(=O)N1CCN(c2ccc(OCCOC)cc2)CC1)n1cc2c(nc(N)n3nc(-c4ccco4)nc23)n1]

### Step A: 2-(5-amino-2-(furan-2-yl)-8H-pyrazolo[4,3-e][1,2,4]triazolo[1,5-c]pyrimidin-8-yl)hexanoic Acid

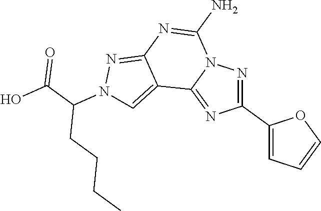 [SMILES: CCCCC(C(=O)O)n1cc2c(nc(N)n3nc(-c4ccco4)nc23)n1]

NaOH solution (160 mg, in 2 mL of water) was added to a solution of methyl 2-(5-amino-2-(furan-2-yl)-8H-pyrazolo[4,3-e][1,2,4]triazolo[1,5-c]pyrimidin-8-yl)hexanoate (380 mg, 1.0 mmol) in methanol (10 mL). The reaction mixture was stirred at rt overnight. The solution was concentrated, added with water (10 mL) and acidified with 1N HCl solution to pH=5. The precipitated solid was filtered and dried to get the desired product as a white solid (355 mg, 97%). MS: M/e 356 (M+1)⁺

### Step B: 2-(5-amino-2-(furan-2-yl)-8H-pyrazolo[4,3-e][1,2,4]triazolo[1,5-c]pyrimidin-8-yl)-1-(4-(4-(2-methoxyethoxy)phenyl)piperazin-1-yl)hexan-1-one

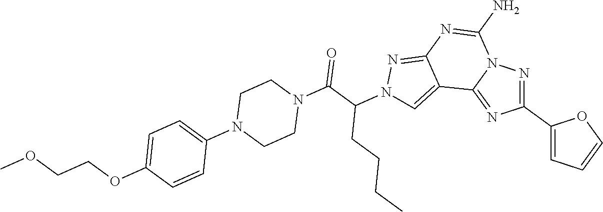 [SMILES: CCCCC(C(=O)N1CCN(c2ccc(OCCOC)cc2)CC1)n1cc2c(nc(N)n3nc(-c4ccco4)nc23)n1]

A mixture of 2-(5-amino-2-(furan-2-yl)-8H-pyrazolo[4,3-e][1,2,4]triazolo[1,5-c]pyrimidin-8-yl)hexanoic acid (50 mg, 0.14 mmol), 1-(4-(2-methoxyethoxy)phenyl)piperazine (33 mg, 0.14 mmol), HATU (64 mg, 0.17 mmol) and DIEA (36 mg, 0.28 mmol) in DMF (5 mL) was stirred at rt for 2 hrs. The solution was added with water (10 mL), extracted with ethyl acetate (10 mL) and washed with brine (10 mL). The organic layer was dried, concentrated and purified by column chromatography (PE:EA=1:1 to EA) to get the desired product as a white solid (16 mg, 20%). ¹H NMR (400 MHz, DMSO-d6) δ 8.74 (s, 1H), 7.94 (d, J=4.0 Hz, 1H), 7.65 (br.s, 2H), 7.18 (d, J=4.0 Hz, 1H), 6.87-6.73 (m, 5H), 5.67 (dd, J=8.0 Hz, 4.0 Hz, 1H), 4.02-3.98 (m, 2H), 3.75-3.60 (m, 6H), 3.29 (s, 3H), 3.00-2.89 (m, 4H), 2.19-2.03 (m, 2H), 1.35-1.13 (m, 4H), 0.85 (t, J=8.0 Hz, 3H) ppm. MS: M/e 574 (M+1)⁺

### Compound C11: 2-(5-amino-2-(furan-2-yl)-8H-pyrazolo[4,3-e][1,2,4]triazolo[1,5-c]pyrimidin-8-yl)-1-(4-(4-(2-(2-methoxyethoxy)ethoxy)phenyl)piperazin-1-yl)hexan-1-one

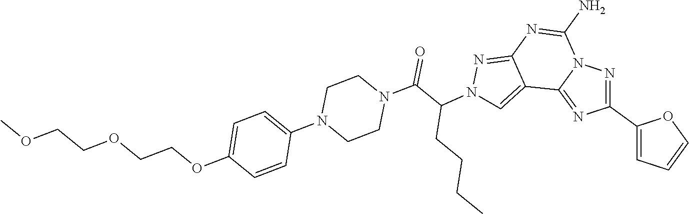 [SMILES: CCCCC(C(=O)N1CCN(c2ccc(OCCOCCOC)cc2)CC1)n1cc2c(nc(N)n3nc(-c4ccco4)nc23)n1]

A mixture of 2-(5-amino-2-(furan-2-yl)-8H-pyrazolo[4,3-e][1,2,4]triazolo[1,5-c]pyrimidin-8-yl)hexanoic acid (50 mg, 0.14 mmol), 1-(4-(2-(2-methoxyethoxy)ethoxy)phenyl)piperazine (39 mg, 0.14 mmol), HATU (64 mg, 0.17 mmol) and DIEA (36 mg, 0.28 mmol) in DMF (10 mL) was stirred at rt for 2 hrs. The solution was added with water (10 mL), extracted with ethyl acetate (10 mL) and washed with brine (10 mL). The organic layer was dried, concentrated and purified by column chromatography (PE:EA=1:1 to EA) to get the desired product (25 mg, 29%). ¹H NMR (400 MHz, DMSO-d6) δ 8.74 (s, 1H), 7.94 (s, 1H), 7.65 (br.s, 2H), 7.18 (d, J=4.0 Hz, 1H), 6.88-6.73 (m, 5H), 5.67 (dd, J=8.0 Hz, 4.0 Hz, 1H), 3.99 (t, J=4.0 Hz, 2H), 3.76-3.55 (m, 8H), 3.46-3.43 (m, 5H), 3.06-2.90 (m, 4H), 2.19-2.06 (m, 2H), 1.35-1.13 (m, 4H), 0.85 (t, J=8.0 Hz, 3H) ppm. MS: M/e 618 (M+1)⁺

### Compound C12: 2-(5-amino-2-(furan-2-yl)-8H-pyrazolo[4,3-e][1,2,4]triazolo[1,5-c]pyrimidin-8-yl)-1-(4-(4-(2-(2-methoxyethoxy)ethoxy)phenyl)piperazin-1-yl)-3-methylbutan-1-one

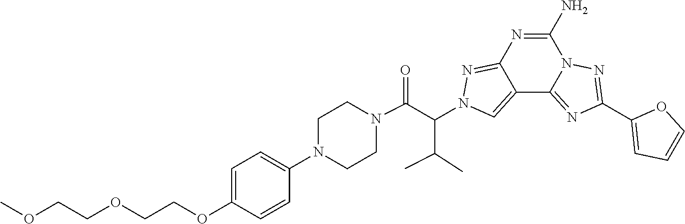 [SMILES: COCCOCCOc1ccc(N2CCN(C(=O)C(C(C)C)n3cc4c(nc(N)n5nc(-c6ccco6)nc45)n3)CC2)cc1]

To a mixture of 2-(5-amino-2-(furan-2-yl)-8H-pyrazolo[4,3-e][1,2,4]triazolo[1,5-c]pyrimidin-8-yl)-3-methylbutanoic acid (55 mg, 0.16 mmol), 1-(4-(2-(2-methoxyethoxy)ethoxy)phenyl)piperazine (50 mg, 0.18 mmol), DIEA (100 mg, 0.77 mmol) in DMF (2 mL) was added HATU (68 mg, 0.18 mmol) at rt and the mixture was stirred at rt for 4 hrs. 20 mL of EA was added and the mixture was washed with brine (10 mL×3), dried over Na₂SO₄ and concentrated. The resulting residue was purified by prep-TLC (EA/MeOH=20:1) to give the title product (35.0 mg, yield: 36%). ¹H NMR (400 MHz, DMSO-d6) δ 8.68 (s, 1H), 7.94 (d, J=1.2 Hz, 1H), 7.65 (s, 2H), 7.19 (d, J=3.6 Hz, 1H), 6.92-6.77 (m, 4H), 6.73 (dd, J=3.2, 2.0 Hz, 1H), 5.40 (d, J=10.4 Hz, 1H), 4.04-3.96 (m, 2H), 3.95-3.85 (m, 1H), 3.80-3.65 (m, 4H), 3.64-3.52 (m, 3H), 3.44-3.42 (m, 2H), 3.23 (s, 3H), 3.03-2.90 (m, 3H), 2.85-2.78 (m, 1H), 2.72-2.62 (m, 1H), 1.01 (d, J=6.4 Hz, 3H), 0.75 (d, J=6.4 Hz, 3H). MS: M/e 604 (M+1)⁺.

### Compound C13: 2-(5-amino-2-(furan-2-yl)-8H-pyrazolo[4,3-e][1,2,4]triazolo[1,5-c]pyrimidin-8-yl)-1-(4-(4-(2-aminoethoxy)phenyl)piperazin-1-yl)propan-1-one

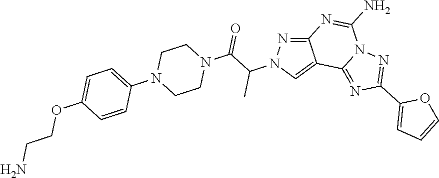 [SMILES: CC(C(=O)N1CCN(c2ccc(OCCN)cc2)CC1)n1cc2c(nc(N)n3nc(-c4ccco4)nc23)n1]

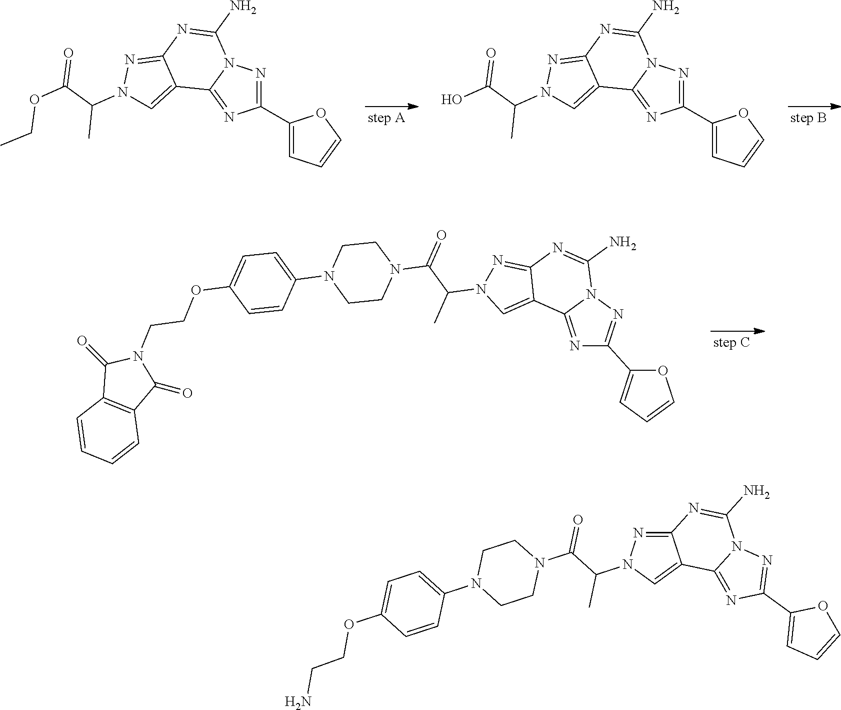 [SMILES: CCOC(=O)C(C)n1cc2c(nc(N)n3nc(-c4ccco4)nc23)n1 ; CC(C(=O)O)n1cc2c(nc(N)n3nc(-c4ccco4)nc23)n1 ; CC(C(=O)N1CCN(c2ccc(OCCN3C(=O)c4ccccc4C3=O)cc2)CC1)n1cc2c(nc(N)n3nc(-c4ccco4)nc23)n1 ; CC(C(=O)N1CCN(c2ccc(OCCN)cc2)CC1)n1cc2c(nc(N)n3nc(-c4ccco4)nc23)n1]

### Step A: 2-(5-amino-2-(furan-2-yl)-8H-pyrazolo[4,3-e][1,2,4]triazolo[1,5-c]pyrimidin-8-yl)propanoic Acid

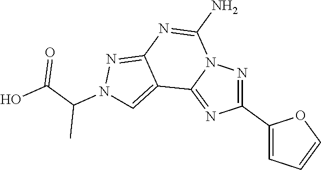 [SMILES: CC(C(=O)O)n1cc2c(nc(N)n3nc(-c4ccco4)nc23)n1]

To a stirred solution of ethyl 2-(5-amino-2-(furan-2-yl)-8H-pyrazolo[4,3-e][1,2,4]triazolo[1,5-c]pyrimidin-8-yl)propanoate (800 g, 2.35 mmol) in MeOH (20 mL) was added aq.NaOH (2.0 M, 6.4 mL) at RT. The mixture was stirred at RT overnight. The solvents were removed and the residue was dissolved into water (20 mL). The mixture was acidified to pH=3˜4 with aq.HCl (2 M). The solid was precipitated from the system. The mixture was filtered and the solid was collected. The white solid (450 mg, 61.3%) was dried in air and used into next step directly. MS: M/e 314 (M+1)⁺.

### Step B: 2-(2-(4-(4-(2-(5-amino-2-(furan-2-yl)-8H-pyrazolo[4,3-e][1,2,4]triazolo[1,5-c]pyrimidin-8-yl)propanoyl)piperazin-1-yl)phenoxy)ethyl)isoindoline-1,3-dione

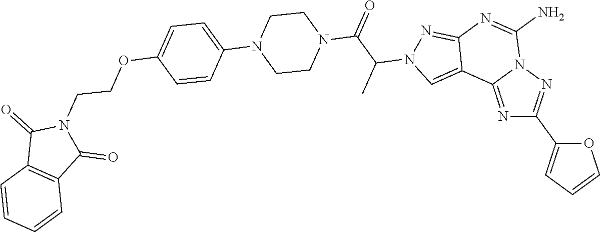 [SMILES: CC(C(=O)N1CCN(c2ccc(OCCN3C(=O)c4ccccc4C3=O)cc2)CC1)n1cc2c(nc(N)n3nc(-c4ccco4)nc23)n1]

A mixture of the product of Step A (150 mg, 0.48 mmol), 2-(2-(4-(piperazin-1-yl)phenoxy)ethyl)isoindoline-1,3-dione (185 mg, 0.53 mmol), HATU (200 mg, 0.53 mmol) and DIEA (0.5 mL, excess) in DMF (10 mL) was stirred at RT overnight. The reaction mixture was poured into water (20 mL) and the solid was precipitated from the system. The solid was filtered and dried in air. The yellow solid (200 mg, yield: 64.5%) was used into next step without further purification. MS: M/e 647 (M+1)⁺.

### Step C: 2-(5-amino-2-(furan-2-yl)-8H-pyrazolo[4,3-e][1,2,4]triazolo[1,5-c]pyrimidin-8-yl)-1-(4-(4-(2-aminoethoxy)phenyl)piperazin-1-yl)propan-1-one

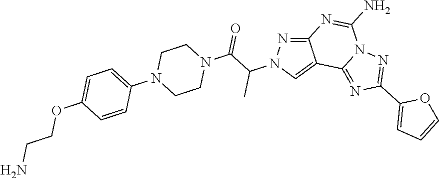 [SMILES: CC(C(=O)N1CCN(c2ccc(OCCN)cc2)CC1)n1cc2c(nc(N)n3nc(-c4ccco4)nc23)n1]

To a stirred solution of the product of Step B in EtOH (30 mL) was added hydrazine hydrate (2 mL) at RT. The mixture was stirred at reflux overnight. The brown solid was precipitated from the system. The mixture was filtered and the solid was collected. The solid was dissolved into EtOH and the mixture was stirred at reflux overnight. The mixture was filtered and the solid was dried in air. The title compound (55 mg, yield: 34.4%) was obtained as brown solid. ¹H NMR (400 MHz, DMSO-d6) δ 8.74 (s, 1H), 7.94 (s, 1H), 7.65 (s, 2H), 7.19 (d, J=4 Hz, 1H), 6.90 (d, J=12 Hz, 2H), 6.82 (d, J=12 Hz, 2H), 6.73 (dd, J=4, 2 Hz, 1H), 5.99-5.71 (m, 1H), 3.83 (t, J=6 Hz, 2H), 3.78-3.67 (m, 2H), 3.68-3.56 (m, 2H), 3.12-2.88 (m, 4H), 2.83 (t, J=6 Hz, 2H), 1.70 (d, J=8 Hz, 3H) ppm. MS: M/e 517 (M+1)⁺.

### Compound C14: 2-(5-amino-2-(furan-2-yl)-8H-pyrazolo[4,3-e][1,2,4]triazolo[1,5-c]pyrimidin-8-yl)-1-(4-(4-(2-methoxyethoxy)phenyl)piperazin-1-yl)-2-phenylethan-1-one

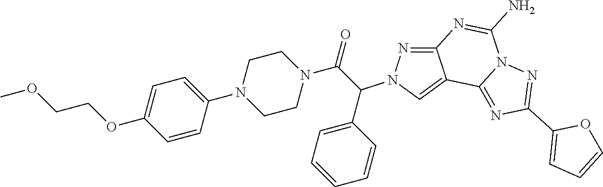 [SMILES: COCCOc1ccc(N2CCN(C(=O)C(c3ccccc3)n3cc4c(nc(N)n5nc(-c6ccco6)nc45)n3)CC2)cc1]

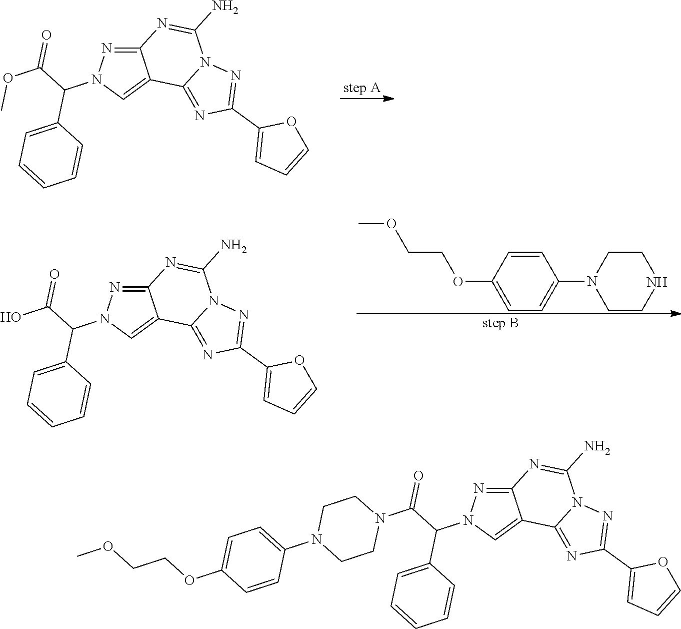

### Step A: 2-(5-amino-2-(furan-2-yl)-8H-pyrazolo[4,3-e][1,2,4]triazolo[1,5-c]pyrimidin-8-yl)-2-phenylacetic Acid

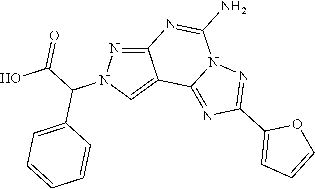 [SMILES: Nc1nc2nn(C(C(=O)O)c3ccccc3)cc2c2nc(-c3ccco3)nn12]

To a stirred mixture of methyl 2-(5-amino-2-(furan-2-yl)-8H-pyrazolo[4,3-e][1,2,4]triazolo[1,5-c]pyrimidin-8-yl)-2-phenylacetate (360 mg, 0.92 mmol) in MeOH/H₂O (9 mL/3 mL) was added aq.NaOH (2.0 M, 2 mL). After the addition, the reaction mixture was stirred overnight. Most of solvent was removed to give the aqueous layer, then acidified to pH=3˜4 with aq.HCl and filtered, the cake was collected, dried to give the target compound (200 mg, 57.8%) as a white solid. MS: M/e 376 (M+1)⁺.

### Step B: 2-(5-amino-2-(furan-2-yl)-8H-pyrazolo[4,3-e][1,2,4]triazolo[1,5-c]pyrimidin-8-yl)-1-(4-(4-(2-methoxyethoxy)phenyl)piperazin-1-yl)-2-phenylethan-1-one

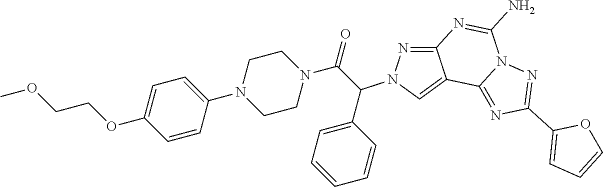 [SMILES: COCCOc1ccc(N2CCN(C(=O)C(c3ccccc3)n3cc4c(nc(N)n5nc(-c6ccco6)nc45)n3)CC2)cc1]

A mixture of the product of step A (50 mg, 0.13 mmol), 1-(4-(2-methoxyethoxy)phenyl)piperazine (55 mg, 0.23 mmol), HATU (75 mg, 0.20 mmol) and TEA (40 mg, 0.39 mmol) in DMF (10 mL) was stirred for 5 hours at RT. The reaction mixture was poured into H₂O (20 mL) and extracted with EtOAc (50 mL×3). The combined organic layers were washed with brine, dried over Na₂SO₄, concentrated and purified by column chromatography (petroleum ether/EtOAc=1:1˜100% EtOAc) to give the target compound (40 mg, 50.7%) as a white solid. ¹H NMR (400 MHz, DMSO-d6) δ 8.17 (s, 1H), 7.92 (s, 1H), 7.70 (br., 2H), 7.59-7.43 (m, 5H), 7.15 (d, J=3.4 Hz, 1H), 7.11 (s, 1H), 6.86-6.77 (m, 4H), 6.75-6.69 (m, 1H), 4.03-3.95 (m, 2H), 3.80-3.71 (m, 1H), 3.71-3.57 (m, 4H), 3.50-3.40 (m, 1H), 3.34 (s, 1H), 3.28 (s, 3H), 3.10-3.01 (m, 1H), 2.98-2.87 (m, 2H) ppm. MS: M/e 594 (M+1)⁺.

### Compound C15: 2-(5-amino-2-(furan-2-yl)-8H-pyrazolo[4,3-e][1,2,4]triazolo[1,5-c]pyrimidin-8-yl)-1-(4-(4-(2-(2-methoxyethoxy)ethoxy)phenyl)piperazin-1-yl)-2-phenylethan-1-one

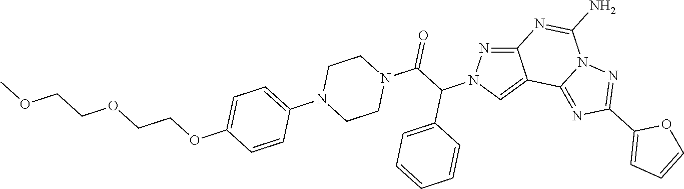 [SMILES: COCCOCCOc1ccc(N2CCN(C(=O)C(c3ccccc3)n3cc4c(nc(N)n5nc(-c6ccco6)nc45)n3)CC2)cc1]

A mixture of 2-(5-amino-2-(furan-2-yl)-8H-pyrazolo[4,3-e][1,2,4]triazolo[1,5-c]pyrimidin-8-yl)-2-phenylacetic acid (50 mg, 0.13 mmol), 1-(4-(2-(2-methoxyethoxy)ethoxy)phenyl)piperazine (40 mg, 0.14 mmol), HATU (84 mg, 0.22 mmol) and TEA (40 mg, 0.39 mmol) in DMF (10 mL) was stirred for 4 hours at RT. The reaction mixture was poured into H₂O (20 mL) and extracted with EtOAc (50 mL×3). The combined organic layers were washed with brine, dried over Na₂SO₄, concentrated and purified by column chromatography (petroleum ether/EtOAc=1:1˜100% EtOAc) to give the target compound (32 mg, 37.8%). ¹H NMR (400 MHz, DMSO-d6) δ 8.17 (s, 1H), 7.93 (s, 1H), 7.77-7.66 (m, 2H), 7.59-7.46 (m, 5H), 7.15 (d, J=3.3 Hz, 1H), 7.11 (s, 1H), 6.86-6.78 (m, 5H), 6.75-6.69 (m, 1H), 4.02-3.96 (m, 2H), 3.80-3.61 (m, 5H), 3.60-3.54 (m, 2H), 3.50-3.41 (m, 3H), 3.24 (s, 3H), 3.09-2.98 (m, 2H), 2.98-2.88 (m, 2H) ppm. MS: M/e 638 (M+1)⁺.

### Compound C16: 2-(5-amino-2-(furan-2-yl)-8H-pyrazolo[4,3-e][1,2,4]triazolo[1,5-c]pyrimidin-8-yl)-2-cyclopropyl-1-(4-(4-(2-methoxyethoxy)phenyl)piperazin-1-yl)ethan-1-one

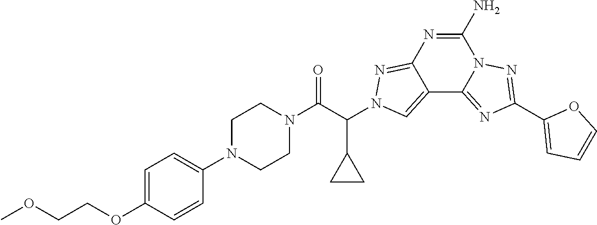 [SMILES: COCCOc1ccc(N2CCN(C(=O)C(C3CC3)n3cc4c(nc(N)n5nc(-c6ccco6)nc45)n3)CC2)cc1]

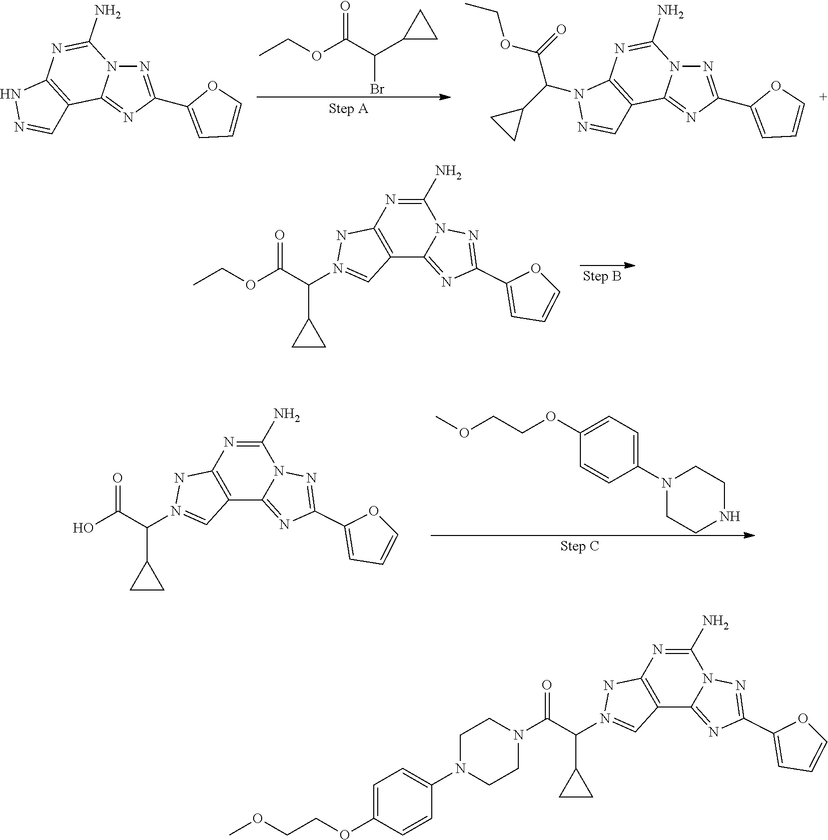

### Step A: ethyl 2-(5-amino-2-(furan-2-yl)-7H-pyrazolo[4,3-e][1,2,4]triazolo[1,5-c]pyrimidin-7-yl)-2-cyclopropylacetate and ethyl 2-(5-amino-2-(furan-2-yl)-8H-pyrazolo[4,3-e][1,2,4]triazolo[1,5-c]pyrimidin-8-yl)-2-cyclopropylacetate

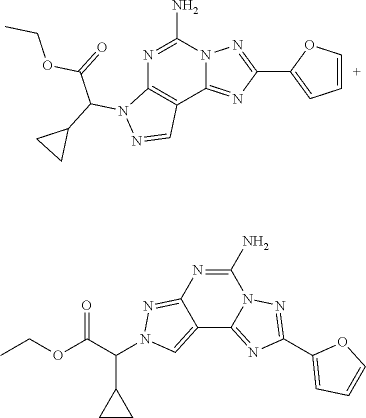 [SMILES: CCOC(=O)C(C1CC1)n1ncc2c1nc(N)n1nc(-c3ccco3)nc21 ; CCOC(=O)C(C1CC1)n1cc2c(nc(N)n3nc(-c4ccco4)nc23)n1]

A mixture of 2-(furan-2-yl)-7H-pyrazolo[4,3-e][1,2,4]triazolo[1,5-c]pyrimidin-5-amine (5.0 g, 20.7 mmol), ethyl 2-bromo-2-cyclopropylacetate (6.4 g, 31.1 mmol) and K₂CO₃ (7.1 g, 51.4 mmol) in DMF (50 mL) was heated at 50° C. for 16 hrs. The mixture was diluted with EA (200 mL) and the suspension was filtered. The filtrate was washed with brine (50 mL×3), dried over Na₂SO₄ and concentrated. The resulting oil was purified by column chromatography eluted with PE/EA (5:1˜2:1) to give ethyl 2-(5-amino-2-(furan-2-yl)-7H-pyrazolo[4,3-e][1,2,4]triazolo[1,5-c]pyrimidin-7-yl)-2-cyclopropylacetate (1.4 g) as a light yellow solid, MS: M/e 368 (M+1)⁺, and ethyl 2-(5-amino-2-(furan-2-yl)-8H-pyrazolo[4,3-e][1,2,4]triazolo[1,5-c]pyrimidin-8-yl)-2-cyclopropylacetate (1.9 g) as a light yellow solid, MS: M/e 368 (M+1)⁺.

### Step B: 2-(5-amino-2-(furan-2-yl)-8H-pyrazolo[4,3-e][1,2,4]triazolo[1,5-c]pyrimidin-8-yl)-2-cyclopropylacetic Acid

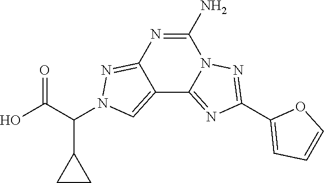 [SMILES: Nc1nc2nn(C(C(=O)O)C3CC3)cc2c2nc(-c3ccco3)nn12]

To a stirred solution of ethyl 2-(5-amino-2-(furan-2-yl)-8H-pyrazolo[4,3-e][1,2,4]triazolo[1,5-c]pyrimidin-8-yl)-2-cyclopropylacetate (1.8 g, 4.9 mmol) in MeOH (30 mL) was added aqueous solution of NaOH (1 M, 15 mL) at rt and the resulting mixture was stirred for 4 hrs. The mixture was neutralized by HCl (1 M) to pH=6. A white solid precipitated and which was filtered and the filter cake was dried under IR lamp to give the title product (1.4 g, 84%) as a white solid. MS: M/e340 (M+1)⁺.

### Step C: 2-(5-amino-2-(furan-2-yl)-8H-pyrazolo[4,3-e][1,2,4]triazolo[1,5-c]pyrimidin-8-yl)-2-cyclopropyl-1-(4-(4-(2-methoxyethoxy)phenyl)piperazin-1-yl)ethan-1-one

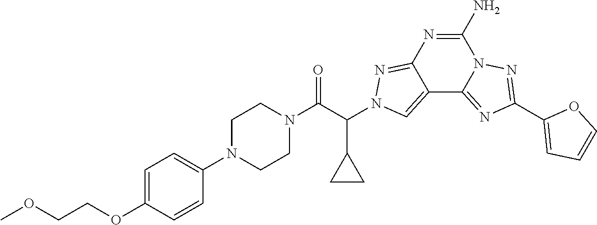 [SMILES: COCCOc1ccc(N2CCN(C(=O)C(C3CC3)n3cc4c(nc(N)n5nc(-c6ccco6)nc45)n3)CC2)cc1]

To a mixture of the product from step B (600 mg, 1.77 mmol), 1-(4-(2-methoxyethoxy)phenyl)piperazine (460 mg, 1.95 mmol), DIEA (550 mg, 4.26 mmol) in DMF (10 mL) was added HATU (750 mg, 2.0 mmol) at rt and the mixture was stirred at rt for 14 hrs. The mixture was poured into 40 mL of H₂O and the white precipitate was filtered. The filtered cake was purified by column chromatography (DCM/MeOH=50:1˜30:1) to give the title product (428 mg, 78%). ¹H NMR (400 MHz, DMSO-d6) δ 8.83 (s, 1H), 7.94 (s, 1H), 7.64 (s, 2H), 7.19 (d, J=3.2 Hz, 1H), 6.87 (dd, J=28.4, 8.4 Hz, 4H), 6.74 (s, 1H), 5.13 (d, J=9.6 Hz, 1H), 4.08-3.96 (m, 2H), 3.89-3.71 (m, 2H), 3.70-3.55 (m, 4H), 3.29 (s, 3H), 3.07-2.91 (m, 4H), 1.84-1.63 (m, 1H), 0.80-0.62 (m, 3H), 0.53-0.38 (m, 1H). MS: M/e 558 (M+1)⁺.

Compound C16 was separated into two enantiomeric stereoisomers, Compound C16a (earlier peak), and Compound C16b (later peak) by chiral prep-HPLC. The chiral separation conditions are shown below.

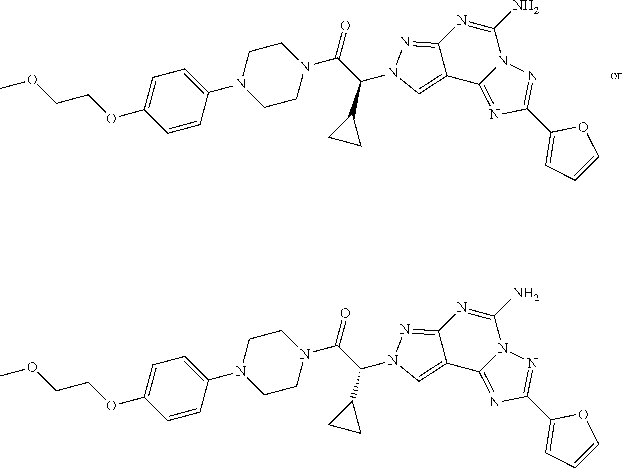 [SMILES: COCCOc1ccc(N2CCN(C(=O)[C@H](C3CC3)n3cc4c(nc(N)n5nc(-c6ccco6)nc45)n3)CC2)cc1 ; COCCOc1ccc(N2CCN(C(=O)[C@@H](C3CC3)n3cc4c(nc(N)n5nc(-c6ccco6)nc45)n3)CC2)cc1]

| Column | CHIRALPAK Cellulose-IC |
|---|---|
| Column size | 2 cm × 25 cm, 5 um |
| Injection | 0.8 mL |
| Mobile phase | MeOH:DCM = 70:30 |
| Flow rate | 18 ml/min |
| Wavelength | UV 220 nm |
| Temperature | 25° C. |
| Sample solution | 25.4 mg/ml in DCM:EtOH = 1:1 |
| Prep-HPLC equipment | BJ-Prep-Gilson-HPLC |

### Compound C17: 2-(5-amino-2-(furan-2-yl)-8H-pyrazolo[4,3-e][1,2,4]triazolo[1,5-c]pyrimidin-8-yl)-1-(4-(4-(2-methoxyethoxy)phenyl)piperazin-1-yl)-4-methylpentan-1-one

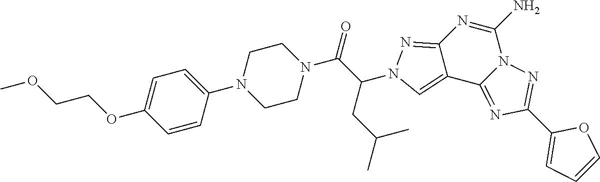 [SMILES: COCCOc1ccc(N2CCN(C(=O)C(CC(C)C)n3cc4c(nc(N)n5nc(-c6ccco6)nc45)n3)CC2)cc1]

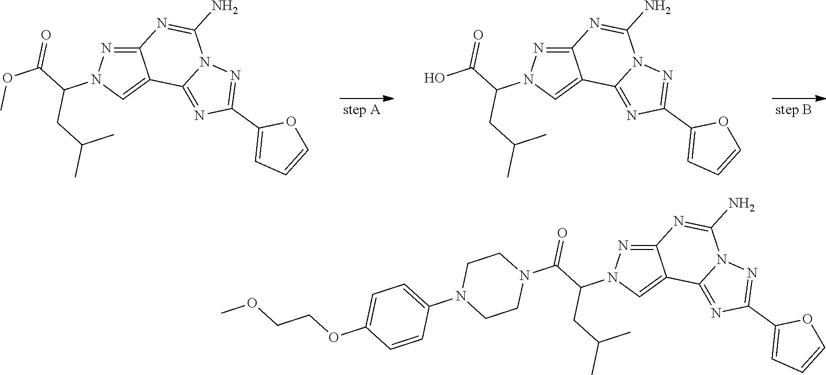

### Step A: 2-(5-amino-2-(furan-2-yl)-8H-pyrazolo[4,3-e][1,2,4]triazolo[1,5-c]pyrimidin-8-yl)-4-methylpentanoic Acid

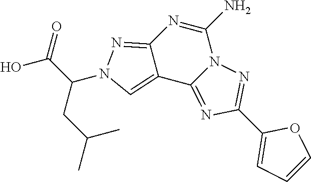 [SMILES: CC(C)CC(C(=O)O)n1cc2c(nc(N)n3nc(-c4ccco4)nc23)n1]

To a stirred mixture of methyl 2-(5-amino-2-(furan-2-yl)-8H-pyrazolo[4,3-e][1,2,4]triazolo[1,5-c]pyrimidin-8-yl)-4-methylpentanoate (270 mg, 0.73 mmol) in MeOH/H₂O (10 mL/5 mL) was added aq.NaOH (2.0 M, 4 mL). After the addition, the reaction mixture was stirred overnight. Most of solvent was removed to give the aqueous layer, then acidified to pH=3˜4 with aq.HCl and filtered, the cake was collected, dried to give the target compound (200 mg, 77%) as a white solid. MS: M/e 356 (M+1)⁺.

### Step B: 2-(5-amino-2-(furan-2-yl)-8H-pyrazolo[4,3-e][1,2,4]triazolo[1,5-c]pyrimidin-8-yl)-1-(4-(4-(2-methoxyethoxy)phenyl)piperazin-1-yl)-4-methylpentan-1-one

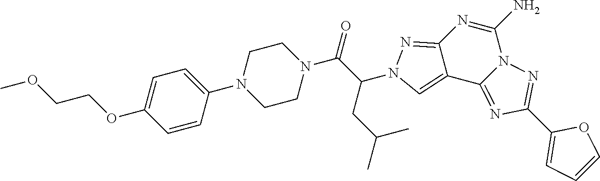 [SMILES: COCCOc1ccc(N2CCN(C(=O)C(CC(C)C)n3cc4c(nc(N)n5nc(-c6ccco6)nc45)n3)CC2)cc1]

A mixture of the product of step A (50 mg, 0.14 mmol), 1-(4-(2-methoxyethoxy)phenyl)piperazine (33 mg, 0.14 mmol), HATU (64 mg, 0.168 mmol) and DIPEA (36 mg, 0.28 mmol) in DMF (5 mL) was stirred for 2 hours at RT. The reaction mixture was poured into H₂O (20 mL) and extracted with EtOAc (20 mL×3). The combined organic layers were washed with brine, dried over Na₂SO₄, concentrated and purified by column chromatography (petroleum ether/EtOAc=1:1˜100% EtOAc) to give the target compound (20 mg, 24.9%). ¹H NMR (400 MHz, DMSO-d6) δ 8.77 (s, 1H), 7.94 (d, J=0.8 Hz, 1H), 7.65 (s, 2H), 7.26-7.10 (m, 1H), 6.85 (dd, J=25.6, 9.2 Hz, 4H), 6.74 (dd, J=3.2, 1.6 Hz, 1H), 5.70 (m, 1H), 4.05-3.95 (m, 2H), 3.74 (s, 2H), 3.67-3.53 (m, 4H), 3.30 (s, 3H), 3.13-2.96 (m, 2H), 2.91 (m, 2H), 2.28-2.15 (m, 1H), 1.91-1.77 (m, 1H), 1.43-1.27 (m, 1H), 0.96 (d, J=6.4 Hz, 3H), 0.88 (d, J=6.4 Hz, 3H) ppm. MS: M/e 574 (M+1)⁺.

### Compound C18: 2-(5-amino-2-(furan-2-yl)-8H-pyrazolo[4,3-e][1,2,4]triazolo[1,5-c]pyrimidin-8-yl)-1-(4-(4-(2-(2-methoxyethoxy)ethoxy)phenyl)piperazin-1-yl)-4-methylpentan-1-one

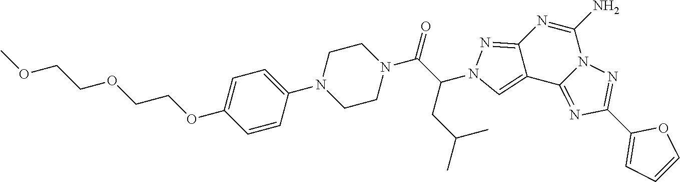 [SMILES: COCCOCCOc1ccc(N2CCN(C(=O)C(CC(C)C)n3cc4c(nc(N)n5nc(-c6ccco6)nc45)n3)CC2)cc1]

A mixture of 2-(5-amino-2-(furan-2-yl)-8H-pyrazolo[4,3-e][1,2,4]triazolo[1,5-c]pyrimidin-8-yl)-4-methylpentanoic acid (50 mg, 0.14 mmol), 1-(4-(2-(2-methoxyethoxy)ethoxy)phenyl)piperazine (39.4 mg, 0.14 mmol), HATU (64 mg, 0.168 mmol) and DIPEA (36 mg, 0.28 mmol) in DMF (5 mL) was stirred for 2 hours at RT. The reaction mixture was poured into H₂O (20 mL) and extracted with EtOAc (20 mL×3). The combined organic layers were washed with brine, dried over Na₂SO₄, concentrated and purified by prep-HPLC to give the target compound as TFA salt, which was treated with aq.K₂CO₃ and extracted with EtOAc (15 mL×2). The combined organic layers were washed with brine, dried over Na₂SO₄ and concentrated to give the target compound (10 mg, 11.6%). ¹H NMR (400 MHz, DMSO-d6) δ 8.77 (s, 1H), 7.94 (s, 1H), 7.65 (s, 2H), 7.18 (d, J=3.2 Hz, 1H), 6.85 (dd, J=25.2, 9.2 Hz, 4H), 6.74 (dd, J=3.2, 1.6 Hz, 1H), 5.70 (dd, J=10.4, 4.8 Hz, 1H), 4.36 (t, J=5.2 Hz, 1H), 4.04-3.96 (m, 2H), 3.81-3.52 (m, 8H), 3.45 (m, 2H), 3.24 (s, 3H), 3.13-2.84 (m, 4H), 2.22 (m, 1H), 1.83 (m, 1H), 1.35 (m, 1H), 0.97 (d, J=6.4 Hz, 3H), 0.87 (d, J=6.4 Hz, 3H) ppm. MS: M/e 618 (M+1)⁺.

### Compound C19: 2-(5-amino-2-(furan-2-yl)-8H-pyrazolo[4,3-e][1,2,4]triazolo[1,5-c]pyrimidin-8-yl)-1-(4-(4-(2-(dimethylamino)ethoxy)phenyl)piperazin-1-yl)propan-1-one

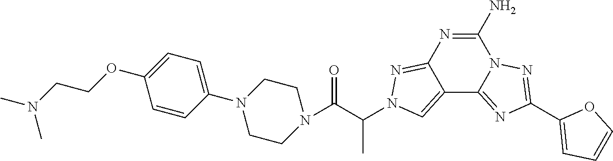 [SMILES: CC(C(=O)N1CCN(c2ccc(OCCN(C)C)cc2)CC1)n1cc2c(nc(N)n3nc(-c4ccco4)nc23)n1]

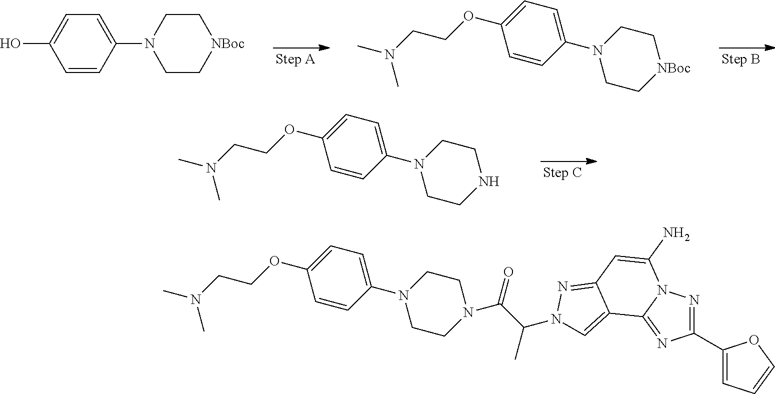

### Step A: tert-butyl 4-(4-(2-(dimethylamino)ethoxy)phenyl)piperazine-1-carboxylate

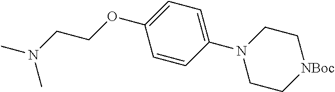 [SMILES: CN(C)CCOc1ccc(N2CCN(C(=O)OC(C)(C)C)CC2)cc1]

To a stirred solution of tert-butyl 4-(4-hydroxyphenyl)piperazine-1-carboxylate (2 g, 7.2 mmol) in DMF (20 mL) was added NaH (0.36 g, 9.4 mmol) at 0° C. The mixture was stirred at 0° C. for 30 mins. Then 2-chloro-N,N-dimethylethan-1-amine (1 g, 9.4 mmol) was added to the reaction. The reaction was stirred at RT overnight. The reaction was quenched with water (20 mL) and extracted with DCM (20 mL×3). The combined organic phase was washed with brine, dried over Na₂SO₄ and concentrated under reduced pressure. The residue (crude) was used into next step directly. MS: M/e 350 (M+1)⁺.

### Step B: N,N-dimethyl-2-(4-(piperazin-1-yl)phenoxy)ethan-1-amine Hydrochloride

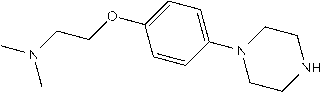 [SMILES: CN(C)CCOc1ccc(N2CCNCC2)cc1]

The crude product of Step A was dissolved into HCl/1,4-dioxane (4M, 20 mL) at RT. The mixture was stirred at RT for 4 hours. The brown solid was precipitated from the system. The mixture was filtered and the solid was collected. The solid (500 mg, yield: 27.9%) was dried in air and used into next step directly. MS: M/e 250 (M+1)⁺.

### Step C: 2-(5-amino-2-(furan-2-yl)-8H-pyrazolo[4,3-e][1,2,4]triazolo[1,5-c]pyrimidin-8-yl)-1-(4-(4-(2-(dimethylamino)ethoxy)phenyl)piperazin-1-yl)propan-1-one

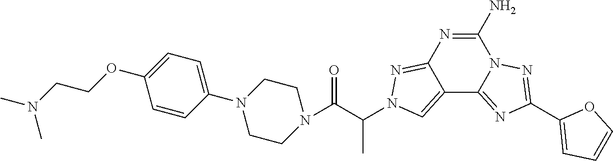 [SMILES: CC(C(=O)N1CCN(c2ccc(OCCN(C)C)cc2)CC1)n1cc2c(nc(N)n3nc(-c4ccco4)nc23)n1]

A mixture of the product of Step B (50 mg, 0.18 mmol), 2-(5-amino-2-(furan-2-yl)-8H-pyrazolo[4,3-e][1,2,4]triazolo[1,5-c]pyrimidin-8-yl)propanoic acid (50 mg, 0.16 mmol), HATU (67 mg, 0.18 mmol) and DIEA (0.5 mL, excess) in DMF (5 mL) was stirred at RT overnight. The reaction mixture was poured into water (10 mL) and the solid was precipitated from the system. The solid was filtered and purified by prep-HPLC to afford the title compound (8 mg, yield: 9.8%). ¹H NMR (400 MHz, DMSO-d6) δ 8.82 (s, 1H), 7.96 (s, 1H), 7.89-7.68 (m, 2H), 7.63-7.35 (m, 2H), 7.20 (d, J=4 Hz, 1H), 7.10 (d, J=8 Hz, 2H), 6.74 (s, 1H), 5.91 (d, J=8 Hz, 1H), 4.38 (s, 2H), 3.96-3.78 (m, 6H), 3.57-3.43 (m, 4H), 2.90-2.72 (m, 6H), 1.81-1.67 (m, 3H) ppm. MS: M/e 545 (M+1)⁺.

### Compound C20: 2-(5-amino-2-(furan-2-yl)-8H-pyrazolo[4,3-e][1,2,4]triazolo[1,5-c]pyrimidin-8-yl)-1-(4-(4-(2-methoxyethoxy)phenyl)piperazin-1-yl)-3-phenylpropan-1-one

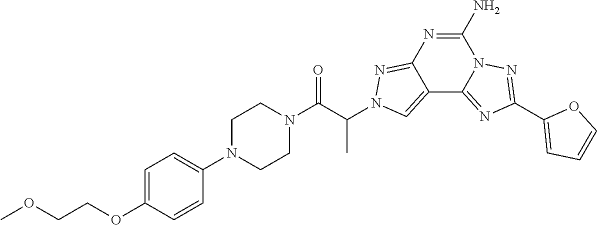 [SMILES: COCCOc1ccc(N2CCN(C(=O)C(C)n3cc4c(nc(N)n5nc(-c6ccco6)nc45)n3)CC2)cc1]

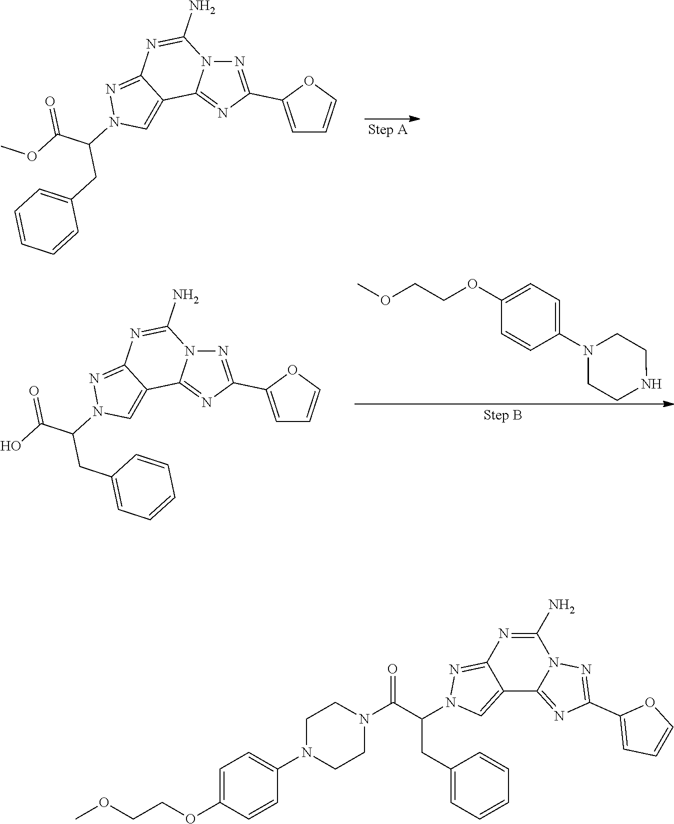 [SMILES: COC(=O)C(Cc1ccccc1)n1cc2c(nc(N)n3nc(-c4ccco4)nc23)n1 ; Nc1nc2nn(C(Cc3ccccc3)C(=O)O)cc2c2nc(-c3ccco3)nn12 ; COCCOc1ccc(N2CCNCC2)cc1 ; COCCOc1ccc(N2CCN(C(=O)C(Cc3ccccc3)n3cc4c(nc(N)n5nc(-c6ccco6)nc45)n3)CC2)cc1]

### Step A: 2-(5-amino-2-(furan-2-yl)-8H-pyrazolo[4,3-e][1,2,4]triazolo[1,5-c]pyrimidin-8-yl)-3-phenylpropanoic Acid

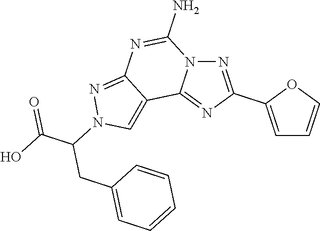 [SMILES: Nc1nc2nn(C(Cc3ccccc3)C(=O)O)cc2c2nc(-c3ccco3)nn12]

To a stirred solution of methyl 2-(5-amino-2-(furan-2-yl)-8H-pyrazolo[4,3-e][1,2,4]triazolo[1,5-c]pyrimidin-8-yl)-3-phenylpropanoate (115 mg, 0.28 mmol) in MeOH (5 mL) was added aqueous solution of NaOH (2 M, 2 mL) at rt and the resulting mixture was stirred for 2 hrs. The mixture was neutralized by HCl (2 M) and concentrated to dryness. 20 mL of a mixed solvent (CH₂Cl₂/MeOH=3:1) was added and stirred for 2 hrs. The suspension was filtered and the filtrate was concentrated to give the title product (90 mg, 81%) as a white solid. MS: M/e 390 (M+1)⁺.

### Step B: 2-(5-amino-2-(furan-2-yl)-8H-pyrazolo[4,3-e][1,2,4]triazolo[1,5-c]pyrimidin-8-yl)-1-(4-(4-(2-methoxyethoxy)phenyl)piperazin-1-yl)-3-phenylpropan-1-one

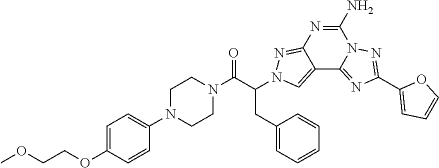 [SMILES: COCCOc1ccc(N2CCN(C(=O)C(Cc3ccccc3)n3cc4c(nc(N)n5nc(-c6ccco6)nc45)n3)CC2)cc1]

To a mixture of the product from step A (45 mg, 0.12 mmol), 1-(4-(2-methoxyethoxy)phenyl)piperazine (45 mg, 0.19 mmol), DIEA (70 mg, 0.54 mmol) in DMF (2 mL) was added HATU (75 mg, 0.20 mmol) at rt and the mixture was stirred at rt for 16 hrs. 30 mL of EA was added and the mixture was washed with brine (15 mL×3), dried over Na₂SO₄ and concentrated. The resulting residue was purified by prep-TLC (CH₂Cl₂/EA/MeOH=10:10:1), and the resulting product was purified by prep-HPLC to give the title product (45 mg, yield: 64%). ¹H NMR (400 MHz, DMSO-d6) δ 8.72 (s, 1H), 7.94 (s, 1H), 7.65 (s, 2H), 7.31-7.20 (m, 4H), 7.20-7.12 (m, 2H), 6.89-6.77 (m, 4H), 6.73 (dd, J=3.2, 1.6 Hz, 1H), 6.01 (t, J=7.2 Hz, 1H), 4.07-3.93 (m, 2H), 3.75-3.58 (m, 5H), 3.58-3.52 (m, 1H), 3.51-3.45 (m, 2H), 3.29 (s, 3H), 3.00-2.89 (m, 1H), 2.89-2.74 (m, 2H), 2.74-2.65 (m, 1H). MS: M/e 608 (M+1)⁺.

### Compound C21: 2-(5-amino-2-(furan-2-yl)-8H-pyrazolo[4,3-e][1,2,4]triazolo[1,5-c]pyrimidin-8-yl)-1-(4-(2,4-difluorophenyl)piperazin-1-yl)pentan-1-one

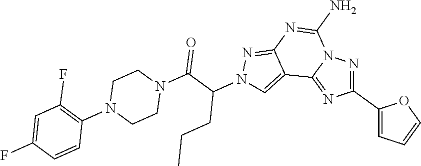 [SMILES: CCCC(C(=O)N1CCN(c2ccc(F)cc2F)CC1)n1cc2c(nc(N)n3nc(-c4ccco4)nc23)n1]

A mixture of 2-(5-amino-2-(furan-2-yl)-8H-pyrazolo[4,3-e][1,2,4]triazolo[1,5-c]pyrimidin-8-yl)pentanoic acid (50 mg, 0.15 mmol), 1-(2,4-difluorophenyl)piperazine (32 mg, 0.16 mmol), HATU (69 mg, 0.18 mmol) and DIEA (39 mg, 0.3 mmol) in DMF (5 mL) was stirred at rt for 2 hrs. The solution was added with water (5 mL), extracted with ethyl acetate (10 mL) and washed with brine (10 mL). The organic layer was dried, concentrated and purified by column chromatography (PE:EA=1:1) to get the desired product (49 mg, 64%). ¹H NMR (400 MHz, DMSO-d6) δ 8.76 (s, 1H), 7.95 (s, 1H), 7.66 (br.s, 2H), 7.19-6.96 (m, 4H), 6.74-6.73 (m, 1H), 5.71-5.67 (m, 1H), 3.78-3.65 (m, 4H), 2.94-2.83 (m, 4H), 2.19-1.99 (m, 2H), 1.33-1.17 (m, 2H), 0.92 (t, J=8.0 Hz, 3H) ppm. MS: M/e 522 (M+1)⁺

### Compound C22: 2-(5-amino-2-(furan-2-yl)-8H-pyrazolo[4,3-e][1,2,4]triazolo[1,5-c]pyrimidin-8-yl)-2-cyclopropyl-1-(4-(4-(2-(2-methoxyethoxy)ethoxy)phenyl)piperazin-1-yl)ethan-1-one

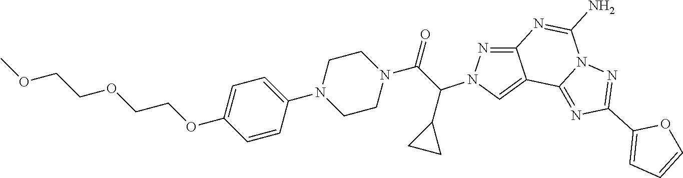 [SMILES: COCCOCCOc1ccc(N2CCN(C(=O)C(C3CC3)n3cc4c(nc(N)n5nc(-c6ccco6)nc45)n3)CC2)cc1]

A mixture of 2-(5-amino-2-(furan-2-yl)-8H-pyrazolo[4,3-e][1,2,4]triazolo[1,5-c]pyrimidin-8-yl)-2-cyclopropylacetic acid (50 mg, 0.15 mmol), 1-(4-(2-(2-methoxyethoxy)ethoxy)phenyl)piperazine (50 mg, 0.18 mmol), HATU (84 mg, 0.22 mmol) and TEA (30 mg, 0.30 mmol) in DMF (10 mL) was stirred for 4 hours at RT. The reaction mixture was poured into H₂O (20 mL) and extracted with EtOAc (50 mL×3). The combined organic layers were washed with brine, dried over Na₂SO₄, concentrated and purified by column chromatography (petroleum ether/EtOAc=1:1˜100% EtOAc) to give the target compound (38 mg, 42.2%). ¹H NMR (400 MHz, DMSO-d6) δ 8.84 (s, 1H), 7.95 (s, 1H), 7.66 (br, 2H), 7.20 (d, J=3.1 Hz, 1H), 6.90 (d, J=9.0 Hz, 2H), 6.83 (d, J=8.9 Hz, 2H), 6.76-6.72 (m, 1H), 5.14 (d, J=9.7 Hz, 1H), 4.04-3.95 (m, 2H), 3.85-3.75 (m, 2H), 3.73-3.63 (m, 5H), 3.60-3.53 (m, 2H), 3.50-3.42 (m, 2H), 3.37-3.31 (m, 1H), 3.28-3.24 (m, 2H), 3.01-2.92 (m, 3H), 1.75 (d, J=3.8 Hz, 1H), 0.78-0.66 (m, 3H), 0.46 (d, J=5.5 Hz, 1H). MS: M/e 602 (M+1)⁺.

### Compound C23: 2-(5-amino-2-(furan-2-yl)-8H-pyrazolo[4,3-e][1,2,4]triazolo[1,5-c]pyrimidin-8-yl)-1-(4-(2,4-difluorophenyl)piperazin-1-yl)-4-methylpentan-1-one

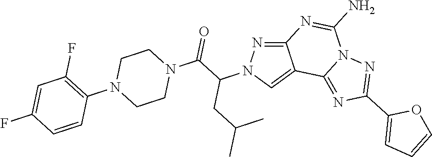 [SMILES: CC(C)CC(C(=O)N1CCN(c2ccc(F)cc2F)CC1)n1cc2c(nc(N)n3nc(-c4ccco4)nc23)n1]

A mixture of the 2-(5-amino-2-(furan-2-yl)-8H-pyrazolo[4,3-e][1,2,4]triazolo[1,5-c]pyrimidin-8-yl)-4-methylpentanoic acid (35.5 mg, 0.1 mmol), 1-(2,4-difluorophenyl)piperazine (19.8 mg, 0.1 mmol), HATU (46 mg, 0.12 mmol) and DIPEA (25.8 mg, 0.2 mmol) in DMF (3 mL) was stirred overnight. The reaction mixture was poured into H₂O (15 mL) and extracted with EtOAc (20 mL×3). The combined organic layers were washed with brine, dried over Na₂SO₄, concentrated and purified by column chromatography (petroleum ether/EtOAc=10:1˜3:1) to give the target compound (20 mg, 37.3%). ¹H NMR (400 MHz, DMSO-d6) δ 8.78 (s, 1H), 7.94 (s, 1H), 7.65 (s, 2H), 7.25-7.17 (m, 2H), 7.03 (m, 2H), 6.74 (dd, J=3.2, 1.6 Hz, 1H), 5.69 (m, 1H), 3.76 (s, 2H), 3.64 (m, 2H), 3.05-2.93 (m, 2H), 2.91-2.79 (m, 2H), 2.28-2.17 (m, 1H), 1.89-1.78 (m, 1H), 1.35 (m, 1H), 0.97 (d, J=6.4 Hz, 3H), 0.88 (d, J=6.4 Hz, 3H) ppm. MS: M/e 536 (M+1)⁺.

### Compound C24: 2-(5-amino-2-(furan-2-yl)-8H-pyrazolo[4,3-e][1,2,4]triazolo[1,5-c]pyrimidin-8-yl)-1-(4-(4-(2-(methylamino)ethoxy)phenyl)piperazin-1-yl)propan-1-one

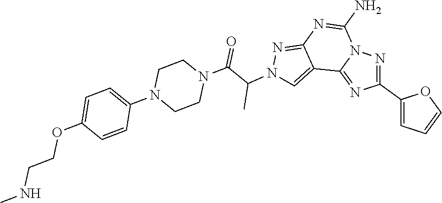 [SMILES: CNCCOc1ccc(N2CCN(C(=O)C(C)n3cc4c(nc(N)n5nc(-c6ccco6)nc45)n3)CC2)cc1]

A mixture of N-methyl-2-(4-(piperazin-1-yl)phenoxy)ethan-1-amine hydrochloride (294 mg, 0.96 mmol), 2-(5-amino-2-(furan-2-yl)-8H-pyrazolo[4,3-e][1,2,4]triazolo[1,5-c]pyrimidin-8-yl)propanoic acid (200 mg, 0.64 mmol), HATU (291 mg, 0.78 mmol) and DIEA (1 mL, excess) in DMF (20 mL) was stirred at RT overnight. The reaction mixture was poured into water (20 mL) and the solid was precipitated from the system. The solid was filtered and purified by prep-HPLC to afford the title compound (50 mg, yield: 14.8%). ¹H NMR (400 MHz, DMSO-d6) δ 9.31 (br.s, 2H), 8.23 (s, 1H), 8.18 (br.s, 1H), 8.01-7.90 (m, 1H), 7.75-7.52 (m, 2H), 7.30-7.20 (m, 1H), 7.16-7.03 (m, 2H), 6.80-6.68 (m, 1H), 5.87-5.69 (m, 1H), 4.29 (t, J=4 Hz, 2H), 4.17-3.72 (m, 4H), 3.64-3.24 (m, 5H), 3.17-2.93 (m, 1H), 2.59 (t, 0.1=4 Hz, 3H), 1.65 (d, J=8 Hz, 3H) ppm. MS: M/e 531 (M+1)⁺.

### Compound C25: 2-(5-amino-2-(furan-2-yl)-8H-pyrazolo[4,3-e][1,2,4]triazolo[1,5-c]pyrimidin-8-yl)-1-(4-(4-(2-methoxyethoxy)phenyl)piperazin-1-yl)-2-phenylpropan-1-one

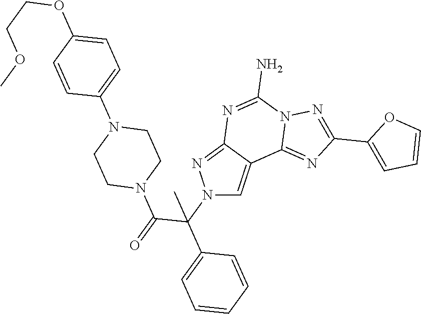 [SMILES: COCCOc1ccc(N2CCN(C(=O)C(C)(c3ccccc3)n3cc4c(nc(N)n5nc(-c6ccco6)nc45)n3)CC2)cc1]

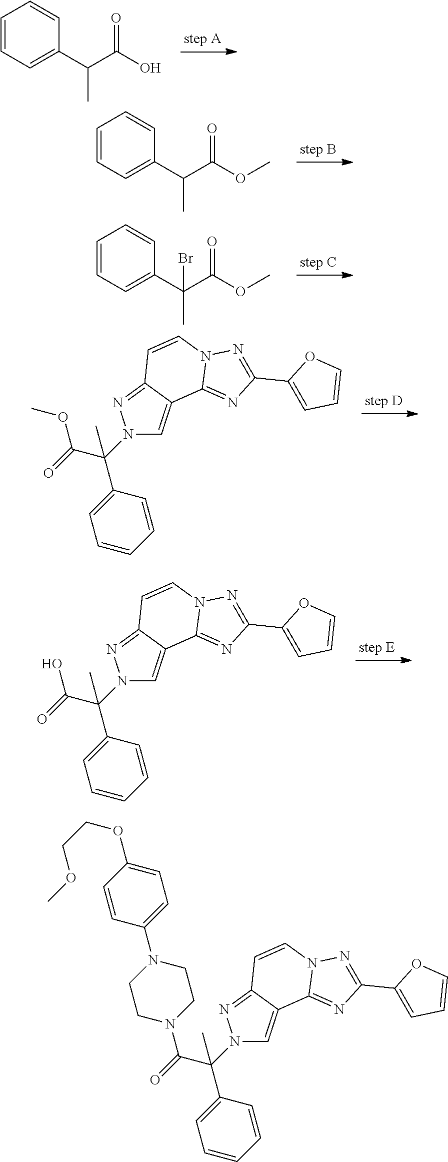

### Step A: methyl 2-phenylpropanoate

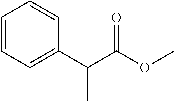

To a solution of 2-phenylpropanoic acid (5 g, 33.33 mmol) in MeOH (15 mL), sulfoxide chloride (5.15 g, 50 mmol) was added dropwise at 0° C., After the addition, the reaction mixture was stirred at rt for 3 h. The mixture was concentrated, quenched with ice water (20 mL), extracted with EtOAc (30 mL×3). The combined organic layers were washed with brine, dried over Na₂SO₄, concentrated and purified by column chromatography (petroleum ether/EtOAc=20:1˜5:1) to give methyl 2-phenylpropanoate (5.22 g, 95.49%) as yellow oil. MS: M/e 165 (M+1)⁺.

### Step B: methyl 2-bromo-2-phenylpropanoate

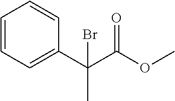

A mixture of methyl 2-phenylpropanoate (5.22 g, 31.83 mmol), NBS (6.80 g, 38.19 mmol), BPO (0.385 g, 1.591 mmol) in carbon tetrachloride (20 mL) was stirred at 70° C. overnight. The mixture was concentrated, the residue was washed with PE and filtered, the filtrate was concentrated to give methyl 2-bromo-2-phenylpropanoate (7.57 g, 97.87%) as yellow oil. ¹H NMR (400 MHz, CDCl₃) δ 7.55 (d, J=7.5 Hz, 2H), 7.40-7.27 (m, 3H), 3.80 (s, 3H), 2.30 (s, 3H)

### Step C: methyl 2-(5-amino-2-(furan-2-yl)-8H-pyrazolo[4,3-e][1,2,4]triazolo[1,5-c]pyrimidin-8-yl)-2-phenylpropanoate

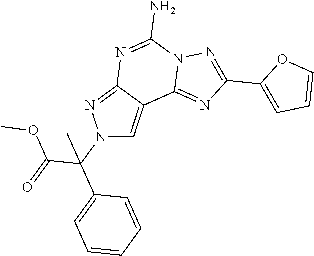 [SMILES: COC(=O)C(C)(c1ccccc1)n1cc2c(nc(N)n3nc(-c4ccco4)nc23)n1]

A mixture of methyl 2-bromo-2-phenylpropanoate (7.57 g, 31.15 mmol), 2-(furan-2-yl)-8H-pyrazolo[4,3-e][1,2,4]triazolo[1,5-c]pyrimidin-5-amine (7.51 g, 31.15 mmol), potassium carbonate (8.60 g, 62.30 mmol) in DMF (30 mL) was stirred at 60° C. overnight. The mixture was extracted with EtOAc (30 mL×3). The combined organic layers were washed with water (10 mL×3) and brine, dried over Na₂SO₄, concentrated to give the product (7.62 g, 60.70%) as white solid. ¹H NMR (400 MHz, DMSO-d6) δ 8.36 (s, 1H), 7.94 (d, J=0.8 Hz, 1H), 7.71 (s, 2H), 7.46-7.40 (m, 3H), 7.30-7.25 (m, 2H), 7.15 (d, J=3.4 Hz, 1H), 6.72 (dd, J=3.3, 1.8 Hz, 1H), 3.77 (s, 3H), 2.30 (s, 3H). MS: M/e 404 (M+1)⁺.

### Step D: 2-(5-amino-2-(furan-2-yl)-8H-pyrazolo[4,3-e][1,2,4]triazolo[1,5-c]pyrimidin-8-yl)-2-phenylpropanoic Acid

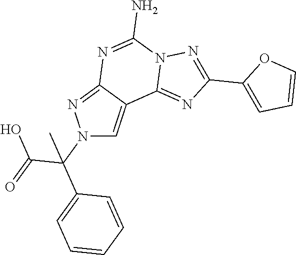 [SMILES: CC(C(=O)O)(c1ccccc1)n1cc2c(nc(N)n3nc(-c4ccco4)nc23)n1]

A mixture of methyl 2-(5-amino-2-(furan-2-yl)-8H-pyrazolo[4,3-e][1,2,4]triazolo[1,5-c]pyrimidin-8-yl)-2-phenylpropanoate (7.62 g, 18.91 mmol), lithium hydroxide (7.563 g, 189.1 mmol) in MeOH (10 mL) and water (5 mL) was stirred at 50° C. overnight. The reaction mixture was acidified with hydrochloric acid, extracted with EtOAc (30 mL×3). The combined organic layers were washed with brine, dried over Na₂SO₄, concentrated and purified by column chromatography (petroleum ether/EtOAc=10:1˜1:2) to give the product (6.20 g, 84.29%) as white solid. MS: M/e 390 (M+1)⁺.

### Step E: 2-(5-amino-2-(furan-2-yl)-8H-pyrazolo[4,3-e][1,2,4]triazolo[1,5-c]pyrimidin-8-yl)-1-(4-(4-(2-methoxyethoxy)phenyl)piperazin-1-yl)-2-phenylpropan-1-one

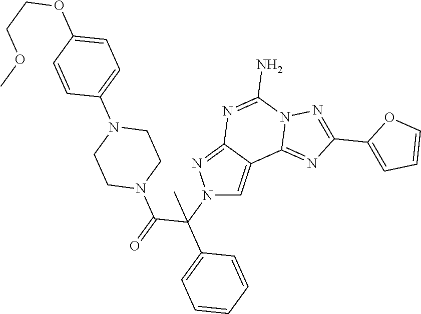 [SMILES: COCCOc1ccc(N2CCN(C(=O)C(C)(c3ccccc3)n3cc4c(nc(N)n5nc(-c6ccco6)nc45)n3)CC2)cc1]

A mixture of 2-(5-amino-2-(furan-2-yl)-8H-pyrazolo[4,3-e][1,2,4]triazolo[1,5-c]pyrimidin-8-yl)-2-phenylpropanoic acid (100 mg, 0.2571 mmol), 1-(4-(2-methoxyethoxy)phenyl)piperazine (91 mg, 0.3856 mmol), HATU (146 mg, 0.3856 mmol), DIPEA (99 mg, 0.7712 mmol) in DMF (3 mL) was stirred at rt overnight. The reaction mixture was poured into H₂O (10 mL) and extracted with EtOAc (15 mL×3). The combined organic layers were washed with brine, dried over Na₂SO₄, concentrated and purified by column chromatography (DCM/MeOH=40:1˜10:1) to give the product (92 mg, 58.96%). ¹H NMR (400 MHz, DMSO-d6) δ 8.69 (s, 1H), 7.94 (s, 1H), 7.71 (s, 2H), 7.39 (dq, J=14.0, 7.0 Hz, 3H), 7.20 (d, J=7.3 Hz, 2H), 7.17 (d, J=3.3 Hz, 1H), 6.86-6.79 (m, 4H), 6.73 (dd, J=3.1, 1.7 Hz, 1H), 4.01-3.97 (m, 2H), 3.83-3.69 (m, 4H), 3.62-3.58 (m, 2H), 3.28 (s, 3H), 3.01 (s, 4H), 2.29 (s, 3H). MS: M/e 608 (M+1)⁺.

Compound C25 was separated into two enantiomeric stereoisomers, Compound C25a (earlier peak), and Compound C25b (later peak) by chiral prep-HPLC. The chiral separation conditions are shown below.

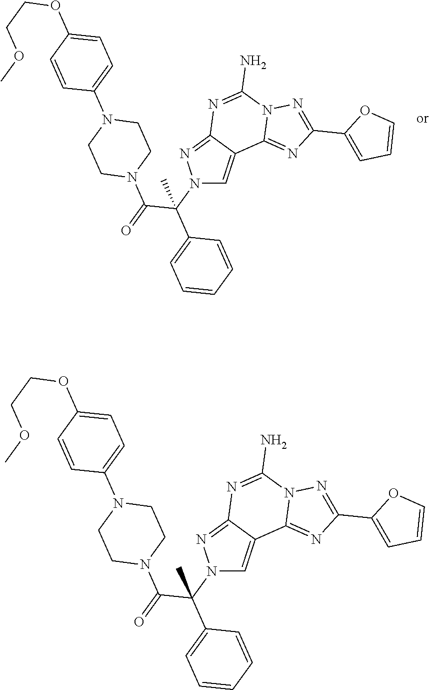 [SMILES: COCCOc1ccc(N2CCN(C(=O)[C@](C)(c3ccccc3)n3cc4c(nc(N)n5nc(-c6ccco6)nc45)n3)CC2)cc1 ; COCCOc1ccc(N2CCN(C(=O)[C@@](C)(c3ccccc3)n3cc4c(nc(N)n5nc(-c6ccco6)nc45)n3)CC2)cc1]

| Column | CHIRAL ART Cellulose-SB |
|---|---|
| Column size | 2 cm × 25 cm, 5 um |
| Injection | 1 mL |
| Mobile phase | MeOH:DCM = 60:40 |
| Flow rate | 20 ml/min |
| Wavelength | UV 220 nm |
| Temperature | 25° C. |
| Sample solution | 29.4 mg/ml in MeOH:DCM = 3:1 |

### Compound D2: 2-(5-amino-2-(furan-2-yl)-8H-pyrazolo[4,3-e][1,2,4]triazolo[1,5-c]pyrimidin-8-yl)-1-(4-(4-(2-methoxyethoxy)phenyl)piperazin-1-yl)ethan-1-one and Compound D1: 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)ethan-1-one

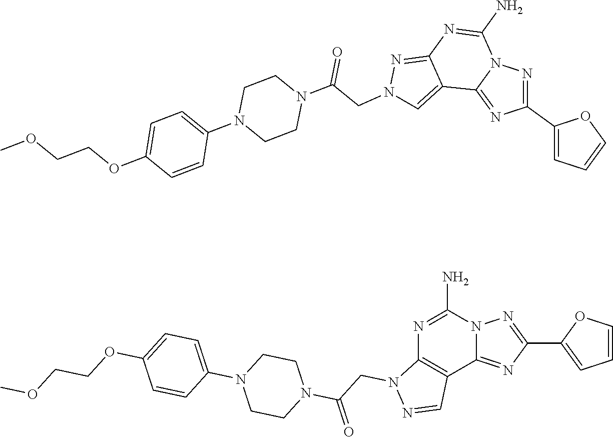 [SMILES: COCCOc1ccc(N2CCN(C(=O)Cn3cc4c(nc(N)n5nc(-c6ccco6)nc45)n3)CC2)cc1 ; COCCOc1ccc(N2CCN(C(=O)Cn3ncc4c3nc(N)n3nc(-c5ccco5)nc43)CC2)cc1]

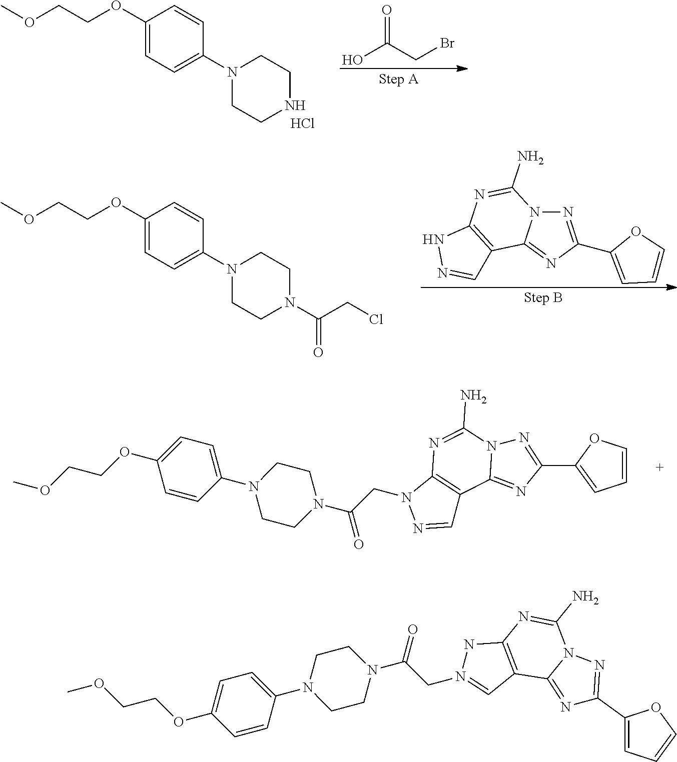

### Step A: 2-chloro-1-(4-(4-(2-methoxyethoxy)phenyl)piperazin-1-yl)ethan-1-one (Obtained)

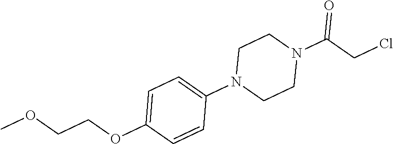 [SMILES: COCCOc1ccc(N2CCN(C(=O)CCl)CC2)cc1]

To a mixture of 1-(4-(2-methoxyethoxy)phenyl)piperazine hydrochloride (135 mg, 0.5 mmol), 2-bromoacetic acid (70 mg, 0.5 mmol) and HATU (190 mg, 0.5 mmol) in DMF (4 mL) was added TEA (101 mg, 1 mmol). The reaction was stirred at rt overnight. The mixture was quenched with water (30 mL), extracted with DCM (30 mL×3), washed with brine, dried over Na₂SO₄, filtered and concentrated. The residue was purified by column chromatography (eluting with EA:PE=1: 1) to give the product (125 mg, 80%) as a solid. The product was 2-chloro-1-(4-(4-(2-methoxyethoxy)phenyl)piperazin-1-yl)ethan-1-one. MS: M/e 313 (M+1)⁺.

### Step B: 2-(5-amino-2-(furan-2-yl)-8H-pyrazolo[4,3-e][1,2,4]triazolo[1,5-c]pyrimidin-8-yl)-1-(4-(4-(2-methoxyethoxy)phenyl)piperazin-1-yl)ethan-1-one and 2-(5-amino-2-(furan-2-yl)-7H-pyrazolo[4,3-e][1,2,4]triazolo[1,5-c]pyrimidin-7-yl)-1-(4-(4-(2-methoxyethoxy)phenyl)piperazin-1-yl)ethan-1-one

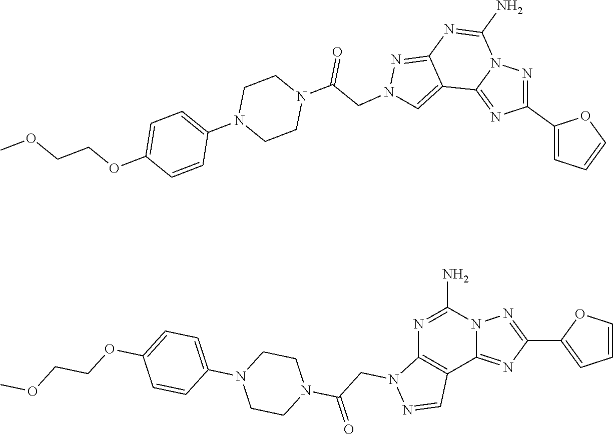 [SMILES: COCCOc1ccc(N2CCN(C(=O)Cn3cc4c(nc(N)n5nc(-c6ccco6)nc45)n3)CC2)cc1 ; COCCOc1ccc(N2CCN(C(=O)Cn3ncc4c3nc(N)n3nc(-c5ccco5)nc43)CC2)cc1]

To a mixture of the product of the step A (125 mg, 0.4 mmol) and 2-(furan-2-yl)-7H-pyrazolo[4,3-e][1,2,4]triazolo[1,5-c]pyrimidin-5-amine (80 mg, 0.33 mmol) in DMF (2 mL) was added K₂CO₃ (68 mg, 0.49 mmol). The reaction was stirred at rt for 4 days. The reaction was quenched with water (30 mL), extracted with DCM (50 mL×4), washed with brine, dried over Na₂SO₄, filtered and concentrated. The residue was purified by prep-HPLC to give the two isomers (13 mg+5 mg). Compound D2: ¹H NMR (400 MHz, DMSO-d6) δ 8.53 (s, 1H), 7.94 (s, 1H), 7.67 (br.s, 2H), 7.21 (d, J=3.6 Hz, 1H), 6.97 (d, J=8.8 Hz, 2H), 6.87 (d, J=8.8 Hz, 2H), 6.76-6.72 (m, 1H), 5.40 (s, 2H), 4.03 (t, J=4.8 Hz, 2H), 3.80-3.67 (m, 4H), 3.63 (t, J=4.8 Hz, 2H), 3.30 (s, 3H), 3.17-3.11 (m, 2H), 3.09-3.02 (m, 2H); MS: M/e 518 (M+1)⁺.

Compound D1: ¹H NMR (400 MHz, DMSO-d6) δ 8.19 (s, 1H), 8.10 (br.s, 2H), 7.95 (s, 1H), 7.25 (d, J=3.2 Hz, 1H), 6.95 (d, J=8.8 Hz, 2H), 6.86 (d, J=8.8 Hz, 2H), 6.76-6.72 (m, 1H), 5.30 (s, 2H), 4.02 (t, J=4.0 Hz, 2H), 3.80-3.70 (m, 4H), 3.63 (t, J=4.0 Hz, 2H), 3.28 (s, 3H), 3.16-3.108 (m, 2H), 3.05-2.96 (m, 2H); MS: M/e 518 (M+1)⁺.

### Cell Culture and Transfection

HEK293 cells were maintained in Dulbecco's modified Eagle's medium (DMEM, Gibco) supplemented with 10% fetal bovine serum (FBS, Thermo Scientific), 100 units/mL penicillin (Gibco), and 0.1 mg/mL streptomycin (Gibco) in a humidified 37° C. environment with 5% C02. Plasmid encoding wild-type human A2A receptor (A2AR) (in pcDNA3.1) was synthesized by Genscript (Nanjing, China). Transfection of the plasmids was performed in 6-well plates with 4×105 cells using Lipofectamin 2000 (ThermoFisher Scientific) according to the instruction of the manufacturer. Cell clones that stably express A2AR were established and maintained in the same complete medium as the HEK293 cells in addition of G-418 (Gibico). Expression level of A2AR in each single cell clone was determined using immunoblotting and FACS method. HEK293-A2AR stable cells were then transfected with pGL4.29[luc2P/CRE/Hygro] (Promega) luciferase reporter plasmid for establishing the HEK293-A2AR-luc2p/CRE/Hygro stable cell line.

### Luciferase Reporter Assay

HEK293-A2AR-luc2p/CRE/Hygro cells were seeded at a density of 5,000 cells/well in DMEM with 1% FBS and 1 U/mL adenosine deaminase (ADA) (Sigma). After 18 h, the cells were treated with 3 nM CGS21680 plus a series dilution of A2AR antagonist, the compounds disclosed herein at the concentration of 0.1˜10000 nM, prepared in DMEM with 1% FBS. After 5 h incubation, the luciferase activity in cells were measured using the Bright-Glo Luciferase Assay System (Promega) according to manufacturer's instructions. The luminescence signal was measured using a PHERAstar FS plate reader (BMG Labtech). Luminescence intensity from 10 μM preladenant treatment was set as 0%. Maximal luminescence intensity was determined in the presence of 3 nM CGS21680 and was set as 100%. The IC50 value was calculated from a dose dependent inhibition curve across the range of compound concentrations.

### Adenosine Receptor Binding Assay

Binding affinity of test compounds to four human adenosine receptors, A1, A2A, A2B and A3 was determined in radioligand competitive binding assay (conducted by Cerep, France) using following protocols. For A1 receptor (A1R), membrane homogenates from CHO cells transfected with A1R were incubated for 60 min at 22° C. with 1 nM [3H]DPCPX in the absence or presence of the test compound in a buffer containing 50 mM Tris-HCl (pH 7.4), 5 mM MgCl2, 1 mM EDTA/Tris and 2 UI/mL ADA. For A2AR, membrane homogenates from HEK293 cells transfected with A2AR were incubated for 120 min at 22° C. with 6 nM [3H]CGS21680 in the absence or presence of the test compound in a buffer containing 50 mM Tris-HCl (pH 7.4), 10 mM MgCl2, and 2 UI/mL ADA. For A2B receptor (A2BR), membrane homogenates from HEK293 cells transfected with A2BR were incubated for 60 min at 22° C. with 5 nM [3H]CPX in the absence or presence of the test compound in a buffer containing 10 mM Hepes/Tris (pH 7), 1 mM MgCl2, and 1 mM EDTA. For A3 receptor (A3R), membrane homogenates from HEK293 cells transfected with A3R were incubated for 120 min at 22° C. with 0.15 nM [125I]AB-MECA in the absence or presence of the test compound in a buffer containing 50 mM Tris-HCl (pH 7.4), 5 mM MgCl2, 1 mM EDTA and 2 UI/mL ADA. Nonspecific binding was determined in the presence of unlabeled 1 μM DPCPX, 10 μM NECA, 100 μM NECA, and 1 μM IB-MECA in A1R, A2AR, A2BR, A3R binding assays, respectively. Following incubation, the samples are rapidly filtered and washed with ice-cold 50 mM Tris-HCl. Then filters are dried and counted for radioactivity in a scintillation counter (Topcount, Packard) using a scintillation cocktail (Microscint 0, Packard). Duplicate experiments were performed for each assay. The results are expressed as a percent inhibition of control radioligand specific binding.

### Mouse BBB Assay

Mice were acclimated for 1 week prior to use at 18-30 g body weight. Fasted mice were dosed orally at 10 mg/kg dose. At 1, 2 and 4 hours postdose, plasma samples from cardiac blood collected in tubes containing K₂EDTA as the anticoagulant, and excised cerebral hemispheres were immediately frozen and stored at −80° C. until bioanalysis. Total concentrations of the compound were determined by LC-MS/MS. Brain homogenate concentrations were converted to brain concentrations for the calculations of brain-to-plasma ratios.

TABLE 1
Results of Luciferase reporter assay

| Com- | IC50 | Com- | IC50 |
|---|---|---|---|
| pound No. | (nM) | pound No. | (nM) |
| A1 | 42.9 | A2 | 96.9 |
| A3 | 362.2 | A4 | 43.5 |
| A5 | 9.1 | A6 | 9.5 |
| A7 | 23.1 | A8 | 41.5 |
| A9 | 59.1 | A10 | 6.3 |
| A11 | 6.9 | A12 | 113.3 |
| A13 | 2.9 | A14 | 12.7 |
| A15 | 8.4 | A16 | 1.3 |
| A17 | 1.1 | A18 | 30.6 |
| A19 | 6.5 | A20 | 4.3 |
| A21 | 21.6 | A22 | 1.5 |
| A23 | 1.4 | A24 | 4.4 |
| A25 | 4.5 | A26 | 43.8 |
| A27 | 39.4 | A28 | 7.6 |
| A29 | 20.5 | A30 | 22.0 |
| A31 | 0.6 | A32 | 7.2 |
| A33 | 13.3 | A34 | 75.7 |
| A35 | 5.8 | A36 | 7.7 |
| A37 | 2.5 | A38 | 0.6 |
| A39 | 5.3 | A40 | 23.7 |
| A41 | 5.8 | A42 | 5.1 |
| A43 | 0.9 | A44 | 2.0 |
| A45 | 6.0 | A46 | 2.9 |
| A47 | 2.8 | A48 | 1.6 |
| A49 | 3.2 | A50 | 1.5 |
| A51 | 4.8 | A52 | 8.7 |
| A53 | 5.0 | A54 | 4.9 |
| A55 | 2.3 | A56 | 1.7 |
| A57 | 5.3 | A58 | >10000 |
| A59 | 18.4 | A60 | >10000 |
| A61 | 0.5 | A62 | 1.3 |
| A63 | 6.2 | A64 | 4.8 |
| A65 | 6.4 | A66 | 6.5 |
| A67 | 8.9 | A68 | 4.4 |
| A69 | 3.4 | A70 | 5.0 |
| A71 | 7.5 | A72 | 103.1 |
| A73 | 15.6 | A74 | 5.1 |
| A75 | 6.4 | A76 | 7.3 |
| A77 | 4.2 | A78 | 6.2 |
| A79 | 7.1 | A80 | 4.3 |
| A81 | 4.7 | A82 | 7.1 |
| A83 | 3.7 | A84 | 28.3 |
| A85 | 11.6 | A86 | 6.8 |
| A87 | 2.0 | A88 | 7.0 |
| A89 | 13.8 | A90 | 14.1 |
| A91 | 3.3 | A92 | 2.7 |
| A93 | 0.6 | A94 | 3.8 |
| A95 | 31.1 | A96 | 13.7 |
| A97 | 335.4 | A98 | 7.2 |
| A99 | 28.6 | A100 | 25.0 |
| A101 | 3.9 | A102 | 9.2 |
| A103 | 1.7 | A104 | 5.5 |
| A105 | 10.3 | A106 | 8.5 |
| A107 | 4.8 | A108 | 3.5 |
| B1 | 15.2 | B2 | 2.9 |
| B3 | 1.9 | B4 | 2.9 |
| B5 | 3.0 | B6 | 7.0 |
| B7 | 2.0 | B8 | 4.0 |
| B9 | 3.4 | B10 | 0.8 |
| B11 | 2.2 | B12 | 2.5 |
| B13 | 2.8 | B14 | 5.7 |
| B15 | 2.2 | B16 | 5.6 |
| B17 | 1.3 | B18 | 111.3 |
| B19 | 111.6 | B20 | 20.0 |
| B21 | 96.7 | B22 | 16.0 |
| B23 | 24.9 | B24 | 12.0 |
| B25 | 23.7 | B26 | 15.4 |
| B27 | 30.3 | B28 | 33.5 |
| B29 | 76.5 | B30 | 97.3 |
| B31 | 46.0 | B32 | 111.2 |
| B33 | 16.5 | B34 | 14.0 |
| C1 | 28.4 | C2 | 648.5 |
| C3 | 1874.0 | C4 | 140.5 |
| C5 | 132.0 | C6 | 3178.0 |
| C7 | 185.9 | C8 | 242.5 |
| C9 | 39.8 | C10 | 125.9 |
| C11 | 118.2 | C12 | 336.5 |
| C13 | 12.7 | C14 | 9.5 |
| C15 | 17.0 | C16 | 4.9 |
| C17 | 88.4 | C18 | 90.2 |
| C19 | 46.3 | C20 | 2065.0 |
| C21 | 100.2 | C22 | 12.8 |
| C23 | 69.6 | C24 | 35.5 |
| C25 | 21.7 | | |
| D1 | 27.0 | D2 | 66.8 |
| A1a | 223.2 | A1b | 40.0 |
| A5a | 2.2 | A5b | 50.0 |
| A6a | 5.0 | A6b | 134.6 |
| A10a | 2.7 | A10b | 143.1 |
| A11a | 4.6 | A11b | 121.1 |
| A13a | 26.9 | A13b | 0.8 |
| A16a | 0.3 | A16b | 96.4 |
| A17a | 0.9 | A17b | 160.3 |
| A22a | 245.6 | A22b | 0.6 |
| A24a | 1.0 | A24b | 78.8 |
| A31a | 8.1 | A31b | 0.1 |
| A39a | 127.0 | A39b | 3.6 |
| A43a | 16.1 | A43b | 0.4 |
| A44a | 39.2 | A44b | 0.5 |
| A46a | 67.4 | A46b | 0.9 |
| A47a | 17.3 | A47b | 0.2 |
| A50a | 88.2 | A50b | 1.0 |
| A63a | 8.0 | A63b | 3.3 |
| A64a | 17.5 | A64b | 2.2 |
| A65a | 21.4 | A65b | 3.3 |
| A66a | 16.8 | A66b | 3.3 |
| A70a | 10.1 | A70b | 3.4 |
| A71a | 10.9 | A71b | 4.8 |
| A76a | 19.1 | A76b | 3.9 |
| A79a | 21.4 | A79b | 6.8 |
| A80a | 7.4 | A80b | 2.8 |
| A81a | 7.9 | A81b | 2.6 |
| A82a | 12.2 | A82b | 4.9 |
| B3a | 21.6 | B3b | 0.5 |
| B7a | 29.1 | B7b | 0.5 |
| C1a | 57.1 | C1b | 12.7 |
| C5a | 55.0 | C5b | 209.4 |
| C16a | 4.1 | C16b | 167.9 |
| C25a | 79.8 | C25b | 21.2 |
| D1 | 27 | D2 | 66.8 |

TABLE 2
Results of binding assay

| Compound No. | A1 | A2A |
|---|---|---|
| A5a | Ki = 160 nM | Ki = 1.7 nM |
| A5b | Ki = 310 nM | Ki = 6 nM |
| A6a | Ki = 81 nM | Ki = 1.4 nM |
| A10a | Ki = 120 nM | Ki = 2.3 nM |
| A10b | Ki = 260 nM | Ki = 5.6 nM |
| A13a | Ki = 190 nM | Ki = 6.2 nM |
| A13b | Ki = 42 nM | Ki = 0.92 nM |
| A16a | Ki = 57 nM | Ki = 1.5 nM |
| A16b | Ki = 770 nM | Ki = 15 nM |
| A22b | Ki = 18 nM | Ki = 1.7 nM |
| A24a | Ki = 120 nM | Ki = 1.4 nM |
| A24b | Ki = 1200 nM | Ki = 8.5 nM |
| A39b | Ki = 79 nM | Ki = 2 nM |
| A43b | Ki = 54 nM | Ki = 1.2 nM |
| A63a | Ki = 1130 nM | Ki = 3.1 nM |
| A63b | Ki = 153 nM | Ki = 1.5 nM |
| B3b | Ki = 150 nM | Ki = 0.89 nM |
| C1a | Ki < 0.5 nM | Ki = 6.2 nM |
| C1b | Ki = 10 nM | Ki = 2.5 nM |
| D1 | Ki = 52 nM | Ki = 1.8 nM |
| D2 | Ki = 0.81 nM | Ki = 2.8 nM |
| Preladenant | Ki = 337 nM | Ki = 1.3 nM |

TABLE 3
Results of Mouse BBB assay at 1 h

| | Plasma | Brain |
|---|---|---|
| Compound No. | (ng · mL⁻¹) | (ng · g⁻¹) |
| A5a | 116 | 5.1 |
| A6a | 173 | 4.2 |
| A10a | 471 | 20 |
| A13b | 2443 | 54 |
| Preladenant | 197 | 258 |

It is to be understood that, if any prior art publication is referred to herein; such reference does not constitute an admission that the publication forms a part of the common general knowledge in the art in any country.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and Examples should not be construed as limiting the scope of the invention.

What is claimed is:

1. A compound of formula (I) 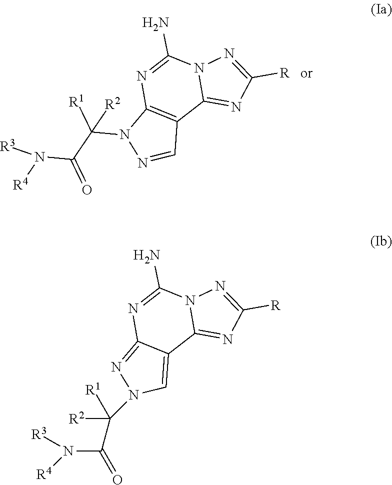
or a stereoisomer thereof, or a pharmaceutically acceptable salt thereof,
wherein:
R is a aryl group or a 5 or 6-membered heteroaryl group containing 1 or 2 heteroatoms independently selected from nitrogen, oxygen or optionally oxidized sulfur as ring member(s), and said ring is optionally substituted with at least one substituent R⁸;
R¹ and R², which may be the same or different, are each independently selected from hydrogen, —C₁₋₆alkyl, —C₂₋₆alkenyl, —C₂₋₆alkynyl, —C₃₋₈cycloalkyl, heterocyclyl, aryl, or heteroaryl, wherein said C₁₋₆alkyl, —C₂₋₆alkenyl, —C₂₋₆alkynyl, —C₃₋₈cycloalkyl, heterocyclyl, aryl, or heteroaryl are each independently optionally substituted with at least one substituent R⁸, provided that at least one of R¹ and R² is not hydrogen; or
R¹ and R², together with the carbon atom to which they are attached, form a 3- to 12-membered saturated, partially or fully unsaturated ring comprising 0, 1 or 2 heteroatoms independently selected from nitrogen, oxygen or optionally oxidized sulfur as ring member(s), and said ring is optionally substituted with at least one substituent R⁸;
R³ and R⁴, together with the nitrogen atom to they are attached, form a 3- to 12-membered ring, said ring comprising 0, 1 or 2 additional heteroatoms independently selected from nitrogen, oxygen or optionally oxidized sulfur as ring member(s), said ring is optionally substituted with one or two or three substituents R⁵;
R⁵ is independently hydrogen, halogen, —C₁₋₆alkyl, —C₂₋₆alkenyl, —C₂₋₆alkynyl, C₃₋₈cycloalkyl, heterocyclyl, aryl, heteroaryl, —CN, —NO₂, oxo, —OR^{5a}, —SO₂R^{5a}, —COR^{5a}, —CO₂R^{5a}, —CONR^{5a}R^{5b}, —C(═NR^{5a})NR^{5b}R^{5c}, —NR^{5a}R^{5b}, —NR^{5a}COR^{5b}, —NR^{5a}CONR^{5b}R^{5c}, —NR^{5a}CO₂R^{5b}, —NR^{5a}SONR^{5b}R^{5c}, —NR^{5a}SO₂NR^{5b}R^{5c}, or —NR^{5a}SO₂R^{5b}, wherein, as R⁵, each of said —C₁₋₆alkyl, —C₂₋₆alkenyl, —C₂₋₆alkynyl, C₃₋₈cycloalkyl, heterocyclyl, aryl, or heteroaryl is independently and optionally substituted with one or two or three substituents R⁶;
R^{5a}, R^{5b}, and R^{5c}, which may be the same or different, are each independently hydrogen, —C₁₋₆alkyl, —C₂₋₆alkenyl, —C₂₋₆alkynyl, cycloalkyl, heterocyclyl, aryl, or heteroaryl, wherein said —C₁₋₆alkyl, —C₂₋₆alkenyl, —C₂₋₆alkynyl, cycloalkyl, heterocyclyl, aryl, or heteroaryl are each independently optionally substituted with one or two substituent R^{5d};
R^{5d} is independently hydrogen, halogen, cyano, —C₁₋₆alkyl, —C₂₋₆alkenyl, —C₂₋₆alkynyl, haloC₁₋₆alkyl, haloC₂₋₆alkenyl, haloC₂₋₆alkynyl, —C₁₋₆alkoxy, C₁₋₆alkoxy-C₁₋₆alkoxy-, C₂₋₆alkenyloxy-, C₂₋₆alkynyloxyl-, haloC₁₋₆alkoxy-, haloC₂₋₆alkenyloxy-, haloC₂₋₆alkynyloxy-, C₃₋₈cycloalkyloxy-, cycloalkyl, heterocyclyl, heterocyclyloxy-, aryl, aryloxy-, heteroaryl or heteroaryloxy-;
R⁶ is independently hydrogen, halogen, —C₁₋₆alkyl, —C₂₋₆alkenyl, —C₂₋₆alkynyl, C₃₋₈cycloalkyl, heterocyclyl, aryl, heteroaryl, —CN, —NO₂, oxo, —OR^{6a}, —SO₂R^{6a}, —COR^{6a}, —CO₂R^{6a}, —CONR^{6a}R^{6b}, —C(═NR^{6a})NR^{6b}R^{6c}, —NR^{6a}R^{6b}, —NR^{6a}COR^{6b}, —NR^{6a}CONR^{6b}R^{6c}, —NR^{6a}CO₂R^{6b}, —NR^{6a}SONR^{6b}R^{6c}, —NR^{6a}SO₂NR^{6b}R^{6c}, or NR^{6a}SO₂R^{6b};
R^{6a}, R^{6b}, and R^{6c}, which may be the same or different, are each independently hydrogen, halogen, —C₁₋₆alkyl, —C₂₋₆alkenyl, —C₂₋₆alkynyl, —C₃₋₈cycloalkyl, heterocyclyl, aryl, or heteroaryl, wherein said C₁₋₆alkyl, —C₂₋₆alkenyl, —C₂₋₆alkynyl, cycloalkyl, heterocyclyl, aryl, or heteroaryl are each independently optionally substituted with one or two or three substituent R⁷; or
(R^{6a} and R^{6b}), and/or (R^{6b} and R^{6c}), and/or (R^{6c} and R^{6a}), together with the atom(s) to which they are attached, form a 3- to 12-membered saturated, partially or fully unsaturated ring comprising 0, 1 or 2 additional heteroatoms independently selected from nitrogen, oxygen or optionally oxidized sulfur as ring member(s), and said ring is optionally substituted with at least one substituent R⁸;
R⁷ is independently hydrogen, halogen, —C₁₋₆alkyl, —C₂₋₆alkenyl, —C₂₋₆alkynyl, C₃₋₈cycloalkyl, heterocyclyl, aryl, heteroaryl, —CN, —NO₂, oxo, —OR^{7a}, —SO₂R^{7a}, —COR^{7a}, —CO₂R^{7a}, —CONR^{7a}R^{7b}, —C(═NR^{7a})NR^{7b}R^{7c}, —NR^{7a}R^{7b}, —NR^{7a}COR^{7b}, —NR^{7a}CONR^{7b}R^{7c}, —NR^{7a}CO₂R^{7b}, —NR^{7a}SONR^{7b}R^{7c}, —NR^{7a}SO₂NR^{7b}R^{7c}, or —NR^{7a}SO₂R^{7b}, wherein said —C₁₋₆alkyl, —C₂₋₆alkenyl, —C₂₋₆alkynyl, C₃₋₈cycloalkyl, heterocyclyl, aryl, or heteroaryl are each independently optionally substituted with one or two substituents selected from halogen, hydroxy, —C₁₋₆alkyl, —C₁₋₆alkoxy, oxo, cyano, and amino;
R^{7a}, R^{7b}, and R^{7c} each is independently hydrogen, —C₁₋₆alkyl, C₁₋₆alkoxy-C₁₋₆alkyl-, —C₂₋₆alkenyl, —C₂₋₆alkynyl, C₃₋₈cycloalkyl, heterocyclyl, aryl, or heteroaryl; and
R₈ is independently hydrogen, halogen, cyano, oxo, amino, —C₁₋₆alkyl, —C₂₋₆alkenyl, —C₂₋₆alkynyl, haloC₁₋₆alkyl, haloC₂₋₆alkenyl, haloC₂₋₆alkynyl, —C₁₋₆alkoxy, C₃₋₈cycloalkyloxy, cycloalkyl, heterocyclyl, aryl, or heteroaryl.

2. The compound according to claim 1, wherein R is a C-linked 5 or 6-membered heteroaryl group containing 1 or 2 heteroatoms independently selected from nitrogen, oxygen or optionally oxidized sulfur as ring member(s).

3. The compound according to claim 2, wherein R is furanyl, pyrazinyl or thiazolyl; preferably, furan-2-yl, 3-methylpyrazin-2-yl or thiazol-2-yl.

4. The compound according to claim 1, wherein R¹ is hydrogen or C₁₋₆alkyl, preferably hydrogen, methyl, ethyl; more preferably hydrogen; and R² is C₁₋₆alkyl (preferably methyl, isopropyl, ethyl, propyl, butyl, or isobutyl) optionally substituted with phenyl or —C₁₋₆alkoxy (preferably methoxy); aryl (i.e., phenyl or naphthyl) optionally substituted with halogen or C₁₋₆alkoxy; —C₃₋₈cycloalkyl (preferably cyclopropyl), or heterocyclyl.

5. The compound according to any one of claims 1-4, wherein R³ and R⁴, together with the nitrogen atom to they are attached, form a 3-, 4-, 5-, 6-, 7-, 8-, or 9-membered monocyclic ring comprising 0, 1 or 2 additional heteroatoms independently selected from nitrogen, oxygen or optionally oxidized sulfur as ring member(s), said ring is optionally substituted with one or two or three substituents R⁵.

6. The compound according to claim 5, wherein R³ and R⁴, together with the nitrogen atom to they are attached, form a 3-, 4-, 5-, 6-, 7-, 8-, or 9-membered monocyclic ring comprising 0 additional heteroatom independently selected from nitrogen, oxygen or optionally oxidized sulfur as ring member(s), said ring is optionally substituted with one or two or three substituents R⁵.

7. The compound according to claim 5, wherein R³ and R⁴, together with the nitrogen atom to they are attached, form a 3-, 4-, 5-, 6-, 7-, 8-, or 9-membered monocyclic ring comprising 1 additional heteroatom independently selected from nitrogen, oxygen or optionally oxidized sulfur as ring member(s), said ring is optionally substituted with one or two or three substituents R⁵.

8. The compound according to claim 7, wherein R³ and R⁴, together with the nitrogen atom to they are attached, form a 3-, 4-, 5-, 6-, 7-, 8-, or 9-membered monocyclic ring comprising one additional nitrogen heteroatom as ring member, said ring is optionally substituted with one or two or three substituents R⁵.

9. The compound according to any one of claims 5-9, wherein said ring is saturated.

10. The compound according to claim 5, wherein R³ and R⁴, together with the nitrogen atom to they are attached, form an azetidinyl 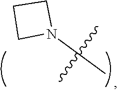
pyrrolidinyl 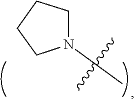
or piperidinyl 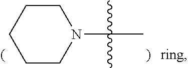 [SMILES: *N1CCCCC1]
each of which is optionally substituted with R⁵ as defined with Formula (Ia) or (Ib).

11. The compound according to claim 5, wherein R³ and R⁴, together with the nitrogen atom to they are attached, form a piperazinyl ring optionally substituted with R⁵ as defined with Formula (Ia) or (Ib)

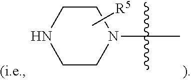

12. The compound according to any one of claims 1-4, wherein R³ and R⁴, together with the nitrogen atom to they are attached, form a 7-, 8-, 9-, 10-, 11-, or 12-membered bicyclic ring comprising 0, 1 or 2 additional heteroatoms independently selected from nitrogen, oxygen or optionally oxidized sulfur as ring member(s), said ring is optionally substituted with one or two or three substituents R⁵.

13. The compound according to claim 12, wherein R³ and R⁴, together with the nitrogen atom to they are attached, form a 7-, 8-, 9-, 10-, 11-, or 12-membered bicyclic fused ring comprising 0, 1 or 2 additional heteroatoms independently selected from nitrogen, oxygen or optionally oxidized sulfur as ring member(s), said ring is optionally substituted with one or two or three substituents R⁵.

14. The compound according to claim 12, wherein R³ and R⁴, together with the nitrogen atom to they are attached, form a 7-, 8-, 9-, 10-, 11-, or 12-membered bicyclic fused ring comprising 0 or 1 additional heteroatom independently selected from nitrogen, oxygen or optionally oxidized sulfur as ring member(s), said ring is optionally substituted with one or two or three substituents R⁵.

15. The compound according to claim 12, wherein R³ and R⁴, together with the nitrogen atom to they are attached, form a 10-membered bicyclic fused ring comprising 0 or 1 additional nitrogen heteroatom as ring member, said ring is optionally substituted with one or two or three substituents R⁵.

16. The compound according to claim 15, wherein R³ and R⁴ together with the nitrogen atom to they are attached, form a 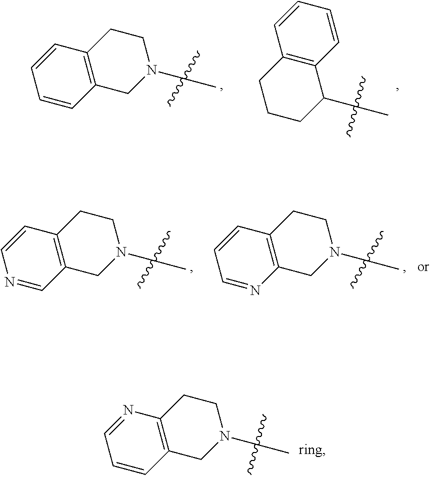 [SMILES: *N1CCc2ccccc2C1 ; *C1CCCc2ccccc21 ; *N1CCc2ccncc2C1 ; *N1CCc2cccnc2C1 ; *N1CCc2ncccc2C1]
each of said ring is optionally substituted with one or two or three substituents R⁵.

17. The compound according to claim 12, wherein R³ and R⁴, together with the nitrogen atom to they are attached, form a 7-, 8-, 9-, 10-, 11-, or 12-membered bicyclic spiro ring comprising 0, 1 or 2 additional heteroatoms independently selected from nitrogen, oxygen or optionally oxidized sulfur as ring member(s), said ring is optionally substituted with one or two or three substituents R⁵.

18. The compound according to claim 17, wherein R³ and R⁴, together with the nitrogen atom to they are attached, form azaspiro[3.3]heptane, azaspiro[3.5]nonane, azaspiro[3.4]octane, azaspiro[5.5]undecane, or azaspiro[4.5]decane, each of which comprises 0 or 1 additional nitrogen or oxygen atom as ring member, and said ring is optionally substituted with one or two or three substituents R⁵.

19. The compound according to claim 18, wherein R³ and R⁴, together with the nitrogen atom to they are attached, form a 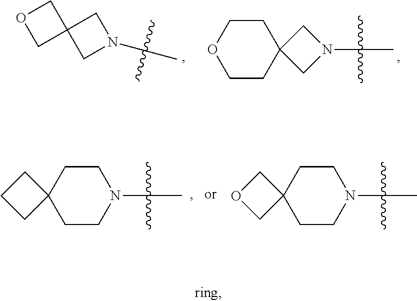 [SMILES: *N1CC2(COC2)C1 ; *N1CC2(CCOCC2)C1 ; *N1CCC2(CCC2)CC1 ; *N1CCC2(CC1)COC2]
and said ring is optionally substituted with one or two or three substituents R⁵.

20. The compound according to any one of claims 5-19, wherein said ring is substituted with one R⁵.

21. The compound according to any one of claims 5-19, wherein said ring is substituted with two R⁵.

22. The compound according to claim 21, wherein said ring is substituted with one C₁₋₆alkyl (preferably methyl) and further substituted with one R⁵.

23. The compound according to any one of claims 20-22, wherein R⁵ is halogen, —C₁₋₆alkyl, aryl, —OR^{5a}, or —CO₂R^{5a}, wherein R^{5a} is as defined with formula (Ia) or (Ib).

24. The compound according to claim 23, wherein R⁵ is —CO₂R^{5a}, wherein R^{5a} is —C₁₋₆alkyl, preferably methyl.

25. The compound according to claim 23, wherein R⁵ is —OR^{5a}, wherein R^{5a} is —C₁₋₆alkyl, optionally substituted with one R^{5d}, wherein R^{5d} is hydrogen, halogen (preferably fluoro), or —C₁₋₆alkoxy.

26. The compound according to claim 23, wherein R⁵ is —OR^{5a}, wherein R^{5a} is trifluoromethoxy, methoxy, methoxyethoxy, or hydroxy.

27. The compound according to claim 23, wherein R⁵ is phenyl, optionally substituted with one or two or three substituents R⁶, wherein R⁶ is defined as with Formula (Ia) or (Ib).

28. The compound according to claim 27, wherein R⁵ is phenyl, optionally substituted with one or two or three substituents R⁶, wherein R⁶ is independently halogen (preferably fluoro), —OR^{6a}, or NR^{6a}R^{6b}C(O)—, wherein R^{6a} and R^{6b} are defined as with Formula (Ia) or (Ib).

29. The compound according to claim 28, wherein R⁵ is phenyl, optionally substituted with one substituent R⁶, wherein
R⁶ is NR^{6a}R^{6b}C(O)—, wherein
R^{6a} and R^{6b} are each independently hydrogen, —C₁₋₆alkyl, or —C₃₋₈cycloalkyl, said —C₁₋₆alkyl and —C₃₋₈cycloalkyl are each optionally substituted with one R⁷, wherein
R⁷ is heterocyclyl (preferably 3- to 8-membered heterocyclic comprising one or two heteroatoms independently selected from nitrogen, oxygen or optionally oxidized sulfur as ring member(s), optionally substituted with hydroxy, —C₁₋₆alkyl, or —C₁₋₆alkoxy.

30. The compound according to claim 28, wherein R⁵ is phenyl, optionally substituted with one substituent R⁶, wherein
R⁶ is NR^{6a}R^{6b}C(O)—, wherein
R^{6a} and R^{6b} are each independently hydrogen, —C₁₋₆alkyl, or —C₃₋₈cycloalkyl, said —C₁₋₆alkyl is optionally substituted with one R⁷, wherein
R⁷ is 3- to 8-membered saturated monocyclic heterocyclic comprising one heteroatom selected from nitrogen or oxygen as ring member (preferably oxetanyl), optionally substituted with hydroxy.

31. The compound according to claim 28, wherein R⁵ is phenyl, optionally substituted with one substituent R⁶, wherein
R⁶ is NR^{6a}R^{6b}C(O)—, wherein
R^{6a} and R^{6b}, together with the nitrogen atom to which they are attached, form a 3- to 12-membered saturated ring comprising 0, 1 or 2 additional heteroatoms independently selected from nitrogen, oxygen or optionally oxidized sulfur as ring member(s).

32. The compound according to claim 31, wherein R^{6a} and R^{6b}, together with the nitrogen atom to which they are attached, form a 3- to 8-membered saturated monocyclic ring comprising 0 or 1 additional heteroatom independently selected from nitrogen or oxygen as ring member.

33. The compound according to claim 32, wherein R^{6a} and R^{6b}, together with the nitrogen atom to which they are attached, form a 6-membered saturated monocyclic ring comprising 0 or 1 additional nitrogen heteroatom as ring member.

34. The compound according to claim 31, wherein R^{6a} and R^{6b}, together with the nitrogen atom to which they are attached, form a piperidinyl ring.

35. The compound according to claim 28, wherein R⁵ is phenyl, optionally substituted with one or two halogen and further optionally substituted with one substituent R⁶, wherein
R⁶ is —OR^{6a}, wherein
R^{6a} is —C₁₋₆alkyl optionally substituted with one R⁷, wherein
R⁷ is heterocyclyl, —OR^{7a}, or —NR^{7a}R^{7b}, wherein
R^{7a} and R^{7b} are each independently hydrogen, —C₁₋₆alkyl, C₁₋₆alkoxy-C₁₋₆alkyl-; and
Said heterocyclyl is optionally substituted with halogen, hydroxy, or —C₁₋₆alkyl.

36. The compound according to claim 35, wherein said heterocyclyl as R⁷ is a 4-, 5-, 6-, 7- or 8-membered heterocyclyl comprising one or two heteroatoms independently selected from nitrogen, oxygen or optionally oxidized sulfur as ring member(s), optionally substituted with —C₁₋₆ alkyl; preferably 5- or 6-membered heterocyclyl comprising one or two heteroatoms independently selected from nitrogen, oxygen or optionally oxidized sulfur as ring member(s).

37. The compound according to claim 36, wherein R⁷ is morpholinyl, morpholino, pyrrolidinyl, pyrrolidino, 4-methylpiperizinyl, or piperidinyl.

38. The compound according to claim 35, wherein R⁶ is —OR^{6a}, wherein R^{6a} is —C₁₋₆alkyl optionally substituted with one R⁷, wherein R⁷ is —OR^{7a}, wherein R^{7a} is hydrogen, —C₁₋₆alkyl, C₁₋₆alkoxy-C₁₋₆alkyl-.

39. The compound according to claim 35, wherein R⁶ is —OR^{6a}, wherein R^{6a} is —C₁₋₆alkyl optionally substituted with one R⁷, wherein R⁷ is —NR^{7a}R^{7b}, wherein R^{7a} and R^{7b} are hydrogen, or —C₁₋₆alkyl.

40. The compound according to claim 35, wherein R⁶ is methoxyethoxy-, methoxyethoxyethoxy-, 2-hydroxyethoxy, 2-hydroxypropoxy-, aminoethoxy-, N,N-dimethylaminoethoxy-, or N-methylaminoethoxy-.

41. The compound of claim 1, which is Compound Nos. A1, A2, A3, A4, A5, A6, A7, A8, A9, A10, A11, A12, A13, A14, A15, A16, A17, A18, A19, A20, A21, A22, A23, A24, A25, A26, A27, A28, A29, A30, A31, A32, A33, A34, A35, A36, A37, A38, A39, A40, A41, A42, A43, A44, A45, A46, A47, A48, A49, A50, A51, A52, A53, A54, A55, A56, A57, A58, A59, A60, A61, A62, A63, A64, A65, A66, A67, A68, A69, A70, A71, A72, A73, A74, A75, A76, A77, A78, A79, A80, A81, A82, A83, A84, A85, A86, A87, A88, A89, A90, A91, A92, A93, A94, A95, A96, A97, A98, A99, A100, A101, A102, A103, A104, A105, A106, A107, A108, B1, B2, B3, B4, B5, B6, B7, B8, B9, B10, B11, B12, B13, B14, B15, B16, B17, B18, B19, B20, B21, B22, B23, B24, B25, B26, B27, B28, B29, B30, B31, B32, B33, B34, C1, C2, C3, C4, C5, C6, C7, C8, C9, C10, C11, C12, C13, C14, C15, C16, C17, C18, C19, C20, C21, C22, C23, C24, C25, D1, or D2.

42. A pharmaceutical composition comprising the compound of any of claims 1-41 or a stereoisomer thereof, or a pharmaceutically acceptable salt thereof and a pharmaceutically acceptable excipient.

43. A method of treating cancer, comprising administering a subject in need thereof the compound of any of claims 1-41 or a stereoisomer thereof, or a pharmaceutically acceptable salt thereof.
